(19) United States
(12) Patent Application Publication
(10) Pub. No.: US 2026/0159498 A1
(43) Pub. Date: Jun. 11, 2026

(54) AKT3 MODULATORS

(71) Applicant: GEORGIAMUNE LLC, Gaithersburg, MD (US)

(72) Inventors: Samir Khleif, Bethesda, MD (US); Mikayel MKRTICHYAN, Tujunga, CA (US); Malcolm MACCOSS, Seabrook Island, SC (US)

(21) Appl. No.: 18/707,109

(22) PCT Filed: Nov. 4, 2022
(86) PCT No.: PCT/US2022/079334

§ 371 Date: May 2, 2024

Related U.S. Application Data
(60) Provisional application No. 63/276,218, filed on Nov. 5, 2021.

(51) Int. Cl. C07D 401/12; C07D 213/74; C07D 215/46; C07D 401/14; C07D 413/12; C07D 413/14; C07D 471/04

(52) U.S. Cl. CPC C07D 401/12; C07D 213/74; C07D 215/46; C07D 401/14; C07D 413/12; C07D 413/14; C07D 471/04

(57) ABSTRACT

Compounds of Formula (Ia), (Ib), or (Ic), are described, where the various substituents are defined herein. The compounds can modulate a property or effect of Akt3 in vitro or in vivo, and can also be used, individually or in combination with other agents, in the prevention or treatment of a variety of conditions. Methods for synthesizing the compounds are described. Pharmaceutical compositions and methods of using these compounds or compositions, individually or in combination with other agents or compositions, in the prevention or treatment of a variety of conditions are also described.

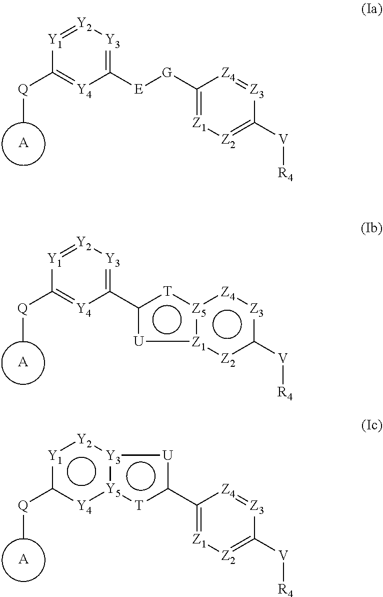

### CROSS-REFERENCE TO RELATED APPLICATIONS

This application claims the benefit and priority of U.S. Provisional Application No. 63/276,218, filed on Nov. 5, 2021, the content of which is incorporated herein by reference in its entirety.

### INCORPORATION BY REFERENCE

Any patent, patent publication, journal publication, or other document cited herein is expressly incorporated herein by reference in its entirety.

### FIELD OF THE INVENTION

This invention is generally related to Akt3 modulators and methods for treating and preventing diseases by modulating Akt3 signaling.

### BACKGROUND OF THE INVENTION

Chronic illnesses and diseases are long-lasting conditions that require ongoing medical attention and typically negatively affect the patient's quality of life. Chronic diseases are a leading cause of disability and death in the U.S. Common chronic diseases include, but are not limited to, heart disease, cancer, neurodegenerative diseases, diabetes, obesity, eating disorders, and arthritis. It is estimated that roughly 6 in 10 adults in the U.S. have a chronic disease, with 4 in 10 having two or more chronic diseases. Chronic diseases are also a leading driver of the U.S.'s $3.3 trillion annual health care costs (see “About Chronic Diseases”, National Center for Chronic Disease Prevention and Health Promotion, Centers for Disease Control and Prevention; updated Oct. 23, 2019). These statistics emphasize the need for new and improved treatments and prophylactic interventions for diseases such as, for example, cancer, inflammatory disease, neurodegenerative disease, pathogenic infection, immunodeficiency disorder, weight gain disorder, weight loss disorder, hormone imbalance, tuberous sclerosis, retinitis pigmentosa, and congestive heart failure.

Neurodegenerative diseases are debilitating conditions that are characterized by the progressive degeneration and death of nerve cells, also called neurons. Neurons are the building blocks of the nervous system and do not usually self-replenish following damage or death. The loss or dysfunction of neurons in patients with neurodegenerative disease can affect body movement and brain function. Neurodegenerative diseases include, but are not limited, to Alzheimer's disease, amyotrophic lateral sclerosis, Huntington's disease, Parkinson's disease, multiple sclerosis, prion disease, motor neuron disease, spinocerebellar ataxia, and spinal muscular atrophy. The symptoms of advanced neurodegenerative diseases can be devastating, with patients losing memory, control over movements, and personality. Existing treatments for neurodegenerative diseases can manage symptoms but generally cannot prevent or cure the disease. Such existing treatments typically have negative side effects which lead to further deterioration of patient quality of life.

A serious complication of chronic diseases such as neurodegenerative diseases and cancer is cachexia, also called wasting syndrome. Cachexia is defined as weight loss greater than 5% of body weight in 12 months or less in the presence of chronic illness. Other symptoms of cachexia include muscle atrophy, fatigue, weakness, and, often, loss of appetite. The weight loss associated with cachexia is due to the loss of not only fat but also muscle mass. Patients with cachexia often lose weight even if they are still eating a normal diet. Like neurodegenerative diseases, there are currently no effective treatments for cachexia, which contributes to a large number of chronic disease-related deaths.

Thus, there is an unmet need for more effective and tolerable treatments and prophylactic interventions for these and other diseases and complications associated with the diseases.

### SUMMARY OF THE INVENTION

As used herein, Akt3 is RAC-gamma serine/threonine-protein kinase, which is an enzyme that, in humans, is encoded by the Akt3 gene. In one aspect, a compound having a structure of Formula Ia, Ib, or Ic

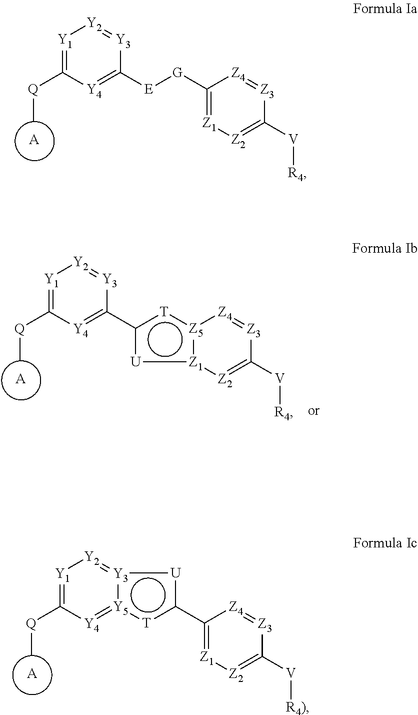

or a salt thereof, is described, where the various substituents are defined herein. In certain embodiments, the compound can modulate a property or effect of Akt3 in vitro or in vivo, and/or can also be used, individually or in combination with other agents, in the prevention or treatment of a variety of conditions. In other embodiments, methods for synthesizing the compounds are provided. In another aspect, pharmaceutical compositions including the compound and methods of using these compositions, individually or in combination with other agents or compositions, in the prevention or treatment of a variety of conditions are also described herein.

In one aspect, a compound of Formula Ia, Ib, or Ic,

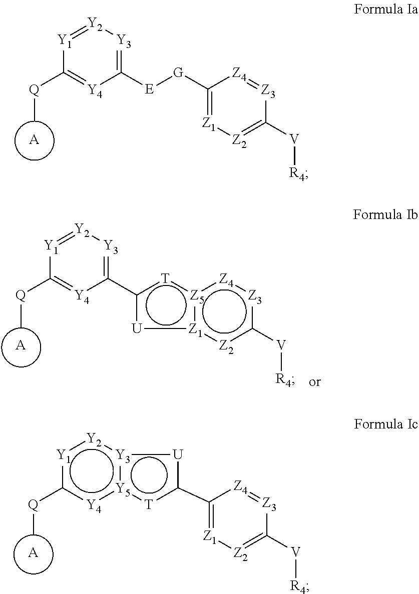

or a pharmaceutically acceptable salt thereof is described,
wherein:
\textcircled{A} is

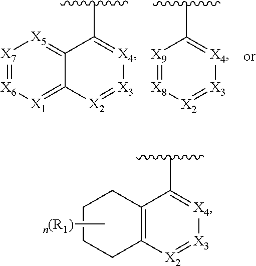

each occurrence of X₁, X₂, X₃, X₄, X₅, X₆, X₇, X₈, and X₉ is independently CR₁ or N;
each occurrence of R₁ is independently selected from the group consisting of H, D, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₇)cycloalkyl, (C₄-C₁₀)bicycloalkyl, (C₄-C₁₄)tricycloalkyl, (C₃-C₇)heterocycloalkyl, halogenated (C₃-C₇)heterocycloalkyl, (C₄-C₁₀)heterobicycloalkyl, (C₄-C₁₄)heterotricycloalkyl, (C₄-C₁₀)heterospiroalkyl, (C₃-C₇)cycloalkenyl, (C₃-C₇)heterocycloalkenyl, (C₄-C₁₀)bicycloalkenyl, (C₄-C₁₀)heterobicycloalkenyl, (C₄-C₁₄)tricycloalkenyl, (C₄-C₁₄)heterotricycloalkenyl, aryl, heteroaryl, —ORₐ, —SRₐ, —N(Rₐ)₂, —CORₐ, —CO₂Rₐ, CON(Rₐ)₂, —CN, —NC, NO₂, N₃, —SO₂Rₐ, —SO₂N(Rₐ)₂, —N(Rₐ)SO₂Rₐ,

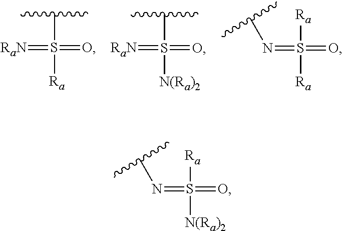

and a partially saturated bicyclic heteroaryl optionally substituted by one or more (C₁-C₆)alkyl, halogenated (C₁-C₆)alkyl, —SO₂Rₐ, or —SO₂N(Rₐ)₂;
wherein the (C₃-C₇)cycloalkyl, (C₄-C₁₀)bicycloalkyl, (C₄-C₁₄)tricycloalkyl, (C₃-C₇)heterocycloalkyl, halogenated (C₃-C₇)heterocycloalkyl, (C₄-C₁₀)heterobicycloalkyl, (C₄-C₁₄)heterotricycloalkyl, (C₄-C₁₀)heterospiroalkyl, (C₃-C₇)cycloalkenyl, (C₃-C₇)heterocycloalkenyl, (C₄-C₁₀)bicycloalkenyl, (C₄-C₁₀)heterobicycloalkenyl, (C₄-C₁₄)tricycloalkenyl, (C₄-C₁₄)heterotricycloalkenyl, aryl, and heteroaryl of R₁ are each optionally substituted by one or more (C₁-C₆)alkyl, halogenated (C₁-C₆)alkyl, halogen, —ORₐ, —CN, or —N(Rₐ)₂;
n is an integer from 0-4 where valence permits;
Q is C(Rₐ)₂, O, NRₐ, N(C═O)Rₐ, or NSO₂Rₐ;
Y₁, Y₂, Y₃, Y₄ and Y₅ are each independently N or CR₂ where valance permits;
R₂ is selected from the group consisting of H, halogen, D, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₇)cycloalkyl, (C₄-C₁₀)bicycloalkyl, (C₃-C₇)heterocycloalkyl, halogenated (C₃-C₇)heterocycloalkyl, (C₄-C₁₀)heterobicycloalkyl, (C₄-C₁₀)heterospiroalkyl, aryl, heteroaryl, —ORₐ, —SRₐ, —N(Rₐ)₂, —CORₐ, —CO₂Rₐ, CON(Rₐ)₂, —CN, —NC, NO₂, N₃, —SO₂Rₐ, —SO₂N(Rₐ)₂, —N(Rₐ)SO₂Rₐ,

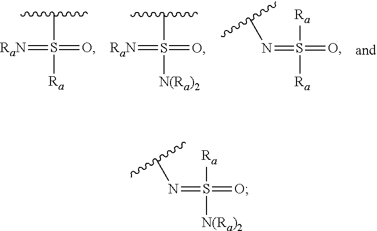

-E-G- is —(C═O)NRₓ—, —NRₓ(C═O)—, —N(Rₓ)(C═O)N(Rₓ)—, —O(C═O)N(Rₓ)—, —N(Rₓ)(C═O)O—, —SO₂NRₓ—, —NRₓSO₂—, or

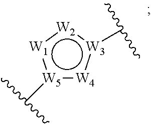

wherein;
each occurrence of Rₓ is independently H, (C₁-C₆)alkyl, (C₃-C₇)cycloalkyl, aryl, or heteroaryl; or wherein Rₓ and Y₃, Rₓ and Y₄, Rₓ and Z₁, or Rₓ and Z₄ taken together form a 5-6-membered heterocycle optionally substituted by one or more R_{z}, wherein each occurrence of R_{z} is independently H, (C₁-C₆)alkyl, OH, O(C₁-C₆)alkyl, or halogen; or two R_{z} taken together form a 4-6-membered ring optionally substituted with halogen or (C₁-C₆)alkyl;
W₁, W₂, W₃, W₄, and W₅ are each independently CR₆, N, or NR₆ where valence permits;
each occurrence of R₆ is independently selected from the group consisting of H, halogen, (C₁-C₆)alkyl, and (C₁-C₆)haloalkyl;
each occurrence of T is independently O, N, NRₐ, N(C═O)Rₐ, NC(R_{b})₂OP(═O)(OR_{b})₂, or NSO₂Rₐ where valance permits;
each occurrence of U is independently O, N, NRₐ, N(C═O)Rₐ, NC(R_{b})₂OP(═O)(OR_{b})₂, or NSO₂Rₐ where valance permits;
each occurrence of R_{b} is independently H or (C₁-C₆)alkyl;
Z₁, Z₂, Z₃, Z₄ and Z₅ are each independently N or CR₃ where valance permits;
R₃ is selected from the group consisting of H, D, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₇)cycloalkyl, (C₄-C₁₀)bicycloalkyl, (C₃-C₇)heterocycloalkyl, halogenated (C₃-C₇)heterocycloalkyl, (C₄-C₁₀)heterobicycloalkyl, (C₄-C₁₀)heterospiroalkyl, aryl, heteroaryl, —ORₐ, —SRₐ, —N(Rₐ)₂, —CORₐ, —CO₂Rₐ, CON(Rₐ)₂, —CN, —NC, NO₂, N₃, —SO₂Rₐ, —SO₂N(Rₐ)₂, —N(Rₐ)SO₂Rₐ,

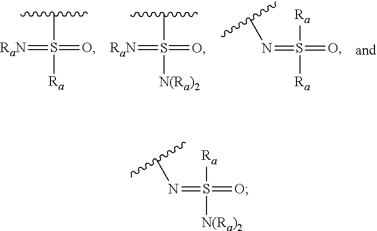

V is absent, C(Rₐ)₂, NRₐ, N(C═O)Rₐ, NSO₂Rₐ or O;
R₄ is selected from the group consisting of (C₁-C₆)alkyl, (C₃-C₇)cycloalkyl, (C₄-C₁₀)bicycloalkyl, (C₃-C₇)heterocycloalkyl, (C₄-C₁₀)heterobicycloalkyl, (C₄-C₁₀)heterospiroalkyl, aryl, and heteroaryl, each optionally substituted with one or more R₅;
or alternatively V and R₄ taken together form a (C₃-C₇)heterocycloalkyl or (C₄-C₁₀)heterospiroalkyl;
each occurrence of R₅ is independently selected from the group consisting of H, D, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₇)cycloalkyl, (C₄-C₁₀)bicycloalkyl, (C₃-C₇)heterocycloalkyl, halogenated (C₃-C₇)heterocycloalkyl, (C₄-C₁₀)heterobicycloalkyl, (C₄-C₁₀)heterospiroalkyl, aryl, heteroaryl, —ORₐ, —SRₐ, —N(Rₐ)₂, —CORₐ, —CO₂Rₐ, CON(Rₐ)₂, —CN, —NC, NO₂, N₃, —SO₂Rₐ, —SO₂N(Rₐ)₂, —N(Rₐ)SO₂Rₐ, N(Rₐ)CORₐ,

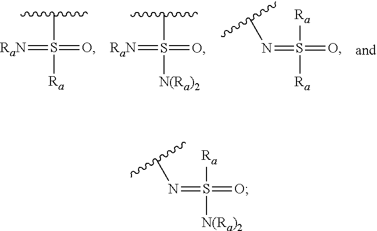

and
each occurrence of Rₐ is independently H, (C₁-C₆)alkyl, (C₂-C₆)alkenyl, (C₃-C₇)cycloalkyl, aryl, or heteroaryl, or two Rₐ taken together form a 4-6-membered ring optionally substituted with halogen or (C₁-C₆)alkyl.

In any one of the embodiments disclosed herein, Q, T, and U are each independently O, NH, NCH₃, N(C═O)H, N(C═O)CH₃, N(C═O)CH₂CH₃, NSO₂CH₃, or NSO₂CH₂CH₃, where valence permits. In any one of the embodiments disclosed herein, T, and U are each independently O, N, NH, NCH₃, N(C═O)H, N(C═O)CH₃, N(C═O)CH₂CH₃, NSO₂CH₃, or NSO₂CH₂CH₃, where valence permits.

In any one of the embodiments disclosed herein, X₁, X₂, X₃, X₄, X₅, X₆, X₇, X₈, X₉, Y₁, Y₂, Y₃, Y₄, Y₅, Z₁, Z₂, Z₃, Z₄, and Z₅ are each independently CH, C (halogen), or N.

In any one of the embodiments disclosed herein, \textcircled{A} is

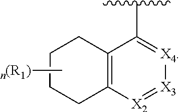

In any one of the embodiments disclosed herein, the structural moiety

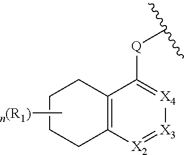

has the structure of

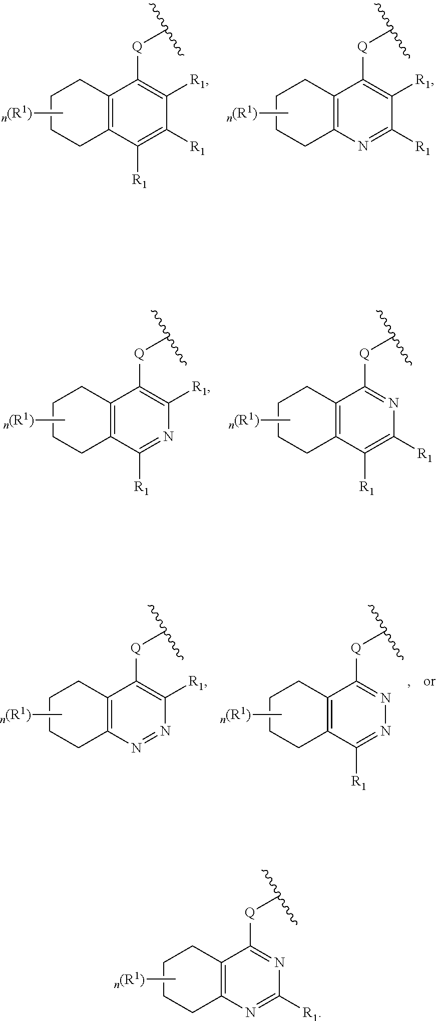

In any one of the embodiments disclosed herein, n is 0, 1, or 2.

In any one of the embodiments disclosed herein, the structural moiety

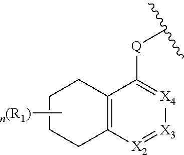

has the structure of

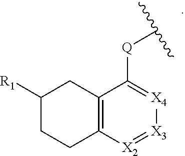

In any one of the embodiments disclosed herein, the structural moiety

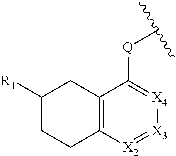

has the structure of

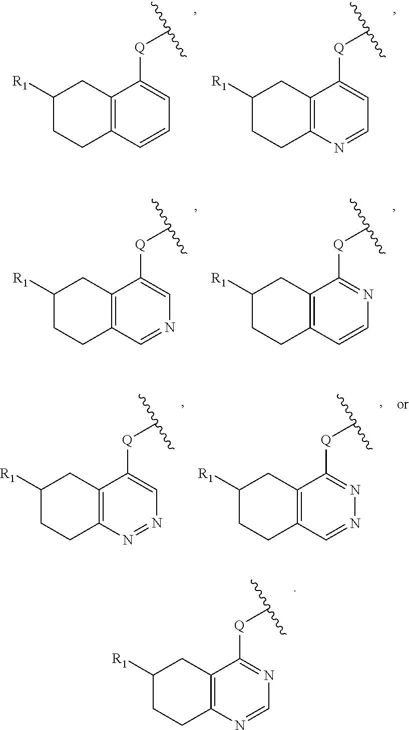

In any one of the embodiments disclosed herein, \textcircled{A} is

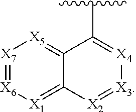

In any one of the embodiments disclosed herein, the structural moiety

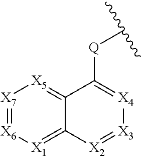

has the structure of

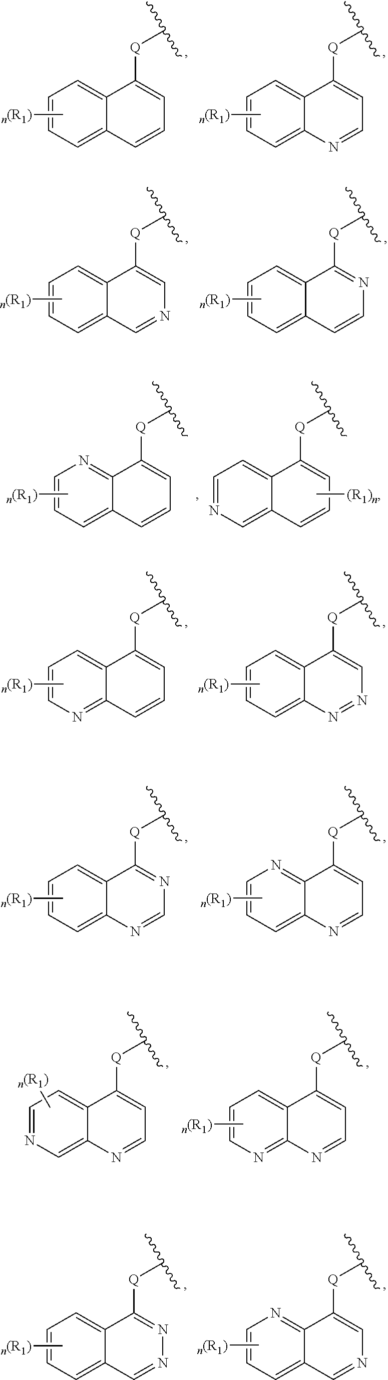

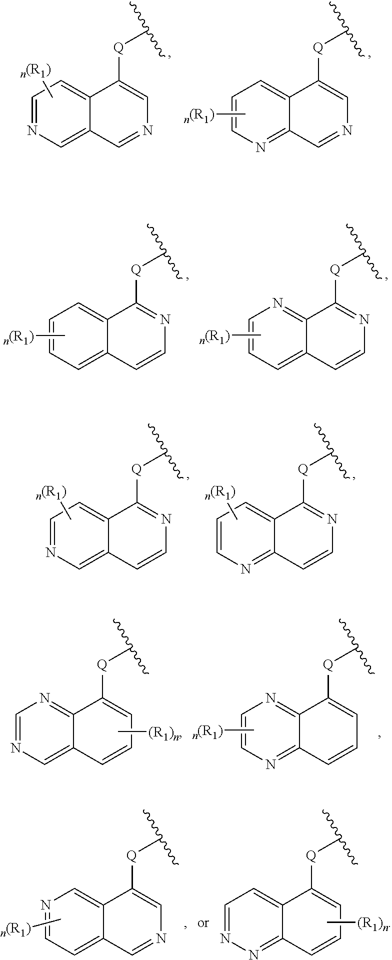

In any one of the embodiments disclosed herein, n is 0, 1, or 2.

In any one of the embodiments disclosed herein, the structural moiety

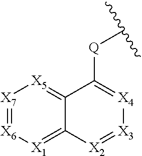

has the structure of

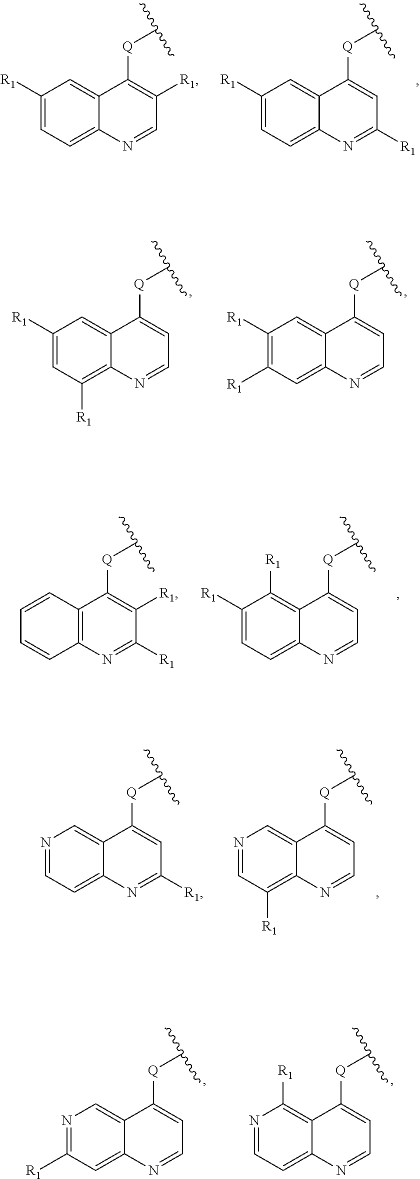

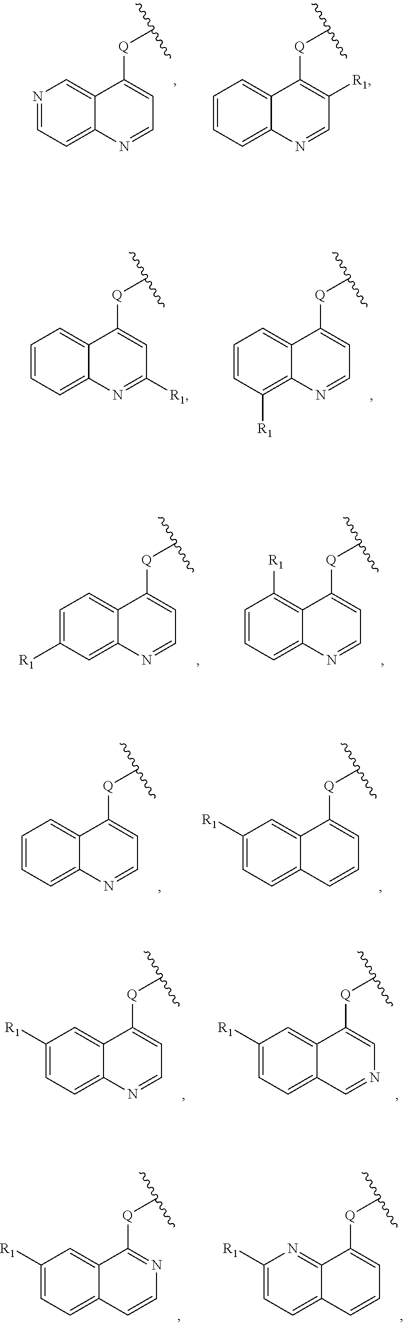

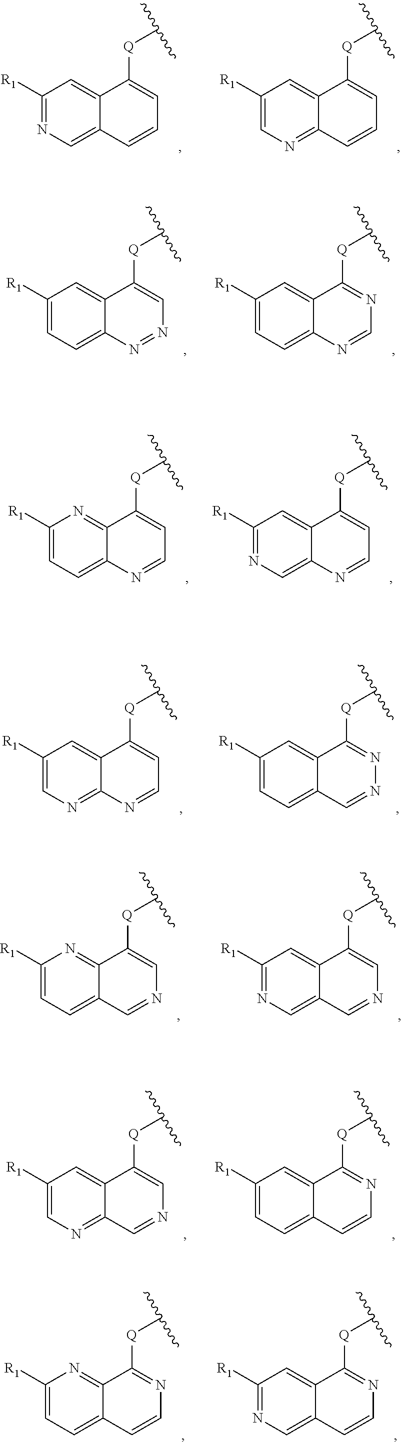

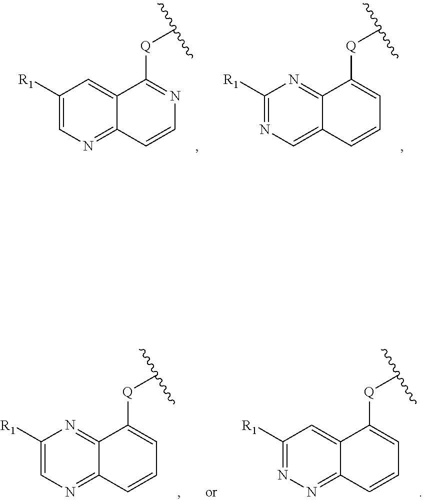

In any one of the embodiments disclosed herein, the structural moiety

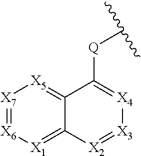

has the structure of

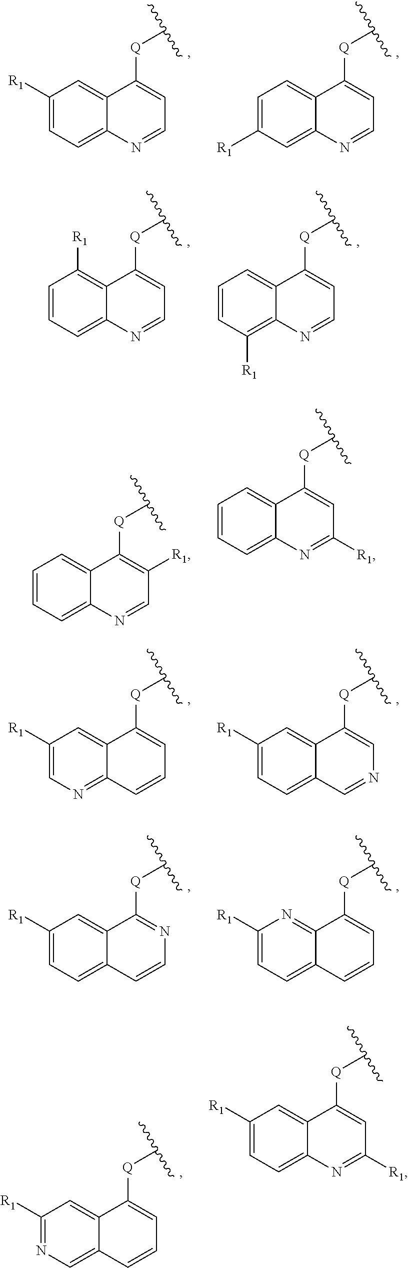

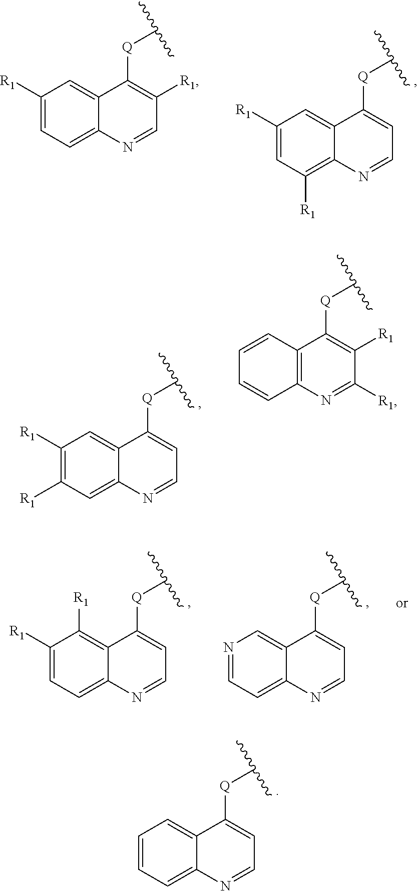

In any one of the embodiments disclosed herein, the structural moiety

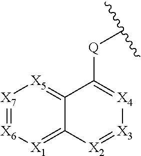

has the structure of

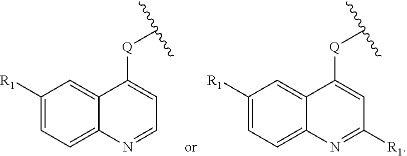

In any one of the embodiments disclosed herein, \textcircled{A} is

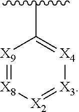

In any one of the embodiments disclosed herein, the structural moiety

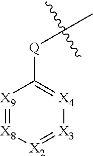

has the structure of

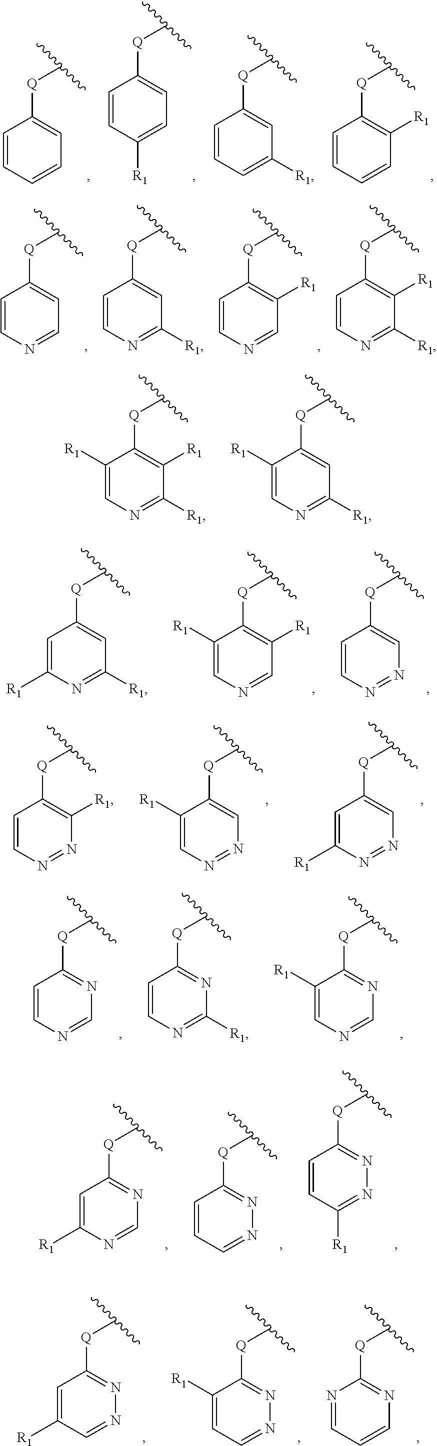

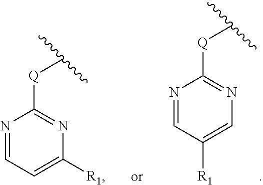

In any one of the embodiments disclosed herein, the structural moiety

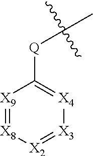

has the structure of

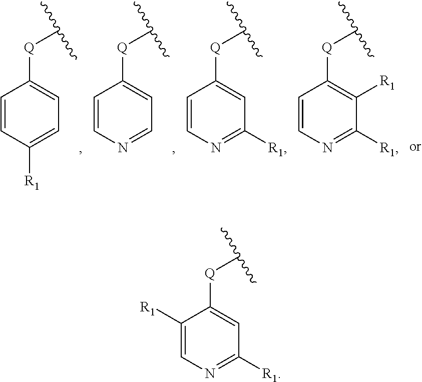

In any one of the embodiments disclosed herein, Q is O.

In any one of the embodiments disclosed herein, Q is NRₐ, N(C═O)Rₐ, or NSO₂Rₐ.

In any one of the embodiments disclosed herein, each occurrence of R₁ is independently H, D, halogen, ORₐ, N(Rₐ)₂, (C₁-C₆)alkyl, (C₁-C₆)alkynyl, (C₃-C₇)heterocycloalkyl, (C₄-C₁₀)heterospiroalkyl, halogenated (C₃-C₇)heterocycloalkyl, aryl, (C₄-C₁₀)bicycloalkyl, —CN, —NC, N₃, NO₂, CORₐ, CO₂Rₐ, CON(Rₐ)₂, —SO₂Rₐ, or —SO₂N(Rₐ)₂; wherein the (C₃-C₇)heterocycloalkyl is optionally substituted with one or more (C₁-C₆)alkyl.

In any one of the embodiments disclosed herein, each occurrence of R₁ is independently H, halogen, (C₁-C₆)alkyl, (C₃-C₇)heterocycloalkyl, (C₄-C₁₀)heterospiroalkyl, halogenated (C₃-C₇)heterocycloalkyl, N(Rₐ)₂, or —CN; wherein the (C₃-C₇)heterocycloalkyl is optionally substituted with one or more (C₁-C₆)alkyl.

In any one of the embodiments disclosed herein, each occurrence of R₁ is independently H, halogen, (C₁-C₆)alkyl, (C₁-C₆)alkyl, halogenated (C₃-C₇)heterocycloalkyl, or (C₃-C₇)heterocycloalkyl; wherein the (C₃-C₇)heterocycloalkyl is optionally substituted with one or more (C₁-C₆)alkyl.

In any one of the embodiments disclosed herein, each occurrence of R₁ is independently H, D, F, Cl, Br, CH₃, OCH₃, NH₂, NHCH₃, N(CH₃)₂,

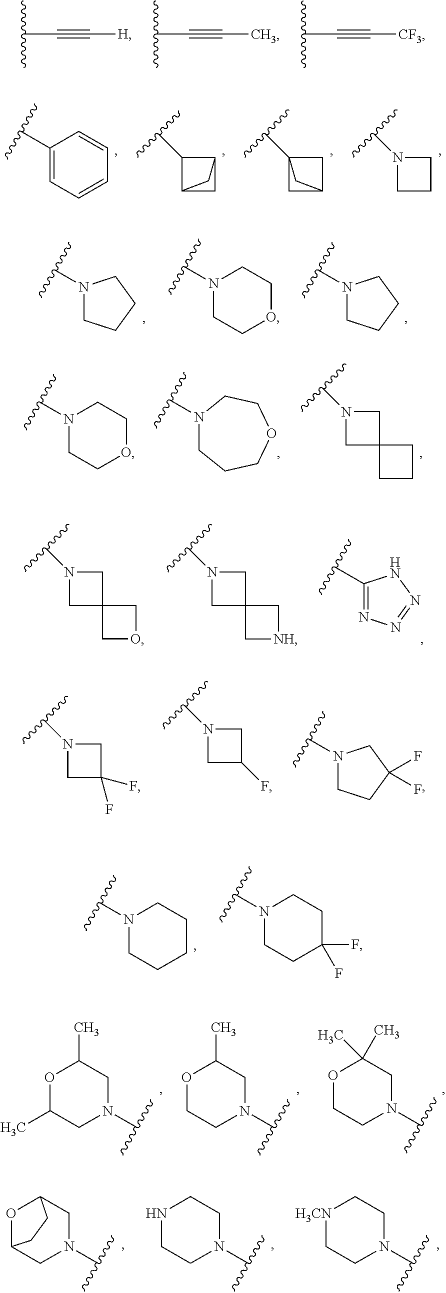

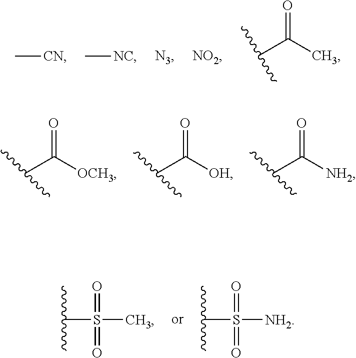

In any one of the embodiments disclosed herein, each occurrence of R₁ is independently H, D, F, CH₃, NH₂, NHCH₃, N(CH₃)₂,

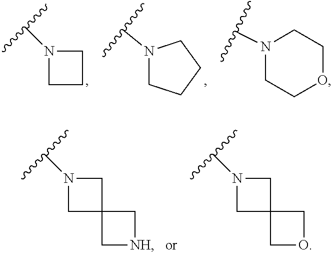

In any one of the embodiments disclosed herein, at least one occurrence of R₁ is

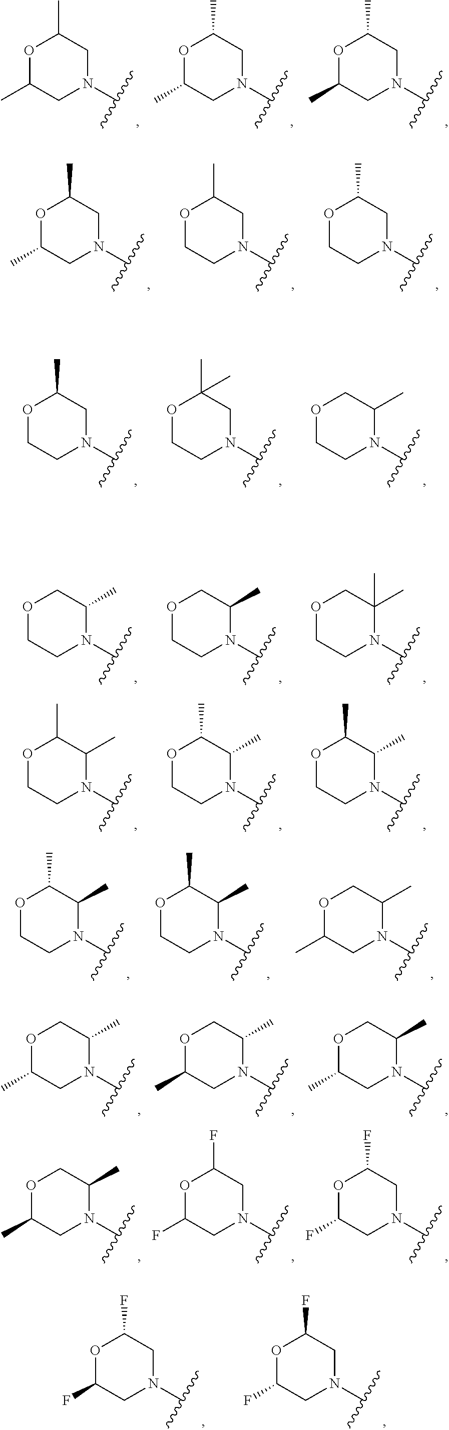

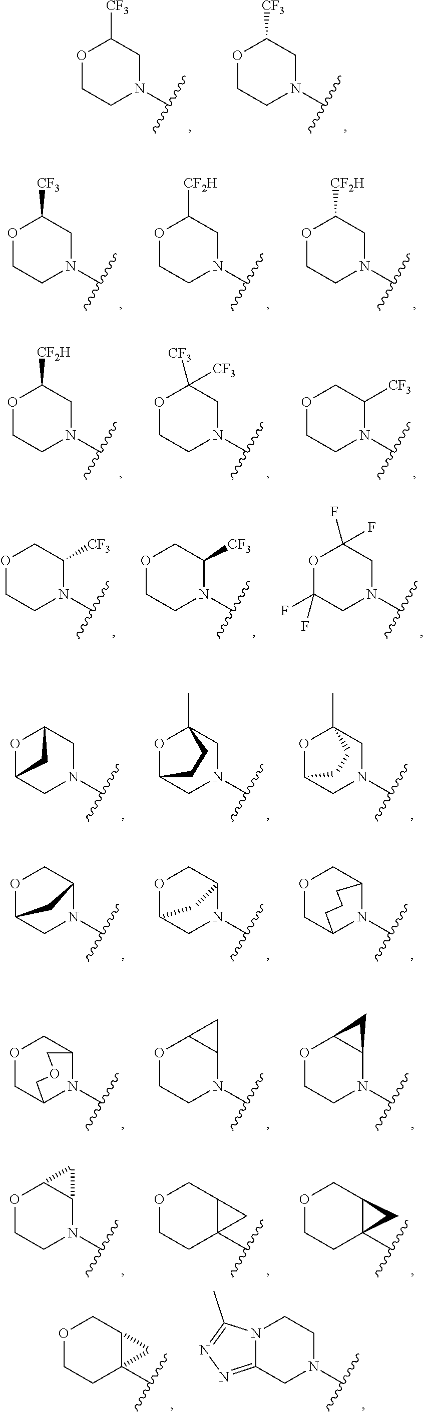

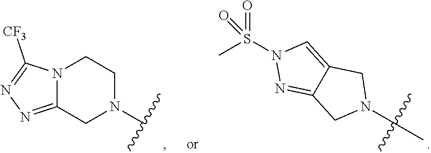 [SMILES: *N1CCn2c(nnc2C(F)(F)F)C1 ; *N1Cc2cn(S(C)(=O)=O)nc2C1]

In any one of the embodiments disclosed herein, at least one occurrence of R₁ is

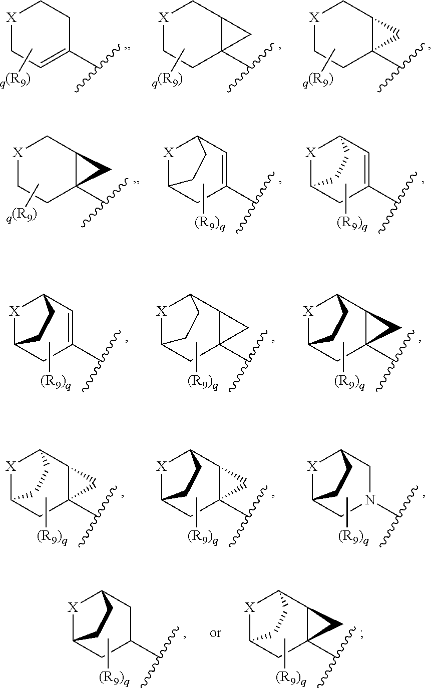

wherein X is CR₁₅, O, NR₁₄, or S; each occurrence of R₉ is independently H, (C₁-C₆)alkyl, halogenated (C₁-C₆)alkyl, halogen, —ORₐ, —CN, or —N(Rₐ)₂; R₁₄ is H, (C₁-C₆)alkyl, (C₃-C₇)cycloalkyl, (C₃-C₇)heterocycloalkyl, aryl, or heteroaryl; each occurrence of R₁₅ is independently H, (C₁-C₆)alkyl, halogenated (C₁-C₆)alkyl, halogen, —ORₐ, —CN, or —N(Rₐ)₂; and q is 0, 1, 2, or 3.

In any one of the embodiments disclosed herein, X is O.

In any one of the embodiments disclosed herein, each occurrence of R₉ is independently H, (C₁-C₆)alkyl, halogenated (C₁-C₆)alkyl, or halogen.

In any one of the embodiments disclosed herein, each occurrence of R₉ is independently H, F, Cl, Br, CH₃, CF₃, OH, NH₂, —NHCH₃, or —N(CH₃)₂.

In any one of the embodiments disclosed herein, each occurrence of R₉ is independently H, F, Cl, Br or CH₃.

In any one of the embodiments disclosed herein, each occurrence of R₁₅ is independently H, (C₁-C₆)alkyl, halogenated (C₁-C₆)alkyl, or halogen.

In any one of the embodiments disclosed herein, each occurrence of R₁₅ is independently H, F, Cl, Br or CH₃.

In any one of the embodiments disclosed herein, q is 0.

In any one of the embodiments disclosed herein, q is 1.

In any one of the embodiments disclosed herein, q is 2 or 3.

In any one of the embodiments disclosed herein, X is NR₁₄ and R₁₄ is H or (C₁-C₆)alkyl.

In any one of the embodiments disclosed herein, at least one occurrence of R₁ is

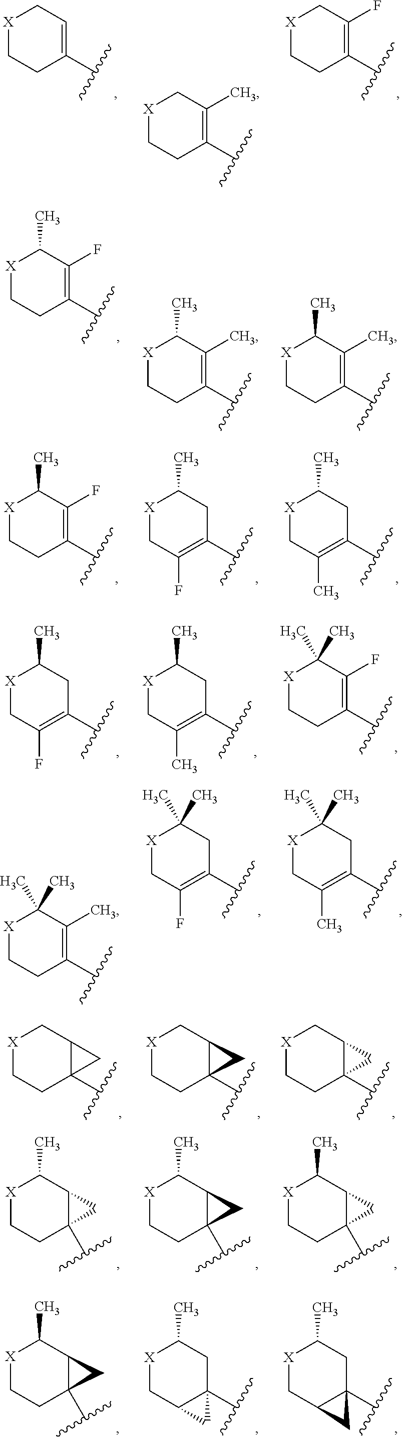

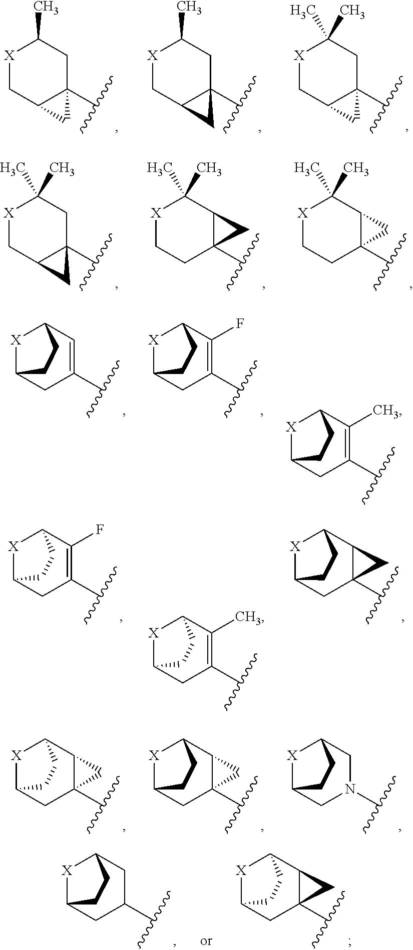

wherein X is O or NR₁₄; and R₁₄ is H or (C₁-C₆)alkyl.

In any one of the embodiments disclosed herein, at least one occurrence of R₁ is

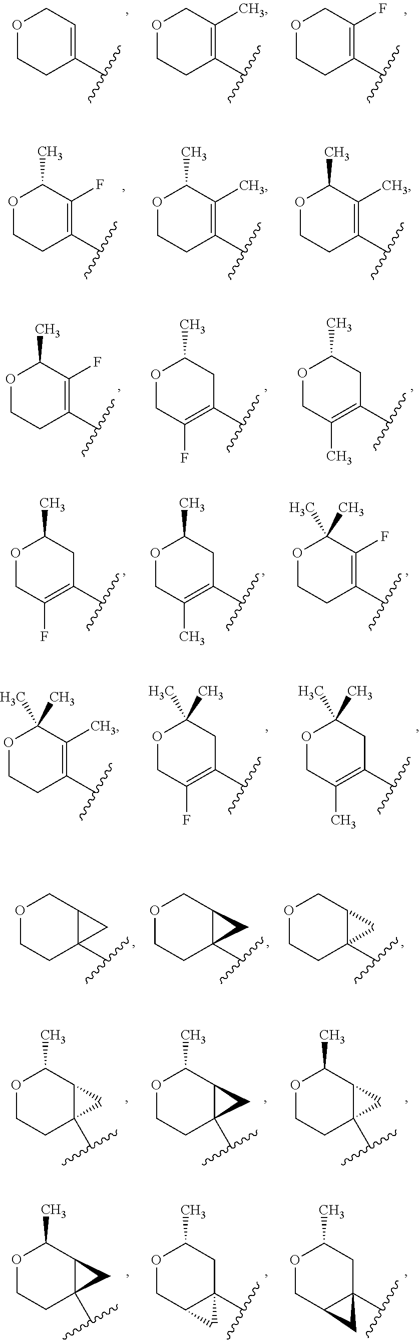

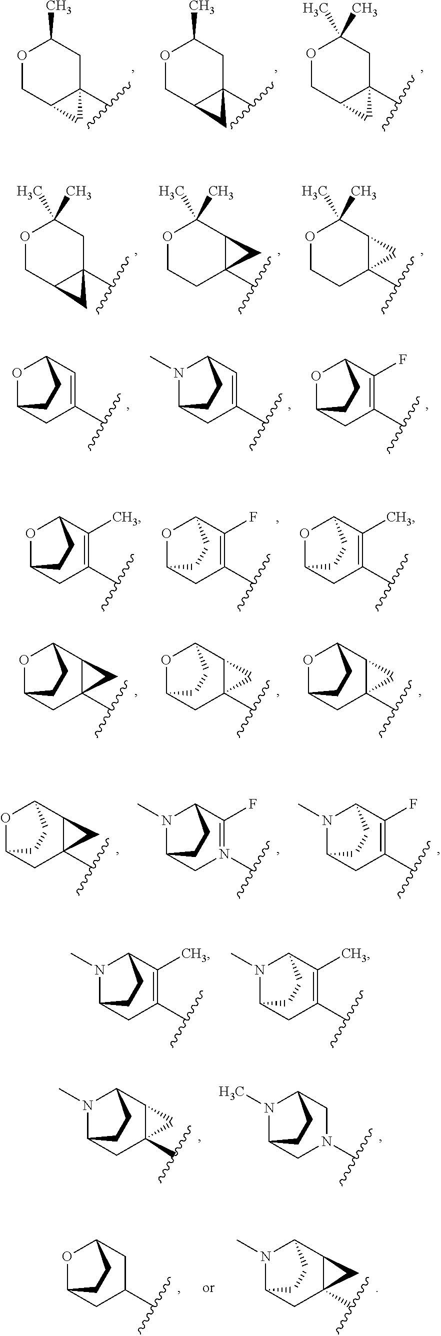

In any one of the embodiments disclosed herein, \textcircled{A} is

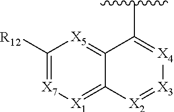

wherein R₁₂ is (C₃-C₇)cycloalkenyl, (C₃-C₇)heterocycloalkenyl, (C₄-C₁₀)bicycloalkenyl, (C₄-C₁₀)heterobicycloalkenyl, (C₄-C₁₄)tricycloalkenyl, or (C₄-C₁₄)heterotricycloalkenyl, each of which is optionally substituted by one or more (C₁-C₆)alkyl, halogenated (C₁-C₆)alkyl, halogen, —ORₐ, —CN, or —N(Rₐ)₂.

In any one of the embodiments disclosed herein, R₁₂ is

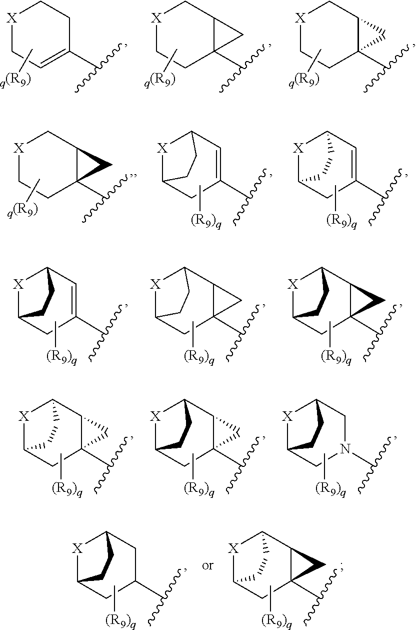

wherein X is CR₁₅, O, NR₁₄, or S; each occurrence of R₉ is independently H, (C₁-C₆)alkyl, halogenated (C₁-C₆)alkyl, halogen, —ORₐ, —CN, or —N(Rₐ)₂; R₁₄ is H, (C₁-C₆)alkyl, (C₃-C₇)cycloalkyl, (C₃-C₇)heterocycloalkyl, aryl, or heteroaryl; each occurrence of R₁₅ is independently H, (C₁-C₆)alkyl, halogenated (C₁-C₆)alkyl, halogen, —ORₐ, —CN, or —N(Rₐ)₂; and q is 0, 1, 2, or 3.

In any one of the embodiments disclosed herein, R₁₂ is

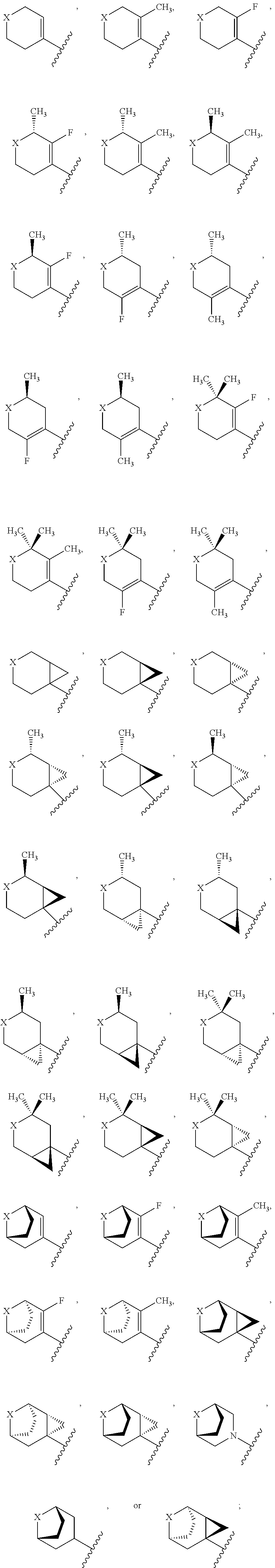

wherein X is O or NR₁₄; and R₁₄ is H or (C₁-C₆)alkyl.

In any one of the embodiments disclosed herein, R₁₂ is

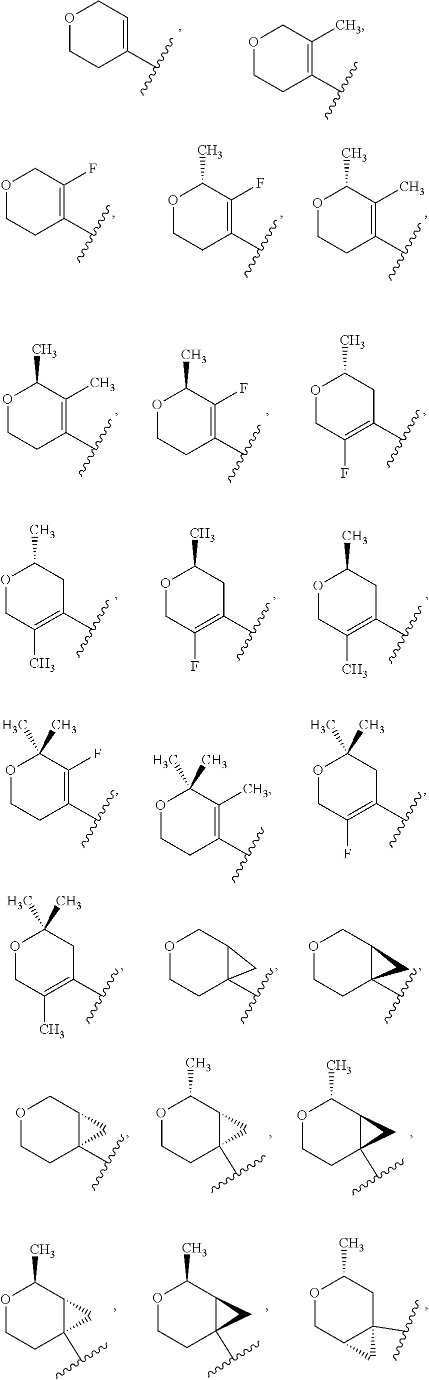

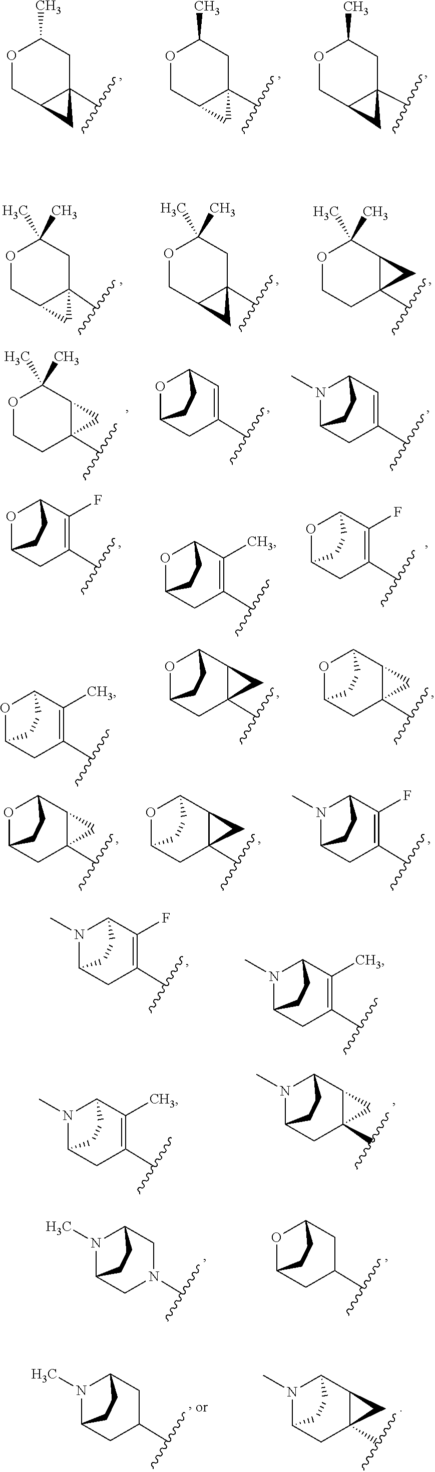

In any one of the embodiments disclosed herein, \textcircled{A} is

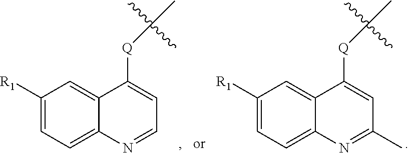

In any one of the embodiments disclosed herein, the structural moiety

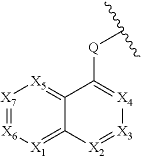

has the structure of

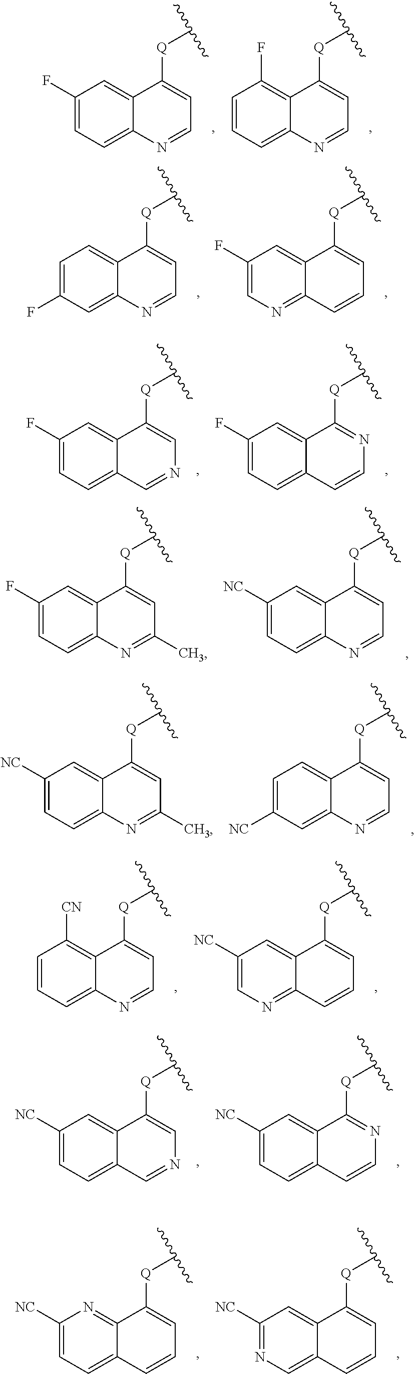

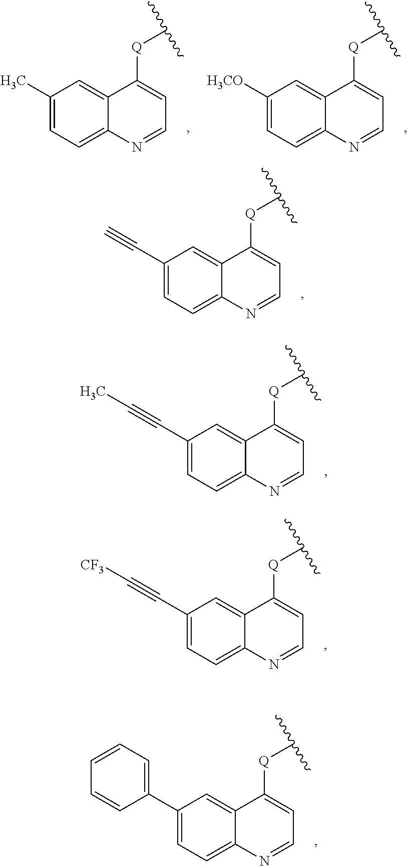

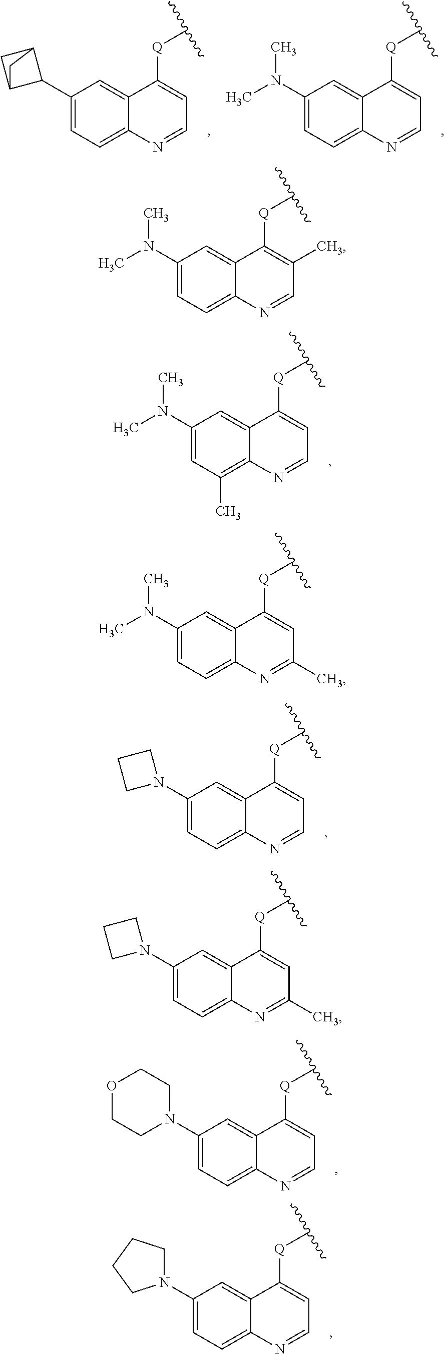

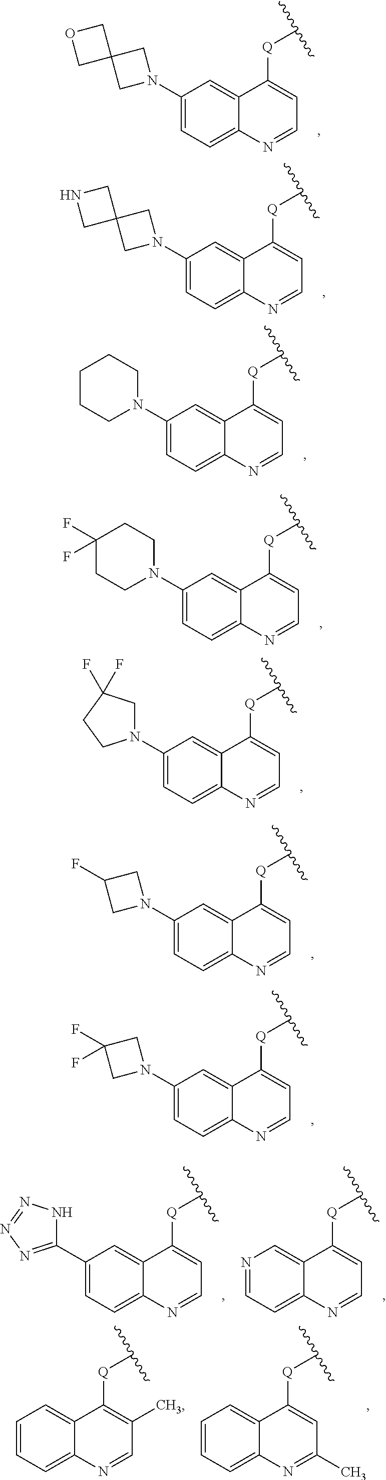

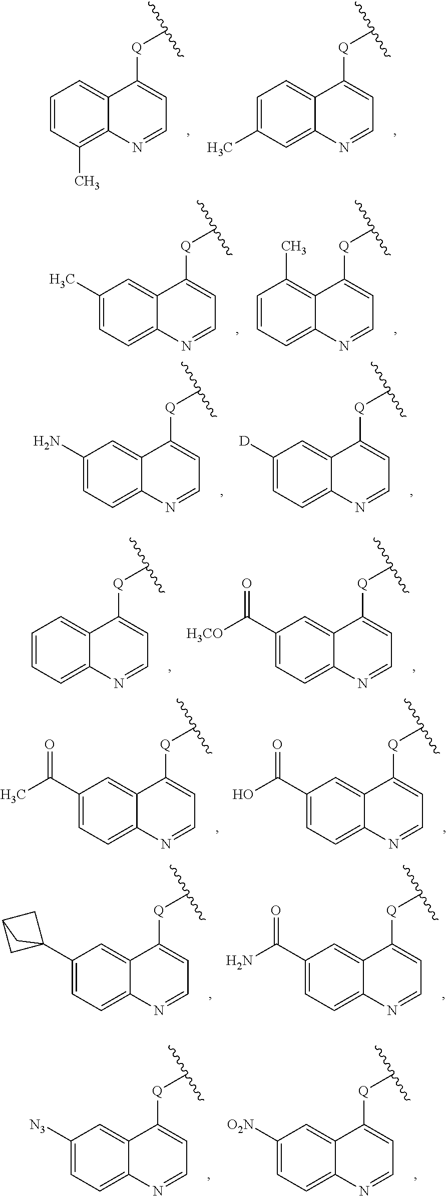

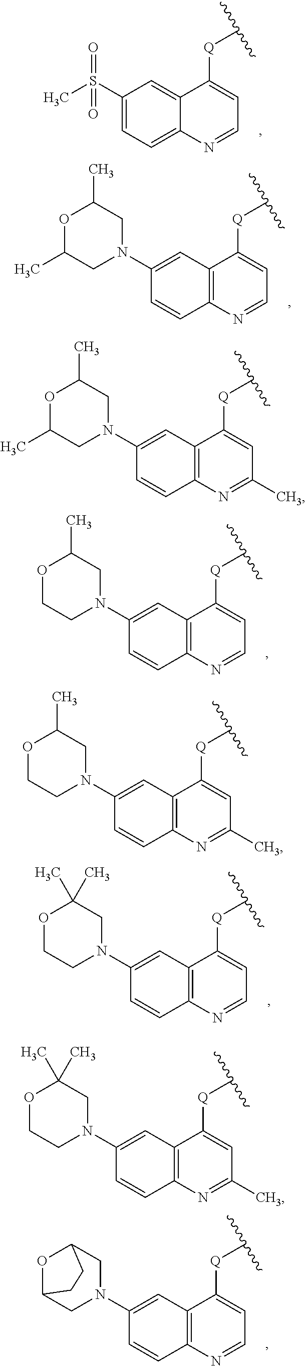

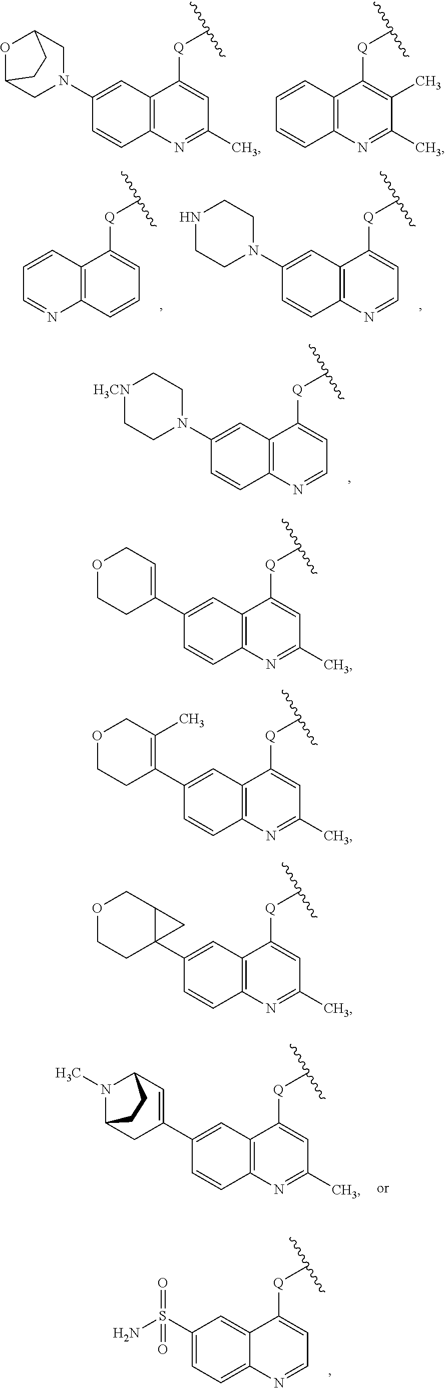

wherein Q is O or NH.

In any one of the embodiments disclosed herein, the structural moiety

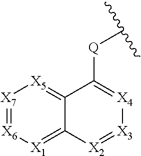

has the structure of

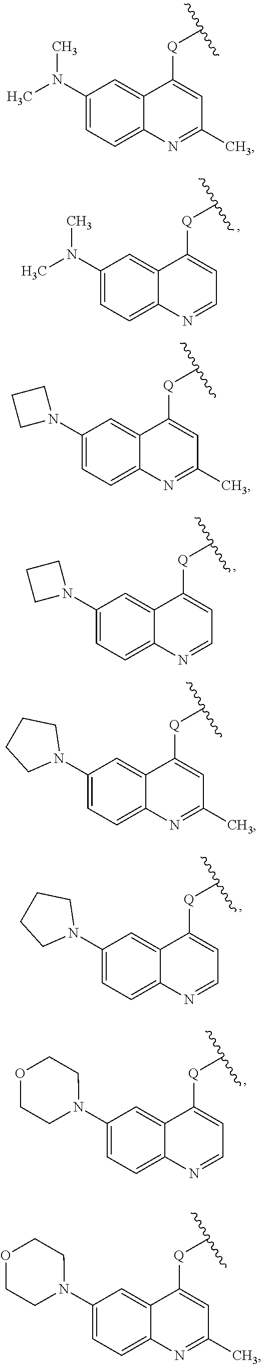

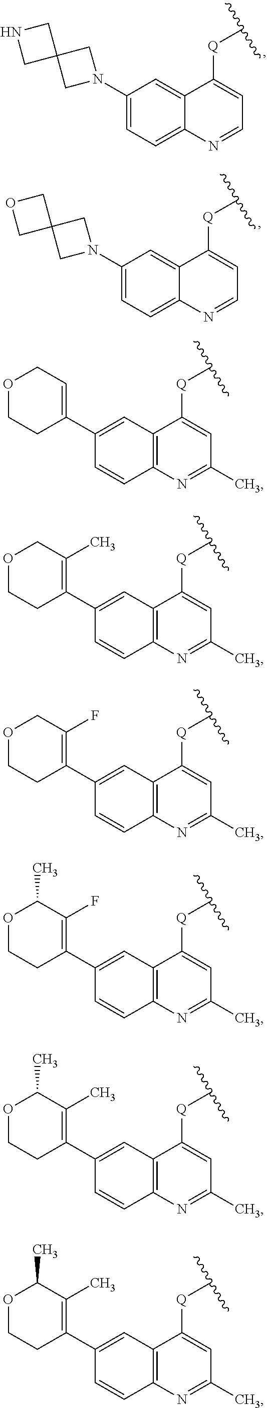

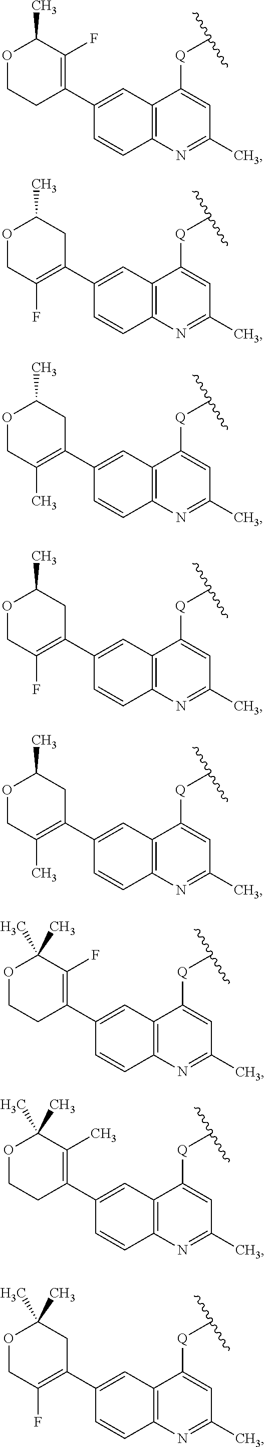

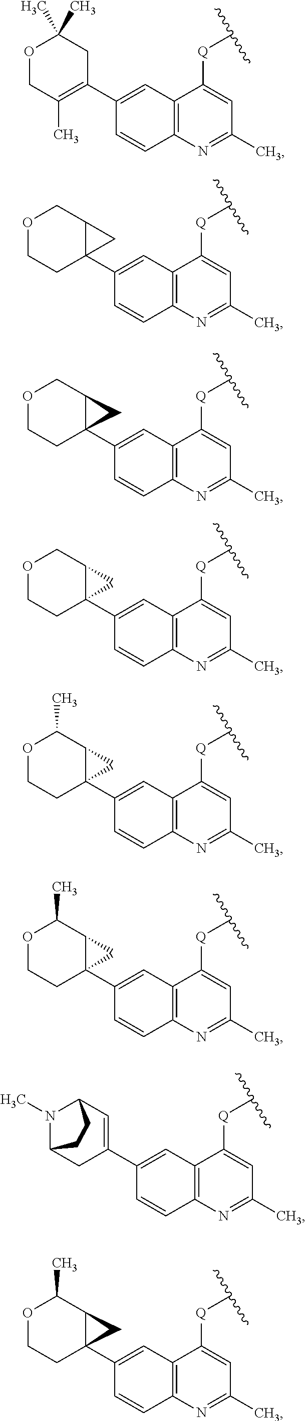

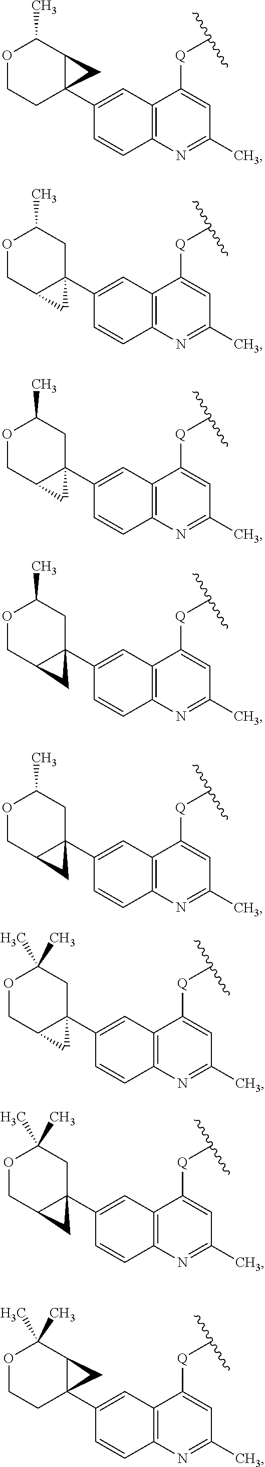

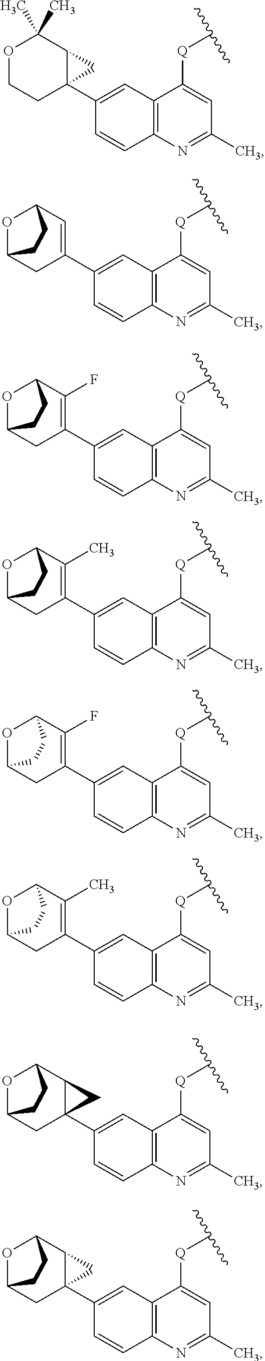

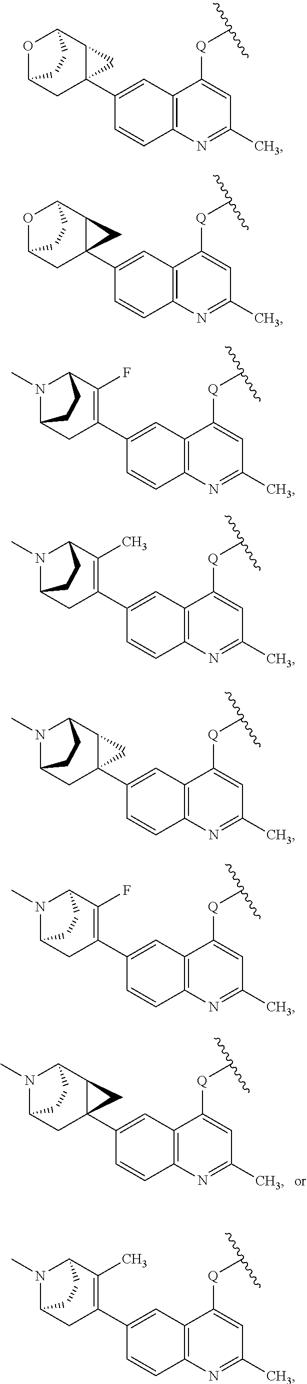

wherein Q is O or NH.

In any one of the embodiments disclosed herein, the structural moiety

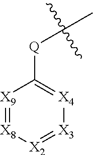

has the structure of

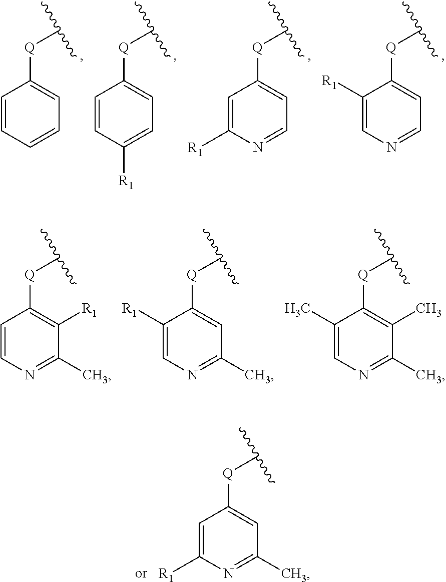

wherein Q is O or NH and R₁ is H, (C₁-C₆)alkyl, (C₃-C₇)heterocycloalkyl, halogenated (C₃-C₇)heterocycloalkyl, or halogen.

In any one of the embodiments disclosed herein, the structural moiety

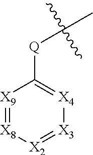

has the structure of

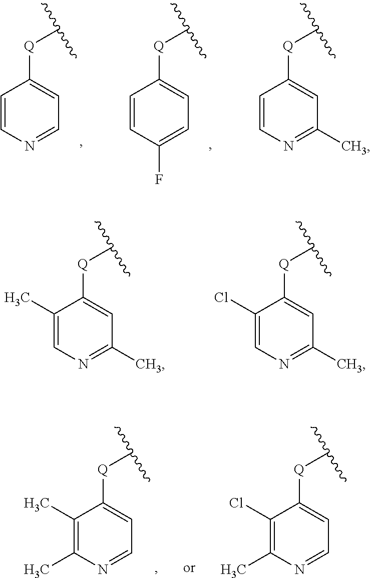

wherein Q is O or NH.

In any one of the embodiments disclosed herein, the structural moiety

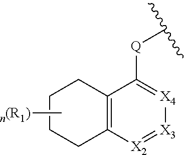

has the structure of

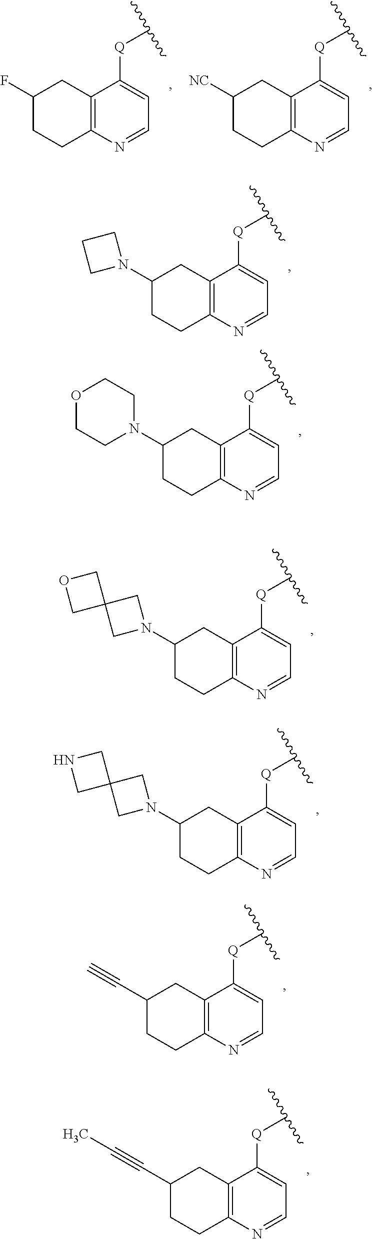

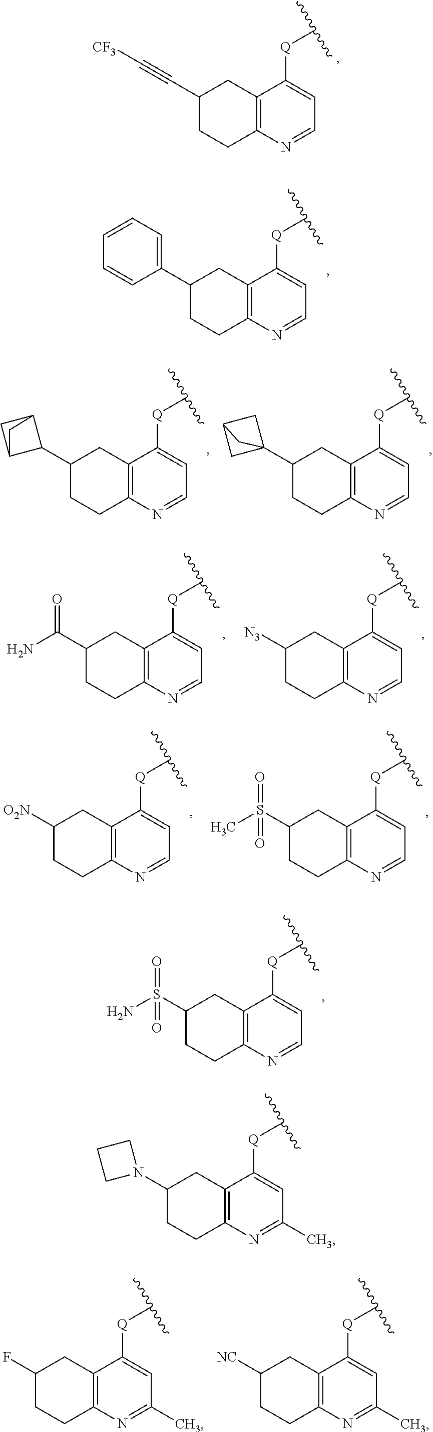

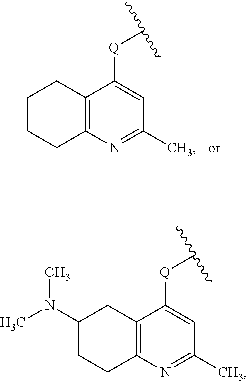

wherein Q is O or NH.

In any one of the embodiments disclosed herein, the compound has the formula of Formula Ia.

In any one of the embodiments disclosed herein, the structural moiety

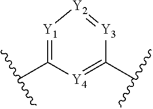

has the structure of

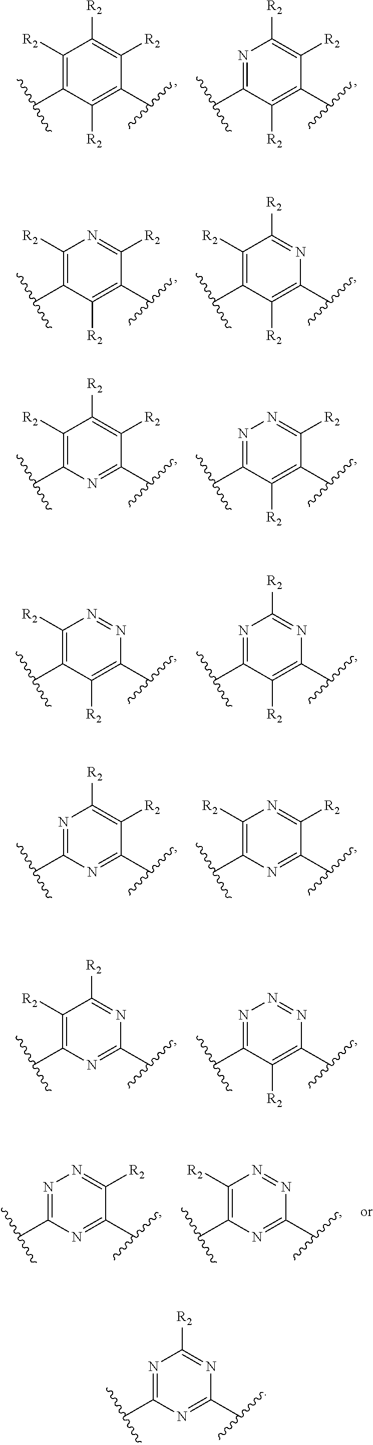

In any one of the embodiments disclosed herein, the structural moiety

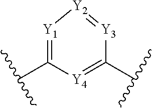

has the structure of

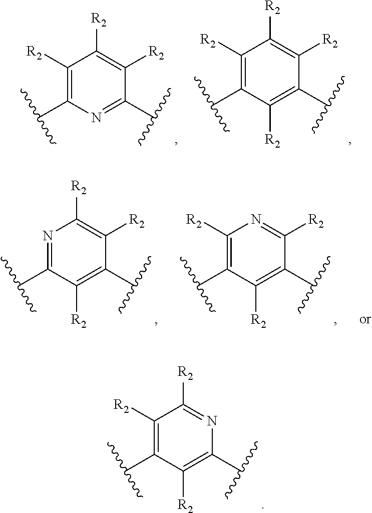

In any one of the embodiments disclosed herein, each occurrence of R₂ is independently H, halogen, CH₃, CF₃, OH, NH₂, —NHCH₃, or —N(CH₃)₂.

In any one of the embodiments disclosed herein, the structural moiety

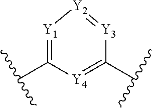

has the structure of

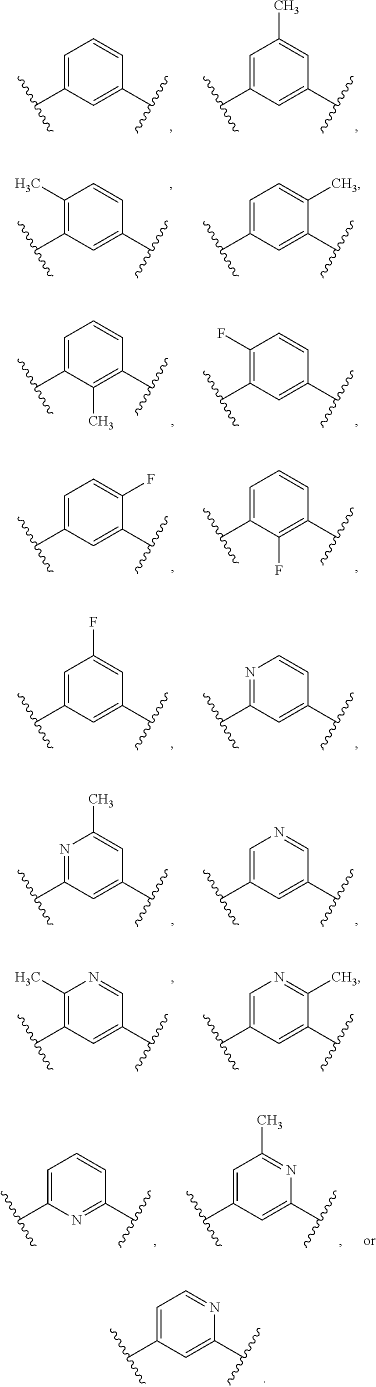

In any one of the embodiments disclosed herein, the structural moiety

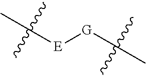

has the structure of

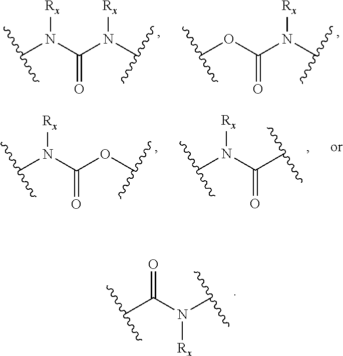

In any one of the embodiments disclosed herein, the structural moiety

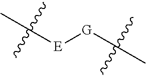

has the structure of

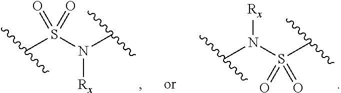

In any one of the embodiments disclosed herein, the structural moiety

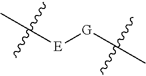

has the structure of

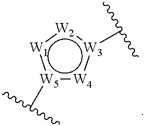

In any one of the embodiments disclosed herein, the structural moiety

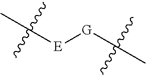

has the structure of

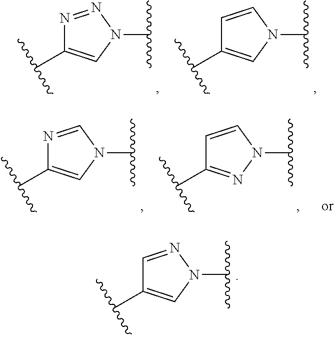 [SMILES: *c1cn(*)nn1 ; *c1ccn(*)c1 ; *c1cn(*)cn1 ; *c1ccn(*)n1 ; *c1cnn(*)c1]

In any one of the embodiments disclosed herein, the structural moiety

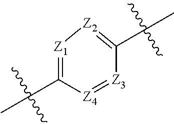

has the structure of

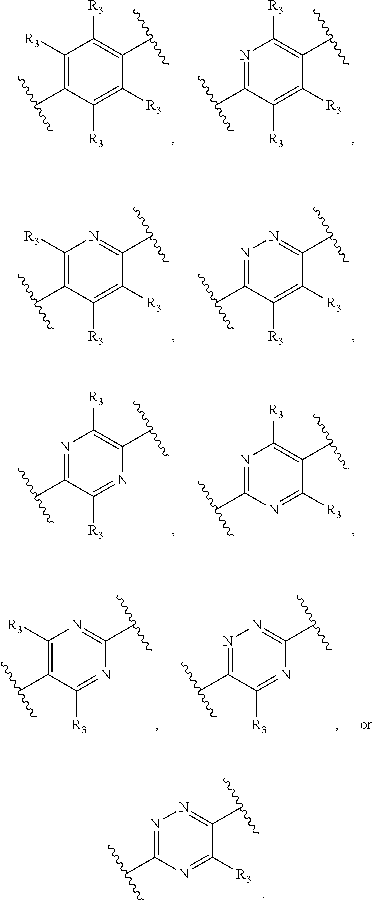

In any one of the embodiments disclosed herein, the structural moiety

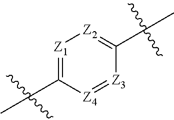

has the structure of

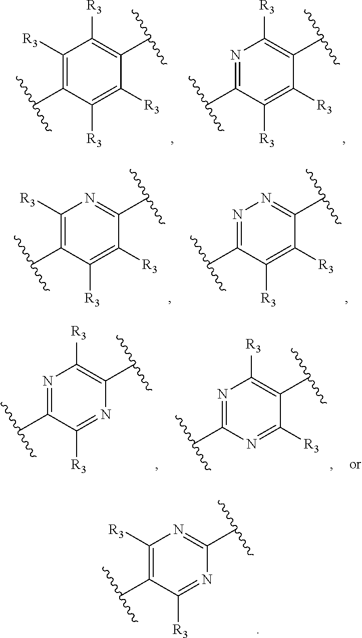

In any one of the embodiments disclosed herein, each occurrence of R₃ is H, halogen, CH₃, CF₃, OH, NH₂, —NHCH₃, or —N(CH₃)₂.

In any one of the embodiments disclosed herein, the structural moiety

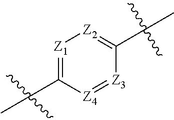

has the structure of

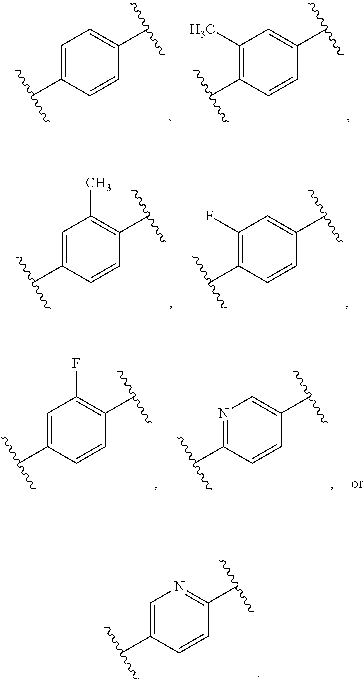

In any one of the embodiments disclosed herein, the structural moiety

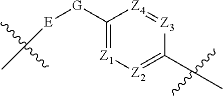

has the structure of

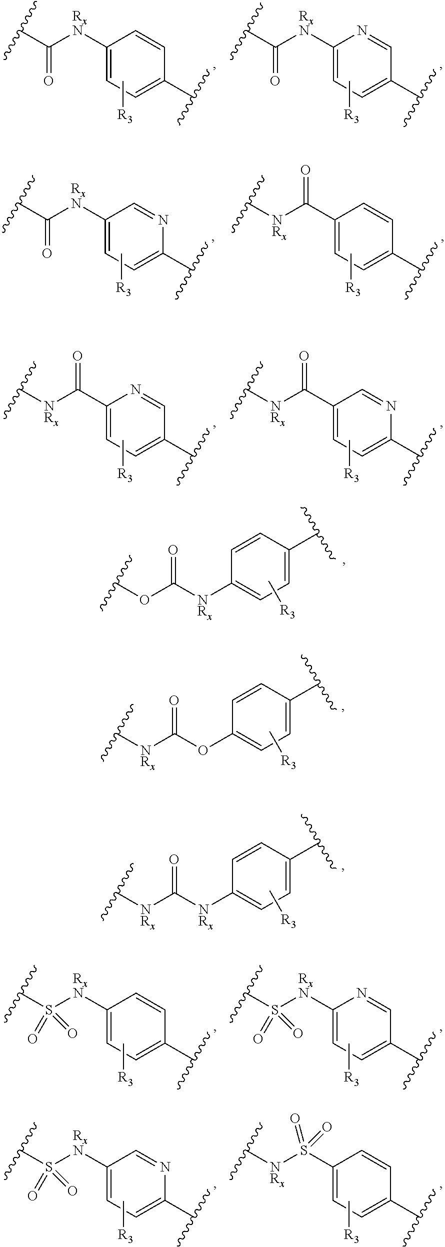

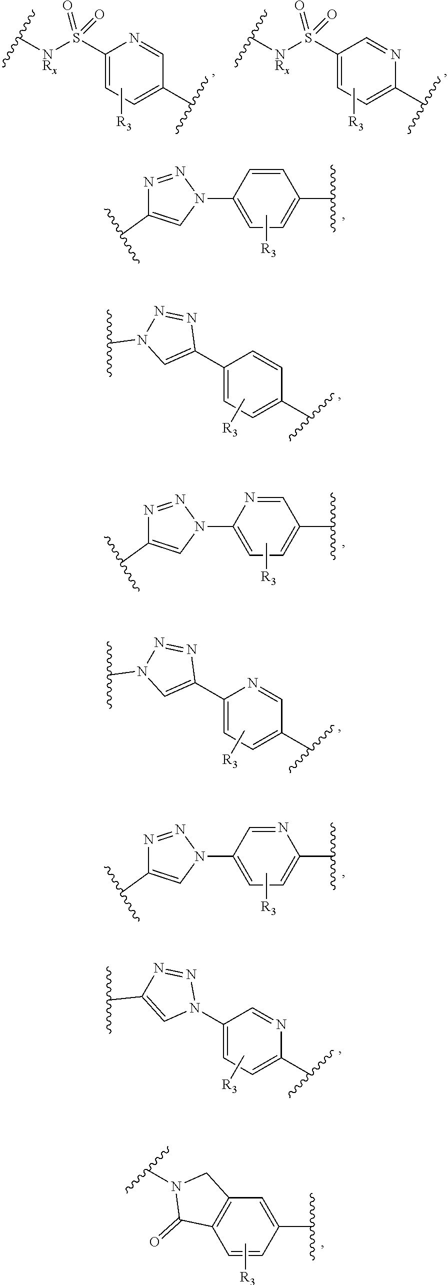

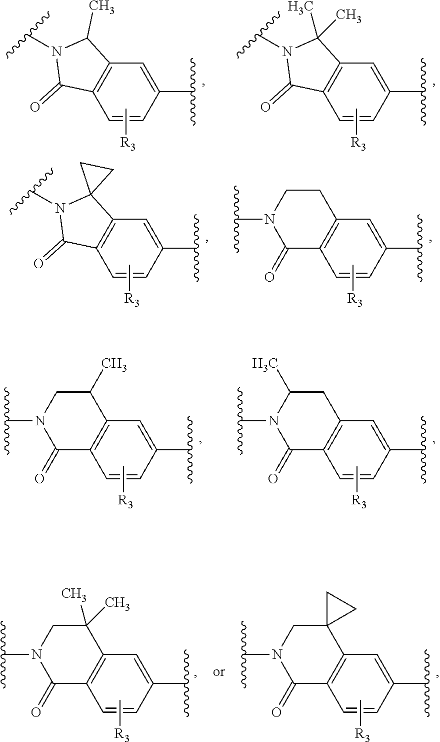

wherein R₃ is H, CH₃, OH, halogen, or NH₂; and wherein Rₓ is H, CH₃, or CH₂CH₃.

In any one of the embodiments disclosed herein, the structural moiety

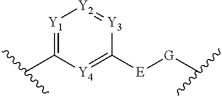

has the structure of

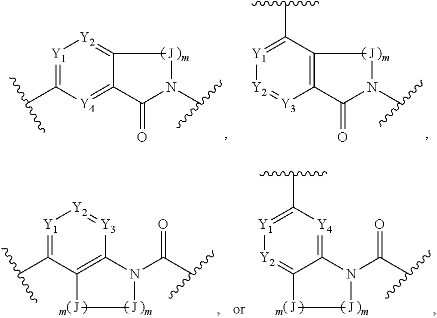

wherein each occurrence of m is independently 1 or 2, J is C(R_{y})₂, and each occurrence of R_{y} is independently H, (C₁-C₆)alkyl, OH, O(C₁-C₆)alkyl, or halogen; or any two R_{y} groups taken together with the carbon atom(s) they are connected to form a (C₃-C₇)cycloalkyl or (C₃-C₇)heterocycloalkyl.

In any one of the embodiments disclosed herein, the structural moiety

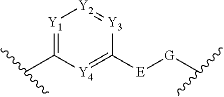

has the structure of

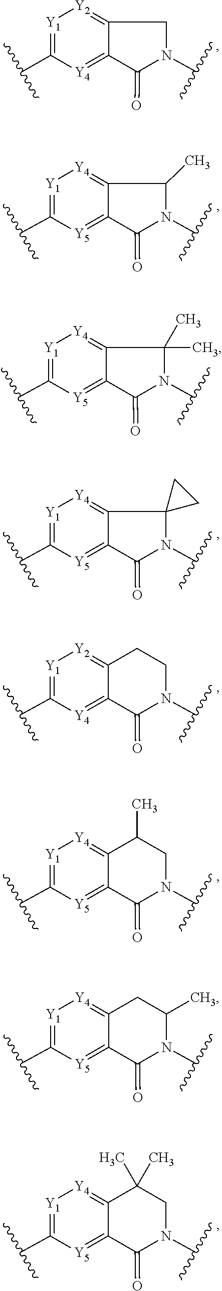

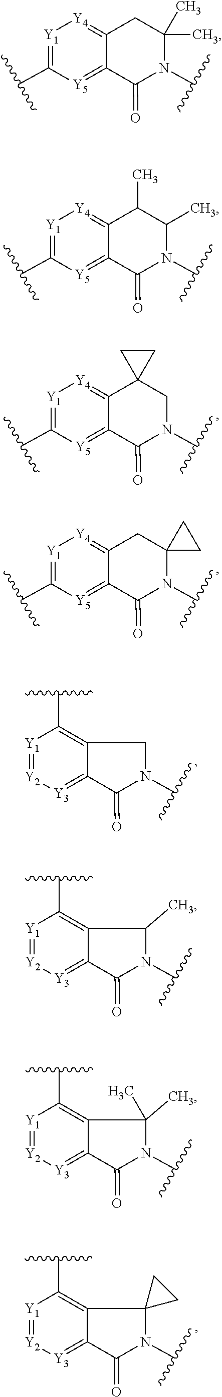

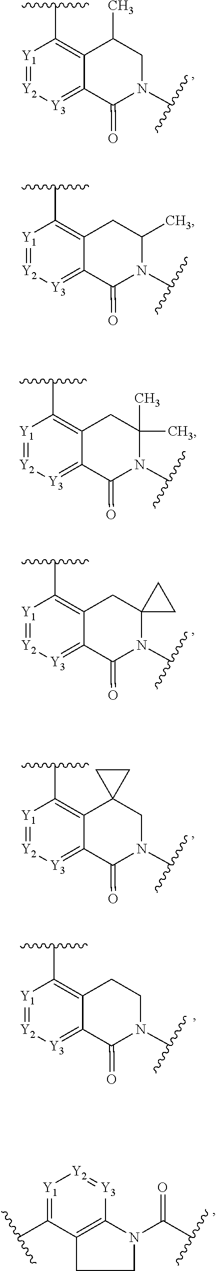

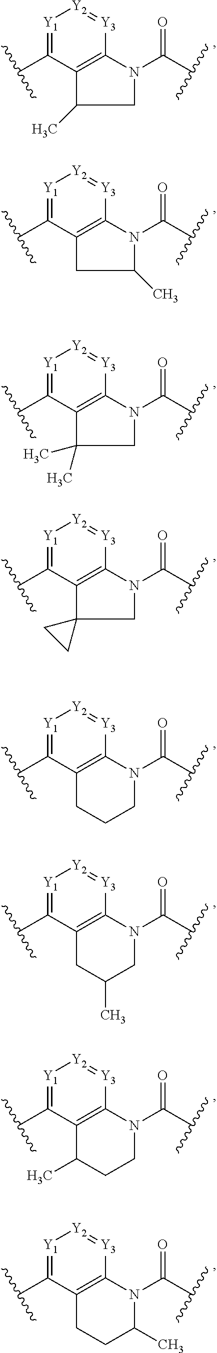

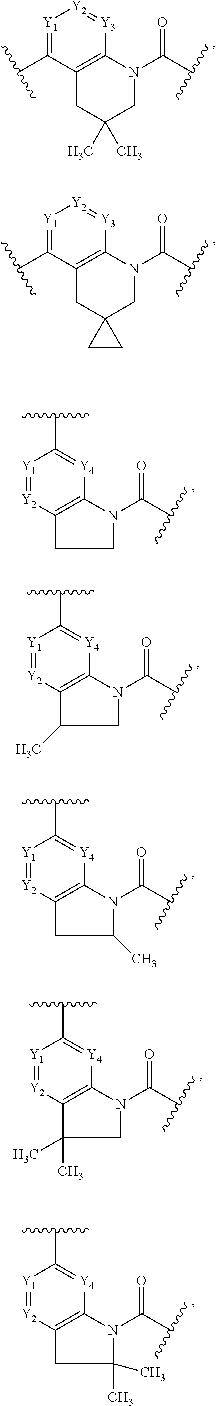

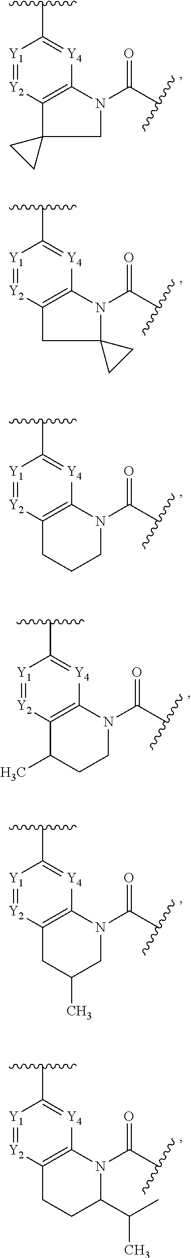

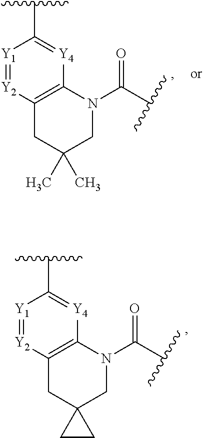

wherein Y₁, Y₂, Y₃, and Y₄ are each independently N, CH, CCH₃, or CF.

In any one of the embodiments disclosed herein, the structural moiety

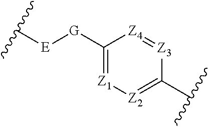

has the structure of

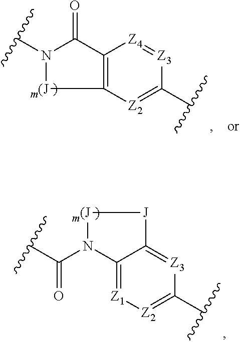

wherein each occurrence of m is independently 1 or 2, J is C(R_{z})₂, and each occurrence of R_{z} is independently H, (C₁-C₆)alkyl, OH, O(C₁-C₆)alkyl, or halogen; or any two R_{z} groups taken together with the carbon atom(s) they are connected to form a (C₃-C₇)cycloalkyl or (C₃-C₇)heterocycloalkyl.

In any one of the embodiments disclosed herein, the structural moiety

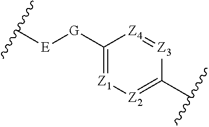

has the structure of

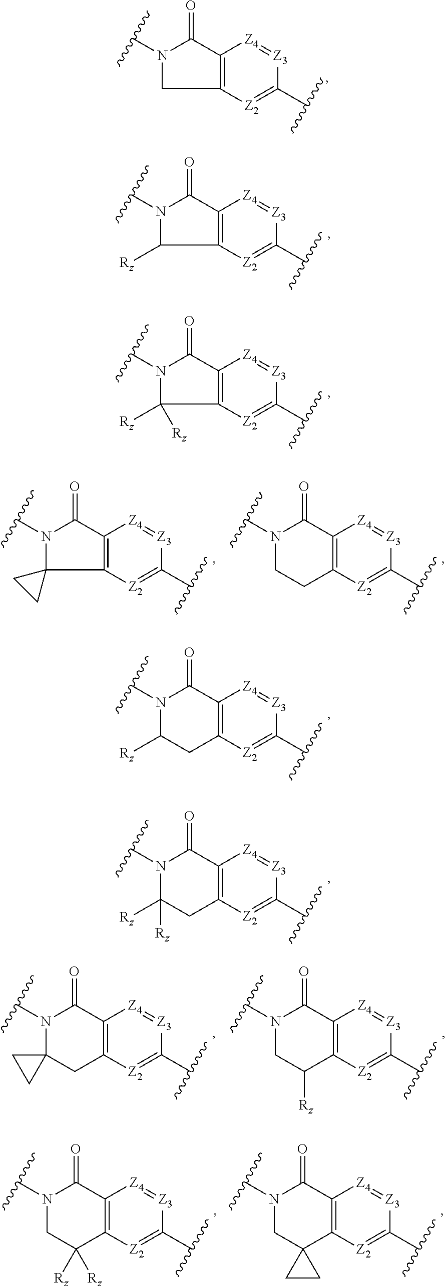

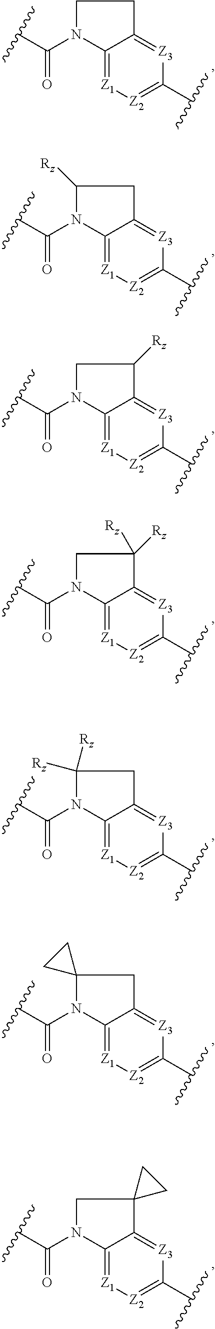

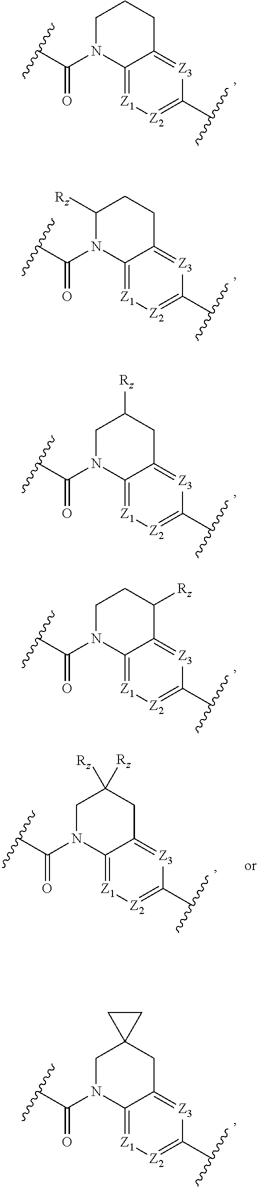

wherein Z₁, Z₂, Z₃, and Z₄ are each independently N, CH, CCH₃, or CF.

In any one of the embodiments disclosed herein, the structural moiety

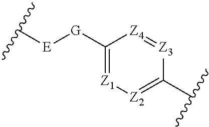

has the structure of

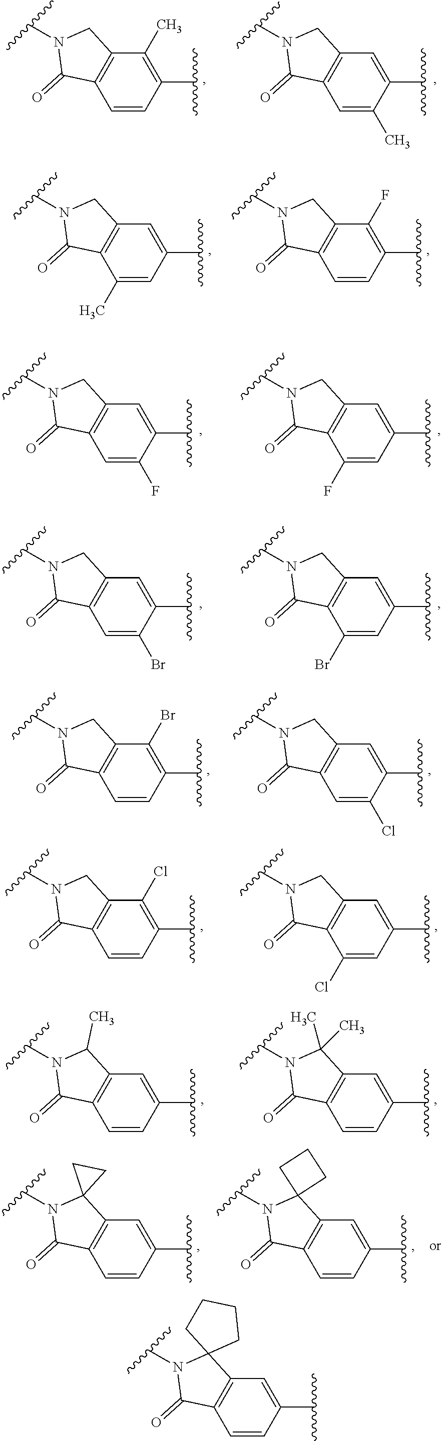

In any one of the embodiments disclosed herein, the compound has the formula of Formula Ib.

In any one of the embodiments disclosed herein, the structural moiety

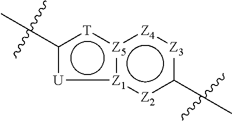

has the structure of

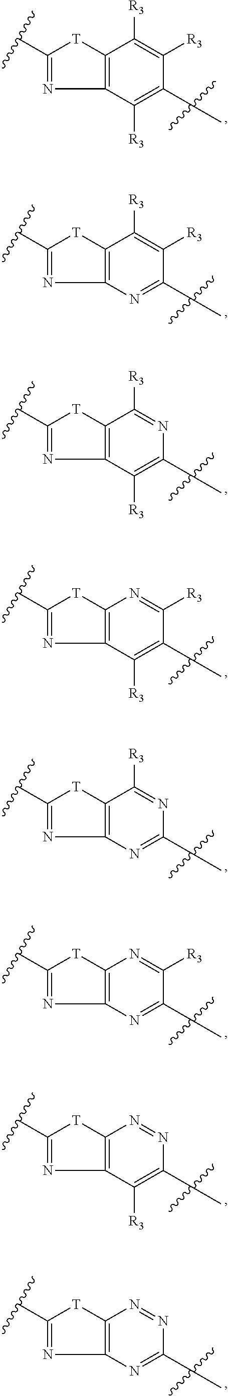

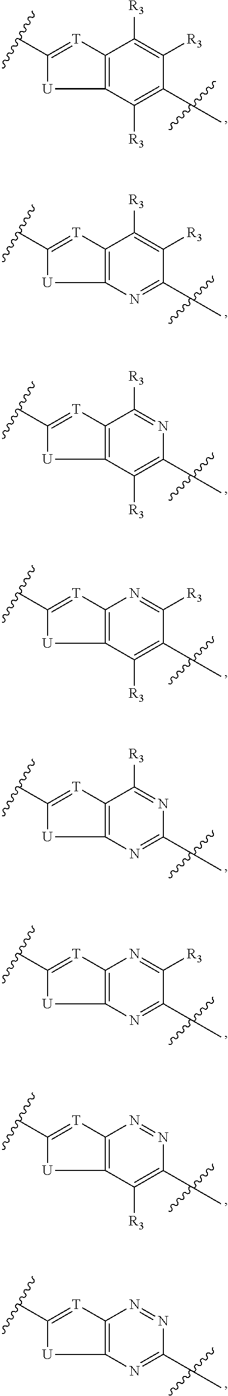

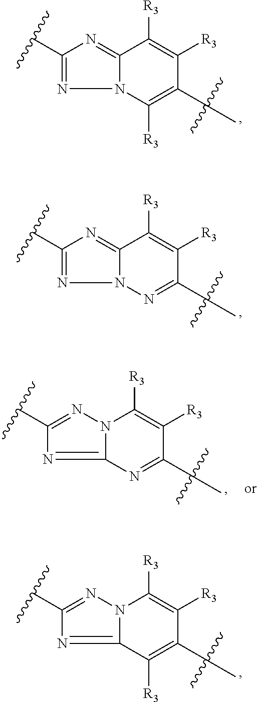

wherein each occurrence of T and U is independently O, N, NRₐ, N(C═O)Rₐ, NC(R_{b})₂OP(═O)(OR_{b})₂, or NSO₂Rₐ where valance permits.

In any one of the embodiments disclosed herein, the structural moiety

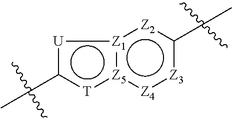

has the structure of

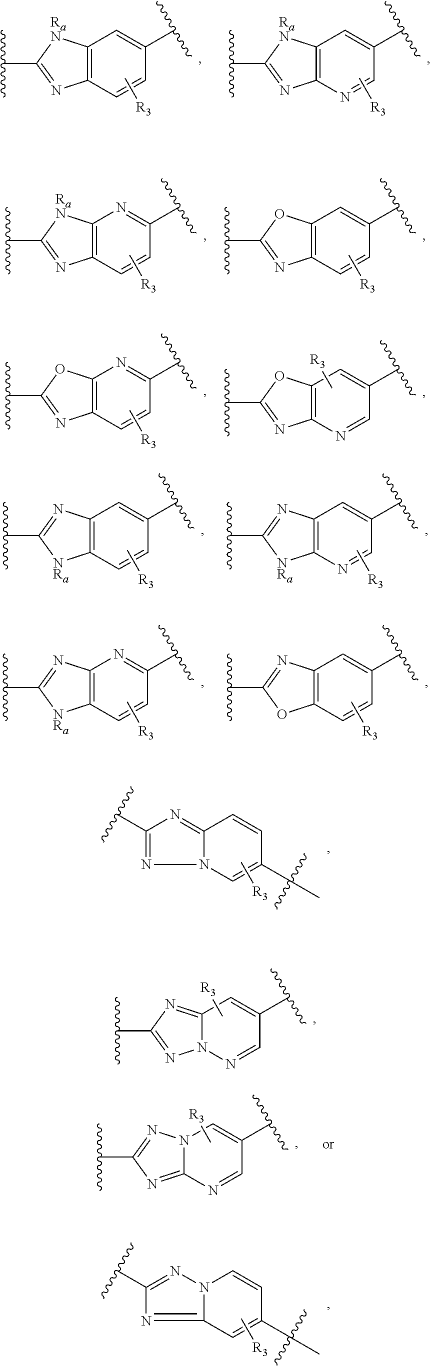

wherein R₃ is H, CH₃, OH, halogen, or NH₂; and wherein Rₐ is H, CH₃, or CH₂CH₃.

In any one of the embodiments disclosed herein, the structural moiety

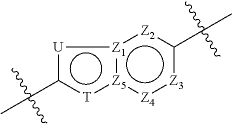

has the structure of

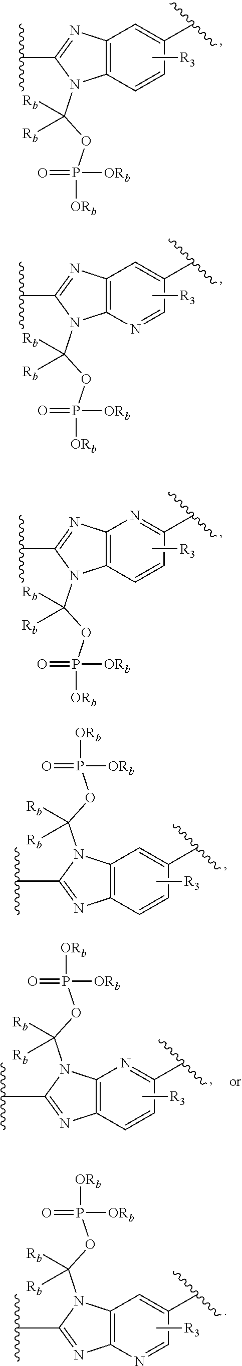

In any one of the embodiments disclosed herein, each occurrence of R_{b} is independently H or (C₁-C₆)alkyl.

In any one of the embodiments disclosed herein, each occurrence of R_{b} is independently H, CH₃, CH₂CH₃, or CH(CH₃)₂.

In any one of the embodiments disclosed herein, the compound has the formula of Formula Ic.

In any one of the embodiments disclosed herein, the structural moiety

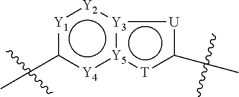

has the structure of

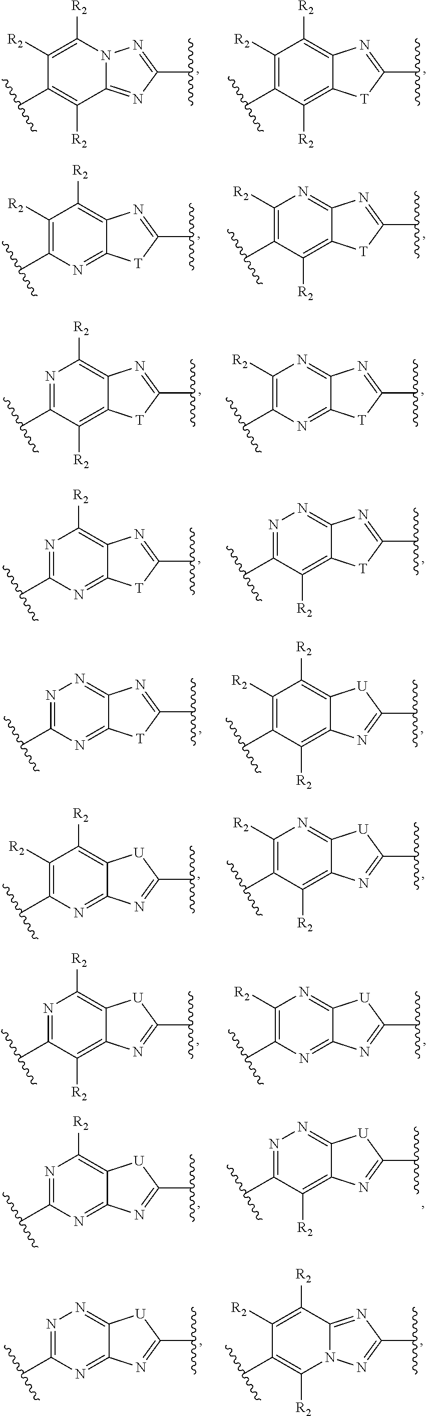

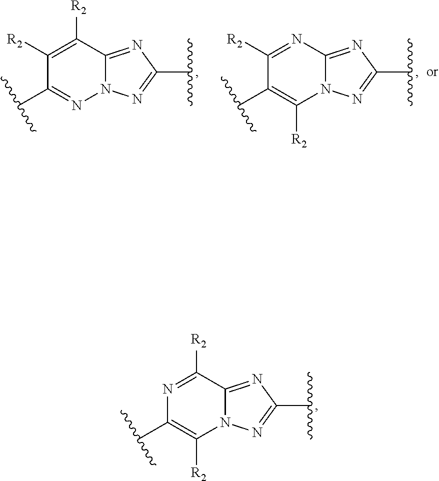

wherein each occurrence of T and U is independently O, N, NRₐ, N(C═O)Rₐ, NC(R_{b})₂OP(═O)(OR_{b})₂, or NSO₂Rₐ where valance permits.

In any one of the embodiments disclosed herein, the structural moiety

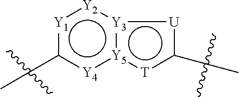

has the structure of

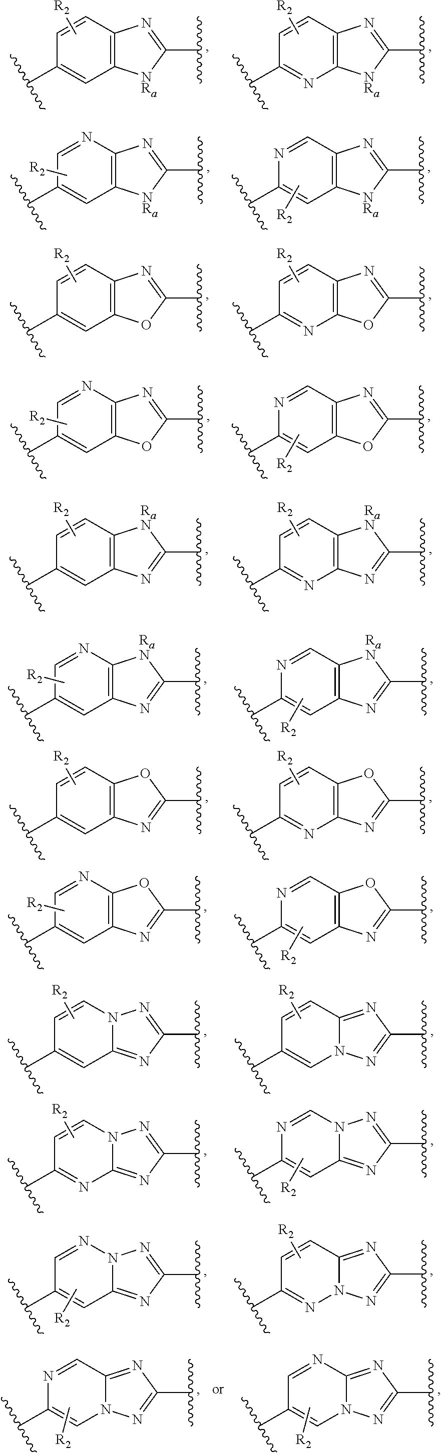

wherein R₂ is H, CH₃, OH, halogen, or NH₂; and wherein Rₐ is H, CH₃, or CH₂CH₃.

In any one of the embodiments disclosed herein, the structural moiety

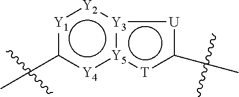

has the structure of

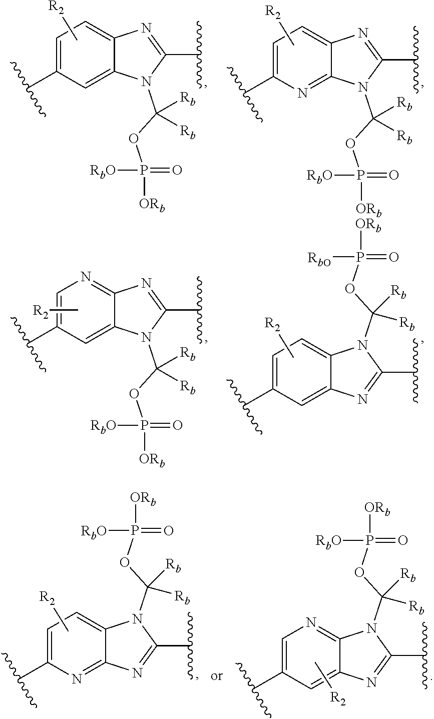

In any one of the embodiments disclosed herein, each occurrence of R_{b} is independently H or (C₁-C₆)alkyl.

In any one of the embodiments disclosed herein, each occurrence of R_{b} is independently H, CH₃, CH₂CH₃, or CH(CH₃)₂.

In any one of the embodiments disclosed herein, each occurrence of R₂ is independently H, CH₃, OH, NH₂, or halogen.

In any one of the embodiments disclosed herein, the structural moiety

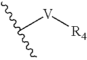

has the structure of

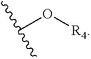

In any one of the embodiments disclosed herein, the structural moiety

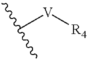

has the structure of

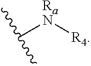

In any one of the embodiments disclosed herein, the structural moiety

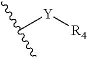

has the structure of

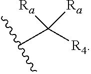

In any one of the embodiments disclosed herein, the V and R₄ of the structural moiety

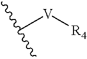

taken together form a (C₄-C₁₀)heterospiroalkyl.

In any one of the embodiments disclosed herein, V is absent.

In any one of the embodiments disclosed herein, R₄ is (C₁-C₆)alkyl,

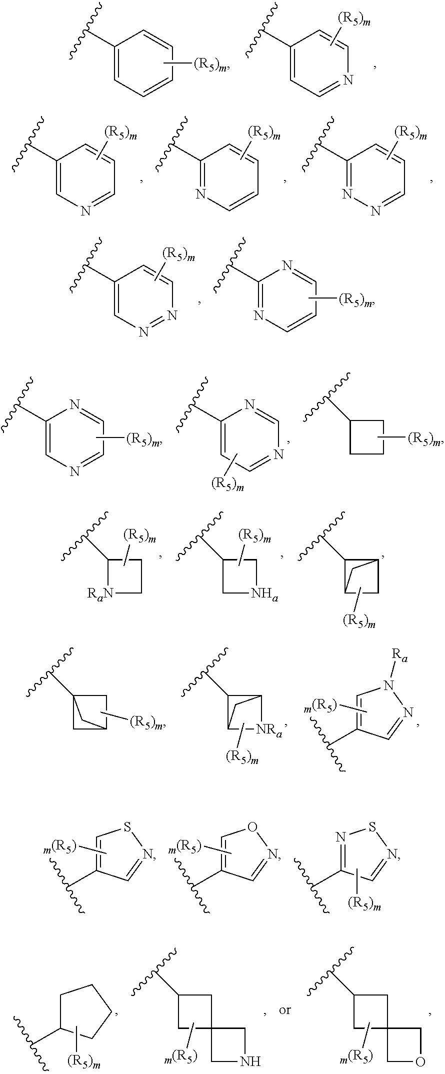

wherein m is an integer from 0-3.

In any one of the embodiments disclosed herein, each occurrence of R₅ is independently H, (C₁-C₆)alkyl, halogen, ORₐ, OH, NH₂, N(Rₐ)CORₐ, CN, CF₃, (C₁-C₆)haloalkyl, or

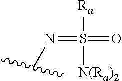

and each occurrence of Rₐ is independently H, (C₂-C₆)alkenyl, or (C₁-C₆)alkyl.

In any one of the embodiments disclosed herein, the structural moiety

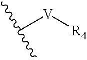

has the structure of

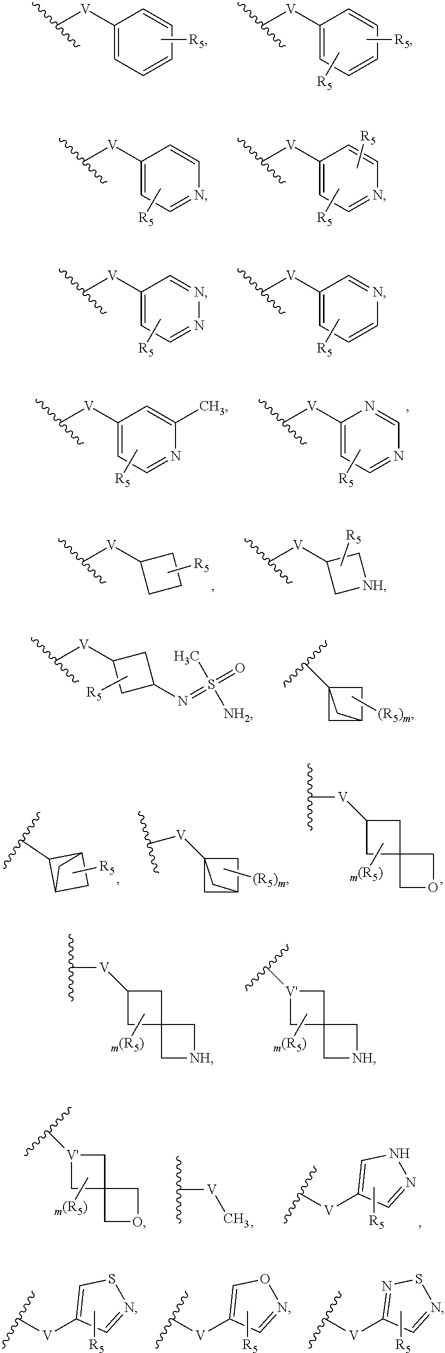

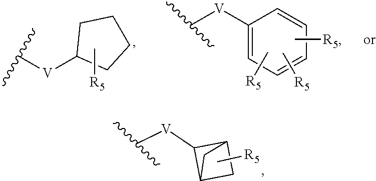

wherein V is C(Rₐ)₂, O, NRₐ, N(C═O)Rₐ, or NSO₂Rₐ and V′ is CRₐ or N.

In any one of the embodiments disclosed herein, each occurrence of R₅ is independently H, CH₃, halogen, OH, CN,

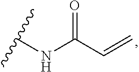 [SMILES: *NC(=O)C=C]

CF₃, (C₁-C₆)haloalkyl, or NH₂.

In any one of the embodiments disclosed herein, each occurrence of Rₐ is independently H, (C₂-C₆)alkenyl, or (C₁-C₆)alkyl.

In any one of the embodiments disclosed herein, each occurrence of Rₐ is H, CH₃, or CH₂CH₃.

In any one of the embodiments disclosed herein, the structural moiety

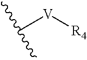

has the structure of

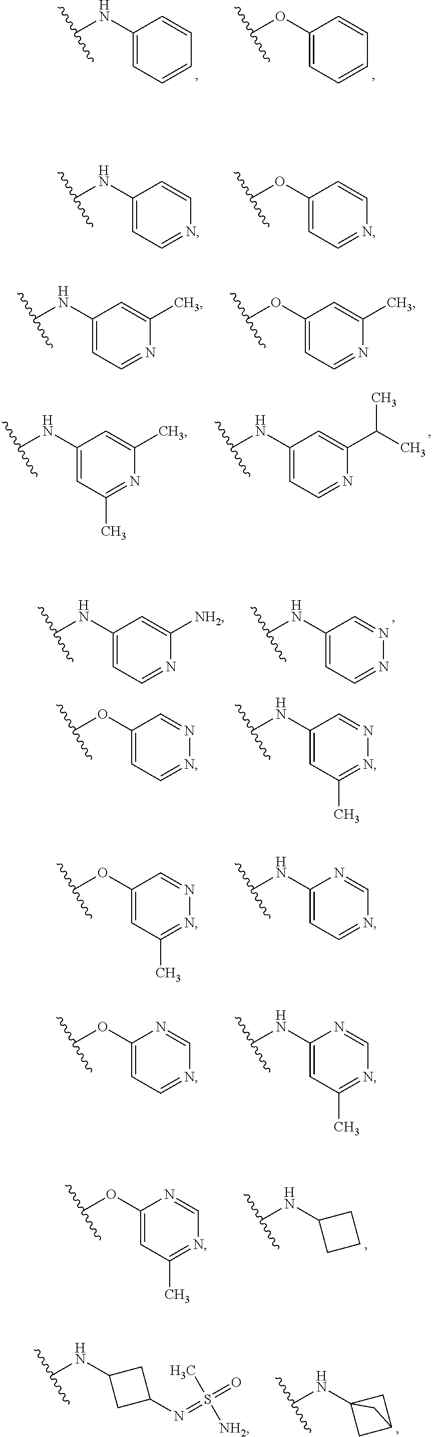

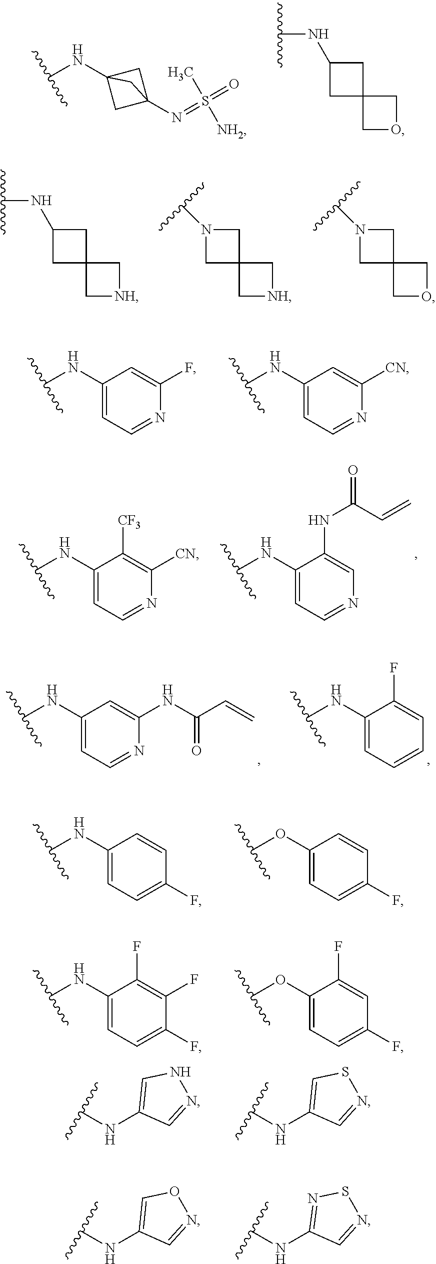

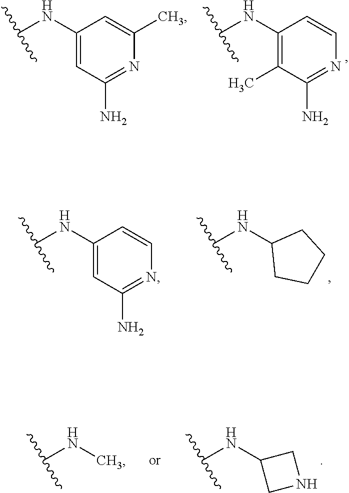

In any one of the embodiments disclosed herein, the structural moiety

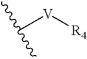

has the structure of

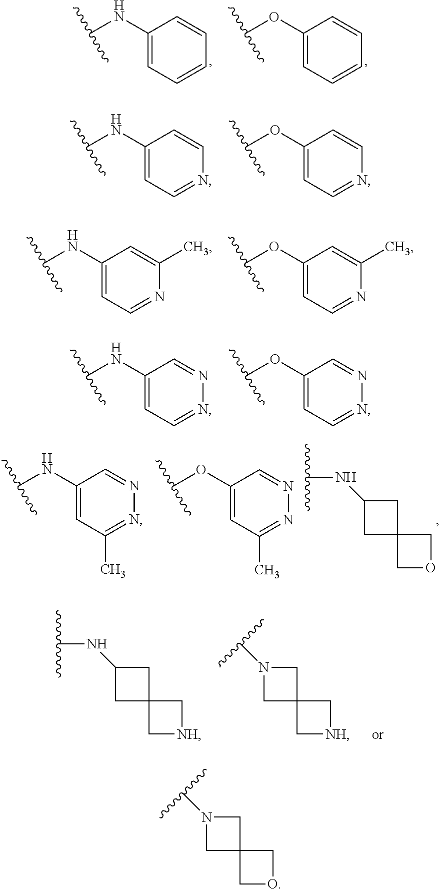

In any one of the embodiments disclosed herein, the compound of Formula Ia has the structure of

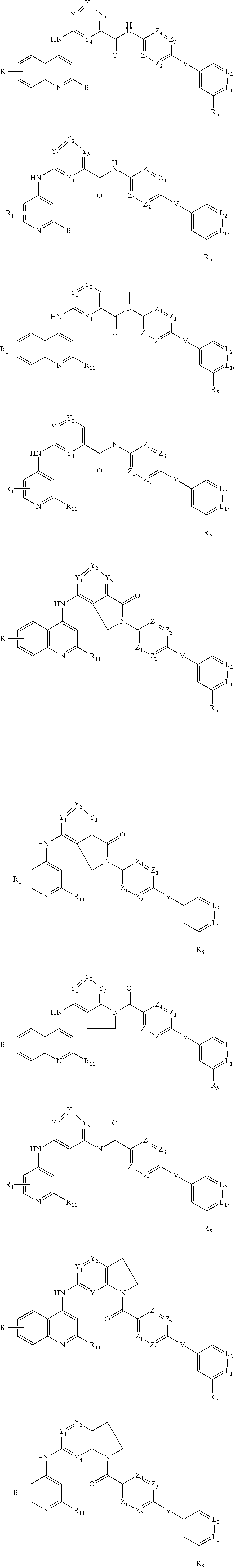

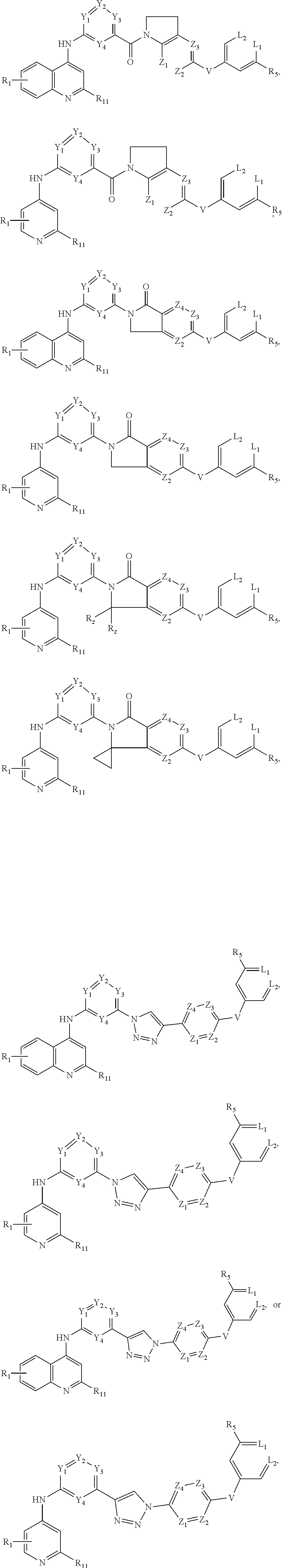

wherein R₁ is H, (C₁-C₆)alkyl, N(Rₐ)₂, (C₃-C₇)heterocycloalkyl, or halogen; R₅ and R₁₁ are each independently H or CH₃; Y₁, Y₂, Y₃, Y₄, Z₁, Z₂, Z₃, Z₄, L₁, and L₂ are each independently CH or N; and V is NH or O.

In any one of the embodiments disclosed herein, R₁ is H, F, Cl, Br, CH₃, CH₂CH₃, CH(CH₃)₂, NH₂, NMe₂,

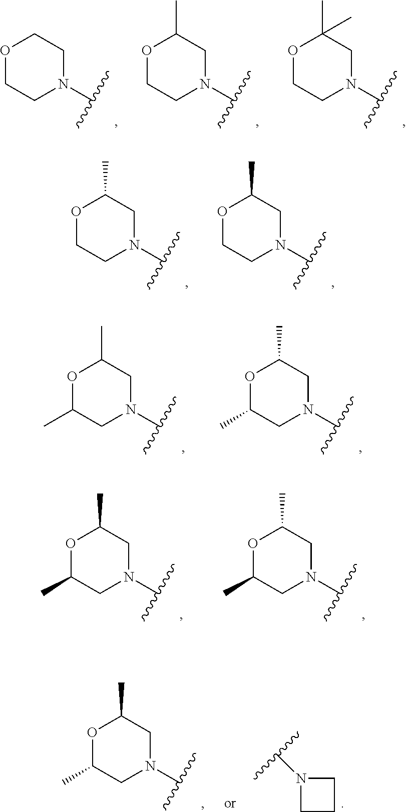

In any one of the embodiments disclosed herein, the compound of Formula Ib has the structure

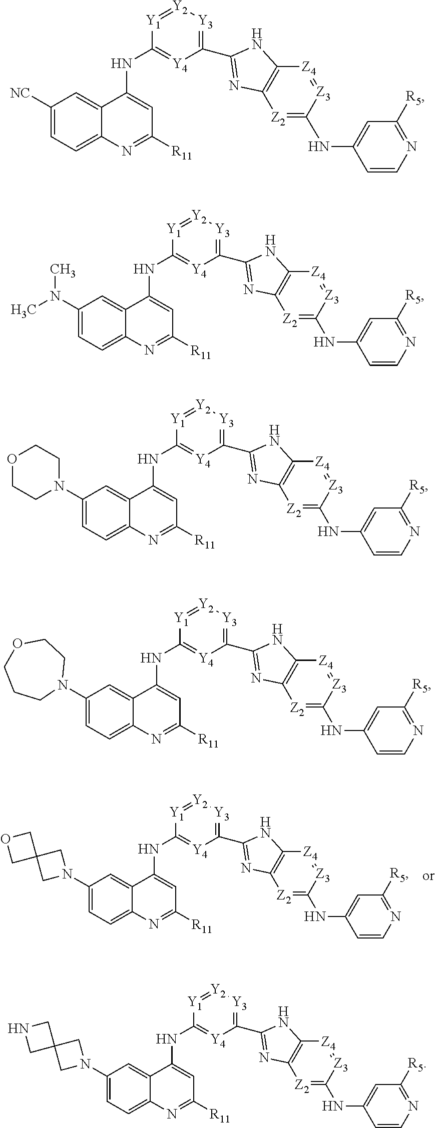

wherein R₁₁ and R₅ are each independently H or CH₃; and Y₁, Y₂, Y₃, Y₄, Z₂, Z₃, and Z₄ are each independently CH or N.

In any one of the embodiments disclosed herein, the compound of Formula Ib has the structure

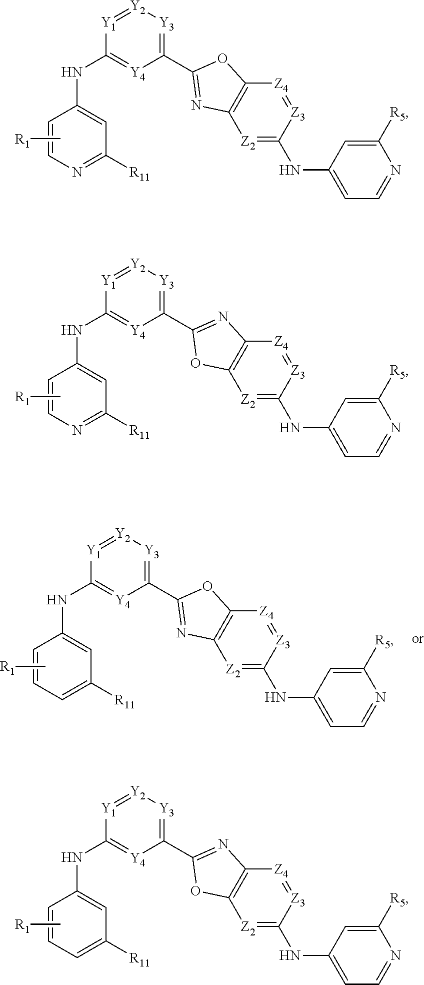

wherein R₁, R₅, and R₁₁ are each independently H, halogen, or CH₃; and Y₁, Y₂, Y₃, Y₄, Z₂, Z₃, and Z₄ are each independently CH or N.

In any one of the embodiments disclosed herein, the compound of Formula Ic is

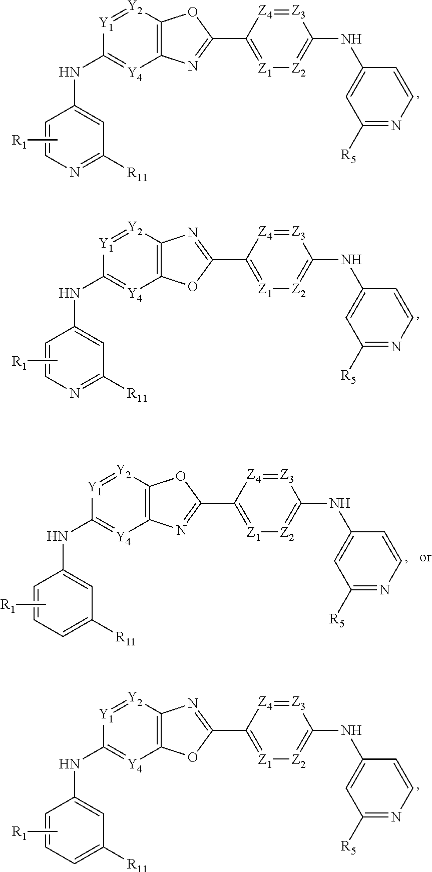

wherein R₁, R₅, and R₁₁ are each independently H, halogen, or CH₃; and Y₁, Y₂, Y₄, Z₁, Z₂, Z₃, and Z₄ are each independently CH or N.

In any one of the embodiments disclosed herein, the compound of Formula Ia is

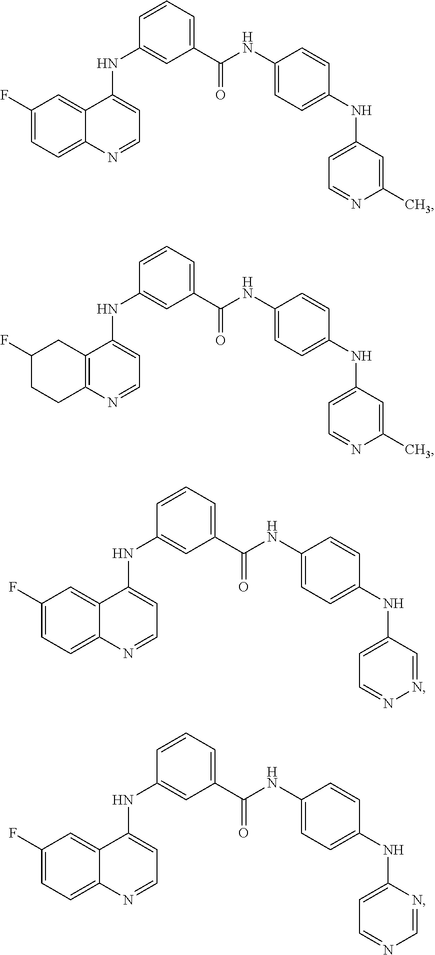 [SMILES: Cc1cc(Nc2ccc(NC(=O)c3cccc(Nc4ccnc5ccc(F)cc45)c3)cc2)ccn1 ; Cc1cc(Nc2ccc(NC(=O)c3cccc(Nc4ccnc5c4CC(F)CC5)c3)cc2)ccn1 ; O=C(Nc1ccc(Nc2ccnnc2)cc1)c1cccc(Nc2ccnc3ccc(F)cc23)c1 ; O=C(Nc1ccc(Nc2ccncn2)cc1)c1cccc(Nc2ccnc3ccc(F)cc23)c1]

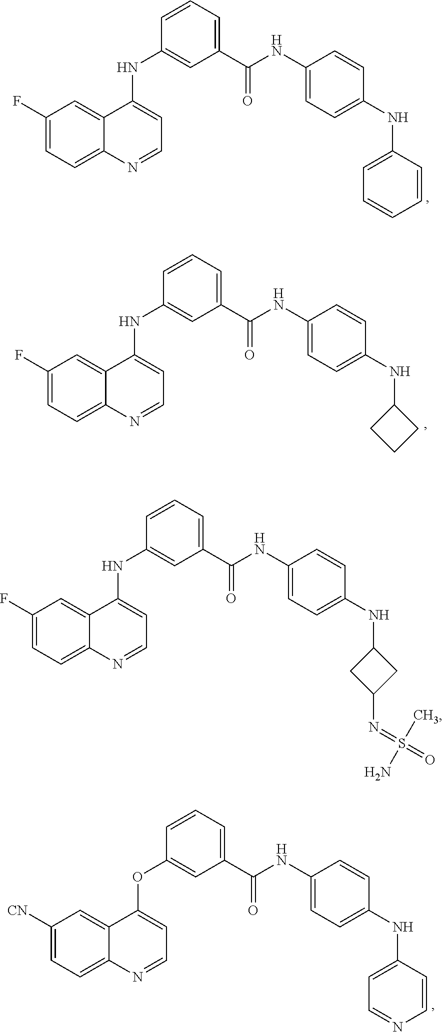

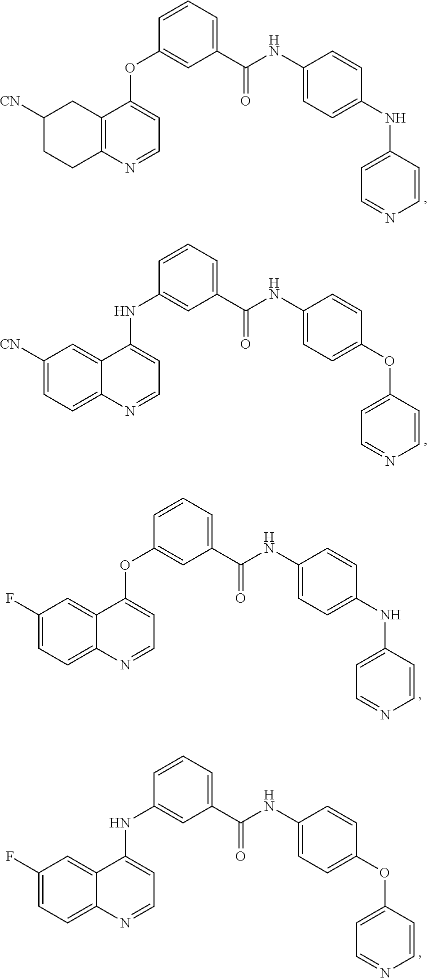

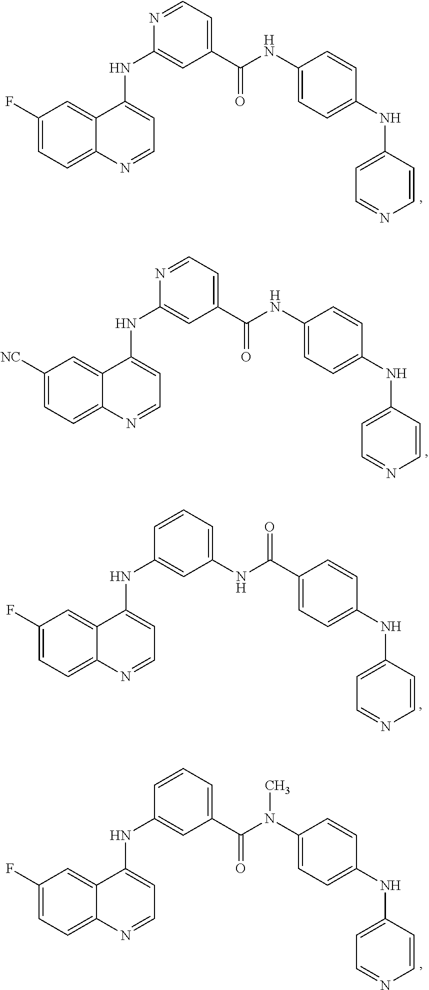 [SMILES: O=C(Nc1ccc(Nc2ccncc2)cc1)c1ccnc(Nc2ccnc3ccc(F)cc23)c1 ; N#Cc1ccc2nccc(Nc3cc(C(=O)Nc4ccc(Nc5ccncc5)cc4)ccn3)c2c1 ; O=C(Nc1cccc(Nc2ccnc3ccc(F)cc23)c1)c1ccc(Nc2ccncc2)cc1 ; CN(C(=O)c1cccc(Nc2ccnc3ccc(F)cc23)c1)c1ccc(Nc2ccncc2)cc1]

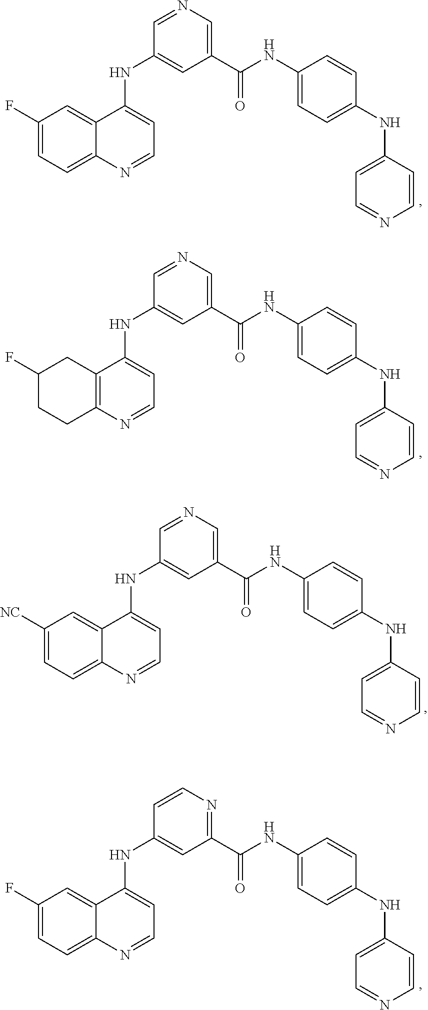 [SMILES: O=C(Nc1ccc(Nc2ccncc2)cc1)c1cncc(Nc2ccnc3ccc(F)cc23)c1 ; O=C(Nc1ccc(Nc2ccncc2)cc1)c1cncc(Nc2ccnc3c2CC(F)CC3)c1 ; N#Cc1ccc2nccc(Nc3cncc(C(=O)Nc4ccc(Nc5ccncc5)cc4)c3)c2c1 ; O=C(Nc1ccc(Nc2ccncc2)cc1)c1cc(Nc2ccnc3ccc(F)cc23)ccn1]

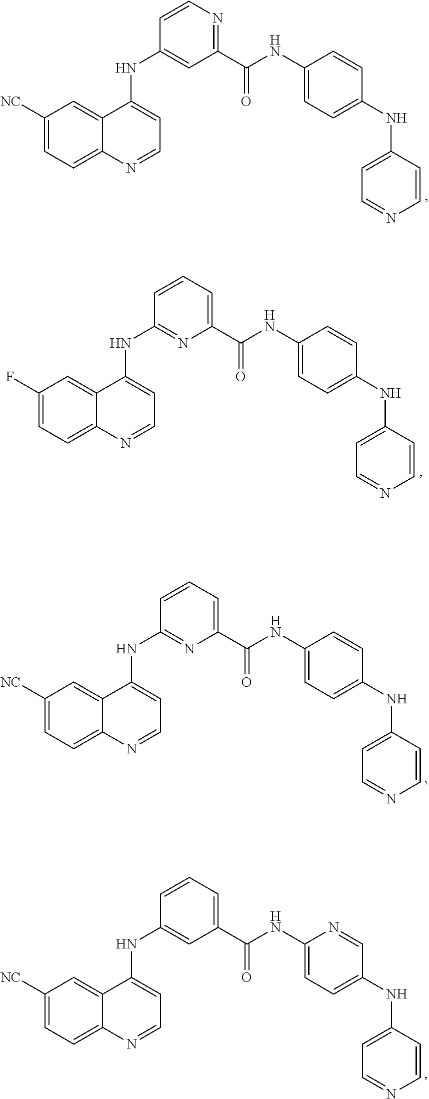 [SMILES: N#Cc1ccc2nccc(Nc3ccnc(C(=O)Nc4ccc(Nc5ccncc5)cc4)c3)c2c1 ; O=C(Nc1ccc(Nc2ccncc2)cc1)c1cccc(Nc2ccnc3ccc(F)cc23)n1 ; N#Cc1ccc2nccc(Nc3cccc(C(=O)Nc4ccc(Nc5ccncc5)cc4)n3)c2c1 ; N#Cc1ccc2nccc(Nc3cccc(C(=O)Nc4ccc(Nc5ccncc5)cn4)c3)c2c1]

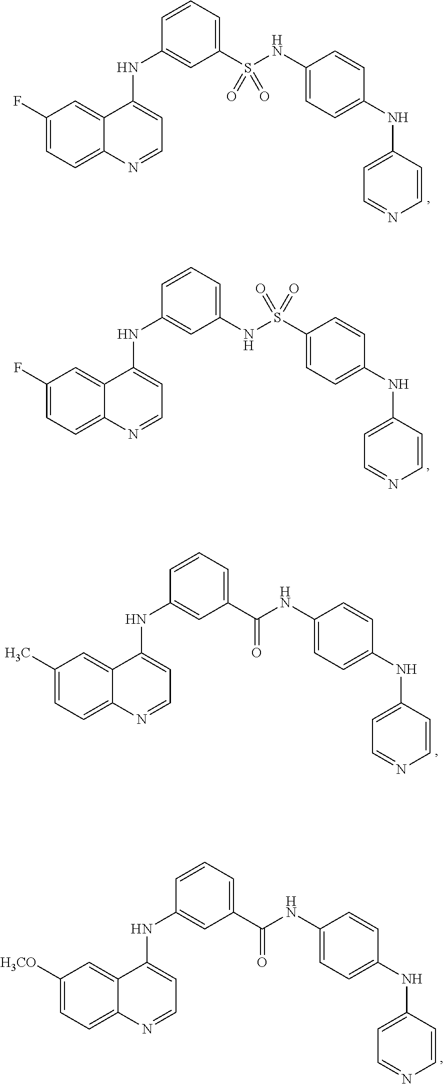 [SMILES: O=S(=O)(Nc1ccc(Nc2ccncc2)cc1)c1cccc(Nc2ccnc3ccc(F)cc23)c1 ; O=S(=O)(Nc1cccc(Nc2ccnc3ccc(F)cc23)c1)c1ccc(Nc2ccncc2)cc1 ; Cc1ccc2nccc(Nc3cccc(C(=O)Nc4ccc(Nc5ccncc5)cc4)c3)c2c1 ; COc1ccc2nccc(Nc3cccc(C(=O)Nc4ccc(Nc5ccncc5)cc4)c3)c2c1]

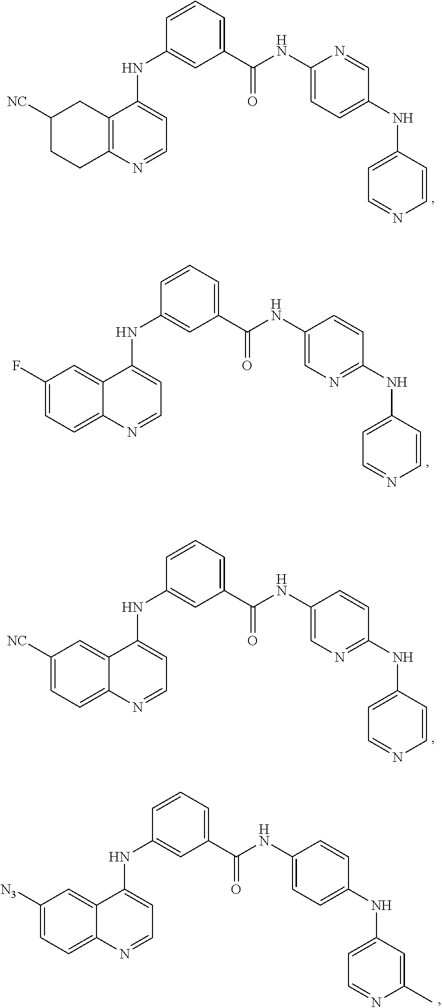 [SMILES: N#CC1CCc2nccc(Nc3cccc(C(=O)Nc4ccc(Nc5ccncc5)cn4)c3)c2C1 ; O=C(Nc1ccc(Nc2ccncc2)nc1)c1cccc(Nc2ccnc3ccc(F)cc23)c1 ; N#Cc1ccc2nccc(Nc3cccc(C(=O)Nc4ccc(Nc5ccncc5)nc4)c3)c2c1 ; Cc1cc(Nc2ccc(NC(=O)c3cccc(Nc4ccnc5ccc(N=[N+]=[N-])cc45)c3)cc2)ccn1]

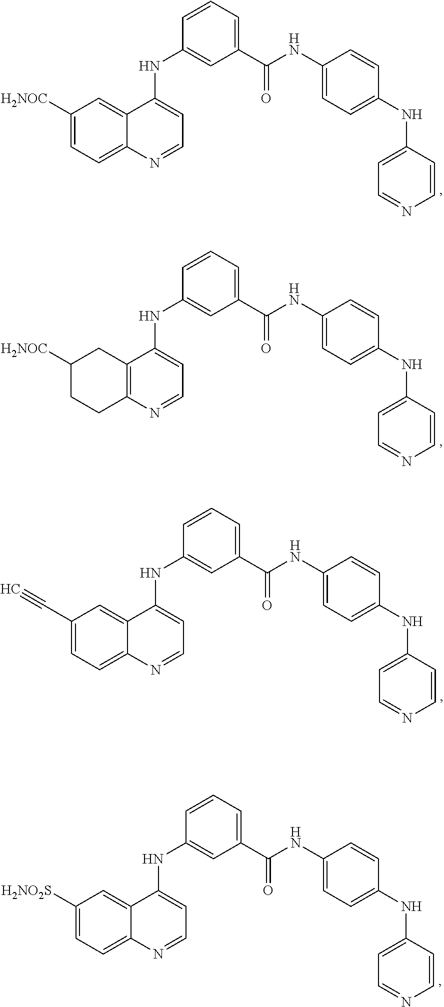 [SMILES: NC(=O)c1ccc2nccc(Nc3cccc(C(=O)Nc4ccc(Nc5ccncc5)cc4)c3)c2c1 ; NC(=O)C1CCc2nccc(Nc3cccc(C(=O)Nc4ccc(Nc5ccncc5)cc4)c3)c2C1 ; C#Cc1ccc2nccc(Nc3cccc(C(=O)Nc4ccc(Nc5ccncc5)cc4)c3)c2c1 ; NS(=O)(=O)c1ccc2nccc(Nc3cccc(C(=O)Nc4ccc(Nc5ccncc5)cc4)c3)c2c1]

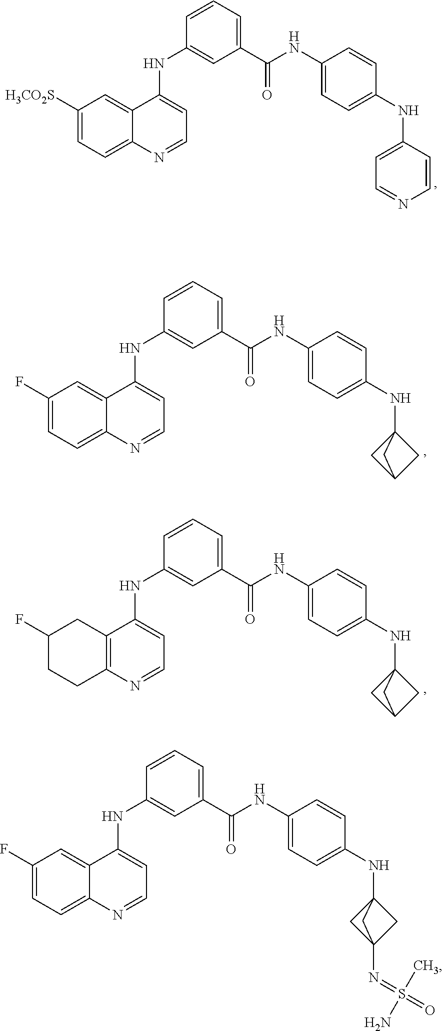

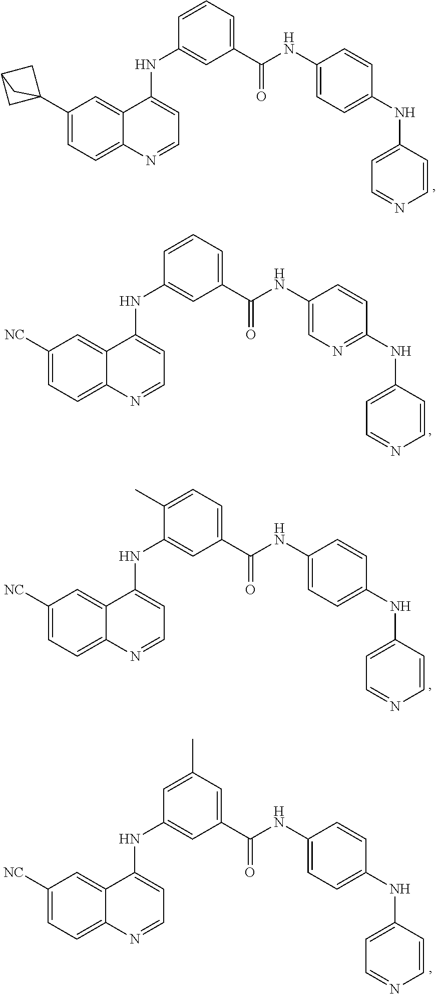 [SMILES: O=C(Nc1ccc(Nc2ccncc2)cc1)c1cccc(Nc2ccnc3ccc(C45CC(C4)C5)cc23)c1 ; N#Cc1ccc2nccc(Nc3cccc(C(=O)Nc4ccc(Nc5ccncc5)nc4)c3)c2c1 ; Cc1ccc(C(=O)Nc2ccc(Nc3ccncc3)cc2)cc1Nc1ccnc2ccc(C#N)cc12 ; Cc1cc(Nc2ccnc3ccc(C#N)cc23)cc(C(=O)Nc2ccc(Nc3ccncc3)cc2)c1]

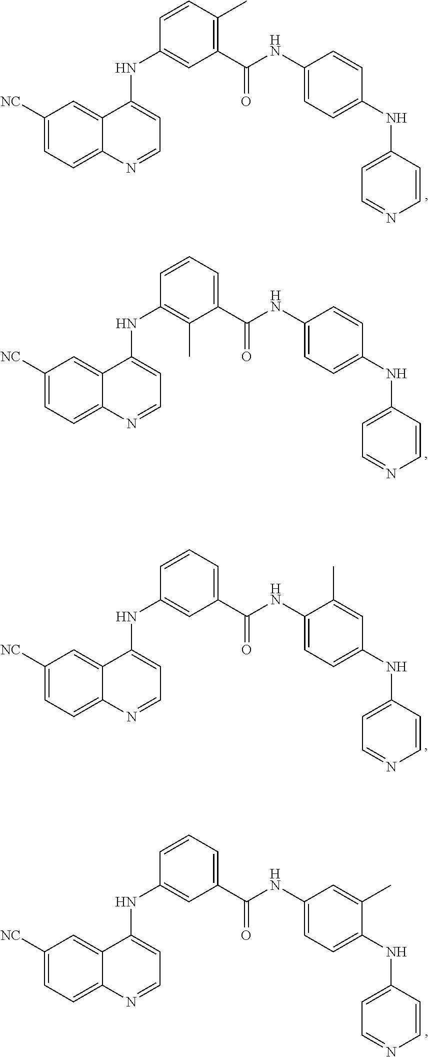 [SMILES: Cc1ccc(Nc2ccnc3ccc(C#N)cc23)cc1C(=O)Nc1ccc(Nc2ccncc2)cc1 ; Cc1c(Nc2ccnc3ccc(C#N)cc23)cccc1C(=O)Nc1ccc(Nc2ccncc2)cc1 ; Cc1cc(Nc2ccncc2)ccc1NC(=O)c1cccc(Nc2ccnc3ccc(C#N)cc23)c1 ; Cc1cc(NC(=O)c2cccc(Nc3ccnc4ccc(C#N)cc34)c2)ccc1Nc1ccncc1]

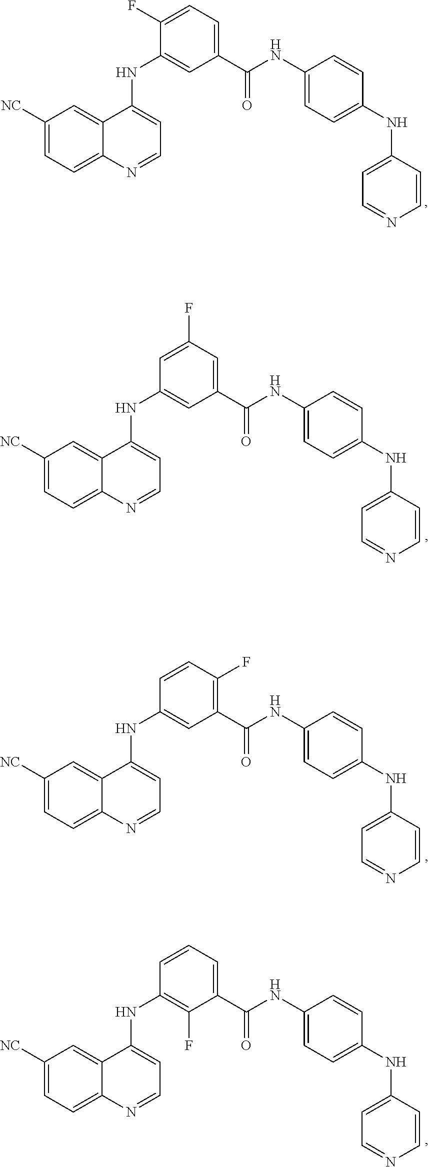 [SMILES: N#Cc1ccc2nccc(Nc3cc(C(=O)Nc4ccc(Nc5ccncc5)cc4)ccc3F)c2c1 ; N#Cc1ccc2nccc(Nc3cc(F)cc(C(=O)Nc4ccc(Nc5ccncc5)cc4)c3)c2c1 ; N#Cc1ccc2nccc(Nc3ccc(F)c(C(=O)Nc4ccc(Nc5ccncc5)cc4)c3)c2c1 ; N#Cc1ccc2nccc(Nc3cccc(C(=O)Nc4ccc(Nc5ccncc5)cc4)c3F)c2c1]

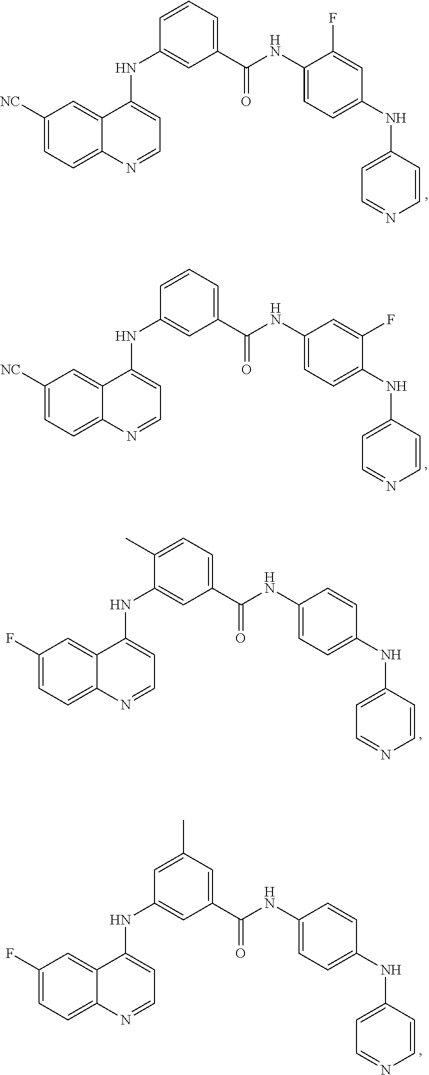 [SMILES: N#Cc1ccc2nccc(Nc3cccc(C(=O)Nc4ccc(Nc5ccncc5)cc4F)c3)c2c1 ; N#Cc1ccc2nccc(Nc3cccc(C(=O)Nc4ccc(Nc5ccncc5)c(F)c4)c3)c2c1 ; Cc1ccc(C(=O)Nc2ccc(Nc3ccncc3)cc2)cc1Nc1ccnc2ccc(F)cc12 ; Cc1cc(Nc2ccnc3ccc(F)cc23)cc(C(=O)Nc2ccc(Nc3ccncc3)cc2)c1]

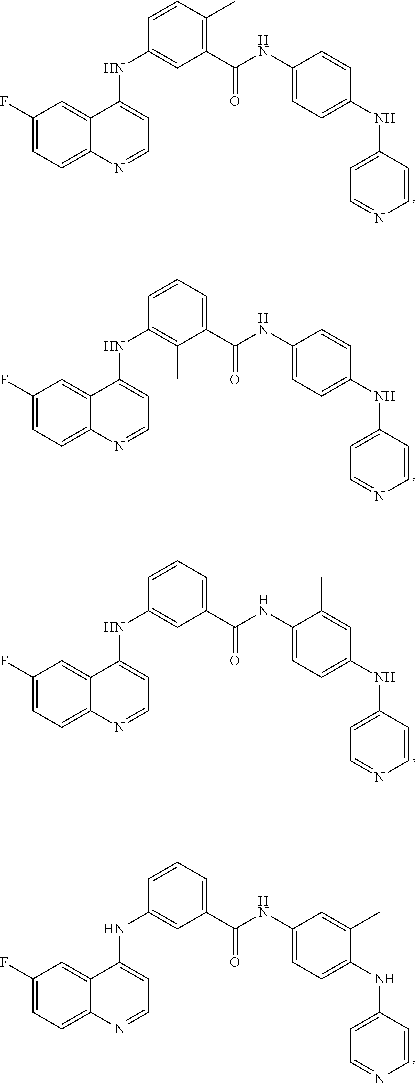 [SMILES: Cc1ccc(Nc2ccnc3ccc(F)cc23)cc1C(=O)Nc1ccc(Nc2ccncc2)cc1 ; Cc1c(Nc2ccnc3ccc(F)cc23)cccc1C(=O)Nc1ccc(Nc2ccncc2)cc1 ; Cc1cc(Nc2ccncc2)ccc1NC(=O)c1cccc(Nc2ccnc3ccc(F)cc23)c1 ; Cc1cc(NC(=O)c2cccc(Nc3ccnc4ccc(F)cc34)c2)ccc1Nc1ccncc1]

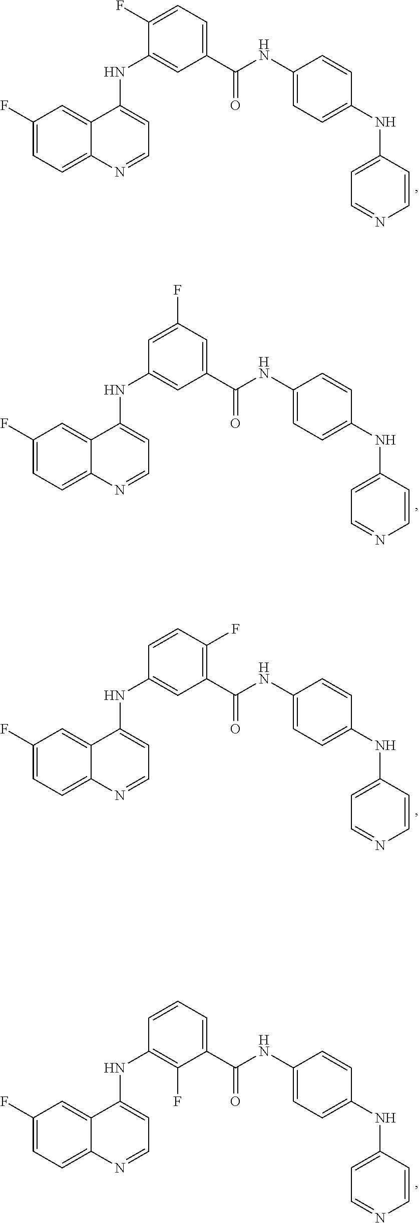 [SMILES: O=C(Nc1ccc(Nc2ccncc2)cc1)c1ccc(F)c(Nc2ccnc3ccc(F)cc23)c1 ; O=C(Nc1ccc(Nc2ccncc2)cc1)c1cc(F)cc(Nc2ccnc3ccc(F)cc23)c1 ; O=C(Nc1ccc(Nc2ccncc2)cc1)c1cc(Nc2ccnc3ccc(F)cc23)ccc1F ; O=C(Nc1ccc(Nc2ccncc2)cc1)c1cccc(Nc2ccnc3ccc(F)cc23)c1F]

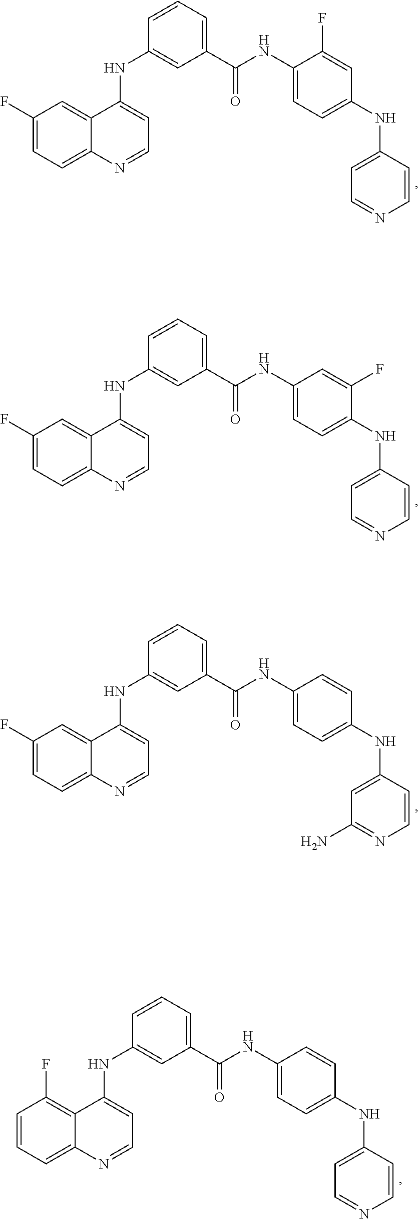 [SMILES: O=C(Nc1ccc(Nc2ccncc2)cc1F)c1cccc(Nc2ccnc3ccc(F)cc23)c1 ; O=C(Nc1ccc(Nc2ccncc2)c(F)c1)c1cccc(Nc2ccnc3ccc(F)cc23)c1 ; Nc1cc(Nc2ccc(NC(=O)c3cccc(Nc4ccnc5ccc(F)cc45)c3)cc2)ccn1 ; O=C(Nc1ccc(Nc2ccncc2)cc1)c1cccc(Nc2ccnc3cccc(F)c23)c1]

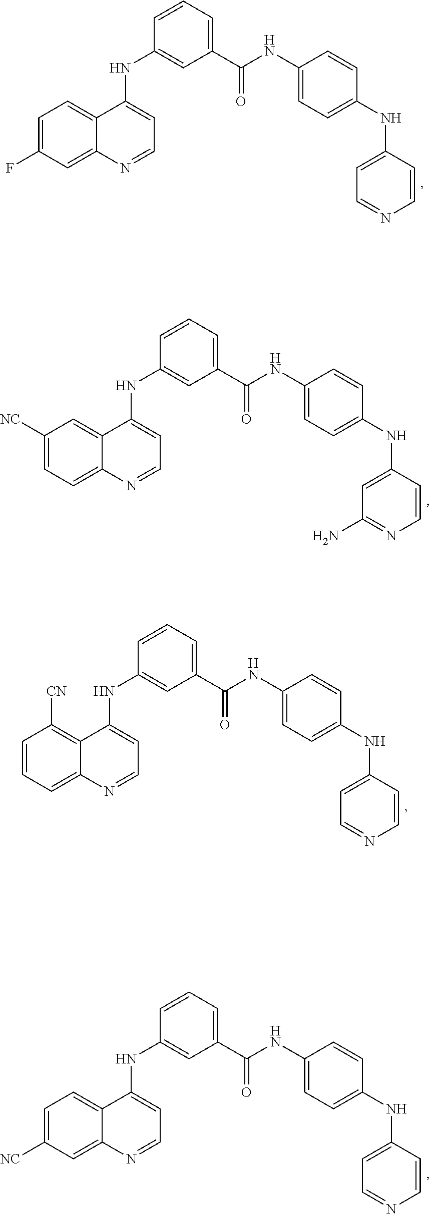 [SMILES: O=C(Nc1ccc(Nc2ccncc2)cc1)c1cccc(Nc2ccnc3cc(F)ccc23)c1 ; N#Cc1ccc2nccc(Nc3cccc(C(=O)Nc4ccc(Nc5ccnc(N)c5)cc4)c3)c2c1 ; N#Cc1cccc2nccc(Nc3cccc(C(=O)Nc4ccc(Nc5ccncc5)cc4)c3)c12 ; N#Cc1ccc2c(Nc3cccc(C(=O)Nc4ccc(Nc5ccncc5)cc4)c3)ccnc2c1]

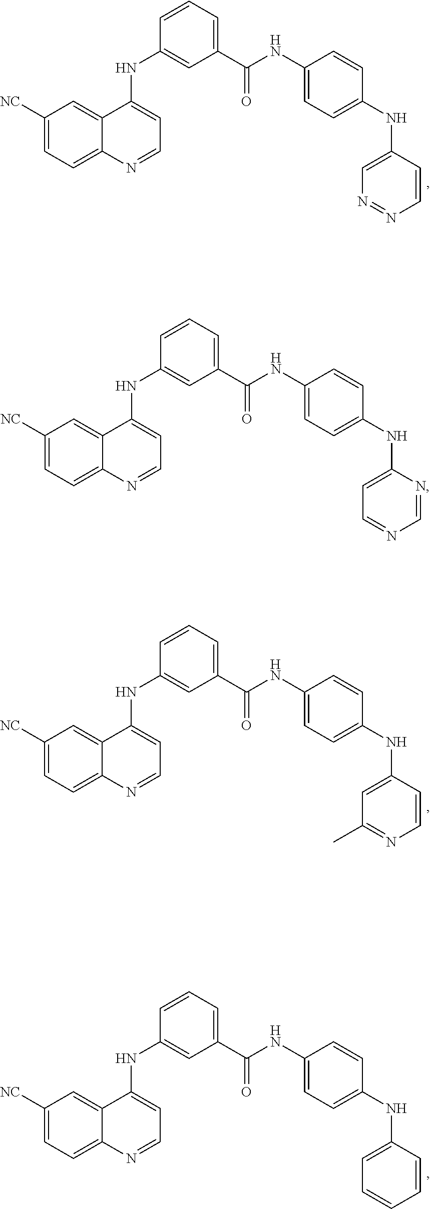 [SMILES: N#Cc1ccc2nccc(Nc3cccc(C(=O)Nc4ccc(Nc5ccnnc5)cc4)c3)c2c1 ; N#Cc1ccc2nccc(Nc3cccc(C(=O)Nc4ccc(Nc5ccncn5)cc4)c3)c2c1 ; Cc1cc(Nc2ccc(NC(=O)c3cccc(Nc4ccnc5ccc(C#N)cc45)c3)cc2)ccn1 ; N#Cc1ccc2nccc(Nc3cccc(C(=O)Nc4ccc(Nc5ccccc5)cc4)c3)c2c1]

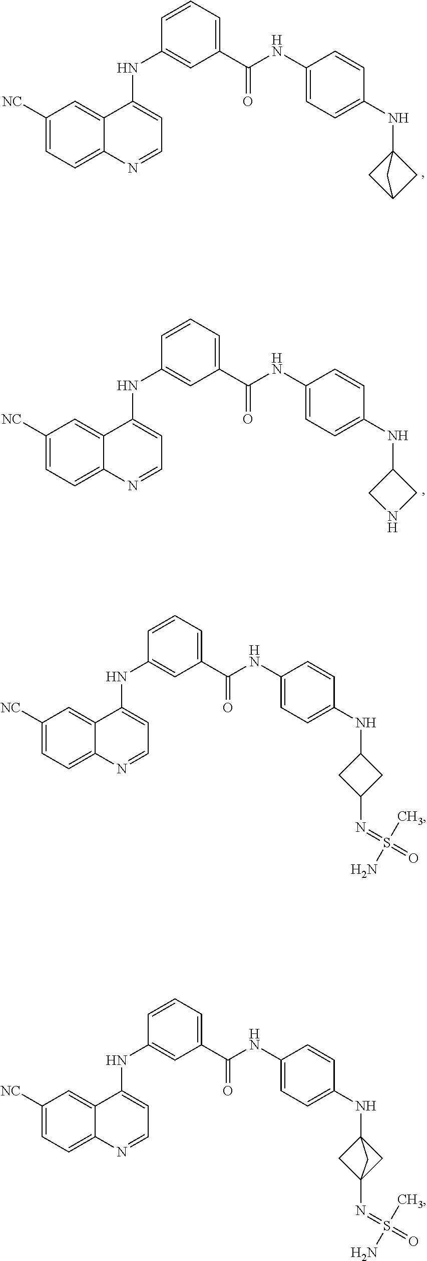 [SMILES: N#Cc1ccc2nccc(Nc3cccc(C(=O)Nc4ccc(NC56CC(C5)C6)cc4)c3)c2c1 ; N#Cc1ccc2nccc(Nc3cccc(C(=O)Nc4ccc(NC5CNC5)cc4)c3)c2c1 ; CS(N)(=O)=NC1CC(Nc2ccc(NC(=O)c3cccc(Nc4ccnc5ccc(C#N)cc45)c3)cc2)C1 ; CS(N)(=O)=NC12CC(Nc3ccc(NC(=O)c4cccc(Nc5ccnc6ccc(C#N)cc56)c4)cc3)(C1)C2]

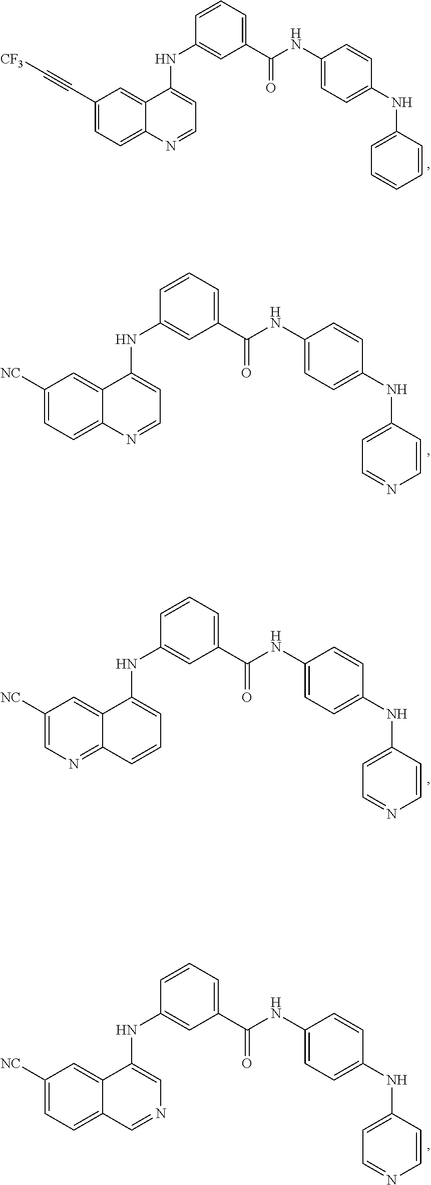 [SMILES: O=C(Nc1ccc(Nc2ccccc2)cc1)c1cccc(Nc2ccnc3ccc(C#CC(F)(F)F)cc23)c1 ; N#Cc1ccc2nccc(Nc3cccc(C(=O)Nc4ccc(Nc5ccncc5)cc4)c3)c2c1 ; N#Cc1cnc2cccc(Nc3cccc(C(=O)Nc4ccc(Nc5ccncc5)cc4)c3)c2c1 ; N#Cc1ccc2cncc(Nc3cccc(C(=O)Nc4ccc(Nc5ccncc5)cc4)c3)c2c1]

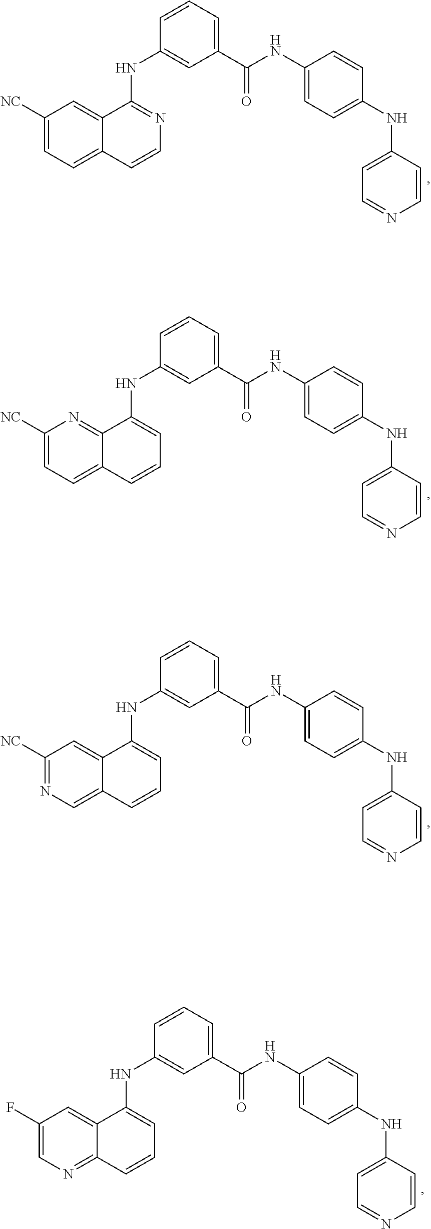 [SMILES: N#Cc1ccc2ccnc(Nc3cccc(C(=O)Nc4ccc(Nc5ccncc5)cc4)c3)c2c1 ; N#Cc1ccc2cccc(Nc3cccc(C(=O)Nc4ccc(Nc5ccncc5)cc4)c3)c2n1 ; N#Cc1cc2c(Nc3cccc(C(=O)Nc4ccc(Nc5ccncc5)cc4)c3)cccc2cn1 ; O=C(Nc1ccc(Nc2ccncc2)cc1)c1cccc(Nc2cccc3ncc(F)cc23)c1]

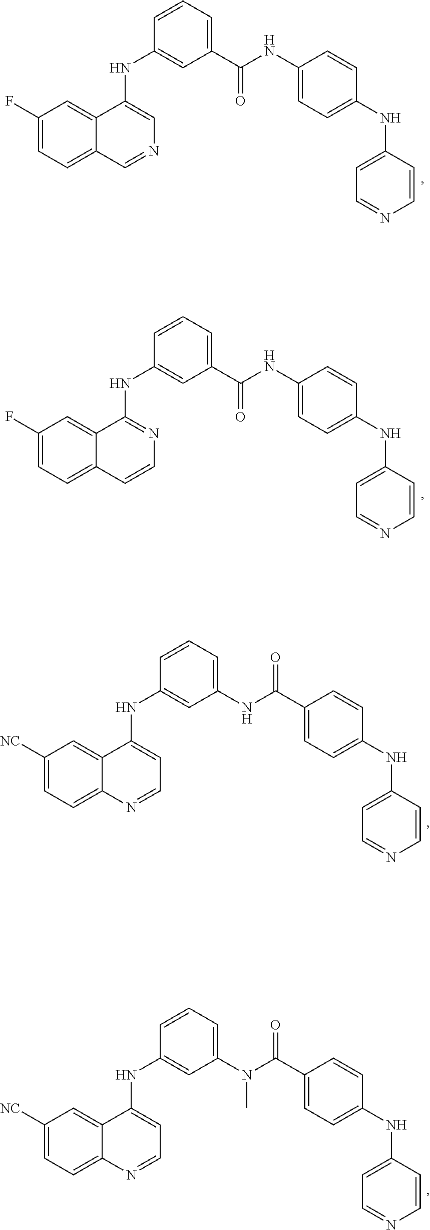 [SMILES: O=C(Nc1ccc(Nc2ccncc2)cc1)c1cccc(Nc2cncc3ccc(F)cc23)c1 ; O=C(Nc1ccc(Nc2ccncc2)cc1)c1cccc(Nc2nccc3ccc(F)cc23)c1 ; N#Cc1ccc2nccc(Nc3cccc(NC(=O)c4ccc(Nc5ccncc5)cc4)c3)c2c1 ; CN(C(=O)c1ccc(Nc2ccncc2)cc1)c1cccc(Nc2ccnc3ccc(C#N)cc23)c1]

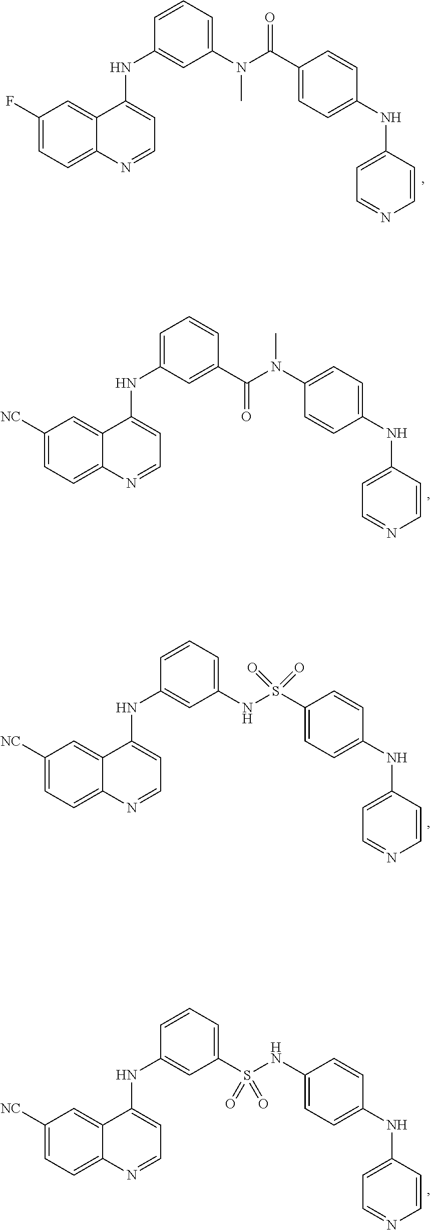 [SMILES: CN(C(=O)c1ccc(Nc2ccncc2)cc1)c1cccc(Nc2ccnc3ccc(F)cc23)c1 ; CN(C(=O)c1cccc(Nc2ccnc3ccc(C#N)cc23)c1)c1ccc(Nc2ccncc2)cc1 ; N#Cc1ccc2nccc(Nc3cccc(NS(=O)(=O)c4ccc(Nc5ccncc5)cc4)c3)c2c1 ; N#Cc1ccc2nccc(Nc3cccc(S(=O)(=O)Nc4ccc(Nc5ccncc5)cc4)c3)c2c1]

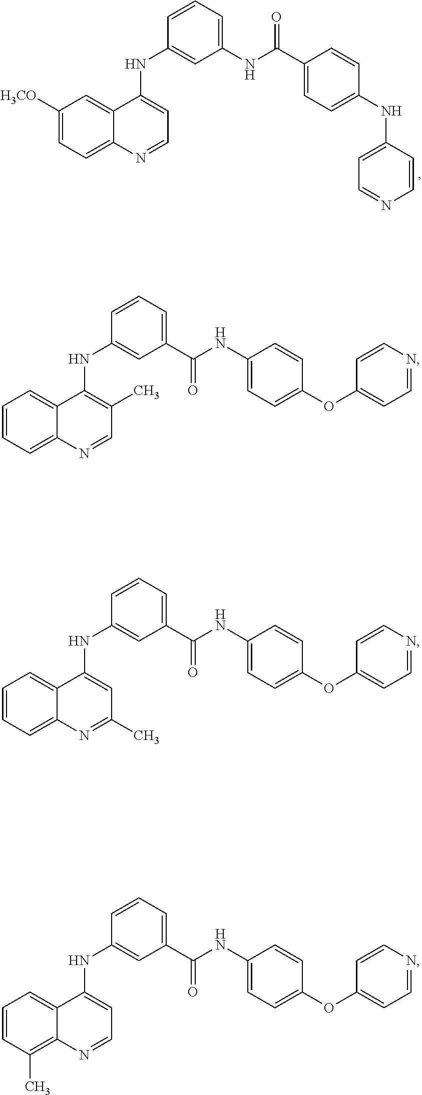 [SMILES: COc1ccc2nccc(Nc3cccc(NC(=O)c4ccc(Nc5ccncc5)cc4)c3)c2c1 ; Cc1cnc2ccccc2c1Nc1cccc(C(=O)Nc2ccc(Oc3ccncc3)cc2)c1 ; Cc1cc(Nc2cccc(C(=O)Nc3ccc(Oc4ccncc4)cc3)c2)c2ccccc2n1 ; Cc1cccc2c(Nc3cccc(C(=O)Nc4ccc(Oc5ccncc5)cc4)c3)ccnc12]

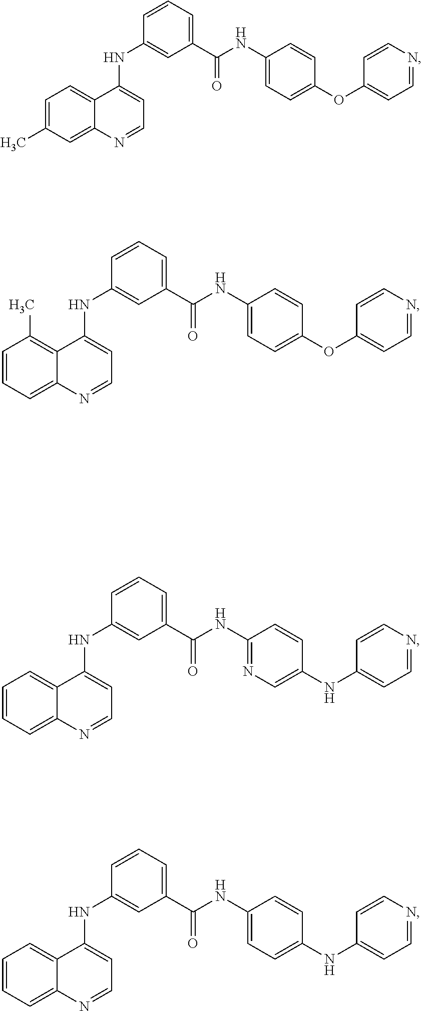 [SMILES: Cc1ccc2c(Nc3cccc(C(=O)Nc4ccc(Oc5ccncc5)cc4)c3)ccnc2c1 ; Cc1cccc2nccc(Nc3cccc(C(=O)Nc4ccc(Oc5ccncc5)cc4)c3)c12 ; O=C(Nc1ccc(Nc2ccncc2)cn1)c1cccc(Nc2ccnc3ccccc23)c1 ; O=C(Nc1ccc(Nc2ccncc2)cc1)c1cccc(Nc2ccnc3ccccc23)c1]

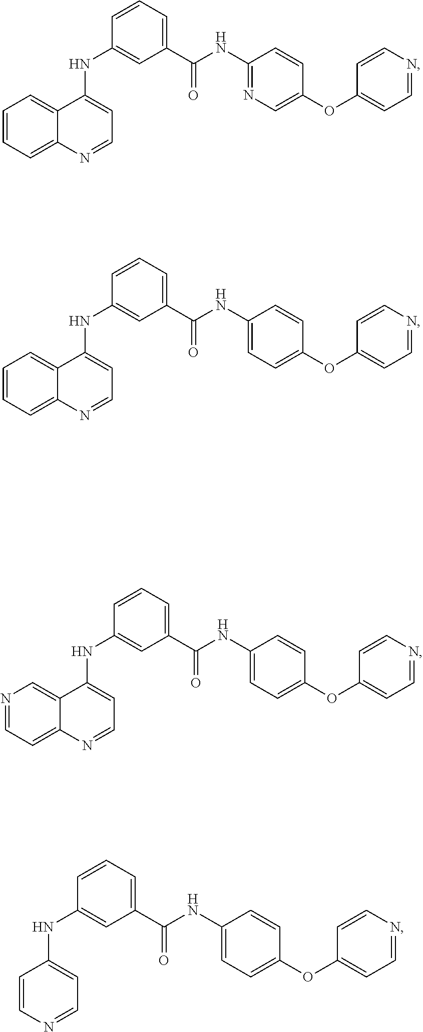 [SMILES: O=C(Nc1ccc(Oc2ccncc2)cn1)c1cccc(Nc2ccnc3ccccc23)c1 ; O=C(Nc1ccc(Oc2ccncc2)cc1)c1cccc(Nc2ccnc3ccccc23)c1 ; O=C(Nc1ccc(Oc2ccncc2)cc1)c1cccc(Nc2ccnc3ccncc23)c1 ; O=C(Nc1ccc(Oc2ccncc2)cc1)c1cccc(Nc2ccncc2)c1]

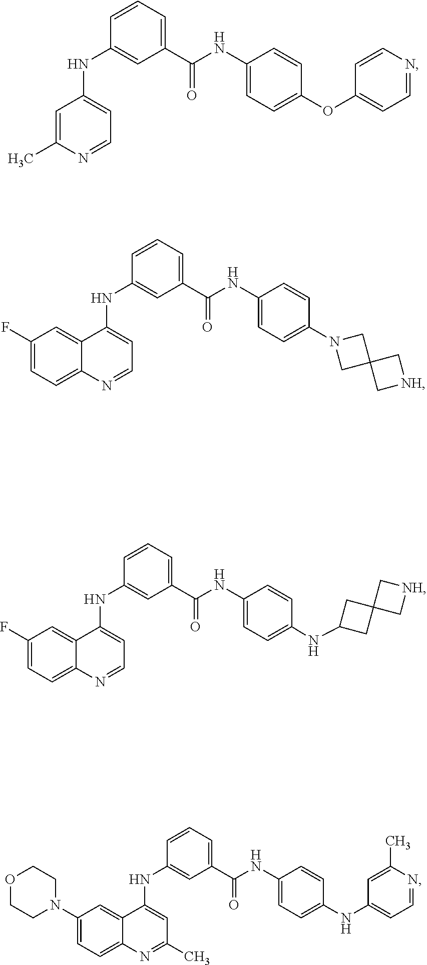 [SMILES: Cc1cc(Nc2cccc(C(=O)Nc3ccc(Oc4ccncc4)cc3)c2)ccn1 ; O=C(Nc1ccc(N2CC3(CNC3)C2)cc1)c1cccc(Nc2ccnc3ccc(F)cc23)c1 ; O=C(Nc1ccc(NC2CC3(CNC3)C2)cc1)c1cccc(Nc2ccnc3ccc(F)cc23)c1 ; Cc1cc(Nc2ccc(NC(=O)c3cccc(Nc4cc(C)nc5ccc(N6CCOCC6)cc45)c3)cc2)ccn1]

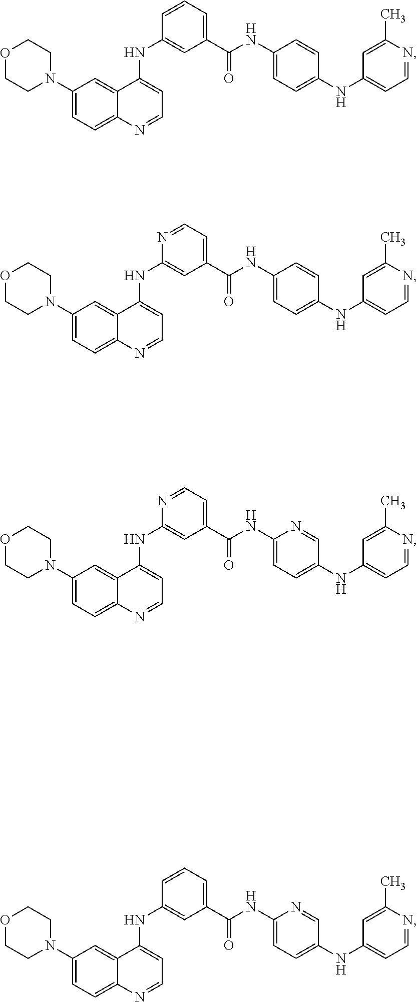 [SMILES: Cc1cc(Nc2ccc(NC(=O)c3cccc(Nc4ccnc5ccc(N6CCOCC6)cc45)c3)cc2)ccn1 ; Cc1cc(Nc2ccc(NC(=O)c3ccnc(Nc4ccnc5ccc(N6CCOCC6)cc45)c3)cc2)ccn1 ; Cc1cc(Nc2ccc(NC(=O)c3ccnc(Nc4ccnc5ccc(N6CCOCC6)cc45)c3)nc2)ccn1 ; Cc1cc(Nc2ccc(NC(=O)c3cccc(Nc4ccnc5ccc(N6CCOCC6)cc45)c3)nc2)ccn1]

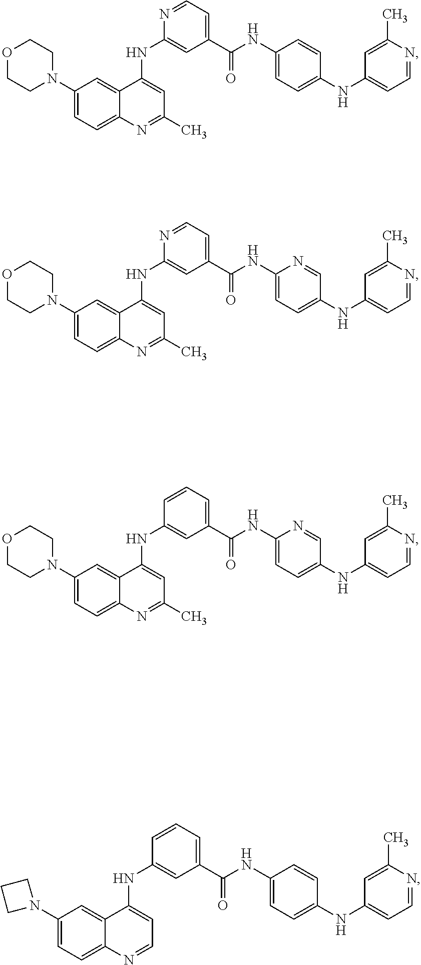 [SMILES: Cc1cc(Nc2ccc(NC(=O)c3ccnc(Nc4cc(C)nc5ccc(N6CCOCC6)cc45)c3)cc2)ccn1 ; Cc1cc(Nc2ccc(NC(=O)c3ccnc(Nc4cc(C)nc5ccc(N6CCOCC6)cc45)c3)nc2)ccn1 ; Cc1cc(Nc2ccc(NC(=O)c3cccc(Nc4cc(C)nc5ccc(N6CCOCC6)cc45)c3)nc2)ccn1 ; Cc1cc(Nc2ccc(NC(=O)c3cccc(Nc4ccnc5ccc(N6CCC6)cc45)c3)cc2)ccn1]

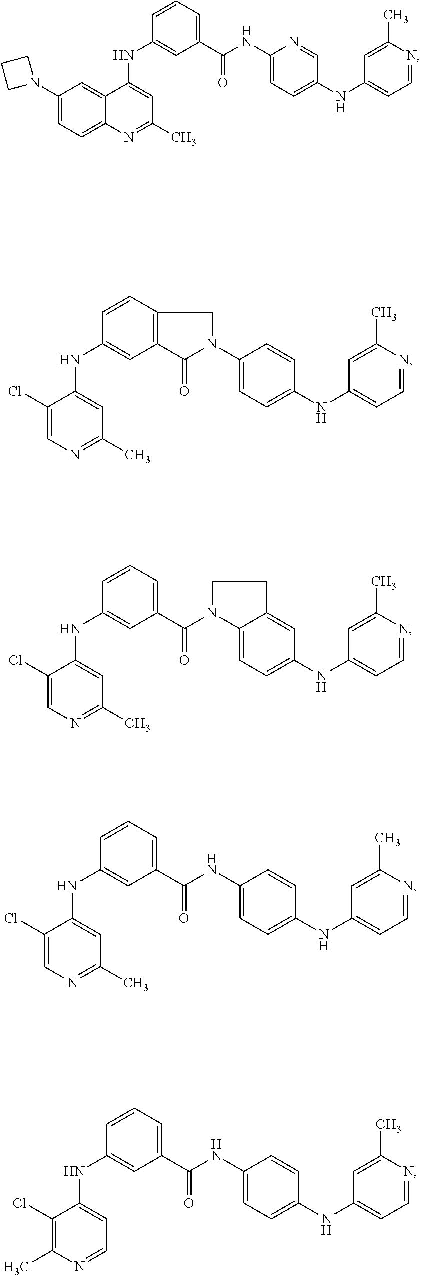 [SMILES: Cc1cc(Nc2ccc(NC(=O)c3cccc(Nc4cc(C)nc5ccc(N6CCC6)cc45)c3)nc2)ccn1 ; Cc1cc(Nc2ccc(N3Cc4ccc(Nc5cc(C)ncc5Cl)cc4C3=O)cc2)ccn1 ; Cc1cc(Nc2ccc3c(c2)CCN3C(=O)c2cccc(Nc3cc(C)ncc3Cl)c2)ccn1 ; Cc1cc(Nc2ccc(NC(=O)c3cccc(Nc4cc(C)ncc4Cl)c3)cc2)ccn1 ; Cc1cc(Nc2ccc(NC(=O)c3cccc(Nc4ccnc(C)c4Cl)c3)cc2)ccn1]

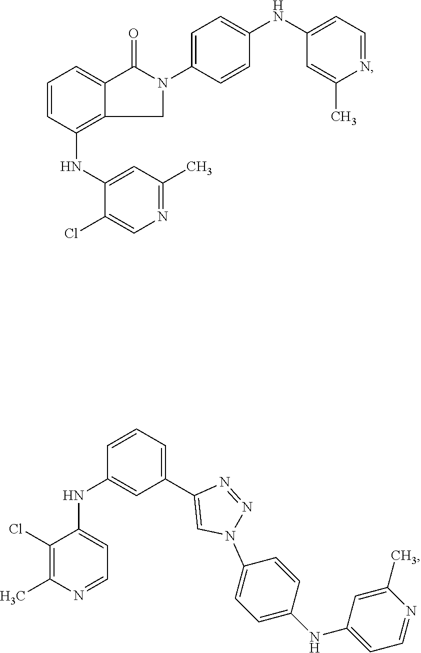 [SMILES: Cc1cc(Nc2ccc(N3Cc4c(Nc5cc(C)ncc5Cl)cccc4C3=O)cc2)ccn1 ; Cc1cc(Nc2ccc(-n3cc(-c4cccc(Nc5ccnc(C)c5Cl)c4)nn3)cc2)ccn1]

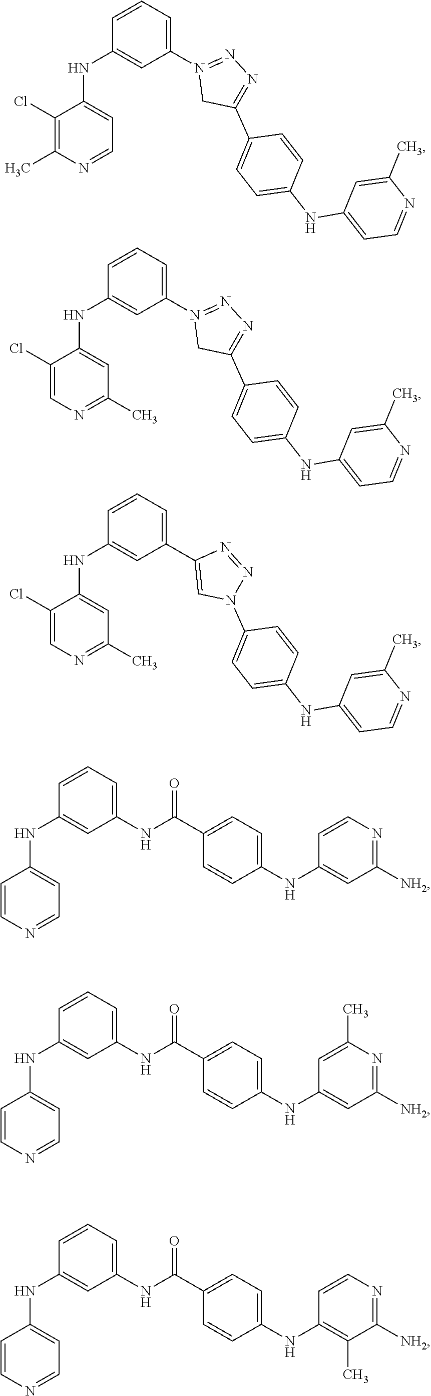

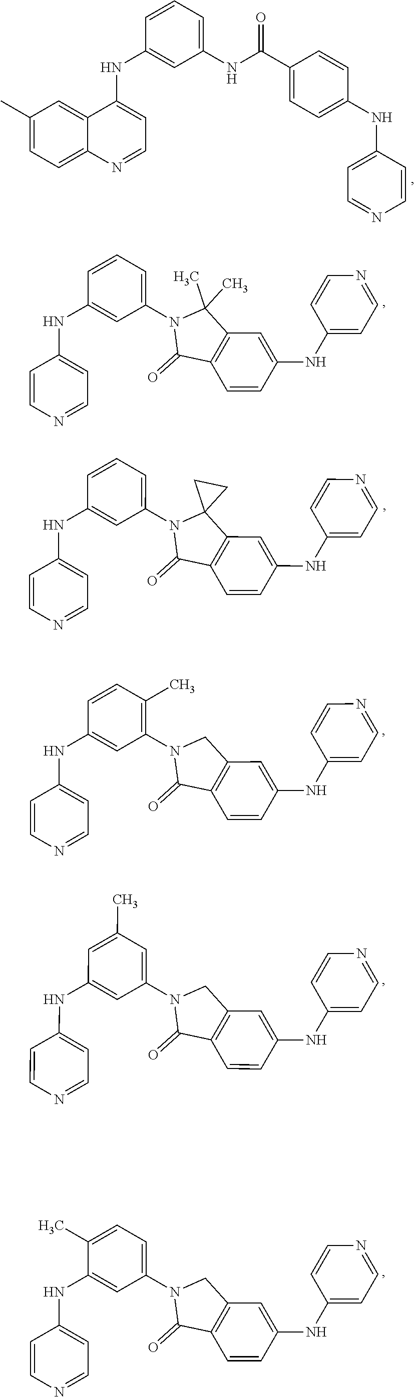 [SMILES: Cc1ccc2nccc(Nc3cccc(NC(=O)c4ccc(Nc5ccncc5)cc4)c3)c2c1 ; CC1(C)c2cc(Nc3ccncc3)ccc2C(=O)N1c1cccc(Nc2ccncc2)c1 ; O=C1c2ccc(Nc3ccncc3)cc2C2(CC2)N1c1cccc(Nc2ccncc2)c1 ; Cc1ccc(Nc2ccncc2)cc1N1Cc2cc(Nc3ccncc3)ccc2C1=O ; Cc1cc(Nc2ccncc2)cc(N2Cc3cc(Nc4ccncc4)ccc3C2=O)c1 ; Cc1ccc(N2Cc3cc(Nc4ccncc4)ccc3C2=O)cc1Nc1ccncc1]

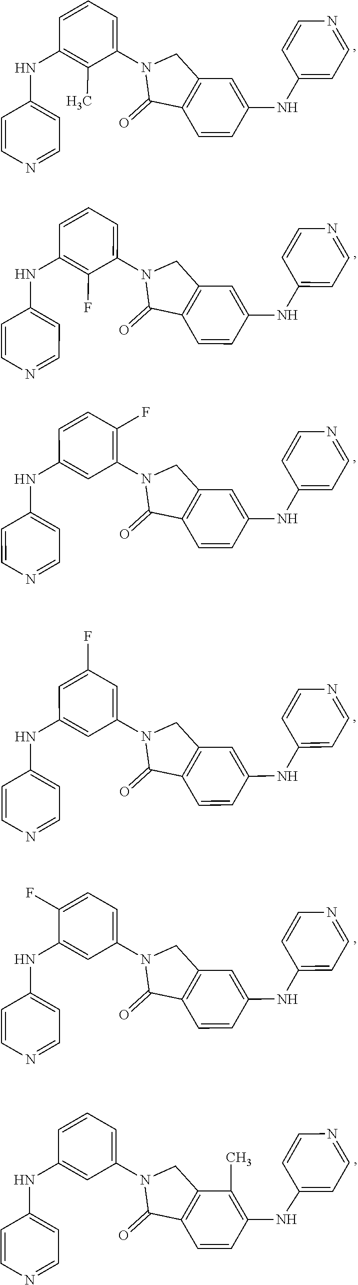 [SMILES: Cc1c(Nc2ccncc2)cccc1N1Cc2cc(Nc3ccncc3)ccc2C1=O ; O=C1c2ccc(Nc3ccncc3)cc2CN1c1cccc(Nc2ccncc2)c1F ; O=C1c2ccc(Nc3ccncc3)cc2CN1c1cc(Nc2ccncc2)ccc1F ; O=C1c2ccc(Nc3ccncc3)cc2CN1c1cc(F)cc(Nc2ccncc2)c1 ; O=C1c2ccc(Nc3ccncc3)cc2CN1c1ccc(F)c(Nc2ccncc2)c1 ; Cc1c(Nc2ccncc2)ccc2c1CN(c1cccc(Nc3ccncc3)c1)C2=O]

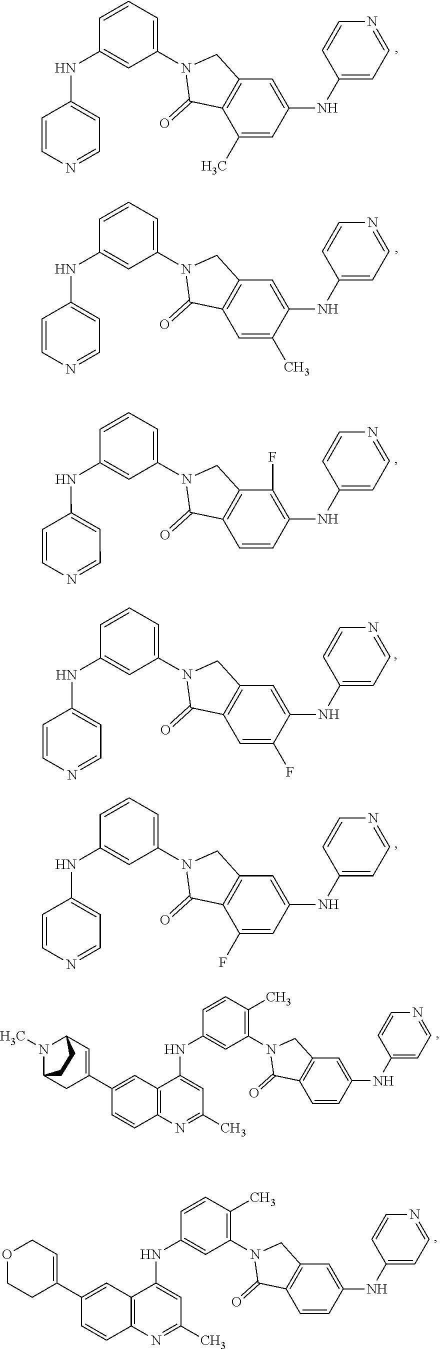

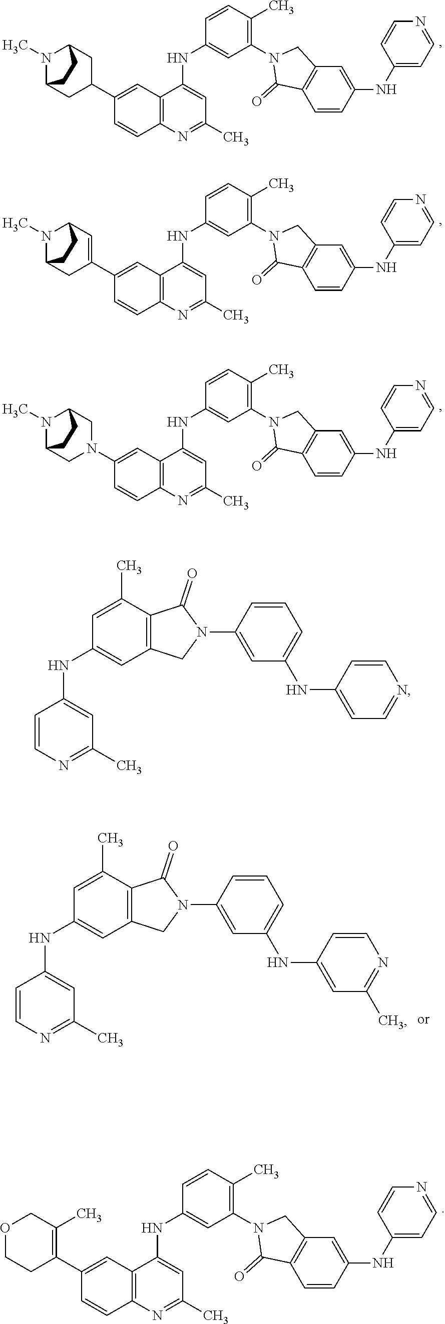

In any one of the embodiments disclosed herein, the compound of Formula Ib is

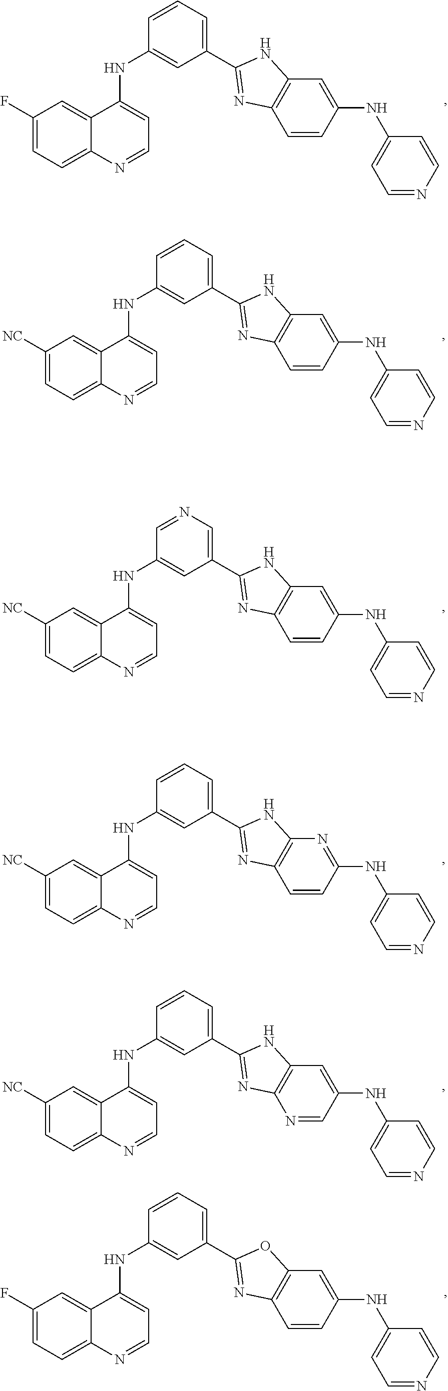 [SMILES: Fc1ccc2nccc(Nc3cccc(-c4nc5ccc(Nc6ccncc6)cc5[nH]4)c3)c2c1 ; N#Cc1ccc2nccc(Nc3cccc(-c4nc5ccc(Nc6ccncc6)cc5[nH]4)c3)c2c1 ; N#Cc1ccc2nccc(Nc3cncc(-c4nc5ccc(Nc6ccncc6)cc5[nH]4)c3)c2c1 ; N#Cc1ccc2nccc(Nc3cccc(-c4nc5ccc(Nc6ccncc6)nc5[nH]4)c3)c2c1 ; N#Cc1ccc2nccc(Nc3cccc(-c4nc5ncc(Nc6ccncc6)cc5[nH]4)c3)c2c1 ; Fc1ccc2nccc(Nc3cccc(-c4nc5ccc(Nc6ccncc6)cc5o4)c3)c2c1]

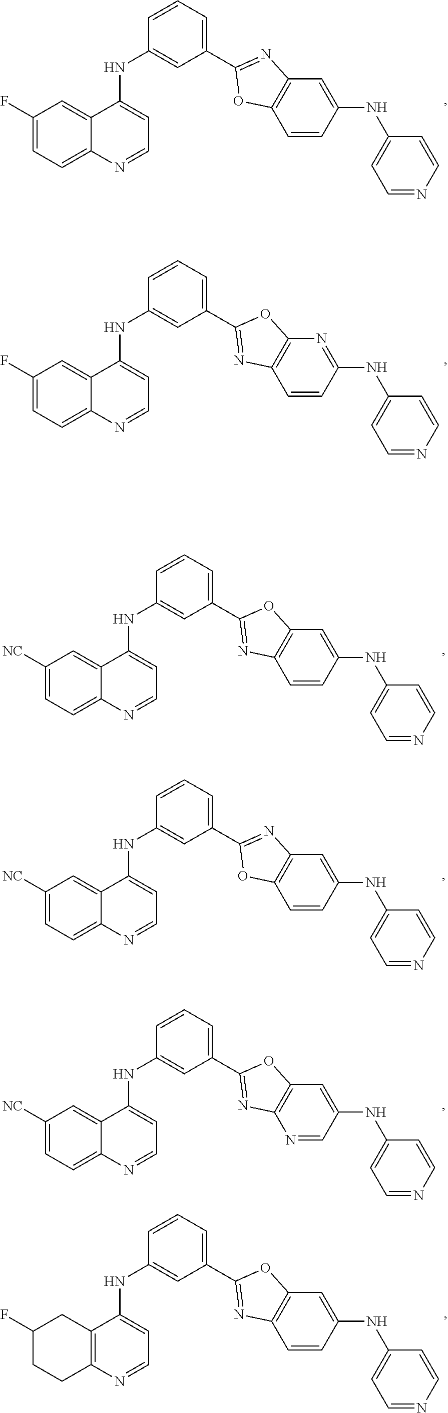 [SMILES: Fc1ccc2nccc(Nc3cccc(-c4nc5cc(Nc6ccncc6)ccc5o4)c3)c2c1 ; Fc1ccc2nccc(Nc3cccc(-c4nc5ccc(Nc6ccncc6)nc5o4)c3)c2c1 ; N#Cc1ccc2nccc(Nc3cccc(-c4nc5ccc(Nc6ccncc6)cc5o4)c3)c2c1 ; N#Cc1ccc2nccc(Nc3cccc(-c4nc5cc(Nc6ccncc6)ccc5o4)c3)c2c1 ; N#Cc1ccc2nccc(Nc3cccc(-c4nc5ncc(Nc6ccncc6)cc5o4)c3)c2c1 ; FC1CCc2nccc(Nc3cccc(-c4nc5ccc(Nc6ccncc6)cc5o4)c3)c2C1]

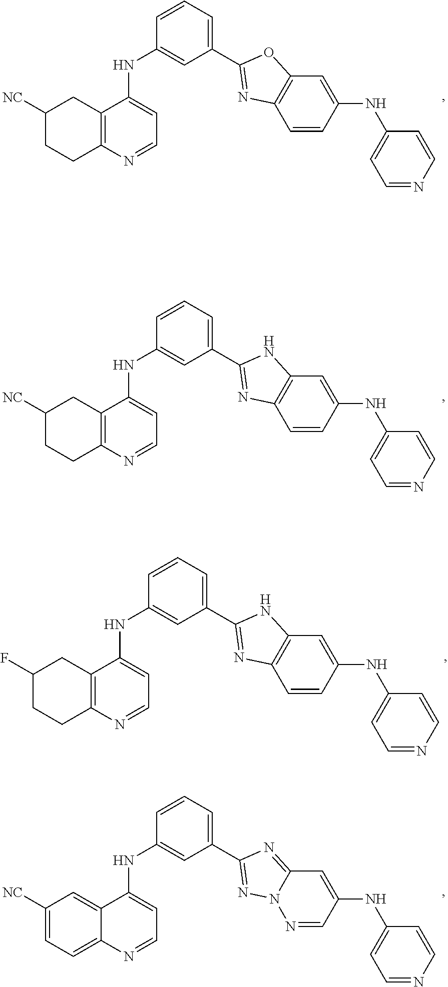 [SMILES: N#CC1CCc2nccc(Nc3cccc(-c4nc5ccc(Nc6ccncc6)cc5o4)c3)c2C1 ; N#CC1CCc2nccc(Nc3cccc(-c4nc5ccc(Nc6ccncc6)cc5[nH]4)c3)c2C1 ; FC1CCc2nccc(Nc3cccc(-c4nc5ccc(Nc6ccncc6)cc5[nH]4)c3)c2C1 ; N#Cc1ccc2nccc(Nc3cccc(-c4nc5cc(Nc6ccncc6)cnn5n4)c3)c2c1]

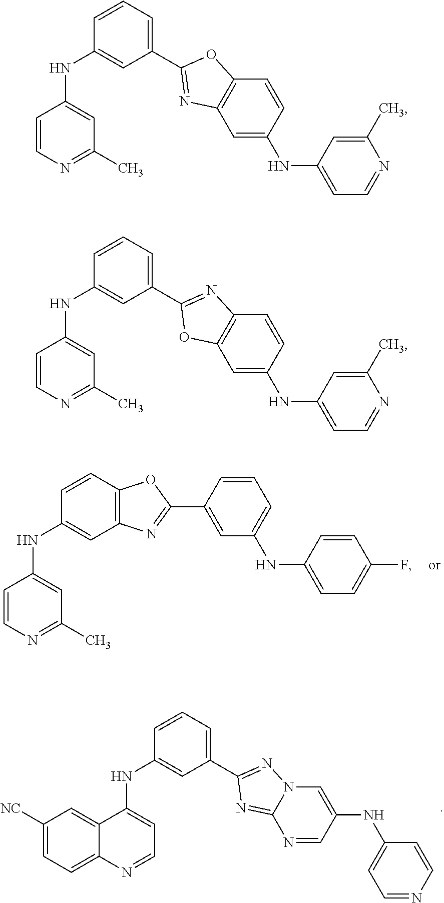 [SMILES: Cc1cc(Nc2cccc(-c3nc4cc(Nc5ccnc(C)c5)ccc4o3)c2)ccn1 ; Cc1cc(Nc2cccc(-c3nc4ccc(Nc5ccnc(C)c5)cc4o3)c2)ccn1 ; Cc1cc(Nc2ccc3oc(-c4cccc(Nc5ccc(F)cc5)c4)nc3c2)ccn1 ; N#Cc1ccc2nccc(Nc3cccc(-c4nc5ncc(Nc6ccncc6)cn5n4)c3)c2c1]

In any one of the embodiments disclosed herein, the compound of Formula Ic is

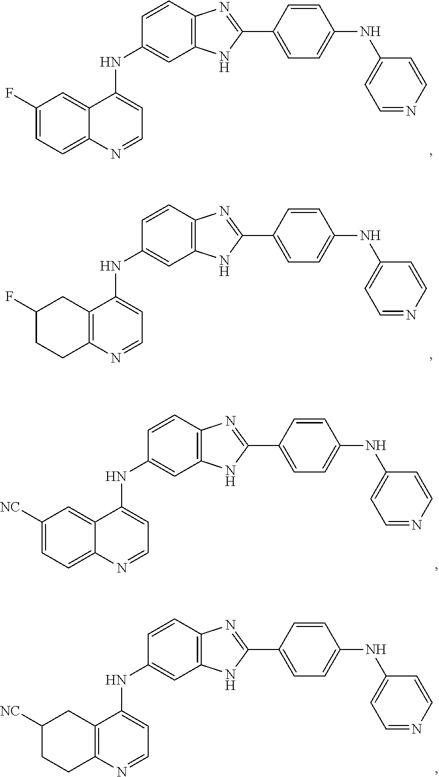 [SMILES: Fc1ccc2nccc(Nc3ccc4nc(-c5ccc(Nc6ccncc6)cc5)[nH]c4c3)c2c1 ; FC1CCc2nccc(Nc3ccc4nc(-c5ccc(Nc6ccncc6)cc5)[nH]c4c3)c2C1 ; N#Cc1ccc2nccc(Nc3ccc4nc(-c5ccc(Nc6ccncc6)cc5)[nH]c4c3)c2c1 ; N#CC1CCc2nccc(Nc3ccc4nc(-c5ccc(Nc6ccncc6)cc5)[nH]c4c3)c2C1]

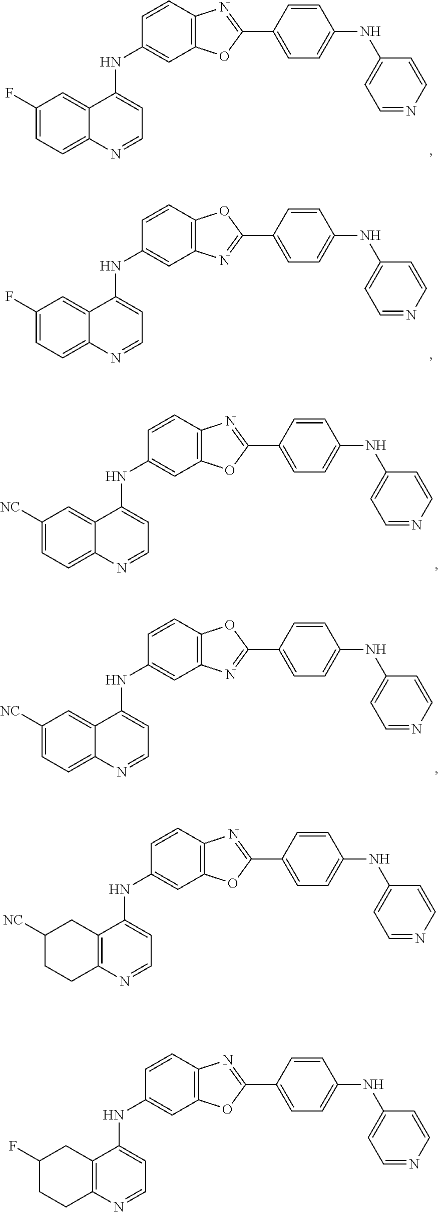 [SMILES: Fc1ccc2nccc(Nc3ccc4nc(-c5ccc(Nc6ccncc6)cc5)oc4c3)c2c1 ; Fc1ccc2nccc(Nc3ccc4oc(-c5ccc(Nc6ccncc6)cc5)nc4c3)c2c1 ; N#Cc1ccc2nccc(Nc3ccc4nc(-c5ccc(Nc6ccncc6)cc5)oc4c3)c2c1 ; N#Cc1ccc2nccc(Nc3ccc4oc(-c5ccc(Nc6ccncc6)cc5)nc4c3)c2c1 ; N#CC1CCc2nccc(Nc3ccc4nc(-c5ccc(Nc6ccncc6)cc5)oc4c3)c2C1 ; FC1CCc2nccc(Nc3ccc4nc(-c5ccc(Nc6ccncc6)cc5)oc4c3)c2C1]

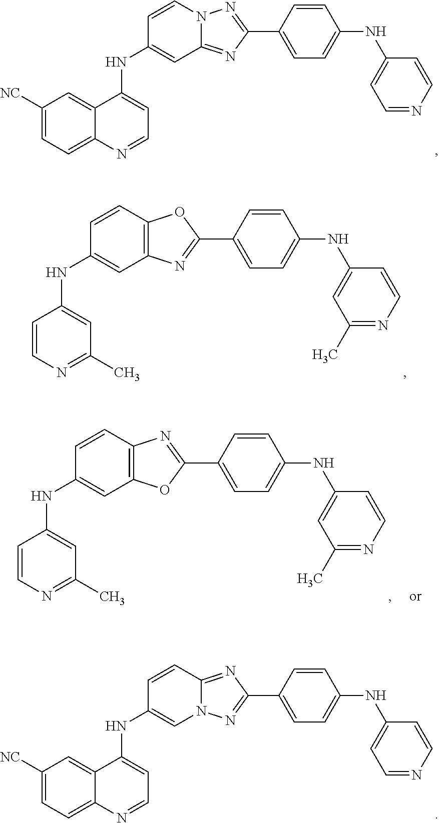 [SMILES: N#Cc1ccc2nccc(Nc3ccn4nc(-c5ccc(Nc6ccncc6)cc5)nc4c3)c2c1 ; Cc1cc(Nc2ccc(-c3nc4cc(Nc5ccnc(C)c5)ccc4o3)cc2)ccn1 ; Cc1cc(Nc2ccc(-c3nc4ccc(Nc5ccnc(C)c5)cc4o3)cc2)ccn1 ; N#Cc1ccc2nccc(Nc3ccc4nc(-c5ccc(Nc6ccncc6)cc5)nn4c3)c2c1]

In any one of the embodiments disclosed herein, the compound is

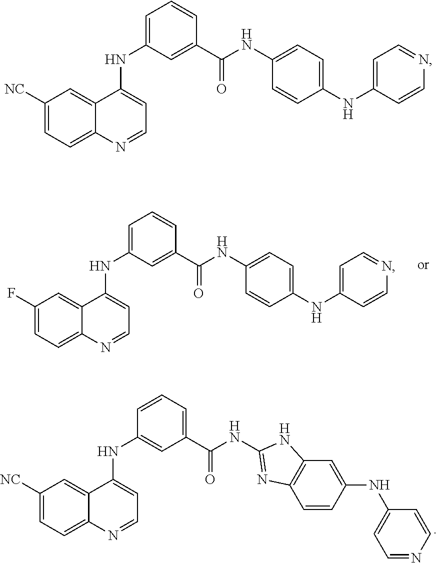 [SMILES: N#Cc1ccc2nccc(Nc3cccc(C(=O)Nc4ccc(Nc5ccncc5)cc4)c3)c2c1 ; O=C(Nc1ccc(Nc2ccncc2)cc1)c1cccc(Nc2ccnc3ccc(F)cc23)c1 ; "or" ; N#Cc1ccc2nccc(Nc3cccc(C(=O)Nc4nc5ccc(Nc6ccncc6)cc5[nH]4)c3)c2c1]

In any one of the embodiments disclosed herein, the compound is

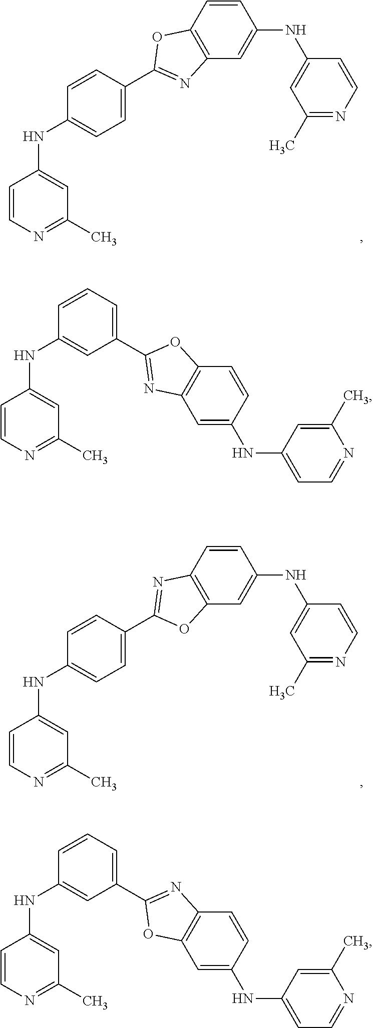 [SMILES: Cc1cc(Nc2ccc(-c3nc4cc(Nc5ccnc(C)c5)ccc4o3)cc2)ccn1 ; Cc1cc(Nc2cccc(-c3nc4cc(Nc5ccnc(C)c5)ccc4o3)c2)ccn1 ; Cc1cc(Nc2ccc(-c3nc4ccc(Nc5ccnc(C)c5)cc4o3)cc2)ccn1 ; Cc1cc(Nc2cccc(-c3nc4ccc(Nc5ccnc(C)c5)cc4o3)c2)ccn1]

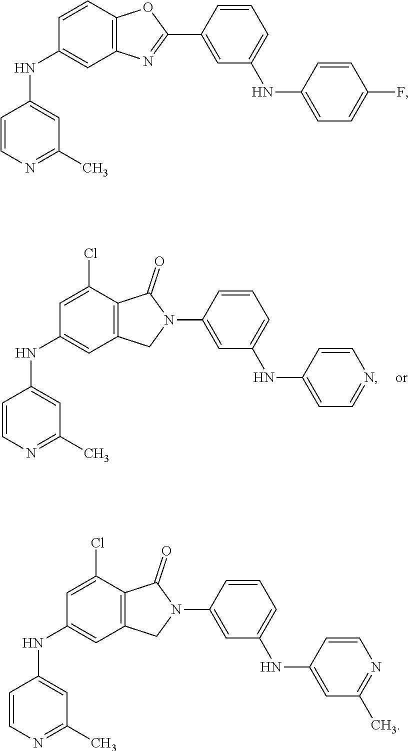 [SMILES: Cc1cc(Nc2ccc3oc(-c4cccc(Nc5ccc(F)cc5)c4)nc3c2)ccn1 ; Cc1cc(Nc2cc(Cl)c3c(c2)CN(c2cccc(Nc4ccncc4)c2)C3=O)ccn1 ; Cc1cc(Nc2cccc(N3Cc4cc(Nc5ccnc(C)c5)cc(Cl)c4C3=O)c2)ccn1]

In any one of the embodiments disclosed herein, the compound is selected from the group consisting of compounds 2-45 in Examples 2-45, respectively.

In another aspect, a method of treating a disease in a subject in need thereof is described, including administering to the subject an effective amount of the compound of any one of the embodiments disclosed herein.

In any one of the embodiments described herein, the disease is selected from the group consisting of neurodegenerative disease, cachexia, anorexia, obesity, obesity's complication, inflammatory disease, viral-induced inflammatory reaction, Gulf War Syndrome, tuberous sclerosis, retinitis pigmentosa, transplant rejection, cancer, an autoimmune disease, ischemic tissue injury, traumatic tissue injury and a combination thereof.

In any one of the embodiments described herein, the disease is neurodegenerative disease.

In any one of the embodiments described herein, the neurodegenerative disease is selected from the group consisting of Parkinson's disease, Alzheimer's disease, amyotrophic lateral sclerosis, Motor Neuron Disease, Huntington's disease, HIV-induced neurodegeneration, Lewy Body Disease, spinal muscular atrophy, prion disease, spinocerebellar ataxia, familial amyloid polyneuropathy, multiple sclerosis, and a combination thereof.

In any one of the embodiments described herein, the disease is cachexia or anorexia.

In any one of the embodiments described herein, the disease is obesity or obesity's complication.

In any one of the embodiments described herein, the obesity's complication is selected from the group consisting of glucose intolerance, hepatic steatosis, dyslipidemia, and a combination thereof.

In any one of the embodiments described herein, the disease is inflammatory disease.

In any one of the embodiments described herein, the inflammatory disease is selected from the group consisting of atopic dermatitis, allergy, asthma, and a combination thereof.

In any one of the embodiments described herein, the disease is viral-induced inflammatory reaction.

In any one of the embodiments described herein, the viral-induced inflammatory reaction is SARS-induced inflammatory pneumonitis, coronavirus disease 2019, or a combination thereof.

In any one of the embodiments described herein, the disease is Gulf War Syndrome or tuberous sclerosis.

In any one of the embodiments described herein, the disease is retinitis pigmentosa or transplant rejection.

In any one of the embodiments described herein, the disease is ischemic tissue injury or traumatic tissue injury.

In any one of the embodiments described herein, the disease is cancer.

In any one of the embodiments described herein, the cancer is selected from the group consisting of adult T-cell leukemia/lymphoma, bladder, brain, breast, cervical, colorectal, esophageal, kidney, liver, lung, nasopharyngeal, pancreatic, prostate, skin, stomach, uterine, ovarian, and testicular cancer.

In any one of the embodiments described herein, the cancer is leukemia.

In any one of the embodiments described herein, the leukemia is adult T-cell leukemia/lymphoma.

In any one of the embodiments described herein, the adult T-cell leukemia/lymphoma is caused by human T-cell lymphotropic virus.

In any one of the embodiments described herein, the disease is autoimmune disease.

In any one of the embodiments described herein, the autoimmune disease is selected from the group consisting of achalasia, Addison's disease, adult Still's disease, agammaglobulinemia, alopecia areata, amyloidosis, ankylosing spondylitis, anti-glomerular basement membrane disease, anti-tubular basement membrane antibody nephritis, antiphospholipid syndrome, autoimmune angioedema, autoimmune dysautonomia, autoimmune encephalomyelitis, autoimmune hepatitis, autoimmune inner ear disease, autoimmune myocarditis, autoimmune oophoritis, autoimmune orchitis, autoimmune pancreatitis, autoimmune retinopathy, autoimmune urticaria, axonal and neuronal neuropathy, Baló disease, Behcet's disease, benign mucosal pemphigoid, bullous pemphigoid, Castleman disease, celiac disease, Chagas disease, chronic inflammatory demyelinating polyneuropathy, chronic recurrent multifocal osteomyelitis, Churg-Strauss syndrome, eosinophilic granulomatosis, cicatricial pemphigoid, Cogan's syndrome, cold agglutinin disease, congenital heart block, Coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura, pemphigoid gestationis, hidradenitis suppurativa (acne inversa), hypogammalglobulinemia, IgA nephropathy, IgG4-related sclerosing disease, immune thrombocytopenia purpura, inclusion body myositis, interstitial cystitis, juvenile arthritis, juvenile diabetes (type 1 diabetes), juvenile myositis, Kawasaki disease, Lambert-Eaton syndrome, leukocytoclastic vasculitis, lichen planus, lichen sclerosus, ligneous conjunctivitis, linear IgA disease, lupus, chronic Lyme disease, Meniere's disease, microscopic polyangiitis, mixed connective tissue disease, Mooren's ulcer, Mucha-Habermann disease, multifocal motor neuropathy, multiple sclerosis, myasthenia gravis, myositis, narcolepsy, neonatal lupus, neuromyelitis optica, neutropenia, ocular cicatricial pemphigoid, optic neuritis, palindromic rheumatism, pediatric autoimmune neuropsychiatric disorder, paraneoplastic cerebellar degeneration, paroxysmal nocturnal hemoglobinuria, Parry Romberg syndrome, pars planitis (peripheral uveitis), Parsonage-Turner syndrome, pemphigus, peripheral neuropathy, perivenous encephalomyelitis, pernicious anemia, POEMS syndrome, polyarteritis nodosa, polyglandular syndrome type I, polyglandular syndrome type II, polyglandular syndrome type III, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia, pyoderma gangrenosum, Raynaud's phenomenon, reactive arthritis, reflex sympathetic dystrophy, relapsing polychondritis, restless legs syndrome, retroperitoneal fibrosis, rheumatic fever, rheumatoid arthritis, sarcoidosis, Schmidt syndrome, scleritis, scleroderma, Sjögren's syndrome, sperm and testicular autoimmunity, stiff person syndrome, subacute bacterial endocarditis, Susac's syndrome, sympathetic ophthalmia, Takayasu's arteritis, temporal arteritis (giant cell arteritis), thrombocytopenia purpura, Tolosa-Hunt syndrome, transverse myelitis, ulcerative colitis, undifferentiated connective tissue disease, uveitis, vasculitis, vitiligo, Vogt-Koyanagi-Harada disease, and a combination thereof.

In any one of the embodiments described herein, the compound modulates Akt3 in immune cells.

In any one of the embodiments described herein, the immune cells are selected from the group consisting of T cells, B cells, macrophages, and glial cells.

In any one of the embodiments described herein, the glial cells are astrocytes, microglia, or oligodendrocytes.

In any one of the embodiments described herein, the T cells are T regulatory cells.

In any one of the embodiments described herein, the compound activates Akt3 signaling.

In any one of the embodiments described herein, the compound inhibits Akt3 signaling.

In any one of the embodiments described herein, the compound increases T regulatory cell activity or production.

In any one of the embodiments described herein, the compound decreases T regulatory cell activity or production.

In any one of the embodiments described herein, the method further includes administering a second therapeutic agent to the subject.

In any one of the embodiments described herein, the second therapeutic agent is selected from the group consisting of a nutrient supplementation, a chemotherapeutic, an anti-inflammatory, an immunosuppressant, a cholinesterase inhibitor, an antidepressant, an anxiolytic, an antipsychotic, riluzole, edavarone, a dopamine agonist, a MAO B inhibitor, a catechol O-methyltransferase inhibitor, an anticholinergic, an anticonvulsant, tetrabenazine, carbidopa-levodopa, an antispastic, an antibody, a fusion protein, an enzyme, a nucleic acid, a ribonucleic acid, an anti-proliferative, a cytotoxic agent, an appetite stimulant, a 5-HT3 antagonist, a Cox-2 inhibitor, and a combination thereof.

In any one of the embodiments described herein, the method further includes treating the subject with an immune therapeutic agent, an immune modulator, an costimulatory activating agonist, a cytokine, a chemokine, a chemokine factor, an oncolytic virus, a biologics, a vaccine, a small molecule, a targeted therapy, an anti-inflammatory agent, a cell therapy, a chemotherapeutic agent, or radiation therapy.

Any one of the embodiments disclosed herein may be properly combined with any other embodiment disclosed herein. The combination of any one of the embodiments disclosed herein with any other embodiments disclosed herein is expressly contemplated. Specifically, the selection of one or more embodiments for one substituent group can be properly combined with the selection of one or more particular embodiments for any other substituent group. Such combination can be made in any one or more embodiments of the application described herein or any formula described herein.

### DESCRIPTION OF THE DRAWINGS

The application is described with reference to the following figures, which are presented for the purpose of illustration only and are not intended to be limiting. In the Drawings:

FIG. 1 shows evaluation of iTreg induction (FoxP3) from human CD4 T cells treated with Compound 22 in the presence of anti-CD3/anti-CD28/IL-2/TGFβ, according to one or more embodiments described herein.

FIG. 2 shows evaluation of iTreg induction (FoxP3) from human CD4 T cells treated with Compounds 31 and 39-43, according to one or more embodiments described herein.

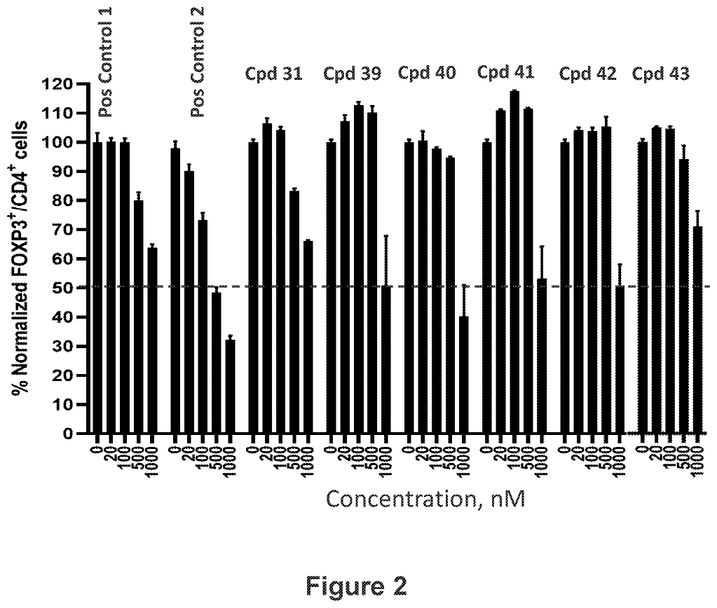

FIG. 3 shows evaluation of iTreg induction from human CD4 T cells treated with Compounds 44-45, according to one or more embodiments described herein.

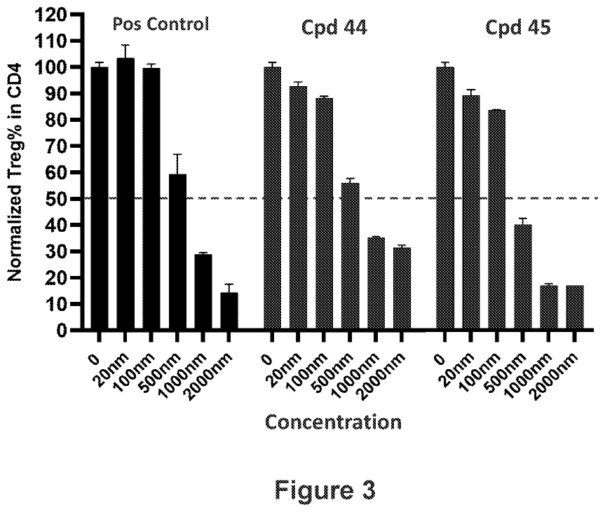

### DETAILED DESCRIPTION OF THE INVENTION

### Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any compositions, methods, and materials similar or equivalent to those described herein can be used in the practice or testing of the present invention.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approximately ±10%. In some embodiments, the values may be either above or below the stated value in a range of approximately ±5%. In some embodiments, the values may be either above or below the stated value in a range of approximately ±2%. In other embodiments, the values may be either above or below the stated value in a range of approximately ±1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “exemplary”, “such as”, “for example”, “including, but not limited to”) provided herein, is intended merely to better illuminate the invention and does not pose a limitation on the scope of the invention unless otherwise indicated.

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The terms “alkyl” and “alk” refer to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “(C₁-C₄)alkyl” refers to a straight or branched chain alkane (hydrocarbon) radical containing from 1 to 4 carbon atoms, such as methyl, ethyl, propyl, isopropyl, n-butyl, t-butyl, and isobutyl. “Substituted alkyl” refers to an alkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, bicycloalkyl, spiroalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, —N═S(═O)(Rₐ), S(═O)(═NRₐ)(═N(Rₐ)₂) (linked to the molecule via S or N), P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NRₐS(═O)₂NR_{b}R_{c}, NRₐP(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and Rₐ is independently hydrogen, alkyl, cycloalkyl, heterocycle, aryl, or said R_{b} and R_{c} together with the N to which they are bonded optionally form a heterocycle, and each occurrence of Rₑ is independently alkyl, cycloalkyl, alkenyl, cycloalkenyl, alkynyl, heterocycle, or aryl. In some embodiments, groups such as alkyl, cycloalkyl, alkenyl, alkynyl, cycloalkenyl, heterocycle, and aryl can themselves be optionally substituted.

The term “heteroalkyl” refers to a straight- or branched-chain alkyl group preferably having from 2 to 12 carbons, more preferably 2 to 10 carbons in the chain, one or more of which has been replaced by a heteroatom selected from the group consisting of S, O, P, and N. Exemplary heteroalkyls include, but are not limited to, alkyl ethers, secondary and tertiary alkyl amines, alkyl sulfides, and the like. The group may be a terminal group or a bridging group. In some embodiments, heteroalkyl is optionally substituted.

The term “alkenyl” refers to a straight or branched chain hydrocarbon radical containing from 2 to 12 carbon atoms and at least one carbon-carbon double bond. Exemplary such groups include ethenyl or allyl. The term “C₂-C₆ alkenyl” refers to a straight or branched chain hydrocarbon radical containing from 2 to 6 carbon atoms and at least one carbon-carbon double bond, such as ethylenyl, propenyl, 2-propenyl, (E)-but-2-enyl, (Z)-but-2-enyl, 2-methy (E)-but-2-enyl, 2-methy (Z)-but-2-enyl, 2,3-dimethy-but-2-enyl, (Z)-pent-2-enyl, (E)-pent-1-enyl, (Z)-hex-1-enyl, (E)-pent-2-enyl, (Z)-hex-2-enyl, (E)-hex-2-enyl, (Z)-hex-1-enyl, (E)-hex-1-enyl, (Z)-hex-3-enyl, (E)-hex-3-enyl, and (E)-hex-1,3-dienyl. “Substituted alkenyl” refers to an alkenyl group substituted with one or more substituents, preferably 1 to 4 substituents, at any available point of attachment. Exemplary substituents include, but are not limited to, one or more of the following groups: hydrogen, halogen, alkyl, halogenated alkyl (i.e., an alkyl group bearing a single halogen substituent or multiple halogen substituents such as CF₃ or CCl₃), cyano, nitro, oxo (i.e., ═O), CF₃, OCF₃, cycloalkyl, bicycloalkyl, spiroalkyl, alkenyl, cycloalkenyl, alkynyl, heterocycle, aryl, ORₐ, SRₐ, S(═O)Rₑ, S(═O)₂Rₑ, —N═S(═O)(Rₐ), —RₐS(═O)(═NRₐ), S(═O)(═NRₐ)(═N(Rₐ)₂) (linked to the molecule via Rₐ or N), P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NRₐS(═O)₂NR_{b}R_{c}, NRₐP(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and Rₐ is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “alkynyl” refers to a straight or branched chain hydrocarbon radical containing from 2 to 12 carbon atoms and at least one carbon to carbon triple bond. Exemplary groups include ethynyl. The term “C₂-C₆ alkynyl” refers to a straight or branched chain hydrocarbon radical containing from 2 to 6 carbon atoms and at least one carbon-carbon triple bond, such as ethynyl, prop-1-ynyl, prop-2-ynyl, but-1-ynyl, but-2-ynyl, pent-1-ynyl, pent-2-ynyl, hex-1-ynyl, hex-2-ynyl, or hex-3-ynyl. “Substituted alkynyl” refers to alkynyl substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, bicycloalkyl, spiroalkyl, alkenyl, cycloalkenyl, alkynyl, heterocycle, aryl, ORₐ, SRₐ, S(═O)Rₑ, S(═O)₂Rₑ, P(═O)₂R_{c}, S(═O)₂ORₑ, —N═S(═O)(Rₐ), —RₐS(═O)(═NRₐ), S(═O)(═NRₐ)(═N(Rₐ)₂) (linked to the molecule via Rₐ or N), P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NRₐS(═O)₂NR_{b}R_{c}, NRₐP(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and Rₐ is independently hydrogen, alkyl, cycloalkyl, heterocycle, aryl, or said R_{b} and R_{c} together with the N to which they are bonded optionally to form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “cycloalkyl” refers to a fully saturated cyclic hydrocarbon group containing from 1 to 4 rings and 3 to 8 carbons per ring. “C₃-C₇ cycloalkyl” refers to cyclopropyl, cyclobutyl, cyclopentyl, cyclohexyl, or cycloheptyl. “Substituted cycloalkyl” refers to a cycloalkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, bicycloalkyl, spiroalkyl, alkenyl, cycloalkenyl, alkynyl, heterocycle, aryl, ORₐ, SRₐ, S(═O)Rₑ, S(═O)₂Rₑ, —N═S(═O)(Rₐ), —RₐS(═O)(═NRₐ), S(═O)(═NRₐ)(═N(Rₐ)₂) (linked to the molecule via Rₐ or N), P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NRₐS(═O)₂NR_{b}R_{c}, NRₐP(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and Rₐ is independently hydrogen, alkyl, cycloalkyl, heterocycle, aryl, or said R_{b} and R_{c} together with the N to which they are bonded optionally to form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned substituents, such as cycloalkyl, cycloalkenyl, heterocycle and aryl substituents, can themselves be optionally substituted.

The term “bicycloalkyl” or “spiroalkyl” refers to a group containing at least one cycloalkyl ring that shares one or more ring atoms with at least one other cycloalkyl ring. The term “heterobicycloalkyl” or “heterospiroalkyl” refers to a bicycloalkyl group in which at least one, preferably from 1-3, carbon atoms in at least one ring are replaced with a heteroatom selected from the group consisting of N, S, O, or P. The heteroatom may occupy a terminal position or a bridging position (i.e., a connection point between two rings). Exemplary bicycloalkyl groups include adamantyl, bicyclo[1.1.1]pentyl, bicyclo[2.2.1]heptyl, bicyclo[3.1.1]heptyl, bicyclo[2.1.1]hexyl, octahydropentalenyl, bicyclo[3.2.1]octyl, bicyclo[3.3.3]undecanyl, decahydronaphthalenyl, bicyclo[3.2.0]heptyl, octahydro-1H-indenyl, bicyclo[4.2.1]nonanyl, and the like. Exemplary spiro bicycloalkyl groups include spiro[4.4]nonyl, spiro[3.3]heptyl, spiro[5.5]undecyl, spiro[3.5]nonyl, spiro[4.5]decyl, and the like. “Substituted bicycloalkyl”, “substituted spiroalkyl”, “substituted heterobicycloalkyl”, and “substituted heterospiroalkyl” refer to a bicycloalkyl, spiroalkyl, heterobicycloalkyl, or heterospiroalkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, bicycloalkyl, spiroalkyl, alkenyl, cycloalkenyl, alkynyl, heterocycle, aryl, ORₐ, SRₐ, S(═O)Rₑ, S(═O)₂Rₑ, —N═S(═O)(Rₐ), —RₐS(═O)(═NRₐ), S(═O)(═NRₐ)(═N(Rₐ)₂) (linked to the molecule via Rₐ or N), P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NRₐS(═O)₂NR_{b}R_{c}, NRₐP(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and Rₐ is independently hydrogen, alkyl, cycloalkyl, heterocycle, aryl, or said R_{b} and R_{c} together with the N to which they are bonded optionally to form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned substituents, such as cycloalkyl, cycloalkenyl, heterocycle and aryl substituents, can themselves be optionally substituted.

The term “heterocycloalkyl” or “cycloheteroalkyl” refers to a saturated or partially saturated monocyclic, bicyclic, or polycyclic ring containing at least one heteroatom selected from the group consisting of nitrogen, sulfur, and oxygen, preferably from 1 to 3 heteroatoms in at least one ring. Each ring is preferably from 3 to 10 membered, more preferably 4 to 7 membered. In some embodiments, heterocycloalkyl or cycloheteroalkyl is optionally substituted. Examples of suitable heterocycloalkyl substituents include, but are not limited to, azetidinyl, oxetanyl, pyrrolidyl, tetrahydrofuryl, tetrahydrothiofuranyl, piperidyl, piperazyl, tetrahydropyranyl, morpholino, 1,3-diazepanyl, 1,4-diazepanyl, 1,4-oxazepanyl, and 1,4-oxathiapanyl. The group may be a terminal group or a bridging group.

The term “cycloalkenyl” refers to a partially unsaturated cyclic hydrocarbon group containing 1 to 4 rings and 3 to 8 carbons per ring. Exemplary such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, bicycloalkyl, spiroalkyl, alkenyl, cycloalkenyl, alkynyl, heterocycle, aryl, ORₐ, SRₐ, S(═O)Rₑ, S(═O)₂Rₑ, —N═S(═O)(Rₐ), —RₐS(═O)(═NRₐ), S(═O)(═NRₐ)(═N(Rₐ)₂) (linked to the molecule via Rₐ or N), P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NRₐS(═O)₂NR_{b}R_{c}, NRₐP(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c}, and Rₐ is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned substituents, such as cycloalkyl, cycloalkenyl, heterocycle and aryl substituents, can themselves be optionally substituted.

The term “aryl” refers to cyclic, aromatic hydrocarbon groups that have 1 to 5 aromatic rings, especially monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). The term “fused aromatic ring” refers to a molecular structure having two or more aromatic rings where two adjacent aromatic rings have two carbon atoms in common. “Substituted aryl” refers to an aryl group substituted by one or more substituents, preferably 1 to 3 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, bicycloalkyl, spiroalkyl, alkenyl, cycloalkenyl, alkynyl, heterocycle, aryl, ORₐ, SRₐ, S(═O)Rₑ, S(═O)₂Rₑ, —N—S(═O)(Rₐ), —RₐS(═O)(═NRₐ), S(═O)(═NRₐ)(═N(Rₐ)₂) (linked to the molecule via Rₐ or N), P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NRₐS(═O)₂NR_{b}R_{c}, NRₐP(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and Rₐ is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include fused cyclic groups, especially fused cycloalkyl, fused cycloalkenyl, fused heterocycle, or fused aryl, where the aforementioned substituents, such as cycloalkyl, cycloalkenyl, heterocycle, and aryl, substituents can themselves be optionally substituted.

The term “biaryl” refers to two aryl groups linked by a single bond. The term “biheteroaryl” refers to two heteroaryl groups linked by a single bond. Similarly, the term “heteroaryl-aryl” refers to a heteroaryl group and an aryl group linked by a single bond and the term “aryl-heteroaryl” refers to an aryl group and a heteroaryl group linked by a single bond. In certain embodiments, the numbers of the ring atoms in the heteroaryl and/or aryl rings are used to specify the sizes of the aryl or heteroaryl ring in the substituents. For example, 5,6-heteroaryl-aryl refers to a substituent in which a 5-membered heteroaryl is linked to a 6-membered aryl group. Other combinations and ring sizes can be similarly specified.

The term “carbocycle” or “carbon cycle” refers to a fully saturated or partially saturated cyclic hydrocarbon group containing from 1 to 4 rings and 3 to 8 carbons per ring, or cyclic, aromatic hydrocarbon groups that have 1 to 5 aromatic rings, especially monocyclic or bicyclic groups such as phenyl, biphenyl, or naphthyl. The term “carbocycle” encompasses cycloalkyl, cycloalkenyl, cycloalkynyl, and aryl as defined hereinabove. The term “substituted carbocycle” refers to carbocycle or carbocyclic groups substituted with one or more substituents, preferably 1 to 4 substituents, at any available point of attachment. Exemplary substituents include, but are not limited to, those described above for substituted cycloalkyl, substituted cycloalkenyl, substituted cycloalkynyl, and substituted aryl. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned substituents, such as cycloalkyl, cycloalkenyl, heterocycle, and aryl substituents, can themselves be optionally substituted.

The terms “heterocycle” and “heterocyclic” refer to fully saturated, or partially or fully unsaturated, including aromatic (i.e., “heteroaryl”) cyclic groups (for example, 3 to 7 membered monocyclic, 7 to 11 membered bicyclic, or 8 to 16 membered tricyclic ring systems) which have at least one heteroatom in at least one carbon atom-containing ring. Each ring of the heterocyclic group may independently be saturated, or partially or fully unsaturated. Each ring of the heterocyclic group containing a heteroatom may have 1, 2, 3, or 4 heteroatoms selected from the group consisting of nitrogen atoms, oxygen atoms and sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, indolinyl, isoindolyl, benzothiazolyl, benzoxazolyl, benzoxadiazolyl, benzothienyl, benzo[d][1,3]dioxolyl, dihydro-2H-benzo[b][1,4]oxazine, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, dihydrobenzo[d]oxazole, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl, and the like. Exemplary tricyclic heterocyclic groups include carbazolyl, benzidolyl, phenanthrolinyl, acridinyl, phenanthridinyl, xanthenyl, and the like. The term “partially saturated bicyclic heteroaryl” refers to a bicyclic heteroaryl that is partially saturated, e.g., having a saturated cycloalkyl or heterocyclic alkyl ring.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, bicycloalkyl, spiroalkyl, alkenyl, cycloalkenyl, alkynyl, heterocycle, aryl, ORₐ, SRₐ, S(═O)Rₑ, S(═O)₂Rₑ, —N═S(═O)(Rₐ), —RₐS(═O)(═NRₐ), S(═O)(═NRₐ)(═N(Rₐ)₂) (linked to the molecule via Rₐ or N), P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NRₐS(═O)₂NR_{b}R_{c}, NRₐP(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and Rₐ is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned substituents, such as cycloalkyl, cycloalkenyl, heterocycle and aryl substituents, can themselves be optionally substituted.

The term “oxo” refers to

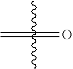 [SMILES: *=O]

substituent group, which may be attached to a carbon ring atom on a carboncycle or heterocycle. When an oxo substituent group is attached to a carbon ring atom on an aromatic group, e.g., aryl or heteroaryl, the bonds on the aromatic ring may be rearranged to satisfy the valence requirement. For instance, a pyridine with a 2-oxo substituent group may have the structure of

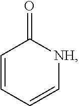

which also includes its tautomeric form of

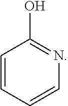

The term “alkylamino” refers to a group having the structure —NHR′, where R′ is hydrogen, alkyl or substituted alkyl, cycloalkyl or substituted cycloalkyl, as defined herein. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′, where R and R′ are each independently alkyl or substituted alkyl, cycloalkyl or substituted cycloalkyl, cycloalkenyl or substituted cyclolalkenyl, aryl or substituted aryl, heterocycle or substituted heterocycle, as defined herein. R and R′ may be the same or different in a dialkyamino moiety. Examples of dialkylamino groups include, but are not limited to, dimethylamino, methyl ethylamino, diethylamino, methylpropylamino, di(n-propyl)amino, di(isopropyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of the resulting cyclic structure include, but are not limited to, aziridinyl, pyrrolidinyl, piperidinyl, morpholinyl, pyrrolyl, imidazolyl, 1,2,4-triazolyl, and tetrazolyl.

The terms “halogen” or “halo” refer to chlorine, bromine, fluorine, or iodine.

The term “substituted” refers to the embodiments in which a molecule, molecular moiety, or substituent group (e.g., alkyl, cycloalkyl, alkenyl, cycloalkenyl, alkynyl, heterocycle, or aryl group or any other group disclosed herein) is substituted with one or more substituents, where valence permits, preferably 1 to 6 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, alkyl, halogen-substituted alkyl, cycloalkyl, bicycloalkyl, spiroalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, —N═S(═O)(Rₐ), —RₐS(═O)(═NRₐ), S(═O)(═NRₐ)(═N(Rₐ)₂) (linked to the molecule via Rₐ or N), P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NRₐS(═O)₂NR_{b}R_{c}, NRₐP(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and Rₐ is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. In the aforementioned exemplary substituents, groups such as alkyl, cycloalkyl, alkenyl, alkynyl, cycloalkenyl, heterocycle, and aryl can themselves be optionally substituted. The term “optionally substituted” refers to the embodiments in which a molecule, molecular moiety or substituent group (e.g., alkyl, cycloalkyl, alkenyl, cycloalkenyl, alkynyl, heterocycle, or aryl group or any other group disclosed herein) may or may not be substituted with aforementioned one or more substituents.

Unless otherwise indicated, any heteroatom with unsatisfied valences is assumed to have hydrogen atoms sufficient to satisfy the valences.

The compounds of the present invention may form salts which are also within the scope of this invention. Reference to a compound of the present invention is understood to include reference to salts thereof, unless otherwise indicated. The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically-acceptable (i.e., non-toxic, physiologically-acceptable) salts are preferred, although other salts are also useful, e.g., in isolation or purification steps which may be employed during preparation. Salts of the compounds of the present invention may be formed, for example, by reacting a compound described herein with an amount of acid or base, such as an equivalent amount, in a medium such as one in which the salt precipitates, or in an aqueous medium followed by lyophilization.

The compounds of the present invention which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid; for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of the present invention which contain an acidic moiety, such as but not limited to a carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glycamides, t-butyl amines, and salts with amino acids such as arginine, lysine, and the like. Basic nitrogen-containing groups may be quaternized with agents such as lower alkyl halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl and stearyl chlorides, bromides, and iodides), aralkyl halides (e.g., benzyl and phenethyl bromides), and others.

Prodrugs and solvates of the compounds of the invention are also contemplated herein. The term “prodrug” as employed herein denotes a compound that, upon administration to a subject, undergoes chemical conversion by metabolic or chemical processes to yield a compound of the present invention, or a salt and/or solvate thereof. Solvates of the compounds of the present invention include, for example, hydrates.

Compounds of the present invention, and salts or solvates thereof, may exist in their tautomeric form (for example, as an amide or iminol). All such tautomeric forms are contemplated herein as part of the present invention. As used herein, any depicted structure of the compound includes the tautomeric forms thereof.

All stereoisomers of the compounds described herein (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives, or separation by chiral column chromatography. The individual optical isomers can be obtained from the racemates by any suitable method, including without limitation, conventional methods, such as, for example, salt formation with an optically active acid followed by crystallization.

Compounds of the present invention are, subsequent to their preparation, preferably isolated and purified to obtain a composition containing an amount by weight equal to or greater than 90%, for example, equal to or greater than 95%, equal to or greater than 99% of the compounds (“substantially pure” compounds), which is then used or formulated as described herein. Such “substantially pure” compounds of the present invention are also contemplated herein as part of the present invention.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

Throughout the specification, groups and substituents thereof may be chosen to provide stable moieties and compounds.

Definitions of specific functional groups and chemical terms are described in more detail herein. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito (1999), the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

The present invention also includes isotopically labeled compounds, which are identical to the compounds disclosed herein, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine, and chlorine, such as ²H (or D), ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, or an enantiomer, diastereomer, tautomer, or pharmaceutically-acceptable salt or solvate thereof, which contain the aforementioned isotopes and/or other isotopes of other atoms are within the scope of this invention. Certain isotopically labeled compounds of the present invention, for example, those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example, increased in vivo half-life or reduced dosage requirements, and hence may be preferred in some circumstances. Isotopically-labeled compounds can generally be prepared by carrying out the procedures disclosed in the Schemes and/or in the Examples below, by substituting a readily-available isotopically-labeled reagent for a non-isotopically-labeled reagent.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valences of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of proliferative disorders. The term “stable,” as used herein, preferably refers to compounds which possess stability sufficient to allow manufacture and which maintain the integrity of the compound for a sufficient period of time to be detected and preferably for a sufficient period of time to be useful for the purposes detailed herein.

As used herein, the terms “cancer” and, equivalently, “tumor” refer to a condition in which abnormally replicating cells of host origin are present in a detectable amount in a subject. The cancer can be a malignant or non-malignant cancer. Cancers or tumors include, but are not limited to, adult T-cell leukemia/lymphoma (including that caused by human T-cell lymphotropic virus (HTLV-1)), biliary tract cancer; brain cancer; breast cancer; cervical cancer; choriocarcinoma; colon cancer; endometrial cancer; esophageal cancer; gastric (stomach) cancer; intraepithelial neoplasms; leukemias; lymphomas; liver cancer; lung cancer (e.g., small cell and non-small cell); melanoma; neuroblastomas; oral cancer; ovarian cancer; pancreatic cancer; prostate cancer; rectal cancer; renal (kidney) cancer; sarcomas; skin cancer; testicular cancer; thyroid cancer; as well as other carcinomas and sarcomas. As used herein, the term “lymphoma” refers to cancer of the lymphatic system or a blood cancer that develops from lymphocytes. Cancers can be primary or metastatic. Diseases other than cancers may be associated with mutational alternation of component of Ras signaling pathways and the compound disclosed herein may be used to treat these non-cancer diseases. Such non-cancer diseases may include: neurofibromatosis; Leopard syndrome; Noonan syndrome; Legius syndrome; Costello syndrome; cardio-facio-cutaneous syndrome; hereditary gingival fibromatosis type 1; autoimmune lymphoproliferative syndrome; and capillary malformation-arterovenous malformation.

As used herein, “effective amount” refers to any amount that is necessary or sufficient for achieving or promoting a desired outcome. In some instances, an effective amount is a therapeutically effective amount. A therapeutically effective amount is any amount that is necessary or sufficient for promoting or achieving a desired biological response in a subject. The effective amount for any particular application can vary depending on such factors as the disease or condition being treated, the particular agent being administered, the size of the subject, or the severity of the disease or condition. One of ordinary skill in the art can empirically determine the effective amount of a particular agent without necessitating undue experimentation.

As used herein, the term “subject” refers to a vertebrate animal. In one embodiment, the subject is a mammal or a mammalian species. In one embodiment, the subject is a human. In other embodiments, the subject is a non-human vertebrate animal, including, without limitation, non-human primates, laboratory animals, livestock, racehorses, domesticated animals, and non-domesticated animals.

The term “immune cell” as used herein refers to cells of the innate and acquired immune system including, but not limited to, neutrophils, eosinophils, basophils, glial cells (e.g., astrocytes, microglia, and oligodendrocytes), monocytes, macrophages, dendritic cells, lymphocytes including B cells, T cells, and NK cells.

As used herein, “conventional T cells” are T lymphocytes that express an αβ T cell receptor (“TCR”) as well as a co-receptor CD4 or CD8. Conventional T cells are present in the peripheral blood, lymph nodes, and tissues. See Roberts and Girardi, “Conventional and Unconventional T Cells”, Clinical and Basic Immunodermatology, pp. 85-104, (Gaspari and Tyring (ed.)), Springer London (2008), herein incorporated by reference in its entirety. As used herein, “unconventional T cells” are lymphocytes that express a γδ TCR and may commonly reside in an epithelial environment, such as the skin, gastrointestinal tract, or genitourinary tract. Another subset of unconventional T cells is the invariant natural killer T (“NKT”) cell, which has phenotypic and functional capacities of a conventional T cell, as well as features of natural killer cells (e.g., cytolytic activity). See id. As used herein, regulatory T cells (“Tregs”) are a subpopulation of T cells which modulate the immune system, maintain tolerance to self-antigens, abrogate autoimmune disease, and otherwise suppress immune-stimulating or activating responses of other cells. Tregs come in many forms, with the most well-understood being those that express CD4, CD25, and Foxp3. As used herein, “natural Treg” or “nTreg” refer to a Treg or cells that develop in the thymus. As used herein, “induced Treg” or “iTreg” refer to a Treg or cells that develop from mature CD4+ conventional T cells outside of the thymus.

The “activity” of Akt3 refers to the biological function of the Akt3 protein. Bioactivity can be increased or reduced by increasing or reducing the activity of basal levels of the protein, increasing or reducing the avidity of basal levels of the protein, the quantity of the protein, the ratio of Akt3 relative to one or more other isoforms of Akt (e.g., Akt1 or Akt2) protein, increasing or reducing the expression levels of the protein (including by increasing or decreasing mRNA expression of Akt3), or a combination thereof. For example, bioavailable Akt3 protein is a protein that has kinase activity and can bind to and phosphorylate a substrate of Akt3. Akt3 protein that is not bioavailable includes Akt3 protein that is mis-localized or incapable of binding to and phosphorylating Akt substrates.

In some embodiments, the disclosed compounds selectively modulate Akt3 compared to Akt1 and Akt2. In some embodiments, any one of the disclosed compounds do not modulate Akt1 and Akt2 to a statistically significant degree. In other embodiments, modulation of Akt3 by the disclosed compounds is about 5, about 10, about 15, about 50, about 100, about 1000, or about 5000-fold greater than their modulations of Akt1 and/or Akt2.

As used herein, the term “peptide” or “polypeptide” refers to a chain of amino acids of any length, regardless of modification (e.g., phosphorylation or glycosylation). The terms include proteins and fragments thereof. The polypeptides can be “exogenous,” meaning that they are “heterologous,” i.e., foreign to the host cell being utilized, such as human polypeptide produced by a bacterial cell. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: alanine (Ala, A), arginine (Arg, R), asparagine (Asn, N), aspartic Acid (Asp, D), cysteine (Cys, C), glutamine (Gln, Q), glutamic Acid (Glu, E), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V).

The term “stimulate expression of” means to affect expression of, for example, to induce expression or activity, or induce increased/greater expression or activity relative to normal, healthy controls.

The terms “immune activating response”, “activating immune response”, and “immune stimulating response” refer to a response that initiates, induces, enhances, or increases the activation or efficiency of innate or adaptive immunity. Such immune responses include, for example, the development of a beneficial humoral (antibody-mediated) and/or a cellular (mediated by antigen-specific T cells or their secretion products) response directed against a peptide in a recipient patient. Such a response can be an active response, induced by administration of immunogen, or a passive response, induced by administration of antibody or primed T-cells. A cellular immune response is elicited by the presentation of polypeptide epitopes in association with class I or class II major histocompatibility complex (“MHC”) molecules to activate antigen-specific CD4+ T helper cells and/or CD8+ cytotoxic T cells. The response can also involve activation of monocytes, macrophages, NK cells, basophils, dendritic cells, astrocytes, microglia cells, eosinophils, activation or recruitment of neutrophils, or other components of innate immunity. The presence of a cell-mediated immunological response can be determined by proliferation assays (CD4+ T cells) or cytotoxic T lymphocyte (“CTL”) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

The terms “suppressive immune response” and “immune suppressive response” refer to a response that reduces or prevents the activation or efficiency of innate or adaptive immunity.

The term “immune tolerance” refers to any mechanism by which a potentially injurious immune response is prevented, suppressed, or shifted to a non-injurious immune response (see Bach, et al., N. Eng. J. Med., 347:911-920 (2002)).

The terms “immunogenic agent” or “immunogen” refer to an agent capable of inducing an immunological response against itself on administration to a mammal, optionally in conjunction with an adjuvant.

### Compounds

In one aspect, a compound of Formula Ia, Ib, or Ic as an Akt3 modulator is described. Applicants have surprisingly discovered that the compounds disclosed herein modulate Akt3 activity, e.g., activate or inhibit Akt3 activity, and/or a downstream event, depending on the structure and substitutions thereof.

In one aspect, a compound of Formula Ia, Ib, or Ic is described,

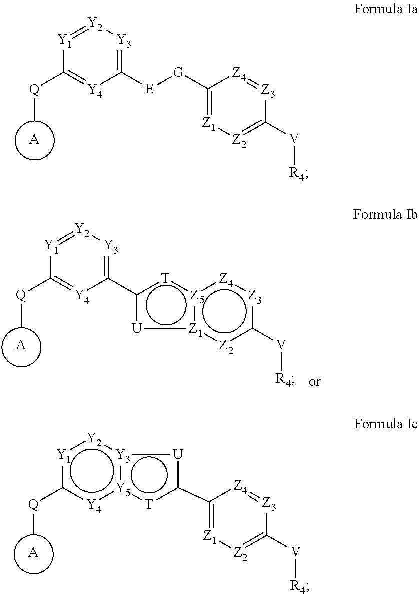

or a pharmaceutically acceptable salt thereof,
where:
   \textcircled{A} is

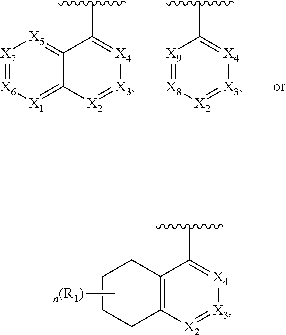

each occurrence of X₁, X₂, X₃, X₄, X₅, X₆, X₇, X₈, and X₉ is independently CR₁ or N;
each occurrence of R₁ is independently selected from the group consisting of H, D, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₇)cycloalkyl, (C₄-C₁₀)bicycloalkyl, (C₄-C₁₄)tricycloalkyl, (C₃-C₇)heterocycloalkyl, halogenated (C₃-C₇)heterocycloalkyl, (C₄-C₁₀)heterobicycloalkyl, (C₄-C₁₄)heterotricycloalkyl, (C₄-C₁₀)heterospiroalkyl, (C₃-C₇)cycloalkenyl, (C₃-C₇)heterocycloalkenyl, (C₄-C₁₀)bicycloalkenyl, (C₄-C₁₀)heterobicycloalkenyl, (C₄-C₁₄)tricycloalkenyl, (C₄-C₁₄)heterotricycloalkenyl, aryl, heteroaryl, —ORₐ, —SRₐ, —N(Rₐ)₂, —CORₐ, —CO₂Rₐ, CON(Rₐ)₂, —CN, —NC, NO₂, N₃, —SO₂Rₐ, —SO₂N(Rₐ)₂, —N(Rₐ)SO₂Rₐ,

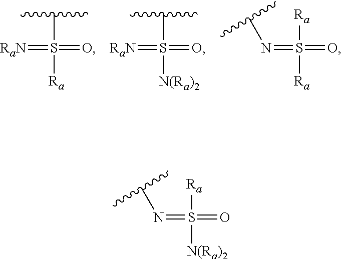

and a partially saturated bicyclic heteroaryl optionally substituted by one or more (C₁-C₆)alkyl, halogenated (C₁-C₆)alkyl, —SO₂Rₐ, or —SO₂N(Rₐ)₂;
wherein the (C₃-C₇)cycloalkyl, (C₄-C₁₀)bicycloalkyl, (C₄-C₁₄)tricycloalkyl, (C₃-C₇)heterocycloalkyl, halogenated (C₃-C₇)heterocycloalkyl, (C₄-C₁₀)heterobicycloalkyl, (C₄-C₁₄)heterotricycloalkyl, (C₄-C₁₀)heterospiroalkyl, (C₃-C₇)cycloalkenyl, (C₃-C₇)heterocycloalkenyl, (C₄-C₁₀)bicycloalkenyl, (C₄-C₁₀)heterobicycloalkenyl, (C₄-C₁₄)tricycloalkenyl, (C₄-C₁₄)heterotricycloalkenyl, aryl, and heteroaryl of R₁ are each optionally substituted by one or more (C₁-C₆)alkyl, halogenated (C₁-C₆)alkyl, halogen, —ORₐ, —CN, or —N(Rₐ)₂;
n is an integer from 0-4 where valence permits;
Q is C(Rₐ)₂, O, NRₐ, N(C═O)Rₐ, or NSO₂Rₐ;
Y₁, Y₂, Y₃, Y₄ and Y₅ are each independently N or CR₂ where valance permits;
R₂ is selected from the group consisting of H, D, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₇)cycloalkyl, (C₄-C₁₀)bicycloalkyl, (C₃-C₇)heterocycloalkyl, (C₄-C₁₀)heterobicycloalkyl, (C₄-C₁₀)heterospiroalkyl, halogenated (C₃-C₇)heterocycloalkyl, aryl, heteroaryl, —ORₐ, —SRₐ, N(Rₐ)₂, —CORₐ, —CO₂Rₐ, CON(Rₐ)₂, —CN, —NC, NO₂, N₃, —SO₂Rₐ, —SO₂N(Rₐ)₂, —N(Rₐ)SO₂Rₐ,

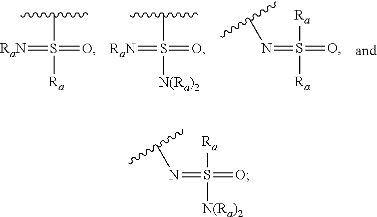

-E-G- is —(C═O)NRₓ—, —NRₓ(C═O)—, —N(Rₓ)(C═O)N(Rₓ)—, —O(C═O)N(Rₓ)—, —N(Rₓ)(C═O)O—, —SO₂NRₓ—, —NRₓSO₂—, or

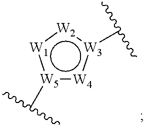

where
each occurrence of Rₓ is independently H, (C₁-C₆)alkyl, (C₃-C₇)cycloalkyl, aryl, or heteroaryl; or wherein Rₓ and Y₃, Rₓ and Y₄, Rₓ and Z₁, or Rₓ and Z₄ taken together form a 5-6-membered heterocycle optionally substituted by one or more R_{z}, wherein each occurrence of R_{z} is independently H, (C₁-C₆)alkyl, OH, O(C₁-C₆)alkyl, or halogen; or two R_{z} taken together form a 4-6-membered ring optionally substituted with halogen or (C₁-C₆)alkyl;
W₁, W₂, W₃, W₄, and W₅ are each independently CR₆, N, or NR₆ where valence permits;
each occurrence of R₆ is independently selected from the group consisting of H, halogen, (C₁-C₆)alkyl, and (C₁-C₆)haloalkyl;
each occurrence of T is independently O, N, NRₐ, N(C═O)Rₐ, NC(R_{b})₂OP(═O)(OR_{b})₂, or NSO₂Rₐ where valance permits;
each occurrence of U is independently O, N, NRₐ, N(C═O)Rₐ, NC(R_{b})₂OP(═O)(OR_{b})₂, or NSO₂Rₐ where valance permits;
each occurrence of R_{b} is independently H or (C₁-C₆)alkyl;
Z₁, Z₂, Z₃, Z₄ and Z₅ are each independently N or CR₃ where valance permits;
R₃ is selected from the group consisting of H, D, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₇)cycloalkyl, (C₄-C₁₀)bicycloalkyl, (C₃-C₇)heterocycloalkyl, (C₄-C₁₀)heterobicycloalkyl, (C₄-C₁₀)heterospiroalkyl, halogenated (C₃-C₇)heterocycloalkyl, aryl, heteroaryl, —ORₐ, —SRₐ, —N(Rₐ)₂, —CORₐ, —CO₂Rₐ, CON(Rₐ)₂, —CN, —NC, NO₂, N₃, —SO₂Rₐ, —SO₂N(Rₐ)₂, —N(Rₐ)SO₂Rₐ,

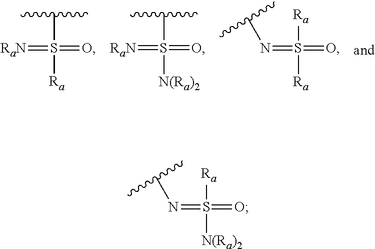

V is absent, C(Rₐ)₂, NRₐ, N(C═O)Rₐ, NSO₂Rₐ or O;
R₄ is selected from the group consisting of (C₁-C₆)alkyl, (C₃-C₇)cycloalkyl, (C₄-C₁₀)bicycloalkyl, (C₃-C₇)heterocycloalkyl, (C₄-C₁₀)heterobicycloalkyl, (C₄-C₁₀)heterospiroalkyl, aryl, heteroaryl, each optionally substituted with one or more R₅;
or alternatively V and R₄ taken together form a (C₃-C₇)heterocycloalkyl or (C₄-C₁₀)heterospiroalkyl;
each occurrence of R₅ is independently selected from the group consisting of H, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₇)cycloalkyl, (C₄-C₁₀)bicycloalkyl, (C₃-C₇)heterocycloalkyl, (C₄-C₁₀)heterobicycloalkyl, (C₄-C₁₀)heterospiroalkyl, halogenated (C₃-C₇)heterocycloalkyl, aryl, heteroaryl, —ORₐ, —SRₐ, —N(Rₐ)₂, —CORₐ, —CO₂Rₐ, CON(Rₐ)₂, —CN, —NC, NO₂, N₃, —SO₂Rₐ, —SO₂N(Rₐ)₂, —N(Rₐ)SO₂Rₐ, N(Rₐ)CORₐ,

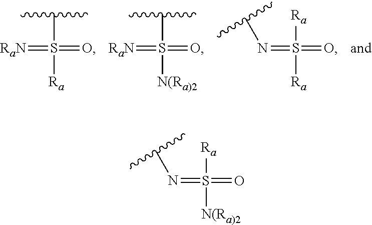

each occurrence of Rₐ is independently H, (C₁-C₆)alkyl, (C₂-C₆)alkenyl, (C₃-C₇)cycloalkyl, aryl, or heteroaryl, or two Rₐ taken together form a 4-6-membered ring optionally substituted with halogen or (C₁-C₆)alkyl.

In some embodiments, \textcircled{A} is

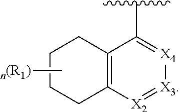

In some embodiments, \textcircled{A} is

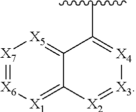

In some embodiments, \textcircled{A} is

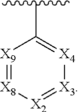

In some embodiments, Q is C(Rₐ)₂, O, or NRₐ. In some embodiments, Q is O. In some embodiments, Q is NRₐ. In some embodiments, Q is NH. In some embodiments, Q is NCH₃ or NCH₂CH₃. In some embodiments, Q is N(C═O)Rₐ or NSO₂Rₐ. In some embodiments, Q is N(C—O)H. In some embodiments, Q is N(C═O)CH₃ or N(C═O)CH₂CH₃. In some embodiments, Q is NSO₂H. In some embodiments, Q is NSO₂CH₃ or NSO₂CH₂CH₃.

In some embodiments, n is 0, 1, 2, 3, or 4. In some embodiments, n is 0. In some embodiments, n is 1. In some embodiments, n is 2. In some embodiments, n is 3. In some embodiments, n is 4.

In some embodiments, \textcircled{A} is

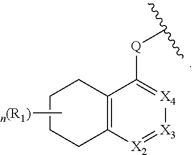

In some embodiments, X₂, X₃, and X₄ are each independently CR₁ or N. In some embodiments, X₂, X₃, and X₄ are CR₁. In some embodiments, X₂, X₃, and X₄ are CH. In some embodiments, one of X₂, X₃, and X₄ is N and the rest are CR₁. In some embodiments, one of X₂, X₃, and X₄ is N and the rest are CH. In some embodiments, two of X₂, X₃, and X₄ are N and the rest are CR₁. In some embodiments, two of X₂, X₃, and X₄ are N and the rest are CH.

In some embodiments, the structural moiety

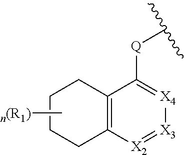

has the structure of

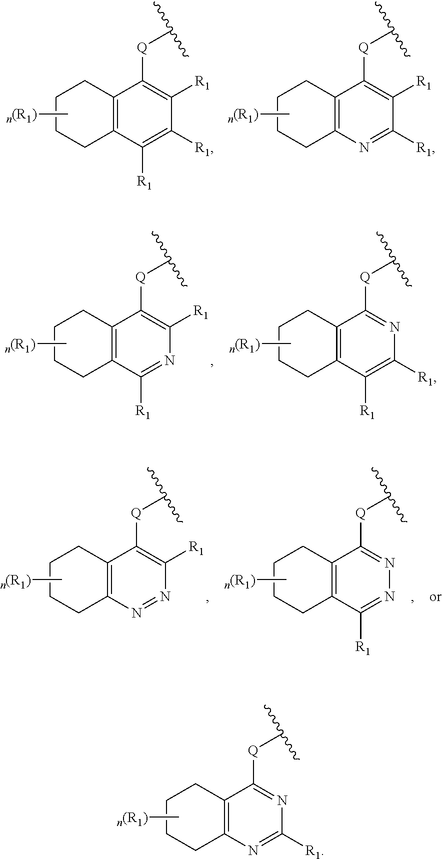

In some embodiments, the structural moiety

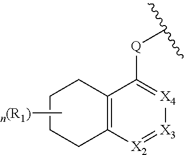

has the structure of

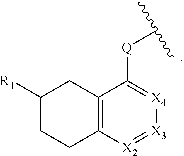

In some embodiments, the structural moiety

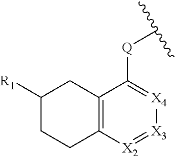

has the structure of

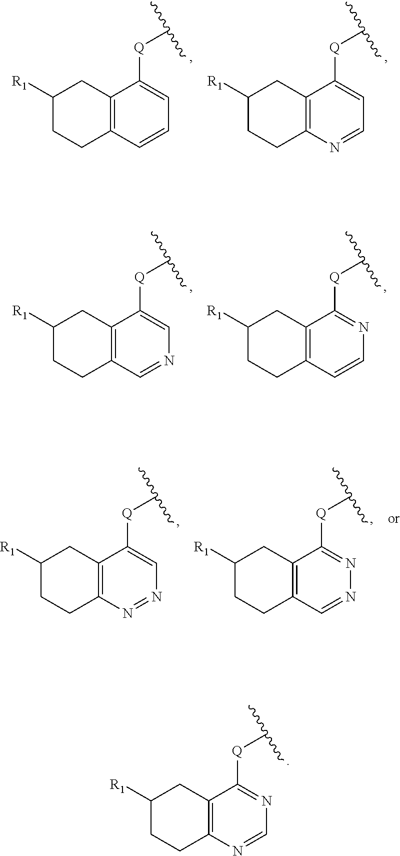

In some embodiments, X₁, X₂, X₃, X₄, X₅, X₆, and X₇ are each independently CR₁ or N. In some embodiments, X₁, X₂, X₃, X₄, X₅, X₆, and X₇ are CR₁. In some embodiments, X₁, X₂, X₃, X₄, X₅, X₆, and X₇ are each independently CH, C (halogen), or CCH₃. In some embodiments, one of X₁, X₂, X₃, X₄, X₅, X₆, and X₇ is N and the rest are CR₁. In some embodiments, one of X₁, X₂, X₃, X₄, X₅, X₆, and X₇ is N and the rest are each independently CH, C (halogen), or CCH₃. In some embodiments, two of X₁, X₂, X₃, X₄, X₅, X₆, and X₇ are N and the rest are CR₁. In some embodiments, two of X₁, X₂, X₃, X₄, X₅, X₆, and X₇ are N and the rest are each independently CH, C (halogen), or CCH₃. In some embodiments, three of X₁, X₂, X₃, X₄, X₅, X₆, and X₇ are N and the rest are CR₁. In some embodiments, three of X₁, X₂, X₃, X₄, X₅, X₆, and X₇ are N and the rest are each independently CH, C (halogen), or CCH₃. In some embodiments, four of X₁, X₂, X₃, X₄, X₅, X₆, and X₇ are N and the rest are CR₁. In some embodiments, four of X₁, X₂, X₃, X₄, X₅, X₆, and X₇ are N and the rest are each independently CH, C (halogen), or CCH₃. In some embodiments, X₂ is N, X₇ is CR₁, and X₁, X₃, X₄, X₅, and X₆ are each independently CH, C (halogen), or CCH₃. In some embodiments, X₂ is N, X₇ is CR₁, X₃ is C (halogen) or CCH₃, and X₁, X₄, X₅, and X₆ are CH. In some embodiments, X₂ and X₇ are N and X₁, X₃, X₄, X₅, and X₆ are CR₁. In some embodiments, X₂ and X₇ are N and X₁, X₃, X₄, X₅, and X₆ are each independently CH, C (halogen), or CCH₃. In some embodiments, the halogen is F, Cl, Br, or I. In some embodiments, the halogen is F.

In some embodiments, X₂, X₃, X₄, X₈, and X₉ are each independently CR₁ or N. In some embodiments, X₂, X₃, X₄, X₈, and X₉ are CR₁. In some embodiments, X₂, X₃, X₄, X₈, and X₉ are each independently CH, C (halogen), or CCH₃. In some embodiments, one of X₂, X₃, X₄, X₈, and X₉ is N and the rest are CR₁. In some embodiments, one of X₂, X₃, X₄, X₈, and X₉ is N and the rest are each independently CH, C (halogen), or CCH₃. In some embodiments, two of X₂, X₃, X₄, X₈, and X₉ are N and the rest are CR₁. In some embodiments, two of X₂, X₃, X₄, X₈, and X₉ are N and the rest are each independently CH, C (halogen), or CCH₃. In some embodiments, three of X₂, X₃, X₄, X₈, and X₉ are N and the rest are CR₁. In some embodiments, three of X₂, X₃, X₄, X₈, and X₉ are N and the rest are each independently CH, C (halogen), or CCH₃. In some embodiments, four of X₂, X₃, X₄, X₈, and X₉ are N and one is CR₁. In some embodiments, four of X₂, X₃, X₄, X₈, and X₉ are N and one is CH, C (halogen), or CCH₃. In some embodiments, the halogen is F, Cl, Br, or I. In some embodiments, the halogen is F.

In some embodiments, the structural moiety

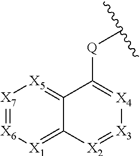

has the structure of

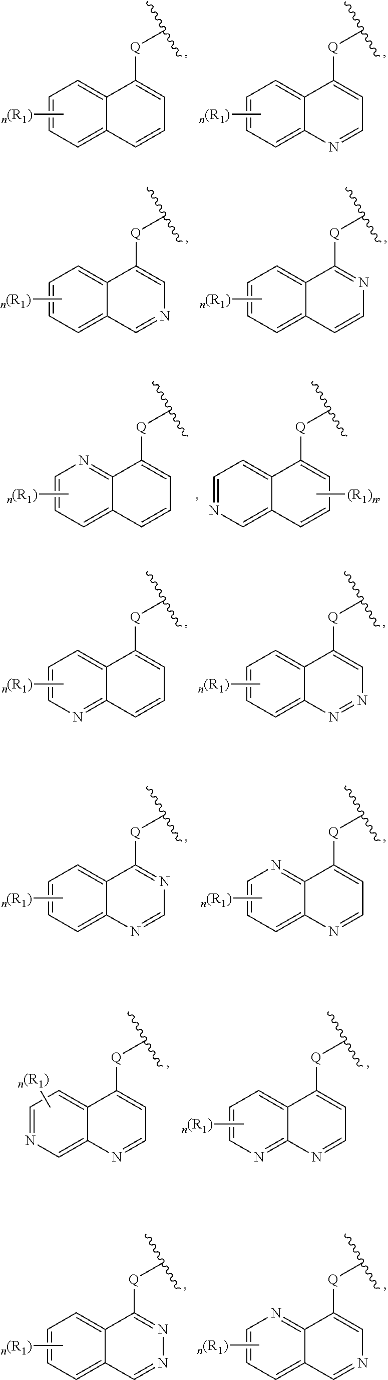

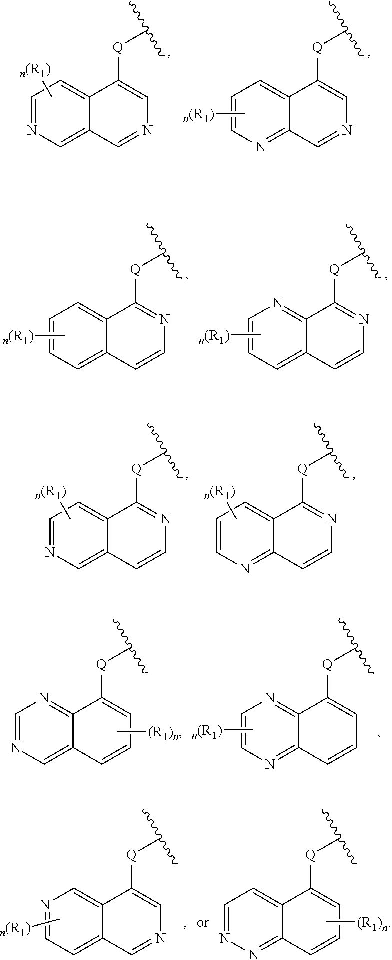

In some embodiments, the structural moiety

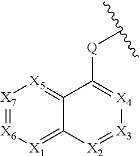

has the structure of

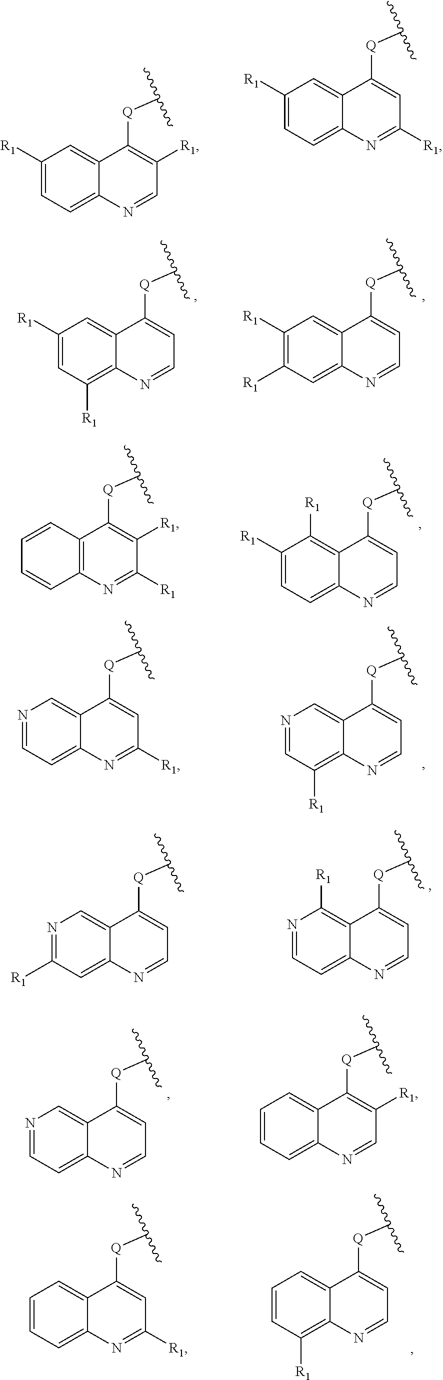

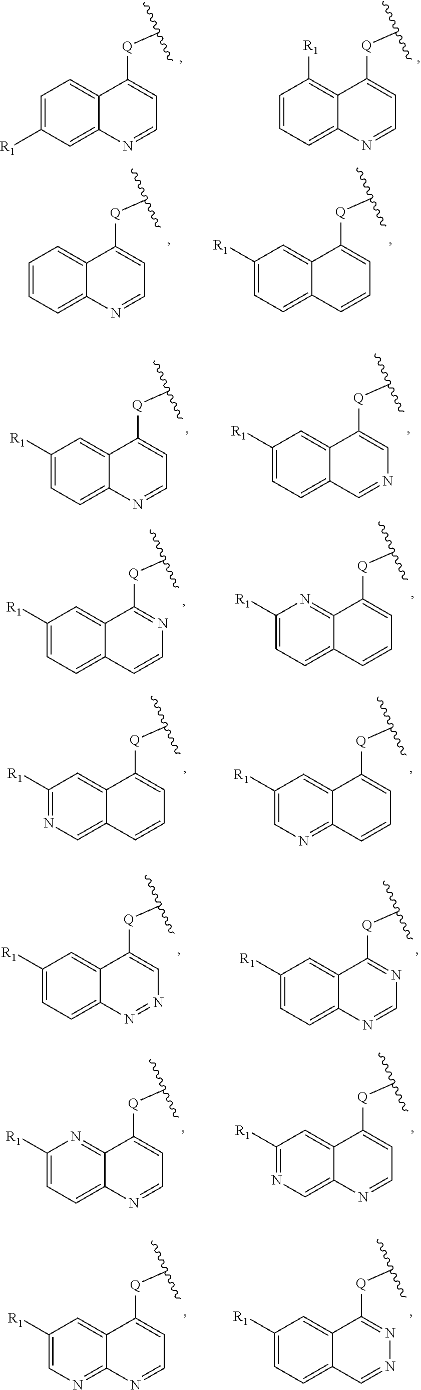

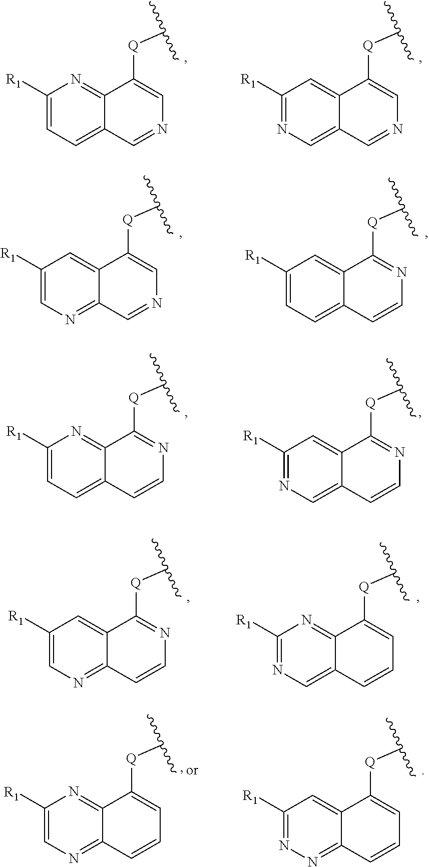

In some embodiments, Q is O. In some embodiments, Q is NRₐ, N(C═O)Rₐ, or NSO₂Rₐ. In some embodiments, Q is NH. In some embodiments, Q is NCH₃ or NCH₂CH₃.

In some embodiments, the structural moiety

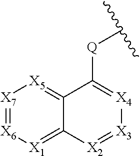

has the structure of

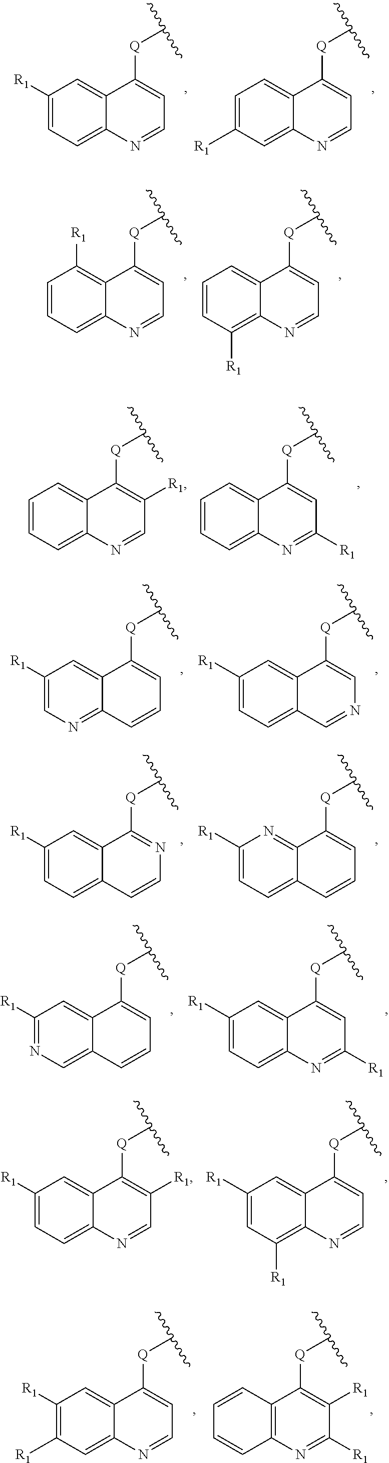

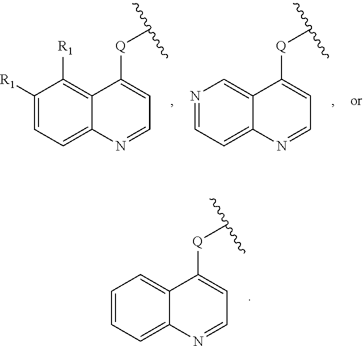

In some embodiments, the structural moiety

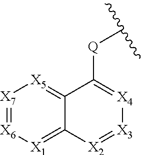

has the structure of

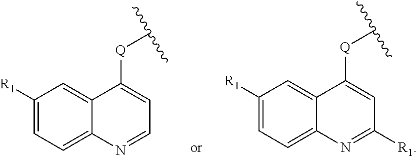

In some embodiments, Q is O. In some embodiments, Q is NRₐ, N(C═O)Rₐ, or NSO₂Rₐ. In some embodiments, Q is NH. In some embodiments, Q is NCH₃ or NCH₂CH₃.

In some embodiments, the structural moiety

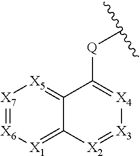

has the structure of

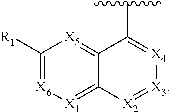

In some embodiments, the structural moiety

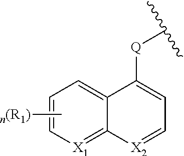

has the structure of

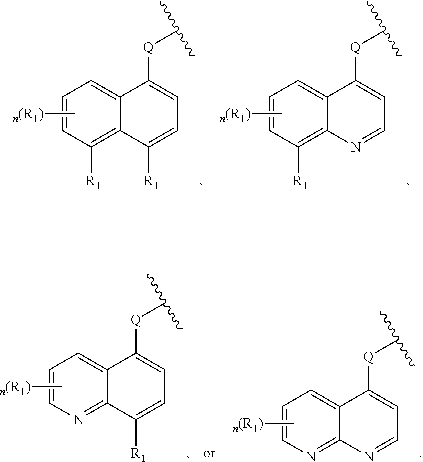

In some embodiments, the structural moiety

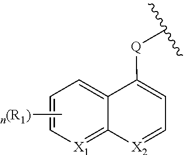

has the structure of

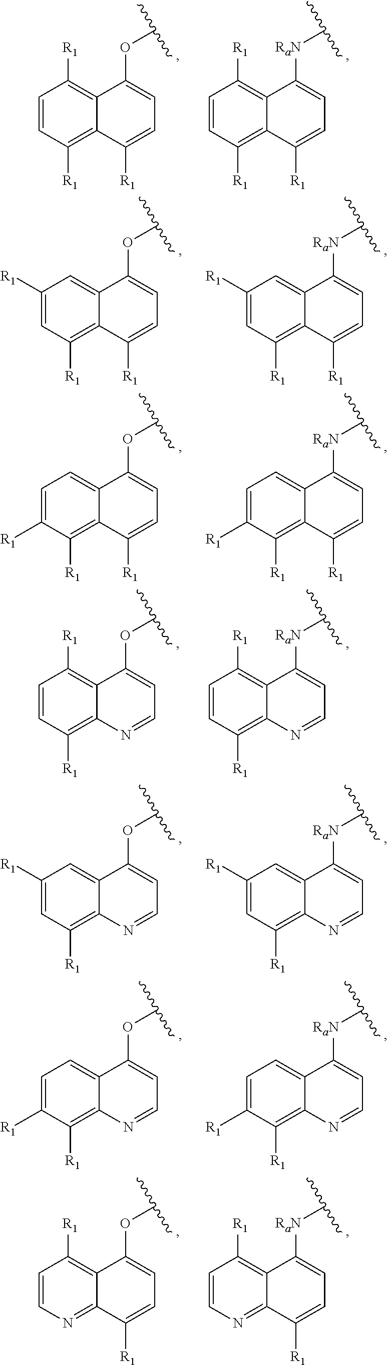

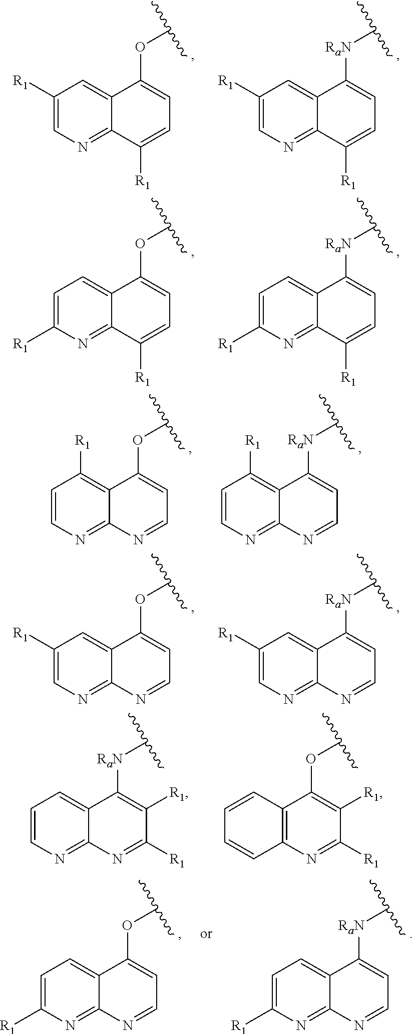

In some embodiments, the structural moiety

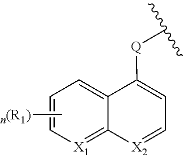

has the structure of

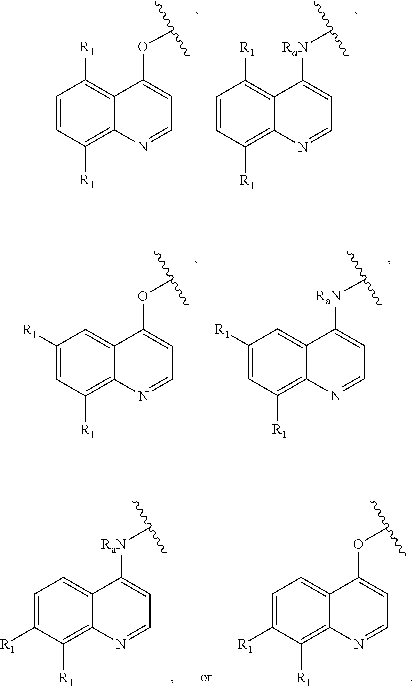

In some embodiments, the structural moiety

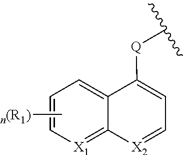

has the structure of

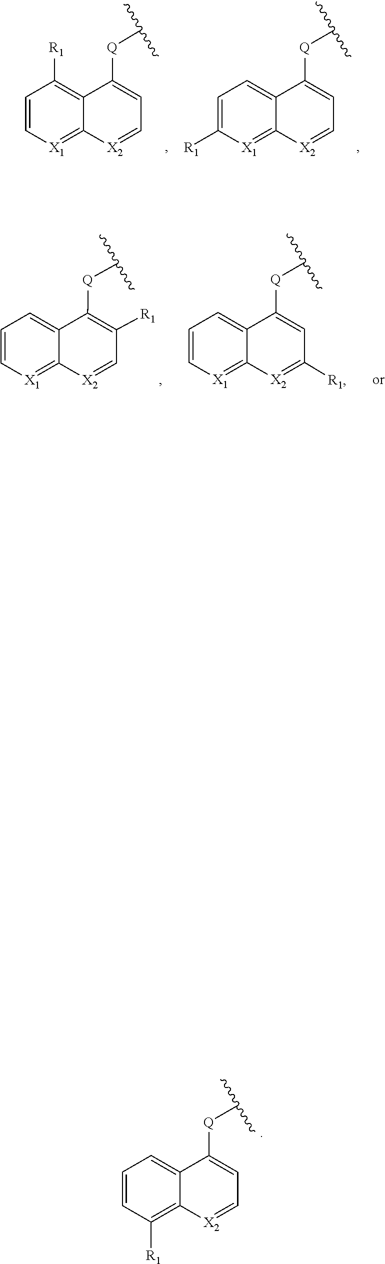

In some embodiments, the structural moiety

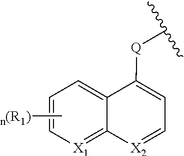

has the structure of

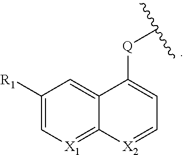

In some embodiments, the structural moiety

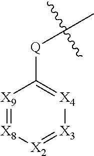

has the structure of

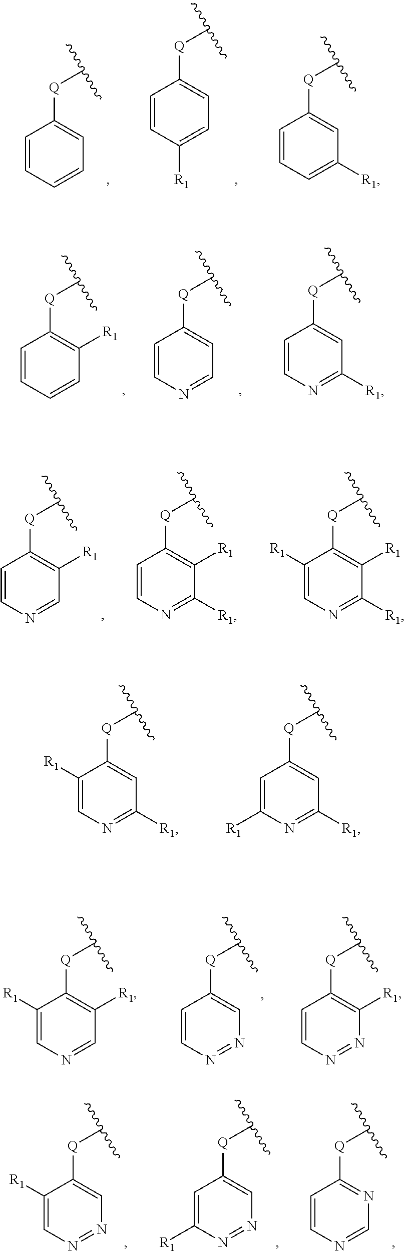

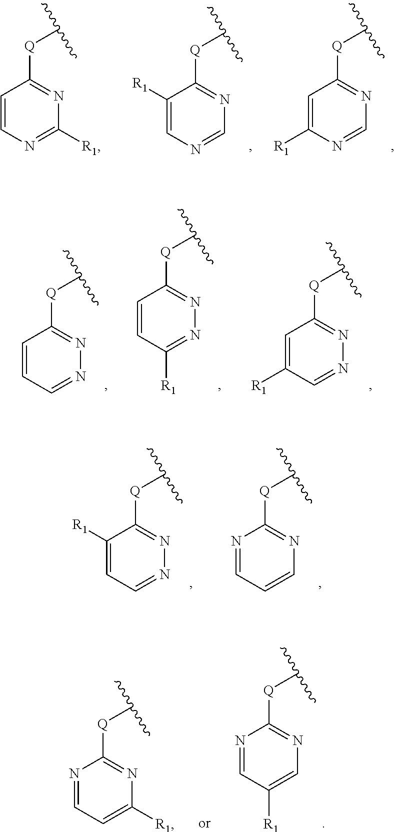

In some embodiments, the structural moiety

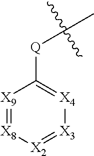

has the structure of

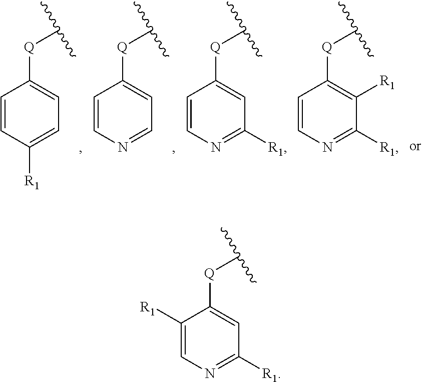

In some embodiments, the structural moiety

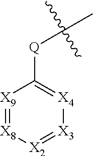

has the structure of

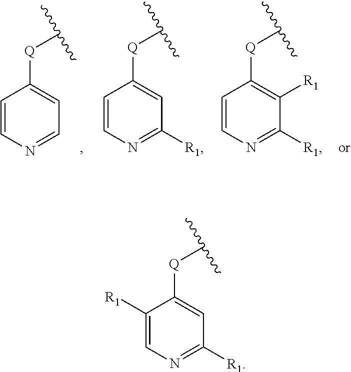

In some embodiments, Q is O. In some embodiments, Q is NRₐ, N(C—O)Rₐ, or NSO₂Rₐ. In some embodiments, Q is NH. In some embodiments, Q is NCH₃ or NCH₂CH₃.

In some embodiments, each occurrence of R₁ is independently selected from the group consisting of H, D, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₇)cycloalkyl, (C₄-C₁₀)bicycloalkyl, (C₄-C₁₄)tricycloalkyl, (C₃-C₇)heterocycloalkyl, halogenated (C₃-C₇)heterocycloalkyl, (C₄-C₁₀)heterobicycloalkyl, (C₄-C₁₄)heterotricycloalkyl, (C₄-C₁₀)heterospiroalkyl, (C₃-C₇)cycloalkenyl, (C₃-C₇)heterocycloalkenyl, (C₄-C₁₀)bicycloalkenyl, (C₄-C₁₀)heterobicycloalkenyl, (C₄-C₁₄)tricycloalkenyl, (C₄-C₁₄)heterotricycloalkenyl, aryl, heteroaryl, —ORₐ, —N(Rₐ)₂, —CORₐ, —CO₂Rₐ, CON(Rₐ)₂, —CN, —NC, NO₂, N₃, —SO₂Rₐ, —SO₂N(Rₐ)₂, and —N(Rₐ)SO₂Rₐ; wherein (C₃-C₇)cycloalkyl, (C₄-C₁₀)bicycloalkyl, (C₄-C₁₄)tricycloalkyl, (C₃-C₇)heterocycloalkyl, halogenated (C₃-C₇)heterocycloalkyl, (C₄-C₁₀)heterobicycloalkyl, (C₄-C₁₄)heterotricycloalkyl, (C₄-C₁₀)heterospiroalkyl, (C₃-C₇)cycloalkenyl, (C₃-C₇)heterocycloalkenyl, (C₄-C₁₀)bicycloalkenyl, (C₄-C₁₀)heterobicycloalkenyl, (C₄-C₁₄)tricycloalkenyl, (C₄-C₁₄)heterotricycloalkenyl, aryl, and heteroaryl are each optionally substituted with one or more (C₁-C₆)alkyl. In some embodiments, each occurrence of R₁ is independently selected from the group consisting of (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₇)cycloalkyl, (C₄-C₁₀)bicycloalkyl, (C₃-C₇)heterocycloalkyl, and (C₄-C₁₀)heterobicycloalkyl; wherein the (C₃-C₇)cycloalkyl, (C₄-C₁₀)bicycloalkyl, (C₃-C₇)heterocycloalkyl, and (C₄-C₁₀)heterobicycloalkyl are each optionally substituted with one or more (C₁-C₆)alkyl. In some embodiments, each occurrence of R₁ is independently selected from the group consisting of aryl and heteroaryl; wherein the (C₄-C₁₀)heterospiroalkyl, aryl, and heteroaryl are each optionally substituted with one or more (C₁-C₆)alkyl. In some embodiments, each occurrence of R₁ is independently optionally substituted (C₃-C₇)cycloalkenyl or optionally substituted (C₃-C₇)heterocycloalkenyl. In some embodiments, each occurrence of R₁ is independently optionally substituted (C₄-C₁₀)bicycloalkenyl or optionally substituted (C₄-C₁₀)heterobicycloalkenyl. In some embodiments, each occurrence of R₁ is independently optionally substituted (C₄-C₁₄)tricycloalkenyl or optionally substituted (C₄-C₁₄)heterotricycloalkenyl. In some embodiments, each occurrence of R₁ is independently optionally substituted (C₄-C₁₄)tricycloalkyl or optionally substituted (C₄-C₁₄)heterotricycloalkyl. In some embodiments, each occurrence of R₁ is independently selected from the group consisting of —ORₐ, —SRₐ, —N(Rₐ)₂, —CORₐ, —CO₂Rₐ, CON(Rₐ)₂, —CN, —NC, NO₂, N₃, —SO₂Rₐ, —SO₂N(Rₐ)₂, and —N(Rₐ)SO₂Rₐ. In some embodiments, each occurrence of R₁ is independently selected from the group consisting of

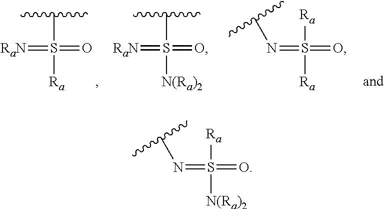

In some embodiments, each occurrence of R₁ is independently H, D, halogen, ORₐ, N(Rₐ)₂, (C₁-C₆)alkyl, (C₃-C₇)heterocycloalkyl, (C₄-C₁₀)heterospiroalkyl, halogenated (C₃-C₇)heterocycloalkyl, (C₁-C₆)alkynyl, aryl, (C₄-C₁₀)bicycloalkyl, —CN, —NC, N₃, NO₂, CORₐ, CO₂Rₐ, CON(Rₐ)₂, —SO₂Rₐ, or —SO₂N(Rₐ)₂; wherein the (C₃-C₇)heterocycloalkyl, (C₄-C₁₀)heterospiroalkyl, aryl, and (C₄-C₁₀)bicycloalkyl are each optionally substituted with one or more (C₁-C₆)alkyl. In some embodiments, each occurrence of R₁ is independently H, D, halogen, (C₁-C₆)alkyl, (C₃-C₇)heterocycloalkyl, (C₄-C₁₀)heterospiroalkyl, halogenated (C₃-C₇)heterocycloalkyl, N(Rₐ)₂, or —CN; wherein the (C₃-C₇)heterocycloalkyl and (C₄-C₁₀)heterospiroalkyl are each optionally substituted with one or more (C₁-C₆)alkyl. In some embodiments, at least one occurrence of R₁ is (C₄-C₁₀)heterospiroalkyl. In some embodiments, at least one occurrence of R₁ is halogenated (C₃-C₇)heterocycloalkyl. In some embodiments, each occurrence of R₁ is independently H, (C₁-C₆)alkyl, (C₁-C₆)alkynyl, aryl, (C₄-C₁₀)bicycloalkyl, —SO₂Rₐ, or —SO₂N(Rₐ)₂; wherein the aryl and (C₄-C₁₀)bicycloalkyl are each optionally substituted with one or more (C₁-C₆)alkyl. In some embodiments, at least one occurrence of R₁ is (C₄-C₁₀)heterospiroalkyl, optionally substituted with one or more (C₁-C₆)alkyl. In some embodiments, at least one occurrence of R₁ is halogenated (C₃-C₇)heterocycloalkyl, optionally substituted with one or more (C₁-C₆)alkyl. In some embodiments, each occurrence of R₁ is independently H, D, F, Cl, Br, CH₃, OCH₃, NH₂, NHCH₃, N(CH₃)₂,

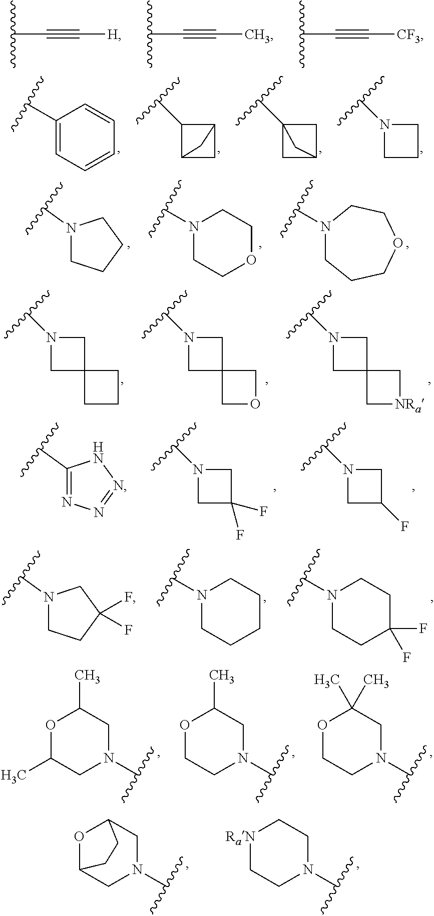

—CN, —NC, N₃, NO₂,

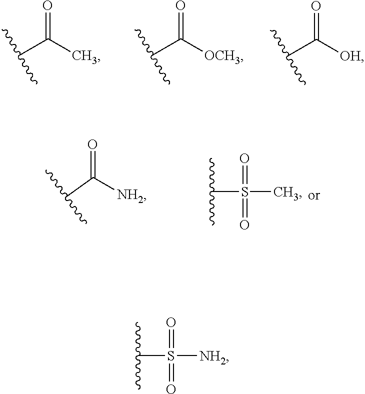

where Rₐ′ is H or (C₁-C₆)alkyl. In some embodiments, each occurrence of R₁ is independently H, D, F, CH₃, N(CH₃)₂,

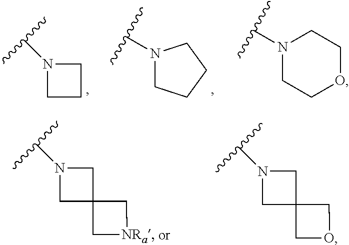

where Rₐ′ is H or (C₁-C₆)alkyl. In some embodiments, each occurrence of R₁ is independently

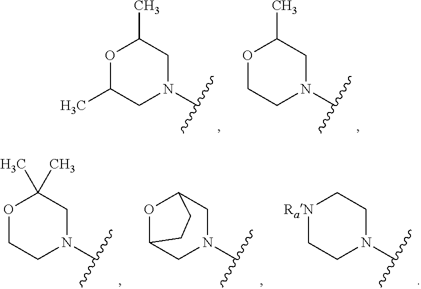

In some embodiments, the (C₃-C₇)cycloalkyl, (C₄-C₁₀)bicycloalkyl, (C₃-C₇)heterocycloalkyl, (C₄-C₁₀)heterobicycloalkyl, (C₄-C₁₀)heterospiroalkyl, (C₃-C₇)cycloalkenyl, (C₃-C₇)heterocycloalkenyl, (C₄-C₁₀)bicycloalkenyl, (C₄-C₁₀)heterobicycloalkenyl, (C₄-C₁₄)tricycloalkenyl, (C₄-C₁₄)heterotricycloalkenyl, (C₄-C₁₄)tricycloalkyl, (C₄-C₁₄)heterotricycloalkyl, aryl, and heteroaryl of R₁ are each optionally substituted by one or more halogen, (C₁-C₆)alkyl, —ORₐ, —CN, or —N(Rₐ)₂.

In some embodiments, at least one occurrence of R₁ is a partially saturated bicyclic heteroaryl optionally substituted by one or more (C₁-C₆)alkyl, halogenated (C₁-C₆)alkyl, —SO₂Rₐ, or —SO₂N(Rₐ)₂. In some embodiments, at least one occurrence of R₁ is

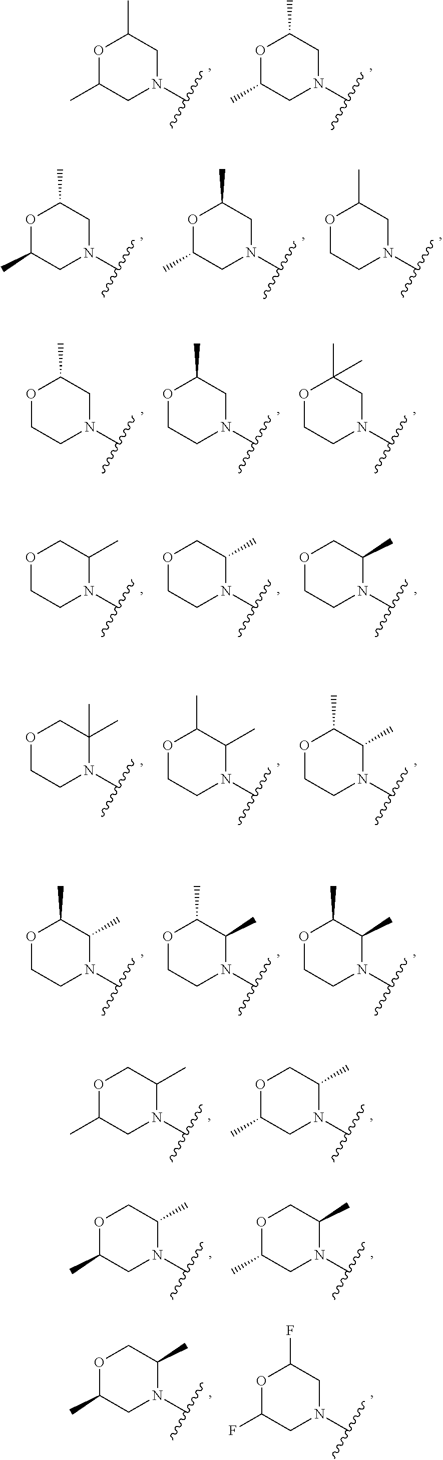

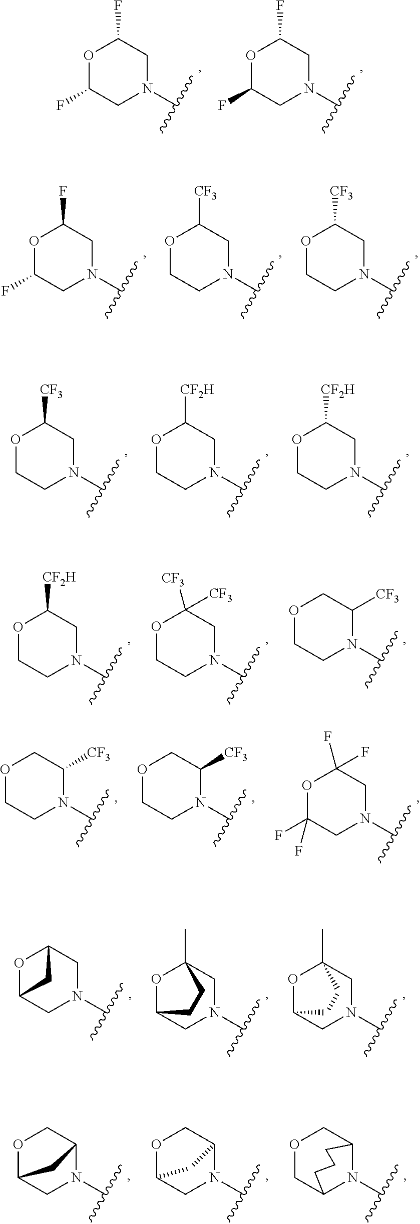

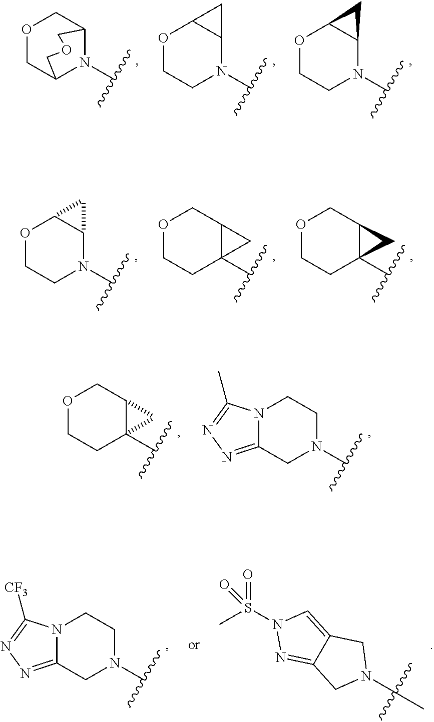

In some embodiments, \textcircled{A} is

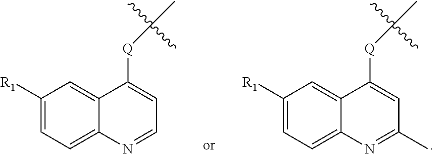

In some embodiments, at least one occurrence of R₁ is H, D, or halogen. In some embodiments, at least one occurrence of R₁ is H. In some embodiments, at least one occurrence of R₁ is D. In some embodiments, at least one occurrence of R₁ is F. In some embodiments, at least one occurrence of R₁ is CH₃. In some embodiments, at least one occurrence of R₁ is OCH₃. In some embodiments, at least one occurrence of R₁ is NH₂. In some embodiments, at least one occurrence of R₁ is NHCH₃. In some embodiments, at least one occurrence of R₁ is N(CH₃)₂. In some embodiments, at least one occurrence of R₁ is F. In some embodiments, at least one occurrence of R₁ is

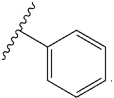 [SMILES: *c1ccccc1]

In some embodiments, at least one occurrence of R₁ is

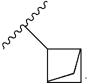 [SMILES: *C1CCC1]

In some embodiments, at least one occurrence of R₁ is

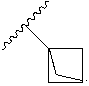 [SMILES: *C1CCC1]

In some embodiments, at least one occurrence of R₁ is

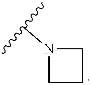 [SMILES: *N1CCC1]

In some embodiments, at least one occurrence of R₁ is

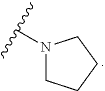 [SMILES: *N1CCCC1]

In some embodiments, at least one occurrence of R₁ is

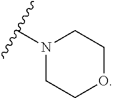 [SMILES: *N1CCOCC1]

In some embodiments, at least one occurrence of R₁ is

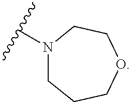 [SMILES: *N1CCCOCC1]

In some embodiments, at least one occurrence of R₁ is

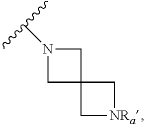

where Rₐ′ is H or (C₁-C₆)alkyl. In some embodiments, at least one occurrence of R₁ is

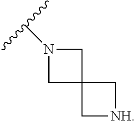 [SMILES: *N1CC2(CNC2)C1]

In some embodiments, at least one occurrence of R₁ is

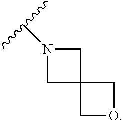 [SMILES: *N1CC2(COC2)C1]

In some embodiments, at least one occurrence of R₁ is

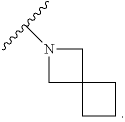 [SMILES: *N1CC2(CCC2)C1]

In some embodiments, at least one occurrence of R₁ is

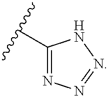 [SMILES: *c1nnn[nH]1]

In some embodiments, at least one occurrence of R₁ is

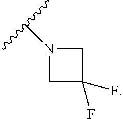 [SMILES: *N1CC(F)(F)C1]

In some embodiments, at least one occurrence of R₁ is

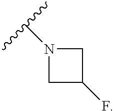 [SMILES: *N1CC(F)C1]

In some embodiments, at least one occurrence of R₁ is

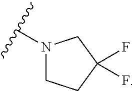 [SMILES: *N1CCC(F)(F)C1]

In some embodiments, at least one occurrence of R₁ is

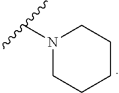 [SMILES: *N1CCCCC1]

In some embodiments, at least one occurrence of R₁ is

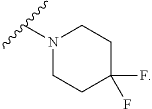 [SMILES: *N1CCC(F)(F)CC1]

In some embodiments, at least one occurrence of R₁ is

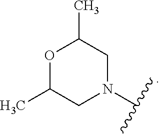 [SMILES: *N1CC(C)OC(C)C1]

In some embodiments, at least one occurrence of R₁ is

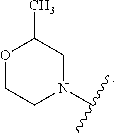 [SMILES: *N1CCOC(C)C1]

In some embodiments, at least one occurrence of R₁ is

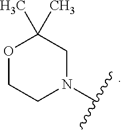 [SMILES: *N1CCOC(C)(C)C1]

In some embodiments, at least one occurrence of R₁ is

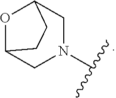 [SMILES: *N1CC2CCC(C1)O2]

In some embodiments, at least one occurrence of R₁ is

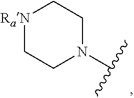

wherein Rₐ′ is H or (C₁-C₆)alkyl. In some embodiments, at least one occurrence of R₁ is

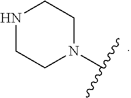 [SMILES: *N1CCNCC1]

In some embodiments, at least one occurrence of R₁ is

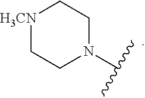

In some embodiments, at least one occurrence of R₁ is

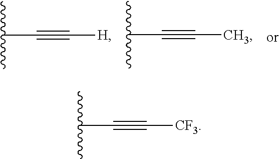

In some embodiments, at least one occurrence of R₁ is

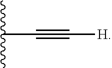

In some embodiments, at least one occurrence of R₁ is

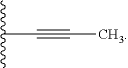 [SMILES: *C#CC]

In some embodiments, at least one occurrence of R₁ is

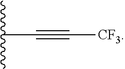 [SMILES: *C#CC(F)(F)F]

In some embodiments, at least one occurrence of R₁ is —CN. In some embodiments, at least one occurrence of R₁ is —NC. In some embodiments, at least one occurrence of R₁ is

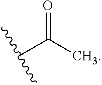 [SMILES: *C(C)=O]

In some embodiments, at least one occurrence of R₁ is

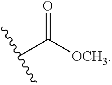 [SMILES: *C(=O)OC]

In some embodiments, at least one occurrence of R₁ is

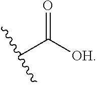 [SMILES: *C(=O)O]

In some embodiments, at least one occurrence of R₁ is

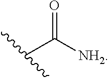 [SMILES: *C(N)=O]

In some embodiments, at least one occurrence of R₁ is NO₂. In some embodiments, at least one occurrence of R₁ is N₃. In some embodiments, at least one occurrence of R₁ is

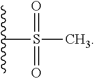 [SMILES: *S(C)(=O)=O]

In some embodiments, at least one occurrence of R₁ is

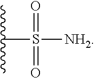 [SMILES: *S(N)(=O)=O]

In some embodiments, at least one occurrence of R₁ is

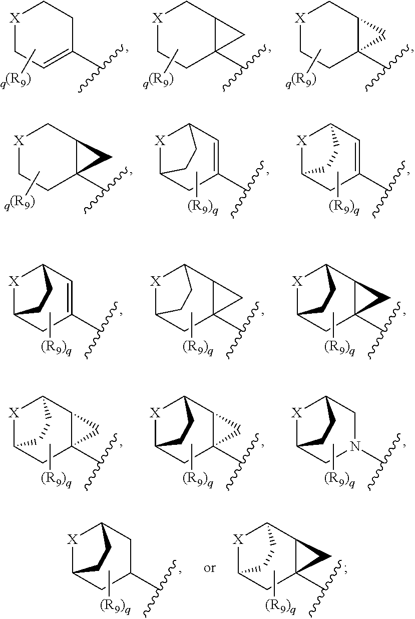

wherein X is CR₁₅, O, NR₁₄, or S; each occurrence of R₉ is independently H, (C₁-C₆)alkyl, halogenated (C₁-C₆)alkyl, halogen, —ORₐ, —CN, or —N(Rₐ)₂; R₁₄ is H, (C₁-C₆)alkyl, (C₃-C₇)cycloalkyl, (C₃-C₇)heterocycloalkyl, aryl, or heteroaryl; each occurrence of R₁₅ is independently H, (C₁-C₆)alkyl, halogenated (C₁-C₆)alkyl, halogen, —ORₐ, —CN, or —N(Rₐ)₂; and q is 0, 1, 2, or 3.

In some embodiments, X is O. In some embodiments, X is S. In some embodiments, X is CR₁₅. In some embodiments, X is NR₁₅.

In some embodiments, each occurrence of R₉ is independently H, (C₁-C₆)alkyl, halogenated (C₁-C₆)alkyl, halogen, —ORₐ, —CN, or —N(Rₐ)₂. In some embodiments, each occurrence of R₉ is independently H, (C₁-C₆)alkyl, halogenated (C₁-C₆)alkyl, or halogen. In some embodiments, each occurrence of R₉ is independently H, F, Cl, Br, CH₃, CF₃, OH, NH₂, —NHCH₃, or —N(CH₃)₂. In some embodiments, each occurrence of R₉ is independently H, F, Cl, Br or CH₃.

In some embodiments, each occurrence of R₁₅ is independently H, (C₁-C₆)alkyl, halogenated (C₁-C₆)alkyl, halogen, —ORₐ, —CN, or —N(Rₐ)₂. In some embodiments, each occurrence of R₁₅ is independently H, (C₁-C₆)alkyl, halogenated (C₁-C₆)alkyl, or halogen. In some embodiments, each occurrence of R₁₅ is independently H, F, Cl, Br, CH₃, CF₃, OH, NH₂, —NHCH₃, or —N(CH₃)₂. In some embodiments, each occurrence of R₁₅ is independently H, F, Cl, Br or CH₃.

In some embodiments, q is 0. In some embodiments, q is 1. In some embodiments, q is 2. In some embodiments, q is 3.

In some embodiments, R₁₄ is H, (C₁-C₆)alkyl, (C₃-C₇)cycloalkyl, (C₃-C₇)heterocycloalkyl, aryl, or heteroaryl. In some embodiments, R₁₄ is H, (C₁-C₆)alkyl or (C₃-C₇)cycloalkyl. In some embodiments, R₁₄ is H or (C₁-C₆)alkyl. In some embodiments, R₁₄ is H or CH₃.

In some embodiments, at least one occurrence of R₁ is

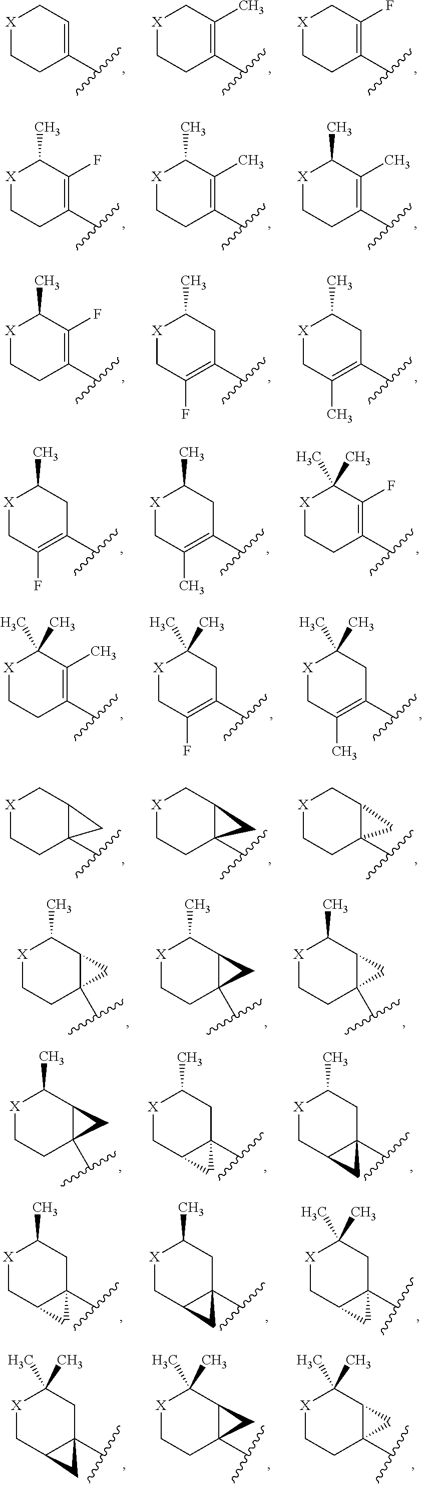

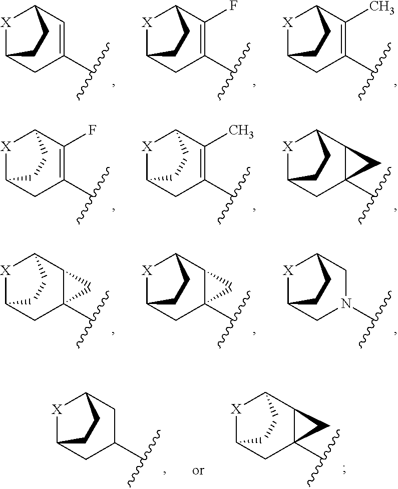

wherein X is O or NR₁₄; and R₁₄ is H or (C₁-C₆)alkyl. In some embodiments, at least one occurrence of R₁ is

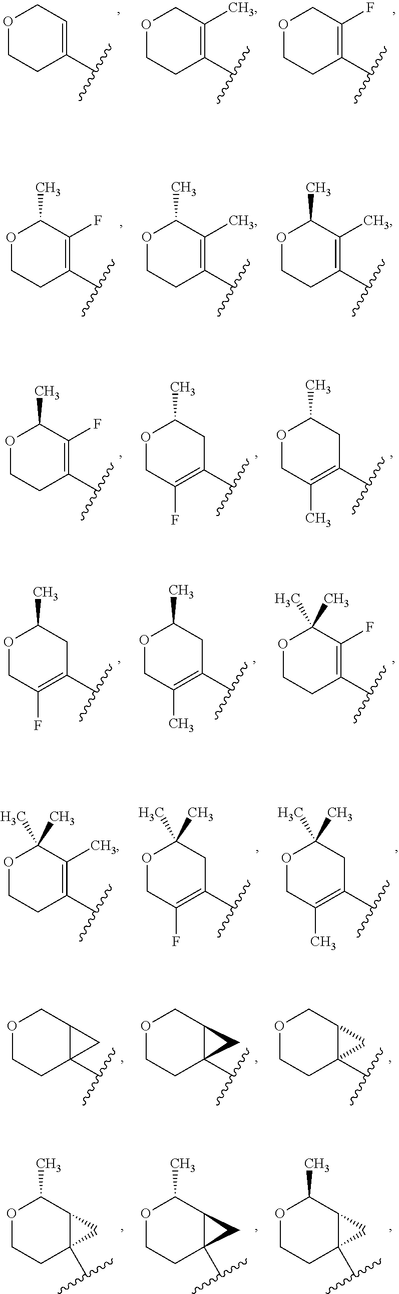

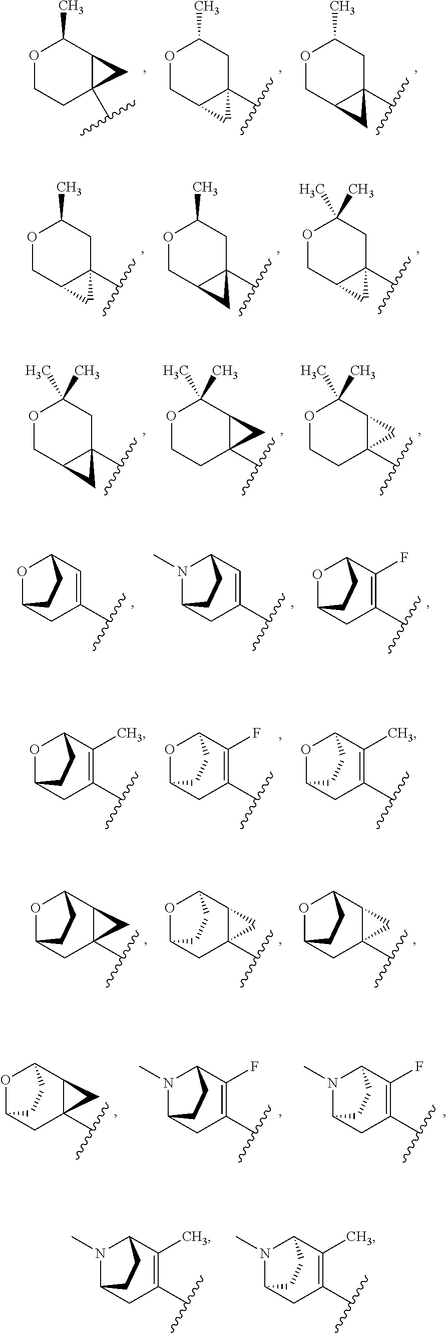

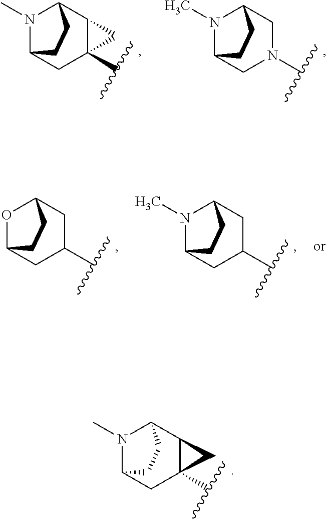

In some embodiments, \textcircled{A} is

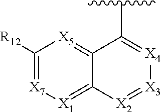

wherein R₁₂ is (C₃-C₇)cycloalkenyl, (C₃-C₇)heterocycloalkenyl, (C₄-C₁₀)bicycloalkenyl, (C₄-C₁₀)heterobicycloalkenyl, (C₄-C₁₄)tricycloalkenyl, or (C₄-C₁₄)heterotricycloalkenyl, each of which is optionally substituted by one or more (C₁-C₆)alkyl, halogenated (C₁-C₆)alkyl, halogen, —ORₐ, —CN, or —N(Rₐ)₂. In some embodiments, R₁₂ is

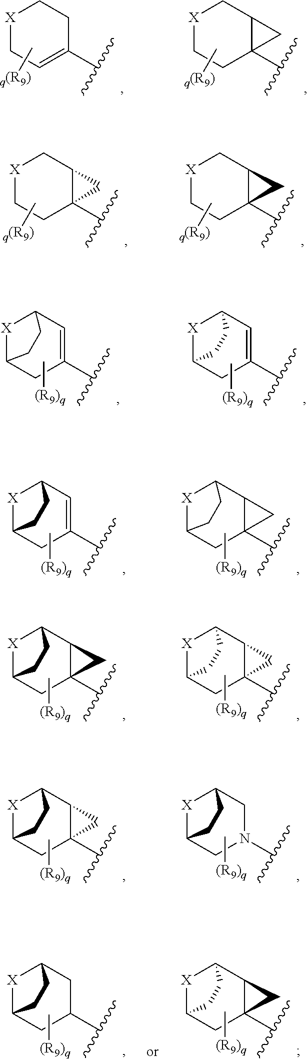

wherein X is CR₁₅, O, NR₁₄, or S; each occurrence of R₉ is independently H, (C₁-C₆)alkyl, halogenated (C₁-C₆)alkyl, halogen, —ORₐ, —CN, or —N(Rₐ)₂; R₁₄ is H, (C₁-C₆)alkyl, (C₃-C₇)cycloalkyl, (C₃-C₇)heterocycloalkyl, aryl, or heteroaryl; each occurrence of R₁₅ is independently H, (C₁-C₆)alkyl, halogenated (C₁-C₆)alkyl, halogen, —ORₐ, —CN, or —N(Rₐ)₂; and q is 0, 1, 2, or 3. In some embodiments, X₂ is N. In some embodiments, X₃ is CH or CCH₃. In some embodiments, \textcircled{A} is

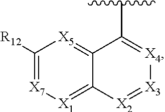

where X₂ is N; X₃ is CH or CCH₃; and R₁₂ is (C₃-C₇)cycloalkenyl, (C₃-C₇)heterocycloalkenyl, (C₄-C₁₀)bicycloalkenyl, (C₄-C₁₀)heterobicycloalkenyl, (C₄-C₁₄)tricycloalkenyl, or (C₄-C₁₄)heterotricycloalkenyl, each of which is optionally substituted by one or more (C₁-C₆)alkyl, halogenated (C₁-C₆)alkyl, halogen, —ORₐ, —CN, or —N(Rₐ)₂.

In some embodiments, X is O. In some embodiments, X is S. In some embodiments, X is CR₁₅. In some embodiments, X is NR₁₄.

In some embodiments, each occurrence of R₉ is independently H, (C₁-C₆)alkyl, halogenated (C₁-C₆)alkyl, halogen, —ORₐ, —CN, or —N(Rₐ)₂. In some embodiments, each occurrence of R₉ is independently H, (C₁-C₆)alkyl, halogenated (C₁-C₆)alkyl, or halogen. In some embodiments, each occurrence of R₉ is independently H, F, Cl, Br, CH₃, CF₃, OH, NH₂, —NHCH₃, or —N(CH₃)₂. In some embodiments, each occurrence of R₉ is independently H, F, Cl, Br or CH₃.

In some embodiments, each occurrence of R₁₅ is independently H, (C₁-C₆)alkyl, halogenated (C₁-C₆)alkyl, halogen, —ORₐ, —CN, or —N(Rₐ)₂. In some embodiments, each occurrence of R₁₅ is independently H, (C₁-C₆)alkyl, halogenated (C₁-C₆)alkyl, or halogen. In some embodiments, each occurrence of R₁₅ is independently H, F, Cl, Br, CH₃, CF₃, OH, NH₂, —NHCH₃, or —N(CH₃)₂. In some embodiments, each occurrence of R₁₅ is independently H, F, Cl, Br or CH₃.

In some embodiments, q is 0. In some embodiments, q is 1. In some embodiments, q is 2. In some embodiments, q is 3.

In some embodiments, R₁₄ is H, (C₁-C₆)alkyl, (C₃-C₇)cycloalkyl, (C₃-C₇)heterocycloalkyl, aryl, or heteroaryl. In some embodiments, R₁₄ is H, (C₁-C₆)alkyl or (C₃-C₇)cycloalkyl. In some embodiments, R₁₄ is H or (C₁-C₆)alkyl. In some embodiments, R₁₄ is H or CH₃.

In some embodiments, at least one occurrence of R₁₂ is

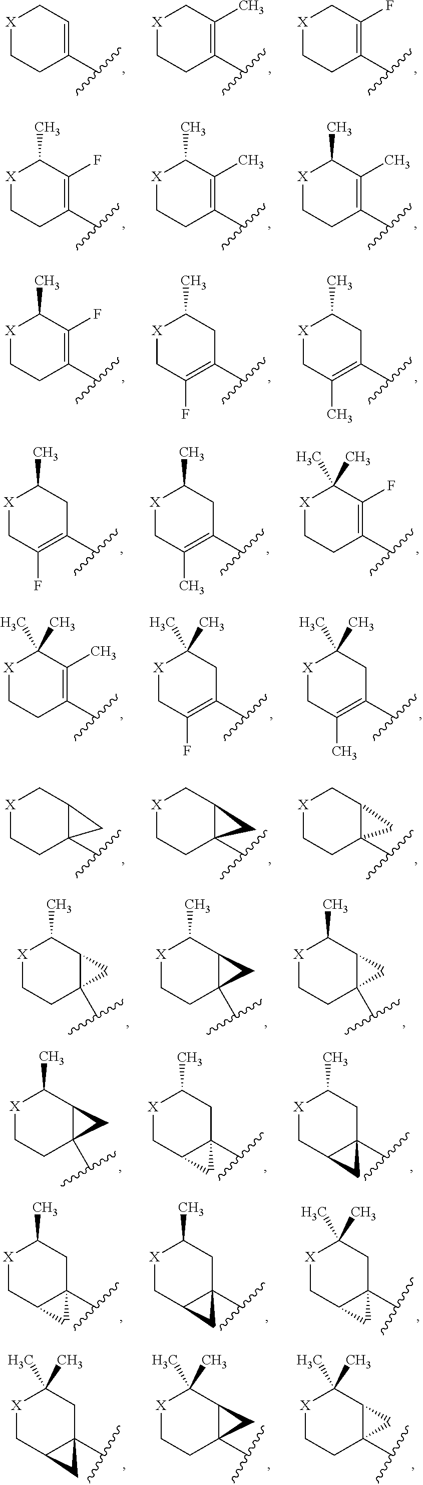

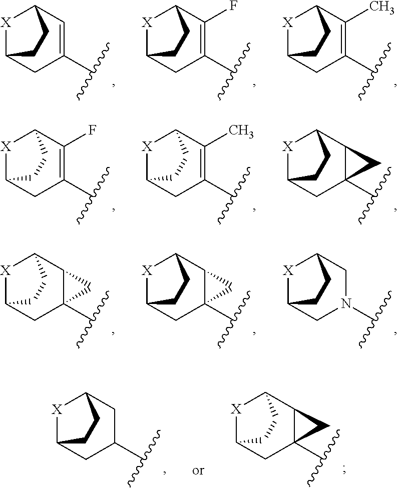

wherein X is O or NR₁₄; and R₁₄ is H or (C₁-C₆)alkyl. In some embodiments, at least one occurrence of R₁₂ is

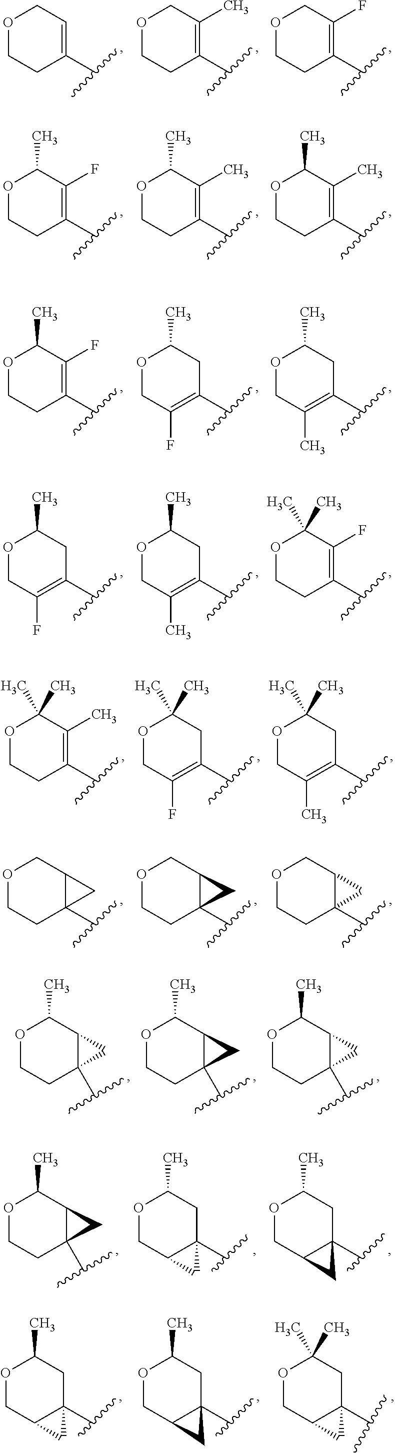

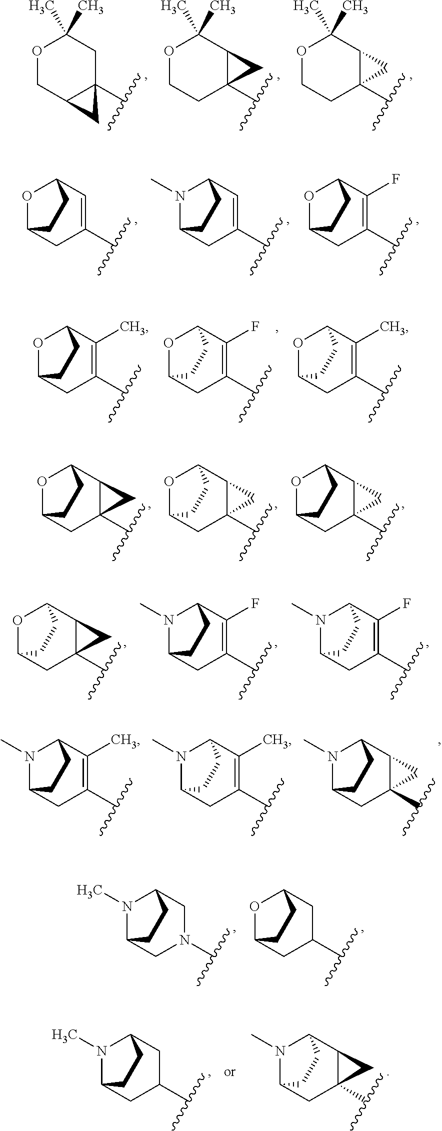

In some embodiments, the structural moiety

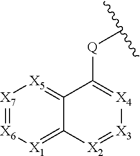

has the structure of

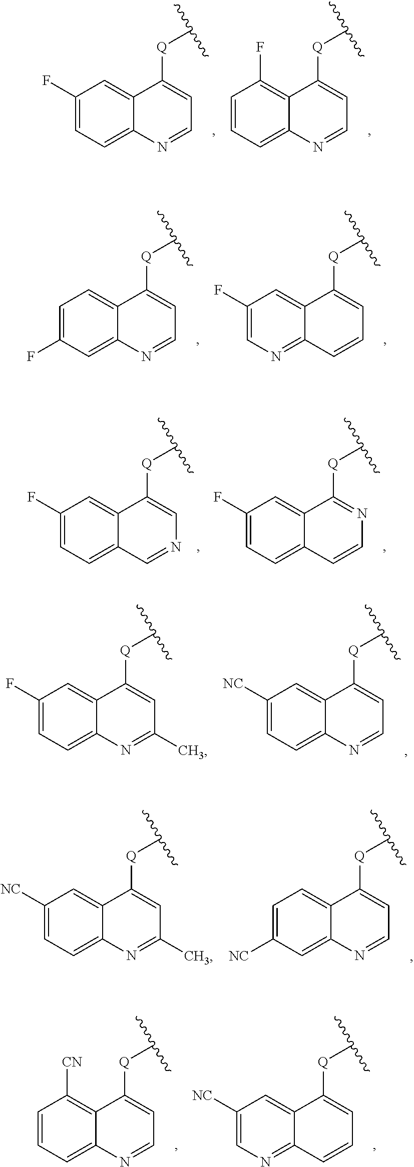

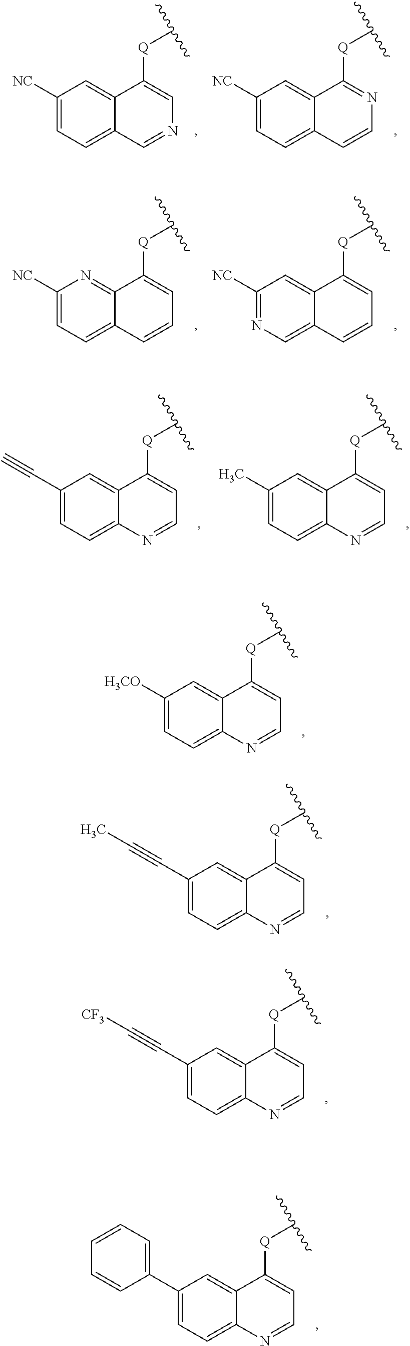

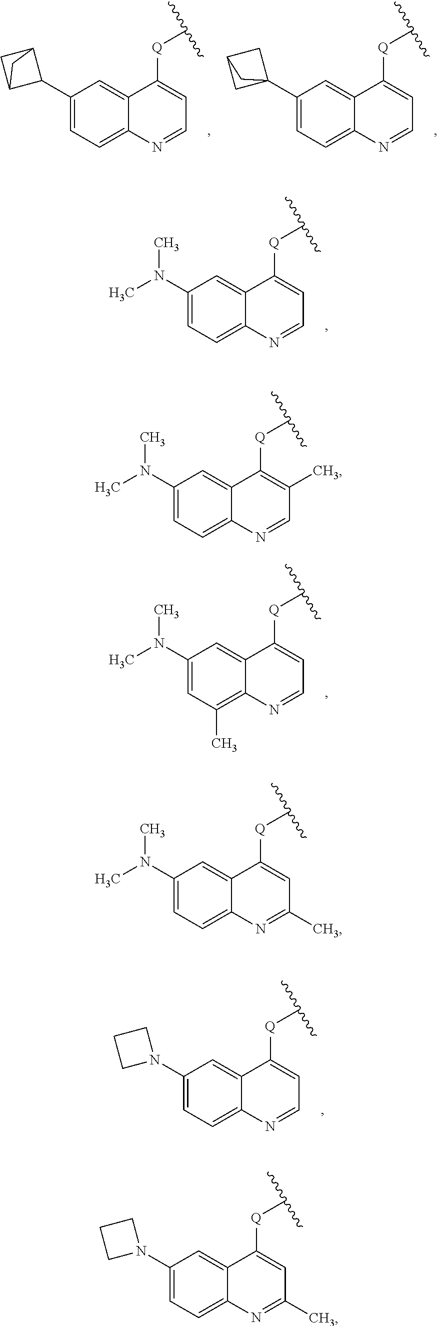

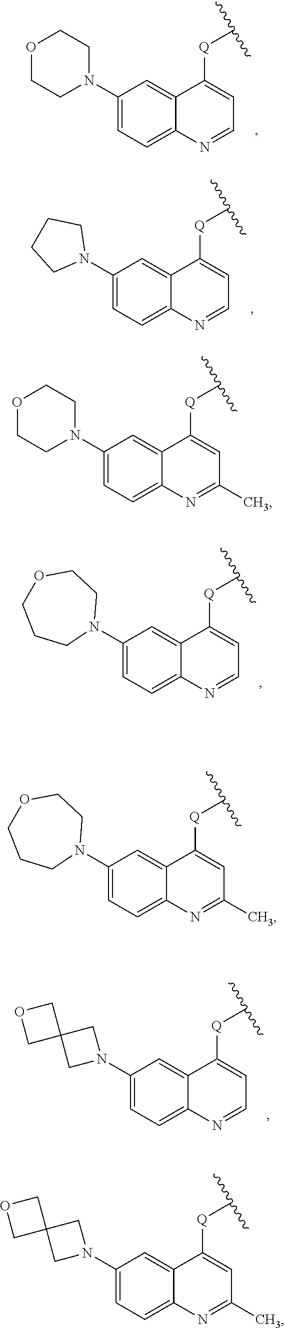

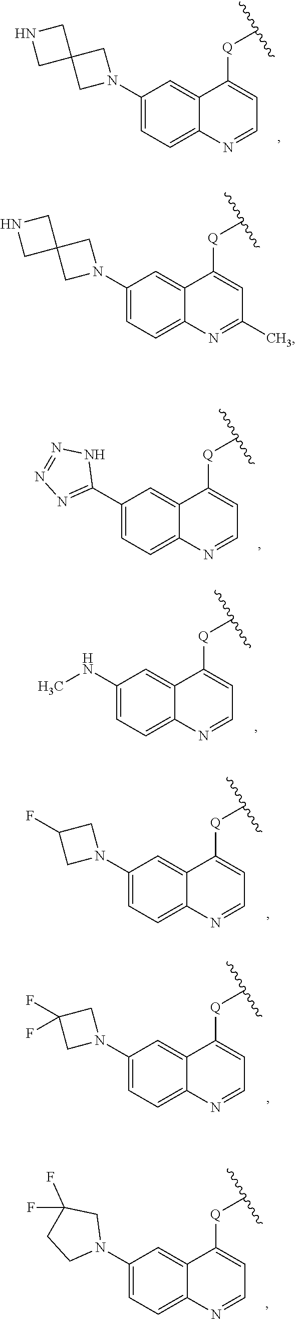

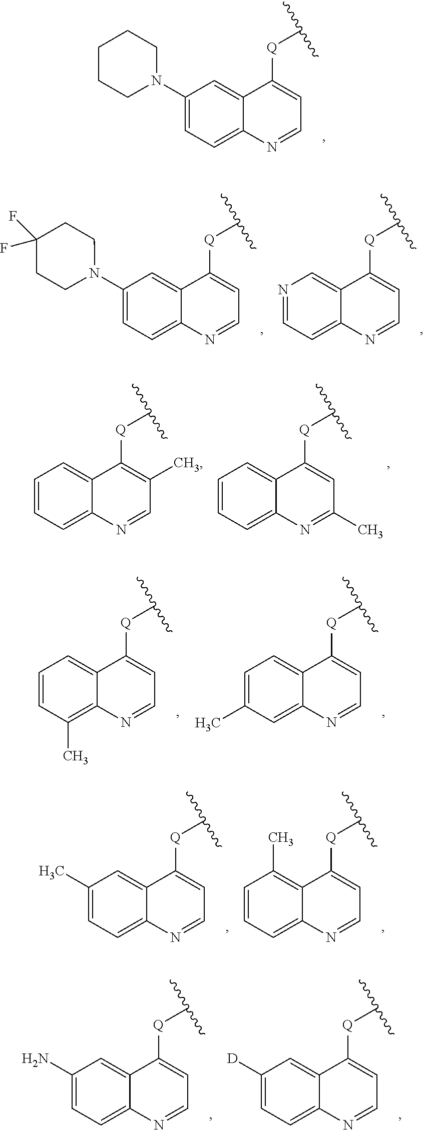

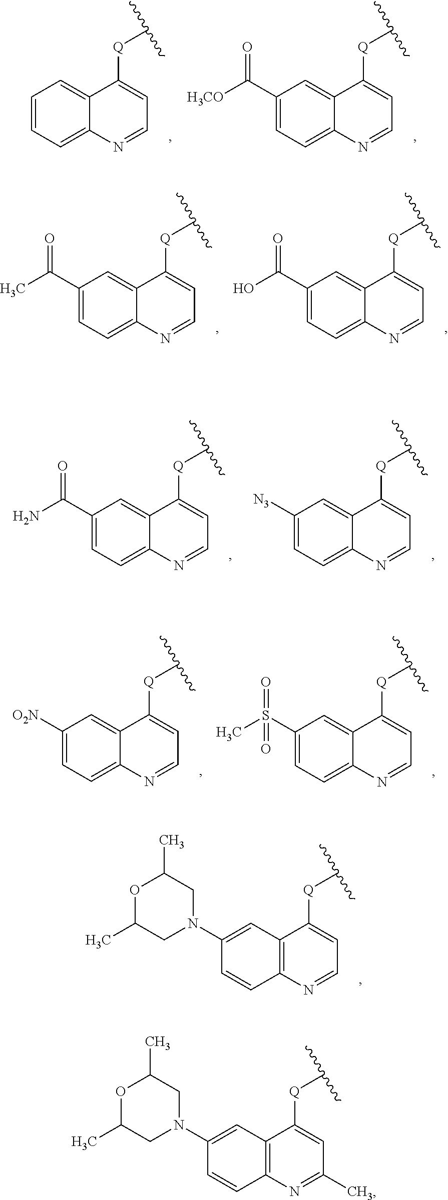

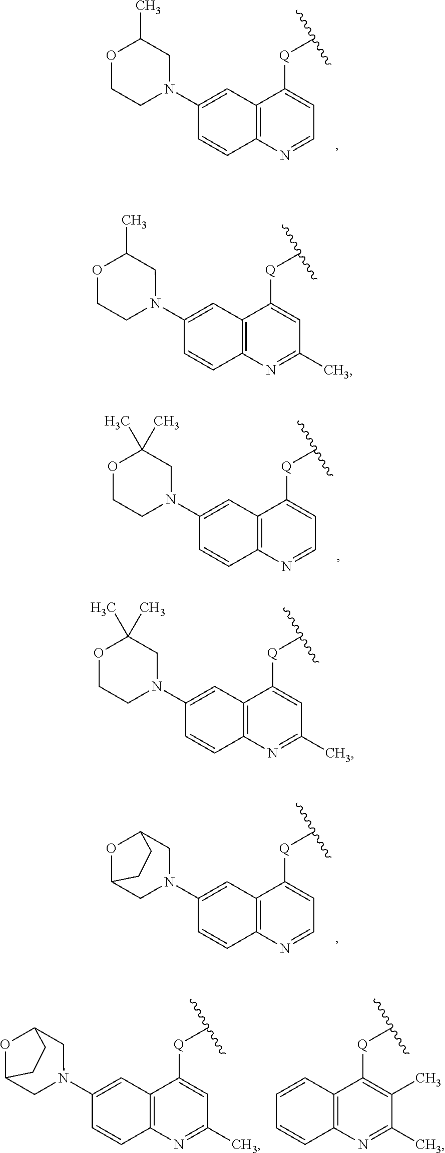

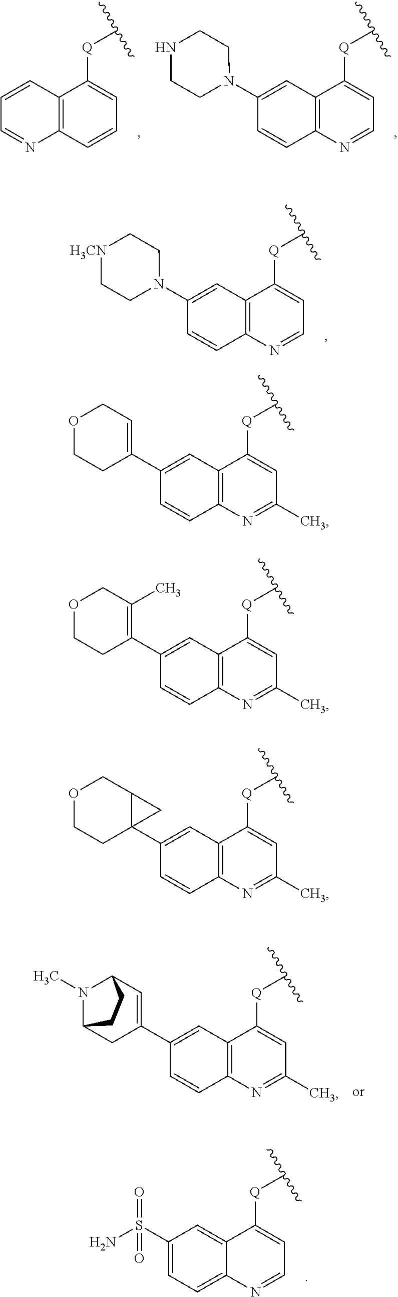

In some embodiments, the structural moiety

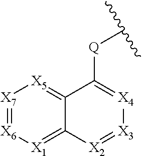

has the structure of

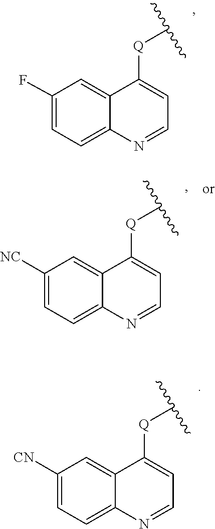

In some embodiments, the structural moiety

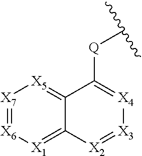

has the structure of

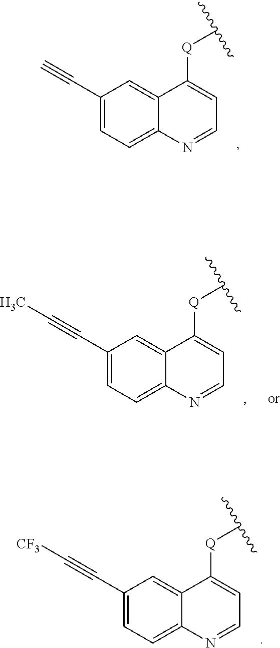

In some embodiments, the structural moiety

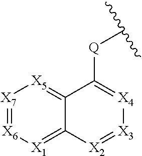

has the structure of

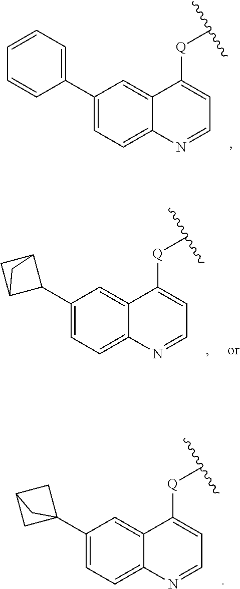

In some embodiments, the structural moiety

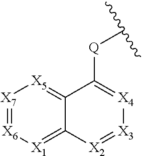

has the structure of

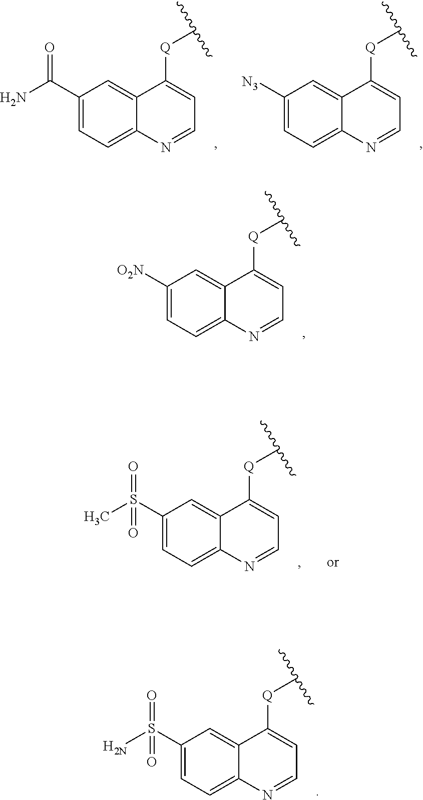

In some embodiments, the structural moiety

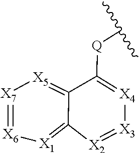

has the structure of

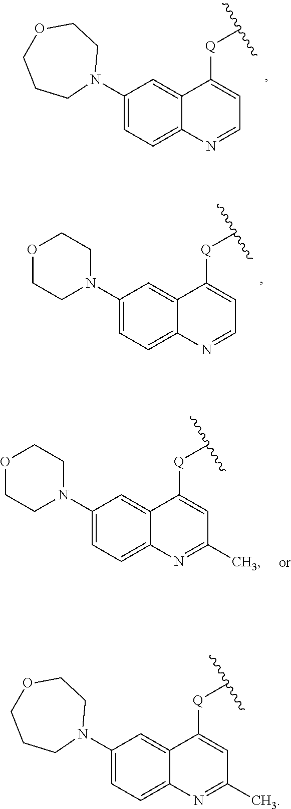

In some embodiments, the structural moiety

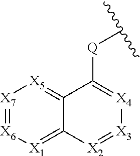

has the structure of

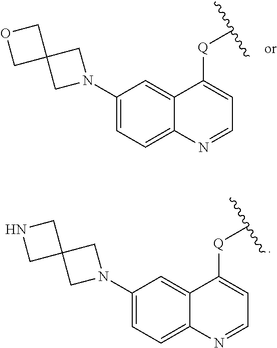

In some embodiments, the structural moiety

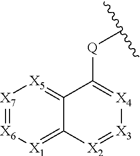

has the structure of

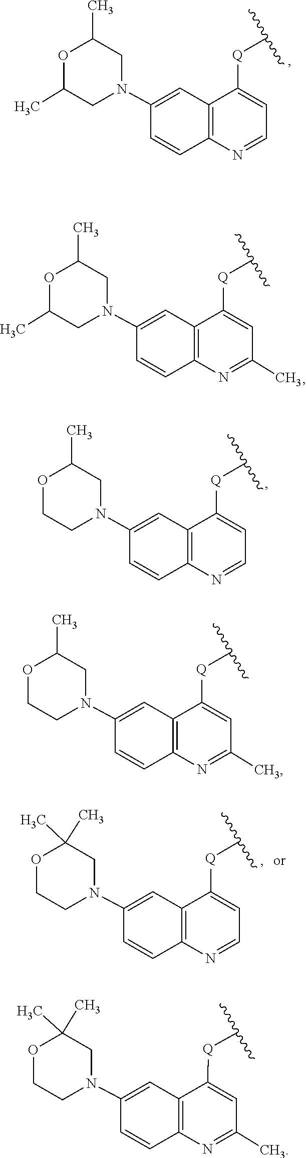

In some embodiments, the structural moiety

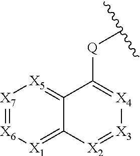

has the structure of

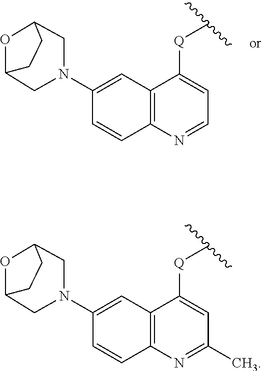

In some embodiments, the structural moiety

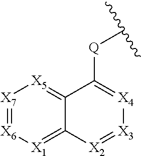

has the structure of

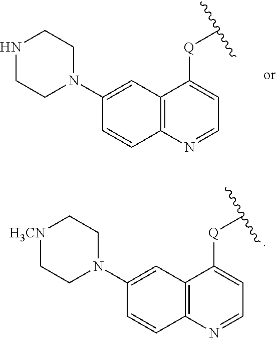

In some embodiments, the structural moiety

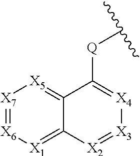

has the structure of

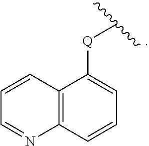

In some embodiments, the structural moiety

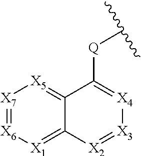

has the structure of

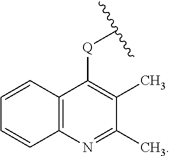

In some embodiments, the structural moiety

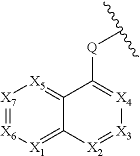

has the structure of

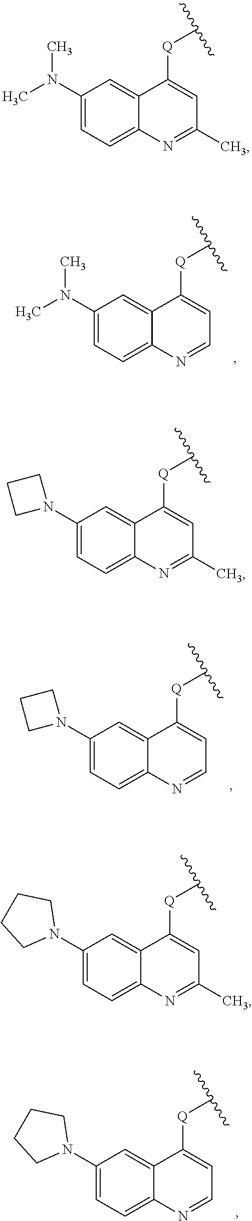

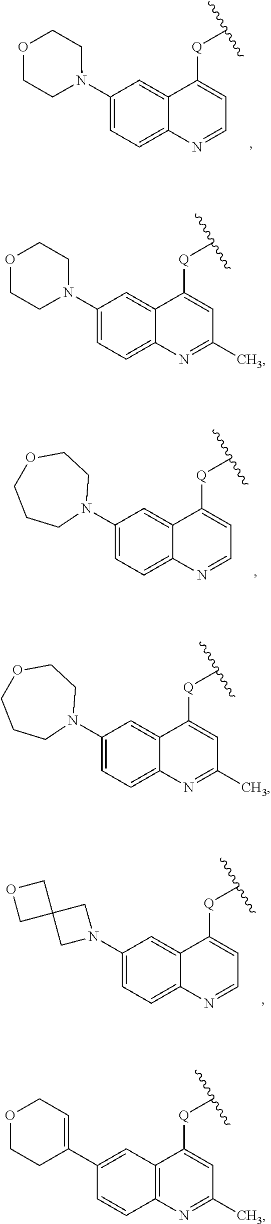

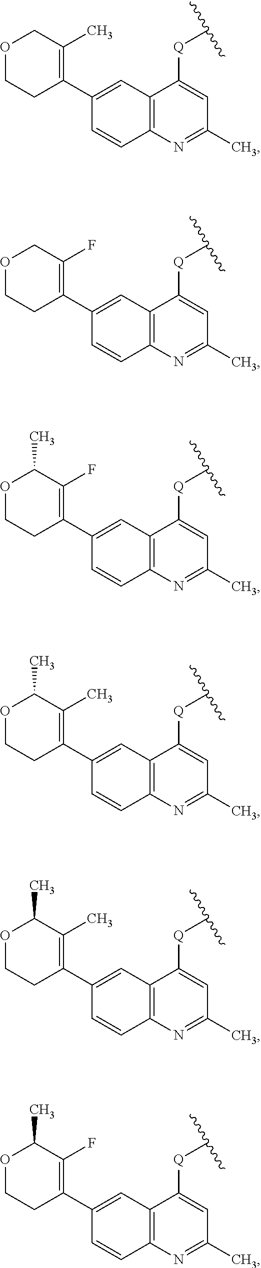

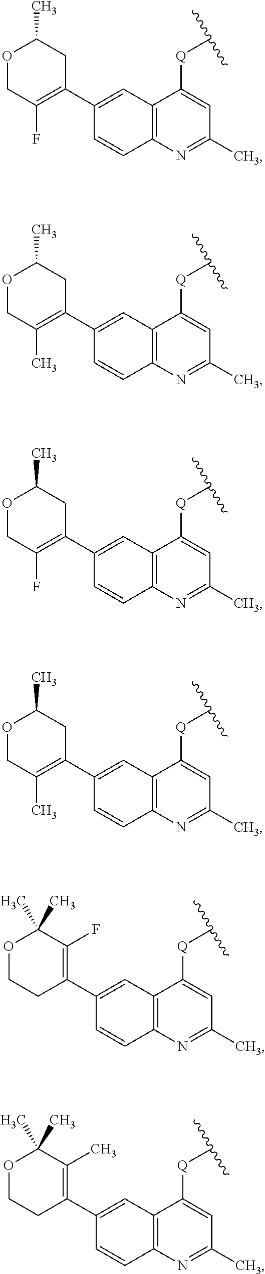

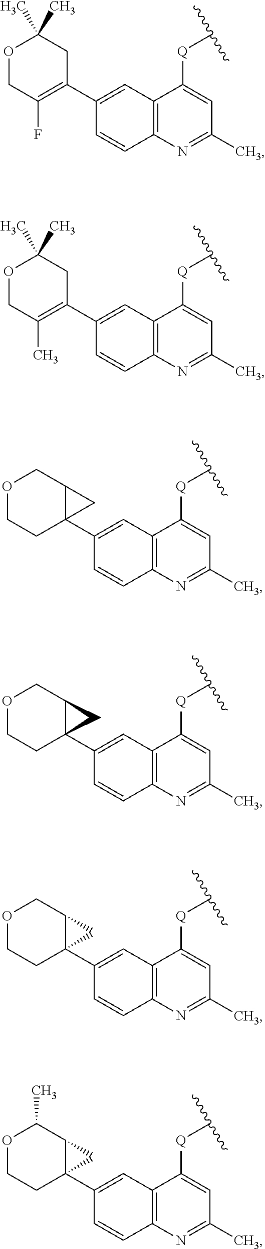

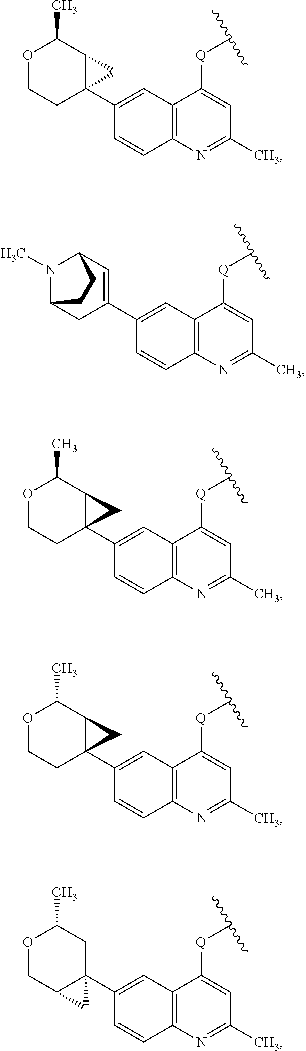

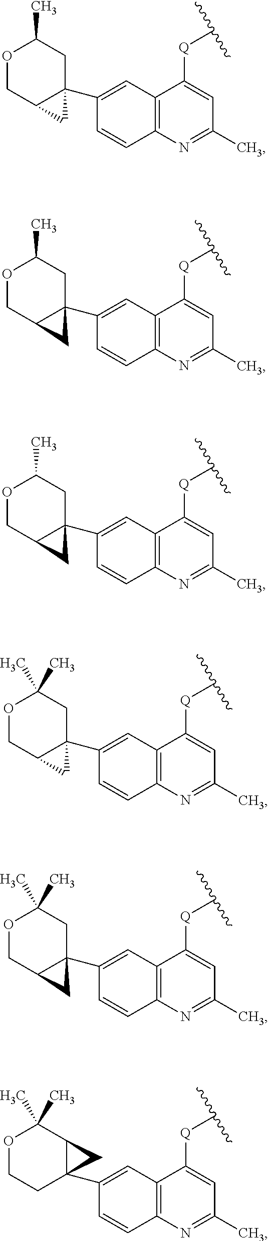

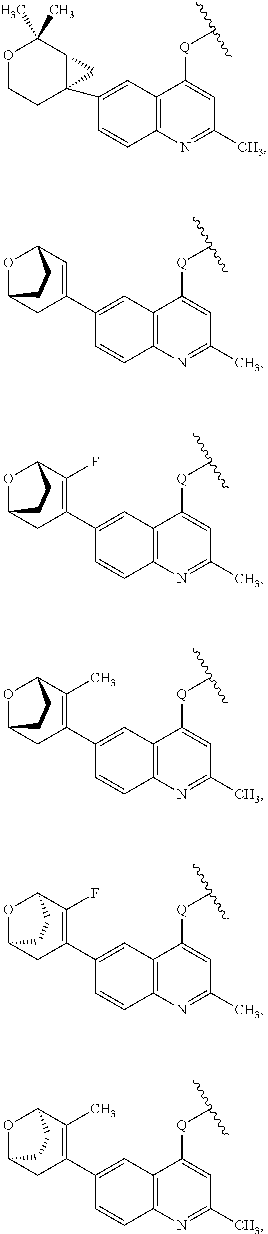

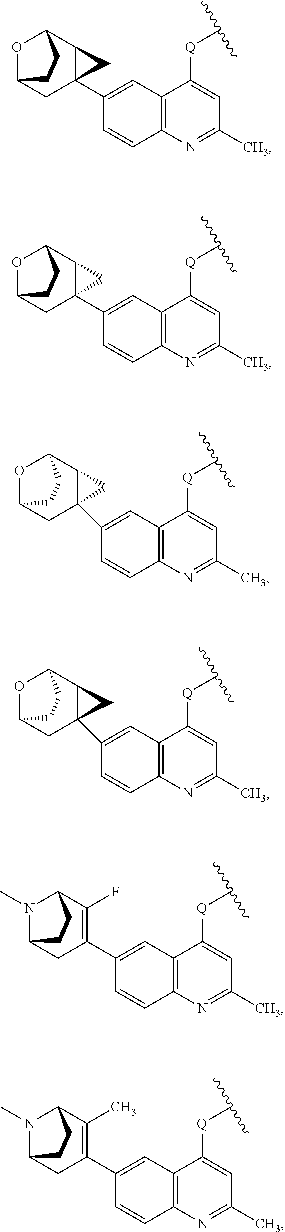

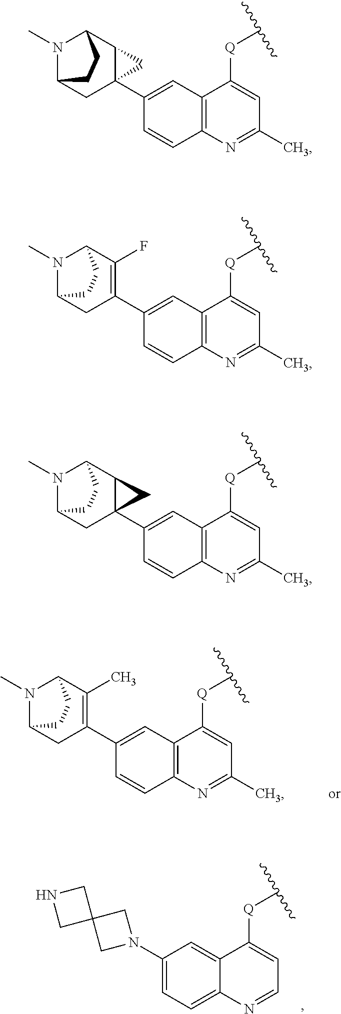

where Q is O or NH. In some embodiments, the structural moiety

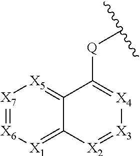

has the structure of

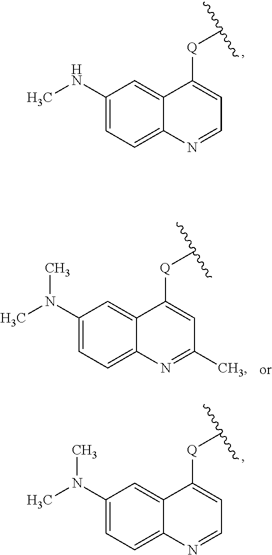

where Q is O or NH. In some embodiments, the structural moiety

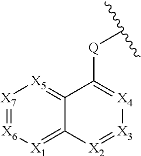

has the structure of

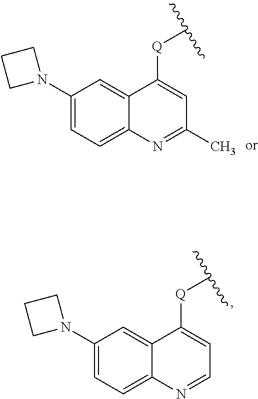

where Q is O or NH. In some embodiments, the structural moiety

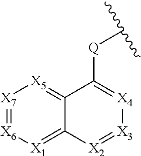

has the structure of

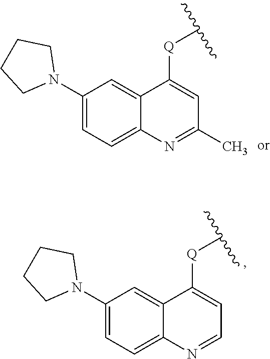

where Q is O or NH. In some embodiments, the structural moiety

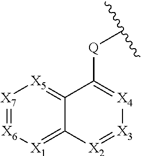

has the structure of

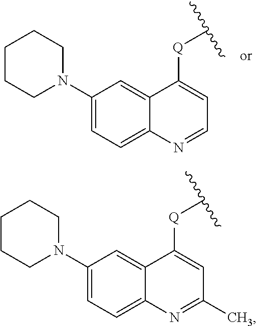

where Q is O or NH. In some embodiments, the structural moiety

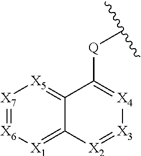

has the structure of

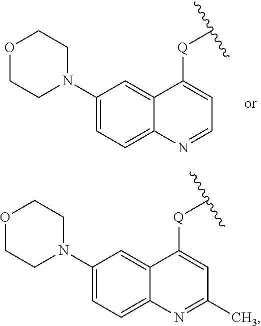

where Q is O or NH. In some embodiments, the structural moiety

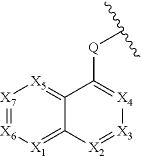

has the structure of

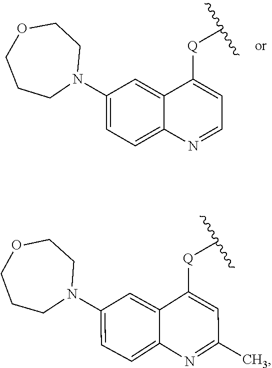

where Q is O or NH. In some embodiments, the structural moiety

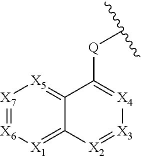

has the structure of where Q

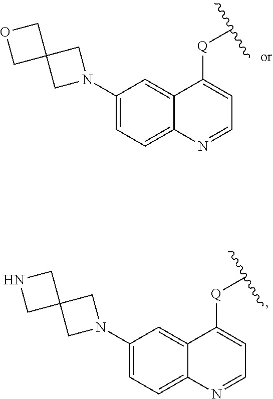

where Q is O or NH. In some embodiments, the structural moiety

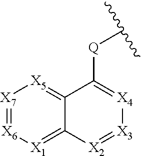

has the structure of

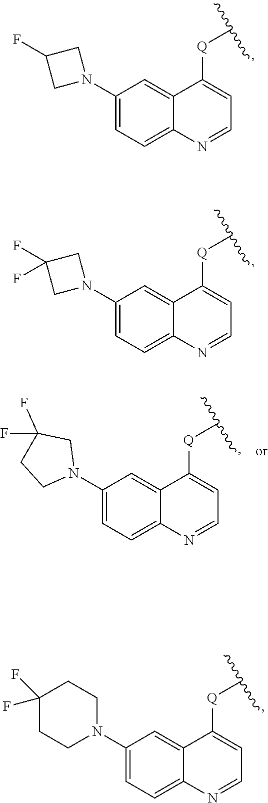

where Q is O or NH. In some embodiments, the structural moiety

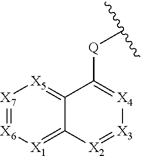

has the structure of

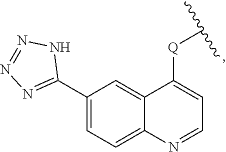

where Q is O or NH.

In some embodiments, the structural moiety

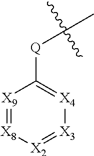

has the structure of

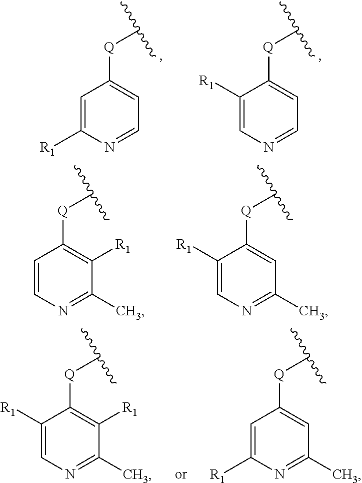

where Q is O or NH and R₁ is H, D, (C₁-C₆)alkyl, (C₃-C₇)heterocycloalkyl, halogenated (C₃-C₇)heterocycloalkyl, or halogen. In some embodiments, the structural moiety

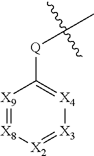

has the structure of

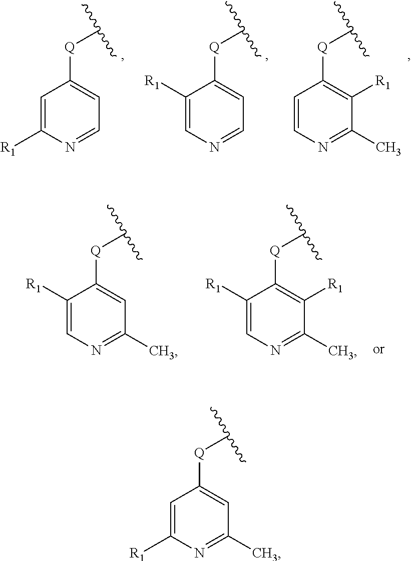

where Q is O or NH and R₁ is H, D, methyl, ethyl, propyl, isopropyl, butyl, isobutyl, sec-butyl, tert-butyl, F, Cl, or Br.

In some embodiments, the structural moiety

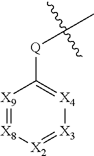

has the structure of

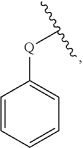

where Q is O or NH. In some embodiments, the structural moiety

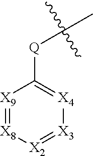

has the structure of

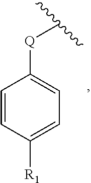

where Q is O or NH. In some embodiments, the structural moiety

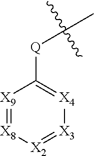

has the structure of

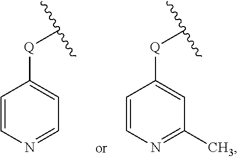

where Q is O or NH. In some embodiments, the
structural moiety

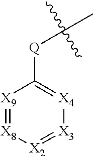

has the structure of

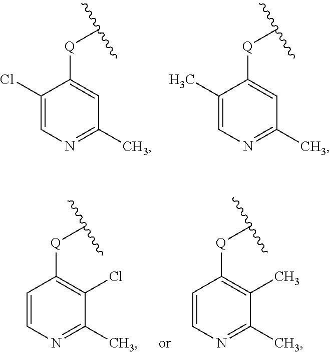

where Q is O or NH. In some embodiments, the structural moiety

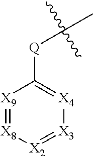

has the structure of

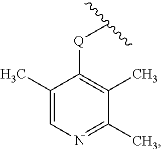

where Q is O or NH.

In some embodiments, the structural moiety

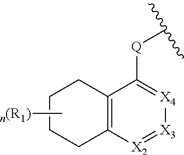

has the structure of

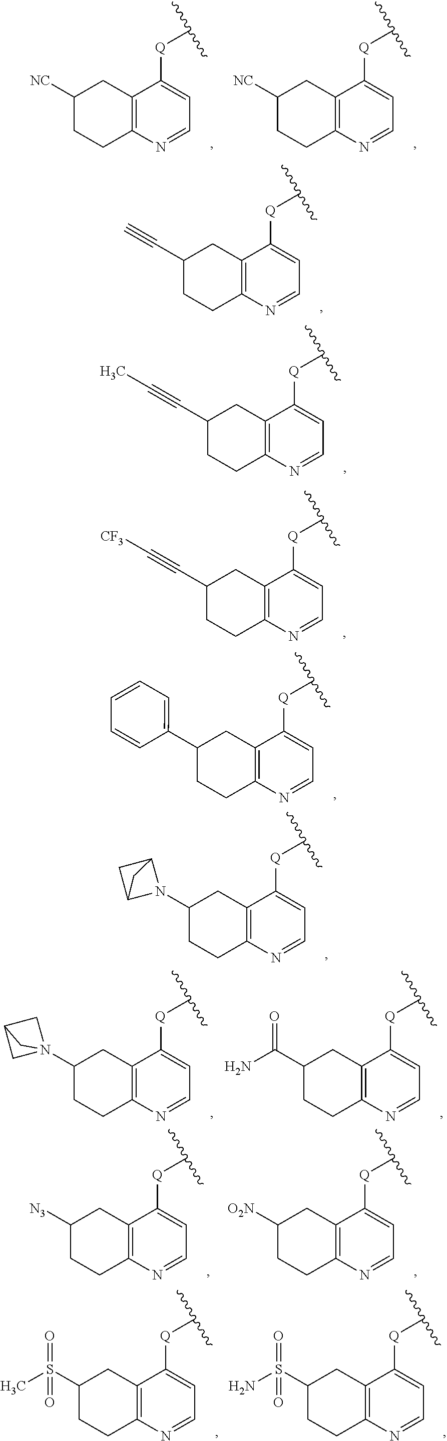

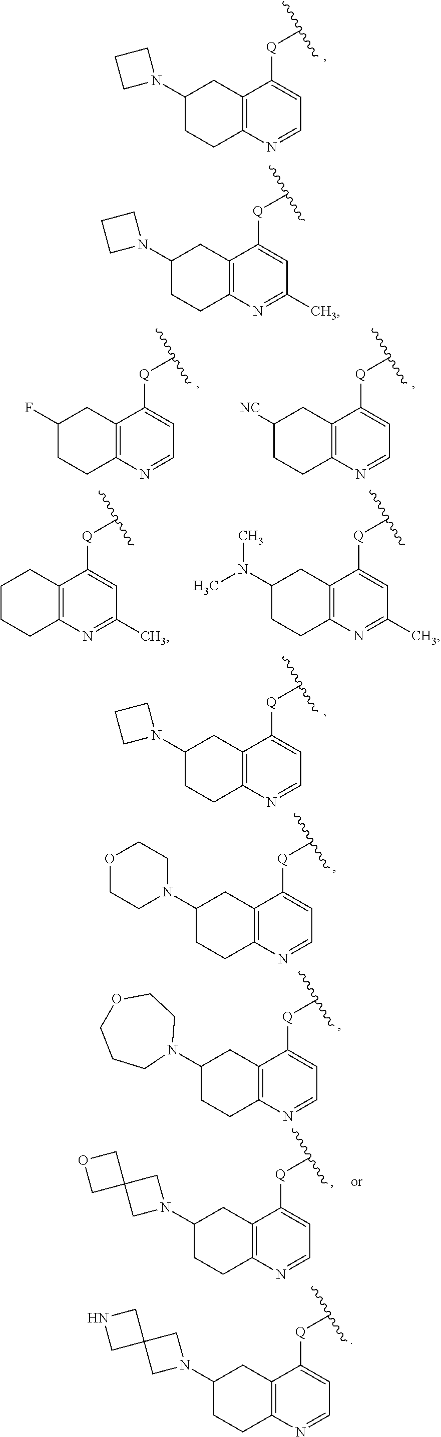

In some embodiments, the structural moiety

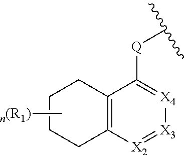

has the structure of

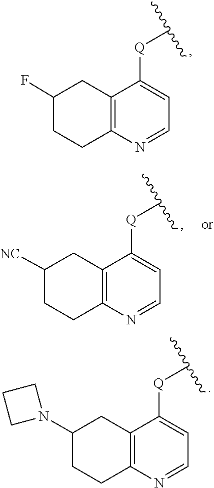

In some embodiments, the structural moiety

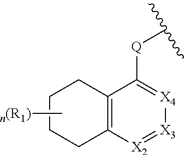

has the structure of

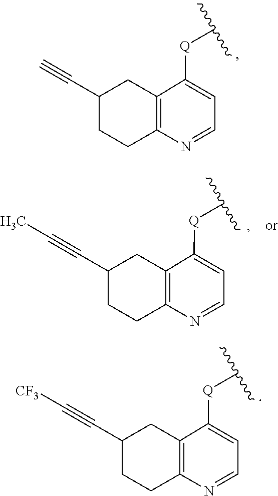

In some embodiments, the structural moiety

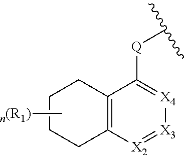

has the structure of

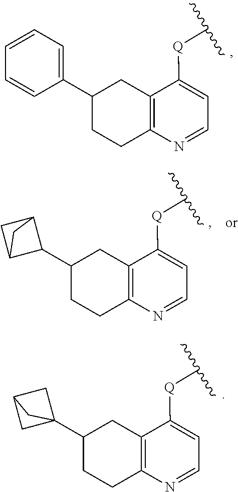

In some embodiments, the structural moiety

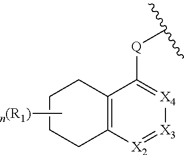

has the structure of

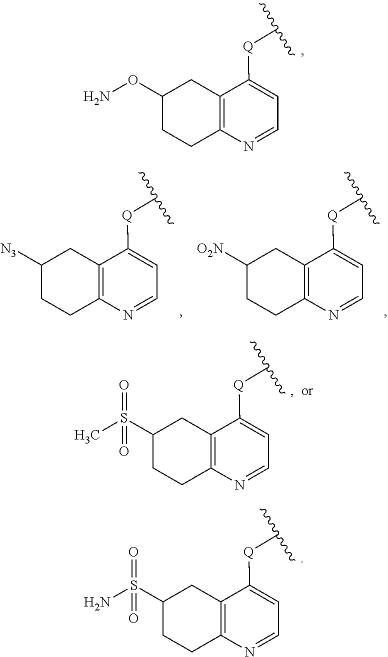

In some embodiments, the structural moiety

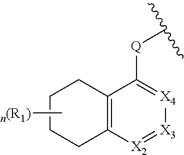

has the structure of

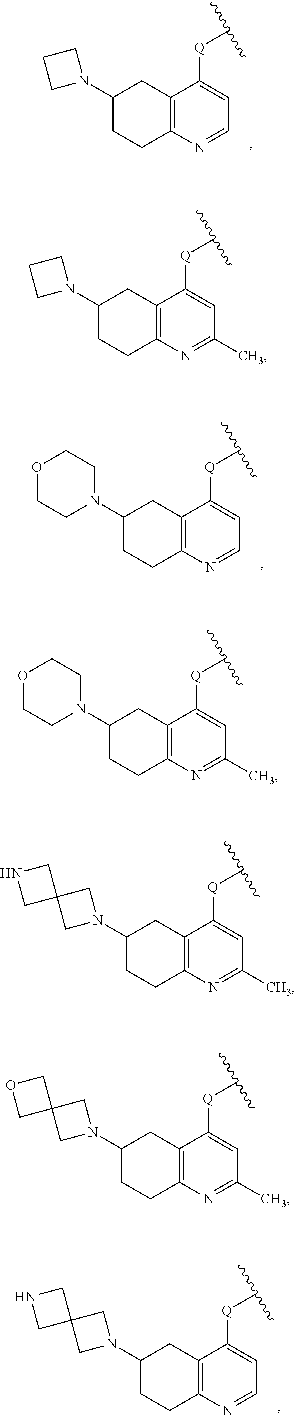

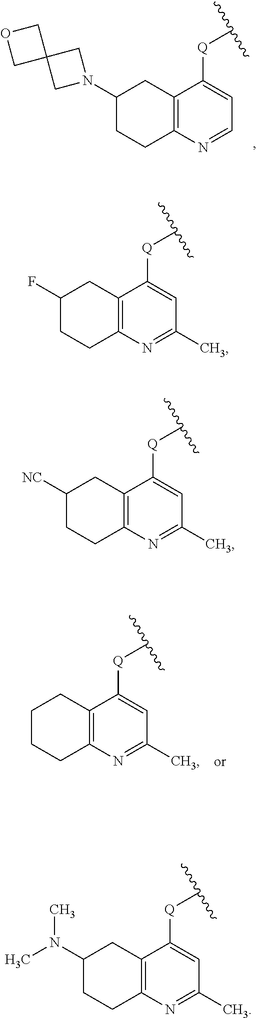

In some embodiments, Q is O or NH.

In some embodiments, the compound has the structure of Formula Ia.

In some embodiments, Y₁, Y₂, Y₃, Y₄, and Y₅ are each independently CR₂ or N. In some embodiments, Y₁, Y₂, Y₃, Y₄, and Y₅ are each CR₂. In some embodiments, Y₁, Y₂, Y₃, Y₄, and Y₅ are each CH. In some embodiments, Y₁, Y₂, Y₃, Y₄, and Y₅ are each N. In some embodiments, one of Y₁, Y₂, Y₃, Y₄, and Y₅ is CR₂ and the rest are N. In some embodiments, one of Y₁, Y₂, Y₃, Y₄, and Y₅ is CH and the rest are N. In some embodiments, two of Y₁, Y₂, Y₃, Y₄, and Y₅ are CR₂ and the rest are N. In some embodiments, two of Y₁, Y₂, Y₃, Y₄, and Y₅ are CH and the rest are N. In some embodiments, three of Y₁, Y₂, Y₃, Y₄, and Y₅ are CR₂ and two of Y₁, Y₂, Y₃, Y₄, and Y₅ are N. In some embodiments, three of Y₁, Y₂, Y₃, Y₄, and Y₅ are CH and two of Y₁, Y₂, Y₃, Y₄, and Y₅ are N.

In some embodiments, the structural moiety

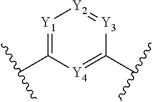

has the structure of

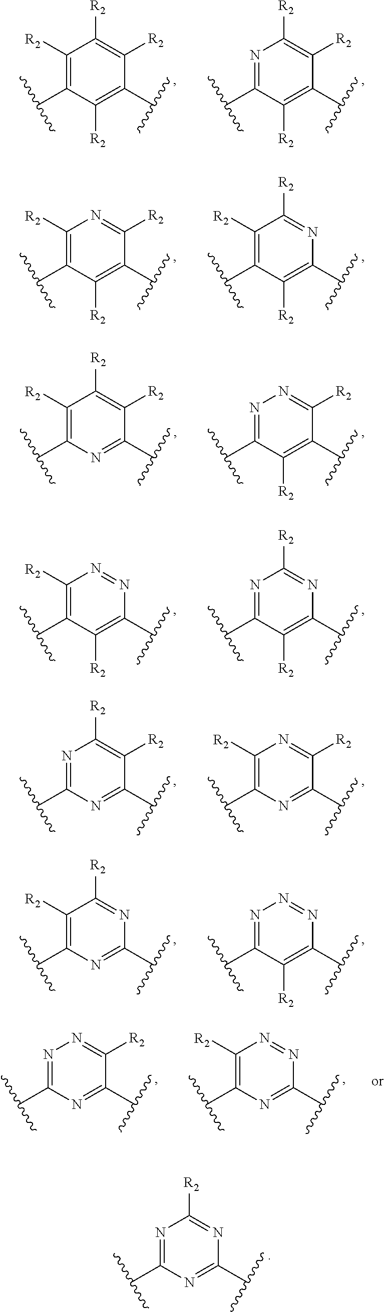

In some embodiments, the structural moiety

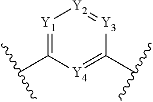

has the structure of

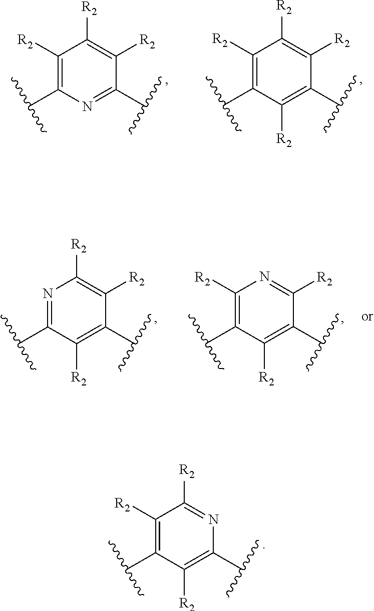

In some embodiments, each occurrence of R₂ is independently selected from the group consisting of H, D, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₇)cycloalkyl, (C₄-C₁₀)bicycloalkyl, (C₄-C₁₀)heterobicycloalkyl, (C₃-C₇)heterocycloalkyl, (C₄-C₁₀)heterobicycloalkyl, aryl, heteroaryl, —ORₐ, —N(Rₐ)₂, —CORₐ, —CO₂Rₐ, CON(Rₐ)₂, —CN, —NC, NO₂, N₃, —SO₂Rₐ, —SO₂N(Rₐ)₂, and —N(Rₐ)SO₂Rₐ. In some embodiments, each occurrence of R₂ is independently selected from the group consisting of (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₇)cycloalkyl, (C₄-C₁₀)bicycloalkyl, (C₃-C₇)heterocycloalkyl, and (C₄-C₁₀)heterobicycloalkyl. In some embodiments, each occurrence of R₂ is independently selected from the group consisting of aryl and heteroaryl. In some embodiments, each occurrence of R₂ is independently selected from the group consisting of —ORₐ, —SRₐ, —N(Rₐ)₂, —CORₐ, —CO₂Rₐ, CON(Rₐ)₂, —CN, —NC, NO₂, N₃, —SO₂Rₐ, —SO₂N(Rₐ)₂, and —N(Rₐ)SO₂Rₐ. In some embodiments, each occurrence of R₂ is independently selected from the group consisting of

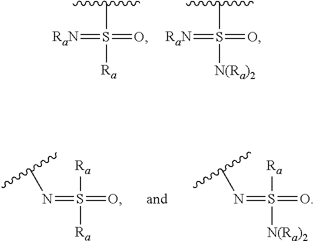

In some embodiments, each occurrence of R₂ is independently H, D, halogen, ORₐ, N(Rₐ)₂, (C₁-C₆)alkyl, (C₃-C₇)heterocycloalkyl, (C₁-C₆)alkynyl, aryl, (C₄-C₁₀)bicycloalkyl, —CN, —NC, N₃, NO₂, CORₐ, CO₂Rₐ, CON(Rₐ) 2, —SO₂Rₐ, or —SO₂N(Rₐ)₂. In some embodiments, each occurrence of R₂ is independently H, D, halogen, (C₁-C₆)alkyl, (C₃-C₇)heterocycloalkyl, N(Rₐ)₂, or —CN. In some embodiments, each occurrence of R₂ is independently (C₄-C₁₀)heterospiroalkyl, halogenated (C₃-C₇)heterocycloalkyl, aryl, or heteroaryl. In some embodiments, each occurrence of R₂ is independently H, (C₁-C₆)alkyl, (C₁-C₆)alkynyl, aryl, (C₄-C₁₀)bicycloalkyl, —SO₂Rₐ, or —SO₂N(Rₐ)₂. In some embodiments, each occurrence of R₂ is independently H, D, F, Cl, Br, CH₃, OCH₃, NH₂, N(CH₃)₂,

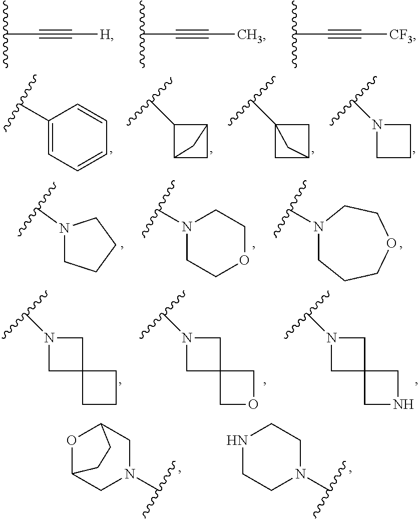

—CN, —NC, N₃, NO₂,

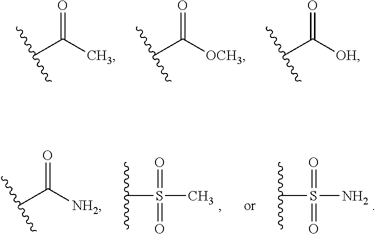

In some embodiments, each occurrence of R₂ is independently H, D, F, CH₃, N(CH₃)₂,

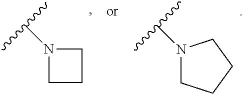 [SMILES: *N1CCC1 ; *N1CCCC1]

In some embodiments, at least one occurrence of R₂ is H, D, or halogen. In some embodiments, at least one occurrence of R₂ is H. In some embodiments, at least one occurrence of R₂ is D. In some embodiments, at least one occurrence of R₂ is F. In some embodiments, at least one occurrence of R₂ is CH₃. In some embodiments, at least one occurrence of R₂ is OCH₃. In some embodiments, at least one occurrence of R₂ is NH₂. In some embodiments, at least one occurrence of R₂ is N(CH₃)₂. In some embodiments, at least one occurrence of R₂ is

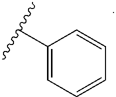 [SMILES: *c1ccccc1]

In some embodiments, at least one occurrence of R₂ is

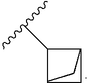 [SMILES: *C1CCC1]

In some embodiments, at least one occurrence of R₂ is

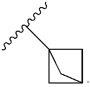 [SMILES: *C12CC(C1)C2]

In some embodiments, at least one occurrence of R₂ is

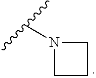 [SMILES: *N1CCC1]

In some embodiments, at least one occurrence of R₂ is

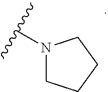 [SMILES: *N1CCCC1]

In some embodiments, at least one occurrence of R₂ is

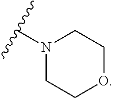 [SMILES: *N1CCOCC1]

In some embodiments, at least one occurrence of R₂ is

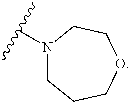 [SMILES: *N1CCCOCC1]

In some embodiments, at least one occurrence of R₂ is

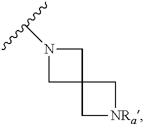

where Rₐ′ is H or (C₁-C₆)alkyl. In some embodiments, at least one occurrence of R₂ is

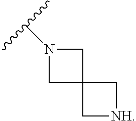 [SMILES: *N1CC2(CNC2)C1]

In some embodiments, at least one occurrence of R₂ is

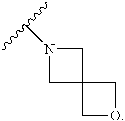 [SMILES: *N1CC2(COC2)C1]

In some embodiments, at least one occurrence of R₂ is

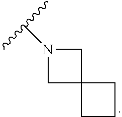 [SMILES: *N1CC2(CCC2)C1]

In some embodiments, at least one occurrence of R₂ is

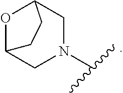 [SMILES: *N1CC2CCC(C1)O2]

In some embodiments, at least one occurrence of R₂ is

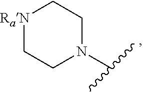

where Rₐ′ is H or (C₁-C₆)alkyl. In some embodiments, at least one occurrence of R₂ is

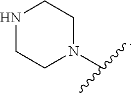 [SMILES: *N1CCNCC1]

In some embodiments, at least one occurrence of R₂ is

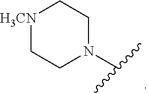

In some embodiments, at least one occurrence of R₂ is

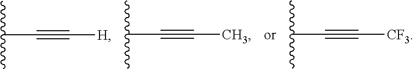

In some embodiments, at least one occurrence of R₂ is

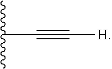

In some embodiments, at least one occurrence of R₂ is

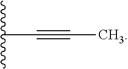 [SMILES: *C#CC]

In some embodiments, at least one occurrence of R₂ is

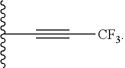 [SMILES: *C#CC(F)(F)F]

In some embodiments, at least one occurrence of R₂ is-CN. In some embodiments, at least one occurrence of R₂ is —NC. In some embodiments, at least one occurrence of R₂ is

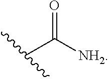 [SMILES: *C(N)=O]

In some embodiments, at least one occurrence of R₂ is

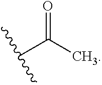 [SMILES: *C(C)=O]

In some embodiments, at least one occurrence of R₂ is

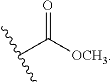 [SMILES: *C(=O)OC]

In some embodiments, at least one occurrence of R₂ is

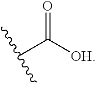 [SMILES: *C(=O)O]

In some embodiments, at least one occurrence of R₂ is NO₂. In some embodiments, at least one occurrence of R₂ is N₃. In some embodiments, at least one occurrence of R₂ is

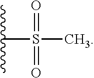 [SMILES: *S(C)(=O)=O]

In some embodiments, at least one occurrence of R₂ is

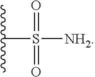 [SMILES: *S(N)(=O)=O]

In some embodiments, each occurrence of R₂ is independently selected from the group consisting of H, halogen, (C₁-C₆)alkyl, (C₁-C₆)haloalkyl, —N(Rₐ) 2, NO₂, and —ORₐ. In some embodiments, each occurrence of R₂ is independently H, halogen, CH₃, CF₃, OH, NH₂, —NHCH₃, or —N(CH₃)₂. In some embodiments, at least one occurrence of R₂ is H. In some embodiments, at least one occurrence of R₂ is (C₁-C₆)alkyl. In some embodiments, at least one occurrence of R₂ is —N(Rₐ)₂, NO₂, or —ORₐ. In some embodiments, at least one occurrence of R₂ is H, CH₃, OH, NH₂, or halogen. In some embodiments, at least one occurrence of R₂ is H. In some embodiments, at least one occurrence of R₂ is CF₃. In some embodiments, R₂ is H or CH₃.

In some embodiments, the structural moiety

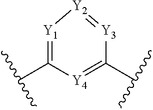

has the structure of

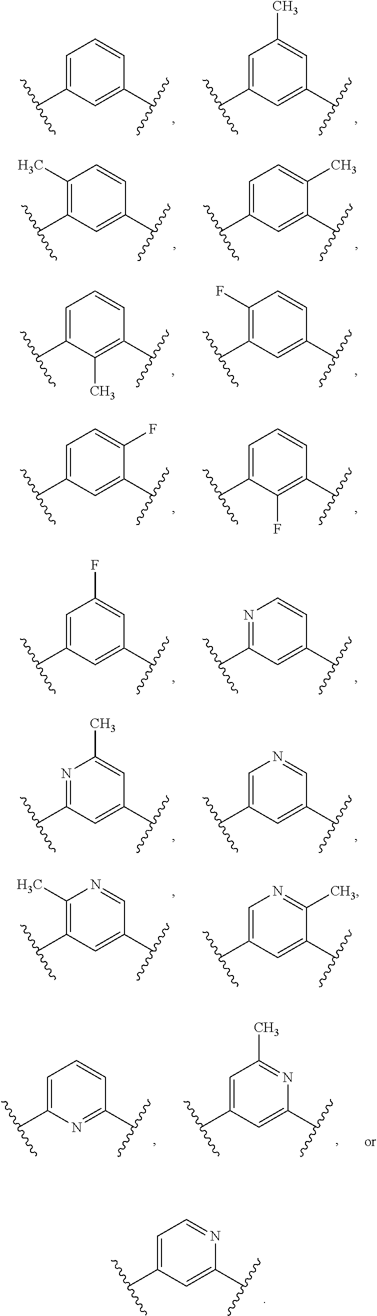

In some embodiments, Z₁, Z₂, Z₃, Z₄, and Z₅ are each independently CR₃ or N. In some embodiments, Z₁, Z₂, Z₃, Z₄, and Z₅ are each independently CR₃. In some embodiments, Z₁, Z₂, Z₃, Z₄, and Z₅ are each independently CH. In some embodiments, Z₁, Z₂, Z₃, Z₄, and Z₅ are each N. In some embodiments, one of Z₁, Z₂, Z₃, Z₄, and Z₅ is CR₃ and the rest are N. In some embodiments, one of Z₁, Z₂, Z₃, Z₄, and Z₅ is CH and the rest are N. In some embodiments, two of Z₁, Z₂, Z₃, Z₄, and Z₅ are CR₃ and the rest are N. In some embodiments, two of Z₁, Z₂, Z₃, Z₄, and Z₅ are CH and the rest are N. In some embodiments, three of Z₁, Z₂, Z₃, Z₄, and Z₅ are CR₃ and two are N. In some embodiments, three of Z₁, Z₂, Z₃, Z₄, and Z₅ are CH and two are N. In some embodiments, Z₄ is N and Z₁, Z₂, and Z₃, and Z₅ are CR₃.

In some embodiments, the structural moiety

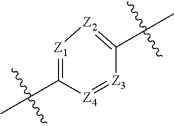

has the structure of

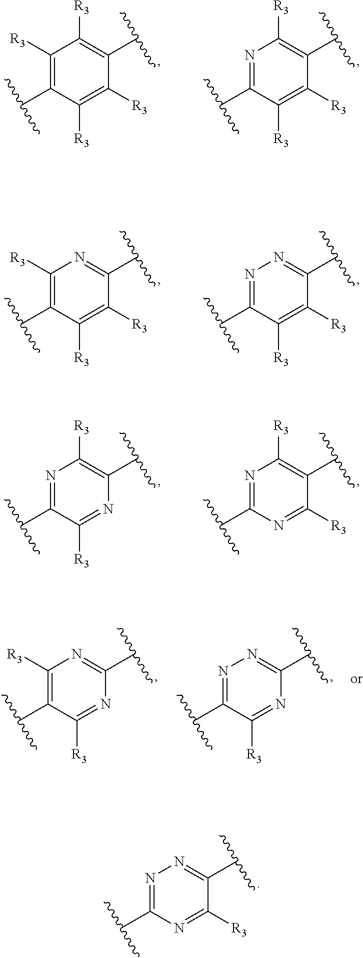

In some embodiments, the structural moiety

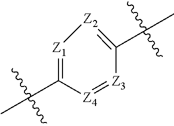

has the structure of

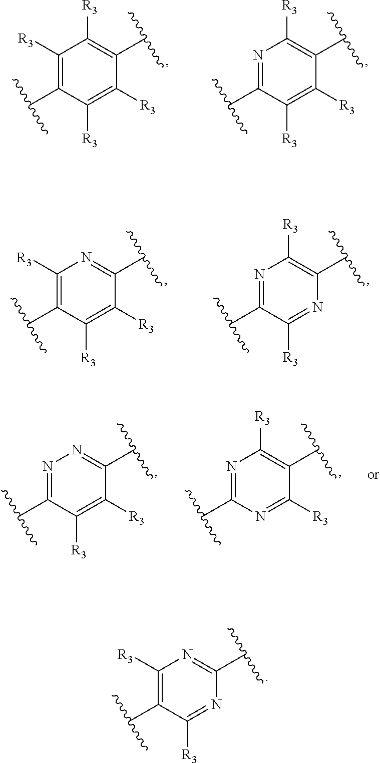

In some embodiments, the structural moiety

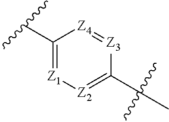

has the structure of

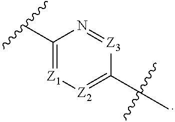

In some embodiments, the structural moiety

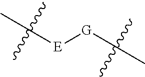

has the structure of

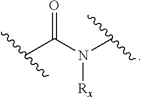

In some embodiments, the structural moiety

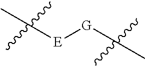

has the structure of

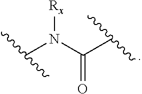

In some embodiments, the structural moiety

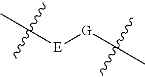

has the structure of

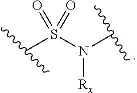

In some embodiments, the structural moiety

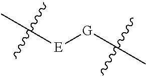

has the structure of

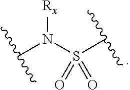

In some embodiments, the structural moiety

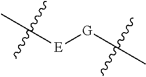

has the structure of

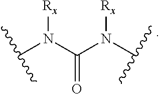

In some embodiments, the structural moiety

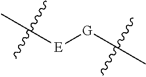

has the structure of

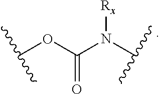

In some embodiments, the structural moiety

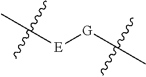

has the structure of

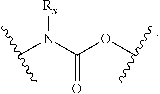

In these embodiments, the structural moiety

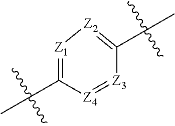

has the structure of

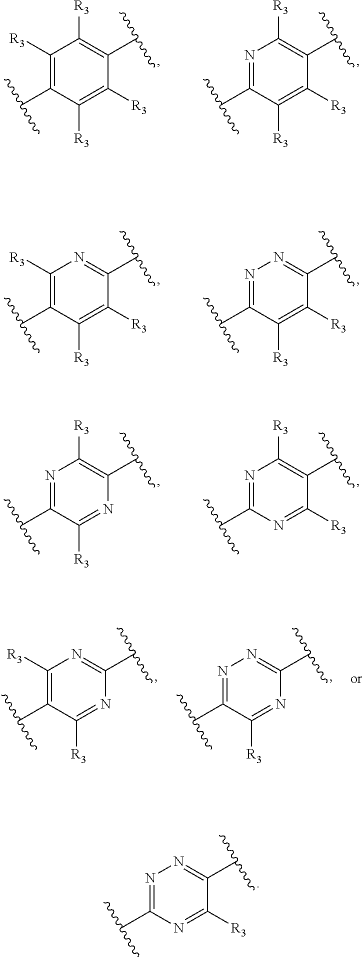

In some embodiments, each occurrence of Rₓ is independently H, (C₁-C₆)alkyl, (C₃-C₇)cycloalkyl, aryl, or heteroaryl; or where Rₓ and Y₃, Rₓ and Y₄, Rₓ and Z₁, or Rₓ and Z₄ taken together form an optionally substituted 5-6-membered heterocycle. In some embodiments, each occurrence of Rₓ is independently H, (C₁-C₆)alkyl, (C₃-C₇)cycloalkyl, aryl, or heteroaryl. In some embodiments, each occurrence of Rₓ is independently H, CH₃, or CH₂CH₃. In some embodiments, Rₓ and Y₄ taken together form an optionally substituted 5-6-membered heterocycle. In some embodiments, Rₓ and Y₃ taken together form an optionally substituted 5-6-membered heterocycle. In some embodiments, Rₓ and Z₁ taken together form an optionally substituted 5-6-membered heterocycle. In some embodiments, Rₓ and Z₄ taken together form an optionally substituted 5-6-membered heterocycle.

In some embodiments, the structural moiety

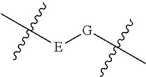

has the structure of

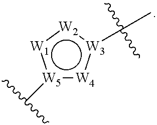

In some embodiments, W₁, W₂, W₃, W₄, and W₅ are each independently CR₆, N, or NR₆ where valence permits. In some embodiments, one of W₁, W₂, W₃, W₄, and W₅ are N or NR₆ and the rest are C or CR₆ where valence permits. In some embodiments, two of W₁, W₂, W₃, W₄, and W₅ are N or NR₆ and the rest are C or CR₆ where valence permits. In some embodiments, three of W₁, W₂, W₃, W₄, and W₅ are N or NR₆ and two are C or CR₆ where valence permits. In some embodiments, one of W₁, W₂, W₃, W₄, and W₅ are N and the rest are C or CR₆ where valence permits. In some embodiments, two of W₁, W₂, W₃, W₄, and W₅ are N and the rest are C or CR₆ where valence permits. In some embodiments, three of W₁, W₂, W₃, W₄, and W₅ are N and two are C or CR₆ where valence permits.

In some embodiments, each occurrence of R₆ is independently selected from the group consisting of H, halogen, (C₁-C₆)alkyl, and (C₁-C₆)haloalkyl. In some embodiments, each occurrence of R₆ is independently selected from the group consisting of H, F, CH₃, and CH₂CH₃.

In some embodiments, the structural moiety

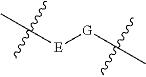

has the structure of

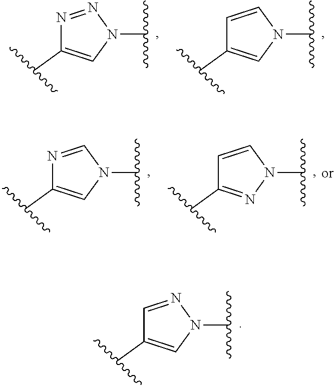 [SMILES: *c1cn(*)nn1 ; *c1ccn(*)c1 ; *c1cn(*)cn1 ; *c1ccn(*)n1 ; *c1cnn(*)c1]

In some embodiments, the structural moiety

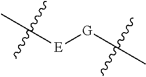

has the structure of

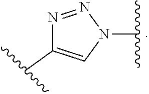 [SMILES: *c1cn(*)nn1]

In some embodiments, the structural moiety

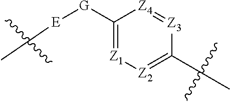

has the structure of

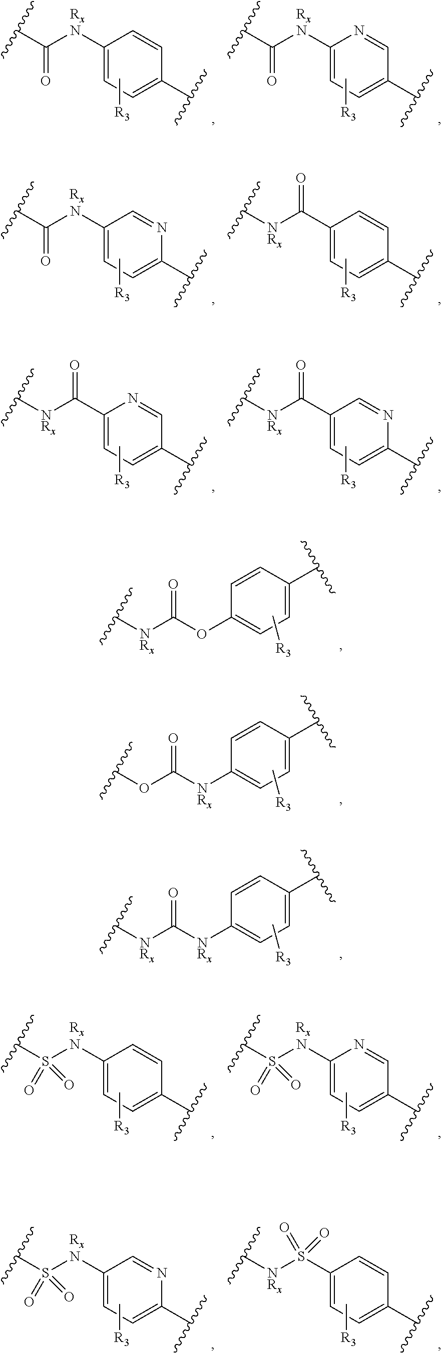

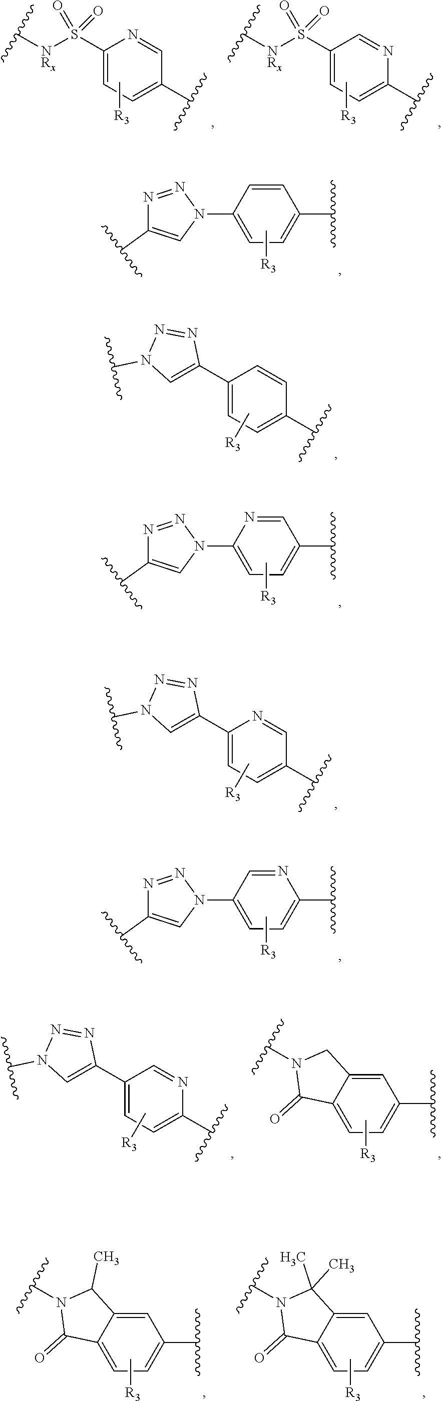

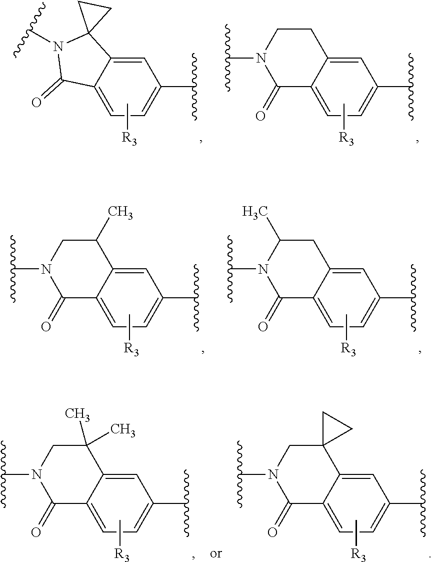

In some embodiments, R₃ is H, CH₃, OH, halogen, or NH₂. In some embodiments, Rₓ is H, CH₃, or CH₂CH₃.

In some embodiments, the structural moiety

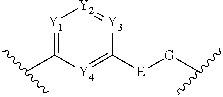

has the structure of

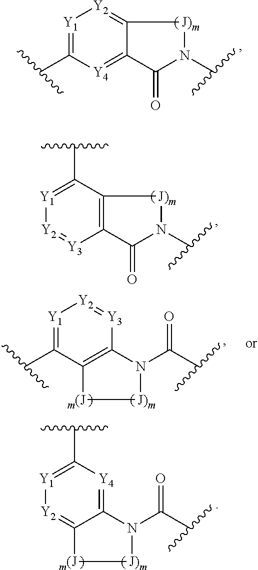

In some embodiments, the structural moiety

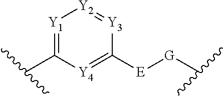

has the structure of

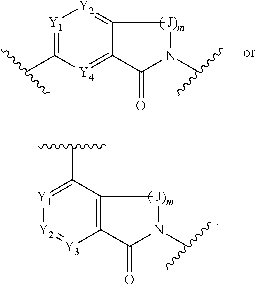

In some embodiments, the structural moiety

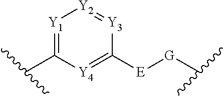

has the structure of

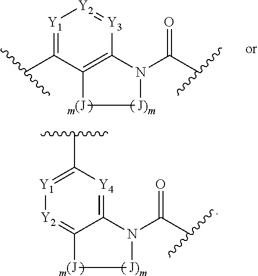

In these embodiments, each occurrence of m is independently 1 or 2, J is C(R_{y})₂, and each occurrence of R_{y} is independently H, (C₁-C₆)alkyl, OH, O(C₁-C₆)alkyl, or halogen; or any two R_{y} groups taken together with the carbon atom(s) they are connected to form a (C₃-C₇)cycloalkyl or (C₃-C₇)heterocycloalkyl. In some embodiments, m is 1. In some embodiments, m is 2. In some embodiments, each occurrence of R_{y} is independently H or (C₁-C₆)alkyl. In some embodiments each occurrence of R_{y} is independently OH, O(C₁-C₆)alkyl, or halogen. In some embodiments, each occurrence of R_{y} is H. In some embodiments, any two R_{y} groups taken together with the carbon atom(s) they are connected to form a (C₃-C₇)cycloalkyl or (C₃-C₇)heterocycloalkyl.

In some embodiments, the structural moiety

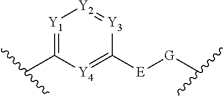

has the structure of

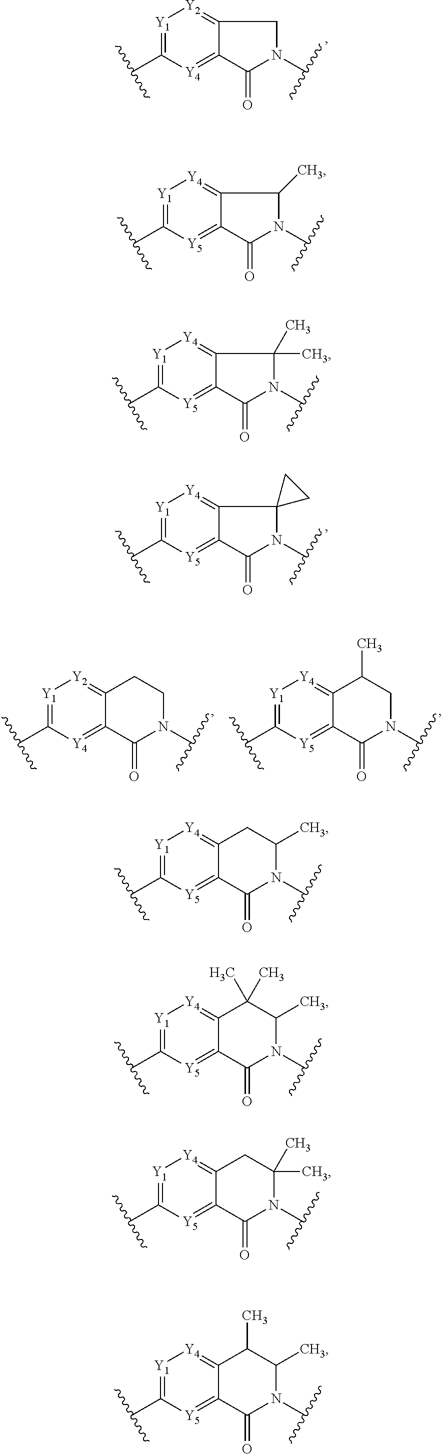

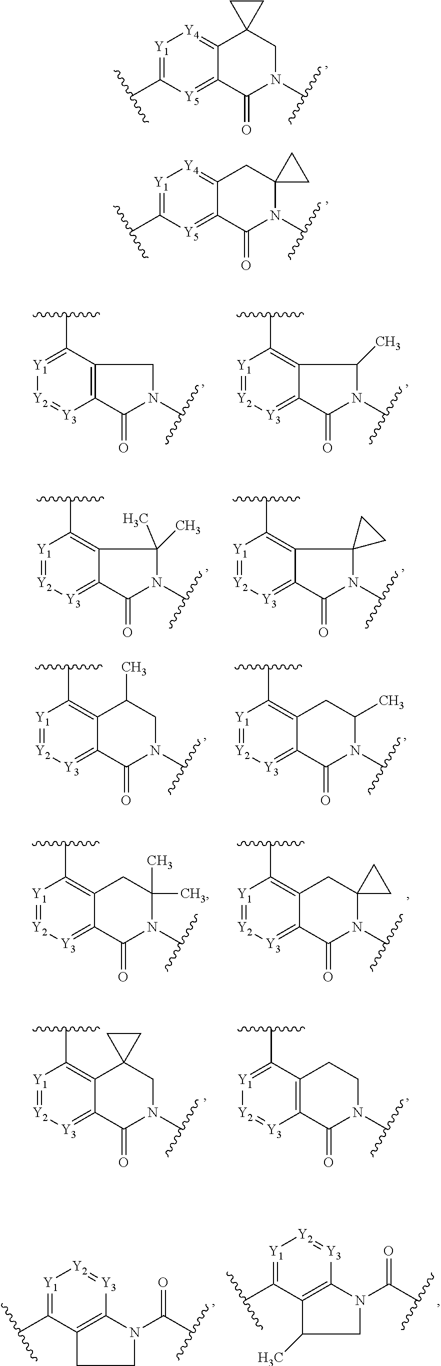

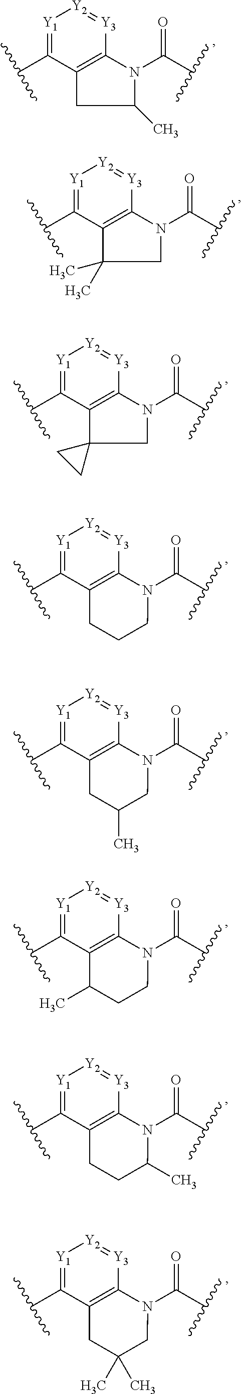

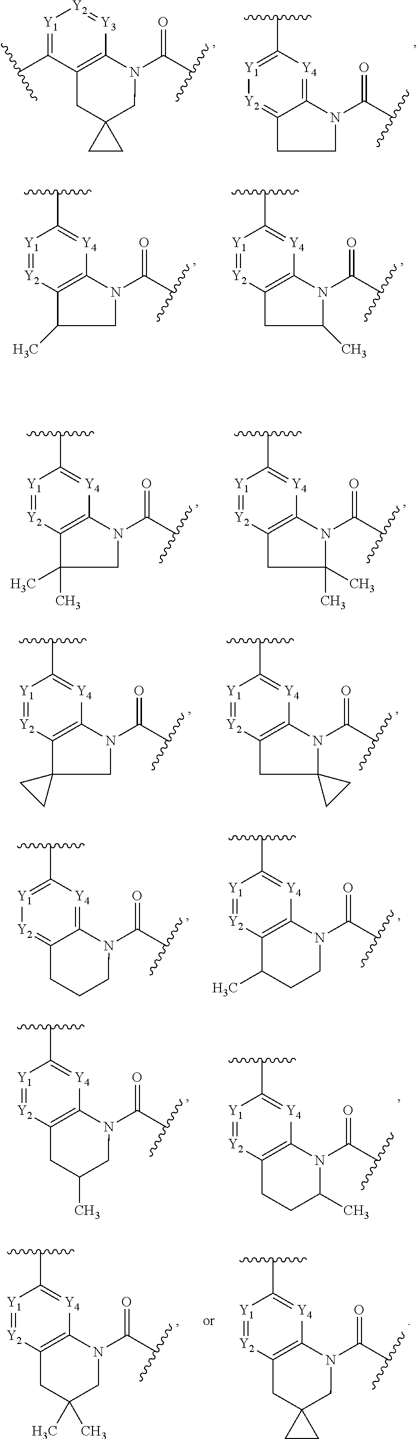

In some embodiments, the structural moiety

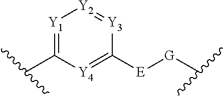

has the structure of

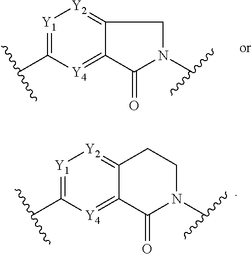

In some embodiments, the structural moiety

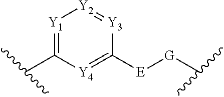

has the structure of

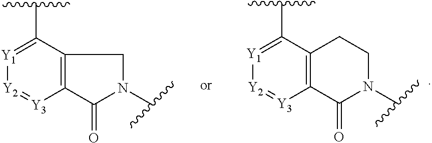

In some embodiments, the structural moiety

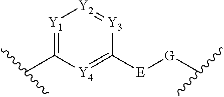

has the structure of

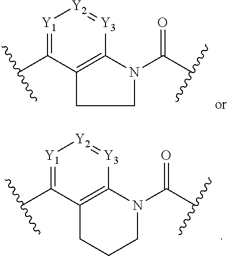

In some embodiments, the structural moiety

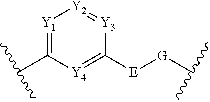

has the structure of

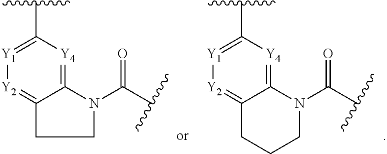

In some embodiments, Y₁, Y₂, Y₃, and Y₄ are each independently N, CH, CCH₃, or CF. In some embodiments, Y₁, Y₂, Y₃, and Y₄ are each independently N or CH.

In some embodiments, the structural moiety

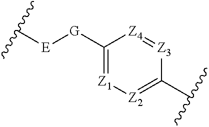

has the structure of

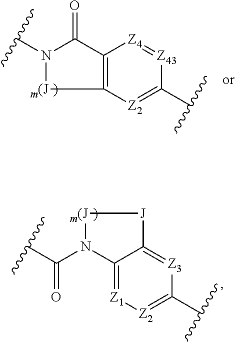

wherein each occurrence of m is independently 1 or 2, J is C(R_{z})₂, and each occurrence of R_{z} is independently H, (C₁-C₆)alkyl, OH, O(C₁-C₆)alkyl, or halogen; or any two R_{z} groups taken together with the carbon atom(s) they are connected to form a (C₃-C₇)cycloalkyl or (C₃-C₇)heterocycloalkyl. In some embodiments, m is 1. In some embodiments, m is 2. In some embodiments, each occurrence of R_{z} is independently H or (C₁-C₆)alkyl. In some embodiments, each occurrence of R_{z} is independently OH, O(C₁-C₆)alkyl, or halogen. In some embodiments, each occurrence of R_{z} is H. In some embodiments, each occurrence of R_{z} is CH₃. In some embodiments, two R_{z} groups taken together with the carbon atom(s) they are connected to form a (C₃-C₇)cycloalkyl. In some embodiments, two R_{z} groups taken together with the carbon atom(s) they are connected to form a (C₃-C₇)heterocycloalkyl. In some embodiments, two R_{z} groups taken together with the carbon atom they are connected to form a cyclopropyl.

In some embodiments, the structural moiety

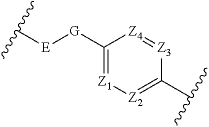

has the structure of

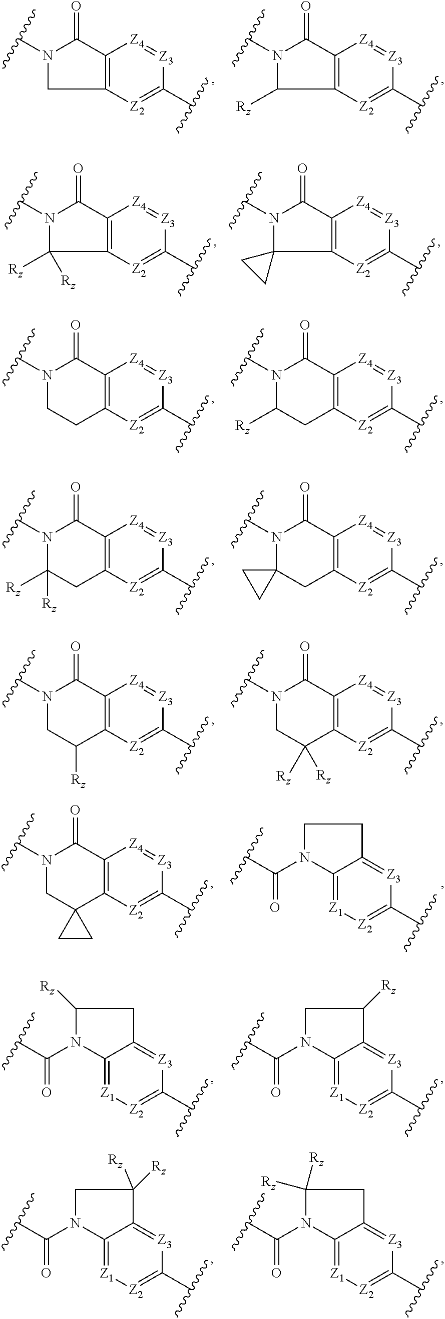

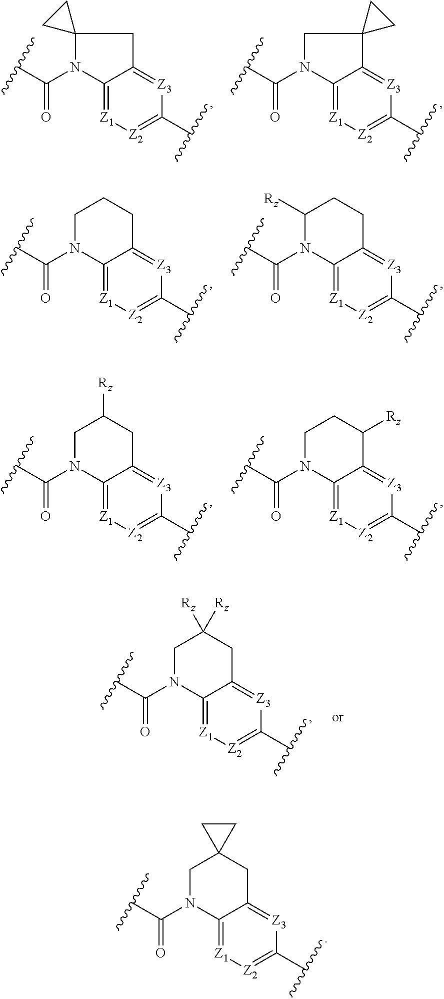

In some embodiments, the structural moiety

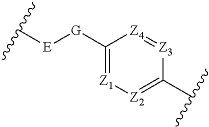

has the structure of

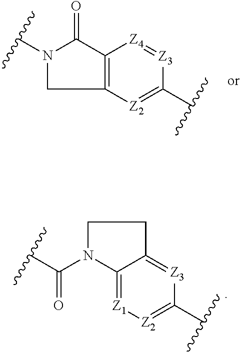

In some embodiments, the structural moiety

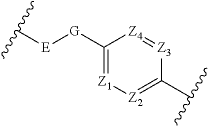

has the structure of

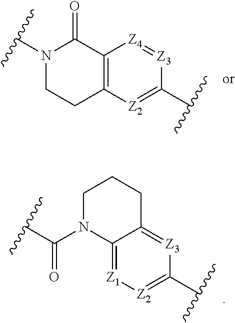

In some embodiments, the structural moiety

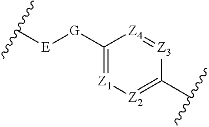

has the structure of

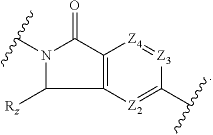

In some embodiments, the structural moiety

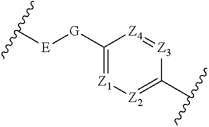

has the structure of

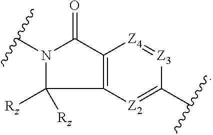

In some embodiments, the structural moiety

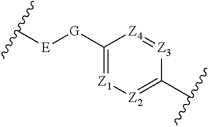

has the structure of

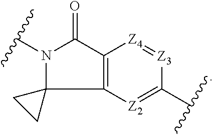

In some embodiments, Z₁, Z₂, Z₃, and Z₄ are each independently N, CH, CCH₃, or CF. In some embodiments, Z₁, Z₂, Z₃, and Z₄ are each independently N or CH. In some embodiments, each occurrence of R_{z} is independently H, or CH₃. In some embodiments, two R_{z} groups taken together with the carbon atom(s) they are connected to form a (C₃-C₇)cycloalkyl. In some embodiments, two R_{z} groups taken together with the carbon atom(s) they are connected to form a (C₃-C₇)heterocycloalkyl. In some embodiments, two R_{z} groups taken together with the carbon atom they are connected to form a cyclopropyl. In some embodiments, the structural moiety

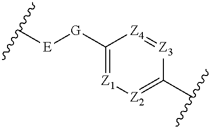

has the structure of

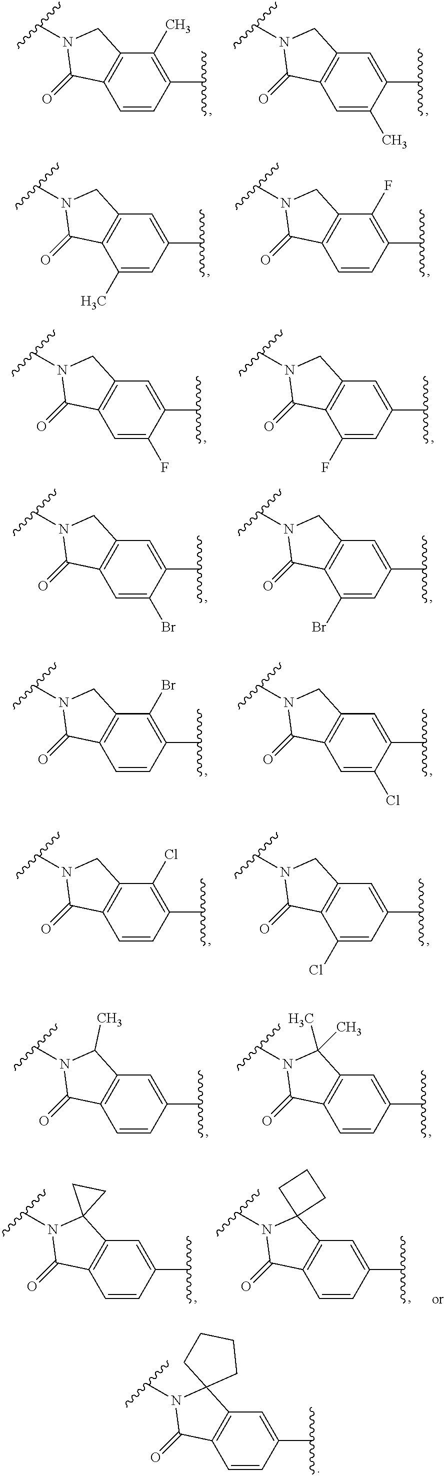

In some embodiments, the compound has the structure of Formula Ib.

In some embodiments, T is N(C═O)Rₐ or NSO₂Rₐ. In some embodiments, T is N(C═O)Me or N(C═O)Et. In some embodiments, T is NSO₂Me or NSO₂Et. In some embodiments, T is O or NRₐ. In some embodiments, T is O. In some embodiments, T is NRₐ. In some embodiments, T is NH. In some embodiments, T is NCH₃ or NCH₂CH₃. In some embodiments, T is NC(R_{b})₂OP(═O)(OR_{b})₂. In some embodiments, T is N or O, where valence permits.

In some embodiments, U is N(C═O)Rₐ or NSO₂Rₐ. In some embodiments, U is N(C═O) Me or N(C═O) Et. In some embodiments, U is NSO₂Me or NSO₂Et. In some embodiments, U is O or NRₐ. In some embodiments, U is O. In some embodiments, U is NRₐ. In some embodiments, U is NH. In some embodiments, U is NCH₃ or NCH₂CH₃. In some embodiments, U is NC(R_{b})₂OP(═O)(OR_{b})₂. In some embodiments, U is N or O, where valence permits.

In some embodiments, each occurrence of R_{b} is independently H or (C₁-C₆)alkyl. In some embodiments, each occurrence of R_{b} is independently H, methyl, ethyl, propyl, isopropyl, butyl, sec-butyl, isobutyl, or tert-butyl. In some embodiments, each occurrence of R_{b} is independently H or CH₃. In some embodiments, each occurrence of R_{b} is H.

In some embodiments, the structural moiety

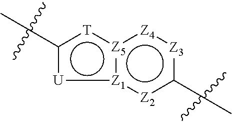

has the structure of

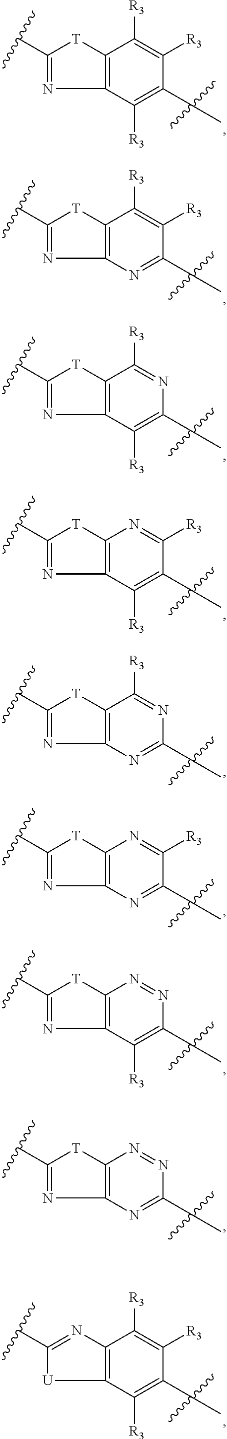

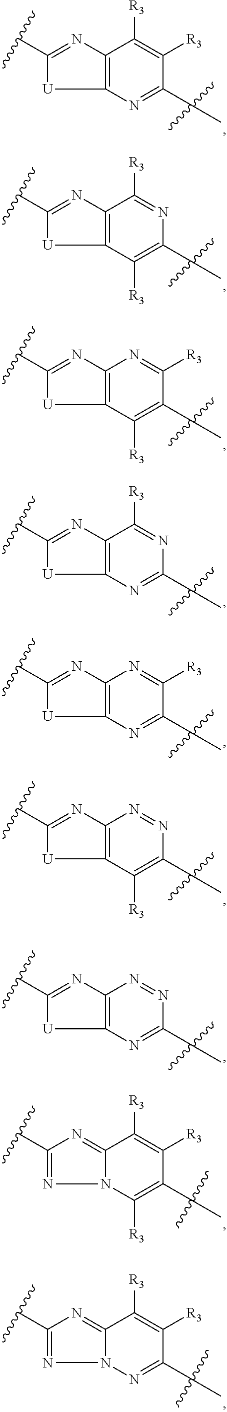

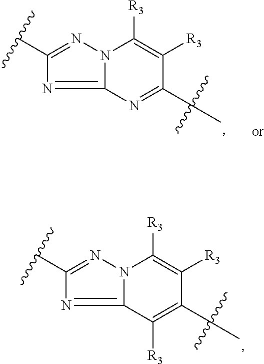

where each occurrence of T and U is independently O, N, NRₐ, N(CO)Rₐ, NC(R_{b})₂OP(═O)(OR_{b})₂, or NSO₂Rₐ where valance permits.

In some embodiments, the structural moiety

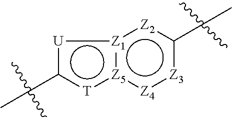

has the structure of

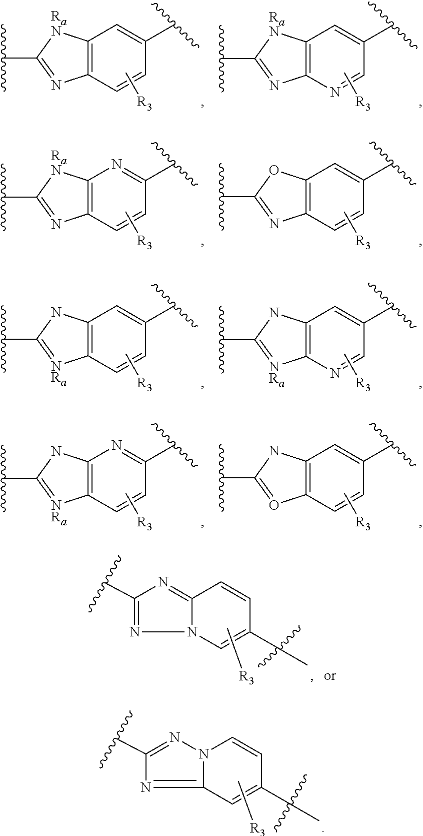

In some embodiments, the structural moiety

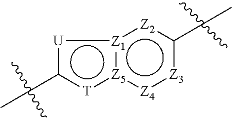

has the structure of

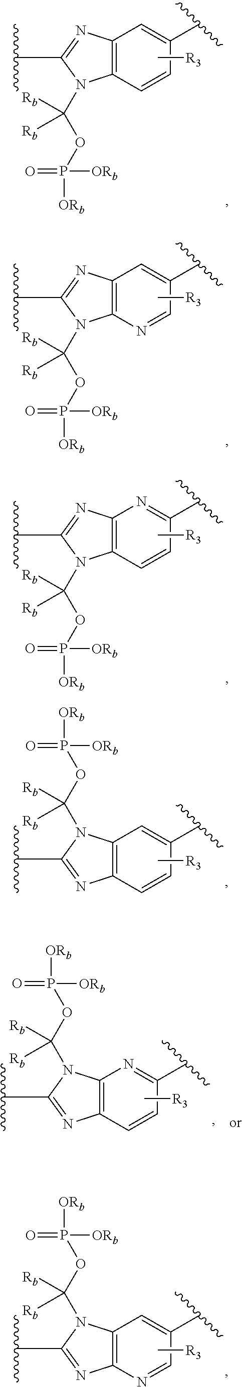

where each occurrence of R_{b} is independently H or CH₃. In some embodiments, the structural moiety

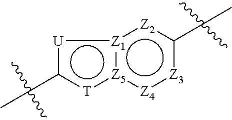

has the structure of

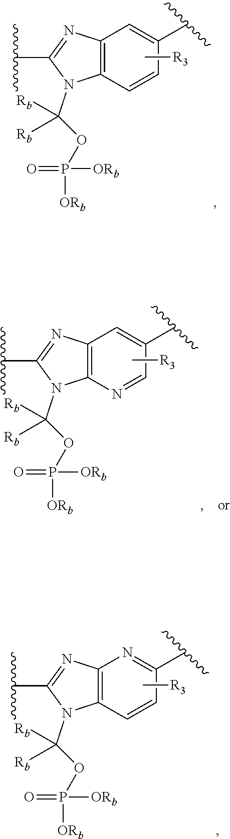

where each occurrence of R_{b} is independently H or CH₃. In some embodiments, the structural moiety

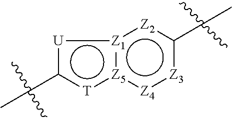

has the structure of

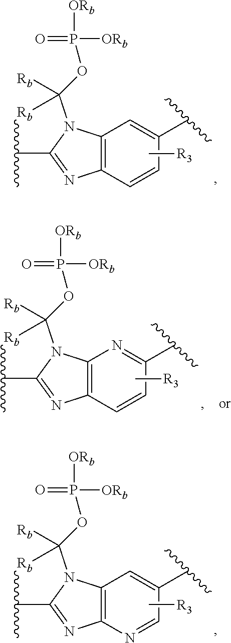

where each occurrence of R_{b} is independently H or CH₃.

In some embodiments, the compound has the structure of Formula Ic.

In some embodiments, the structural moiety

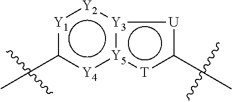

has the structure of

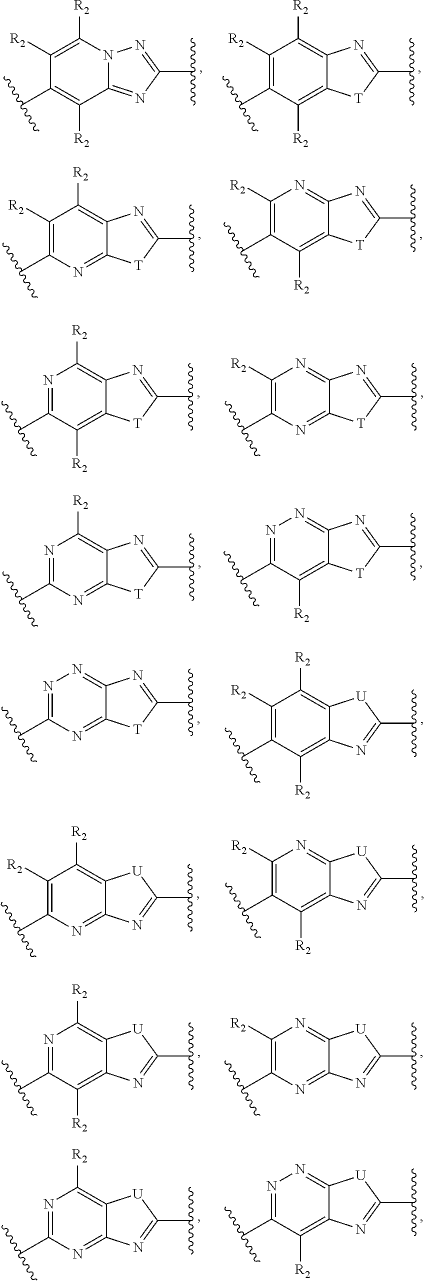

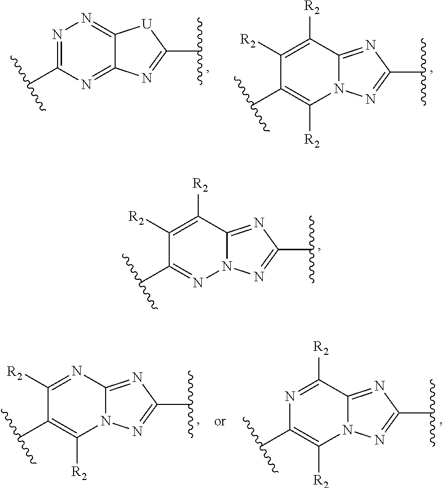

where each occurrence of T and U is independently O, N, NRₐ, N(C═O)Rₐ, NC(R_{b})₂OP(═O)(OR_{b})₂, or NSO₂Rₐ where valance permits.

In some embodiments, the structural moiety

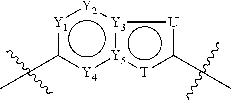

has the structure of

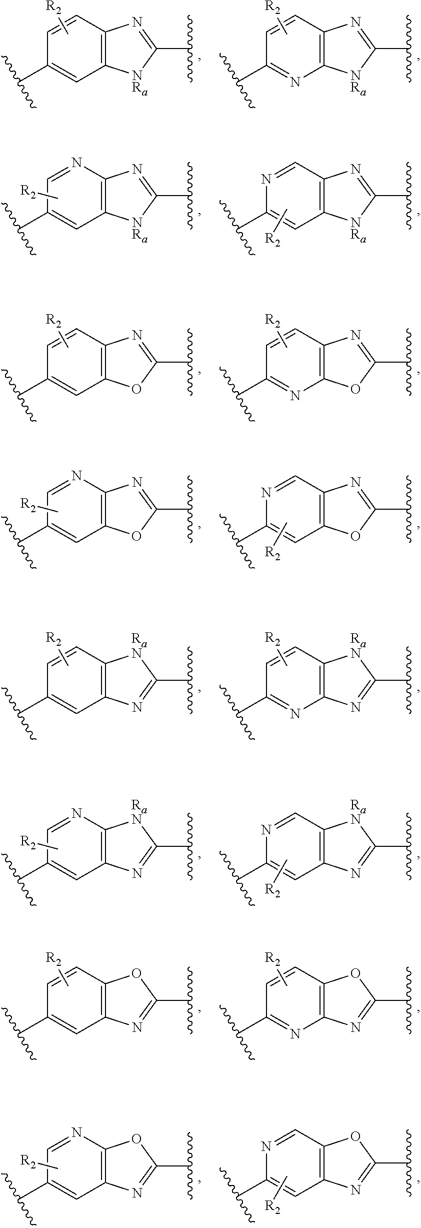

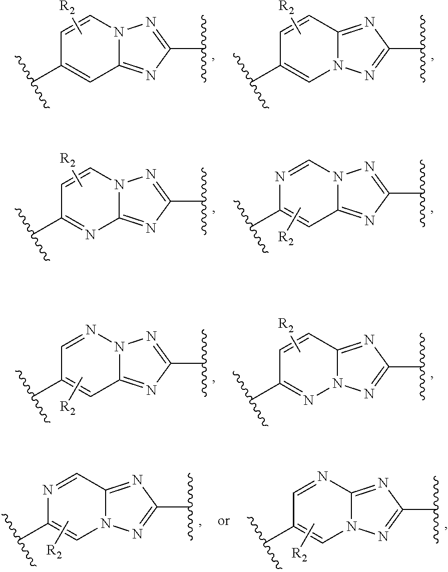

where R₂ is H, CH₃, OH, halogen, or NH₂; and wherein Rₐ is H, CH₃, or CH₂CH₃. In some embodiments, R₂ is H, CH₃, OH, halogen, or NH₂. In some embodiments, Rₐ is H, CH₃, or CH₂CH₃.

In some embodiments, the structural moiety

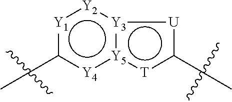

has the structure of

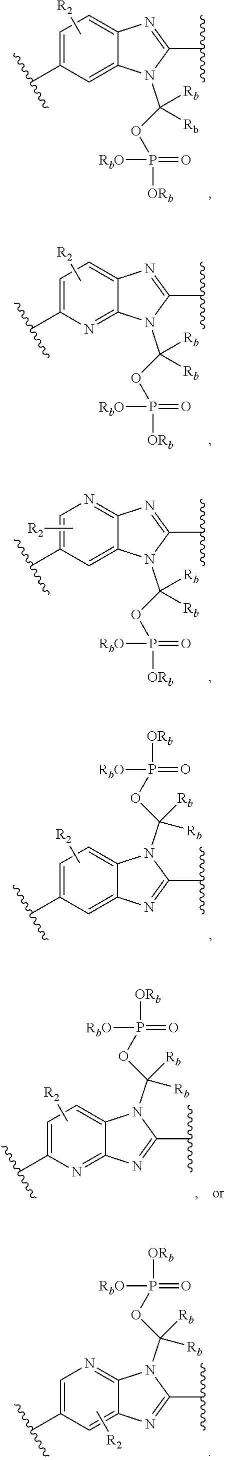

In some embodiments, the structural moiety

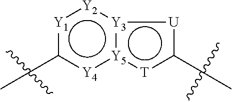

has the structure of

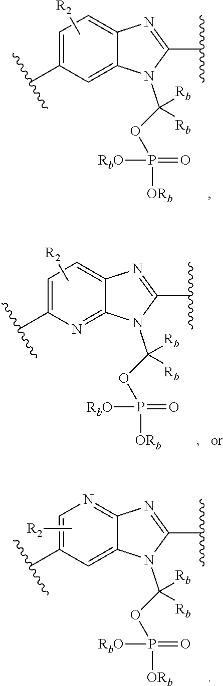

In some embodiments, the structural moiety

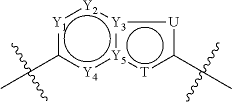

has the structure of

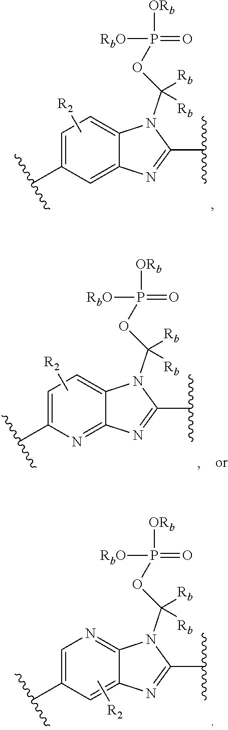

In some embodiments, each occurrence of R_{b} is independently H or CH₃.

In some embodiments, each occurrence of R₃ is independently selected from the group consisting of H, D, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₇)cycloalkyl, (C₄-C₁₀)bicycloalkyl, (C₃-C₇)heterocycloalkyl, (C₄-C₁₀)heterobicycloalkyl, aryl, heteroaryl, —ORₐ, —N(Rₐ)₂, —CORₐ, —CO₂Rₐ, CON(Rₐ)₂, —CN, —NC, NO₂, N₃, —SO₂Rₐ, —SO₂N(Rₐ)₂, and —N(Rₐ)SO₂Rₐ. In some embodiments, each occurrence of R₃ is independently (C₄-C₁₀)heterospiroalkyl, halogenated (C₃-C₇)heterocycloalkyl, aryl, or heteroaryl. In some embodiments, each occurrence of R₃ is independently selected from the group consisting of (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₇)cycloalkyl, (C₄-C₁₀)bicycloalkyl, (C₃-C₇)heterocycloalkyl, and (C₄-C₁₀)heterobicycloalkyl. In some embodiments, each occurrence of R₃ is independently selected from the group consisting of aryl and heteroaryl. In some embodiments, each occurrence of R₃ is independently selected from the group consisting of —ORₐ, —SRₐ, —N(Rₐ)₂, —CORₐ, —CO₂Rₐ, CON(Rₐ)₂, —CN, —NC, NO₂, N₃, —SO₂Rₐ, —SO₂N(Rₐ)₂, and —N(Rₐ)SO₂Rₐ. In some embodiments, each occurrence of R₃ is independently selected from the group consisting of

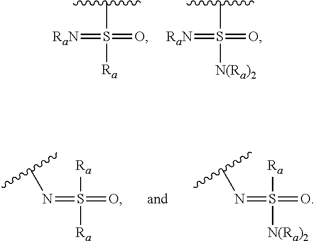

In some embodiments, each occurrence of R₃ is independently H, D, halogen, ORₐ, N(Rₐ)₂, (C₁-C₆)alkyl, (C₃-C₇)heterocycloalkyl, (C₁-C₆)alkynyl, aryl, (C₄-C₁₀)bicycloalkyl, —CN, —NC, N₃, NO₂, CORₐ, CO₂Rₐ, CON(Rₐ)₂, —SO₂Rₐ, or —SO₂N(Rₐ)₂. In some embodiments, each occurrence of R₃ is independently H, D, halogen, (C₁-C₆)alkyl, (C₃-C₇)heterocycloalkyl, N(Rₐ)₂, or —CN. In some embodiments, each occurrence of R₃ is independently H, (C₁-C₆)alkyl, (C₁-C₆)alkynyl, aryl, (C₄-C₁₀)bicycloalkyl, —SO₂Rₐ, or —SO₂N(Rₐ)₂. In some embodiments, each occurrence of R₃ is independently H, D, F, Cl, Br, CH₃, OCH₃, NH₂, N(CH₃)₂,

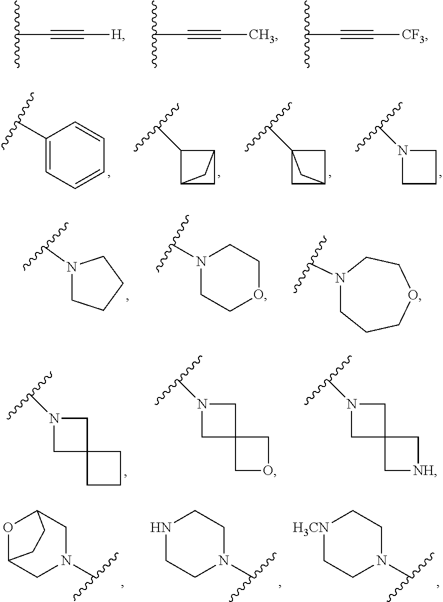

—CN, —NC, N₃, NO₂,

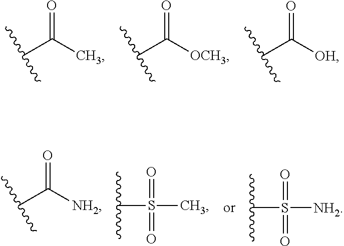

In some embodiments, each occurrence of R₃ is independently H, D, F, CH₃, N(CH₃)₂,

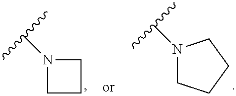 [SMILES: *N1CCC1 ; *N1CCCC1]

In some embodiments, at least one occurrence of R₃ is H, D, or halogen. In some embodiments, at least one occurrence of R₃ is H. In some embodiments, at least one occurrence of R₃ is D. In some embodiments, at least one occurrence of R₃ is F. In some embodiments, at least one occurrence of R₃ is CH₃. In some embodiments, at least one occurrence of R₃ is OCH₃. In some embodiments, at least one occurrence of R₃ is NH₂. In some embodiments, at least one occurrence of R₃ is N(CH₃)₂. In some embodiments, at least one occurrence of R₃ is

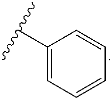 [SMILES: *c1ccccc1]

In some embodiments, at least one occurrence of R₃ is

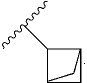 [SMILES: *C1CCC1]

In some embodiments, at least one occurrence of R₃ is

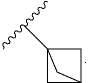 [SMILES: *C12CC(C1)C2]

In some embodiments, at least one occurrence of R₃ is

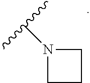 [SMILES: *N1CCC1]

In some embodiments, at least one occurrence of R₃ is

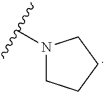 [SMILES: *N1CCCC1]

In some embodiments, at least one occurrence of R₃ is

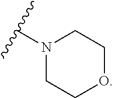 [SMILES: *N1CCOCC1]

In some embodiments, at least one occurrence of R₃ is

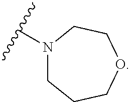 [SMILES: *N1CCCOCC1]

In some embodiments, at least one occurrence of R₃ is

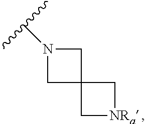

where Rₐ′ is H or (C₁-C₆)alkyl.
In some embodiments, at least one occurrence of R₃ is

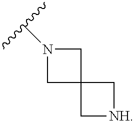 [SMILES: *N1CC2(CNC2)C1]

In some embodiments, at least one occurrence of R₃ is

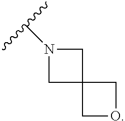 [SMILES: *N1CC2(COC2)C1]

In some embodiments, at least one occurrence of R₃ is

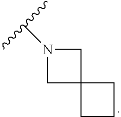 [SMILES: *N1CC2(CCC2)C1]

In some embodiments, at least one occurrence of R₃ is

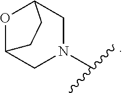 [SMILES: *N1CC2CCC(C1)O2]

In some embodiments, at least one occurrence of R₃ is

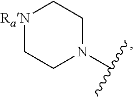

where Rₐ′ is H or (C₁-C₆)alkyl. In some embodiments, at least one occurrence of R₃ is

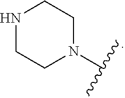 [SMILES: *N1CCNCC1]

In some embodiments, at least one occurrence of R₃ is

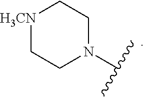

In some embodiments, at least one occurrence of R₃ is

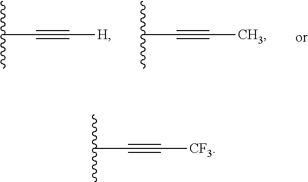

In some embodiments, at least one occurrence of R₃ is

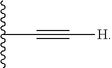

In some embodiments, at least one occurrence of R₃ is

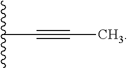 [SMILES: *C#CC]

In some embodiments, at least one occurrence of R₃ is

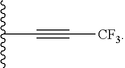 [SMILES: *C#CC(F)(F)F]

In some embodiments, at least one occurrence of R₃ is —CN. In some embodiments, at least one occurrence of R₃ is

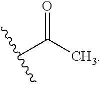 [SMILES: *C(C)=O]

In some embodiments, at least one occurrence of R₃ is

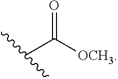 [SMILES: *C(=O)OC]

In some embodiments, at least one occurrence of R₃ is

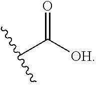 [SMILES: *C(=O)O]

In some embodiments, at least one occurrence of R₃ is —NC. In some embodiments, at least one occurrence of R₃ is

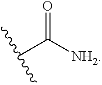 [SMILES: *C(N)=O]

In some embodiments, at least one occurrence of R₃ is NO₂. In some embodiments, at least one occurrence of R₃ is N₃. In some embodiments, at least one occurrence of R₃ is

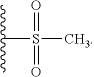 [SMILES: *S(C)(=O)=O]

In some embodiments, at least one occurrence of R₃ is

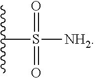 [SMILES: *S(N)(=O)=O]

In some embodiments, each occurrence of R₃ is independently selected from the group consisting of H, halogen, (C₁-C₆)alkyl, (C₁-C₆)haloalkyl, —N(Rₐ)₂, NO₂, and —ORₐ. In some embodiments, at least one occurrence of R₃ is H, CH₃, OH, NH₂, or halogen. In some embodiments, at least one occurrence of R₃ is H or CH₃. In some embodiments, at least one occurrence of R₃ is OH or NH₂. In some embodiments, at least one occurrence of R₃ is halogen. In some embodiments, at least one occurrence of R₃ is H. In some embodiments, at least one occurrence of R₃ is CF₃. In some embodiments, R₃ is H or CH₃.

In some embodiments, V is absent, O, or NRₐ. In some embodiments, V is absent. In some embodiments, V is O. In some embodiments, V is NRₐ. In some embodiments, V is NH. In some embodiments, V is NCH₃ or NCH₂CH₃.

In some embodiments, V is N(C—O)Rₐ or NSO₂Rₐ. In some embodiments, V is N(C═O)H. In some embodiments, V is N(C═O)CH₃ or N(C═O)CH₂CH₃. In some embodiments, V is NSO₂H. In some embodiments, V is NSO₂CH₃ or NSO₂CH₂CH₃.

In some embodiments, the structural moiety

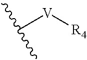

has the structure of

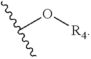

In some embodiments, the structural moiety

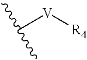

has the structure of

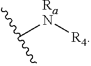

In some embodiments, the structural moiety

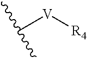

has the structure of

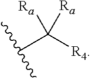

In some embodiments, the V and R₄ of the structural moiety

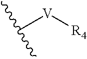

taken together form a (C₄-C₁₀)heterospiroalkyl.

In some embodiments, R₄ is selected from the group consisting of (C₁-C₆)alkyl, (C₃-C₇)cycloalkyl, (C₄-C₁₀)bicycloalkyl, (C₃-C₇)heterocycloalkyl, (C₄-C₁₀)heterobicycloalkyl, aryl, and heteroaryl, each optionally substituted with one or more R₅. In some embodiments, R₄ is substituted by 0, 1, 2, 3, 4, 5 or 6 R₅ substituents, wherein each R₅ is independently selected from the group consisting of H, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₇)cycloalkyl, (C₄-C₁₀)bicycloalkyl, (C₃-C₇)heterocycloalkyl, (C₄-C₁₀)heterobicycloalkyl, aryl, heteroaryl, —ORₐ, —SRₐ, —N(Rₐ)₂, N(Rₐ)CORₐ, —CORₐ, —CO₂Rₐ, CON(Rₐ)₂, —CN, —NC, NO₂, N₃, —SO₂Rₐ, —SO₂N(Rₐ)₂, —N(Rₐ)SO₂Rₐ,

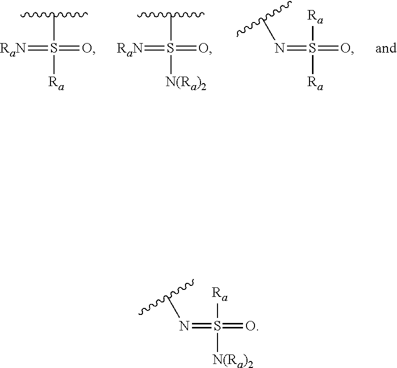

In some embodiments, each occurrence of R₅ is independently (C₄-C₁₀)heterospiroalkyl, halogenated (C₃-C₇)heterocycloalkyl, aryl, or heteroaryl. In some embodiments, R₄ is

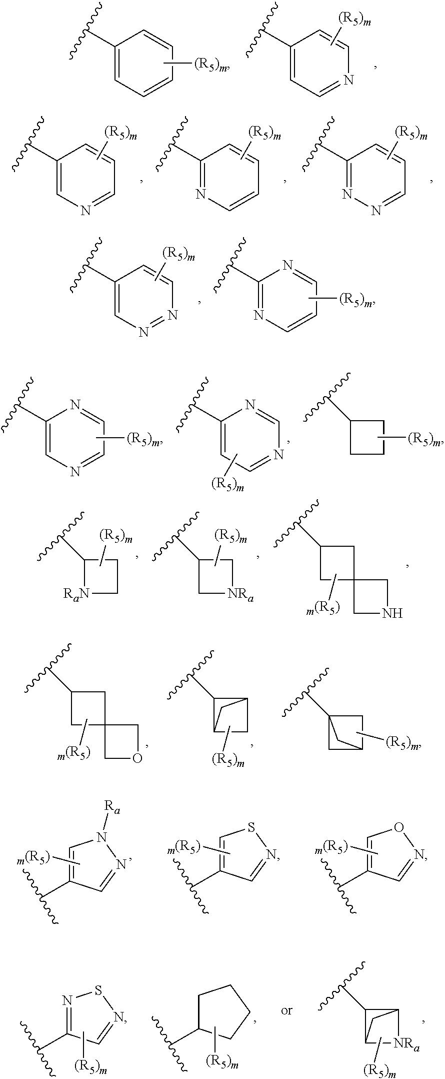

where in

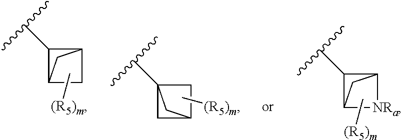

R₅ may be attached to any position of the bicycloalkyl or heterobicycloalkyl including the bridge head carbon, and where in

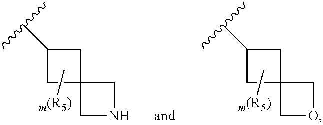

R₅ may be attached to any available position in either ring. In some embodiments, R₄ is

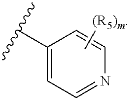

In some embodiments, R₄ is

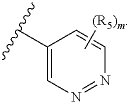

In some embodiments, R₄ is

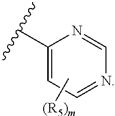

In some embodiments, R₄ is

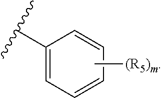

In some embodiments, R₄ is

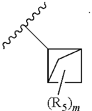

In some embodiments, R₄ is

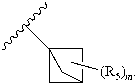

In some embodiments, R₄ is

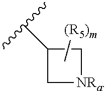

In some embodiments, R₄ is

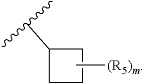

In some embodiments, m is an integer from 0-3. In some embodiments, m is 0. In some embodiments, m is 1. In some embodiments, m is 2. In some embodiments, m is 3.

In some embodiments, the structural moiety

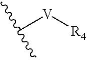

has the structure of

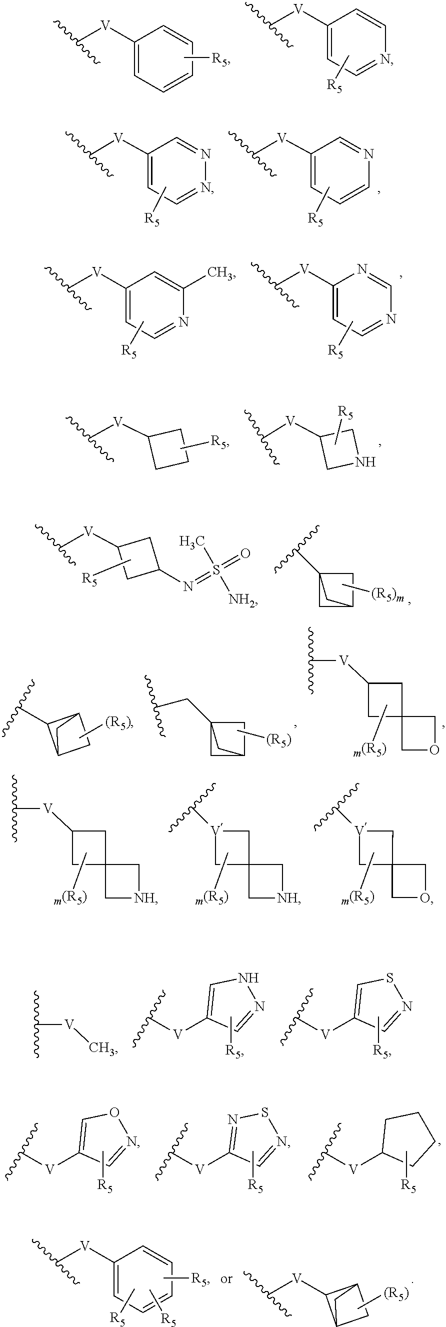

In some embodiments, V is C(Rₐ)₂, O, NRₐ, N(C═O)Rₐ, or NSO₂Rₐ. In some embodiments, V is CRₐ or N.

In some embodiments, each occurrence of R₅ is independently selected from the group consisting of H, D, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₇)cycloalkyl, (C₄-C₁₀)bicycloalkyl, (C₃-C₇)heterocycloalkyl, (C₄-C₁₀)heterobicycloalkyl, aryl, heteroaryl, —ORₐ, —N(Rₐ)₂, —CORₐ, —CO₂Rₐ, N(Rₐ)CORₐ, CON(Rₐ)₂, —CN, —NC, NO₂, N₃, —SO₂Rₐ, —SO₂N(Rₐ)₂, and —N(Rₐ)SO₂Rₐ. In some embodiments, each occurrence of R₅ is independently selected from the group consisting of (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₇)cycloalkyl, (C₄-C₁₀)bicycloalkyl, (C₃-C₇)heterocycloalkyl, and (C₄-C₁₀)heterobicycloalkyl. In some embodiments, each occurrence of R₅ is independently selected from the group consisting of (C₄-C₁₀)heterospiroalkyl, halogenated (C₃-C₇)heterocycloalkyl, aryl and heteroaryl. In some embodiments, each occurrence of R₅ is independently selected from the group consisting of ORₐ, —SRₐ, —N(Rₐ)₂, —CORₐ, —CO₂Rₐ, CON(Rₐ)₂, —CN, —NC, NO₂, N₃, —SO₂Rₐ, N(Rₐ)CORₐ, —SO₂N(Rₐ)₂, and —N(Rₐ)SO₂Rₐ. In some embodiments, each occurrence of R₅ is independently selected from the group consisting of

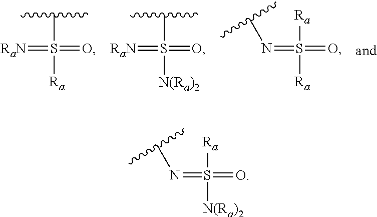

In some embodiments, each occurrence of R₅ is independently H, D, halogen, ORₐ, N(Rₐ)₂, (C₁-C₆)alkyl, (C₃-C₇)heterocycloalkyl, (C₁-C₆)alkynyl, aryl, (C₄-C₁₀)bicycloalkyl, —CN, —NC, N₃, NO₂, CORₐ, CO₂Rₐ, CON(Rₐ)₂, —SO₂Rₐ, N(Rₐ) CORₐ, or —SO₂N(Rₐ)₂. In some embodiments, each occurrence of R₅ is independently H, D, halogen, (C₁-C₆)alkyl, (C₃-C₇)heterocycloalkyl, N(Rₐ)CORₐ, N(Rₐ)₂, or —CN. In some embodiments, each occurrence of R₅ is independently H, (C₁-C₆)alkyl, (C₁-C₆)alkynyl, aryl, (C₄-C₁₀)bicycloalkyl, —SO₂Rₐ, or —SO₂N(Rₐ)₂. In some embodiments, each occurrence of R₅ is independently H, D, F, Cl, Br, CH₃, CF₃, OCH₃, NH₂, N(CH₃)₂,

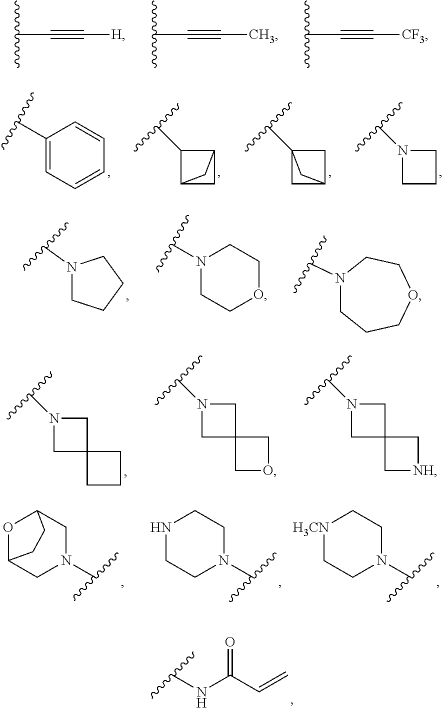

—CN, —NC, N₃, NO₂,

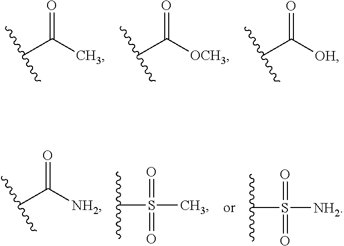

In some embodiments, each occurrence of R₅ is independently H, D, F, CH₃, N(CH₃)₂,

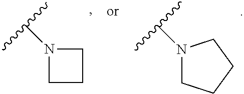 [SMILES: *N1CCC1 ; *N1CCCC1]

In some embodiments, at least one occurrence of R₅ is H, D, or halogen. In some embodiments, at least one occurrence of R₅ is H. In some embodiments, at least one occurrence of R₅ is D. In some embodiments, at least one occurrence of R₅ is F. In some embodiments, at least one occurrence of R₅ is CH₃. In some embodiments, at least one occurrence of R₅ is OCH₃. In some embodiments, at least one occurrence of R₅ is NH₂. In some embodiments, at least one occurrence of R₅ is N(CH₃)₂. In some embodiments, at least one occurrence of R₅ is

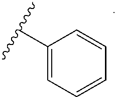 [SMILES: *c1ccccc1]

In some embodiments, at least one occurrence of R₅ is

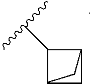 [SMILES: *C1CCC1]

In some embodiments, at least one occurrence of R₅ is

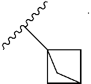 [SMILES: *C12CC(C1)C2]

In some embodiments, at least one occurrence of R₅ is

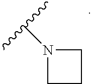 [SMILES: *N1CCC1]

In some embodiments, at least one occurrence of R₅ is

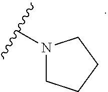 [SMILES: *N1CCCC1]

In some embodiments, at least one occurrence of R₅ is

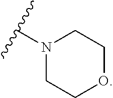 [SMILES: *N1CCOCC1]

In some embodiments, at least one occurrence of R₅ is

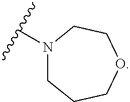 [SMILES: *N1CCCOCC1]

In some embodiments, at least one occurrence of R₅ is

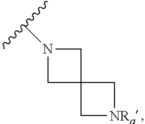

where Rₐ′ is H or (C₁-C₆)alkyl. In some embodiments, at least one occurrence of R₅ is

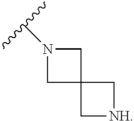 [SMILES: *N1CC2(CNC2)C1]

In some embodiments, at least one occurrence of R₅ is

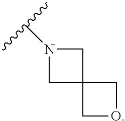 [SMILES: *N1CC2(COC2)C1]

In some embodiments, at least one occurrence of R₅ is

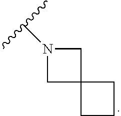 [SMILES: *N1CC2(CCC2)C1]

In some embodiments, at least one occurrence of R₅ is

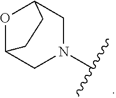 [SMILES: *N1CC2CCC(C1)O2]

In some embodiments, at least one occurrence of R₅ is

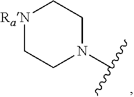

where Rₐ′ is H or (C₁-C₆)alkyl. In some embodiments, at least one occurrence of R₅ is

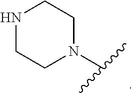 [SMILES: *N1CCNCC1]

In some embodiments, at least one occurrence of R₅ is

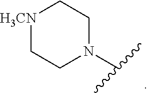

In some embodiments, at least one occurrence of R₅ is

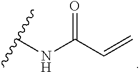 [SMILES: *NC(=O)C=C]

In some embodiments, at least one occurrence of R₅ is

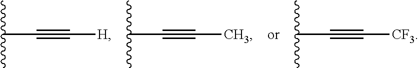

In some embodiments, at least one occurrence of R₅ is

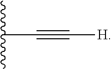

In some embodiments, at least one occurrence of R₅ is

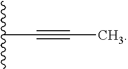 [SMILES: *C#CC]

In some embodiments, at least one occurrence of R₅ is

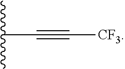 [SMILES: *C#CC(F)(F)F]

In some embodiments, at least one occurrence of R₅ is —CN. In some embodiments, at least one occurrence of R₅ is —NC. In some embodiments, at least one occurrence of R₅ is

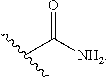 [SMILES: *C(N)=O]

In some embodiments, at least one occurrence of R₅ is

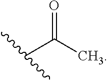 [SMILES: *C(C)=O]

In some embodiments, at least one occurrence of R₅ is

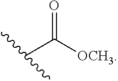 [SMILES: *C(=O)OC]

In some embodiments, at least one occurrence of R₅ is

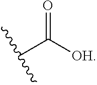 [SMILES: *C(=O)O]

In some embodiments, at least one occurrence of R₅ is NO₂. In some embodiments, at least one occurrence of R₅ is N₃. In some embodiments, at least one occurrence of R₅ is

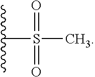 [SMILES: *S(C)(=O)=O]

In some embodiments, at least one occurrence of R₅ is

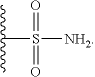 [SMILES: *S(N)(=O)=O]

In some embodiments, each occurrence of R₅ is independently selected from the group consisting of halogen, (C₁-C₆)alkyl, (C₁-C₆)haloalkyl, ORₐ, —N(Rₐ)₂, —CORₐ, —CO₂Rₐ, CON(Rₐ)₂, N(Rₐ)CORₐ, —CN, NO₂, —SO₂Rₐ, —SO₂N(Rₐ)₂, —N(Rₐ)SO₂Rₐ,

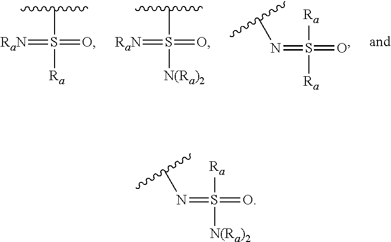

In some embodiments, at least one occurrence of R₅ is (C₁-C₆)alkyl, halogen, OH, NH₂, or

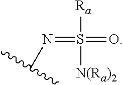

In some embodiments, at least one occurrence of R₅ is CH₃, halogen, OH, or NH₂. In some embodiments, at least one occurrence of R₅ is OH. In some embodiments, at least one occurrence of R₅ is CH₃. In some embodiments, at least one occurrence of R₅ is

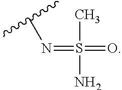 [SMILES: *N=S(C)(N)=O]

In any one of embodiments described herein, each occurrence of Rₐ is H, (C₁-C₆)alkyl, (C₂-C₆)alkenyl, (C₃-C₇)cycloalkyl, aryl, or heteroaryl. In any one of embodiments described herein, at least one occurrence of Rₐ is aryl, or heteroaryl. In any one of embodiments described herein, each occurrence of Rₐ is independently H, (C₁-C₆)alkyl, (C₂-C₆)alkenyl, or (C₃-C₇)cycloalkyl, or two Rₐ taken together form a 5- or 6-membered ring optionally substituted with halogen or (C₁-C₆)alkyl. In some embodiments, each occurrence of Rₐ is independently H or (C₁-C₆)alkyl. In some embodiments, each occurrence of Rₐ is independently (C₂-C₆)alkenyl. In some embodiments, each occurrence of Rₐ is independently H, CH₃, or CH₂CH₃. In some embodiments, at least one occurrence of Rₐ is H or CH₃. In some embodiments, each occurrence of Rₐ is H. In some embodiments, each occurrence of Rₐ is CH₃. In some embodiments, at least one occurrence of Rₐ is (C₃-C₇)cycloalkyl, optionally substituted with halogen or (C₁-C₆)alkyl. In some embodiments, at least one occurrence of Rₐ is cyclopropyl, cyclobutyl, cyclopentyl, or cyclohexyl, optionally substituted with halogen or (C₁-C₆)alkyl.

In some embodiments, the structural moiety

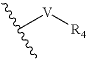

has the structure of

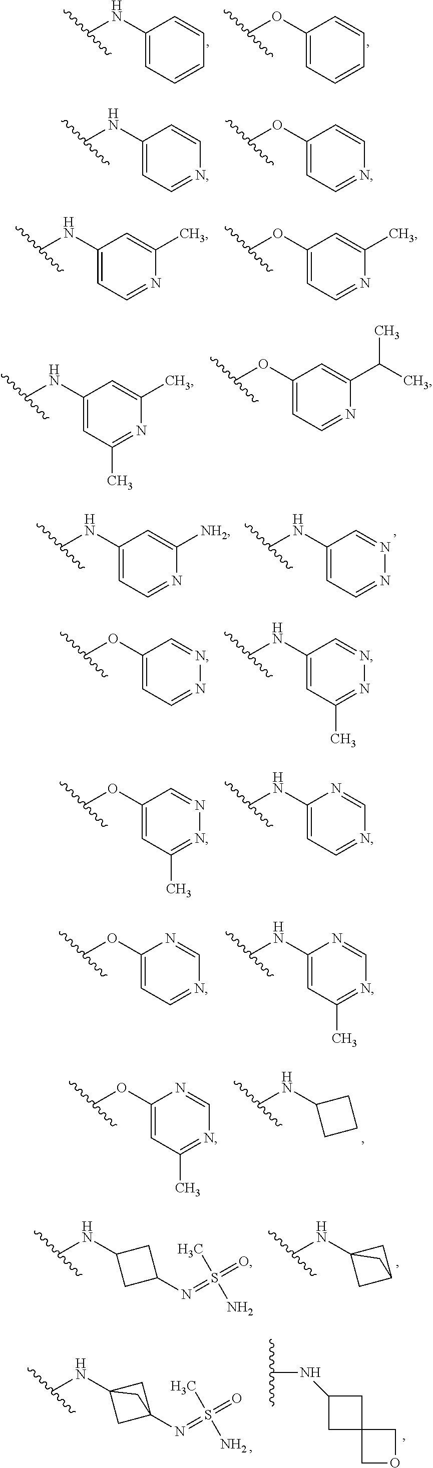

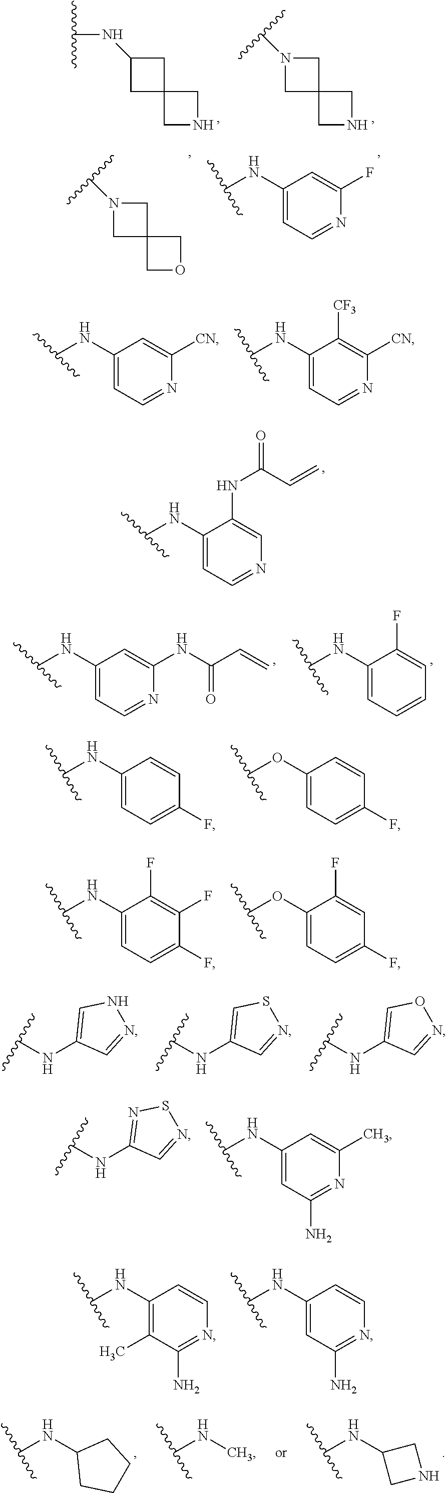

In some embodiments, the structural moiety

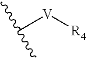

has the structure of

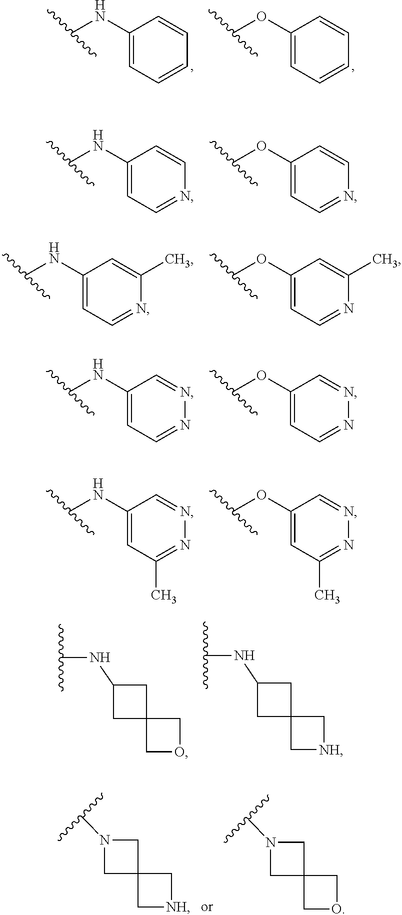

In some embodiments, the structural moiety

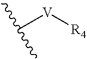

has the structure of

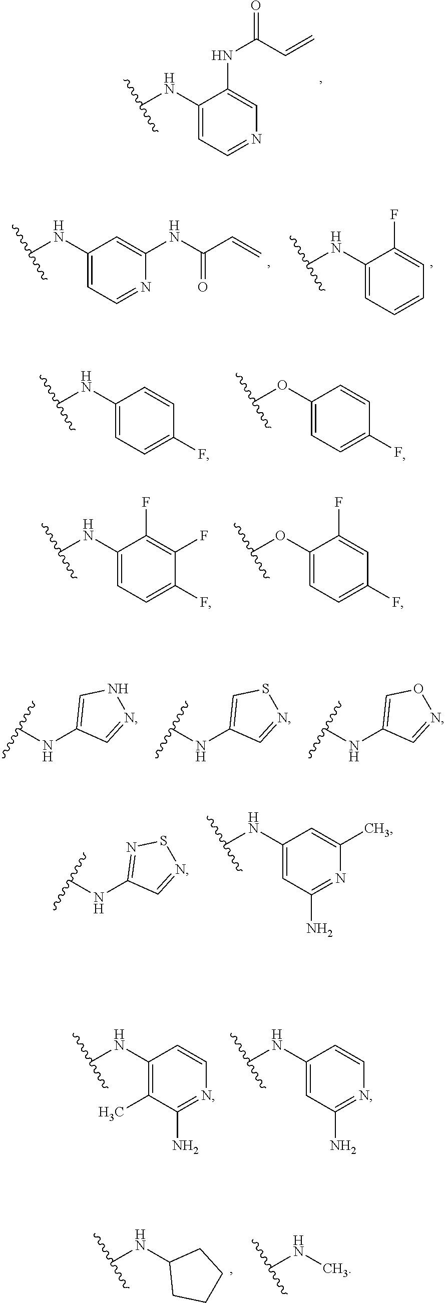

In some embodiments, the compound of Formula Ia has the structure of

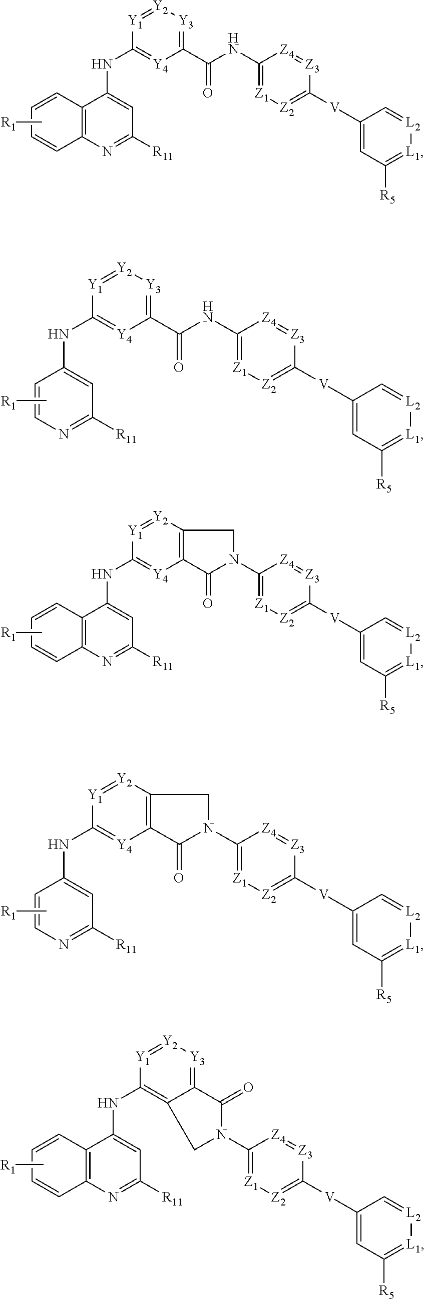

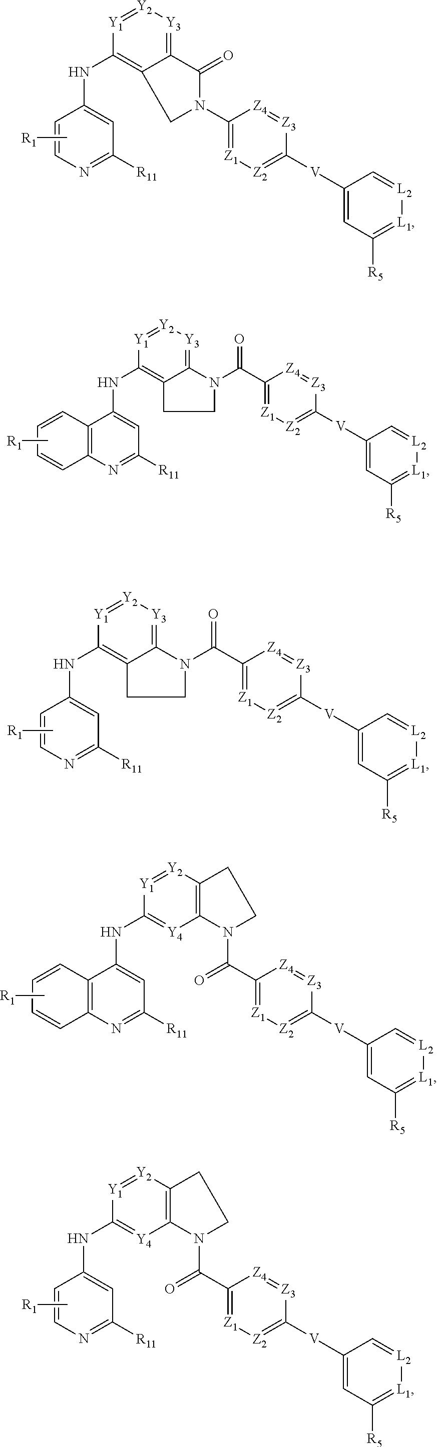

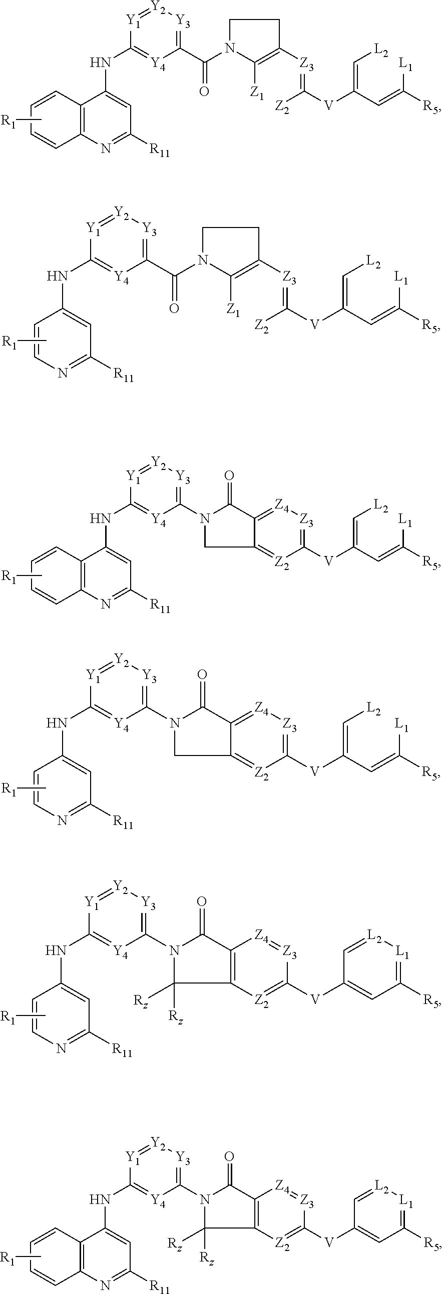

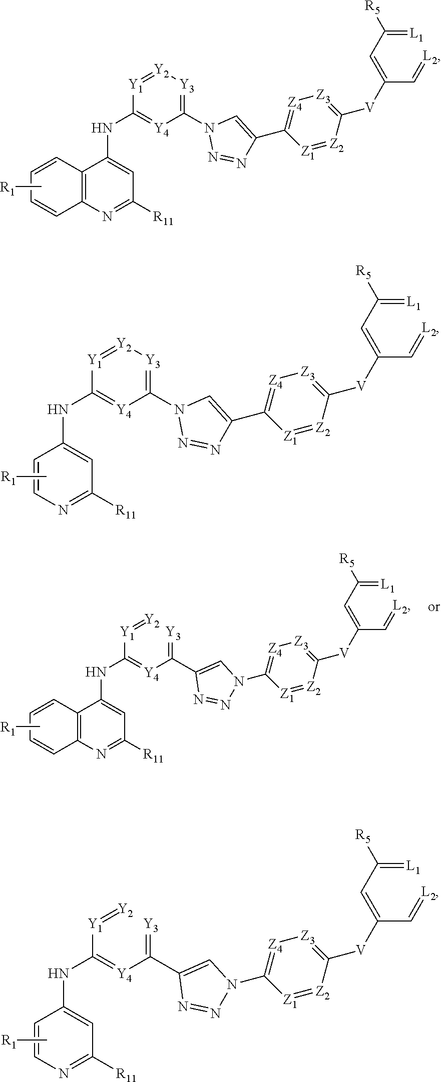

where R₁ is H, (C₁-C₆)alkyl, N(Rₐ)₂, (C₃-C₇)heterocycloalkyl, or halogen; R₅ and R₁₁ are each independently H or CH₃; Y₁, Y₂, Y₃, Y₄, Z₁, Z₂, Z₃, Z₄, L₁, and L₂ are each independently CH or N; and V is NH or O. In some embodiments, the compound of Formula Ia has the structure of

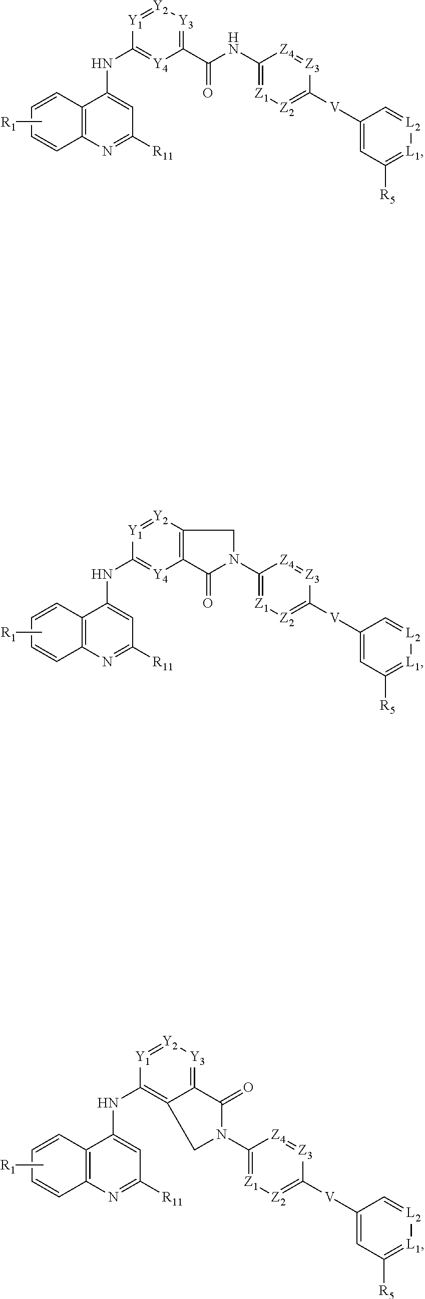

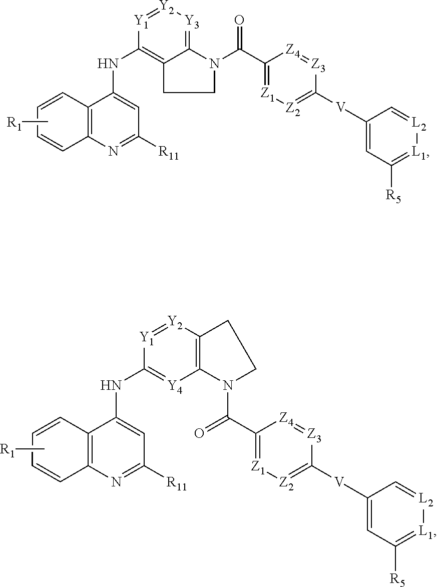

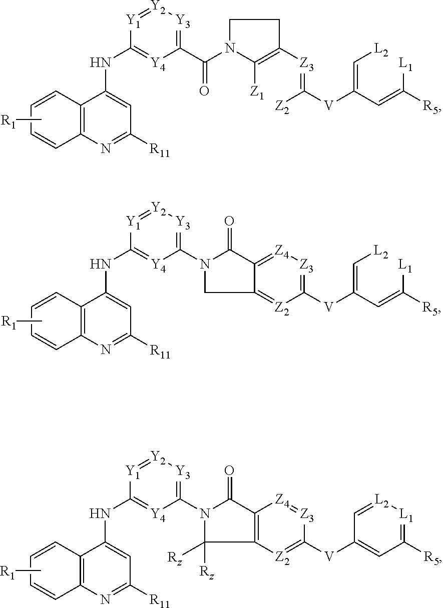

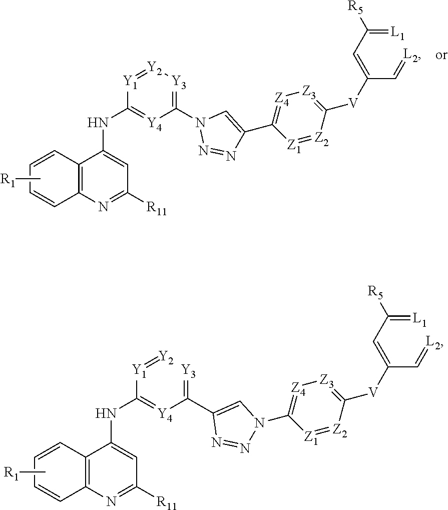

where R₁ is H, (C₁-C₆)alkyl, N(Rₐ)₂, (C₃-C₇)heterocycloalkyl, or halogen; R₅ and R₁₁ are each independently H or CH₃; Y₁, Y₂, Y₃, Y₄, Z₁, Z₂, Z₃, Z₄, L₁, and L₂ are each independently CH or N; and V is NH or O. In some embodiments, the compound of Formula Ia has the structure of

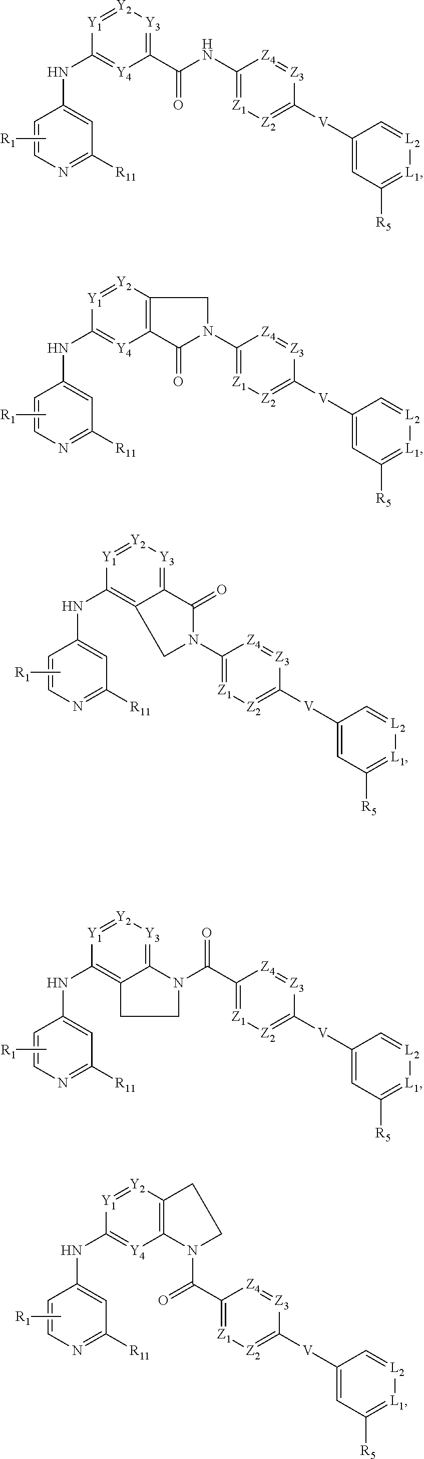

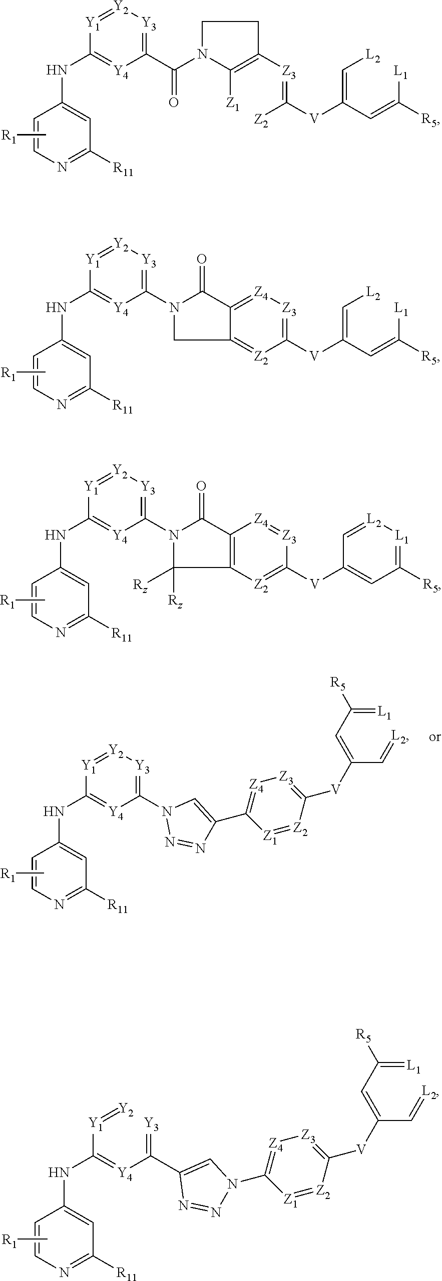

where R₁ is H, (C₁-C₆)alkyl, N(Rₐ)₂, (C₃-C₇)heterocycloalkyl, or halogen; R₅ and R₁₁ are each independently H or CH₃; Y₁, Y₂, Y₃, Y₄, Z₁, Z₂, Z₃, Z₄, L₁, and L₂ are each independently CH or N; and V is NH or O. In some embodiments, R₁ is H, F, Cl, Br, CH₃, CH₂CH₃, or CH(CH₃)₂,

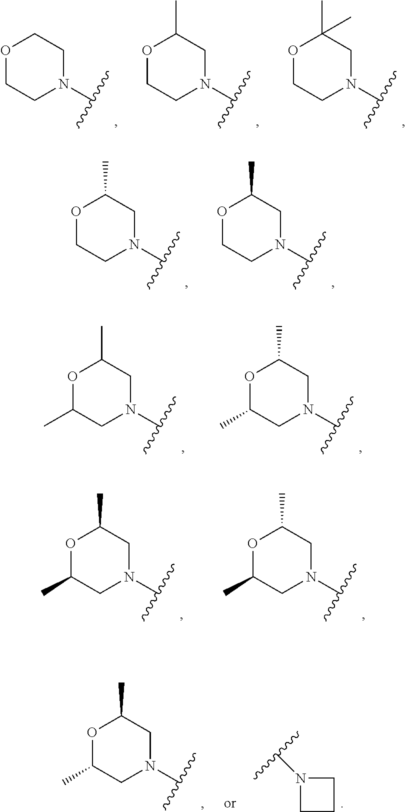

In some embodiments, R₁ is

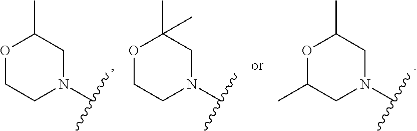

In some embodiments, R₁ is

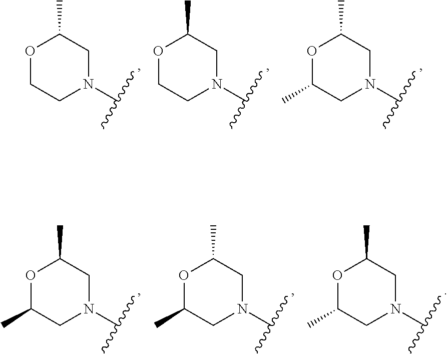

In some embodiments, the compound of Formula Ib has the structure of

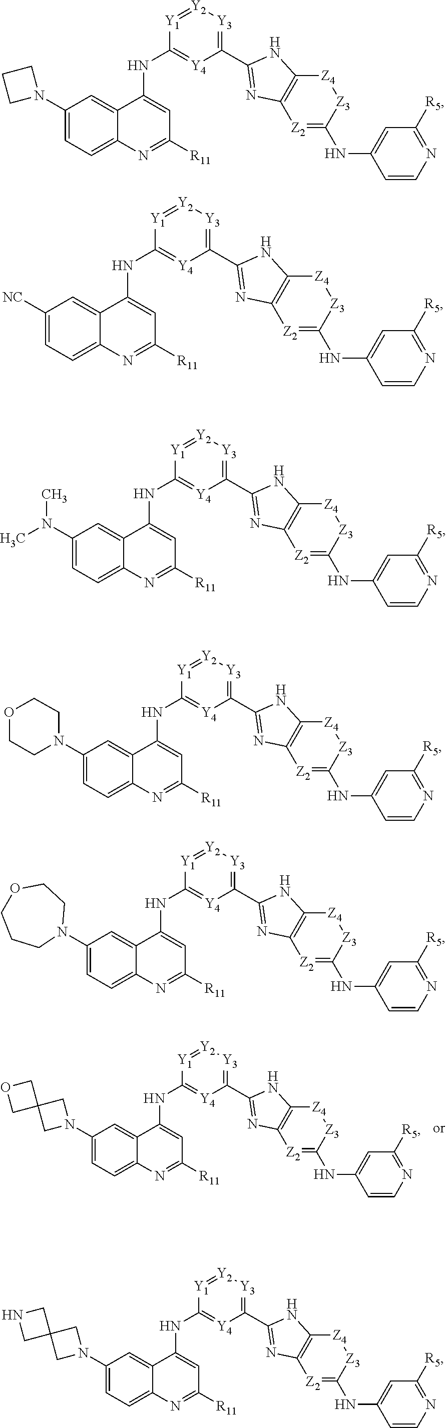

where R₁₁ and R₅ are each independently H or CH₃; and Y₁, Y₂, Y₃, Y₄, Z₂, Z₃, and Z₄ are each independently CH or N. In some embodiments, the compound of Formula Ib has the structure of

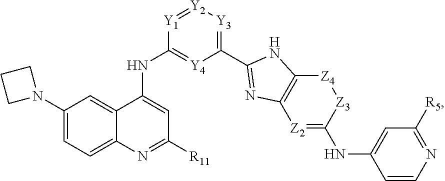

where R₁₁ and R₅ are each independently H or CH₃; and Y₁, Y₂, Y₃, Y₄, Z₂, Z₃, and Z₄ are each independently CH or N. In some embodiments, the compound of Formula Ib has the structure of

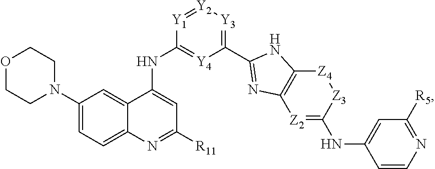

where R₁₁ and R₅ are each independently H or CH₃; and Y₁, Y₂, Y₃, Y₄, Z₂, Z₃, and Z₄ are each independently CH or N.

In some embodiments, the compound of Formula Ib has the structure

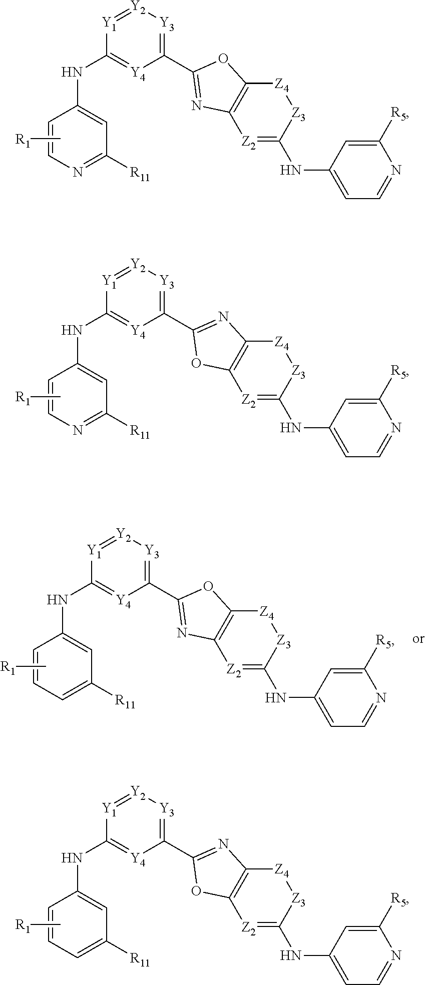

wherein R₁, R₅, and R₁₁ are each independently H, halogen, or CH₃; and Y₁, Y₂, Y₃, Y₄, Z₂, Z₃, and Z₄ are each independently CH or N.

In some embodiments, the compound of Formula Ic is

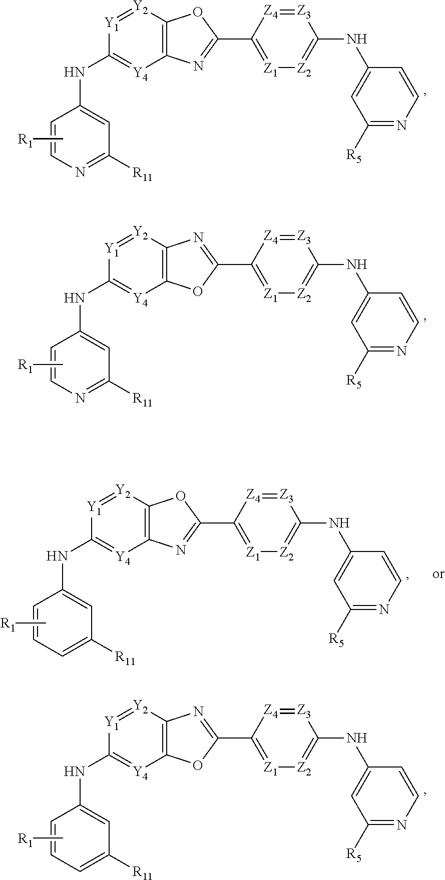

wherein R₁, R₅, and R₁₁ are each independently H, halogen, or CH₃; and Y₁, Y₂, Y₄, Z₁, Z₂, Z₃, and Z₄ are each independently CH or N.

In some embodiments, the compound of Formula Ia, Ib, or Ic activates Akt3 and is the compound

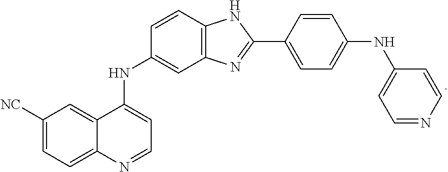 [SMILES: N#Cc1ccc2nccc(Nc3ccc4[nH]c(-c5ccc(Nc6ccncc6)cc5)nc4c3)c2c1]

In some embodiments, the compound of Formula Ia, Ib, or Ic activates Akt3 and is Compound 2 as shown in Table 2.

In some embodiments, the compound of Formula Ia, Ib, or Ic inhibits Akt3 and is selected from the group consisting of

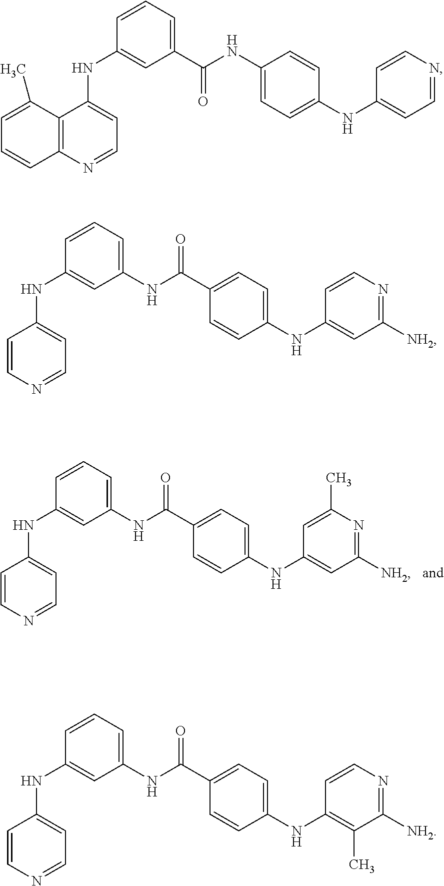 [SMILES: Cc1cccc2nccc(Nc3cccc(C(=O)Nc4ccc(Nc5ccncc5)cc4)c3)c12 ; Nc1cc(Nc2ccc(C(=O)Nc3cccc(Nc4ccncc4)c3)cc2)ccn1 ; Cc1cc(Nc2ccc(C(=O)Nc3cccc(Nc4ccncc4)c3)cc2)cc(N)n1 ; Cc1c(Nc2ccc(C(=O)Nc3cccc(Nc4ccncc4)c3)cc2)ccnc1N]

In some embodiments, the compound of Formula Ia, Ib, or Ic inhibits Akt3 and is selected from the group consisting of

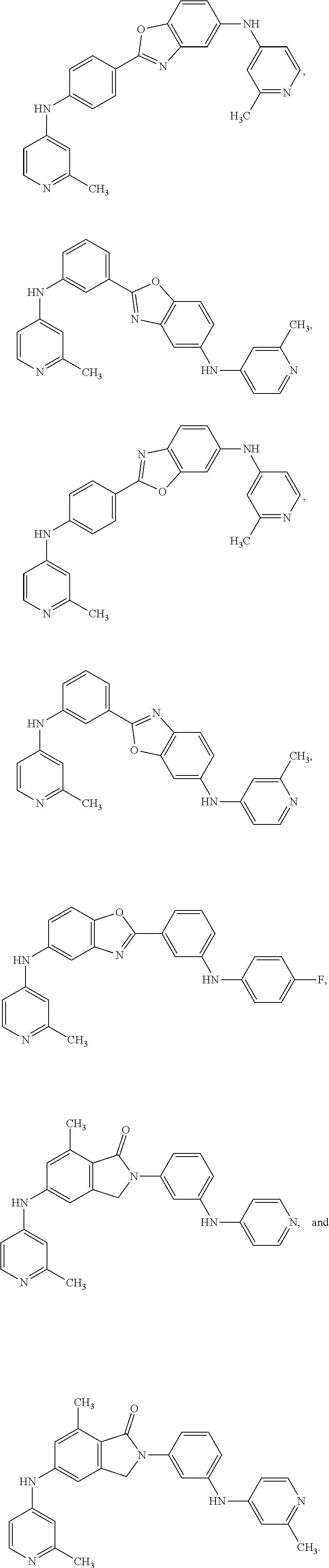

In some embodiments, the compound of Formula Ia, Ib, or Ic inhibits Akt3 and is selected from the group consisting of Compounds 3, 18-21, 26, 27, 30, 32-34, and 37-45 as shown in Table 1.

In some embodiments, the compound of Formula Ia is

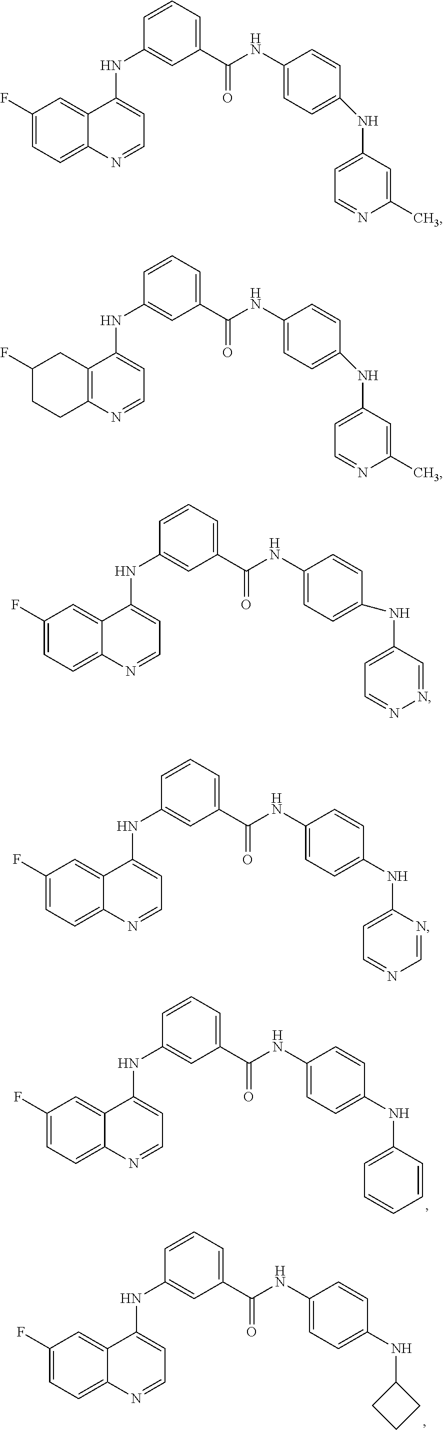 [SMILES: Cc1cc(Nc2ccc(NC(=O)c3cccc(Nc4ccnc5ccc(F)cc45)c3)cc2)ccn1 ; Cc1cc(Nc2ccc(NC(=O)c3cccc(Nc4ccnc5c4CC(F)CC5)c3)cc2)ccn1 ; O=C(Nc1ccc(Nc2ccnnc2)cc1)c1cccc(Nc2ccnc3ccc(F)cc23)c1 ; O=C(Nc1ccc(Nc2ccncn2)cc1)c1cccc(Nc2ccnc3ccc(F)cc23)c1 ; O=C(Nc1ccc(Nc2ccccc2)cc1)c1cccc(Nc2ccnc3ccc(F)cc23)c1 ; O=C(Nc1ccc(NC2CCC2)cc1)c1cccc(Nc2ccnc3ccc(F)cc23)c1]

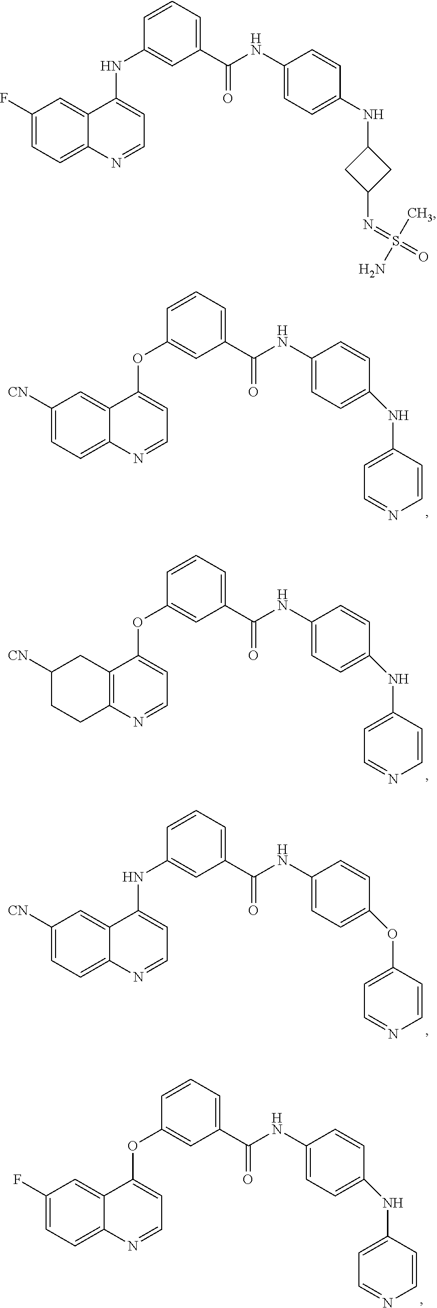

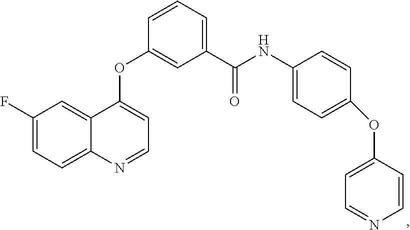 [SMILES: O=C(Nc1ccc(Oc2ccncc2)cc1)c1cccc(Oc2ccnc3ccc(F)cc23)c1]

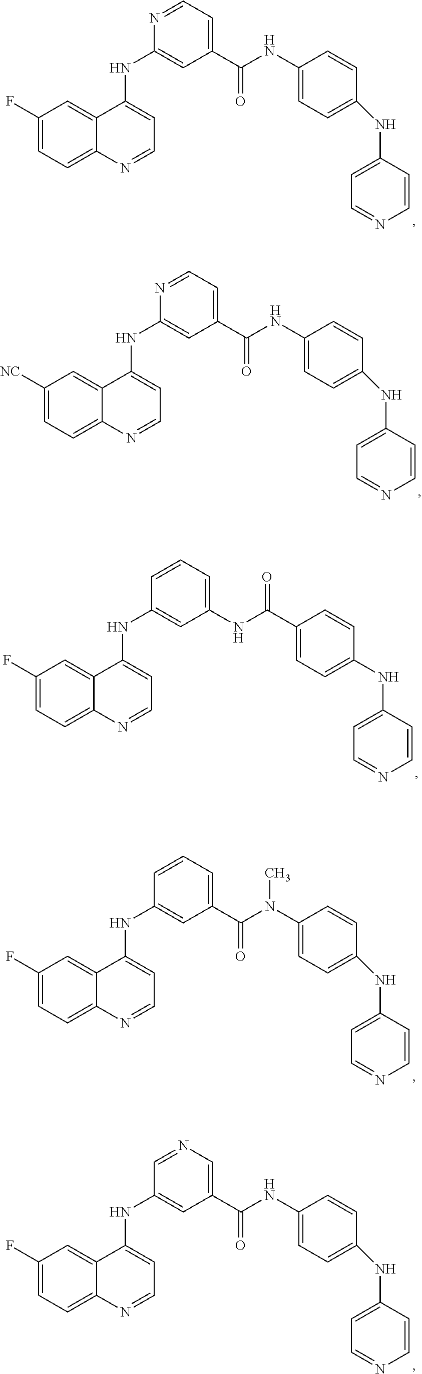 [SMILES: O=C(Nc1ccc(Nc2ccncc2)cc1)c1ccnc(Nc2ccnc3ccc(F)cc23)c1 ; N#Cc1ccc2nccc(Nc3cc(C(=O)Nc4ccc(Nc5ccncc5)cc4)ccn3)c2c1 ; O=C(Nc1cccc(Nc2ccnc3ccc(F)cc23)c1)c1ccc(Nc2ccncc2)cc1 ; CN(C(=O)c1cccc(Nc2ccnc3ccc(F)cc23)c1)c1ccc(Nc2ccncc2)cc1 ; O=C(Nc1ccc(Nc2ccncc2)cc1)c1cncc(Nc2ccnc3ccc(F)cc23)c1]

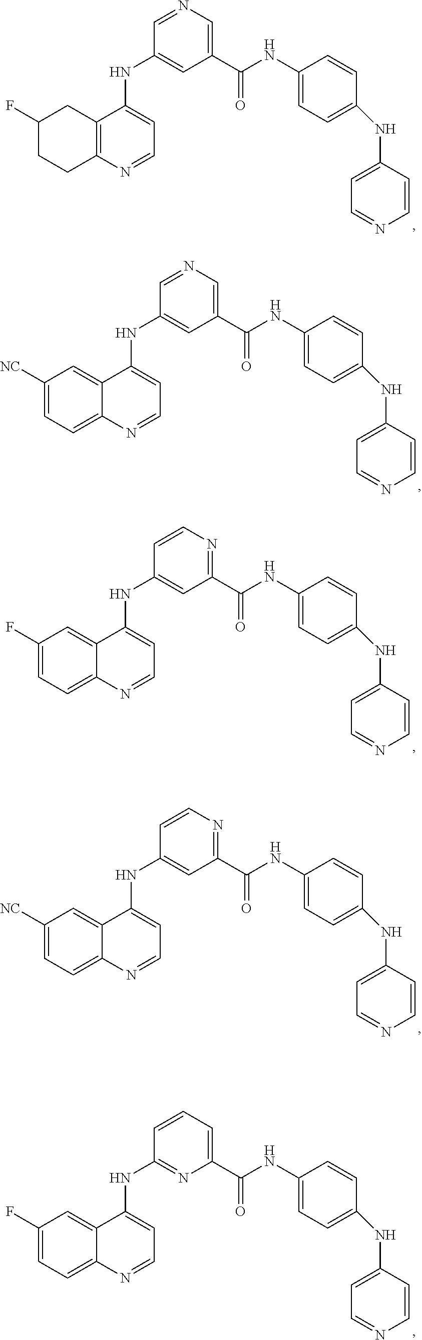 [SMILES: O=C(Nc1ccc(Nc2ccncc2)cc1)c1cncc(Nc2ccnc3c2CC(F)CC3)c1 ; N#Cc1ccc2nccc(Nc3cncc(C(=O)Nc4ccc(Nc5ccncc5)cc4)c3)c2c1 ; O=C(Nc1ccc(Nc2ccncc2)cc1)c1cc(Nc2ccnc3ccc(F)cc23)ccn1 ; N#Cc1ccc2nccc(Nc3ccnc(C(=O)Nc4ccc(Nc5ccncc5)cc4)c3)c2c1 ; O=C(Nc1ccc(Nc2ccncc2)cc1)c1cccc(Nc2ccnc3ccc(F)cc23)n1]

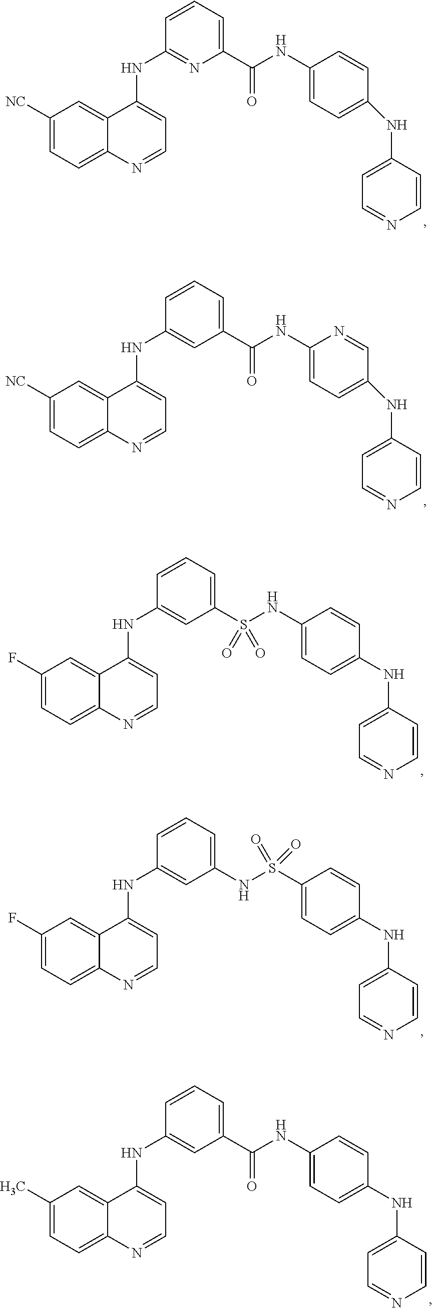 [SMILES: N#Cc1ccc2nccc(Nc3cccc(C(=O)Nc4ccc(Nc5ccncc5)cc4)n3)c2c1 ; N#Cc1ccc2nccc(Nc3cccc(C(=O)Nc4ccc(Nc5ccncc5)cn4)c3)c2c1 ; O=S(=O)(Nc1ccc(Nc2ccncc2)cc1)c1cccc(Nc2ccnc3ccc(F)cc23)c1 ; O=S(=O)(Nc1cccc(Nc2ccnc3ccc(F)cc23)c1)c1ccc(Nc2ccncc2)cc1 ; Cc1ccc2nccc(Nc3cccc(C(=O)Nc4ccc(Nc5ccncc5)cc4)c3)c2c1]

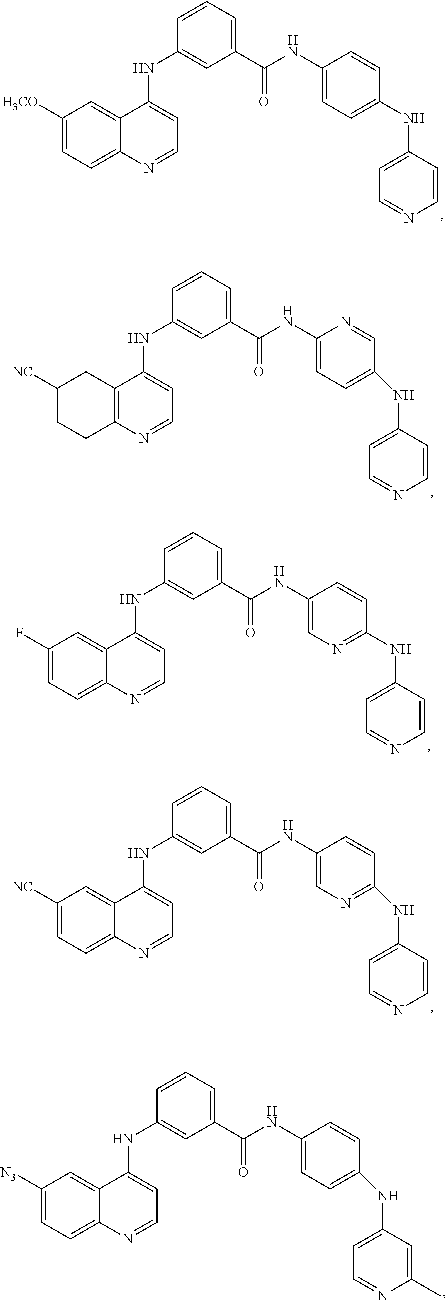 [SMILES: COc1ccc2nccc(Nc3cccc(C(=O)Nc4ccc(Nc5ccncc5)cc4)c3)c2c1 ; N#CC1CCc2nccc(Nc3cccc(C(=O)Nc4ccc(Nc5ccncc5)cn4)c3)c2C1 ; O=C(Nc1ccc(Nc2ccncc2)nc1)c1cccc(Nc2ccnc3ccc(F)cc23)c1 ; N#Cc1ccc2nccc(Nc3cccc(C(=O)Nc4ccc(Nc5ccncc5)nc4)c3)c2c1 ; Cc1cc(Nc2ccc(NC(=O)c3cccc(Nc4ccnc5ccc(N=[N+]=[N-])cc45)c3)cc2)ccn1]

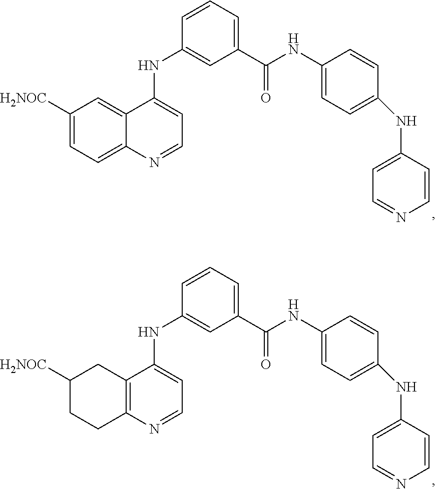 [SMILES: NC(=O)c1ccc2nccc(Nc3cccc(C(=O)Nc4ccc(Nc5ccncc5)cc4)c3)c2c1 ; NC(=O)C1CCc2nccc(Nc3cccc(C(=O)Nc4ccc(Nc5ccncc5)cc4)c3)c2C1]

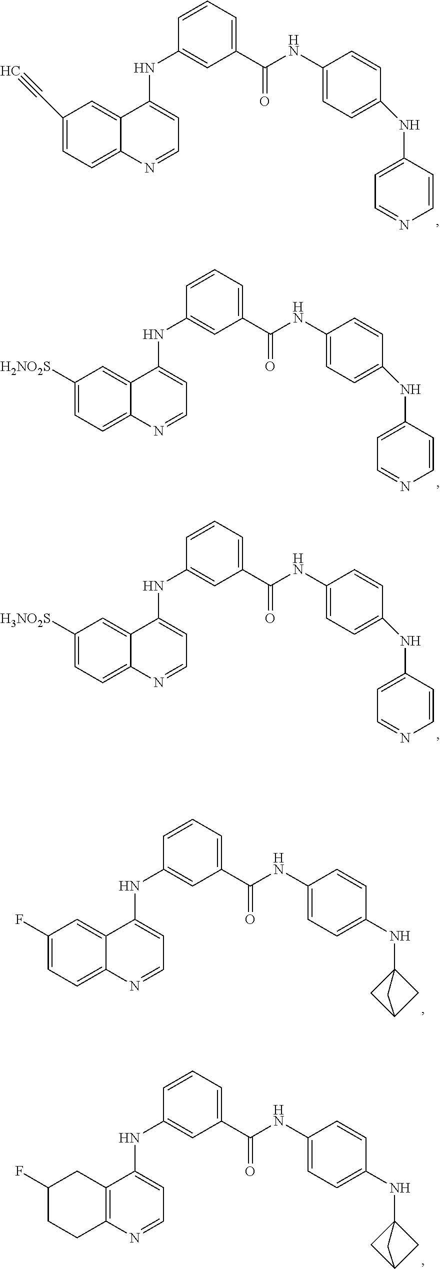

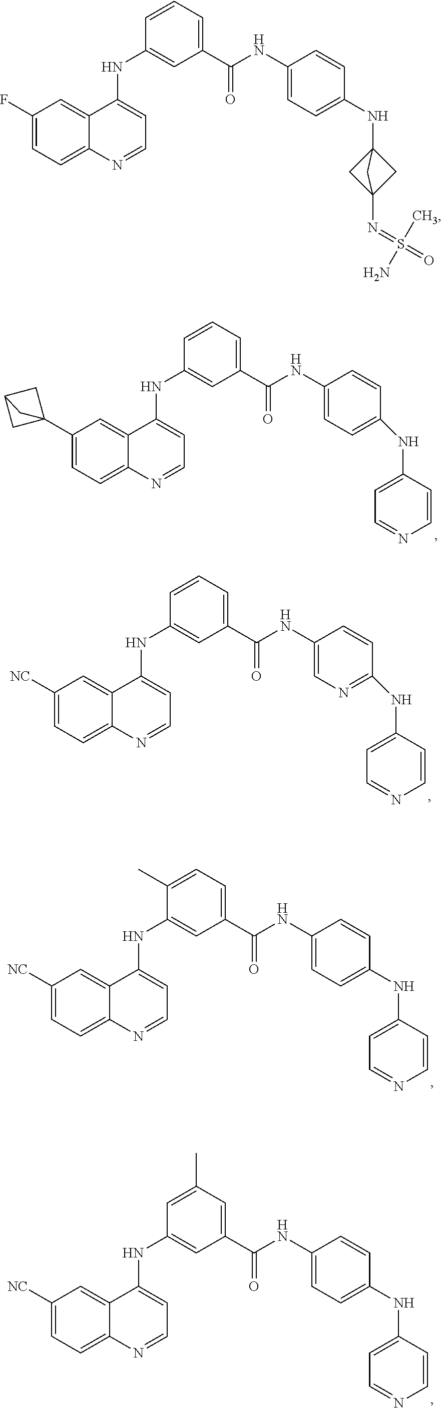 [SMILES: CS(N)(=O)=NC12CC(Nc3ccc(NC(=O)c4cccc(Nc5ccnc6ccc(F)cc56)c4)cc3)(C1)C2 ; O=C(Nc1ccc(Nc2ccncc2)cc1)c1cccc(Nc2ccnc3ccc(C45CC(C4)C5)cc23)c1 ; N#Cc1ccc2nccc(Nc3cccc(C(=O)Nc4ccc(Nc5ccncc5)nc4)c3)c2c1 ; Cc1ccc(C(=O)Nc2ccc(Nc3ccncc3)cc2)cc1Nc1ccnc2ccc(C#N)cc12 ; Cc1cc(Nc2ccnc3ccc(C#N)cc23)cc(C(=O)Nc2ccc(Nc3ccncc3)cc2)c1]

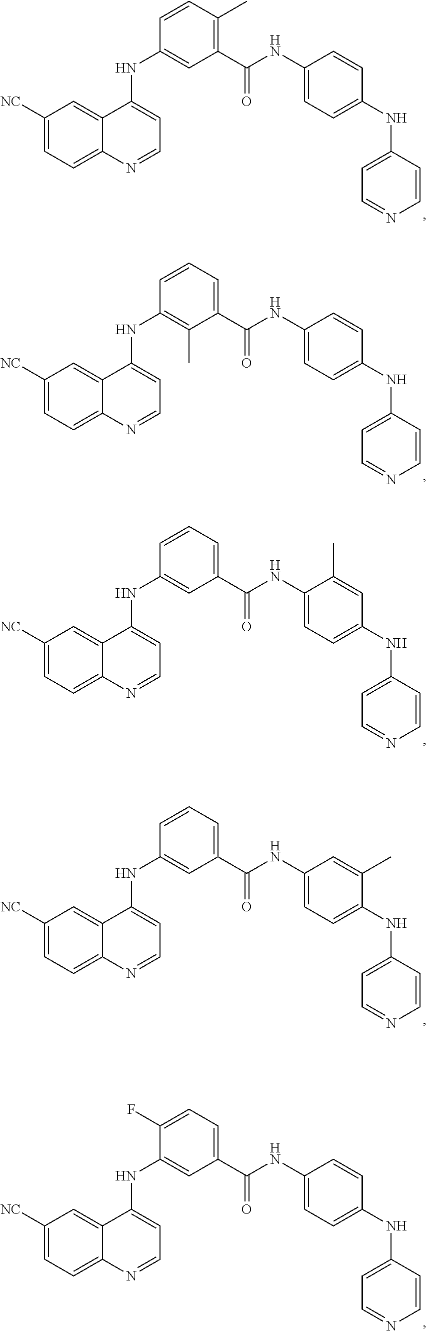 [SMILES: Cc1ccc(Nc2ccnc3ccc(C#N)cc23)cc1C(=O)Nc1ccc(Nc2ccncc2)cc1 ; Cc1c(Nc2ccnc3ccc(C#N)cc23)cccc1C(=O)Nc1ccc(Nc2ccncc2)cc1 ; Cc1cc(Nc2ccncc2)ccc1NC(=O)c1cccc(Nc2ccnc3ccc(C#N)cc23)c1 ; Cc1cc(NC(=O)c2cccc(Nc3ccnc4ccc(C#N)cc34)c2)ccc1Nc1ccncc1 ; N#Cc1ccc2nccc(Nc3cc(C(=O)Nc4ccc(Nc5ccncc5)cc4)ccc3F)c2c1]

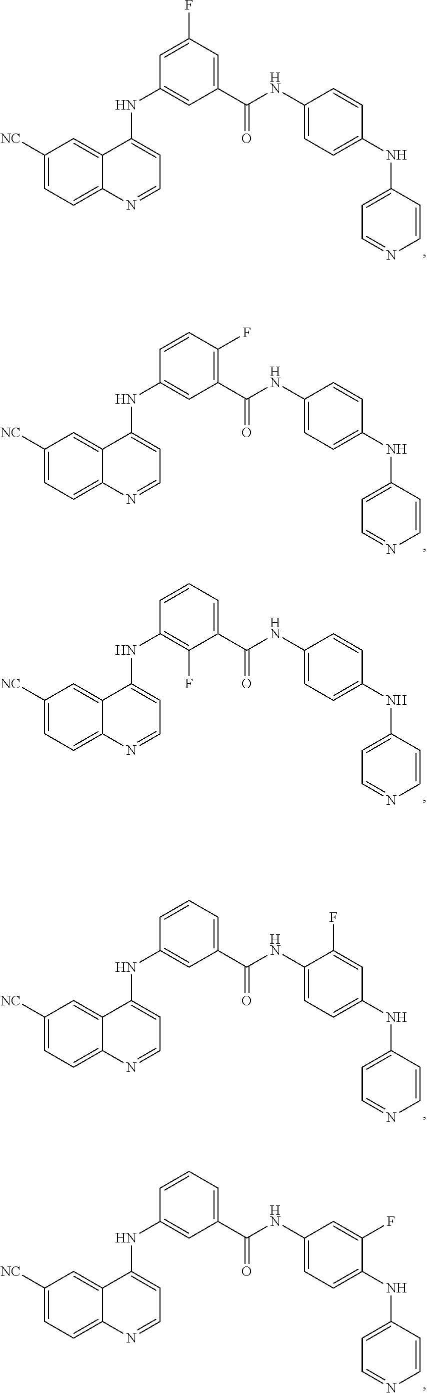 [SMILES: N#Cc1ccc2nccc(Nc3cc(F)cc(C(=O)Nc4ccc(Nc5ccncc5)cc4)c3)c2c1 ; N#Cc1ccc2nccc(Nc3ccc(F)c(C(=O)Nc4ccc(Nc5ccncc5)cc4)c3)c2c1 ; N#Cc1ccc2nccc(Nc3cccc(C(=O)Nc4ccc(Nc5ccncc5)cc4)c3F)c2c1 ; N#Cc1ccc2nccc(Nc3cccc(C(=O)Nc4ccc(Nc5ccncc5)cc4F)c3)c2c1 ; N#Cc1ccc2nccc(Nc3cccc(C(=O)Nc4ccc(Nc5ccncc5)c(F)c4)c3)c2c1]

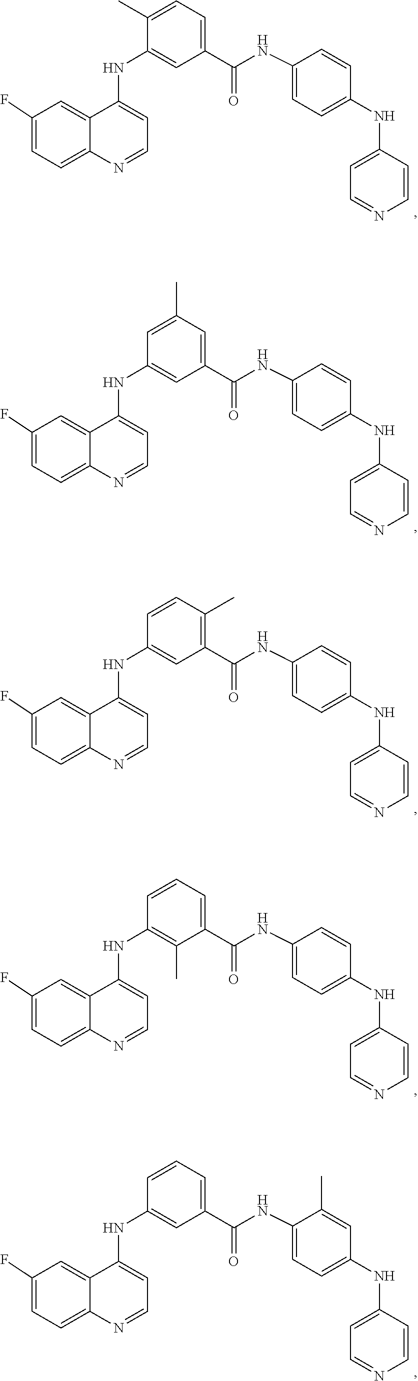 [SMILES: Cc1ccc(C(=O)Nc2ccc(Nc3ccncc3)cc2)cc1Nc1ccnc2ccc(F)cc12 ; Cc1cc(Nc2ccnc3ccc(F)cc23)cc(C(=O)Nc2ccc(Nc3ccncc3)cc2)c1 ; Cc1ccc(Nc2ccnc3ccc(F)cc23)cc1C(=O)Nc1ccc(Nc2ccncc2)cc1 ; Cc1c(Nc2ccnc3ccc(F)cc23)cccc1C(=O)Nc1ccc(Nc2ccncc2)cc1 ; Cc1cc(Nc2ccncc2)ccc1NC(=O)c1cccc(Nc2ccnc3ccc(F)cc23)c1]

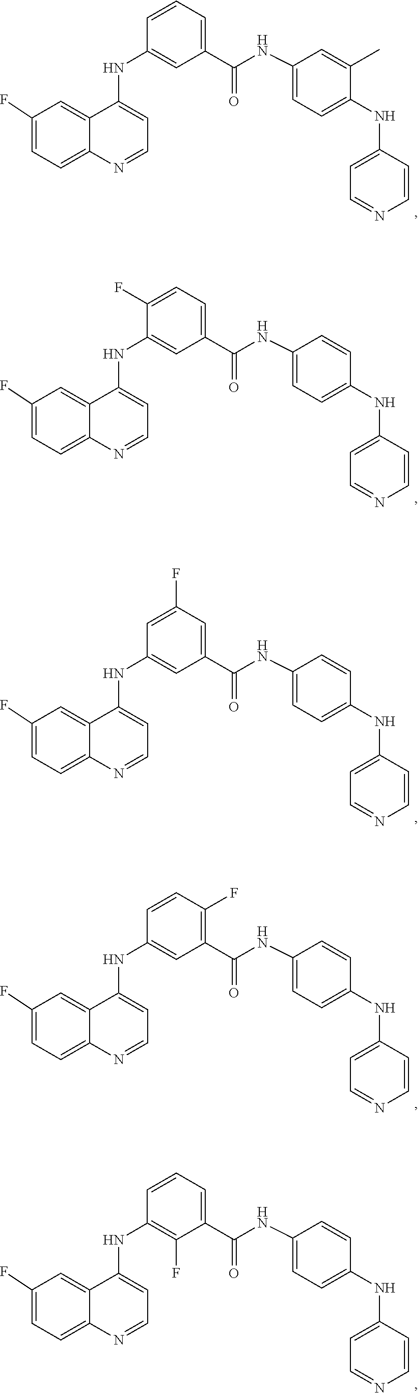 [SMILES: Cc1cc(NC(=O)c2cccc(Nc3ccnc4ccc(F)cc34)c2)ccc1Nc1ccncc1 ; O=C(Nc1ccc(Nc2ccncc2)cc1)c1ccc(F)c(Nc2ccnc3ccc(F)cc23)c1 ; O=C(Nc1ccc(Nc2ccncc2)cc1)c1cc(F)cc(Nc2ccnc3ccc(F)cc23)c1 ; O=C(Nc1ccc(Nc2ccncc2)cc1)c1cc(Nc2ccnc3ccc(F)cc23)ccc1F ; O=C(Nc1ccc(Nc2ccncc2)cc1)c1cccc(Nc2ccnc3ccc(F)cc23)c1F]

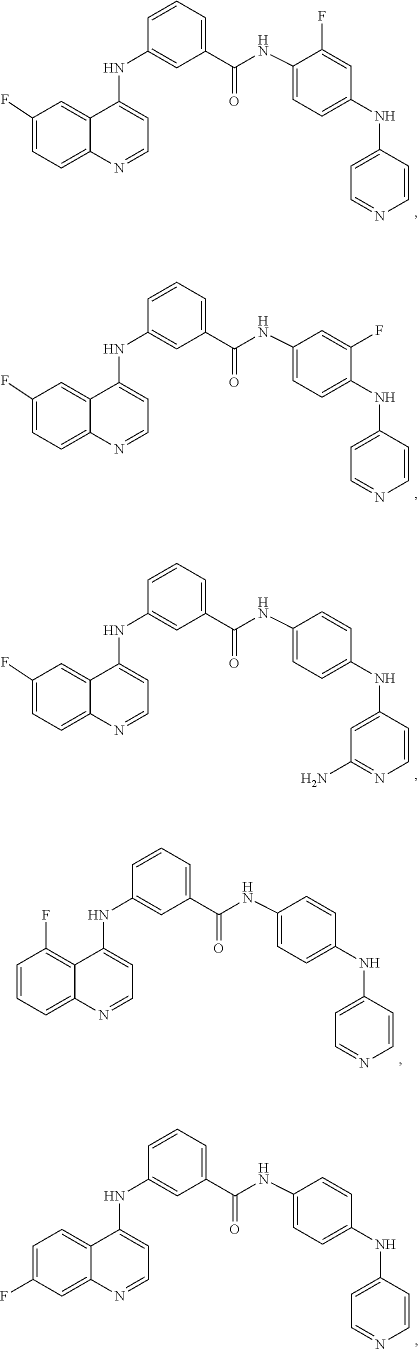 [SMILES: O=C(Nc1ccc(Nc2ccncc2)cc1F)c1cccc(Nc2ccnc3ccc(F)cc23)c1 ; O=C(Nc1ccc(Nc2ccncc2)c(F)c1)c1cccc(Nc2ccnc3ccc(F)cc23)c1 ; Nc1cc(Nc2ccc(NC(=O)c3cccc(Nc4ccnc5ccc(F)cc45)c3)cc2)ccn1 ; O=C(Nc1ccc(Nc2ccncc2)cc1)c1cccc(Nc2ccnc3cccc(F)c23)c1 ; O=C(Nc1ccc(Nc2ccncc2)cc1)c1cccc(Nc2ccnc3cc(F)ccc23)c1]

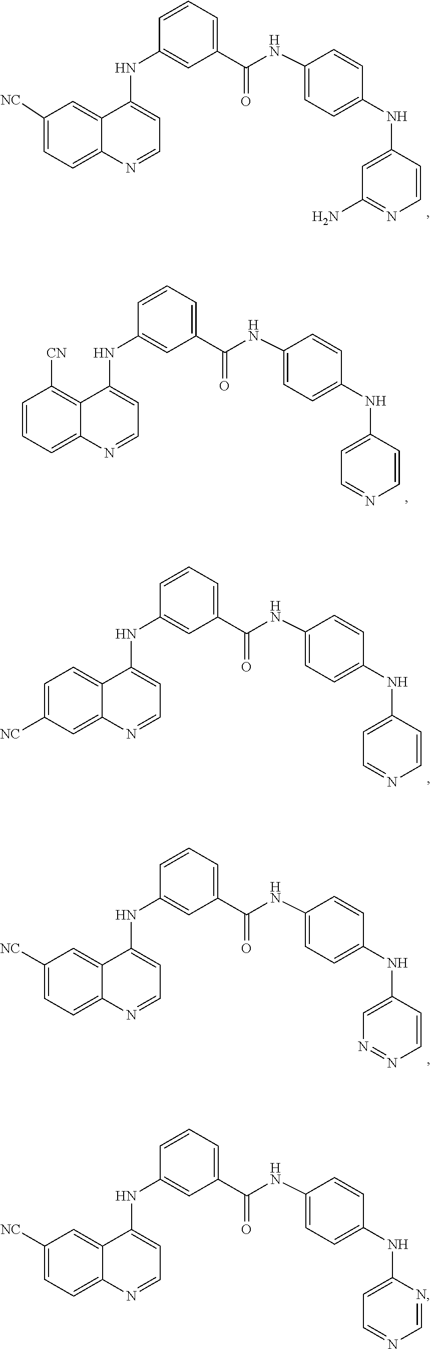 [SMILES: N#Cc1ccc2nccc(Nc3cccc(C(=O)Nc4ccc(Nc5ccnc(N)c5)cc4)c3)c2c1 ; N#Cc1cccc2nccc(Nc3cccc(C(=O)Nc4ccc(Nc5ccncc5)cc4)c3)c12 ; N#Cc1ccc2c(Nc3cccc(C(=O)Nc4ccc(Nc5ccncc5)cc4)c3)ccnc2c1 ; N#Cc1ccc2nccc(Nc3cccc(C(=O)Nc4ccc(Nc5ccnnc5)cc4)c3)c2c1 ; N#Cc1ccc2nccc(Nc3cccc(C(=O)Nc4ccc(Nc5ccncn5)cc4)c3)c2c1]

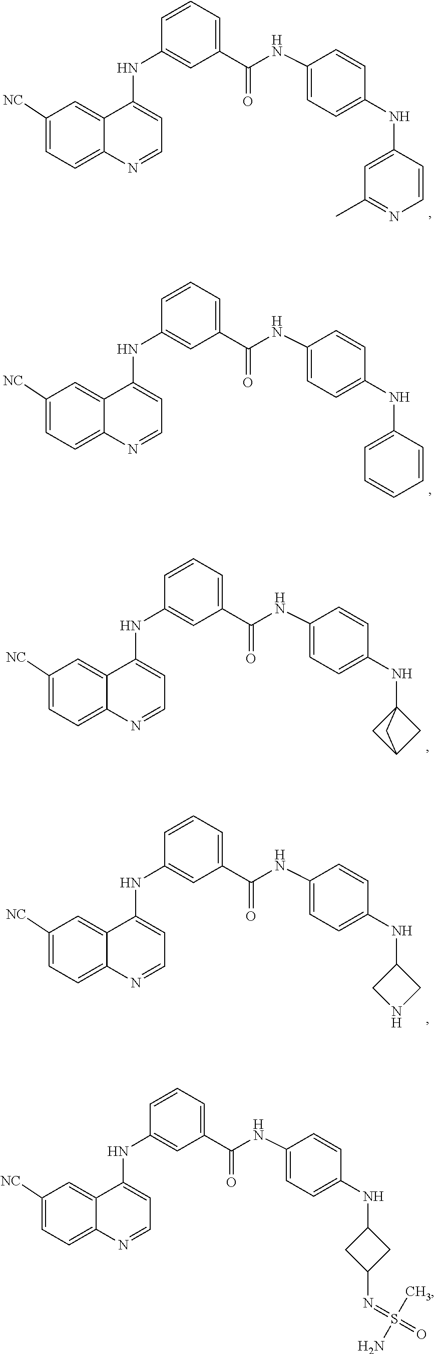 [SMILES: Cc1cc(Nc2ccc(NC(=O)c3cccc(Nc4ccnc5ccc(C#N)cc45)c3)cc2)ccn1 ; N#Cc1ccc2nccc(Nc3cccc(C(=O)Nc4ccc(Nc5ccccc5)cc4)c3)c2c1 ; N#Cc1ccc2nccc(Nc3cccc(C(=O)Nc4ccc(NC56CC(C5)C6)cc4)c3)c2c1 ; N#Cc1ccc2nccc(Nc3cccc(C(=O)Nc4ccc(NC5CNC5)cc4)c3)c2c1 ; CS(N)(=O)=NC1CC(Nc2ccc(NC(=O)c3cccc(Nc4ccnc5ccc(C#N)cc45)c3)cc2)C1]

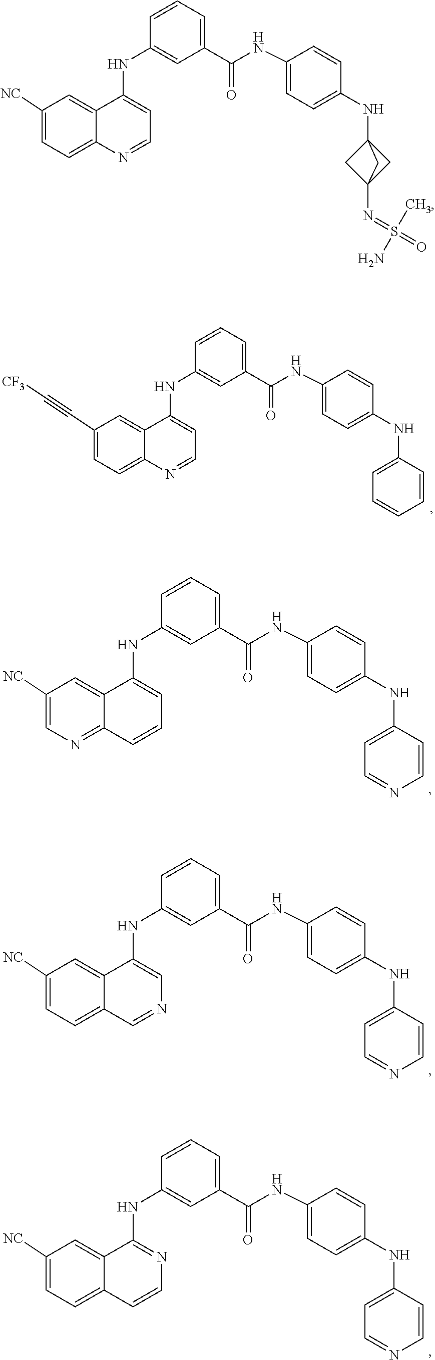 [SMILES: CS(N)(=O)=NC12CC(Nc3ccc(NC(=O)c4cccc(Nc5ccnc6ccc(C#N)cc56)c4)cc3)(C1)C2 ; O=C(Nc1ccc(Nc2ccccc2)cc1)c1cccc(Nc2ccnc3ccc(C#CC(F)(F)F)cc23)c1 ; N#Cc1cnc2cccc(Nc3cccc(C(=O)Nc4ccc(Nc5ccncc5)cc4)c3)c2c1 ; N#Cc1ccc2cncc(Nc3cccc(C(=O)Nc4ccc(Nc5ccncc5)cc4)c3)c2c1 ; N#Cc1ccc2ccnc(Nc3cccc(C(=O)Nc4ccc(Nc5ccncc5)cc4)c3)c2c1]

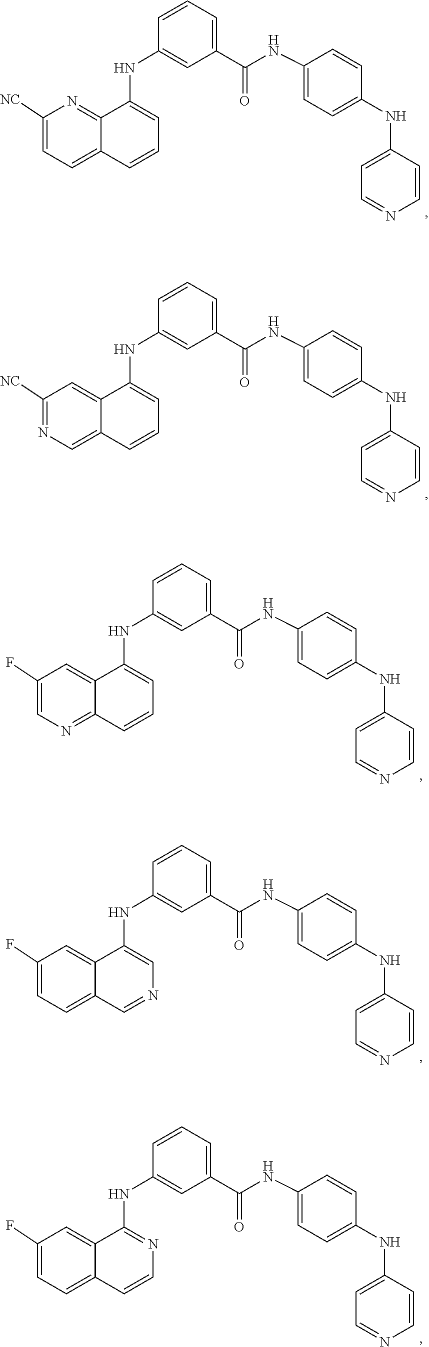 [SMILES: N#Cc1ccc2cccc(Nc3cccc(C(=O)Nc4ccc(Nc5ccncc5)cc4)c3)c2n1 ; N#Cc1cc2c(Nc3cccc(C(=O)Nc4ccc(Nc5ccncc5)cc4)c3)cccc2cn1 ; O=C(Nc1ccc(Nc2ccncc2)cc1)c1cccc(Nc2cccc3ncc(F)cc23)c1 ; O=C(Nc1ccc(Nc2ccncc2)cc1)c1cccc(Nc2cncc3ccc(F)cc23)c1 ; O=C(Nc1ccc(Nc2ccncc2)cc1)c1cccc(Nc2nccc3ccc(F)cc23)c1]

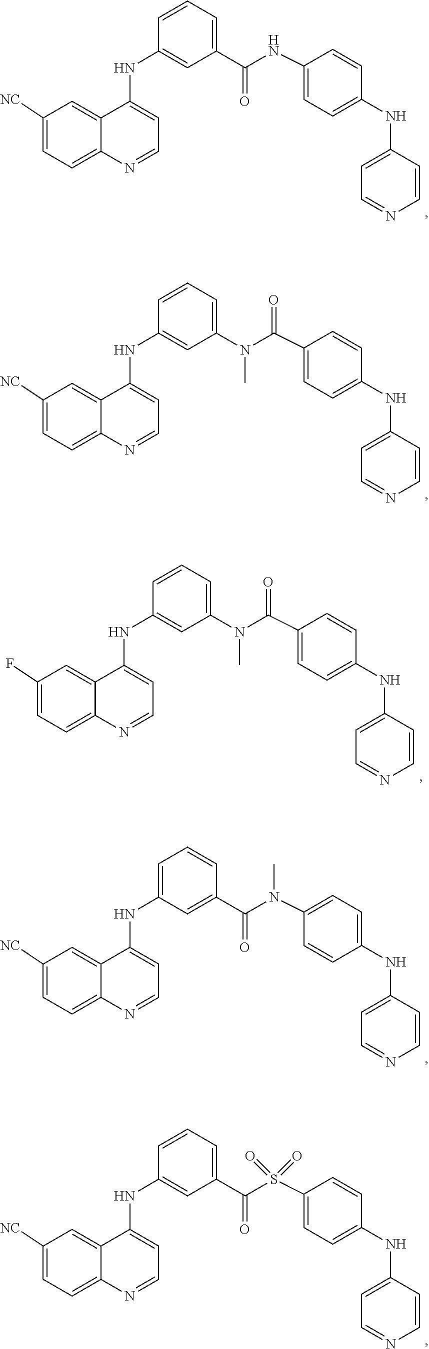 [SMILES: N#Cc1ccc2nccc(Nc3cccc(C(=O)Nc4ccc(Nc5ccncc5)cc4)c3)c2c1 ; CN(C(=O)c1ccc(Nc2ccncc2)cc1)c1cccc(Nc2ccnc3ccc(C#N)cc23)c1 ; CN(C(=O)c1ccc(Nc2ccncc2)cc1)c1cccc(Nc2ccnc3ccc(F)cc23)c1 ; CN(C(=O)c1cccc(Nc2ccnc3ccc(C#N)cc23)c1)c1ccc(Nc2ccncc2)cc1 ; N#Cc1ccc2nccc(Nc3cccc(C(=O)S(=O)(=O)c4ccc(Nc5ccncc5)cc4)c3)c2c1]

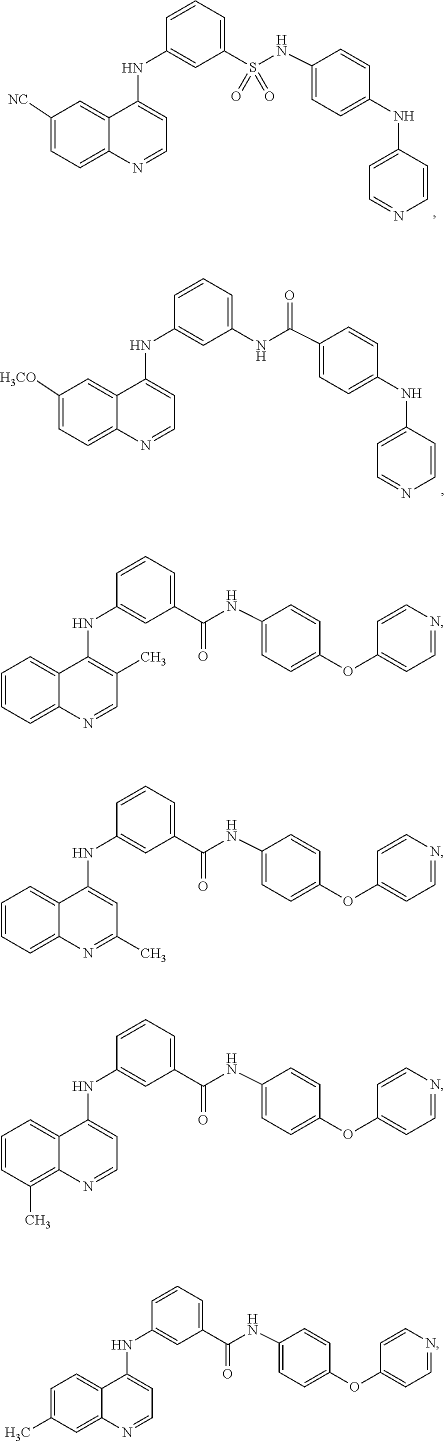 [SMILES: N#Cc1ccc2nccc(Nc3cccc(S(=O)(=O)Nc4ccc(Nc5ccncc5)cc4)c3)c2c1 ; COc1ccc2nccc(Nc3cccc(NC(=O)c4ccc(Nc5ccncc5)cc4)c3)c2c1 ; Cc1cnc2ccccc2c1Nc1cccc(C(=O)Nc2ccc(Oc3ccncc3)cc2)c1 ; Cc1cc(Nc2cccc(C(=O)Nc3ccc(Oc4ccncc4)cc3)c2)c2ccccc2n1 ; Cc1cccc2c(Nc3cccc(C(=O)Nc4ccc(Oc5ccncc5)cc4)c3)ccnc12 ; Cc1ccc2c(Nc3cccc(C(=O)Nc4ccc(Oc5ccncc5)cc4)c3)ccnc2c1]

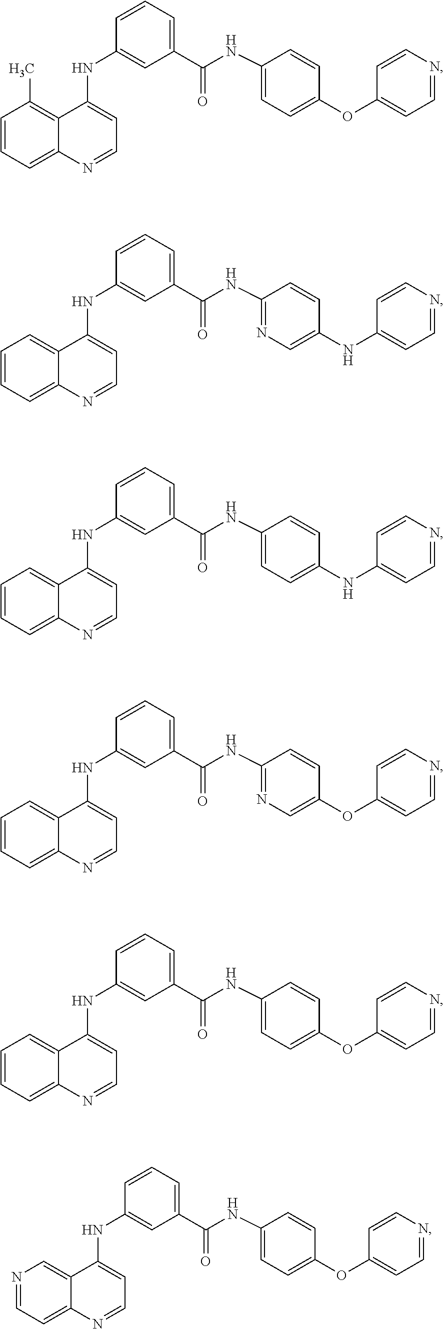 [SMILES: Cc1cccc2nccc(Nc3cccc(C(=O)Nc4ccc(Oc5ccncc5)cc4)c3)c12 ; O=C(Nc1ccc(Nc2ccncc2)cn1)c1cccc(Nc2ccnc3ccccc23)c1 ; O=C(Nc1ccc(Nc2ccncc2)cc1)c1cccc(Nc2ccnc3ccccc23)c1 ; O=C(Nc1ccc(Oc2ccncc2)cn1)c1cccc(Nc2ccnc3ccccc23)c1 ; O=C(Nc1ccc(Oc2ccncc2)cc1)c1cccc(Nc2ccnc3ccccc23)c1 ; O=C(Nc1ccc(Oc2ccncc2)cc1)c1cccc(Nc2ccnc3ccncc23)c1]

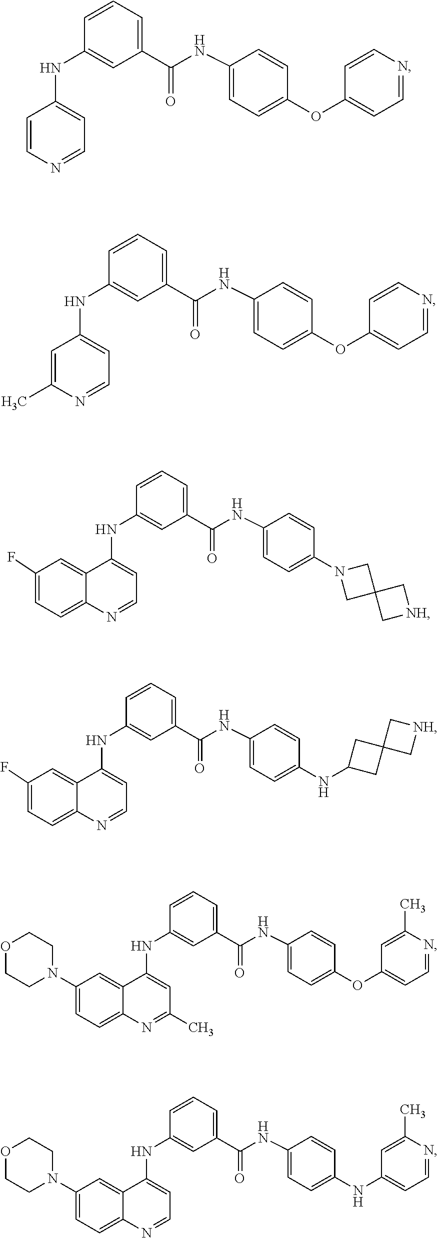 [SMILES: O=C(Nc1ccc(Oc2ccncc2)cc1)c1cccc(Nc2ccncc2)c1 ; Cc1cc(Nc2cccc(C(=O)Nc3ccc(Oc4ccncc4)cc3)c2)ccn1 ; O=C(Nc1ccc(N2CC3(CNC3)C2)cc1)c1cccc(Nc2ccnc3ccc(F)cc23)c1 ; O=C(Nc1ccc(NC2CC3(CNC3)C2)cc1)c1cccc(Nc2ccnc3ccc(F)cc23)c1 ; Cc1cc(Oc2ccc(NC(=O)c3cccc(Nc4cc(C)nc5ccc(N6CCOCC6)cc45)c3)cc2)ccn1 ; Cc1cc(Nc2ccc(NC(=O)c3cccc(Nc4ccnc5ccc(N6CCOCC6)cc45)c3)cc2)ccn1]

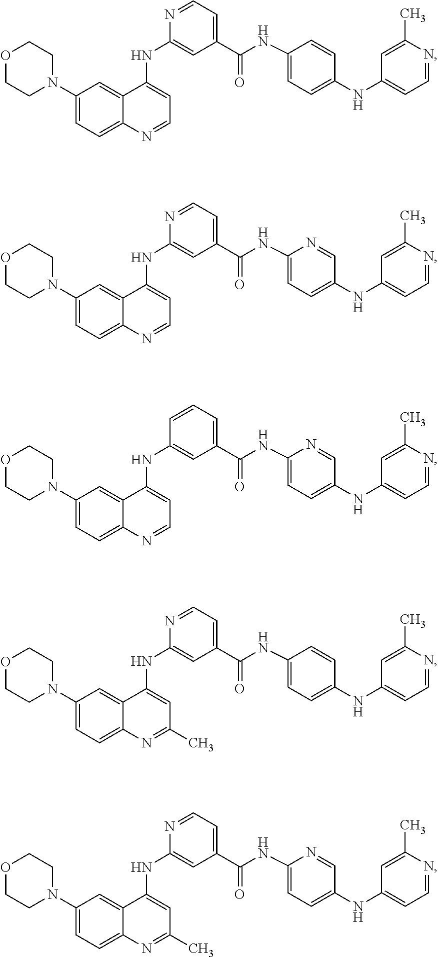 [SMILES: Cc1cc(Nc2ccc(NC(=O)c3ccnc(Nc4ccnc5ccc(N6CCOCC6)cc45)c3)cc2)ccn1 ; Cc1cc(Nc2ccc(NC(=O)c3ccnc(Nc4ccnc5ccc(N6CCOCC6)cc45)c3)nc2)ccn1 ; Cc1cc(Nc2ccc(NC(=O)c3cccc(Nc4ccnc5ccc(N6CCOCC6)cc45)c3)nc2)ccn1 ; Cc1cc(Nc2ccc(NC(=O)c3ccnc(Nc4cc(C)nc5ccc(N6CCOCC6)cc45)c3)cc2)ccn1 ; Cc1cc(Nc2ccc(NC(=O)c3ccnc(Nc4cc(C)nc5ccc(N6CCOCC6)cc45)c3)nc2)ccn1]

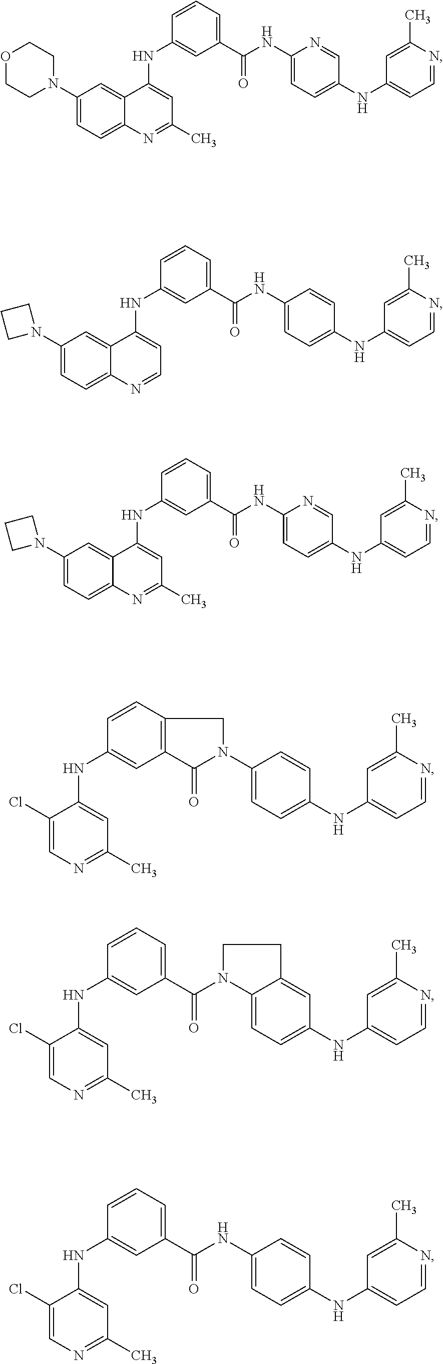 [SMILES: Cc1cc(Nc2ccc(NC(=O)c3cccc(Nc4cc(C)nc5ccc(N6CCOCC6)cc45)c3)nc2)ccn1 ; Cc1cc(Nc2ccc(NC(=O)c3cccc(Nc4ccnc5ccc(N6CCC6)cc45)c3)cc2)ccn1 ; Cc1cc(Nc2ccc(NC(=O)c3cccc(Nc4cc(C)nc5ccc(N6CCC6)cc45)c3)nc2)ccn1 ; Cc1cc(Nc2ccc(N3Cc4ccc(Nc5cc(C)ncc5Cl)cc4C3=O)cc2)ccn1 ; Cc1cc(Nc2ccc3c(c2)CCN3C(=O)c2cccc(Nc3cc(C)ncc3Cl)c2)ccn1 ; Cc1cc(Nc2ccc(NC(=O)c3cccc(Nc4cc(C)ncc4Cl)c3)cc2)ccn1]

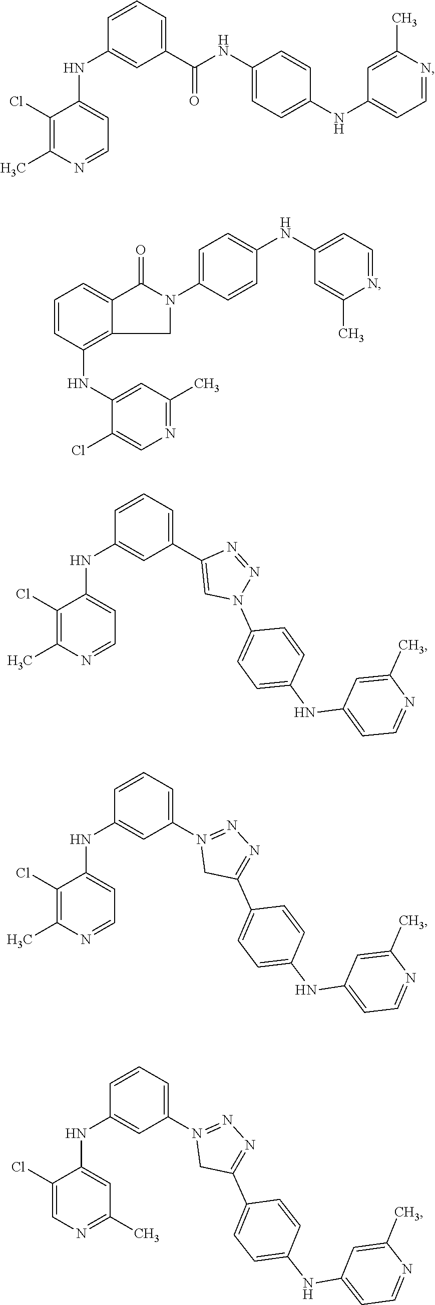

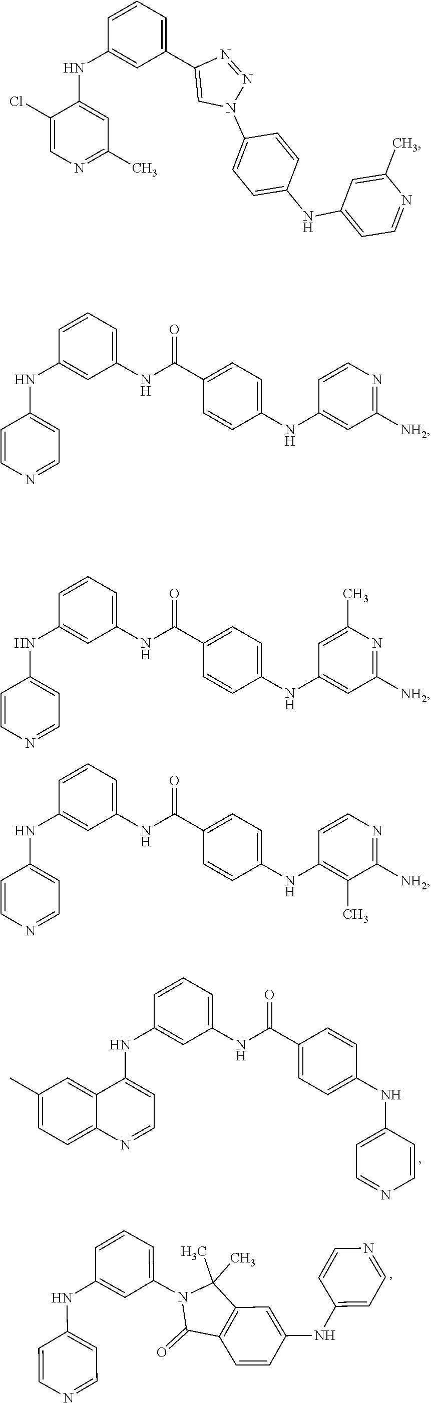 [SMILES: Cc1cc(Nc2ccc(-n3cc(-c4cccc(Nc5cc(C)ncc5Cl)c4)nn3)cc2)ccn1 ; Nc1cc(Nc2ccc(C(=O)Nc3cccc(Nc4ccncc4)c3)cc2)ccn1 ; Cc1cc(Nc2ccc(C(=O)Nc3cccc(Nc4ccncc4)c3)cc2)cc(N)n1 ; Cc1c(Nc2ccc(C(=O)Nc3cccc(Nc4ccncc4)c3)cc2)ccnc1N ; Cc1ccc2nccc(Nc3cccc(NC(=O)c4ccc(Nc5ccncc5)cc4)c3)c2c1 ; CC1(C)c2cc(Nc3ccncc3)ccc2C(=O)N1c1cccc(Nc2ccncc2)c1]

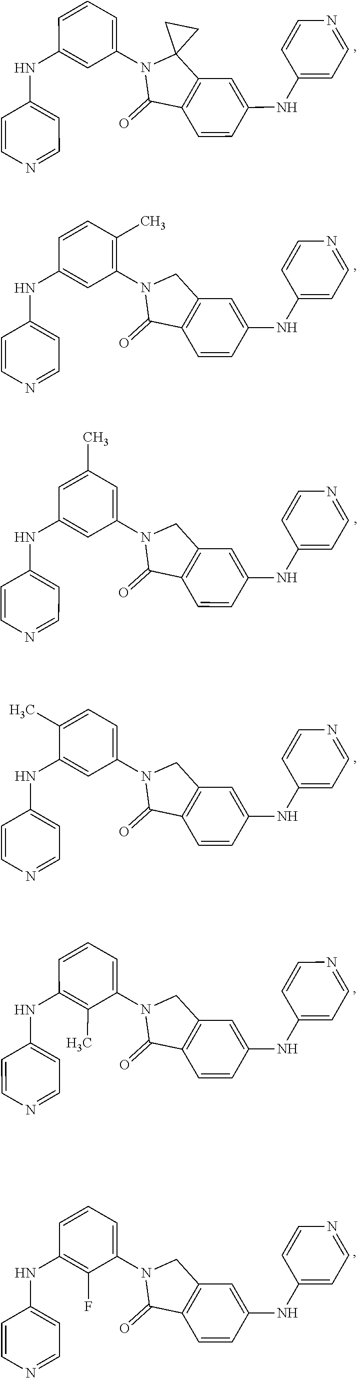 [SMILES: O=C1c2ccc(Nc3ccncc3)cc2C2(CC2)N1c1cccc(Nc2ccncc2)c1 ; Cc1ccc(Nc2ccncc2)cc1N1Cc2cc(Nc3ccncc3)ccc2C1=O ; Cc1cc(Nc2ccncc2)cc(N2Cc3cc(Nc4ccncc4)ccc3C2=O)c1 ; Cc1ccc(N2Cc3cc(Nc4ccncc4)ccc3C2=O)cc1Nc1ccncc1 ; Cc1c(Nc2ccncc2)cccc1N1Cc2cc(Nc3ccncc3)ccc2C1=O ; O=C1c2ccc(Nc3ccncc3)cc2CN1c1cccc(Nc2ccncc2)c1F]

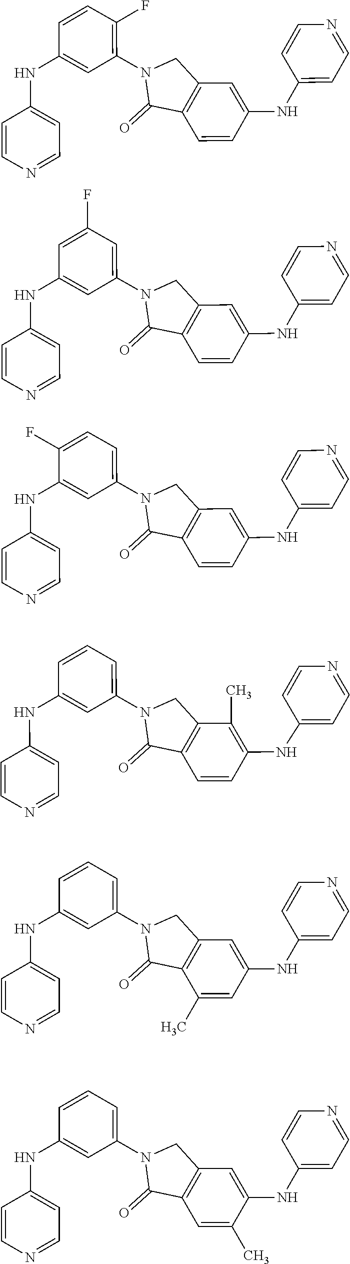

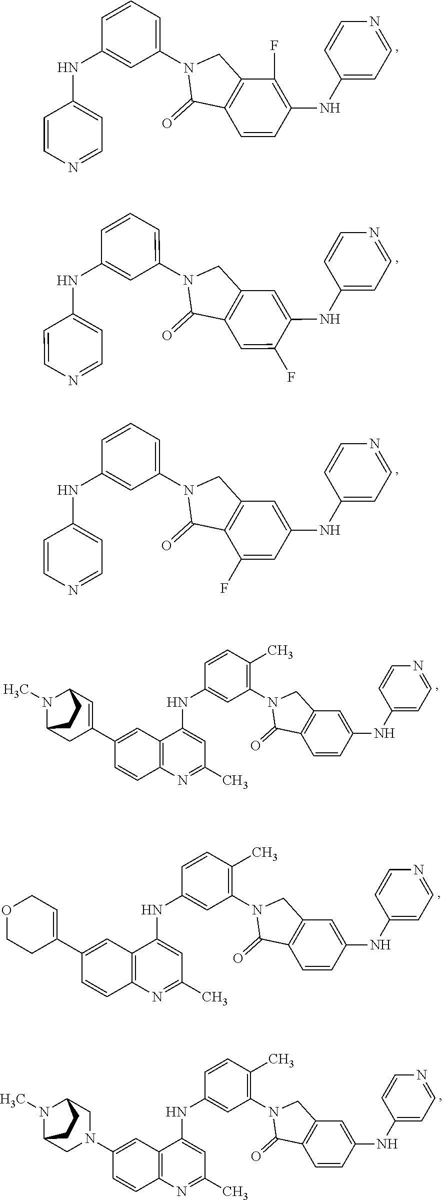

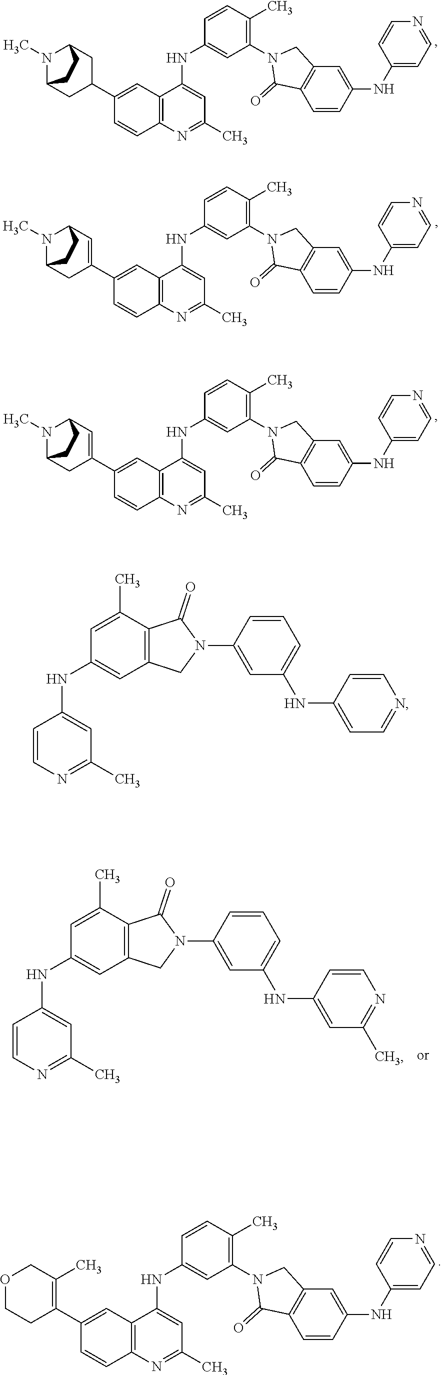

In some embodiments, the compound of Formula Ia is

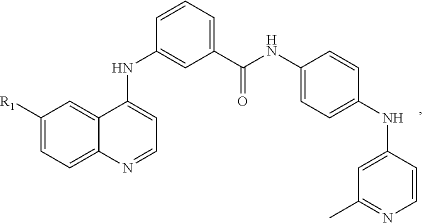

where R₁ is —CONH₂, —SO₂NH₂, —SO₂CH₃, or

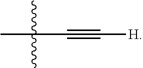

In some embodiments, the compound of Formula Ia is

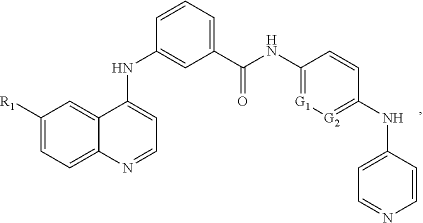

where R₁ is —CN or —F, and G₁ and G₂ are either —N— and —CH—, or —CH— and —N—.

In some embodiments, the compound of Formula Ia is

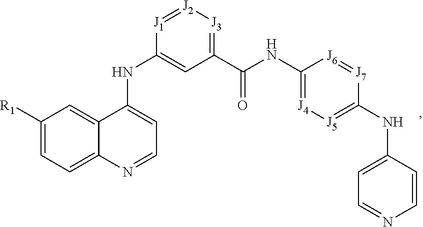

where R₁ is —CONH₂, —SO₂NH₂, —SO₂CH₃, or

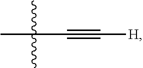

and each of J₁, J₂, J₃, J₄, J₅, J₆, and J₇ is independently —N— or —CF.

In some embodiments, the compound of Formula Ia is

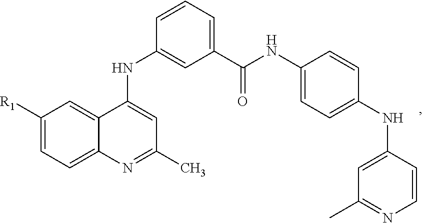

where R₁ is —CONH₂, —SO₂NH₂, —SO₂CH₃,

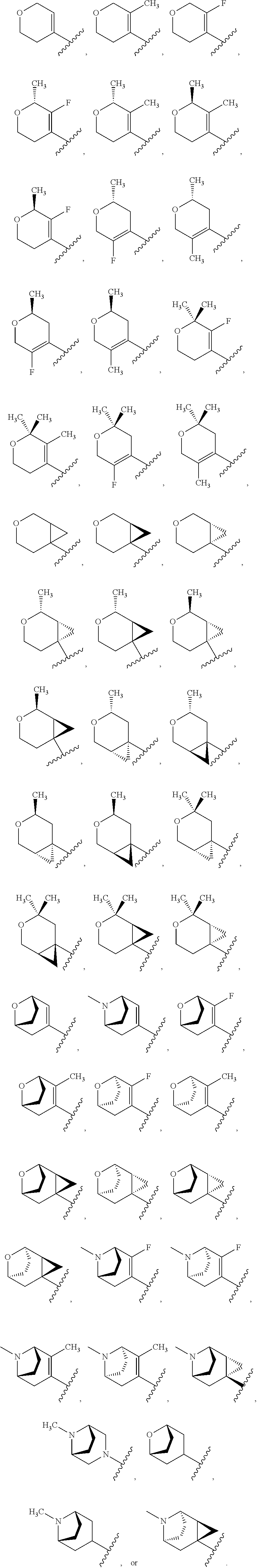

In some embodiments, the compound of Formula Ia is

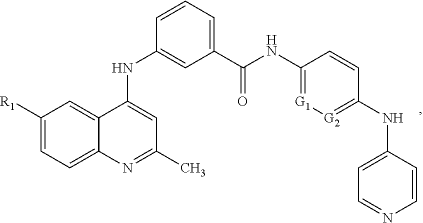

where R₁ is

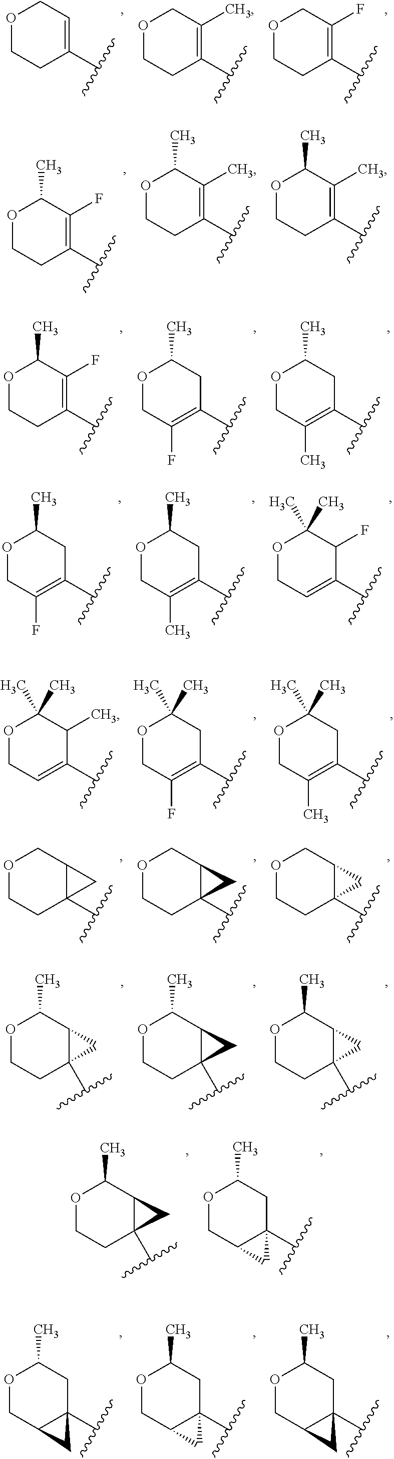

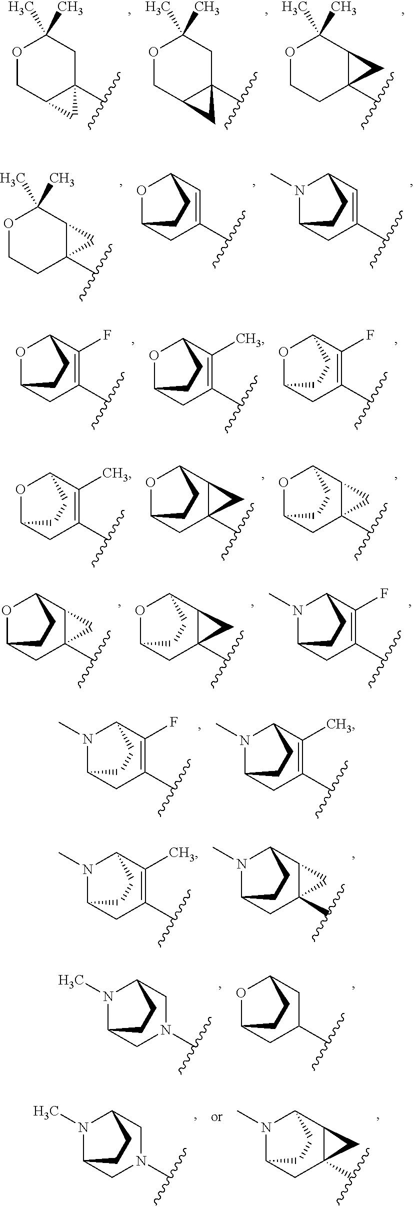

and G₁ and G₂ are either —N— and —CH—, or —CH— and —N—.

In some embodiments, the compound of Formula Ia is

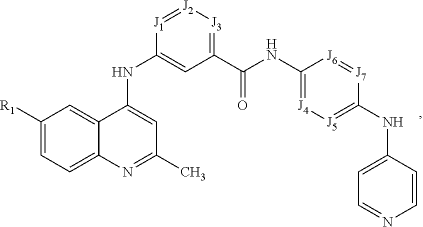

where R₁ is

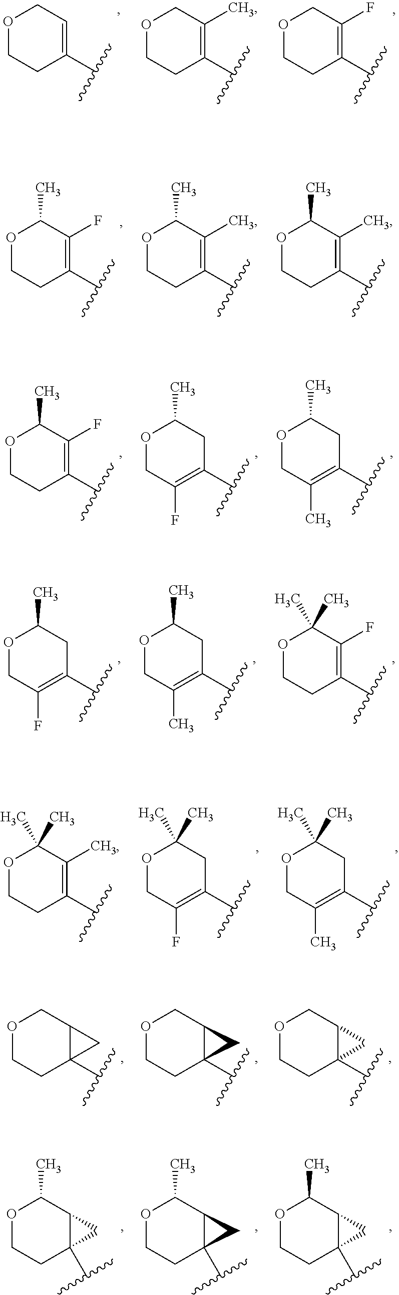

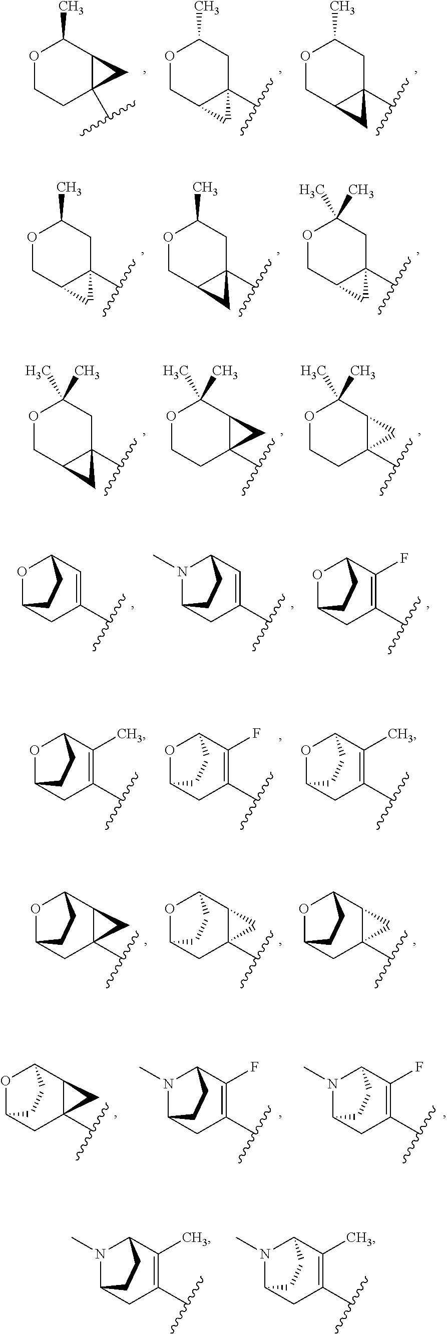

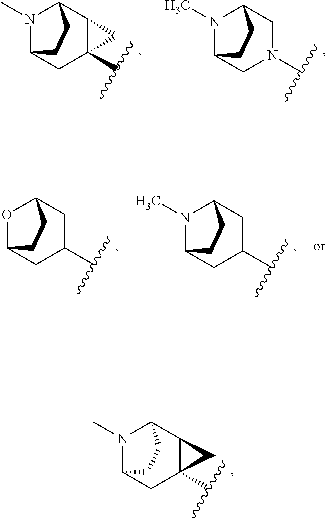

and each of J₁, J₂, J₃, J₄, J₅, J₆, and J₇ is independently —N— or —CF.

In some embodiments, the compound of Formula Ib is

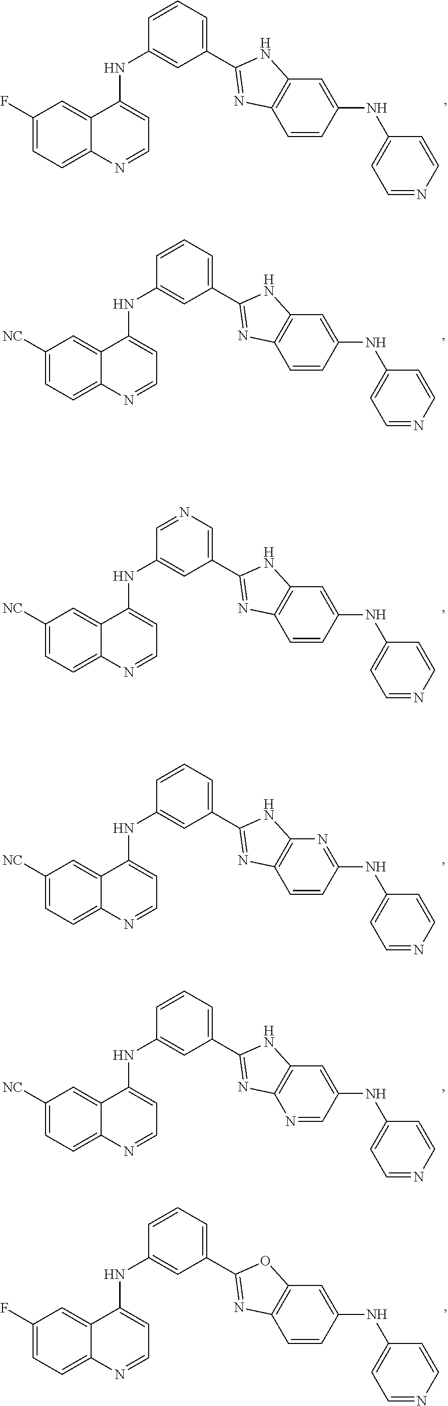 [SMILES: Fc1ccc2nccc(Nc3cccc(-c4nc5ccc(Nc6ccncc6)cc5[nH]4)c3)c2c1 ; N#Cc1ccc2nccc(Nc3cccc(-c4nc5ccc(Nc6ccncc6)cc5[nH]4)c3)c2c1 ; N#Cc1ccc2nccc(Nc3cncc(-c4nc5ccc(Nc6ccncc6)cc5[nH]4)c3)c2c1 ; N#Cc1ccc2nccc(Nc3cccc(-c4nc5ccc(Nc6ccncc6)nc5[nH]4)c3)c2c1 ; N#Cc1ccc2nccc(Nc3cccc(-c4nc5ncc(Nc6ccncc6)cc5[nH]4)c3)c2c1 ; Fc1ccc2nccc(Nc3cccc(-c4nc5ccc(Nc6ccncc6)cc5o4)c3)c2c1]

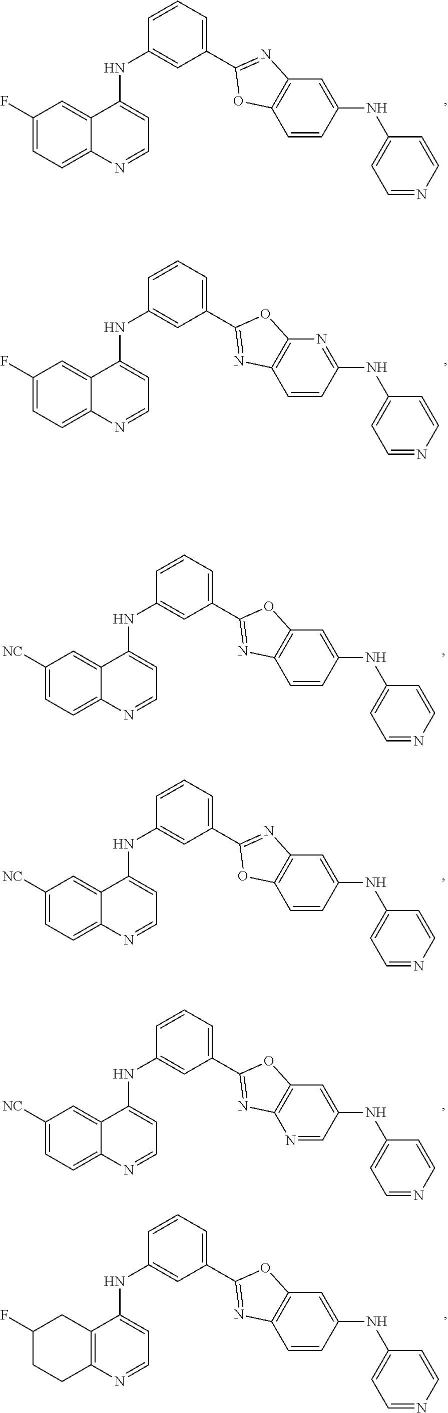 [SMILES: Fc1ccc2nccc(Nc3cccc(-c4nc5cc(Nc6ccncc6)ccc5o4)c3)c2c1 ; Fc1ccc2nccc(Nc3cccc(-c4nc5ccc(Nc6ccncc6)nc5o4)c3)c2c1 ; N#Cc1ccc2nccc(Nc3cccc(-c4nc5ccc(Nc6ccncc6)cc5o4)c3)c2c1 ; N#Cc1ccc2nccc(Nc3cccc(-c4nc5cc(Nc6ccncc6)ccc5o4)c3)c2c1 ; N#Cc1ccc2nccc(Nc3cccc(-c4nc5ncc(Nc6ccncc6)cc5o4)c3)c2c1 ; FC1CCc2nccc(Nc3cccc(-c4nc5ccc(Nc6ccncc6)cc5o4)c3)c2C1]

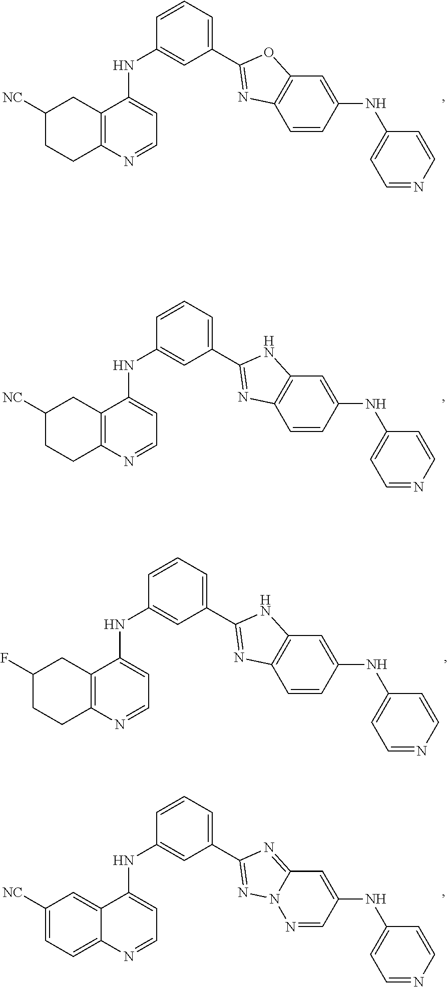 [SMILES: N#CC1CCc2nccc(Nc3cccc(-c4nc5ccc(Nc6ccncc6)cc5o4)c3)c2C1 ; N#CC1CCc2nccc(Nc3cccc(-c4nc5ccc(Nc6ccncc6)cc5[nH]4)c3)c2C1 ; FC1CCc2nccc(Nc3cccc(-c4nc5ccc(Nc6ccncc6)cc5[nH]4)c3)c2C1 ; N#Cc1ccc2nccc(Nc3cccc(-c4nc5cc(Nc6ccncc6)cnn5n4)c3)c2c1]

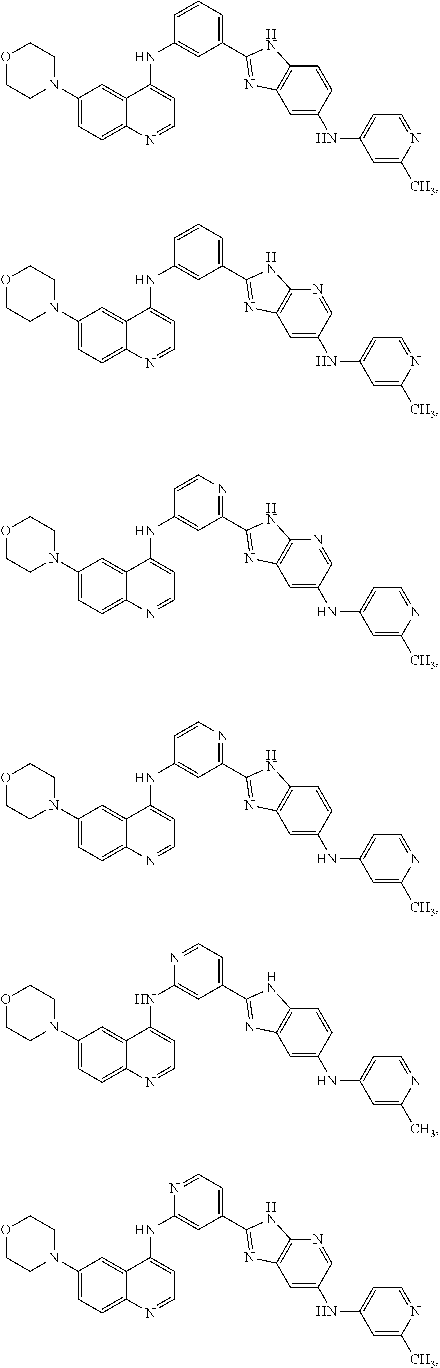 [SMILES: Cc1cc(Nc2ccc3[nH]c(-c4cccc(Nc5ccnc6ccc(N7CCOCC7)cc56)c4)nc3c2)ccn1 ; Cc1cc(Nc2cnc3[nH]c(-c4cccc(Nc5ccnc6ccc(N7CCOCC7)cc56)c4)nc3c2)ccn1 ; Cc1cc(Nc2cnc3[nH]c(-c4cc(Nc5ccnc6ccc(N7CCOCC7)cc56)ccn4)nc3c2)ccn1 ; Cc1cc(Nc2ccc3[nH]c(-c4cc(Nc5ccnc6ccc(N7CCOCC7)cc56)ccn4)nc3c2)ccn1 ; Cc1cc(Nc2ccc3[nH]c(-c4ccnc(Nc5ccnc6ccc(N7CCOCC7)cc56)c4)nc3c2)ccn1 ; Cc1cc(Nc2cnc3[nH]c(-c4ccnc(Nc5ccnc6ccc(N7CCOCC7)cc56)c4)nc3c2)ccn1]

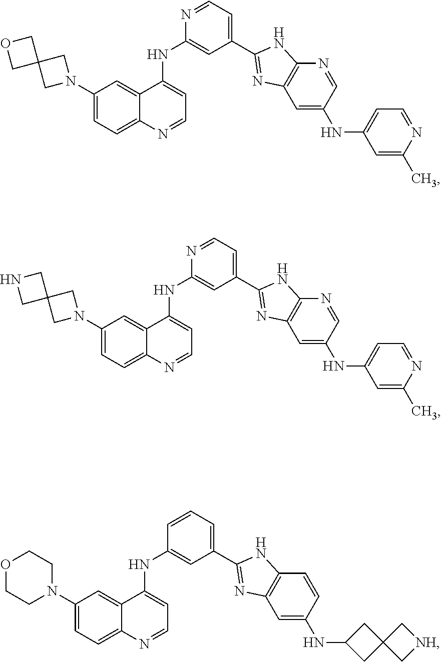 [SMILES: Cc1cc(Nc2cnc3[nH]c(-c4ccnc(Nc5ccnc6ccc(N7CC8(COC8)C7)cc56)c4)nc3c2)ccn1 ; Cc1cc(Nc2cnc3[nH]c(-c4ccnc(Nc5ccnc6ccc(N7CC8(CNC8)C7)cc56)c4)nc3c2)ccn1 ; c1cc(Nc2ccnc3ccc(N4CCOCC4)cc23)cc(-c2nc3cc(NC4CC5(CNC5)C4)ccc3[nH]2)c1]

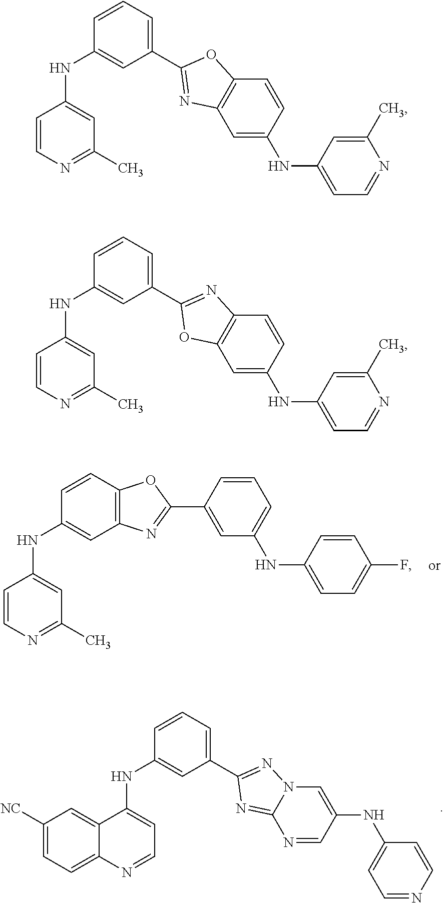 [SMILES: Cc1cc(Nc2cccc(-c3nc4cc(Nc5ccnc(C)c5)ccc4o3)c2)ccn1 ; Cc1cc(Nc2cccc(-c3nc4ccc(Nc5ccnc(C)c5)cc4o3)c2)ccn1 ; Cc1cc(Nc2ccc3oc(-c4cccc(Nc5ccc(F)cc5)c4)nc3c2)ccn1 ; N#Cc1ccc2nccc(Nc3cccc(-c4nc5ncc(Nc6ccncc6)cn5n4)c3)c2c1]

In some embodiments, the compound of Formula Ic is

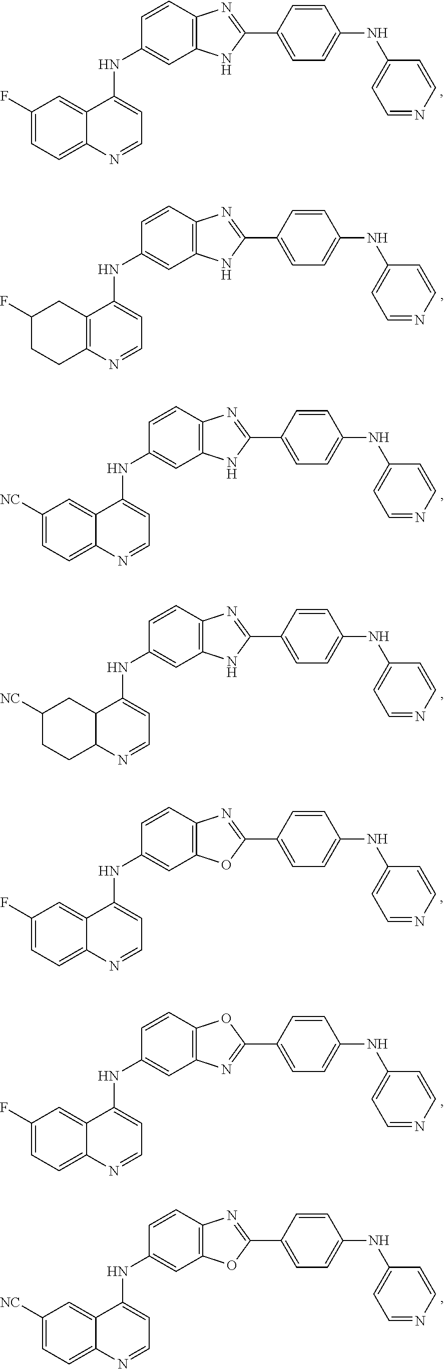

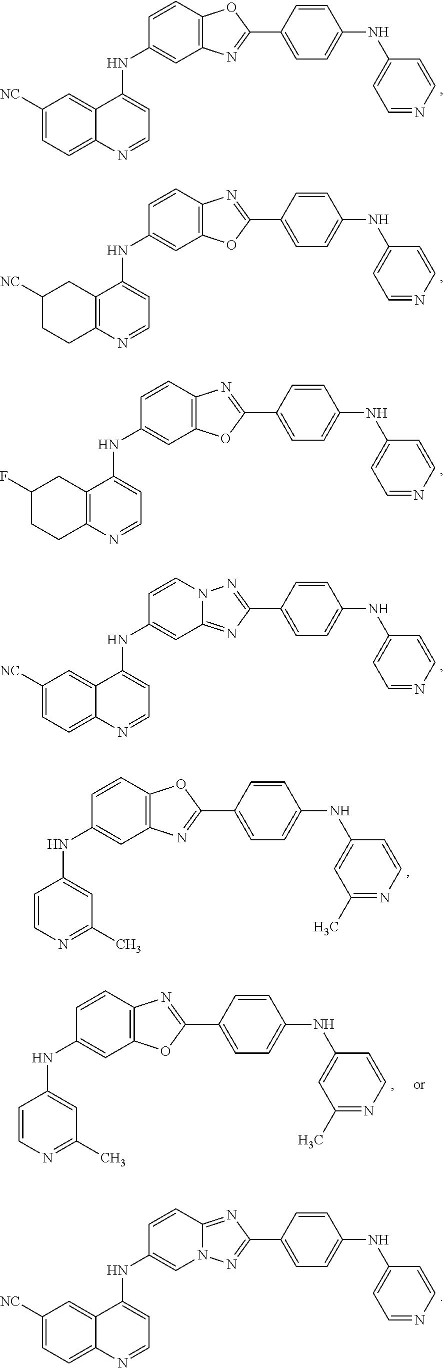

In some embodiments, the compound is

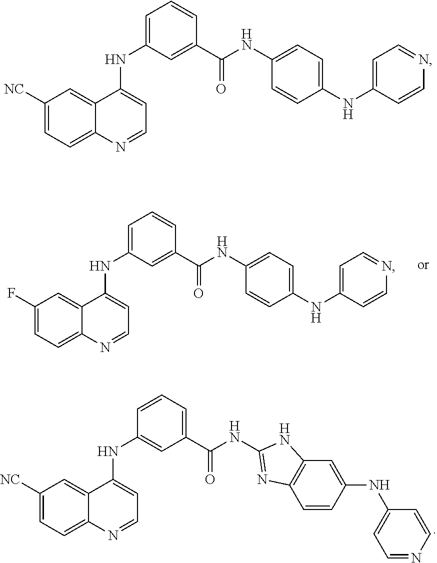 [SMILES: N#Cc1ccc2nccc(Nc3cccc(C(=O)Nc4ccc(Nc5ccncc5)cc4)c3)c2c1 ; O=C(Nc1ccc(Nc2ccncc2)cc1)c1cccc(Nc2ccnc3ccc(F)cc23)c1 ; "or" ; N#Cc1ccc2nccc(Nc3cccc(C(=O)Nc4nc5ccc(Nc6ccncc6)cc5[nH]4)c3)c2c1]

In some embodiments, the compound is

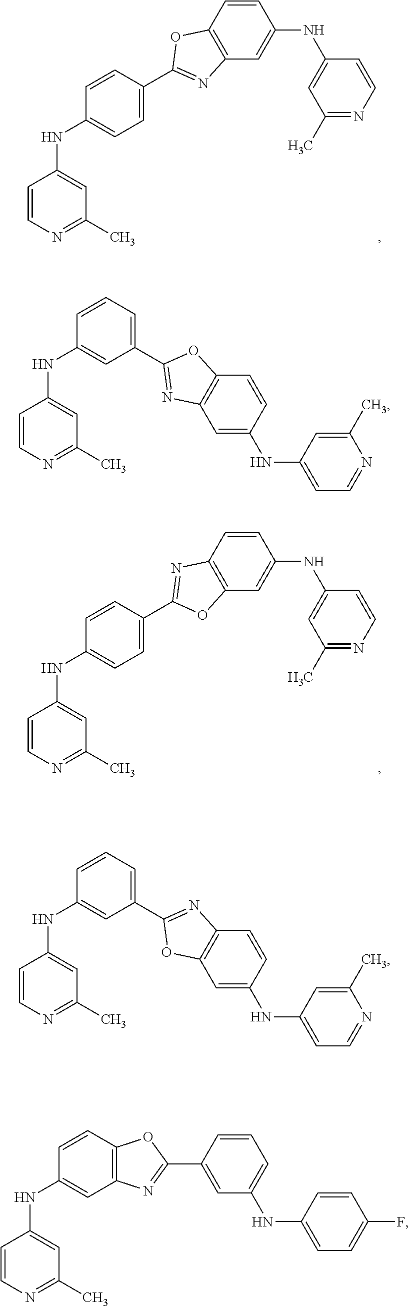 [SMILES: Cc1cc(Nc2ccc(-c3nc4cc(Nc5ccnc(C)c5)ccc4o3)cc2)ccn1 ; Cc1cc(Nc2cccc(-c3nc4cc(Nc5ccnc(C)c5)ccc4o3)c2)ccn1 ; Cc1cc(Nc2ccc(-c3nc4ccc(Nc5ccnc(C)c5)cc4o3)cc2)ccn1 ; Cc1cc(Nc2cccc(-c3nc4ccc(Nc5ccnc(C)c5)cc4o3)c2)ccn1 ; Cc1cc(Nc2ccc3oc(-c4cccc(Nc5ccc(F)cc5)c4)nc3c2)ccn1]

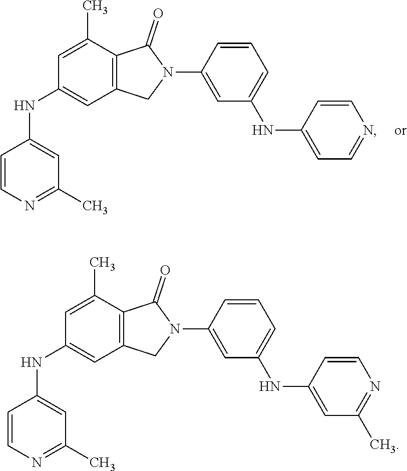 [SMILES: Cc1cc(Nc2cc(C)c3c(c2)CN(c2cccc(Nc4ccncc4)c2)C3=O)ccn1 ; Cc1cc(Nc2cccc(N3Cc4cc(Nc5ccnc(C)c5)cc(C)c4C3=O)c2)ccn1]

In any one of the embodiments disclosed herein, the compound is selected from the group consisting of Compounds 2-45 in Examples 2-45, respectively.

In any one of the embodiments disclosed herein, the compound is selected from the group consisting of Compounds 22, 31, and 39-45 in Examples 22, 31, and 39-45, respectively.

In any one of the embodiments disclosed herein, the compound is selected from the group consisting of Compounds 3, 18-21, 26, 27, 30, 32-34, and 37-45 as shown in Table 1.

In any one of the embodiments disclosed herein, the compound is Compound 2 as shown in Table 2.

### Prodrugs

In some embodiments, any one of the compounds described herein may be made into a prodrug by attaching to one or more functional groups therein a cleavable moiety. See, e.g., J. Med. Chem., Vol. 61, pp. 62-80 (2018); J. Med. Chem., Vol. 61, pp. 6308-6327 (2018); and J. Med. Chem., Vol. 61, pp. 3918-3929 (2018). In some embodiments, the moiety is cleavable upon exposure to a stimulus. Non-limiting examples of such a stimulus include temperature, electromagnetic radiation, sonic vibrations, pH, solvents, and substances and processes found on or in living organisms. In some embodiments, the cleavable moiety is removed upon contact with a living organism. In some embodiments, the cleavable moiety is removed upon contact with an enzyme. In some, embodiments, the cleavable moiety is removed upon contact with alkaline phosphatase. In some embodiments, the cleavable moiety is a phosphonooxymethyl moiety that is cleaved as illustrated in Scheme A below.

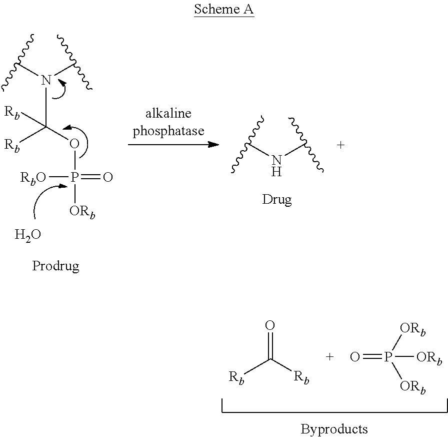

### Methods of Modulating Akt3

Akt3, also referred to as RAC-gamma serine/threonine-protein kinase, is an enzyme that, in humans, is encoded by the Akt3 gene. Akt kinases are known to be regulators of cell signaling in response to insulin and growth factors and are associated with a broad range of biological processes, including, but not limited to, cell proliferation, differentiation, apoptosis, and tumorigenesis, as well as glycogen synthesis and glucose uptake. Akt3 has been shown to be stimulated by platelet-derived growth factor (“PDGF”), insulin, and insulin-like growth factor 1 (“IGF1”).

Akt3 kinase activity mediates serine and/or threonine phosphorylation of a range of downstream substrates. Nucleic acid sequences for Akt3 are known in the art. See, for example, Genbank accession no. AF124141.1: Homo sapiens protein kinase B gamma mRNA, complete cds, which is specifically incorporated by reference in its entirety, and provides the following nucleic acid sequence:

(SEQ ID NO: 1)
AGGGGAGTCATCATGAGCGATGTTACCATTGTGAAGGAAGGTTGGGTTCAGAAGAGGGGA
GAATATATAAAAAACTGGAGGCCAAGATACTTCCTTTTGAAGACAGATGGCTCATTCATA
GGATATAAAGAGAAACCTCAAGATGTGGATTTACCTTATCCCCTCAACAACTTTTCAGTG
GCAAAATGCCAGTTAATGAAAACAGAACGACCAAAGCCAAACACATTTATAATCAGATGT
CTCCAGTGGACTACTGTTATAGAGAGAACATTTCATGTAGATACTCCAGAGGAAAGGGAA
GAATGGACAGAAGCTATCCAGGCTGTAGCAGACAGACTGCAGAGGCAAGAAGAGGAGAGA
ATGAATTGTAGTCCAACTTCACAAATTGATAATATAGGAGAGGAAGAGATGGATGCCTCT
ACAACCCATCATAAAAGAAAGACAATGAATGATTTTGACTATTTGAAACTACTAGGTAAA
GGCACTTTTGGGAAAGTTATTTTGGTTCGAGAGAAGGCAAGTGGAAAATACTATGCTATG
AAGATTCTGAAGAAAGAAGTCATTATTGCAAAGGATGAAGTGGCACACACTCTAACTGAA
AGCAGAGTATTAAAGAACACTAGACATCCCTTTTTAACATCCTTGAAATATTCCTTCCAG
ACAAAAGACCGTTTGTGTTTTGTGATGGAATATGTTAATGGGGGCGAGCTGTTTTTCCAT
TTGTCGAGAGAGCGGGTGTTCTCTGAGGACCGCACACGTTTCTATGGTGCAGAAATTGTC
TCTGCCTTGGACTATCTACATTCCGGAAAGATTGTGTACCGTGATCTCAAGTTGGAGAAT
CTAATGCTGGACAAAGATGGCCACATAAAAATTACAGATTTTGGACTTTGCAAAGAAGGG
ATCACAGATGCAGCCACCATGAAGACATTCTGTGGCACTCCAGAATATCTGGCACCAGAG
GTGTTAGAAGATAATGACTATGGCCGAGCAGTAGACTGGTGGGGCCTAGGGGTTGTCATG
TATGAAATGATGTGTGGGAGGTTACCTTTCTACAACCAGGACCATGAGAAACTTTTTGAA
TTAATATTAATGGAAGACATTAAATTTCCTCGAACACTCTCTTCAGATGCAAAATCATTG
CTTTCAGGGCTCTTGATAAAGGATCCAAATAAACGCCTTGGTGGAGGACCAGATGATGCA
AAAGAAATTATGAGACACAGTTTCTTCTCTGGAGTAAACTGGCAAGATGTATATGATAAA
AAGCTTGTACCTCCTTTTAAACCTCAAGTAACATCTGAGACAGATACTAGATATTTTGAT
GAAGAATTTACAGCTCAGACTATTACAATAACACCACCTGAAAAATATGATGAGGATGGT
ATGGACTGCATGGACAATGAGAGGCGGCCGCATTTCCCTCAATTTTCCTACTCTGCAAGT
GGACGAGAATAAGTCTCTTTCATTCTGCTACTTCACTGTCATCTTCAATTTATTACTGAA
AATGATTCCTGGACATCACCAGTCCTAGCTCTTACACATAGCAGGGGCACCTTCCGACAT
CCCAGACCAGCCAAGGGTCCTCACCCCTCGCCACCTTTCACCCTCATGAAAACACACATA
CACGCAAATACACTCCAGTTTTTGTTTTTGCATGAAATTGTATCTCAGTCTAAGGTCTCA
TGCTGTTGCTGCTACTGTCTTACTATTA.

Amino acid sequences for Akt3 are also known in the art. See, for example, UniProtKB/Swiss-Prot accession no. Q9Y243 (Akt3_HUMAN), which is specifically incorporated by reference in its entirety and provides the following amino acid sequence:

(SEQ ID NO: 2)
MSDVTIVKEGWVQKRGEYIKNWRPRYFLLKTDGSFIGYKEKPQDVDLPY
PLNNFSVAKCQLMKTERPKPNTFIIRCLQWTTVIERTFHVDTPEEREEW
TEAIQAVADRLQRQEEERMNCSPTSQIDNIGEEEMDASTTHHKRKTMND
FDYLKLLGKGTFGKVILVREKASGKYYAMKILKKEVIIAKDEVAHTLTE
SRVLKNTRHPFLTSLKYSFQTKDRLCFVMEYVNGGELFFHLSRERVFSE
DRTRFYGAEIVSALDYLHSGKIVYRDLKLENLMLDKDGHIKITDFGLCK
EGITDAATMKTFCGTPEYLAPEVLEDNDYGRAVDWWGLGVVMYEMMCGR
LPFYNQDHEKLFELILMEDIKFPRTLSSDAKSLLSGLLIKDPNKRLGGG
PDDAKEIMRHSFFSGVNWQDVYDKKLVPPFKPQVTSETDTRYFDEEFTA
QTITITPPEKYDEDGMDCMDNERRPHFPQFSYSASGRE.

The domain structure of Akt3 is reviewed in Romano, Scientifica, Volume 2013 (2013), Article ID 317186, 12 pages, and includes an N-terminal pleckstrin homology domain (“PH”), followed by a catalytic kinase domain (“KD”), and the C-terminal regulatory hydrophobic region. The KD and regulatory domain are both important for the biological actions mediated by Akt protein kinases and exhibit the maximum degree of homology among the three Akt isoforms. The PH domain binds lipid substrates, such as phosphatidylinositol (3,4) diphosphate (“PIP2”) and phosphatidylinositol (3,4,5)triphosphate (“PIP3”). The ATP binding site is situated approximately in the middle of the catalytic kinase domain, which has a substantial degree of homology with the other components of the AGC kinases family, such as p70 S6 kinase (“S6K”) and p90 ribosomal S6 kinase (“RSK”), protein kinase A (“PKA”), and protein kinase B (“PKB”). The hydrophobic regulatory moiety is a typical feature of the AGC kinases family. With reference to SEQ ID NO:2, Akt 3 is generally considered to have the molecule processing and domain structure outlined as follows.

### Molecule Processing:

| Feature key | Position(s) | Length | Description |
|---|---|---|---|
| Initiator methionine | 1 | 1 | Removed |
| Chain | 2-479 | 478 | Akt3 |

### Regions:

| Feature key | Position(s) | Length | Description |
|---|---|---|---|
| Domain | 5-107 | 103 | PH |
| Domain | 148-405 | 258 | Protein kinase |
| Domain | 406-479 | 74 | AGC-kinase, C-terminal |
| Nucleotide binding | 154-162 | 9 | ATP |

### Sites:

| Feature key | Position(s) | Length | Description |
|---|---|---|---|
| Active site | 271 | 1 | Proton acceptor |
| Binding site | 177 | 1 | ATP |

The initiator methionine of SEQ ID NO:2 is disposable for Akt3 function. Therefore, in some embodiments, the compound directly or indirectly modulates expression or bioavailability of an Akt3 having the following amino acid sequence:

(SEQ ID NO: 3)
SDVTIVKEGWVQKRGEYIKNWRPRYFLLKTDGSFIGYKEKPQDVDLPYP
LNNFSVAKCQLMKTERPKPNTFIIRCLQWTTVIERTFHVDTPEEREEWT
EAIQAVADRLQRQEEERMNCSPTSQIDNIGEEEMDASTTHHKRKTMNDF
DYLKLLGKGTFGKVILVREKASGKYYAMKILKKEVIIAKDEVAHTLTES
RVLKNTRHPFLTSLKYSFQTKDRLCFVMEYVNGGELFFHLSRERVFSED
RTRFYGAEIVSALDYLHSGKIVYRDLKLENLMLDKDGHIKITDFGLCKE
GITDAATMKTFCGTPEYLAPEVLEDNDYGRAVDWWGLGVVMYEMMCGRL
PFYNQDHEKLFELILMEDIKFPRTLSSDAKSLLSGLLIKDPNKRLGGGP
DDAKEIMRHSFFSGVNWQDVYDKKLVPPFKPQVTSETDTRYFDEEFTAQ
TITITPPEKYDEDGMDCMDNERRPHFPQFSYSASGRE.

Two specific sites, one in the kinase domain (Thr-305 with reference to SEQ ID NO: 2) and the other in the C-terminal regulatory region (Ser-472 with reference to SEQ ID NO: 2), need to be phosphorylated for full activation of Akt3. Interaction between the PH domain of Akt3 and TCL1A enhances Akt3 phosphorylation and activation. IGF-1 leads to the activation of Akt3, which may play a role in regulating cell survival.

In some embodiments, a compound of Formula Ia, Ib, or Ic as described herein is an inhibitor of Akt3. In other embodiments, a compound of Formula Ia, Ib, or Ic as described herein is an activator of Akt3.

### Pharmaceutical Compositions

Some aspects of the invention involve administering an effective amount of a composition to a subject to achieve a specific outcome. The small molecule compositions useful according to the methods of the present invention thus can be formulated in any manner suitable for pharmaceutical use.

The formulations of the invention are administered in pharmaceutically-acceptable solutions, which may routinely contain pharmaceutically-acceptable concentrations of salt, buffering agents, preservatives, compatible carriers, adjuvants, and optionally other therapeutic ingredients.

For use in therapy, an effective amount of the compound can be administered to a subject by any mode allowing the compound to be taken up by the appropriate target cells. “Administering” the pharmaceutical composition of the present invention can be accomplished by any means known to the skilled artisan. Specific routes of administration include, but are not limited to, oral, transdermal (e.g., via a patch), parenteral injection (subcutaneous, intradermal, intramuscular, intravenous, intraperitoneal, intrathecal, etc.), or mucosal (intranasal, intratracheal, inhalation, intrarectal, intravaginal, etc.). An injection can be in a bolus or a continuous infusion.

For example the pharmaceutical compositions according to the invention are often administered by intravenous, intramuscular, or other parenteral means. They can also be administered by intranasal application, inhalation, topically, orally, or as implants; even rectal or vaginal use is possible. Suitable liquid or solid pharmaceutical preparation forms are, for example, aqueous or saline solutions for injection or inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro) capsules, suppositories, syrups, emulsions, suspensions, creams, drops, or preparations with protracted release of active compounds in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer R (1990) Science 249:1527-33.

The concentration of compounds included in compositions used in the methods of the invention can range from about 1 nM to about 100 μM. Effective doses are believed to range from about 10 picomole/kg to about 100 micromole/kg.

The pharmaceutical compositions are preferably prepared and administered in dose units. Liquid dose units are vials or ampoules for injection or other parenteral administration. Solid dose units are tablets, capsules, powders, and suppositories. For treatment of a patient, different doses may be necessary depending on activity of the compound, manner of administration, purpose of the administration (i.e., prophylactic or therapeutic), nature and severity of the disorder, age and body weight of the patient. The administration of a given dose can be carried out both by single administration in the form of an individual dose unit or else several smaller dose units. Repeated and multiple administration of doses at specific intervals of days, weeks, or months apart are also contemplated by the invention.

The compositions can be administered per se (neat) or in the form of a pharmaceutically-acceptable salt. When used in medicine the salts should be pharmaceutically acceptable, but non-pharmaceutically-acceptable salts can conveniently be used to prepare pharmaceutically-acceptable salts thereof. Such salts include, but are not limited to, those prepared from the following acids: hydrochloric, hydrobromic, sulphuric, nitric, phosphoric, maleic, acetic, salicylic, TsOH (p-toluene sulphonic acid), tartaric, citric, methane sulphonic, formic, malonic, succinic, naphthalene-2-sulphonic, and benzene sulphonic acids. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v); and thimerosal (0.004-0.02% w/v).

Compositions suitable for parenteral administration conveniently include sterile aqueous preparations, which can be isotonic with the blood of the recipient. Among the acceptable vehicles and solvents are water, Ringer's solution, phosphate buffered saline, and isotonic sodium chloride solution. In addition, sterile, fixed oils are conventionally employed as a solvent or suspending medium. For this purpose, any bland fixed mineral or non-mineral oil may be employed including synthetic mono- or diglycerides. In addition, fatty acids such as oleic acid find use in the preparation of injectables. Carrier formulations suitable for subcutaneous, intramuscular, intraperitoneal, intravenous, etc. administrations can be found in Remington's Pharmaceutical Sciences, Mack Publishing Company, Easton, PA.

The compounds useful in the invention can be delivered in mixtures of more than two such compounds. A mixture can further include one or more adjuvants in addition to the combination of compounds.

A variety of administration routes is available. The particular mode selected will depend, of course, upon the particular compound selected, the age and general health status of the subject, the particular condition being treated, and the dosage required for therapeutic efficacy. The methods of this invention can be practiced using any mode of administration that is medically acceptable, meaning any mode that produces effective levels of response without causing clinically unacceptable adverse effects. Preferred modes of administration are discussed above.

The compositions can conveniently be presented in unit dosage form and can be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product.

Other delivery systems can include time-release, delayed release, or sustained-release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids, or neutral fats such as mono-di- and tri-glycerides; hydrogel release systems; silastic systems; peptide-based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

### Methods of Treating Disease

In another aspect, a method of treating a disease in a subject in need thereof includes administering to the subject an effective amount of a compound of Formula Ia, Ib, or Ic as described herein.

In some embodiments, the disease is selected from the group consisting of neurodegenerative disease, cachexia, anorexia, obesity, obesity's complication, inflammatory disease, viral-induced inflammatory reaction, Gulf War Syndrome, tuberous sclerosis, retinitis pigmentosa, transplant rejection, cancer, an autoimmune disease, ischemic tissue injury, traumatic tissue injury, and a combination thereof.

In some embodiments, the compound of Formula Ia, Ib, or Ic modulates Akt3 in immune cells. Non-limiting examples of immune cells include T cells (e.g., T regulatory cells (“Tregs”)), B cells, macrophages, and glial cells (e.g., astrocytes, microglia, or oligodendrocytes). In some embodiments, the immune cells are Tregs. In some embodiments, the compound of Formula Ia, Ib, or Ic activates Akt3 signaling. In other embodiments, the compound of Formula Ia, Ib, or Ic inhibits Akt3 signaling. In some embodiments, the compound of Formula Ia, Ib, or Ic modulates Akt3 in Tregs. The inventors surprisingly found that, in some embodiments, the compound of Formula Ia, Ib, or Ic increases Treg activity or production while, in other embodiments, the compound decreases Treg activity or production. The inventors also surprisingly found that, in some embodiments, the compound of Formula Ia, Ib, or Ic activates Akt3 signaling while, in other embodiments, the compound inhibits Akt3 signaling.

### Neurodegenerative Disease

In some embodiments, a method of treating or preventing neurodegenerative diseases in a subject in need thereof is described, including modulating Akt3 signaling through administering to the subject an effective amount of a compound of Formula Ia, Ib, or Ic as described herein. In some embodiments, the neurodegenerative disease is selected from the group consisting of Parkinson's disease, Alzheimer's disease, amyotrophic lateral sclerosis, Motor Neuron Disease, Huntington's disease, HIV-induced neurodegeneration, Lewy Body Disease, spinal muscular atrophy, prion disease, spinocerebellar ataxia, familial amyloid polyneuropathy, multiple sclerosis, and a combination thereof.

Neurodegenerative diseases occur when nerve cells in the brain or peripheral nervous system lose function over time and ultimately die. In many of the neurodegenerative diseases, chronic neuroinflammation contributes to disease progression. Although current treatments may help relieve some of the physical or mental symptoms associated with neurodegenerative diseases, there are currently no ways to slow disease progression and no known cures.

While the mechanisms causing neurodegenerative processes are unknown, growing evidence suggests a critical role of immunity and the immune system in the pathogenesis of neurodegenerative diseases such as Alzheimer's disease, Parkinson's disease, Huntington's disease, multiple sclerosis, spinal muscular atrophy, familial amyloid polyneuropathy, and ALS. Tregs are a subset of CD4⁺ T cells that suppress immune responses and are essential mediators of self-tolerance and immune homeostasis (see Sakaguchi, et al., Cell, 133, 775-787 (2008)). Evidence suggest that Tregs play an important role in the progression of neurodegenerative diseases. For example, Akt3 can modulate the suppressive function of natural Tregs and the polarization of induced Tregs and, therefore, modulating Akt3 in immune cells can modulate immune responses. More specifically, activating Akt3 in immune cells can lead to increased immune suppressive responses, while inhibiting Akt3 in immune cells can lead to decreased immune suppressive responses. Without being bound by any one theory, it is believed that modulating Akt3 signaling in immune cells can be used for the treatment and prevention of neurodegenerative diseases.

In some embodiments, a method of treating or preventing neurodegenerative diseases in a subject in need thereof is described, including administering to the subject an Akt3 activator of a compound of Formula Ia, Ib, or Ic as described herein in an amount effective to induce an immune suppressive response and treat or delay the progression of the disease. In some embodiments, the Akt3 activator modulates an immune response by increasing a suppressive function of immune suppressive cells. In some embodiments, Akt3 is selectively activated in immune cells. Exemplary immune cells include, but are not limited to, T cells, B cells, macrophages, and glial cells, such as astrocytes, microglia, and oligodendrocytes. In a preferred embodiment, Akt3 is activated in Tregs. In some embodiments, the Akt3 activators can also be used to increase or promote the activity or production of Tregs, increase the production of cytokines, such as IL-10, from Tregs, increase the differentiation of Tregs, increase the number of Tregs, or increase the survival of Tregs.

In some embodiments, a method of treating or preventing neurodegenerative diseases in a subject in need thereof is described, including administering to the subject an Akt3 inhibitor of a compound of Formula Ia, Ib, or Ic as described herein in an amount effective to inhibit an immune suppressive response and treat or prevent the progression of the disease. In some embodiments, the Akt3 inhibitor of a compound of Formula Ia, Ib, or Ic as described herein modulates an immune response by decreasing an immune suppressive response or increasing an immune stimulatory response. In some embodiments, Akt3 is selectively inhibited in immune cells. Exemplary immune cells include but are not limited to T cells, B cells, macrophages, and glial cells, such as astrocytes, microglia, and oligodendrocytes. In a preferred embodiment, Akt3 is inhibited in Tregs.

In one embodiment, the compounds of Formula Ia, Ib, or Ic can treat or prevent ALS. ALS, also called Lou Gehrig's disease, is a progressive neurodegenerative disease that affects motor neurons in the brain and spinal cord. Symptoms of ALS include, but are not limited to, difficulty speaking, swallowing, walking, moving, and breathing. ALS usually affects men and women between the ages of 40 and 70. There are two different types of ALS, sporadic and familial. Sporadic, which is the most common form of the disease in the U.S., accounts for 90 to 95 percent of all cases. Familial ALS has been associated with mutations in Cu/Zn superoxide dismutase (SOD1). Oxidative stress, mitochondrial dysfunction, excitotoxicity, protein aggregation, endoplasmic reticulum stress, impairment of axonal transport, dysregulation of neuronal-glial interactions, and apoptosis have all been demonstrated to contribute to motor neuron injury in the presence of mutant SOD1. Without being bound by any one theory, it is believed that Treg dysfunction plays a role in the development of ALS and that administration of an Akt3 modulator can treat or prevent the progression of ALS. Some subjects with rapidly progressing ALS have a deficiency of the Treg master transcription factor FOXP3 which leads to impairment of Treg suppressive function. One embodiment provides a method of treating ALS in a subject in need thereof by administering an Akt3 activator to a subject in need thereof in an amount effective to activate Akt3 in immune cells and induce immune suppressive responses. In a preferred embodiment, Akt3 is activated in Tregs.

In some embodiments, administration of Akt3 activators of Formula Ia, Ib, or Ic as described herein to a subject having ALS slows disease progression and prolongs the subject's survival.

Other motor neuron diseases may be treated or prevented using the disclosed Akt3 modulators including, for example, progressive bulbar palsy, pseudobulbar palsy, primary lateral sclerosis, spinal muscular atrophy, and post-polio syndrome.

Parkinson's disease is a neurodegenerative disorder that predominantly affects dopamine-producing neurons in a specific area of the brain called substantia nigra. Parkinson's disease is a progressive disease that worsens over time as more neurons become impaired or die. The cause of neuronal death in Parkinson's is not known. Symptoms of Parkinson's disease include, but are not limited to, tremors in hands, arms, legs, jaw, or head, stiffness of the limbs and trunk, slowness of movement, and impaired balance and coordination.

One embodiment provides a method of treating Parkinson's disease by administering an Akt3 modulator to a subject in need thereof in an amount effective to activate or inhibit Akt3 in immune cells and induce an immune suppressive response. In some embodiments, administration of Akt3 activators to a subject having Parkinson's disease will slow or stop disease progression to unaffected areas of the brain.

In some embodiments, the disclosed Akt3 activators of Formula Ia, Ib, or Ic as described herein can be administered to a subject prophylactically if the subject has a family history of Parkinson's disease or other neurodegenerative diseases. In some embodiments, the Akt3 activators can protect neurons from disease induction or slow down the induction of the disease.

Huntington's disease is a progressive neurodegenerative disease. The disease is characterized by the progressive breakdown of nerve cells in the brain. Symptoms of Huntington's disease include, but are not limited to, involuntary movement problems and impairments in voluntary movement, such as involuntary jerking, muscle rigidity, slow or abnormal eye movements, impaired gait, posture, and balance, difficulty with the physical production of speech or swallowing; cognitive impairments, such as difficulty organizing, prioritizing, or focusing on tasks, lack of flexibility or the tendency to get stuck on a thought, behavior, or action, lack of impulse control, lack of awareness of one's own behaviors and abilities, slowness in processing thoughts or finding words, and difficulty in learning new information; and psychiatric disorders, such as depression. In one embodiment, the disclosed Akt3 modulators can lessen or slow the progression of symptoms of Huntington's disease.

One embodiment provides a method of treating Huntington's disease in a subject in need thereof by administering an Akt3 modulator to the subject in an amount effective to activate or inhibit Akt3 in immune cells and induce an immune suppressive response. In some embodiments, Akt3 modulators can slow down or stop the progression of disease symptoms in subjects with Huntington's disease. In another embodiments, Akt3 modulators can alter the Treg/Th17 balance.

Huntington's disease is largely genetic; every child of a parent with Huntington's disease has a 50/50 chance of inheriting the disease. In one embodiment, subjects with a familial history of Huntington's disease can be prophylactically administered one of the disclosed Akt3 modulators before symptoms of the disease appear to prevent or slow down the manifestation of disease symptoms.

Alzheimer's disease is a progressive disorder that causes brain cells to degenerate and eventually die. Alzheimer's disease is the most common cause of dementia and is hallmarked by a continuous decline in thinking, behavioral, and social skills that disrupts a person's ability to function independently. Symptoms of Alzheimer's disease include, but are not limited to, memory loss, impairment in thinking and reasoning abilities, difficulty in making judgments and decisions, and changes in personality and behavior. While the exact cause of Alzheimer's disease is not fully understood, it is believed that the core problem is dysfunctionality in brain proteins which disrupt neuronal function and unleash a series of toxic events. The damage most often starts in the region of the brain that controls memory, but the process begins years before the first symptoms. The loss of neurons spreads in a somewhat predictable pattern to other regions of the brain. By the late stage of the disease, the brain has shrunk significantly. Beta-amyloid plaques and tau protein tangles are most often attributed with the bulk of the damage and dysfunctionality of neurons in Alzheimer's disease.

One embodiment provides a method of treating Alzheimer's disease in a subject by administering an Akt3 activator to the subject in an amount effective to activate Akt3 in Tregs and activate downstream neuroprotective pathways in the brain. In another embodiment, subjects are administered an effective amount of an Akt3 activator to reduce or eliminate symptoms of Alzheimer's disease or to slow down disease progression.

Another embodiment provides a method of treating or preventing the progression of Alzheimer's disease in a subject by administering an Akt3 inhibitor of Formula Ia, Ib, or Ic as described herein to the subject in an amount effective to inhibit Akt3 in Tregs and induce an immune response or decrease an immune suppressive response. In some embodiments, inhibition of Akt3 in Tregs leads to beta-amyloid plaque clearance, mitigation of neuroinflammatory response, and reversal of cognitive decline.

Spinal muscular atrophy (“SMA”) is a group of chronic neuromuscular disorders that are characterized by progressive loss of motor neurons and muscle wasting. SMA is commonly classified in four types that vary in severity and the life stage during which the disease manifests. These types are:
SMA1 or Werdnig-Hoffmann disease, which manifests during age 0-6 months (“infantile” SMA);
SMA2 or Dubowitz disease, which manifests during age 6-18 months (“intermediate” SMA);
SMA3 or Kugelberg-Welander disease, which manifests after age 1 year (“juvenile” SMA); and
SMA4, which manifests during adulthood (“adult-onset” SMA).

The most severe form of SMA1 is sometimes termed SMA0 (“severe infantile” SMA). Signs and symptoms of SMA vary according to type, but the most common include, but are not limited to, limpness or tendency to flop, difficulty sitting, standing, or walking, loss of strength in respiratory muscles, twitching, and difficulty eating and swallowing. All types of SMA have been linked to exonal deletion and/or point mutations in the SMN1 gene, preventing expression of the SMN protein. Depending on the type, SMA can be treated with various gene therapies, assisted nutrition and respiration, orthopedics, and combinations thereof. Neuroprotective drugs are promising as a way to stabilize motor neuron loss, but currently available candidates have yet to successfully advance through clinical trials. Therefore, more candidate neuroprotective drugs are needed for treatment of SMA.

One embodiment provides a method of treating SMA in a subject by administering an Akt3 modulator of Formula Ia, Ib, or Ic as described herein to the subject in an amount effective to enable survival of motor neurons. In another embodiment, subjects are administered an effective amount of an Akt3 modulator to reduce or eliminate symptoms of SMA or to slow down disease progression.

Multiple sclerosis (“MS”) is a disease in which nerve cells in the brain and spinal cord become demyelinated, leading to nerve cell damage and disrupting signal transmission throughout the nervous system. Persons suffering MS can experience almost any neurological sign/symptom, with autonomic, visual, motor, and sensory impairment being most common. The precise cause of MS is unknown but is thought to be a combination of genetic, such as chromosomal aberrations in the major histocompatibility complex, and environmental factors, such as exposure to infectious agents and toxins. Treatments for MS, including, but not limited to, drugs and physical therapy, attempt to restore function in the affected area after an acute attack and prevent new attacks from occurring. There is no known cure for MS and many current drugs, while moderately effective, can have severe side effects and be poorly tolerated. Therefore, new drugs are needed for safe, effective restorative and preventative treatment of MS.

One embodiment provides a method of treating MS in a subject by administering an Akt3 modulator of Formula Ia, Ib, or Ic as described herein to the subject in an amount effective to restore loss of function after an attack and/or prevent attacks from occurring. In another embodiment, subjects are administered an effective amount of an Akt3 modulator to reduce or eliminate symptoms of MS or to slow down disease progression.

### Weight Loss

In some embodiments, a method of treating or preventing extreme weight loss is disclosed herein, including administering a compound disclosed here to a subject in need thereof. Non-limiting examples of weight loss disorders include cachexia, anorexia, and anorexia nervosa. An exemplary method includes inhibiting Akt3 in subjects in need thereof by administering a compound of Formula Ia, Ib, or Ic as described herein. Without being bound by any one theory, it is believed that Akt3 plays an important role in adipogenesis. White adipogenesis requires activation of a transcriptional cascade involving the sequential induction of a number of transcription factors including, but not limited to, FOXO1, several members of the C/EBP family, and PPARγ. FOXO1 is an essential negative regulator of adipogenesis and is primarily controlled through phosphorylation/acetylation on multiple residues by enzymes including Akt. FOXO1 can also be controlled by the serine/threonine protein kinase SGK1. SGK1 is downstream of PI3K and can inhibit FOXO1 upon phosphorylation. SGK1 is regulated by the serine/threonine protein kinase WNK1, which can also be regulated by Akt and SGK1. Akt3 suppresses adipogenesis through phosphorylation of WNK1, leading to downregulation of SGK1 activity and SGK-1-mediated inhibition of FOXO1. In one embodiment, inhibition of Akt3 in Tregs can promote adipogenesis and reverse disease-induced weight loss.

Cachexia, or wasting syndrome, is a multifactorial syndrome characterized by an ongoing loss of skeletal muscle that cannot be fully reversed by conventional nutritional support and leads to progressive functional impairment. Cachexia is so destructive that it taps into other sources of energy, namely skeletal muscle and adipose tissue, when the body senses lack of nutrition. It affects the majority of patients with advanced cancer and is associated with a reduction in ability to fight infection, treatment tolerance, response to therapy, quality of life, and duration of survival. In one embodiment, the cachexia is caused by a chronic disease such as, but not limited to, cancer, inflammatory disease, neurodegenerative disease, pathogenic infection, immunodeficiency disorder, weight gain disorder, weight loss disorder, hormone imbalance, tuberous sclerosis, retinitis pigmentosa, congestive heart failure, and a combination thereof. One embodiment provides a method of treating cachexia in a subject in need thereof by administering an Akt3 inhibitor of a compound of Formula Ia, Ib, or Ic as described herein to the subject in an amount effective to reduce symptoms of cachexia. Another embodiment provides a method of promoting weight gain in a subject in need thereof by administering an Akt3 inhibitor of a compound of Formula Ia, Ib, or Ic as described herein to the subject in an amount effective to promote adipogenesis in the subject. In one embodiment, a subject suspected of being susceptible for cachexia (for example, subjects who have been diagnosed with cancer or other diseases) can be prophylactically administered an Akt3 inhibitor to prevent or slow down the manifestation of cachexia syndrome. In some embodiments, the compound disclosed herein is used for treating cachexia by modulating Akt3 and not by modulating T regulatory cells.

Anorexia nervosa is an eating disorder characterized by weight loss or the lack of weight gain in growing children, difficulties maintaining an appropriate body weight for height, age, and stature, and, often, distorted body image. One of the first goals of treatment for anorexia is the restoration of a normal body weight. In some embodiments, the compound of Formula Ia, Ib, or Ic disclosed herein inhibits Akt3, which has been overactivated by estradiol, the levels of which are increased in subjects with anorexia. In some embodiments, the compound of Formula Ia, Ib, or Ic disclosed herein can be used to treat anorexia. In one embodiment, the disclosed Akt3 inhibitors of a compound of Formula Ia, Ib, or Ic can be administered to a subject diagnosed with anorexia in an amount effective to promote adipogenesis and reverse extreme weight loss.

### Obesity and Obesity's Complications

Diseases hallmarked by weight gain (e.g., obesity) are estimated to effect 40% of adults and 20% of children and adolescents in the United States alone, with those numbers trending upward. See “Overweight & Obesity: Data & Statistics”, U.S. Centers for Disease Control and Prevention, accessed Apr. 3, 2020. Obesity, which is characterized by a body mass index of >30 kg/m², increases the likelihood of various diseases (e.g., cardiovascular diseases and type 2 diabetes). Akt3 activation has been shown to be protective against obesity. In one embodiment, a method of treating obesity includes administering to a subject having obesity or at risk of developing obesity an Akt3 activator in an amount effective to reverse or prevent the effects of the disease.

In some embodiments, the compound disclosed herein modulating Akt3 is used for treating obesity and/or obesity's complications. In some embodiments, the obesity's complication is selected from the group consisting of glucose intolerance, hepatic steatosis, dyslipidemia, and a combination thereof. In some embodiments, the compound disclosed herein is used for treating Obesity and/or Obesity's complications by modulating Akt3 and not by modulating T regulatory cells.

### Inflammatory Diseases

Akt3 signaling has been linked to the chronic or acute inflammation that contributes to inflammatory diseases. One embodiment provides a method of treating or preventing an inflammatory disease in a subject in need thereof including administering to the subject a composition comprising an Akt3 modulator in an amount effective to modulate Akt3 signaling and treat or delay the progression of the disease. In some embodiments, the Akt3 modulator activates Akt3 signaling and/or increases Treg activity or production, resulting in an immunosuppressive effect.

Non-limiting examples of inflammatory disease include atopic dermatitis, allergy, asthma, and a combination thereof.

### Viral-Induced Inflammatory Reaction

Akt3 signaling has been linked to the acute immune responses that contribute to viral-induced inflammatory diseases, such as severe acute respiratory syndrome (“SARS”) and coronavirus disease 2019 (“COVID-19”). Therefore, in one embodiment, a method of treating a viral-induced inflammatory disease in a subject in need thereof includes administering to the subject an Akt3 modulator in an amount effective to reverse or slow down the progression of the disease.

### Cancer

In some embodiments, a method of treating or preventing cancer in a subject in need thereof is provided, including modulating Akt3 signaling through administering to the subject an effective amount of a compound of Formula Ia, Ib, or Ic as described herein. In some embodiments, the compound of Formula Ia, Ib, or Ic inhibits Akt3 signaling and/or decreases Treg activity or production, resulting in an immune response-activating effect.

In some embodiments, the cancer is selected from the group consisting of bladder cancer, brain cancer, breast cancer, cervical cancer, colorectal cancer, esophageal cancer, kidney cancer, liver cancer, lung cancer, nasopharyngeal cancer, pancreatic cancer, prostate cancer, skin cancer, stomach cancer, uterine cancer, ovarian cancer, testicular cancer, adult T-cell leukemia/lymphoma, and a combination thereof.

In some embodiments, the compounds and compositions disclosed herein are useful for treating leukemia. In some embodiments, the compounds and compositions disclosed herein that inhibit Akt3 are useful for treating leukemia. In these embodiments, the compounds and compositions disclosed herein that inhibit Akt3 are useful in vivo and ex vivo as immune response-stimulating therapeutics. The ability to inhibit Akt3 and thereby inhibit or reduce Treg-mediated immune suppression enables a more robust immune response. In some embodiments, the compounds and compositions disclosed herein are also useful to stimulate or enhance immune-stimulating or -activating responses involving T cells. In some embodiments, the compounds and compositions disclosed herein are useful for stimulating or enhancing an immune response in a host for treating leukemia by selectively inhibiting Akt3. In these embodiments, the compounds and compositions disclosed herein can be administered to a subject in an amount effective to stimulate T cells in the subject. The types of leukemia that can be treated with the compounds and compositions as disclosed herein include, but are not limited to, acute myeloid leukemia (AML), chronic myeloid leukemia (CML), acute lymphocytic leukemia (ALL), chronic lymphocytic leukemia (CLL), adult T-cell leukemia/lymphoma (ATLL) and chronic myelomonocytic leukemia (CMML).

In some embodiments, ATLL is almost exclusively diagnosed in adults, with a median age in the mid-60s. In some embodiments, there are four types of ATLL: (1) acute, (2) chronic, (3) smouldering, and (4) lymphomatous. In some embodiments, acute ATLL is the most common form, and is characterized by high white blood cell count, hypercalcemia, organomegaly, and high lactose dehydrogenase. In some embodiments, lymphomatous ATLL manifests in the lymph nodes with less than 1% circulating lymphocytes. In some embodiments, chronic and smouldering ATLL are characterized by a less aggressive clinical course and allow for long-term survival. In some embodiments, the four-year survival rate for acute and lymphomatous ATLL is less than 5%. In some embodiments, chronic and smouldering forms of ATLL have four-year survival rates of 26.9% and 62%, respectively. In some embodiments, the adult T-cell leukemia/lymphoma is caused by human T-cell lymphotropic virus (HTLV-1).

In some embodiments, the compounds and compositions disclosed herein are useful for treating ATLL. In some embodiments, the compounds and compositions disclosed herein that inhibit Akt3 are useful for treating ATLL. In some embodiments, Tregs expressing CD25 and FoxP3 may transform into ATLL cells. In some embodiments, ATLL cells display an activated helper/inducer T-cell phenotype but exhibit strong immunosuppressive activity. In some embodiments, the compounds and compositions disclosed herein that inhibit Akt3 reduce the immunosuppressive response of the ATLL cells. In other embodiments, the compounds and compositions disclosed herein that inhibit Akt3 increase an immune stimulatory response to overcome the strong immunosuppressive activity of ATLL cells.

In some embodiments, the compounds and compositions disclosed herein that are useful for treating leukemia or ATLL reduce or inhibit an immune suppressive response, such as, but not limited to an immune suppressive function of natural Treg (nTreg) cells and induction of conventional T cells into induced Treg (iTreg). In these embodiments, the immune suppressive function of nTreg cells that is reduced or inhibited is the secretion of one or more anti-inflammatory cytokines, such as, but not limited to IL10, TGFβ, or a combination thereof. In some embodiments, methods for treating leukemia or adult T-cell leukemia/lymphoma include administering to a subject a second active agent, such as, but not limited to, an anti-nausea drug, a chemotherapeutic drug, or a potentiating agent (e.g., cyclophosphamide).

### Autoimmune Disease

In some embodiments, the disease is an autoimmune disease. Non-limiting examples of autoimmune disease include achalasia, Addison's disease, adult Still's disease, agammaglobulinemia, alopecia areata, amyloidosis, ankylosing spondylitis, anti-glomerular basement membrane disease, anti-tubular basement membrane antibody nephritis, antiphospholipid syndrome, autoimmune angioedema, autoimmune dysautonomia, autoimmune encephalomyelitis, autoimmune hepatitis, autoimmune inner ear disease, autoimmune myocarditis, autoimmune oophoritis, autoimmune orchitis, autoimmune pancreatitis, autoimmune retinopathy, autoimmune urticaria, axonal and neuronal neuropathy, Baló disease, Behcet's disease, benign mucosal pemphigoid, bullous pemphigoid, Castleman disease, celiac disease, Chagas disease, chronic inflammatory demyelinating polyneuropathy, chronic recurrent multifocal osteomyelitis, Churg-Strauss syndrome, eosinophilic granulomatosis, cicatricial pemphigoid, Cogan's syndrome, cold agglutinin disease, congenital heart block, Coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura, pemphigoid gestationis, hidradenitis suppurativa (acne inversa), hypogammalglobulinemia, IgA nephropathy, IgG4-related sclerosing disease, immune thrombocytopenia purpura, inclusion body myositis, interstitial cystitis, juvenile arthritis, juvenile diabetes (type 1 diabetes), juvenile myositis, Kawasaki disease, Lambert-Eaton syndrome, leukocytoclastic vasculitis, lichen planus, lichen sclerosus, ligneous conjunctivitis, linear IgA disease, lupus, chronic Lyme disease, Meniere's disease, microscopic polyangiitis, mixed connective tissue disease, Mooren's ulcer, Mucha-Habermann disease, multifocal motor neuropathy, multiple sclerosis, myasthenia gravis, myositis, narcolepsy, neonatal lupus, neuromyelitis optica, neutropenia, ocular cicatricial pemphigoid, optic neuritis, palindromic rheumatism, pediatric autoimmune neuropsychiatric disorder, paraneoplastic cerebellar degeneration, paroxysmal nocturnal hemoglobinuria, Parry Romberg syndrome, pars planitis (peripheral uveitis), Parsonage-Turner syndrome, pemphigus, peripheral neuropathy, perivenous encephalomyelitis, pernicious anemia, POEMS syndrome, polyarteritis nodosa, polyglandular syndrome type I, polyglandular syndrome type II, polyglandular syndrome type III, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia, pyoderma gangrenosum, Raynaud's phenomenon, reactive arthritis, reflex sympathetic dystrophy, relapsing polychondritis, restless legs syndrome, retroperitoneal fibrosis, rheumatic fever, rheumatoid arthritis, sarcoidosis, Schmidt syndrome, scleritis, scleroderma, Sjögren's syndrome, sperm and testicular autoimmunity, stiff person syndrome, subacute bacterial endocarditis, Susac's syndrome, sympathetic ophthalmia, Takayasu's arteritis, temporal arteritis (giant cell arteritis), thrombocytopenia purpura, Tolosa-Hunt syndrome, transverse myelitis, ulcerative colitis, undifferentiated connective tissue disease, uveitis, vasculitis, vitiligo, and Vogt-Koyanagi-Harada disease.

### Other Indications

In some embodiments, a compound disclosed herein modulates Akt3 and is used for treating Gulf War Syndrome, tuberous sclerosis, retinitis pigmentosa, transplant rejection, ischemic tissue injury, or traumatic tissue injury. In some embodiments, the transplant rejection is Graft-versus-Host disease. In some embodiments, the compound disclosed herein is used for treating retinitis pigmentosa by modulating Akt3 and not by modulating T regulatory cells. In some embodiments, the compound disclosed herein is used for treating ischemic tissue injury or traumatic tissue injury. In some embodiments, the ischemic tissue injury or traumatic tissue injury is the ischemic tissue injury or traumatic tissue injury of the brain.

### Methods of Combination Therapy

In some embodiments, the disclosed compounds can be administered to a subject in need thereof alone or in combination with one or more additional therapeutic agents. In some embodiments, the compounds and the additional therapeutic agent are administered separately, but simultaneously. In some embodiments, the compound and the additional therapeutic agent are administered as part of the same composition. In other embodiments, the compound and the second therapeutic agent are administered separately and at different times, but as part of the same treatment regime.

In some embodiments, the subject can be administered a first therapeutic agent 1, 2, 3, 4, 5, 6, or more hours, or 1, 2, 3, 4, 5, 6, 7, or more days, before administration of a second therapeutic agent. In some embodiments, the subject can be administered one or more doses of the first agent every 1, 2, 3, 4, 5, 6 7, 14, 21, 28, 35, or 48 days prior to a first administration of second agent. The compounds disclosed herein can be the first or the second therapeutic agent.

In some embodiments, the compounds and the additional therapeutic agent can be administered as part of a therapeutic regimen. For example, if a first therapeutic agent can be administered to a subject every fourth day, the second therapeutic agent can be administered on the first, second, third, or fourth day, or combinations thereof. The first therapeutic agent or second therapeutic agent may be repeatedly administered throughout the entire treatment regimen.

Exemplary additional therapeutic agents include, but are not limited to, cytokines, chemotherapeutic agents, radionuclides, other immunotherapeutics, enzymes, antibiotics, antivirals (e.g., protease inhibitors alone or in combination with nucleosides for treatment of HIV or Hepatitis B or C), anti-parasites (e.g., helminths or protozoans), growth factors, growth inhibitors, hormones, hormone antagonists, antibodies and bioactive fragments thereof (including humanized, single chain, and chimeric antibodies), antigen and vaccine formulations (including adjuvants), peptide drugs, anti-inflammatories, ligands that bind to Toll-like receptors (including, but not limited to, CpG oligonucleotides) to activate the innate immune system, molecules that mobilize and optimize the adaptive immune system, other molecules that activate or up-regulate the action of cytotoxic T lymphocytes, NK cells and helper T-cells, and other molecules that deactivate or down-regulate suppressor or regulatory T-cells.

The additional therapeutic agents are selected based on the condition, disorder or disease to be treated. For example, the compounds of the invention can be co-administered with one or more additional agents that function to enhance or promote an immune response or reduce or inhibit an immune response.

### Chemotherapeutic Agents

In some embodiments, the compounds of the invention can be combined with one or more chemotherapeutic agents or pro-apoptotic agents. Representative chemotherapeutic agents include, but are not limited to, amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil, gemcitabine, hydroxycarbamide, idarubicin, ifosfamide, irinotecan, leucovorin, liposomal doxorubicin, liposomal daunorubicin, lomustine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, or a combination thereof. Representative pro-apoptotic agents include, but are not limited to fludarabinetaurosporine, cycloheximide, actinomycin D, lactosylceramide, 15d-PGJ(2), and combinations thereof.

### Anti-Inflammatories

Other suitable additional therapeutic agents include, but are not limited to, anti-inflammatory agents. In some embodiments, the anti-inflammatory agent can be non-steroidal, steroidal, or a combination thereof. One embodiment provides oral compositions containing about 1% (w/w) to about 5% (w/w), typically about 2.5% (w/w), of an anti-inflammatory agent. Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. In some embodiments, mixtures of these non-steroidal anti-inflammatory agents may also be employed.

Representative examples of steroidal anti-inflammatory drugs include, without limitation, corticosteroids, such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

### Immunosuppressive Agents

In some embodiments, the compound disclosed herein decreases Treg activity or production. In some embodiments, the compound disclosed herein is used in induction therapy for cancer. In some embodiments, the compound disclosed herein is used in combination with other immune therapeutic agents, immune modulators, costimulatory activating agonists, other cytokines and chemokines and factors, vaccines, oncolytic viruses, cell therapy, small molecules and targeted therapy, chemotherapy and radiation therapy. In some embodiments, the immune modulators include check point inhibitors such as anti-PD1, anti-CTLA4, anti-TIM3, anti-LAG3. In some embodiments, the costimulatory activating agonists including anti-OX40, anti-GITR, and the like. In some embodiments, the cell therapy includes engineered T cells, CAR-T, TCR-Tcells and others.

In some embodiments, the compound disclosed herein is used in combination with other immune therapeutic agents, immune modulators, biologics (e.g., antibodies), vaccines, small molecules and targeted therapy, anti-inflammatory, cell therapy (e.g., engineered Tregs and other type of cells, chemotherapy and radiation therapy.

In some embodiments, the compound disclosed herein, either used alone or in combination with other agents, is administered in vivo to a patient by intravenous, intramuscular, or other parenteral means. They can also be administered by intranasal application, inhalation, rectally, vaginally, topically, orally, or as implants. In other embodiments, the compound disclosed herein, either used alone or in combination with other agents, is applied ex vivo to enhance the function of suppressive Tregs, including natural tregs, induce-Tregs, engineered Tregs and other type of suppressive T cells, which optionally can then be used to treat a patient.

In some embodiments, the additional therapeutic agent is an immune suppressant. Immunosuppressive agents include, but are not limited to, antibodies against other lymphocyte surface markers (e.g., CD40, alpha-4 integrin) or against cytokines, fusion proteins (e.g., CTLA-4-Ig (Orencia®), TNFR-Ig (Enbrel®)), TNF-α blockers, such as Enbrel, Remicade, Cimzia, and Humira, cyclophosphamide (“CTX”) (e.g., Endoxan®, Cytoxan®, Neosar®, Procytox®, and Revimmune™), methotrexate (“MTX”) (e.g. Rheumatrex® and Trexall®), belimumab (e.g. Benlysta®), other immunosuppressive drugs (e.g., cyclosporin A, FK506-like compounds, rapamycin compounds, and steroids), anti-proliferatives, cytotoxic agents, and other compounds that may assist in immunosuppression.

In some embodiments, the additional therapeutic agent can be a checkpoint inhibitor. In some embodiments, the additional therapeutic agent can be a CTLA-4 fusion protein, such as CTLA-4-Ig (abatacept). CTLA-4-Ig fusion proteins can compete with the co-stimulatory receptor, CD28, on T-cells for binding to CD80/CD86 (B7-1/B7-2) on antigen presenting cells, and thus function to inhibit T-cell activation. In another embodiment, the additional therapeutic agent is a CTLA-4-Ig fusion protein known as belatacept. Belatacept contains two amino acid substitutions (L104E and A29Y) that can markedly increase its avidity to CD86 in vivo. In another embodiment, the additional therapeutic agent is Maxy-4.

In another embodiment, the additional therapeutic agent is CTX. CTX (the generic name for Endoxan®, Cytoxan®, Neosar®, Procytox®, and Revimmune™), also known as cytophosphane, is a nitrogen mustard alkylating agent from the oxazophorines group. It can be used to treat various types of cancer and some autoimmune disorders. CTX is the primary drug used for diffuse proliferative glomerulonephritis in patients with renal lupus.

In some embodiments, the additional therapeutic agent can be administered in an effective amount to reduce the blood or serum levels of anti-double-stranded DNA (“anti-ds DNA”) auto antibodies and/or to reduce proteinuria in a patient in need thereof.

In another embodiment, the additional therapeutic agent can increase the amount of adenosine in the serum (see, for example, WO 08/147482). For example, the second therapeutic agent can be CD73-Ig, recombinant CD73, or another agent (e.g., a cytokine, monoclonal antibody, or small molecule) that increases the expression of CD73 (see, for example WO 04/084933). In another embodiment, the additional therapeutic agent is Interferon-beta.

In some embodiments, the additional therapeutic agent can be a small molecule that inhibits or reduces differentiation, proliferation, activity, cytokine production, and/or cytokine secretion by Th1, Th17, Th22, and/or other cells that secrete, or cause other cells to secrete, inflammatory molecules, including, but not limited to, IL-1β, TNF-α, TGF-beta, IFN-γ, IL-18 IL-17, IL-6, IL-23, IL-22, IL-21, and MMPs. In another embodiment, the additional therapeutic agent is a small molecule that interacts with Tregs, enhances Treg activity, promotes or enhances IL-10 secretion by Tregs, increases the number of Tregs, increases the suppressive capacity of Tregs, or combinations thereof.

In some embodiments, the composition increases Treg activity or production. Exemplary Treg enhancing agents include, but are not limited to, glucocorticoid fluticasone, salmeteroal, antibodies to IL-12, IFN-γ, and IL-4; vitamin D3, and dexamethasone, and combinations thereof.

In some embodiments, the additional therapeutic agent is an antibody, for example, a function-blocking antibody against a proinflammatory molecule such as IL-6, IL-23, IL-22, or IL-21.

In some embodiments, the additional therapeutic agent includes a nucleic acid. In some embodiments, the additional therapeutic agent includes a ribonucleic acid.

### Combination Treatments for Neurodegenerative Diseases

In some embodiments, the compounds disclosed herein can be administered with a second therapeutic that is selected based on the subject's disease state. In some embodiments, the second therapeutic can be a treatment for Alzheimer's disease. Current treatments for Alzheimer's disease include, but are not limited to, cholinesterase inhibitors, such as donepezil, rivastigmine, and galantamine; memantine; antidepressants, such as citalopram, fluoxetine, paroxetine, sertraline, and trazadone; anxiolytics, such as lorazepam and oxazepam; and antipsychotics, such as aripiprazole, clozapine, haloperidol, olanzapine, quetiapine, risperidone, and ziprasidone.

In another embodiment, the additional therapeutic agent can be a treatment for ALS. There are currently two U.S. FDA-approved treatments for ALS: riluzole and edavarone. Both drugs have been shown to slow down the progression of ALS. In addition to riluzole and edavarone, subjects with ALS can also be treated with drugs that target a specific symptom of the disease. Exemplary such drugs include, but are not limited to, drugs to reduce spasticity such, as antispastics (e.g., baclofen, dantrolene, and diazepam); drugs to help control nerve pain, such as amitriptyline, carbamazepine, duloxetine, gabapentin, lamotrigine, milnacipran, nortriptyline, pregabalin and venlafaxine; and drugs to help patients swallow, such as trihexyphenidyl or amitriptyline.

In one embodiment, the additional therapeutic agent can be a treatment for Parkinson's disease. Current treatments for Parkinson's disease include, but are not limited to, carbidopa-levodopa; dopamine agonists, such as pramipexole, ropinirole, and rotigotine; MAO B inhibitors, such as selegiline, rasagiline, and safinamide; catechol O-methyltransferase inhibitors, such as entacapone and tolcapone; anticholinergics, such as bentztropine and trihexyphenidyl; and amantadine.

In some embodiments, the second therapeutic agent can be a treatment for Huntington's disease. Current treatments for Huntington's disease include, but are not limited to, tetrabenazine; antipsychotics, such as haloperidol, chlorpromazine, risperidone, and quetiapine; amantadine; levetiracetam; clonazepam; antidepressants, such as citalopram, escitalopram, fluoxetine, and sertraline; and anticonvulsants, such as valproate, carbamazepine, and lamotrigine.

### Combination Treatments for Weight Loss

In some embodiments, the compounds disclosed herein can be administered to a subject with an additional therapeutic agent that is used to treat cachexia or extreme weight loss. The current strategy for treating cachexia and extreme weight loss is to improve appetite by using appetite stimulants to ensure adequate intake of nutrients. Pharmacological interventions with appetite stimulants, nutrient supplementation, 5-HT3 antagonists, and Cox-2 inhibitor have been used to treat cancer cachexia.

In some embodiments, appetite stimulants are, for example, vitamins, minerals, or herbs including, but not limited to, zinc, thiamine, or fish oil. In another embodiment, the appetite stimulant is a medication including, but not limited to, dronabinol, megesterol, and oxandrolone.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification, and guidance which can be adapted to the practice of this invention in its various embodiments and equivalents thereof.

### EXAMPLES

### Example 1: Compound 1 (3-((6-nitroquinolin-4-yl)amino)-N-(3-(pyridin-4-ylamino)phenyl)benzamide)

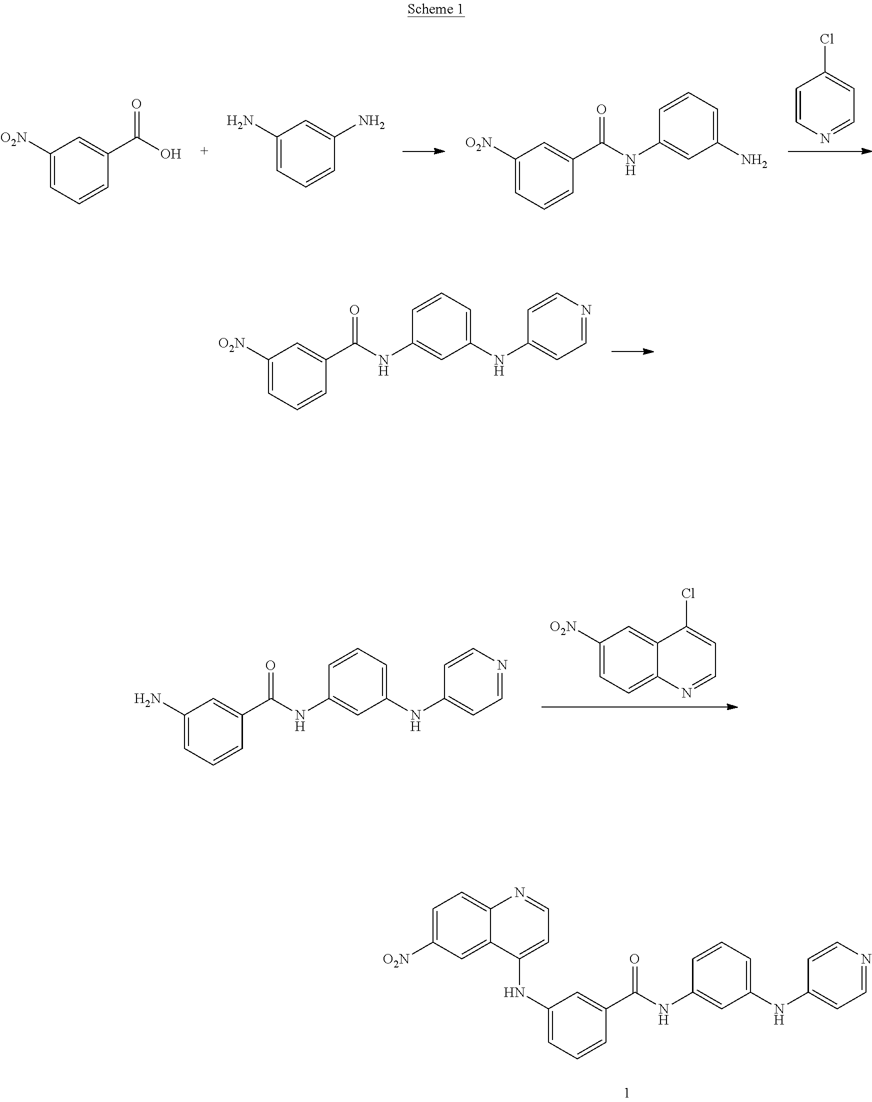

As shown in Scheme 1, meta-nitrobenzoic acid was coupled with 1,3-phenylenediamine using EDCI in the presence of HOBt and DIPEA. The resulting intermediate was coupled with 4-chloro-pyridine followed by reduction of the nitro group into an amino group using Sn/HCl. The resulting amino-intermediate was then reacted with 4-chloro-6-nitro-quinoline in EtOH under reflux for 3 hours with the addition of 2-3 drops of TEA to give meta-substituted product Compound 1. The final product was precipitated from the reaction mixture soon after it reached room temperature and then filtered off and purified via recrystallization from EtOH diethyl ether 1:1.

The compounds shown in the following examples were made in an analogous manner based on the experimental procedure described in Example 1, and/or as described below, and/or by a method known in the art.

The following abbreviations used in the following examples have the following definitions: AIBN=azobisisobutyronitrile; DCE=dichloroethane; DCM=dichloromethane; DIEPA or DIPEA=N,N-diisopropylethylamine; DMAP=4-dimethylaminopyridine; DMF=dimethylformamide; EA or EtOAc=ethyl acetate; EDC or EDCI=1-ethyl-3-(3-dimethylaminopropyl) carbodiimide; HATU=1-[bis(dimethylamino)methylene]-1H-1,2,3-triazolo[4,5-b]pyridinium 3-oxid hexafluorophosphate; HPLC=high-performance liquid chromatography; LCMS=liquid chromatography mass spectrometry; MS=mass spectrometry; NBS=N-bromosuccinimide; NMR=nuclear magnetic resonance; PE=petroleum ether; RT=retention time (e.g., HPLC retention time); TEA=triethylamine; TFA=trifluoroacetic acid; THF=tetrahydrofuran; TLC=thin layer chromatography; and TsOH or TosOH=p-toluenesulfonic acid. These abbreviations and definitions are not intended to be limiting of other abbreviations and definitions in the application.

In one or more of the following examples, the following general methods were used. Substrates and reagents were commercially available and used without further purification. The reaction was monitored by LCMS or TLC using pre-coated glass plates. Column chromatography was performed using silica gel (200-300 mesh) or a Biotage machine (normal HPLC). The prep-HPLC method used a Gilson 281 (PHG012) instrument, a Welch 10 μm 150A 21.2*250 mm column, a mobile phase consisting of A: water (12 mM NH₄HCO₃), B: acetonitrile, a flow rate of 30.00 mL/minute, and detection at 214/254 nm. ¹H NMR spectra were recorded in CDCl₃/MeOD/DMSO-_{d6} on 500 or 400 MHz Bruker NMR spectrometer and resonances are given in parts per million relative to tetramethylsilane. Data are reported as follows: chemical shift, multiplicity (s=singlet, d=doublet, t=triplet, m=multiplet), coupling constants (Hz), and integration. MS data were obtained on a LCMS machine equipped with an electrospray source.

### Example 2: Compound 2 (3-((6-cyanoquinolin-4-yl)amino)-N-(4-(pyridin-4-ylamino)phenyl)benzamide)

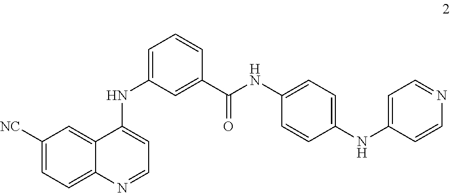 [SMILES: N#Cc1ccc2nccc(Nc3cccc(C(=O)Nc4ccc(Nc5ccncc5)cc4)c3)c2c1]

Compound 2 was prepared by a method known in the art and/or a method analogous to those described herein. Compound 2 (3-((6-cyanoquinolin-4-yl)amino)-N-(4-(pyridin-4-ylamino)phenyl)benzamide): C₂₈H₂₀N₆O; 456.51 g/mol; 26 mg; yellow solid; ESI-LCMS m/z=457 [M+H]⁺; LCMS RT=1.594 min, 100% (214 nm).

### Example 3: Compound 3 (3-((6-fluoroquinolin-4-yl)amino)-N-(4-(pyridin-4-ylamino)phenyl)benzamide)

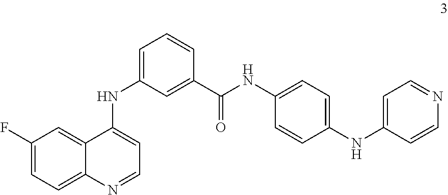 [SMILES: O=C(Nc1ccc(Nc2ccncc2)cc1)c1cccc(Nc2ccnc3ccc(F)cc23)c1]

Compound 3 was prepared by a method known in the art and/or a method analogous to those described herein. Compound 3 (3-((6-fluoroquinolin-4-yl)amino)-N-(4-(pyridin-4-ylamino)phenyl)benzamide): C₂₇H₂₀FN₅₀; 449.49 g/mol; 34 mg; white solid; ESI-LCMS m/z=450 [M+H]⁺; LCMS RT=1.614 min, 99% (214 nm).

### Example 4: Compound 4 (4-((3-(6-(pyridin-4-ylamino)-1H-benzo[d]imidazol-2-yl)phenyl)amino)quinoline-6-carbonitrile)

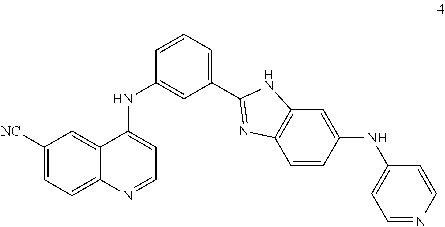 [SMILES: N#Cc1ccc2nccc(Nc3cccc(-c4nc5ccc(Nc6ccncc6)cc5[nH]4)c3)c2c1]

Compound 4 was prepared by a method known in the art and/or a method analogous to those described herein. Compound 4 (4-((3-(6-(pyridin-4-ylamino)-1H-benzo[d]imidazol-2-yl)phenyl)amino)quinoline-6-carbonitrile): C₂₈H₁₉N₇; 453.51 g/mol; 22 mg; yellow solid; ESI-LCMS m/z=454 [M+H]⁺; LCMS RT=1.634 min, 100% (214 nm).

### Example 5: Compound 5 (3-((6-fluoroquinolin-4-yl)amino)-N-(4-(pyridin-4-yloxy)phenyl)benzamide)

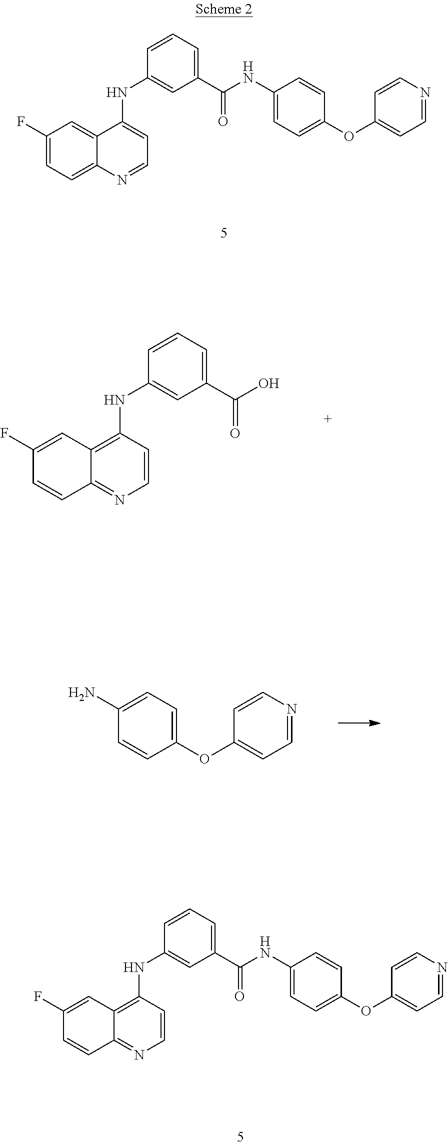 [SMILES: O=C(Nc1ccc(Oc2ccncc2)cc1)c1cccc(Nc2ccnc3ccc(F)cc23)c1 ; O=C(O)c1cccc(Nc2ccnc3ccc(F)cc23)c1 ; Nc1ccc(Oc2ccncc2)cc1 ; O=C(Nc1ccc(Oc2ccncc2)cc1)c1cccc(Nc2ccnc3ccc(F)cc23)c1]

Compound 5 was prepared by the method shown in Scheme 2. Compound 5 (3-((6-fluoroquinolin-4-yl)amino)-N-(4-(pyridin-4-yloxy)phenyl)benzamide) was prepared as shown in Scheme 2: C₂₇H₁₉FN₄O₂; 450.47 g/mol; 13 mg; white solid; ESI-LCMS m/z=451 [M+H]⁺; LCMS RT=0.99 min, >95.00% (214 nm).

### Example 6: Compound 6 (3-((6-fluoroquinolin-4-yl)amino)-N-(4-((2-methylpyridin-4-yl)oxy)phenyl)benzamide)

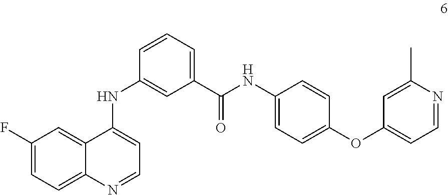 [SMILES: Cc1cc(Oc2ccc(NC(=O)c3cccc(Nc4ccnc5ccc(F)cc45)c3)cc2)ccn1]

Compound 6 was prepared by a method known in the art and/or a method analogous to those described herein. Compound 6 (3-((6-fluoroquinolin-4-yl)amino)-N-(4-((2-methylpyridin-4-yl)oxy)phenyl)benzamide): C₂₈H₂₁FN₄O₂; 464.50 g/mol; 18 mg; pale yellow solid; ESI-LCMS m/z=456 [M+H]⁺; LCMS RT=1.43 min, >95.00% (214 nm).

### Example 7: Compound 7 (4-((6-fluoroquinolin-4-yl)amino)-N-(3-phenoxyphenyl)benzamide)

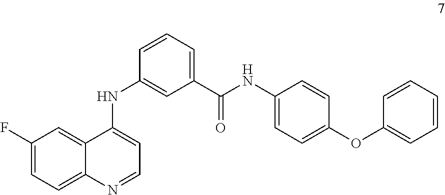 [SMILES: O=C(Nc1ccc(Oc2ccccc2)cc1)c1cccc(Nc2ccnc3ccc(F)cc23)c1]

Compound 7 was prepared by a method known in the art and/or a method analogous to those described herein. Compound 7 (4-((6-fluoroquinolin-4-yl)amino)-N-(3-phenoxyphenyl)benzamide): C₂₈H₂₀FN₃O₂; 449.49 g/mol; 13 mg; white solid; ESI-LCMS m/z=450 [M+H]⁺; LCMS RT=1.74 min, >95.00% (214 nm).

### Example 8: Compound 8 (3-(pyridin-4-ylamino)-N-(4-(pyridin-4-ylamino)phenyl)benzamide)

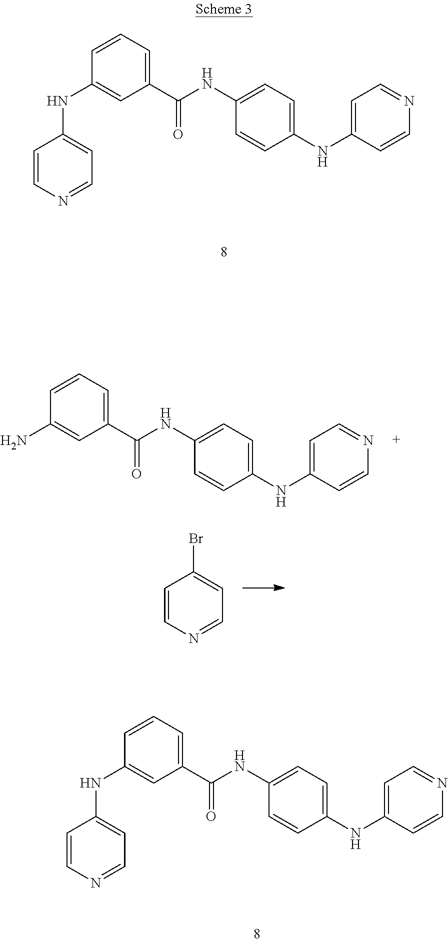 [SMILES: O=C(Nc1ccc(Nc2ccncc2)cc1)c1cccc(Nc2ccncc2)c1 ; Nc1cccc(C(=O)Nc2ccc(Nc3ccncc3)cc2)c1 ; Brc1ccncc1 ; O=C(Nc1ccc(Nc2ccncc2)cc1)c1cccc(Nc2ccncc2)c1]

Compound 8 was prepared by the method shown in Scheme 3. Compound 8 (3-(pyridin-4-ylamino)-N-(4-(pyridin-4-ylamino)phenyl)benzamide) was prepared as shown in Scheme 3: C₂₃H₁₉N₅O; 381.44 g/mol; 12 mg; pale yellow solid; ESI-LCMS m/z=382 [M+H]⁺; LCMS RT=1.30 min, >95.00% (214 nm).

### Example 9: Compound 9 (N-(4-(pyridin-4-ylamino)phenyl)-3-(quinolin-4-ylamino)benzamide)

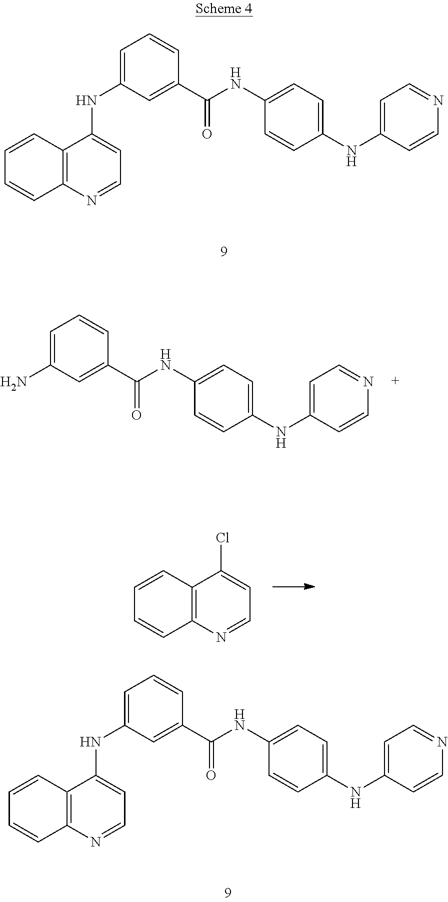

Compound 9 was prepared by the method shown in Scheme 4. Compound 9 (N-(4-(pyridin-4-ylamino)phenyl)-3-(quinolin-4-ylamino)benzamide) was prepared as shown in Scheme 4: C₂₇H₂₁N₅O; 431.50 g/mol; 24 mg; pale yellow solid; ESI-LCMS m/z=432 [M+H]⁺; LCMS RT=1.46 min, >95.00% (214 nm).

### Example 10: Compound 10 (N-(4-(pyridin-4-yloxy)phenyl)-3-(quinolin-4-ylamino)benzamide)

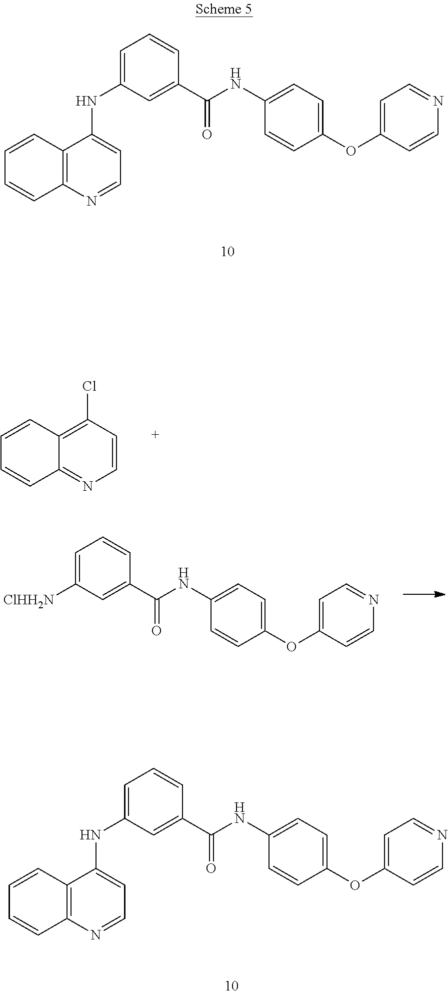

Compound 10 was prepared by the method shown in Scheme 5. Compound 10 (N-(4-(pyridin-4-yloxy)phenyl)-3-(quinolin-4-ylamino)benzamide) was prepared as shown in Scheme 5: C₂₇H₂₀N₄O₂; 432.48 g/mol; 29 mg; pale yellow solid; ESI-LCMS m/z=433 [M+H]⁺; LCMS RT=1.43 min, >95.00% (214 nm).

### Example 11: Compound 11 (3-((2-methylpyridin-4-yl)amino)-N-(4-(pyridin-4-ylamino)phenyl)benzamide)

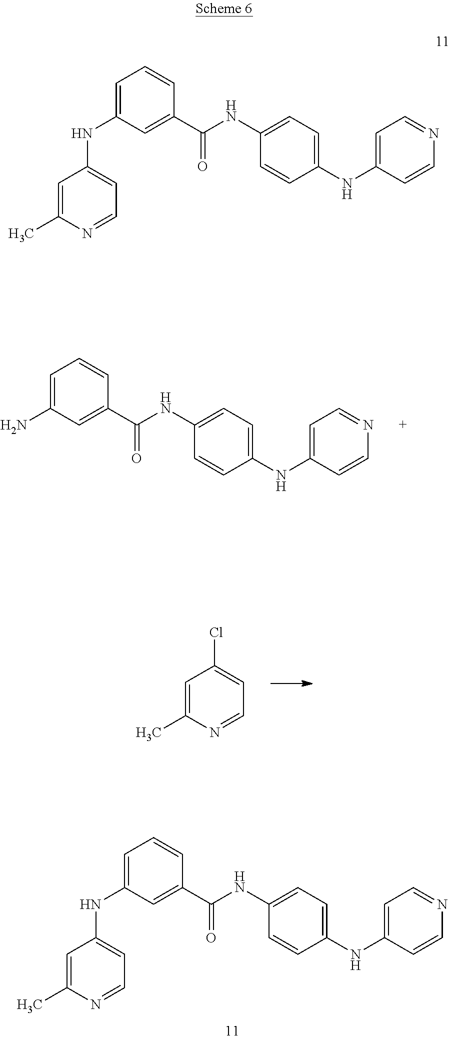

Compound 11 was prepared by the method shown in Scheme 6. Compound 11 (3-((2-methylpyridin-4-yl)amino)-N-(4-(pyridin-4-ylamino)phenyl)benzamide) was prepared as shown in Scheme 6: C₂₄H₂₁N₅O; 395.47 g/mol; 16 mg; pale yellow solid; ESI-LCMS m/z=396 [M+H]⁺; LCMS RT=1.32 min, >95.00% (214 nm).

### Example 12: Compound 12 (3-((3-methylquinolin-4-yl)amino)-N-(4-(pyridin-4-ylamino)phenyl)benzamide)

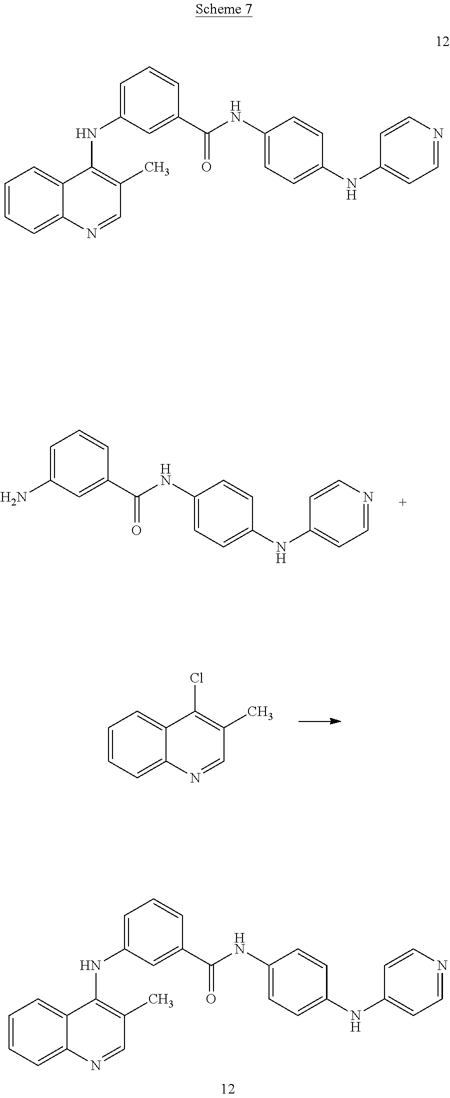 [SMILES: Cc1cnc2ccccc2c1Nc1cccc(C(=O)Nc2ccc(Nc3ccncc3)cc2)c1 ; Nc1cccc(C(=O)Nc2ccc(Nc3ccncc3)cc2)c1 ; Cc1cnc2ccccc2c1Cl ; Cc1cnc2ccccc2c1Nc1cccc(C(=O)Nc2ccc(Nc3ccncc3)cc2)c1]

Compound 12 was prepared by the method shown in Scheme 7. Compound 12 (3-((3-methylquinolin-4-yl)amino)-N-(4-(pyridin-4-ylamino)phenyl)benzamide) was prepared as shown in Scheme 7: C₂₈H₂₃N₅O; 445.53 g/mol; 19 mg; pale yellow solid; ESI-LCMS m/z=446 [M+H]⁺; LCMS RT=1.48 min, >95.00% (214 nm).

### Example 13: Compound 13 (3-((2-methylquinolin-4-yl)amino)-N-(4-(pyridin-4-ylamino)phenyl)benzamide)

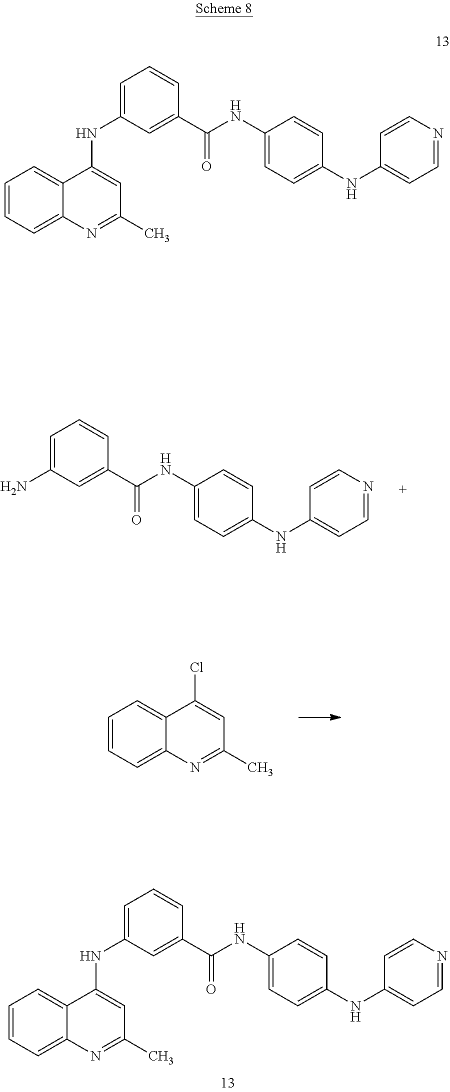 [SMILES: Cc1cc(Nc2cccc(C(=O)Nc3ccc(Nc4ccncc4)cc3)c2)c2ccccc2n1 ; Nc1cccc(C(=O)Nc2ccc(Nc3ccncc3)cc2)c1 ; Cc1cc(Cl)c2ccccc2n1 ; Cc1cc(Nc2cccc(C(=O)Nc3ccc(Nc4ccncc4)cc3)c2)c2ccccc2n1]

Compound 13 was prepared by the method shown in Scheme 8. Compound 13 (3-((2-methylquinolin-4-yl)amino)-N-(4-(pyridin-4-ylamino)phenyl)benzamide) was prepared as shown in Scheme 8: C₂₈H₂₃NO; 445.53 g/mol; 20 mg; pale yellow solid; ESI-LCMS m/z=446 [M+H]⁺; LCMS RT=1.46 min, >95.00% (214 nm).

### Example 14: Compound 14 (3-((8-methylquinolin-4-yl)amino)-N-(4-(pyridin-4-ylamino)phenyl)benzamide)

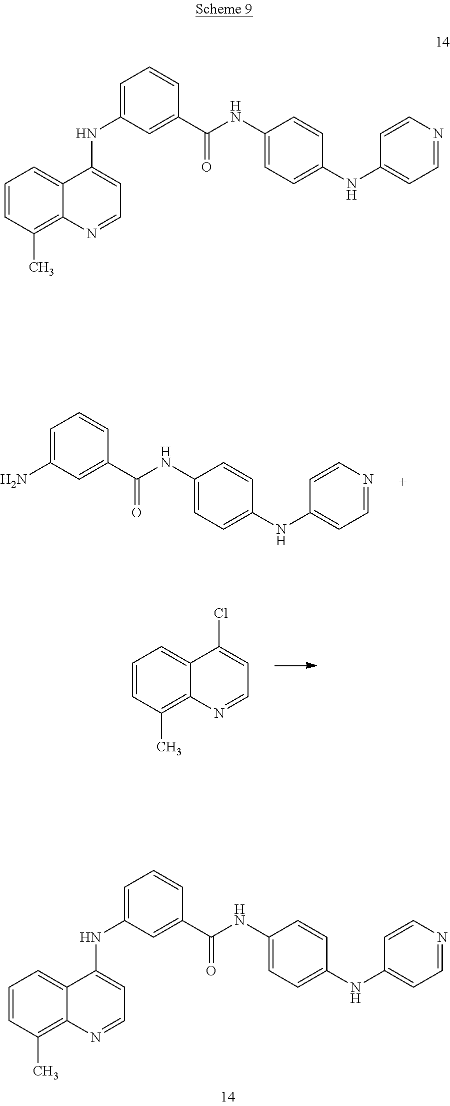 [SMILES: Cc1cccc2c(Nc3cccc(C(=O)Nc4ccc(Nc5ccncc5)cc4)c3)ccnc12 ; Nc1cccc(C(=O)Nc2ccc(Nc3ccncc3)cc2)c1 ; Cc1cccc2c(Cl)ccnc12 ; Cc1cccc2c(Nc3cccc(C(=O)Nc4ccc(Nc5ccncc5)cc4)c3)ccnc12]

Compound 14 was prepared by the method shown in Scheme 9. Compound 14 (3-((8-methylquinolin-4-yl)amino)-N-(4-(pyridin-4-ylamino)phenyl)benzamide) was prepared as shown in Scheme 9: C₂₈H₂₃N₅O; 445.53 g/mol; 29 mg; pale yellow solid; ESI-LCMS m/z=446 [M+H]⁺; LCMS RT=1.54 min, >95.00% (214 nm).

### Example 15: Compound 15 (3-((7-methylquinolin-4-yl)amino)-N-(4-(pyridin-4-ylamino)phenyl)benzamide)

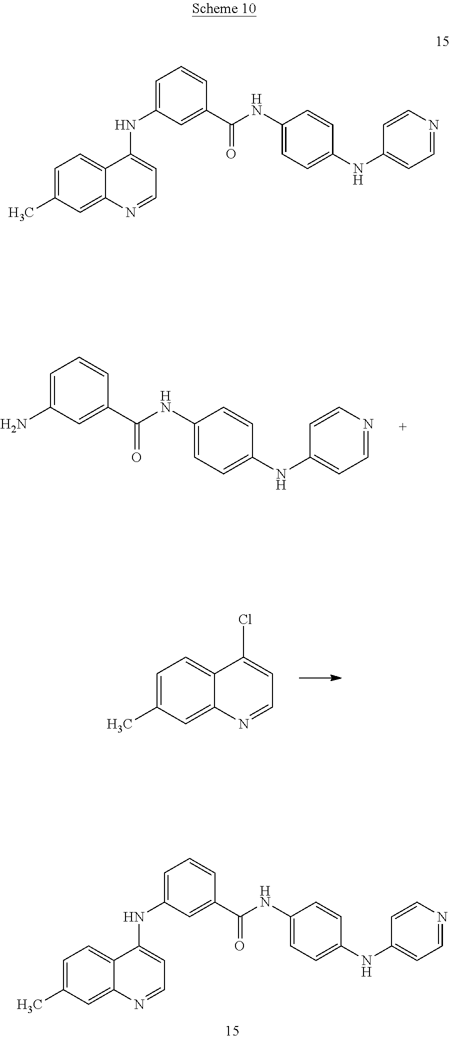 [SMILES: Cc1ccc2c(Nc3cccc(C(=O)Nc4ccc(Nc5ccncc5)cc4)c3)ccnc2c1 ; Nc1cccc(C(=O)Nc2ccc(Nc3ccncc3)cc2)c1 ; Cc1ccc2c(Cl)ccnc2c1 ; Cc1ccc2c(Nc3cccc(C(=O)Nc4ccc(Nc5ccncc5)cc4)c3)ccnc2c1]

Compound 15 was prepared by the method shown in Scheme 10. Compound 15 (3-((7-methylquinolin-4-yl)amino)-N-(4-(pyridin-4-ylamino)phenyl)benzamide) was prepared as shown in Scheme 10: C₂₈H₂₃N₅O; 445.53 g/mol; 24 mg; pale yellow solid; ESI-LCMS m/z=446 [M+H]⁺; LCMS RT=1.48 min, >95.00% (214 nm).

### Example 16: Compound 16 (3-((5-methylquinolin-4-yl)amino)-N-(4-(pyridin-4-ylamino)phenyl)benzamide)

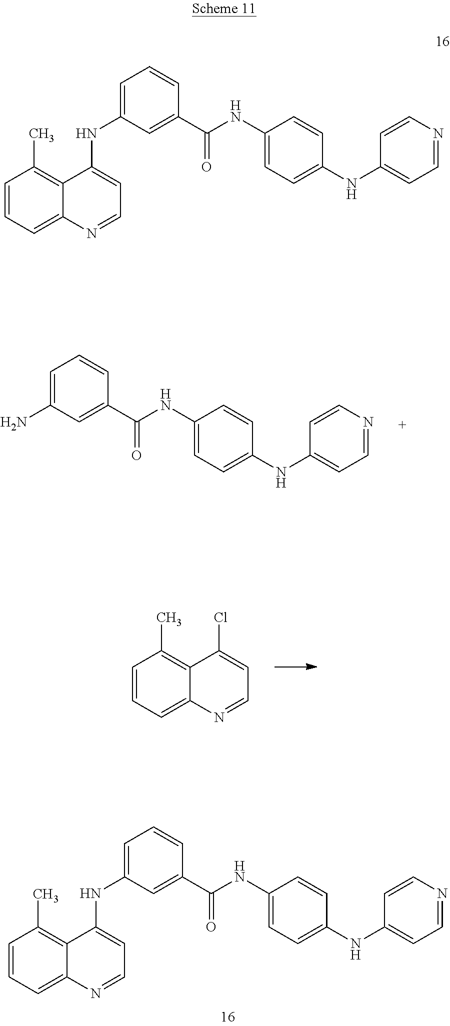 [SMILES: Cc1cccc2nccc(Nc3cccc(C(=O)Nc4ccc(Nc5ccncc5)cc4)c3)c12 ; Nc1cccc(C(=O)Nc2ccc(Nc3ccncc3)cc2)c1 ; Cc1cccc2nccc(Cl)c12 ; Cc1cccc2nccc(Nc3cccc(C(=O)Nc4ccc(Nc5ccncc5)cc4)c3)c12]

Compound 16 was prepared by the method shown in Scheme 11. Compound 16 (3-((5-methylquinolin-4-yl)amino)-N-(4-(pyridin-4-ylamino)phenyl)benzamide) was prepared as shown in Scheme 11: C₂₈H₂₃N₅O; 445.53 g/mol; 17 mg; pale yellow solid; ESI-LCMS m/z=446 [M+H]⁺; LCMS RT=1.49 min, >95.00% (214 nm).

### Example 17: Compound 17 (4-((3-(5-(pyridin-4-ylamino)-3H-imidazo[4,5-b]pyridin-2-yl)phenyl)amino)quinoline-6-carbonitrile)

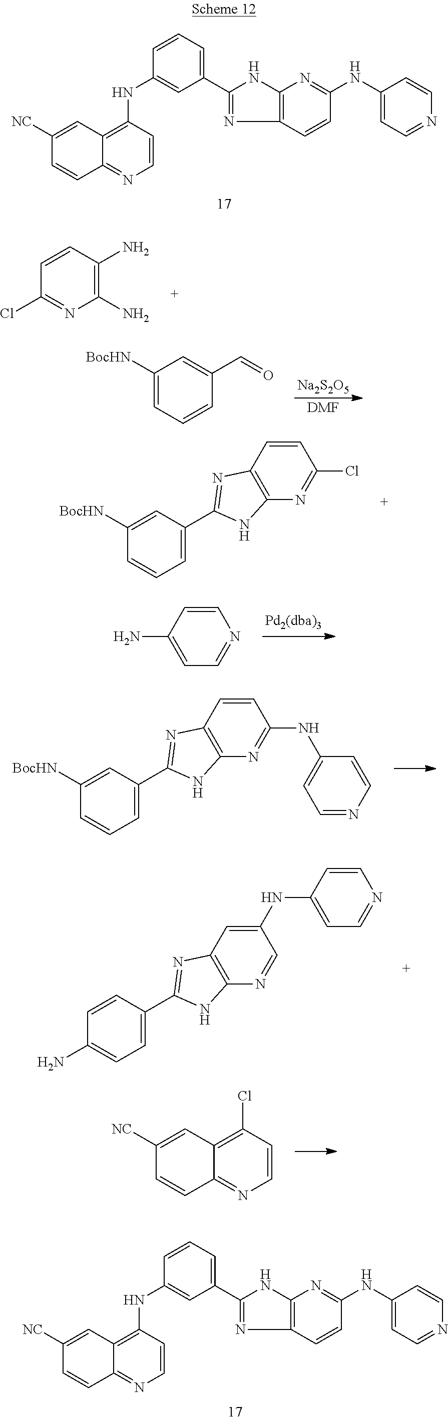

Compound 17 was prepared by the method shown in Scheme 12. Compound 17 (4-((3-(5-(pyridin-4-ylamino)-3H-imidazo[4,5-b]pyridin-2-yl)phenyl)amino)quinoline-6-carbonitrile) was prepared as shown in Scheme 12: C₂₇H₁₈N₈; 454.50 g/mol; 13 mg; yellow solid; ESI-LCMS m/z=455 [M+H]⁺; RT=1.44 min, >95.00% (214 nm).

### Example 18: Compound 18 (3-((5-methylquinolin-4-yl)amino)-N-(4-(pyridin-4-ylamino)phenyl)benzamide)

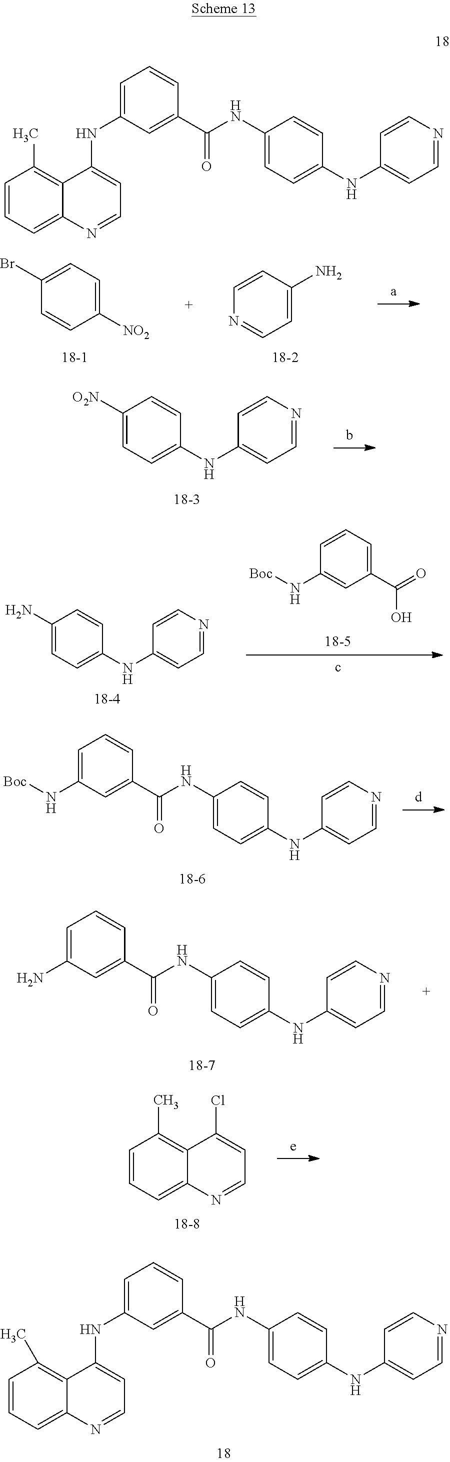

Step a: To a stirred mixture of Compound 18-1 (20 g, 0.1 mol) in 1,4-dioxane (500 mL) was added pyridin-4-amine (Compound 18-2) (9.4 g, 0.1 mol), Cs₂CO₃ (65 g, 0.2 mol), Pd₂(dba)₃ (457 mg, 0.5 mmol), and Xantphos (457 mg, 0.8 mmol) under nitrogen atmosphere. The resulting mixture was stirred at 100° C. for 2 hours. The reaction was then quenched with water (500 mL) and extracted with EA (3× 500 mL). The combined organic phase was dried over Na₂SO₄, filtered, and concentrated. The residue was purified by flash chromatography on silica gel (0-50% EA in PE) to afford Compound 18-3 (19.8 g, 93%) as a yellow solid.

Step b: To a mixture of Compound 18-3 (19.8 g, 93 mmol) in MeOH (1000 mL) was added Pd/C (986 mg, 0.93 mmol), and the mixture was stirred at room temperature for 4 hours under H₂. The combined organic phase was filtered by diatomite to give Compound 18-4 as yellow solid (16.9 g, 98.5%).

Step c: To a mixture of Compound 18-4 (16.9 g, 92 mmol) in DMF (250 mL) was added 3-((tert-butoxycarbonyl)amino)benzoic acid (Compound 18-5) (21.8 g, 92 mmol), EDCI (9.1 mg, 0.01 mmol), and DMAP (22.4 g, 184 mmol), and the mixture was stirred at room temperature for 16 hours. The reaction was then quenched with water (1000 mL) and extracted with EA (3×600 mL). The combined organic phase was dried over Na₂SO₄, filtered, and concentrated. The residue was purified by flash chromatography on silica gel (0-50% EA in PE) to afford Compound 18-6 (30.2 g, 81.2%) as a white solid.

Step d: A mixture of Compound 18-6 (30.2 g, 74 mmol) in dioxane hydrochloride (1000 mL, 4 M) was stirred at room temperature for 4 hours. The combined organic phase was concentrated to give Compound 18-7 as white solid (20.2 g, 90%).

Step e: To a mixture of Compound 18-7 (50 mg, 0.16 mmol) in DMSO (2 mL) was added 4-chloro-3-methylquinoline (Compound 18-8) (29 mg, 0.16 mmol), and a drop of hydrochloric acid. The mixture was stirred at 100° C. for 1 hour. The crude residue was purified by prep-HPLC to give Compound 18 (3-((5-methylquinolin-4-yl)amino)-N-(4-(pyridin-4-ylamino)phenyl)benzamide) as a pale yellow solid (11 mg, 15.0%): C₂₈H₂₃N₅O; 445.53 g/mol; ESI-LCMS m/z=446 [M+H]⁺; RT=1.49 min, >95.00% (214 nm).

### Example 19: Compound 19 (4-(2-aminopyridin-4-ylamino)-N-(3-(pyridin-4-ylamino)phenyl)benzamide)

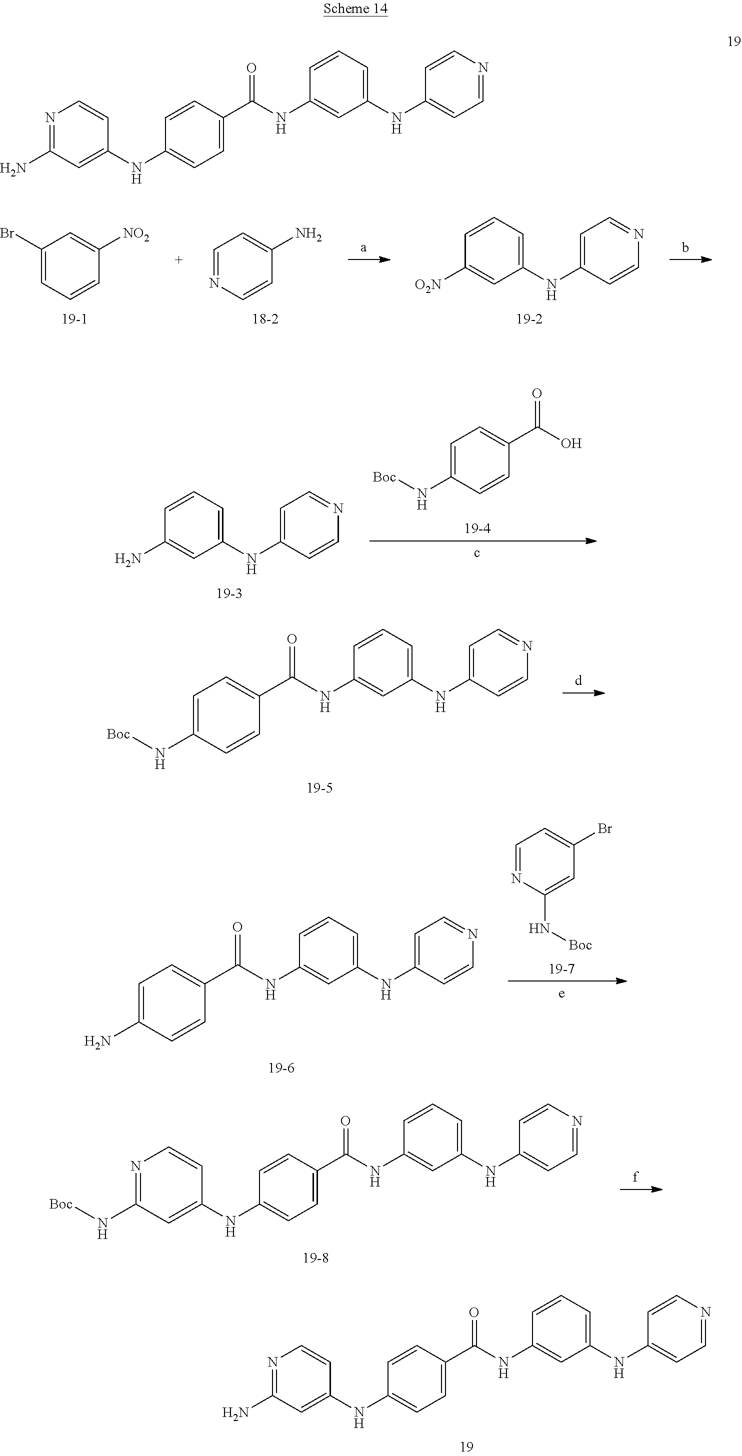

Step a: To a stirred mixture of 1-bromo-3-nitrobenzene (Compound 19-1) (20 g, 0.1 mol) in 1,4-dioxane (500 mL) was added pyridin-4-amine (Compound 18-2) (9.4 g, 0.1 mol), Cs₂CO₃ (65 g, 0.2 mol), Pd₂(dba)₃ (457 mg, 0.5 mmol), and Xantphos (457 mg, 0.8 mmol) under nitrogen atmosphere. The resulting mixture was stirred at 100° C. for 2 hours. The reaction was then quenched with water (500 mL) and extracted with EA (3× 500 mL). The combined organic phase was dried over Na₂SO₄, filtered, and concentrated. The residue was purified by flash chromatography on silica gel (0-50% EA in PE) to afford Compound 19-2 (20 g, 93%) as a yellow solid.

Step b: To a mixture of Compound 19-2 (20 g, 93 mmol) in MeOH (1000 mL) was added Pd/C (986 mg, 0.93 mmol), and the mixture was stirred at room temperature for 4 hours under H₂. The combined organic phase was filtered by diatomite to give Compound 19-3 as yellow solid (17.0 g, 98.7%).

Step c: To a mixture of Compound 19-3 (17.0 g, 92 mmol) in DMF (250 mL) was added 4-((tert-butoxycarbonyl)amino)benzoic acid (Compound 19-4) (21.8 g, 92 mmol), EDCI (9.1 mg, 0.01 mmol), and DMAP (22.4 g, 184 mmol), and the mixture was stirred at room temperature for 16 hours. The reaction was then quenched with water (1000 mL) and extracted with EA (3× 600 mL). The combined organic phase was dried over Na₂SO₄, filtered, and concentrated. The residue was purified by flash chromatography on silica gel (0-50% EA in PE) to afford Compound 19-5 (30 g, 81%) as a white solid.

Step d: A mixture of Compound 19-5 (30 g, 74 mmol) in dioxane hydrochloride (1000 mL, 4 M) was stirred at room temperature for 4 hours. The combined organic phase was concentrated to give compound 19-6 as white solid (20.2 g, 90%).

Step e: To a mixture of Compound 19-6 (50 mg, 0.164 mmol) in 1,4-dioxane (2 mL) was added tert-butyl (4-bromopyridin-2-yl) carbamate (Compound 19-7) (45 mg, 0.164 mmol), Pd₂(dba)₃ (9.1 mg, 0.01 mmol), Xantphos (6 mg, 0.01 mmol), and Cs₂CO₃ (102 mg, 0.32 mmol), and the mixture was stirred at 100° C. for 12 hours under N₂. The mixture was concentrated to give Compound 19-8, which was used directly in the next step without further purification.

Step f: A solution of Compound 19-8 in TFA (3 mL) was stirred at room temperature for 1 hour. The mixture was concentrated and the crude residue was purified by prep-HPLC to give Compound 19 (4-(2-aminopyridin-4-ylamino)-N-(3-(pyridin-4-ylamino)phenyl)benzamide) as a white solid (12 mg, 18.5%): C₂₃H₂₀N₆O; 396.44 g/mol; ESI-LCMS m/z=397 [M+H]⁺; RT=1.44 min, >95.00% (214 nm).

### Example 20: Compound 20 (4-(2-amino-6-methylpyridin-4-ylamino)-N-(3-(pyridin-4-ylamino)phenyl)benzamide)

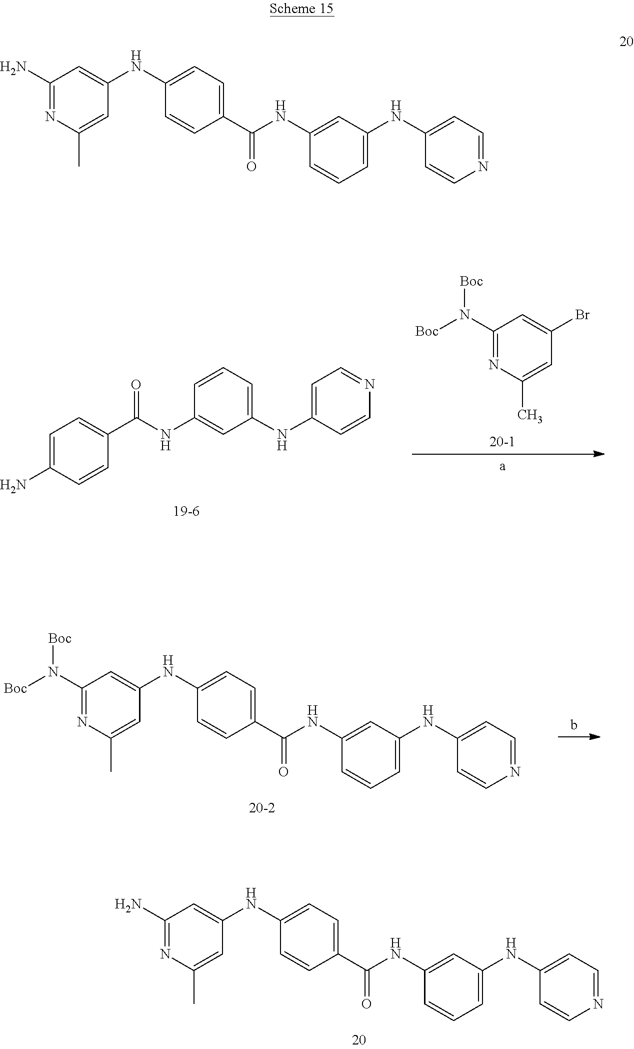 [SMILES: Cc1cc(Nc2ccc(C(=O)Nc3cccc(Nc4ccncc4)c3)cc2)cc(N)n1 ; Nc1ccc(C(=O)Nc2cccc(Nc3ccncc3)c2)cc1 ; Cc1cc(Br)cc(N(C(=O)OC(C)(C)C)C(=O)OC(C)(C)C)n1 ; Cc1cc(Nc2ccc(C(=O)Nc3cccc(Nc4ccncc4)c3)cc2)cc(N(C(=O)OC(C)(C)C)C(=O)OC(C)(C)C)n1 ; Cc1cc(Nc2ccc(C(=O)Nc3cccc(Nc4ccncc4)c3)cc2)cc(N)n1]

Compound 19-6 was prepared in an analogous fashion to the procedure described in Example 19.

Step a: To a mixture of Compound 19-6 (50 mg, 0.164 mmol) in 1,4-dioxane (2 mL) was added tert-butyl (4-bromopyridin-2-yl) carbamate (Compound 20-1) (45 mg, 0.164 mmol), Pd₂(dba)₃ (9.1 mg, 0.01 mmol), Xantphos (6 mg, 0.01 mmol), and Cs₂CO₃ (102 mg, 0.32 mmol), and the mixture was stirred at 100° C. for 12 hours under N₂. The mixture was concentrated to give Compound 20-2, which was used directly in the next step without further purification.

Step b: A solution of Compound 20-2 in TFA (3 mL) was stirred at room temperature for 1 hour. The mixture was concentrated and the crude residue was purified by prep-HPLC to give Compound 20 (4-(2-amino-6-methylpyridin-4-ylamino)-N-(3-(pyridin-4-ylamino)phenyl)benzamide) as a white solid (12 mg, 18.5%): C₂₄H₂₂N₆O; 410.47 g/mol; ESI-LCMS m/z=411 [M+H]⁺; RT=1.47 min, >95.00% (214 nm).

### Example 21: Compound 21 (4-(2-amino-3-methylpyridin-4-ylamino)-N-(3-(pyridin-4-ylamino)phenyl)benzamide)

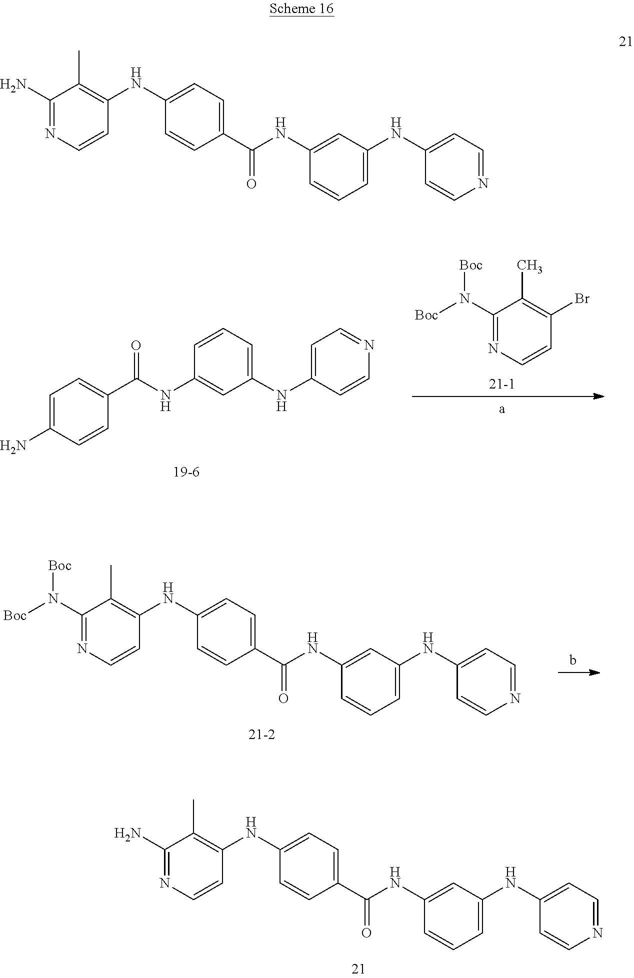 [SMILES: Cc1c(Nc2ccc(C(=O)Nc3cccc(Nc4ccncc4)c3)cc2)ccnc1N ; Nc1ccc(C(=O)Nc2cccc(Nc3ccncc3)c2)cc1 ; Cc1c(Br)ccnc1N(C(=O)OC(C)(C)C)C(=O)OC(C)(C)C ; Cc1c(Nc2ccc(C(=O)Nc3cccc(Nc4ccncc4)c3)cc2)ccnc1N(C(=O)OC(C)(C)C)C(=O)OC(C)(C)C ; Cc1c(Nc2ccc(C(=O)Nc3cccc(Nc4ccncc4)c3)cc2)ccnc1N]

Compound 19-6 was prepared in an analogous fashion to the procedure described in Example 19.

Step a: To a mixture of Compound 19-6 (50 mg, 0.164 mmol) in 1,4-dioxane (2 mL) was added Compound 21-1 (45 mg, 0.164 mmol), Pd₂(dba)₃ (9.1 mg, 0.01 mmol), Xantphos (6 mg, 0.01 mmol), and Cs₂CO₃ (102 mg, 0.32 mmol), and the mixture was stirred at 100° C. for 12 hours under N₂. The mixture was concentrated give Compound 20-2, which was used directly in the next step without further purification.

Step b: A solution of Compound 21-2 in TFA (3 mL) was stirred at room temperature for 1 hour. The mixture was concentrated and the crude residue was purified by prep-HPLC to give Compound 21 (4-(2-amino-3-methylpyridin-4-ylamino)-N-(3-(pyridin-4-ylamino)phenyl)benzamide) as a white solid (12 mg, 18.5%): C₂₄H₂₂N₆O; 410.47 g/mol; ESI-LCMS m/z=411 [M+H]⁺; RT=1.42 min, >95.00% (214 nm).

### Example 22: Compound 22 (4-((2-(4-(pyridin-4-ylamino)phenyl)-1H-benzo[d]imidazol-5-yl)amino)quinoline-6-carbonitrile)

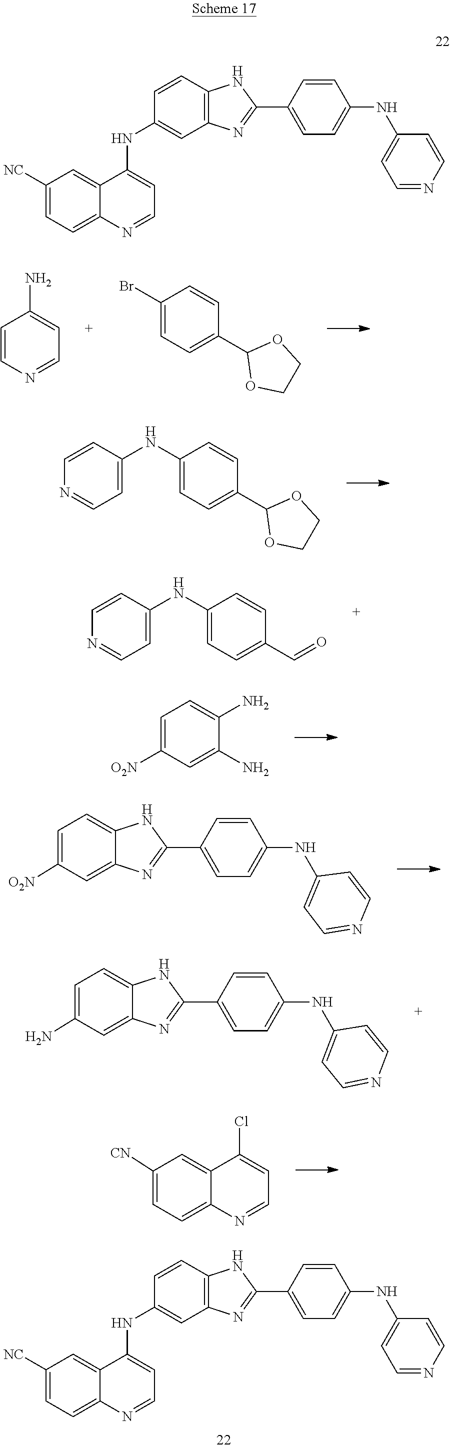

Compound 22 was prepared by the method shown in Scheme 17. Compound 22 (4-((2-(4-(pyridin-4-ylamino)phenyl)-1H-benzo[d]imidazol-5-yl)amino)quinoline-6-carbonitrile) was prepared as shown in Scheme 32: C₂₈H₁₉N₇; 453.51 g/mol; 11 mg; pale yellow solid; ESI-LCMS m/z=454 [M+H]⁺; LCMS RT=1.35 min, >95.00% (214 nm).

### Example 23: Compound 23 (3,3-dimethyl-5-(pyridin-4-ylamino)-2-(3-(pyridin-4-ylamino)phenyl)isoindolin-1-one)

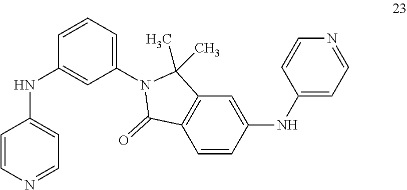 [SMILES: CC1(C)c2cc(Nc3ccncc3)ccc2C(=O)N1c1cccc(Nc2ccncc2)c1]

Compound 23 can be prepared by a method known in the art and/or a method analogous to those described herein.

### Example 24: Compound 24 (6′-(pyridin-4-ylamino)-2′-(3-(pyridin-4-ylamino)phenyl)spiro[cyclopropane-1,1′-isoindolin]-3′-one)

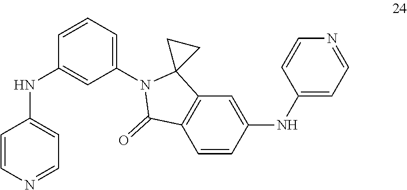 [SMILES: O=C1c2ccc(Nc3ccncc3)cc2C2(CC2)N1c1cccc(Nc2ccncc2)c1]

Compound 24 can be prepared by a method known in the art and/or a method analogous to those described herein.

### Example 25: Compound 25 (2-(2-methyl-5-(pyridin-4-ylamino)phenyl)-5-(pyridin-4-ylamino)isoindolin-1-one)

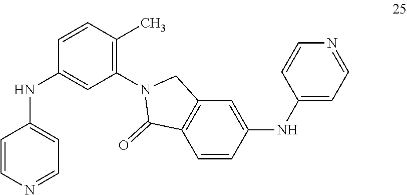 [SMILES: Cc1ccc(Nc2ccncc2)cc1N1Cc2cc(Nc3ccncc3)ccc2C1=O]

Compound 25 was prepared by a method known in the art and/or a method analogous to those described herein. Compound 25 (2-(2-methyl-5-(pyridin-4-ylamino)phenyl)-5-(pyridin-4-ylamino)isoindolin-1-one): C₂₅H₂₁N₅O; 407.47 g/mol; 14 mg; off-white solid; ESI-LCMS m/z=408 [M+H]⁺; LCMS RT=1.48 min, >95.00% (214 nm).

### Example 26: Compound 26 (2-(3-methyl-5-(pyridin-4-ylamino)phenyl)-5-(pyridin-4-ylamino)isoindolin-1-one)

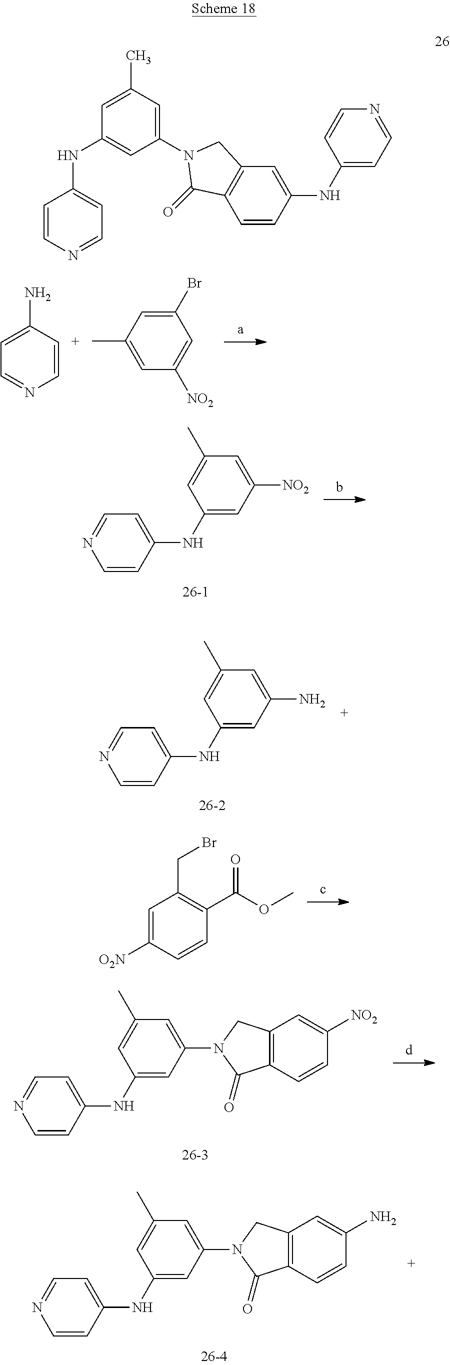

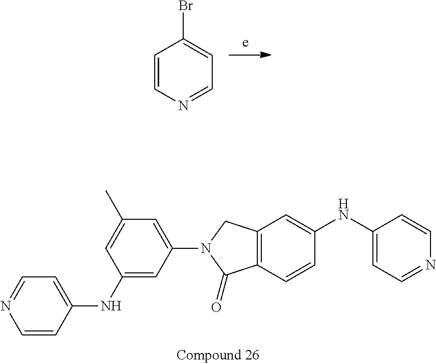 [SMILES: Brc1ccncc1 ; Cc1cc(Nc2ccncc2)cc(N2Cc3cc(Nc4ccncc4)ccc3C2=O)c1]

Compound 26 was prepared by the method shown in Scheme 18.

Step a: To a stirred mixture of 1-bromo-3-methyl-5-nitrobenzene (2.15 g, 0.01 mol) in 1,4-dioxane (50 mL) was added pyridin-4-amine (0.94 g, 0.01 mol), Cs₂CO₃ (6.5 g, 0.02 mol), Pd₂(dba)₃ (45.7 mg, 0.05 mmol), and Xantphos (45.7 mg, 0.08 mmol) under N₂. The resulting mixture was stirred at 100° C. for 4 hours. The reaction was then quenched with water (50 mL) and extracted with EtOAc (3×50 mL). The combined organic phases were dried over Na₂SO₄, filtered, and concentrated. The residue was purified by flash chromatography on silica gel (0-50% EtOAc in petroleum ether) to afford Compound 26-1 (2.0 g, 90% yield) as a yellow solid.

Step b: To a mixture of Compound 26-1 (2.0 g, 8.7 mmol) in MeOH (50 mL) was added Pd/C (200 mg, 0.87 mmol), and the mixture was stirred at room temperature for 4 hours under H₂. The combined organic phases were filtered to give Compound 26-2 as yellow solid (1.5 g, 86.7% yield).

Step c: To a mixture of Compound 26-2 (1.5 g, 7.5 mmol) and methyl 2-(bromomethyl)-4-nitrobenzoate (2.05 g, 7.5 mmol) in MeOH (50 mL) was added TEA (2.28 g, 22.6 mmol), and the mixture was stirred at 85° C. for 16 hours. The combined organic phases were concentrated. The residue was purified by flash chromatography on silica gel (0-80% EtOAc in petroleum ether) to afford Compound 26-3 (500 mg, 18.5% yield) as a yellow solid.

Step d: To a mixture of Compound 26-3 (300 mg, 0.83 mmol) in MeOH (10 mL)/THF (5 mL) was added Pd/C (50 mg), and the mixture was stirred at 60° C. for 5 hours under H₂. The combined organic phases were filtered by diatomite to give Compound 26-4 (150 mg, 54% yield) as a yellow solid.

Step e: To a mixture of Compound 26-4 (150 mg, 0.45 mmol) in 1,4-dioxane (6 mL) was added 4-bromopyridine (72 mg, 0.45 mmol), Pd₂(dba)₃ (18 mg, 0.02 mmol), Xantphos (12 mg, 0.02 mmol), and Cs₂CO₃ (293 mg, 0.9 mmol), and the mixture was stirred at 100° C. for 12 hours under N₂. The mixture was concentrated and the crude was purified by prep-HPLC to give Compound 26 as a yellow solid. (20 mg, 10.8% yield).

Compound 26 ((2-(3-methyl-5-(pyridin-4-ylamino)phenyl)-5-(pyridin-4-ylamino)isoindolin-1-one): C₂₅H₂₁N₅O; 407.47 g/mol; 20 mg; white solid; ESI-LCMS m/z=408 [M+H]⁺; LCMS RT=1.67 min, >95.00% (214 nm).

### Example 27: Compound 27 (2-(4-methyl-3-(pyridin-4-ylamino)phenyl)-5-(pyridin-4-ylamino)isoindolin-1-one)

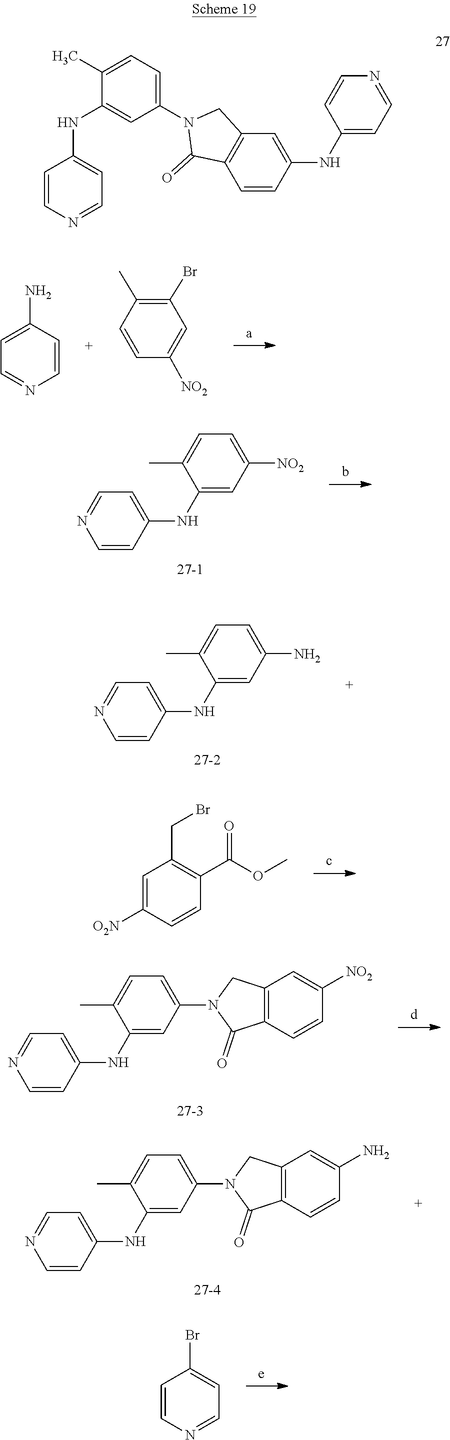

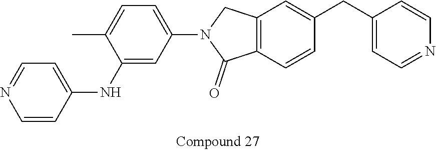 [SMILES: Cc1ccc(N2Cc3cc(Cc4ccncc4)ccc3C2=O)cc1Nc1ccncc1]

Compound 27 was prepared by the method shown in Scheme 19.

Step a: To a stirred mixture of 2-bromo-1-methyl-4-nitrobenzene (2.15 g, 0.01 mol) in 1,4-dioxane (50 mL) was added pyridin-4-amine (0.94 g, 0.01 mol), Cs₂CO₃ (6.5 g, 0.02 mol), Pd₂(dba)₃ (45.7 mg, 0.05 mmol), and Xantphos (45.7 mg, 0.08 mmol) under N₂. The resulting mixture was stirred at 100° C. for 4 hours. The reaction was quenched with water (50 mL) and extracted with EtOAc (3×50 mL). The combined organic phases were dried over Na₂SO₄, filtered, and concentrated. The residue was purified by flash chromatography on silica gel (0-50% EtOAc in petroleum ether) to afford Compound 27-1 (1.9 g, 83% yield) as a yellow solid.

Step b: To a mixture of Compound 27-1 (1.9 g, 8.3 mmol) in MeOH (30 mL) was added Pd/C (200 mg, 0.87 mmol), and the mixture was stirred at room temperature for 5 hours under H₂. The combined organic phases were filtered to give Compound 27-2 as a yellow solid (1.5 g, 90.9% yield).

Step c: To a mixture of Compound 27-2 (1.5 g, 7.5 mmol) and methyl 2-(bromomethyl)-4-nitrobenzoate (2.05 g, 7.5 mmol) in MeOH (50 mL) was added TEA (2.28 g, 22.6 mmol), and the mixture was stirred at 85° C. for 16 hours. The combined organic phases were concentrated. The residue was purified by flash chromatography on silica gel (0-20% MeOH in DCM) to afford Compound 27-3 (700 mg, 25.9% yield) as a yellow solid.

Step d: To a mixture of Compound 27-3 (250 mg, 0.83 mmol) in MeOH (10 mL)/THF (5 mL) was added Pd/C (50 mg), and the mixture was stirred at 60° C. for 5 hours under H₂. The combined organic phases were filtered to give Compound 27-4 as a yellow solid (170 mg, 74% yield).

Step e: To a mixture of Compound 27-4 (100 mg, 0.30 mmol) in 1,4-dioxane (6 mL) was added 4-bromopyridine (47 mg, 0.3 mmol), Pd₂(dba)₃ (18 mg, 0.02 mmol), Xantphos (12 mg, 0.02 mmol), and Cs₂CO₃ (195 mg, 0.6 mmol), and the mixture was stirred at 100° C. for 12 hours under N₂. The mixture was concentrated and the crude was purified by prep-HPLC to give Compound 27 as a yellow solid. (15 mg, 12.2% yield).

Compound 27 (2-(4-methyl-3-(pyridin-4-ylamino)phenyl)-5-(pyridin-4-ylamino)isoindolin-1-one): C₂₅H₂₁NO; 407.48 g/mol; 15 mg; white solid; ESI-LCMS m/z=408 [M+H]⁺; LCMS RT=1.64 min, >95.00% (214 nm).

### Example 28: Compound 28 (2-(2-methyl-3-(pyridin-4-ylamino)phenyl)-5-(pyridin-4-ylamino)isoindolin-1-one)

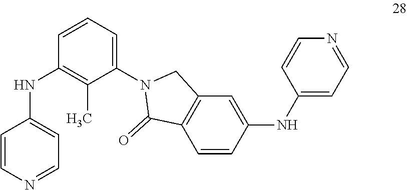 [SMILES: Cc1c(Nc2ccncc2)cccc1N1Cc2cc(Nc3ccncc3)ccc2C1=O]

Compound 28 was prepared by a method known in the art and/or a method analogous to those described herein. Compound 28 (2-(2-methyl-3-(pyridin-4-ylamino)phenyl)-5-(pyridin-4-ylamino)isoindolin-1-one): C₂₅H₂₁N₅O; 407.47 g/mol; 13 mg; white solid; ESI-LCMS m/z=408 [M+H]⁺; LCMS RT=1.44 min, >95.00% (214 nm).

### Example 29: Compound 29 (4-methyl-5-(pyridin-4-ylamino)-2-(3-(pyridin-4-ylamino)phenyl)isoindolin-1-one)

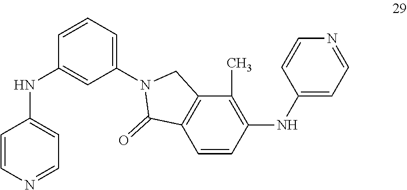 [SMILES: Cc1c(Nc2ccncc2)ccc2c1CN(c1cccc(Nc3ccncc3)c1)C2=O]

Compound 29 was prepared by a method known in the art and/or a method analogous to those described herein. Compound 29 (4-methyl-5-(pyridin-4-ylamino)-2-(3-(pyridin-4-ylamino)phenyl)isoindolin-1-one): C₂₅H₂₁N₅O; 407.47 g/mol; 10 mg; white solid; ESI-LCMS m/z=408 [M+H]⁺; LCMS RT=1.34 min, >95.00% (214 nm).

### Example 30: Compound 30 (7-methyl-5-(pyridin-4-ylamino)-2-(3-(pyridin-4-ylamino)phenyl)isoindolin-1-one)

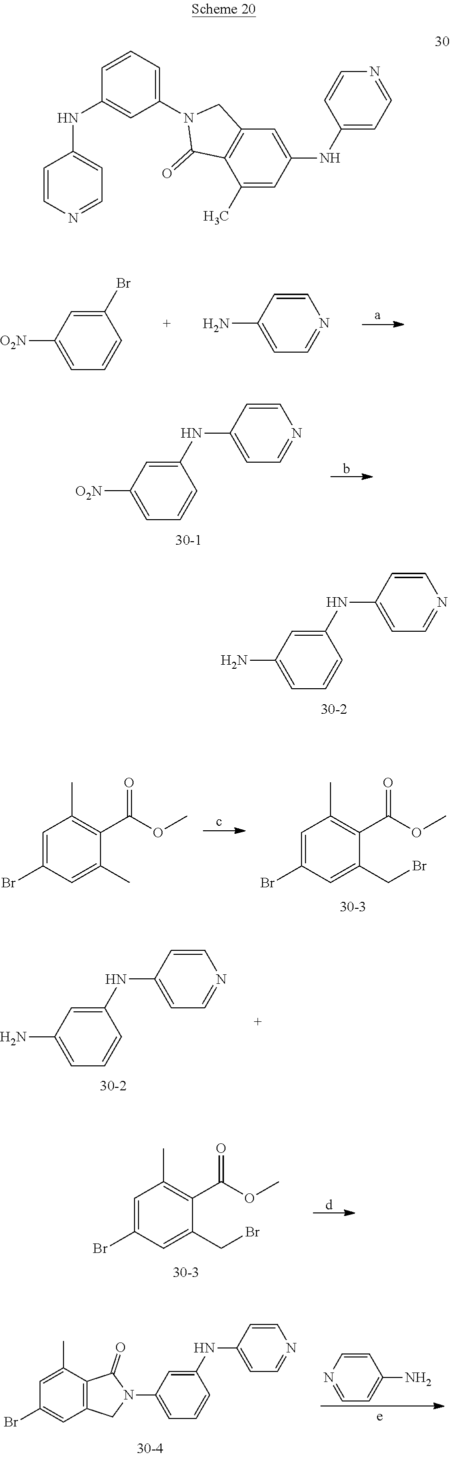

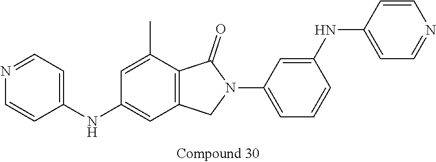 [SMILES: Cc1cc(Nc2ccncc2)cc2c1C(=O)N(c1cccc(Nc3ccncc3)c1)C2]

Compound 30 was prepared by the method shown in Scheme 20.

Step a: To a stirred mixture of 1-bromo-3-nitrobenzene (10 g, 0.05 mol) in 1,4-dioxane (200 mL) was added pyridin-4-amine (4.7 g, 0.05 mol), Cs₂CO₃ (32.5 g, 0.1 mol), Pd₂(dba)₃ (230 mg, 0.25 mmol), and Xantphos (230 mg, 0.4 mmol) under N₂. The resulting mixture was stirred at 100° C. for 3 hours. The reaction was quenched with water (300 mL) and extracted with EtOAc (3×100 mL). The combined organic phases were dried over Na₂SO₄, filtered, and concentrated. The residue was purified by flash chromatography on silica gel (0-50% EtOAc in petroleum ether) to afford Compound 30-1 (10 g, 93.4% yield) as a yellow solid.

Step b: To a mixture of Compound 30-1 (10 g, 46.5 mmol) in MeOH (300 mL) was added Pd/C (1 g), and the mixture was stirred at room temperature for 4 hours under H₂. The combined organic phases were filtered by diatomite to give Compound 30-2 as yellow solid (8.5 g, 98.7% yield).

Step c: To a mixture of methyl 4-bromo-2,6-dimethylbenzoate (1.21 g, 5 mmol) and NBS (0.979 g, 5.5 mmol) in CCl₄ (15 mL) was added AIBN (150 mg), and the mixture was stirred at 80° C. for 16 hours. The combined organic phases were concentrated. The residue was purified by flash chromatography on silica gel (0-20% DCM in petroleum ether) to give Compound 30-3 (1.2 g, 75% yield) as a solid.

Step d: To a mixture of Compound 30-3 (640 mg, 2 mmol) and 30-2 (407 mg, 2.2 mmol) in MeOH (10 mL) was added TEA (606 mg, 6 mmol), and the mixture was stirred at 85° C. for 16 hours. The combined organic phases were concentrated. The residue was purified by flash chromatography on silica gel (0-20% MeOH in DCM) to afford Compound 30-4 (400 mg, 50.8% yield) as a solid.

Step e: To a mixture of Compound 30-4 (150 mg, 0.38 mmol) in 1,4-dioxane (6 mL) was added pyridin-4-amine (36 mg, 0.38 mmol), Pd₂(dba)₃ (18 mg, 0.02 mmol), Xantphos (12 mg, 0.02 mmol), and Cs₂CO₃ (234 mg, 0.72 mmol), and the mixture was stirred at 100° C. for 12 hours under N₂. The mixture was concentrated and the crude was purified by prep-HPLC to give Compound 30 as a yellow solid. (18 mg, 11.6% yield).

Compound 30 (2-(7-methyl-5-(pyridin-4-ylamino)-2-(3-(pyridin-4-ylamino)phenyl)isoindolin-1-one): C₂₅H₂₁N₅O; 407.47 g/mol; 18 mg; white solid; ESI-LCMS m/z=408 [M+H]⁺; LCMS RT=1.37 min, >95.00% (214 nm).

### Example 31: Compound 31 (6-methyl-5-(pyridin-4-ylamino)-2-(3-(pyridin-4-ylamino)phenyl)isoindolin-1-one)

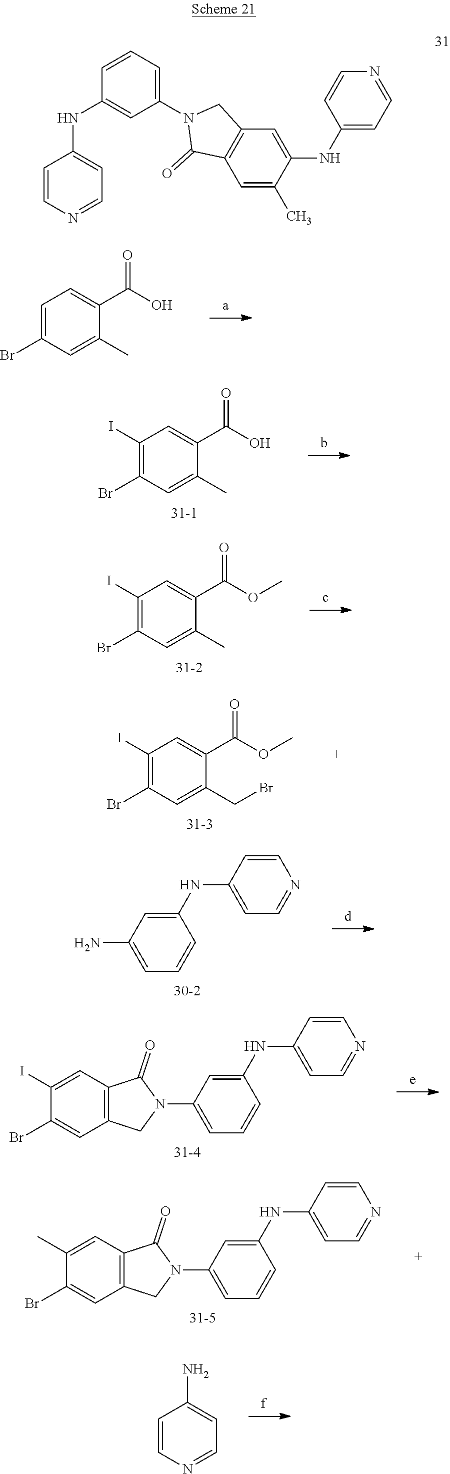

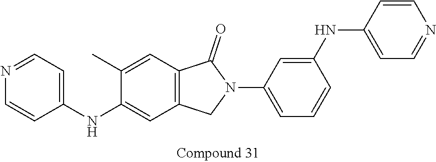 [SMILES: Cc1cc2c(cc1Nc1ccncc1)CN(c1cccc(Nc3ccncc3)c1)C2=O]

Compound 31 was prepared by the method shown in Scheme 21.

Step a: To a mixture of 4-bromo-2-methylbenzoic acid (2.14 g, 10 mmol) in concentrated H₂SO₄ (20 mL) at 0° C. was added NIS (2.47 g, 11 mmol), and the mixture was stirred at 0° C. for 3 hours. The mixture was poured onto ice and filtered to give Compound 31-1 as a yellow solid (2.5 g, 73% yield).

Step b: To a mixture of Compound 31-1 (1.7 g, 5 mmol) and Cs₂CO₃ (234 mg, 10 mmol) in DMF (10 mL) was added MeI (965 mg, 6.8 mmol), and the mixture was stirred at room temperature for 1 hour. The reaction was quenched with water (50 mL) and extracted with EtOAc (3×20 mL). The combined organic phases were dried over Na₂SO₄, filtered, and concentrated. The residue was purified by flash chromatography on silica gel (0-30% EtOAc in petroleum ether) to afford Compound 31-2 (1.6 g, 90% yield).

Step c: To a mixture of Compound 31-2 (1.5 g, 4.2 mmol) and NBS (830 mg, 4.66 mmol) in CCl₄ (20 mL) was added AIBN (150 mg), and the mixture was stirred at 80° C. for 16 hours. The combined organic phases were concentrated. The residue was purified by flash chromatography on silica gel (0-20% DCM in petroleum ether) to afford Compound 31-3 (1.46 g, 80% yield) as a solid.

Step d: To a mixture of Compound 31-3 (1.3 g, 3 mmol) and Compound 30-2 (610 mg, 3.3 mmol) in MeOH (20 mL) was added TEA (909 mg, 9 mmol), and the mixture stirred at 85° C. for 16 hours. The combined organic phases were concentrated. The residue was purified by flash chromatography on silica gel (0-20% MeOH in DCM) to afford Compound 31-4 (1.1 g, 79.8% yield) as a solid.

Step e: To a stirred mixture of Compound 31-4 (1.1 g, 2 mmol) in 1,4-dioxane (30 mL) was added 2,4,6-trimethyl-1,3,5,2,4,6-trioxatriborinane (151.2 mg, 1.2 mol), K₂CO₃ (552 mg, 4 mmol), and Pd(dppf)Cl₂ (230 mg, 0.25 mmol) under N₂. The resulting mixture was stirred at 100° C. for 16 hours then filtered and concentrated. The residue was purified by flash chromatography on silica gel (0-30% MeOH in DCM) to afford Compound 31-5 (260 mg, 33% yield).

Step f: To a mixture of Compound 31-5 (250 mg, 0.63 mmol) in 1,4-dioxane (8 mL) was added pyridin-4-amine (60 mg, 1.26 mmol), Pd₂(dba)₃ (25 mg, 0.02 mmol), Xantphos (25 mg, 0.02 mmol), and Cs₂CO₃ (410 mg, 0.72 mmol), and the mixture was stirred at 100° C. for 12 hours under N₂. The mixture was concentrated and the crude was purified by prep-HPLC to give Compound 31 as a yellow solid. (25 mg, 10% yield).

Compound 31 (6-methyl-5-(pyridin-4-ylamino)-2-(3-(pyridin-4-ylamino)phenyl)isoindolin-1-one): C₂₅H₂₁N₅O; 407.47 g/mol; 25 mg; white solid; ESI-LCMS m/z=408 [M+H]⁺; LCMS RT=1.48 min, >95.00% (214 nm).

### Example 32: Compound 32 (4-fluoro-5-(pyridin-4-ylamino)-2-(3-(pyridin-4-ylamino)phenyl)isoindolin-1-one)

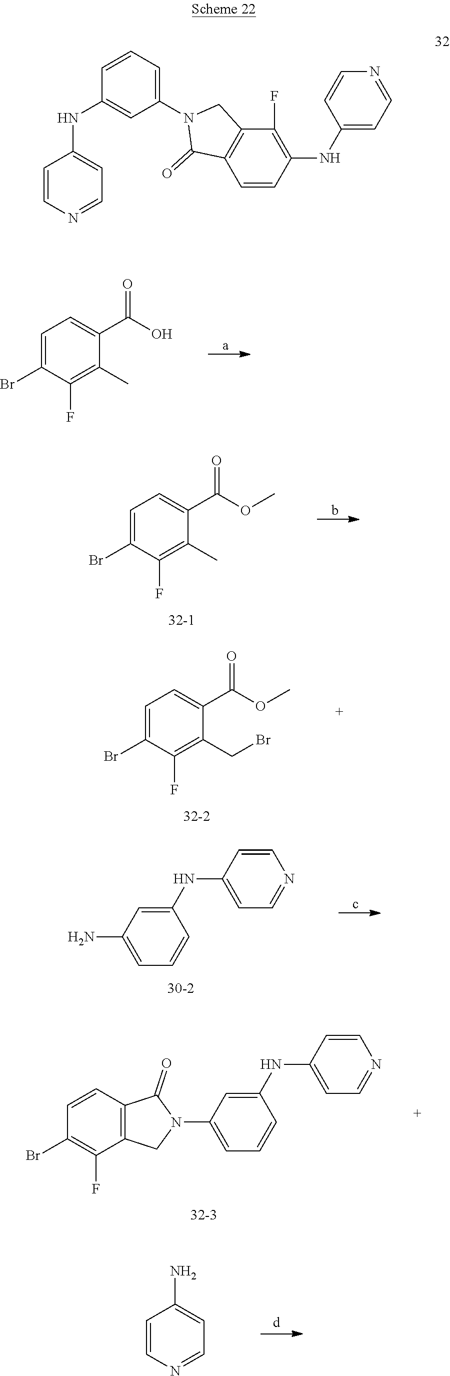

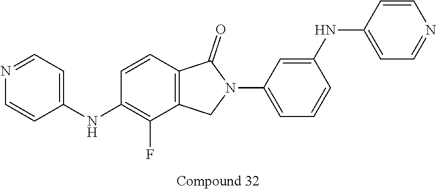 [SMILES: O=C1c2ccc(Nc3ccncc3)c(F)c2CN1c1cccc(Nc2ccncc2)c1]

Compound 32 was prepared by the method shown in Scheme 22.

Step a: To a mixture of 4-bromo-3-fluoro-2-methylbenzoic acid (500 mg, 2.15 mmol) and Cs₂CO₃ (594 mg, 4.3 mmol) in DMF (10 mL) was added MeI (368 mg, 2.58 mmol), and the mixture was stirred at room temperature for 1 hour. The reaction was quenched with water (30 mL) and extracted with EtOAc (3×20 mL). The combined organic phases were dried over Na₂SO₄, filtered, and concentrated. The residue was purified by flash chromatography on silica gel (0-30% EtOAc in petroleum ether) to afford Compound 32-1 (500 mg, 94% yield).

Step b: To a mixture of Compound 32-1 (492 g, 2 mmol) and NBS (392 mg, 2.2 mmol) in CCl₄ (10 mL) was added AIBN (50 mg), and the mixture was stirred at 80° C. for 16 hours. The combined organic phases were concentrated. The residue was purified by flash chromatography on silica gel (0-20% DCM in petroleum ether) to afford Compound 32-2 (583 mg, 90% yield) as a solid.

Step c: To a mixture of Compound 32-2 (500 mg, 1.54 mmol) and 30-2 (314 mg, 1.69 mmol) in MeOH (10 mL) was added TEA (467 mg, 4.62 mmol), and the mixture was stirred at 85° C. for 16 hours. The combined organic phases were concentrated. The residue was purified by flash chromatography on silica gel (0-20% MeOH in DCM) to afford Compound 32-3 (300 mg, 49% yield) as a solid.

Step d: To a mixture of Compound 32-3 (200 mg, 0.50 mmol) in 1,4-dioxane (8 mL) was added pyridin-4-amine (47 mg, 0.5 mmol), Pd₂(dba)₃ (20 mg, 0.02 mmol), Xantphos (20 mg, 0.02 mmol), and Cs₂CO₃ (325 mg, 0.1 mmol), and the mixture was stirred at 100° C. for 12 hours under N₂. The mixture was concentrated and the crude was purified by prep-HPLC to give Compound 32 as a yellow solid. (25 mg, 12% yield).

Compound 32 (4-fluoro-5-(pyridin-4-ylamino)-2-(3-(pyridin-4-ylamino)phenyl)isoindolin-1-one): C₂₄H₁₈FN₅O; 411.43 g/mol; 25 mg; off-white solid; ESI-LCMS m/z=412 [M+H]⁺; LCMS RT=1.35 min, >95.00% (214 nm).

### Example 33: Compound 33 (6-fluoro-5-(pyridin-4-ylamino)-2-(3-(pyridin-4-ylamino)phenyl)isoindolin-1-one)

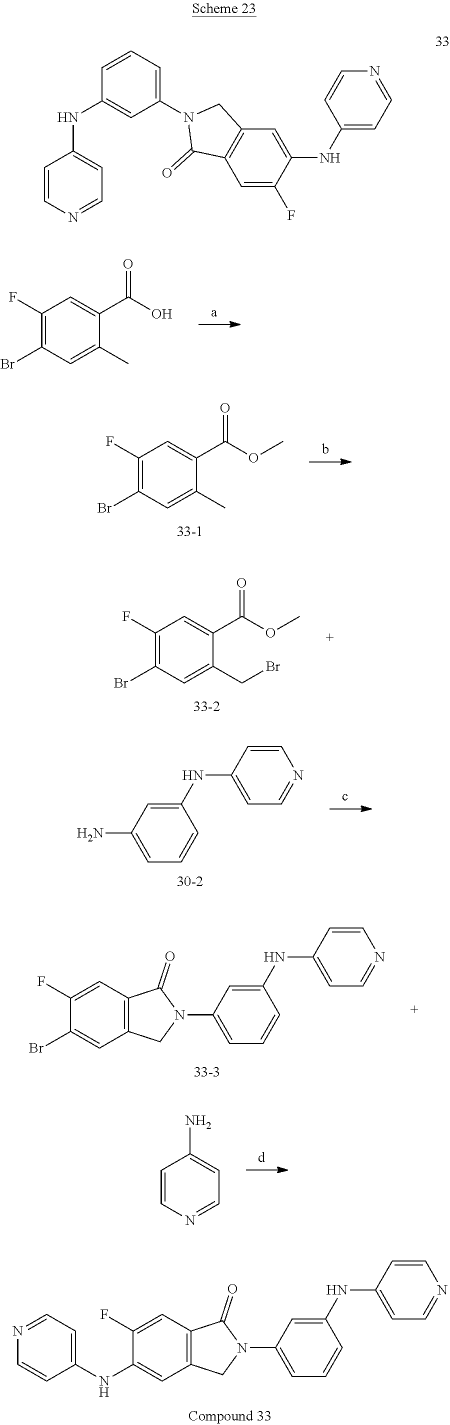

Compound 33 was prepared by the method shown in Scheme 23.

Step a: To a mixture of 4-bromo-5-fluoro-2-methylbenzoic acid (500 mg, 2.15 mmol) and Cs₂CO₃ (594 mg, 4.3 mmol) in DMF (10 mL) was added MeI (368 mg, 2.58 mmol), and the mixture was stirred at room temperature for 1 hour. The reaction was quenched with water (30 mL) and extracted with EtOAc (3× 20 mL). The combined organic phases were dried over Na₂SO₄, filtered, and concentrated. The residue was purified by flash chromatography on silica gel (0-30% EtOAc in petroleum ether) to afford Compound 33-1 (510 mg, 96% yield).

Step b: To a mixture of Compound 33-1 (500 mg, 2.03 mmol) and NBS (397 mg, 2.23 mmol) in CCl₄ (10 mL) was added AIBN (30 mg), and the mixture was stirred at 80° C. for 16 hours. The combined organic phases were concentrated. The residue was purified by flash chromatography on silica gel (0-20% DCM in petroleum ether) to afford Compound 33-2 (450 mg, 68% yield) as a solid.

Step c: To a mixture of Compound 33-2 (450 mg, 1.39 mmol) and 30-2 (283 mg, 1.52 mmol) in MeOH (10 mL) was added TEA (418 mg, 4.14 mmol), and the mixture was stirred at 85° C. for 16 hours. The combined organic phases were concentrated. The residue was purified by flash chromatography on silica gel (0-20% MeOH in DCM) to afford Compound 33-3 (300 mg, 54% yield) as a solid.

Step d: To a mixture of Compound 33-3 (200 mg, 0.50 mmol) in 1,4-dioxane (8 mL) was added pyridin-4-amine (47 mg, 0.5 mmol), Pd₂(dba)₃ (20 mg, 0.02 mmol), Xantphos (20 mg, 0.02 mmol), and Cs₂CO₃ (325 mg, 0.1 mmol), and the mixture was stirred at 100° C. for 12 hours under N₂. The mixture was concentrated and the crude was purified by prep-HPLC to give Compound 33 as a yellow solid. (20 mg, 10% yield).

Compound 33 (6-fluoro-5-(pyridin-4-ylamino)-2-(3-(pyridin-4-ylamino)phenyl)isoindolin-1-one): C₂₄H₁₈FN₅O; 411.43 g/mol; 20 mg; white solid; ESI-LCMS m/z=412 [M+H]⁺; LCMS RT=1.36 min, >95.00% (214 nm).

### Example 34: Compound 34 (7-fluoro-5-(pyridin-4-ylamino)-2-(3-(pyridin-4-ylamino)phenyl)isoindolin-1-one)

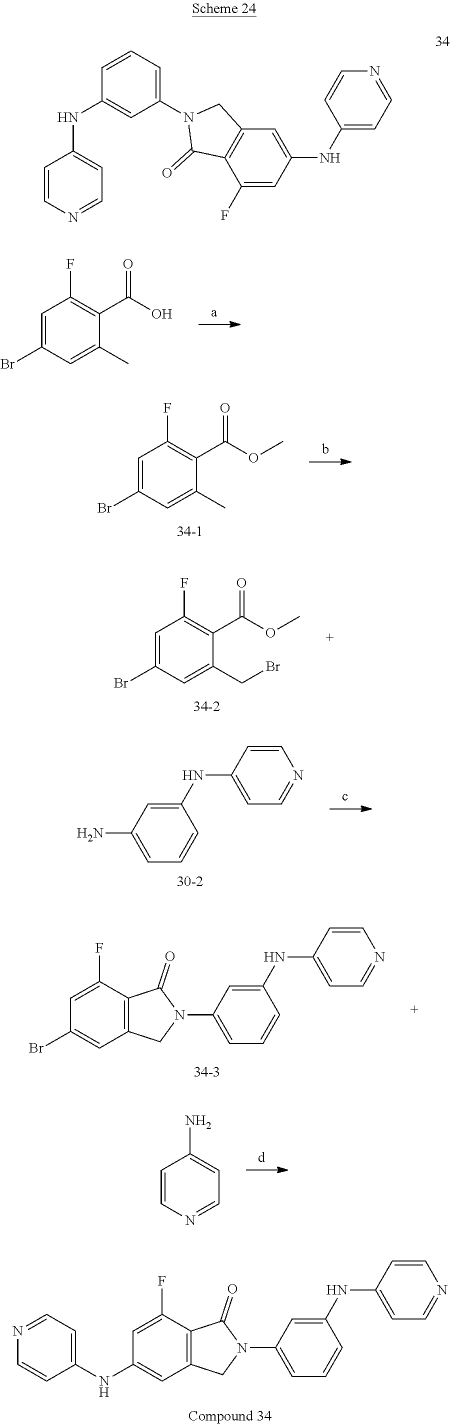

Compound 34 was prepared by the method shown in Scheme 24.

Step a: To a mixture of 4-bromo-5-fluoro-2-methylbenzoic acid (500 mg, 2.15 mmol) and Cs₂CO₃ (594 mg, 4.3 mmol) in DMF (10 mL) was added MeI (368 mg, 2.58 mmol), and the mixture was stirred at room temperature for 1 hour. The reaction was quenched with water (30 mL) and extracted with EtOAc (3×20 mL). The combined organic phases were dried over Na₂SO₄, filtered, and concentrated. The residue was purified by flash chromatography on silica gel (0-30% EtOAc in petroleum ether) to afford Compound 34-1 (500 mg, 94% yield).

Step b: To a mixture of Compound 34-1 (500 mg, 2.03 mmol) and NBS (398 mg, 2.23 mmol) in CCl₄ (10 mL) was added AIBN (30 mg), and the mixture was stirred at 80° C. for 16 hours. The combined organic phases were concentrated. The residue was purified by flash chromatography on silica gel (0-20% DCM in petroleum ether) to afford Compound 34-2 (500 mg, 76% yield) as a solid.

Step c: To a mixture of Compound 34-2 (500 mg, 1.54 mmol) and 30-2 (313 mg, 1.69 mmol) in MeOH (10 mL) was added TEA (466 mg, 4.62 mmol), and the mixture was stirred at 85° C. for 16 hours. The combined organic phases were concentrated. The residue was purified by flash chromatography on silica gel (0-20% MeOH in DCM) to afford Compound 34-3 (250 mg, 41% yield) as a solid.

Step d: To a mixture of Compound 34-3 (200 mg, 0.50 mmol) in 1,4-dioxane (8 mL) was added pyridin-4-amine (47 mg, 0.5 mmol), Pd₂(dba)₃ (20 mg, 0.02 mmol), Xantphos (20 mg, 0.02 mmol), and Cs₂CO₃ (325 mg, 0.1 mmol), and the mixture was stirred at 100° C. for 12 hours under N₂. The mixture was concentrated and the crude was purified by prep-HPLC to give Compound 34 as a yellow solid. (21 mg, 10% yield).

Compound 34 (7-fluoro-5-(pyridin-4-ylamino)-2-(3-(pyridin-4-ylamino)phenyl)isoindolin-1-one): C₂₄H₁₈FN₅O; 411.43 g/mol; 21 mg; off-white solid; ESI-LCMS m/z=412 [M+H]⁺; LCMS RT=1.26 min, >95.00% (214 nm).

### Example 35: Compound 35 (2-(2-fluoro-3-(pyridin-4-ylamino)phenyl)-5-(pyridin-4-ylamino)isoindolin-1-one)

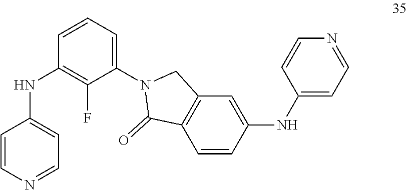 [SMILES: O=C1c2ccc(Nc3ccncc3)cc2CN1c1cccc(Nc2ccncc2)c1F]

Compound 35 was prepared by a method known in the art and/or a method analogous to those described herein. Compound 35 (2-(2-fluoro-3-(pyridin-4-ylamino)phenyl)-5-(pyridin-4-ylamino)isoindolin-1-one): C₂₄H₁₈FN₅O; 411.43 g/mol; 11 mg; off-white solid; ESI-LCMS m/z=412 [M+H]⁺; LCMS RT=1.29 min, >95.00% (214 nm).

### Example 36: Compound 36 (2-(2-fluoro-5-(pyridin-4-ylamino)phenyl)-5-(pyridin-4-ylamino)isoindolin-1-one)

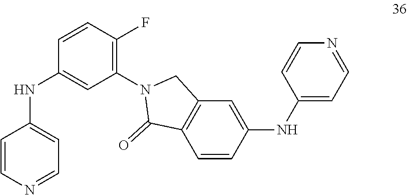 [SMILES: O=C1c2ccc(Nc3ccncc3)cc2CN1c1cc(Nc2ccncc2)ccc1F]

Compound 36 was prepared by a method known in the art and/or a method analogous to those described herein. Compound 36 (2-(2-fluoro-5-(pyridin-4-ylamino)phenyl)-5-(pyridin-4-ylamino)isoindolin-1-one): C₂₄H₁₈FN₅O; 411.43 g/mol; 11 mg; white solid; ESI-LCMS m/z=412 [M+H]⁺; LCMS RT=1.27 min, >95.00% (214 nm).

### Example 37: Compound 37 (2-(3-fluoro-5-(pyridin-4-ylamino)phenyl)-5-(pyridin-4-ylamino)isoindolin-1-one)

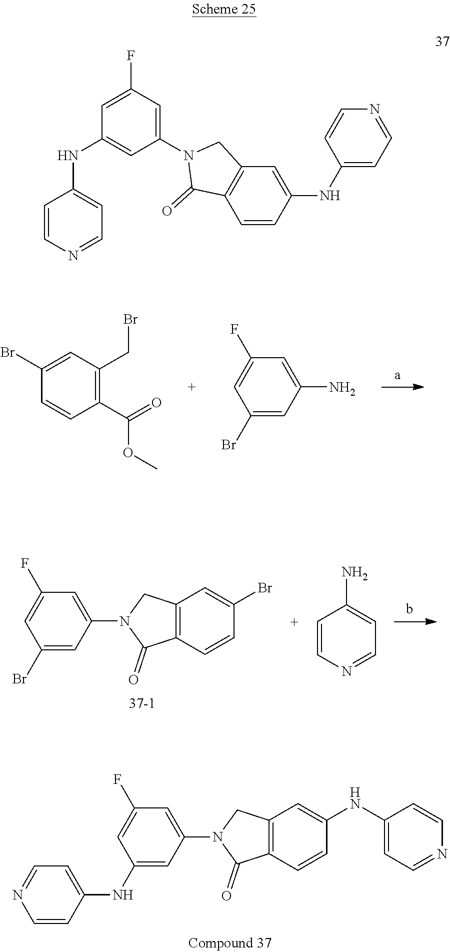 [SMILES: O=C1c2ccc(Nc3ccncc3)cc2CN1c1cc(F)cc(Nc2ccncc2)c1 ; COC(=O)c1ccc(Br)cc1CBr ; Nc1cc(F)cc(Br)c1 ; O=C1c2ccc(Br)cc2CN1c1cc(F)cc(Br)c1 ; Nc1ccncc1 ; O=C1c2ccc(Nc3ccncc3)cc2CN1c1cc(F)cc(Nc2ccncc2)c1]

Compound 37 was prepared by the method shown in Scheme 25.

Step a: To methyl 4-bromo-2-(bromomethyl)benzoate (459 mg, 1.5 mmol) was added 3-bromo-5-fluoroaniline (283 mg, 1.5 mmol), and the mixture was stirred at 135° C. for 8 hours. The reaction was quenched with MeOH (5 mL) and filtered to give Compound 37-1 as a white solid (320 mg, 55.7% yield).

Step b: To a mixture of Compound 37-1 (300 mg, 0.78 mmol) in t-BuOH (5 mL)/toluene (5 mL) was added pyridin-4-amine (220 mg, 2.34 mmol), Pd₂(dba)₃ (30 mg, 0.02 mmol), Brettphos (30 mg, 0.02 mmol), and Cs₂CO₃ (760 mg, 2.34 mmol), and the mixture stirred at 100° C. for 12 hours under N₂. The mixture was concentrated and the crude was purified by prep-HPLC to give Compound 37 as a yellow solid. (20 mg, 9% yield).

Compound 37 (2-(3-fluoro-5-(pyridin-4-ylamino)phenyl)-5-(pyridin-4-ylamino)isoindolin-1-one): C₂₄H₁₈FN₅O; 411.43 g/mol; 20 mg; off-white solid; ESI-LCMS m/z=412 [M+H]⁺; LCMS RT=1.32 min, >95.00% (214 nm).

### Example 38: Compound 38 (2-(4-fluoro-3-(pyridin-4-ylamino)phenyl)-5-(pyridin-4-ylamino)isoindolin-1-one)

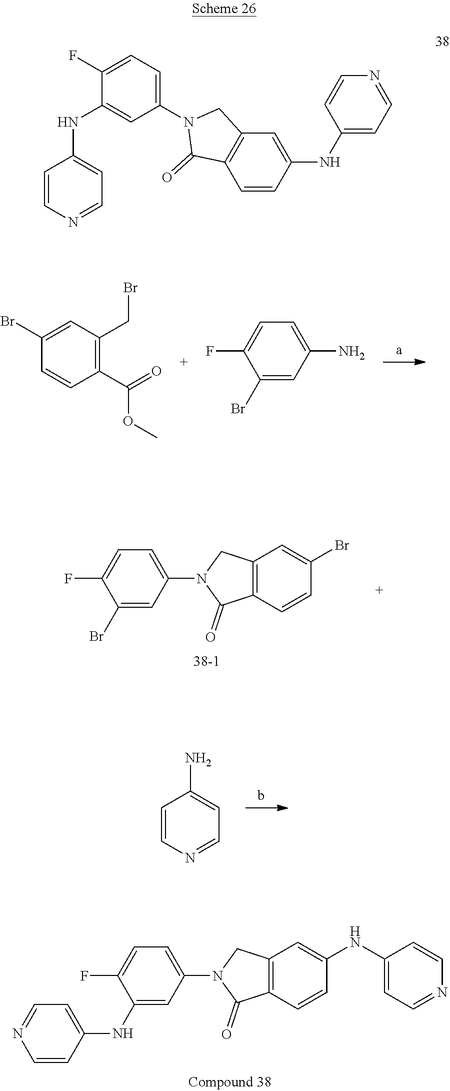 [SMILES: O=C1c2ccc(Nc3ccncc3)cc2CN1c1ccc(F)c(Nc2ccncc2)c1 ; COC(=O)c1ccc(Br)cc1CBr ; Nc1ccc(F)c(Br)c1 ; O=C1c2ccc(Br)cc2CN1c1ccc(F)c(Br)c1 ; Nc1ccncc1 ; O=C1c2ccc(Nc3ccncc3)cc2CN1c1ccc(F)c(Nc2ccncc2)c1]

Compound 38 was prepared by the method shown in Scheme 26.

Step a: To methyl 4-bromo-2-(bromomethyl)benzoate (459 mg, 1.5 mmol) was added 3-bromo-4-fluoroaniline (283 mg, 1.5 mmol), and the mixture was stirred at 135° C. for 8 hours. The reaction was quenched with MeOH (6 mL) and filtered to give Compound 38-1 as a white solid (350 mg, 60.8% yield).

Step b: To a mixture of Compound 38-1 (200 mg, 0.52 mmol) in t-BuOH (5 mL)/toluene (5 mL) was added pyridin-4-amine (147 mg, 1.56 mmol), Pd₂(dba)₃ (30 mg, 0.02 mmol), Brettphos (30 mg, 0.02 mmol), and Cs₂CO₃ (510 mg, 1.56 mmol), and the mixture was stirred at 100° C. for 12 hours under N₂. The mixture was concentrated and the crude was purified by prep-HPLC to give Compound 38 as a yellow solid (25 mg, 11% yield).

Compound 38 (2-(4-fluoro-3-(pyridin-4-ylamino)phenyl)-5-(pyridin-4-ylamino)isoindolin-1-one): C₂₄H₁₈FN₅O; 411.43 g/mol; 25 mg; white solid; ESI-LCMS m/z=412 [M+H]⁺; LCMS RT=1.62 min, >95.00% (214 nm).

### Example 39: Compound 39 (N-(2-methylpyridin-4-yl)-2-(4-((2-methylpyridin-4-yl)amino)phenyl)benzo[d]oxazol-5-amine)

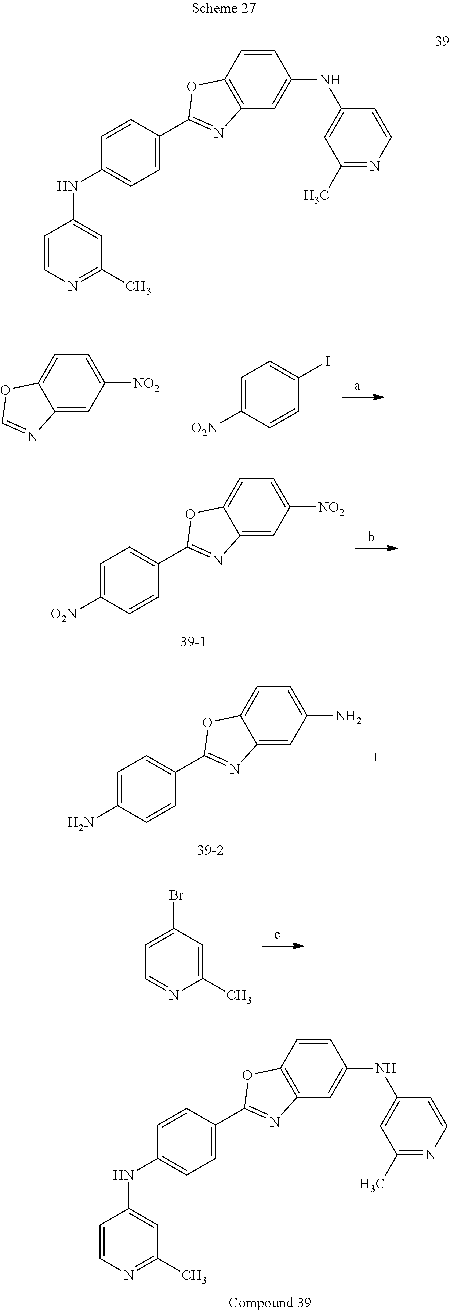

Compound 39 was prepared by the method shown in Scheme 27.

Step a: To a solution of 5-nitrobenzo[d]oxazole (500 mg, 3.05 mmol) in 1,4-dioxane (20 mL) was added 1-iodo-4-nitrobenzene (910 mg, 3.65 mmol), lithium t-butoxide (490 mg, 6.10 mmol), and Pd(PPh₃)₄ (175 mg, 0.15 mmol). The mixture was stirred at 100° C. overnight under Ar. Water (60 mL) was added and the mixture extracted with DCM (3×60 mL). The combined organic phases were washed with brine (10 mL), dried over anhydrous sodium sulfate, filtered, and concentrated in vacuo to afford crude Compound 39-1. The crude (1.2 g) was used directly in next step.

Step b: To a solution of crude Compound 39-1 (1.2 g) in ethanol (15 mL) was added a spoonful of Raney Nickel and N₂H₄·H₂O (3 mL). The mixture was stirred at room temperature for 1 hour under N₂. The mixture was filtered and the solvent was removed in vacuo. The crude was purified by prep-TLC (petroleum ether:ethyl acetate=1:1, 254 nm, silica) to afford Compound 39-2 (500 mg, 35% yield) as a brown solid.

Step c: To a solution of Compound 39-2 (140 mg, 0.62 mmol) in ethanol (6 mL) was added 4-bromo-2-methylpyridine (0.03 mL, 0.62 mmol), Pd₂dba₃ (56 mg, 0.062 mmol), K₂CO₃ (95 mg, 0.64 mmol), and X-Phos (146 mg, 0.29 mmol). The mixture was stirred at 100° C. overnight. Water (20 mL) was added and the mixture extracted with ethyl acetate (2× 20 mL). The combined organic phased were washed with brine (5 mL), dried over anhydrous sodium sulfate, filtered, and concentrated in vacuo to afford the crude product. The crude was purified by prep-HPLC to afford Compound 39 (60 mg, 63% yield) as a white solid.

Compound 39 (N-(2-methylpyridin-4-yl)-2-(4-((2-methylpyridin-4-yl)amino)phenyl)benzo[d]oxazol-5-amine): C₂₅H₂₁N₅O; 407.47 g/mol; 60 mg; white solid; ESI-LCMS m/z=408 [M+H]⁺; LCMS RT=1.67 min, >95.00% (214 and 254 nm).

### Example 40: Compound 40 (N-(2-methylpyridin-4-yl)-2-(3-((2-methylpyridin-4-yl)amino)phenyl)benzo[d]oxazol-5-amine)

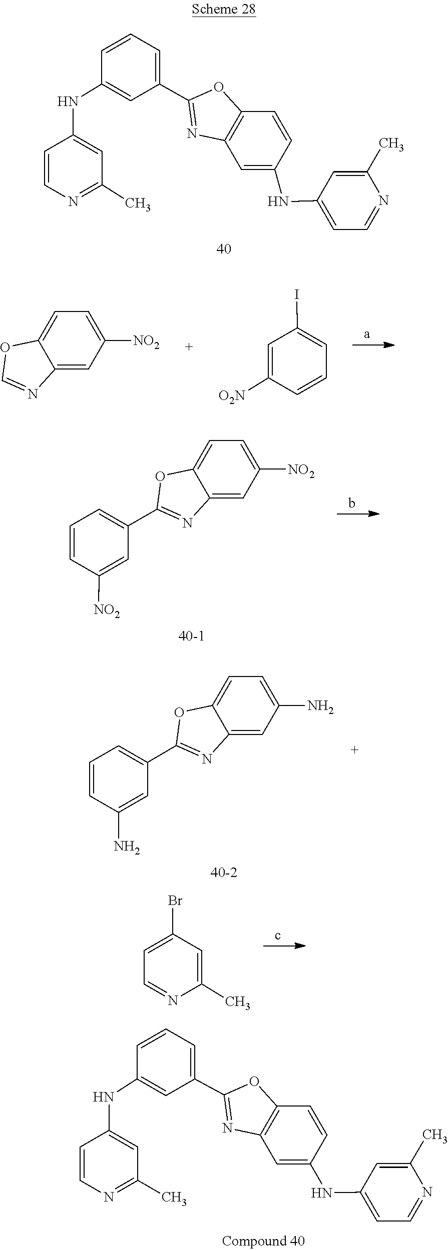

Compound 40 was prepared by the method shown in Scheme 28.

Step a: To a solution of 5-nitrobenzo[d]oxazole (500 mg, 3.05 mmol) in 1,4-dioxane (20 mL) was added 1-iodo-4-nitrobenzene (910 mg, 3.65 mmol), lithium t-butoxide (490 mg, 6.10 mmol) and Pd(PPh₃)₄ (175 mg, 0.15 mmol). The mixture was stirred at 100° C. overnight under Ar. Water (60 mL) was added and the mixture extracted with DCM (3× 60 mL). The combined organic phases were washed with brine (10 mL), dried over anhydrous sodium sulfate, filtered, and concentrated in vacuo to afford crude Compound 40-1. The crude (1.1 g) was used directly in next step.

Step b: To a solution of crude Compound 40-1 (1.1 g) in ethanol (15 mL) was added a spoonful of Raney Nickel and N₂H₄·H₂O (3 mL). The mixture was stirred at room temperature for 1 hour under N₂. The mixture was filtered and the solvent was removed in vacuo. The crude was purified by prep-TLC (petroleum ether:ethyl acetate=1:1, 254 nm, silica) to afford Compound 40-2 (475 mg, 33% yield) as a brown solid.

Step c: To a solution of Compound 40-2 (250 mg, 1.1 mmol) in ethanol (60 mL) was added 4-bromo-2-methylpyridine (0.06 mL, 1.1 mmol), Pd₂dba₃ (100 mg, 0.11 mmol), K₂CO₃ (170 mg, 1.2 mmol), and X-Phos (261 mg, 0.55 mmol). The mixture was stirred at 100° C. overnight. Water (150 mL) was added and the mixture extracted with ethyl acetate (2×150 mL). The combined organic phases were washed with brine (20 mL), dried over anhydrous sodium sulfate, filtered, and concentrated in vacuo to afford the crude product. The crude was purified by prep-HPLC to afford Compound 40 (80 mg, 67% yield) as a white solid.

Compound 40 (N-(2-methylpyridin-4-yl)-2-(3-((2-methylpyridin-4-yl)amino)phenyl)benzo[d]oxazol-5-amine): C₂₅H₂₁N₅O; 407.47 g/mol; 80 mg; white solid; ESI-LCMS m/z=408 [M+H]⁺; LCMS RT=1.75 min, >95.00% (214 and 254 nm).

### Example 41: Compound 41 (N-(2-methylpyridin-4-yl)-2-(4-((2-methylpyridin-4-yl)amino)phenyl)benzo[d]oxazol-6-amine)

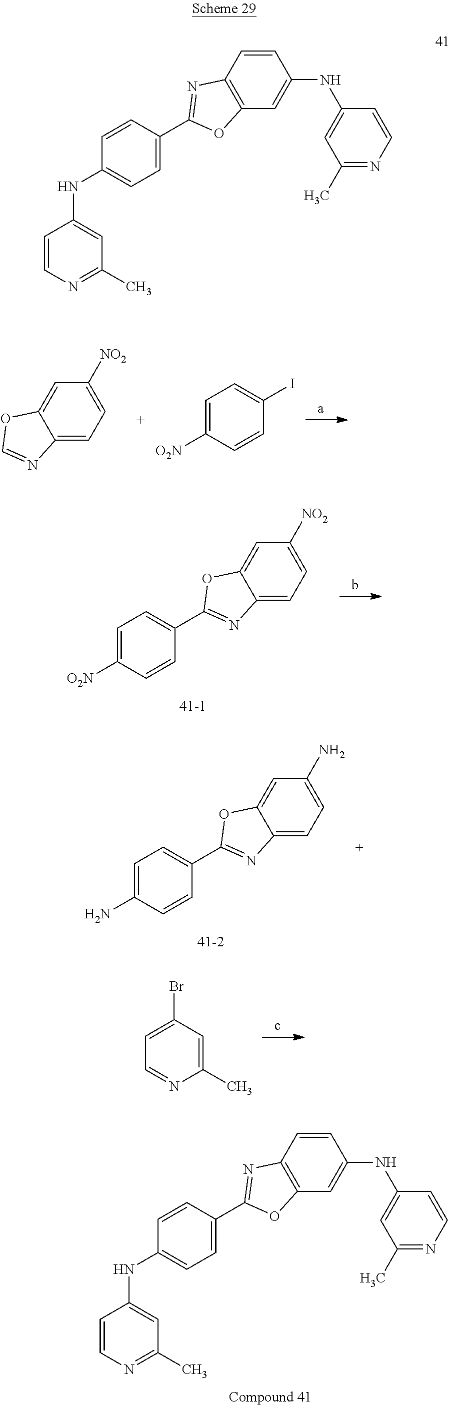

Compound 41 was prepared by the method shown in Scheme 29.

Step a: To a solution of 6-nitrobenzo[d]oxazole (500 mg, 3.05 mmol) in 1,4-dioxane (20 mL) was added 1-iodo-4-nitrobenzene (910 mg, 3.65 mmol), lithium t-butoxide (490 mg, 6.10 mmol), and Pd(PPh₃)₄ (175 mg, 0.15 mmol). The mixture was stirred at 100° C. overnight under Ar. Water (60 mL) was added and the mixture extracted with DCM (3×60 mL). The combined organic phases were washed with brine (10 mL), dried over anhydrous sodium sulfate, filtered, and concentrated in vacuo to afford crude Compound 41-1. The crude (1.5 g) was used directly in next step.

Step b: To a solution of crude Compound 41-1 (1.5 g) in ethanol (15 mL) was added a spoonful of Raney Nickel and N₂H₄·H₂O (3 mL). The mixture was stirred at room temperature for 1 hour under N₂. The mixture was filtered and the solvent was removed in vacuo. The crude was purified by prep-TLC (petroleum ether:ethyl acetate=1:1, 254 nm, silica) to afford Compound 41-2 (321 mg, 30% yield) as a brown solid.

Step c: To a solution of Compound 41-2 (170 mg, 0.60 mmol) in ethanol (8 mL) was added 4-bromo-2-methylpyridine (0.04 mL, 0.60 mmol), Pd₂dba₃ (68 mg, 0.060 mmol), K₂CO₃ (116 mg, 0.71 mmol), and X-Phos (177 mg, 0.32 mmol). The mixture was stirred at 100° C. overnight. Water (20 mL) was added and the mixture extracted with ethyl acetate (2×20 mL). The combined organic phases were washed with brine (5 mL), dried over anhydrous sodium sulfate, filtered, and concentrated in vacuo to afford the crude product. The crude was purified by prep-HPLC to afford Compound 41 (32 mg, 57% yield) as a white solid.

Compound 41 (N-(2-methylpyridin-4-yl)-2-(4-((2-methylpyridin-4-yl)amino)phenyl)benzo[d]oxazol-6-amine): C₂₅H₂₁N₅O; 407.47 g/mol; 32 mg; white solid; ESI-LCMS m/z=408 [M+H]⁺; LCMS RT=1.65 min, >95.00% (214 and 254 nm).

### Example 42: Compound 42 (N-(2-methylpyridin-4-yl)-2-(3-((2-methylpyridin-4-yl)amino)phenyl)benzo[d]oxazol-6-amine)

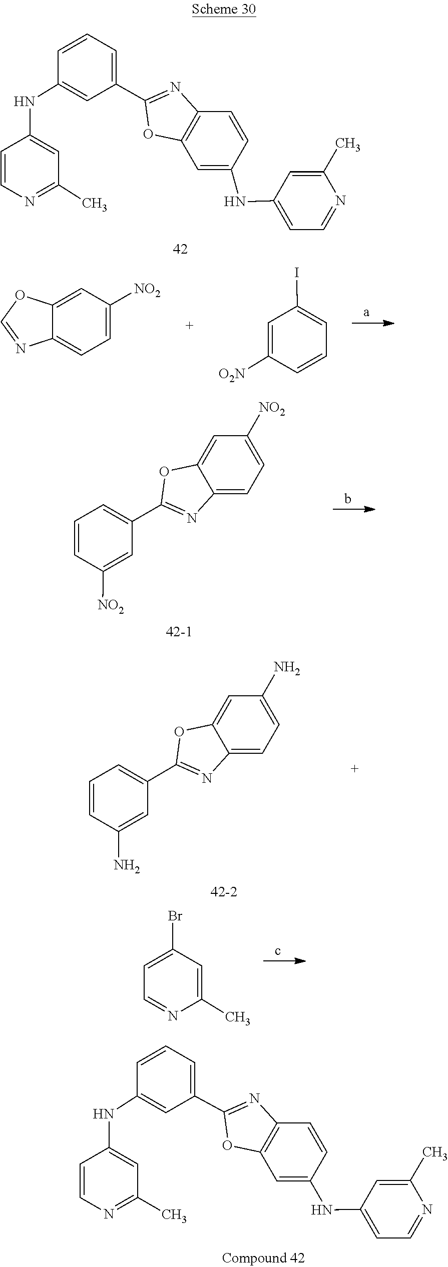

Compound 42 was prepared by the method shown in Scheme 30.

Step a: To a solution of 6-nitrobenzo[d]oxazole (500 mg, 3.05 mmol) in 1,4-dioxane (20 mL) was added 1-iodo-4-nitrobenzene (910 mg, 3.65 mmol), lithium t-butoxide (490 mg, 6.10 mmol) and Pd(PPh₃)₄ (175 mg, 0.15 mmol). The mixture was stirred at 100° C. overnight under Ar. Water (60 mL) was added and the mixture extracted with DCM (3×60 mL). The combined organic phases were washed with brine (10 mL), dried over anhydrous sodium sulfate, filtered, and concentrated in vacuo to afford crude Compound 42-1. The crude (1.2 g) was used directly in next step.

Step b: To a solution of crude Compound 42-1 (1.2 g) in ethanol (15 mL) was added a spoonful of Raney Nickel and N₂H₄·H₂O (3 mL). The mixture was stirred at room temperature for 1 hour under N₂. The mixture was filtered and the solvent was removed in vacuo. The crude was purified by prep-TLC (petroleum ether:ethyl acetate=1:1, 254 nm, silica) to afford Compound 42-2 (563 mg, 42% yield) as a brown solid.

Step c: To a solution of Compound 42-2 (250 mg, 1.1 mmol) in ethanol (60 mL) was added 4-bromo-2-methylpyridine (0.06 mL, 1.1 mmol), Pd₂dba₃ (100 mg, 0.11 mmol), K₂CO₃ (170 mg, 1.2 mmol), and X-Phos (261 mg, 0.55 mmol). The mixture was stirred at 100° C. overnight. Water (150 mL) was added and the mixture extracted with ethyl acetate (2×150 mL). The combined organic phases were washed with brine (20 mL), dried over anhydrous sodium sulfate, filtered, and concentrated in vacuo to afford the crude product. The crude was purified by prep-HPLC to afford Compound 42 (40 mg, 53% yield) as a white solid.

Compound 42 (N-(2-methylpyridin-4-yl)-2-(3-((2-methylpyridin-4-yl)amino)phenyl)benzo[d]oxazol-6-amine): C₂₅H₂₁N₅O; 407.47 g/mol; 40 mg; white solid; ESI-LCMS m/z=408 [M+H]⁺; LCMS RT=1.53 min, >95.00% (214 nm).

### Example 43: Compound 43 (2-(3-((4-fluorophenyl)amino)phenyl)-N-(2-methylpyridin-4-yl)benzo[d]oxazol-5-amine)

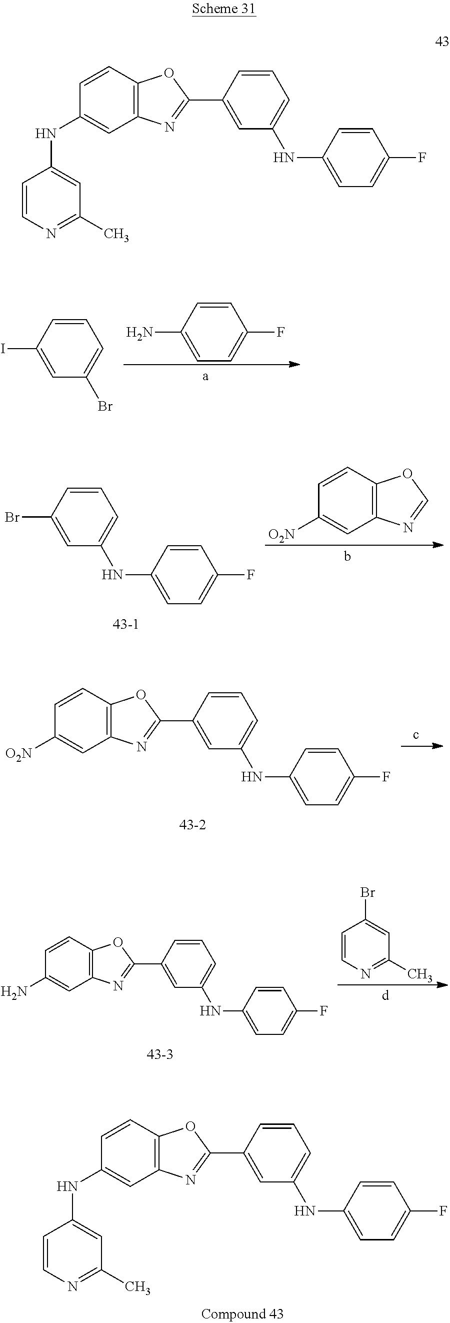

Compound 43 was prepared by the method shown in Scheme 31.

Step a: To a solution of 1-bromo-3-iodobenzene (200 mg, 0.7 mmol) in xylene (10 mL) was added 4-fluoroaniline (40 mg, 0.36 mmol), Pd(PPh₃)₂Cl₂ (25 mg, 0.036 mmol), PPh (10 mg, 0.07 mmol), and t-BuONa (120 mg, 1.08 mmol) under Ar. The mixture was stirred at 140° C. for 16 hours under Ar. The mixture was concentrated and the residue was purified by EA/PE (0%-10%) to give Compound 43-1 (150 mg, 80% yield) as a yellow solid.

Step b: To a solution of Compound 43-1 (160 mg, 0.6 mmol) in 1,4-dioxane (5 mL) was added 5-nitrobenzo[d]oxazole (75 mg, 0.5 mmol), Pd(Ph₃)₄ (57 mg, 0.05 mmol) and t-BuLi (60 mg, 1.0 mmol) at room temperature under Ar. The mixture was stirred at 100° C. under Ar overnight. The mixture was concentrated and the residue was purified by EA/PE (0%-10%) to give Compound 43-2 (50 mg, 50% yield) as a yellow solid.

Step c: To a solution of Compound 43-2 (50 mg, 0.28 mmol) in EtOH (6 mL) and DCM (3 mL) was added NH₂NH₂·H₂O (14 mg, 0.28 mmol) and Raney-Ni (10 mg) at room temperature under H₂. The mixture was stirred at room temperature for 16 hours. The mixture was filtered. The filtrate was concentrated to give Compound 43-3 (50 mg, 100% yield) as a yellow solid.

Step d: To a solution of Compound 43-3 (100 mg, 0.31 mmol) in EtOH (3 mL) was added 4-bromo-2-methylpyridine (100 mg, 0.6 mmol), X-phos (74 mg, 0.062 mmol), Pd₂(dba)₃ (30 mg, 0.031 mmol), and K₂CO₃ (200 mg, 1.55 mmol) at room temperature under Ar. The mixture was stirred at 100° C. under Ar overnight. The reaction was cooled to room temperature and filtered. The filtrate was concentrated under reduced pressure. The residue was purified by prep-HPLC (NH₄HCO₃/H₂O/MeCN) to give Compound 43 (38.2 mg, 30% yield) as a yellow solid.

Compound 43 (2-(3-((4-fluorophenyl)amino)phenyl)-N-(2-methylpyridin-4-yl)benzo[d]oxazol-5-amine): C₂₅H₁₉FN₄O; 410.45 g/mol; 38.2 mg; yellow solid; ESI-LCMS m/z=411 [M+H]⁺; LCMS RT=1.106 min, >95.00% (214 and 254 nm).

### Example 44: Compound 44 (7-methyl-5-((2-methylpyridin-4-yl)amino)-2-(3-(pyridin-4-ylamino)phenyl)isoindolin-1-one)

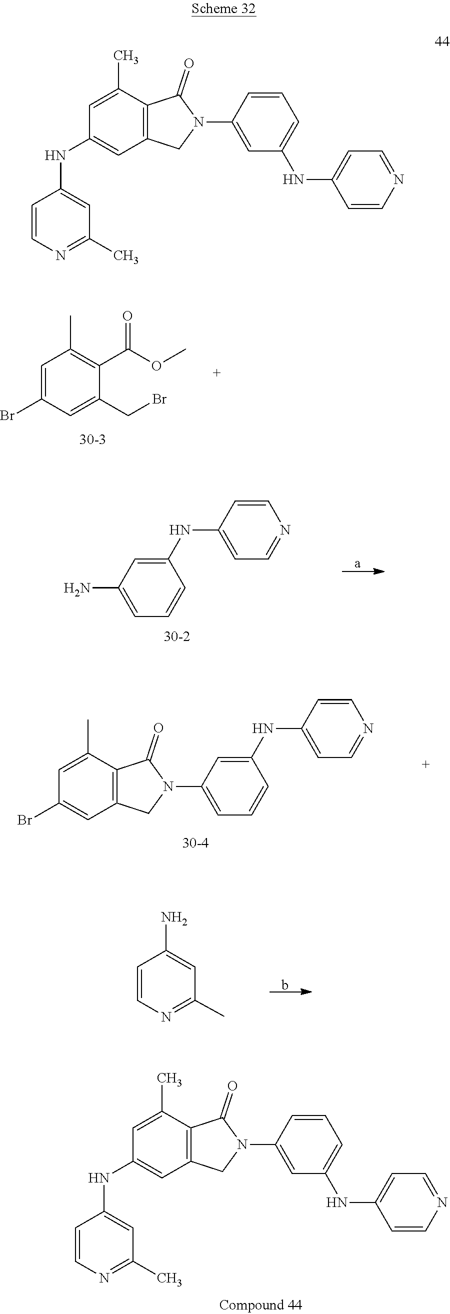

Compound 44 was prepared by the method shown in Scheme 32.

Step a: To a mixture of Compound 30-3 (640 mg, 2 mmol) and Compound 30-2 (407 mg, 2.2 mmol) in MeOH (10 mL) was added TEA (606 mg, 6 mmol), and the mixture was stirred at 85° C. for 16 hours. The combined organic phases were concentrated. The residue was purified by flash chromatography on silica gel (0-20% MeOH in DCM) to afford Compound 30-4 (400 mg, 50.8% yield) as a solid.

Step b: To a mixture of Compound 30-4 (150 mg, 0.38 mmol) in t-BuOH (5 mL)/toluene (5 mL) was added 2-methylpyridin-4-amine (50 mg, 0.45 mmol), Pd₂(dba)₃ (20 mg, 0.02 mmol), Brettphos (20 mg, 0.02 mmol), and Cs₂CO₃ (247 mg, 0.76 mmol), and the mixture was stirred at 100° C. for 12 hours under N₂. The mixture was concentrated and the crude was purified by prep-HPLC to give Compound 44 as a yellow solid (15 mg, 10% yield).

Compound 44 (7-methyl-5-((2-methylpyridin-4-yl)amino)-2-(3-(pyridin-4-ylamino)phenyl)isoindolin-1-one): C₂₆H₂₃N₅O; 421.49 g/mol; 15 mg; yellow solid; ESI-LCMS m/z=422 [M+H]⁺; LCMS RT=1.30 min, >95.00% (214 and 254 nm).

### Example 45: Compound 45 (7-methyl-5-((2-methylpyridin-4-yl)amino)-2-(3-((2-methylpyridin-4-yl)amino)phenyl)isoindolin-1-one)

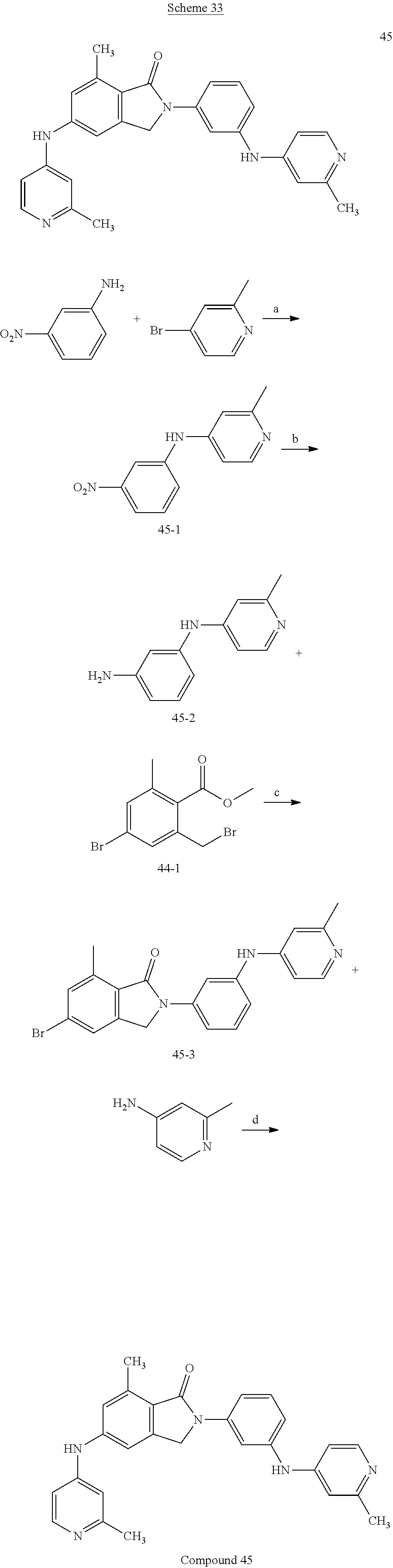

Compound 45 was prepared by the method shown in Scheme 33.

Step a: To a stirred mixture of 3-nitroaniline (2.76 g, 0.02 mol) in 1,4-dioxane (30 mL) was added 4-bromo-2-methylpyridine (3.42 g, 0.02 mol), Cs₂CO₃ (13 g, 0.04 mol), Pd₂(dba)₃ (280 mg, 0.25 mmol), and Xantphos (280 mg, 0.4 mmol) under N₂. The resulting mixture was stirred at 100° C. for 3 hours. The reaction was quenched with water (60 mL) and extracted with EtOAc (3× 50 mL). The combined organic phases were dried over Na₂SO₄, filtered, and concentrated. The residue was purified by flash chromatography on silica gel (0-50% EtOAc in petroleum ether) to afford Compound 45-1 (4 g, 87% yield) as a yellow solid.

Step b: To a mixture of Compound 45-1 (4 g, 17.5 mmol) in MeOH (100 mL) was added Pd/C (400 mg), and the mixture was stirred at room temperature for 4 hours under H₂. The combined organic phases were filtered by diatomite to give Compound 45-2 as a yellow solid (3 g, 88.7% yield).

Step c: To a mixture of Compound 30-3 (400 mg, 1.25 mmol) and Compound 45-2 (273 mg, 1.3 mmol) in MeOH (10 mL) was added TEA (253 mg, 2.5 mmol), and the mixture was stirred at 85° C. for 16 hours. The combined organic phases were concentrated. The residue was purified by flash chromatography on silica gel (0-20% MeOH in DCM) to afford Compound 45-3 (300 mg, 59% yield) as a solid.

Step d: To a mixture of Compound 45-3 (150 mg, 0.36 mmol) in t-BuOH (5 mL)/toluene (5 mL) was added 2-methylpyridin-4-amine (39 mg, 0.45 mmol), Pd₂(dba)₃ (20 mg, 0.02 mmol), Brettphos (20 mg, 0.02 mmol), and Cs₂CO₃ (234 mg, 0.72 mmol), and the mixture was stirred at 100° C. for 12 hours under N₂. The mixture was concentrated and the crude was purified by prep-HPLC to give Compound 45 as a yellow solid (18 mg, 11% yield).

Compound 45 (7-methyl-5-((2-methylpyridin-4-yl)amino)-2-(3-((2-methylpyridin-4-yl)amino)phenyl)isoindolin-1-one): C₂₇H₂₅N₅O; 435.53 g/mol; 18 mg; yellow solid; ESI-LCMS m/z=436 [M+H]⁺; LCMS RT=1.41 min, >95.00% (214 and 254 nm).

### Example 46. Biological Assays

### Foxp3 Induction Assay

Sorted or enriched (Miltenyi magnetic separation) CD4 conventional T cells (Tconvs—CD4+/CD25) from C₅₇/B16 mice were used for the induction of iTregs. A 10 μg/mL plate-bound anti-CD3 antibody (50 μl per well for 96-well plate), 2.5 μg/mL of soluble anti-CD28 antibody, 100 IU/mL of IL2 and 5 ng/ml of TGF-β in absence or presence of different concentrations of drug (usually titrating from 0.01 μM to 10 μM) were used. As negative control for induction, samples without TGF-β were used.

After 3 days of culture in presence of stimulation, TGF-β and drug, cells were stained with fixable live/dead cell stain (Life Technologies, NY) for gating and exclusion of toxic doses. The mouse Foxp3 buffer kit was used to fix and permeabilize cells according to the manufacturer's instructions (BD Bioscience, San Jose, CA). The anti-CD4 antibody and anti-Foxp3 antibody were used to stain the cells. After staining, cells were acquired using flow cytometer.

### Jurkat-FoxP3 Reporter Assay (According to BPS Bioscience, Cat #60628)

Cells Culture Process: Prepare a 50 ml conical tube and a T-25 culture flask with 5 ml of pre-warmed Thaw Medium 2 (no G418). Quickly thaw cells in a 37° C. water bath with constant and slow agitation. Immediately transfer the entire contents to the conical tube with Thaw Medium 2 (no G418) and centrifuge the cells at 200×g for 3 minutes. Re-suspend the cells in 6 ml of pre-warmed Thaw Medium 2 (no G418) and transfer the entire content to the T25 culture flask containing Thaw Medium 2 (no G418). Incubate the cells in a humidified 37° C. incubator with 5% CO2. Forty-eight hours after incubation, centrifuge cells at 250×g for 5 minutes and re-suspend to fresh Thaw Medium 2 (no G418). Continue to monitor growth for 2-3 days and change medium to remove dead debris. Switch to Growth Medium 2B (containing G418) after multiple cell colonies (in clumps) start to appear (indicative of healthy cell division)

### After Assay Protocol: (CD3/CD28)

1. In a white opaque 384-well plate, Jurkat-FoxP3-luciferase reporter cells at ˜2.5×103 cells/well (10 μL per well) in Assay Medium (RPMI 1640 medium (Thermo Fisher, Cat. #A1049101) supplemented with 1% Penicillin/Streptomycin) were cultured in absence and presence of (ratio: 1:5) of Human T-Activator CD3/CD28 Dynabead (Thermo Fisher, Cat. No. 11161D).

2. Make drugs serial dilution range 1-60,000 nM and add 10 μL of drugs, which will yield a range of 1-30,000 nM, and mix with gentle sacking. In some experiments, the range is from 10-20,000 nM. Cells were cultured in presence and absence of drugs for 12 hours at 37° C. with 5% CO₂.

3. Add ONE-Step™ Luciferase Assay System (BPS Bioscience, Cat. #60690) to each well, according to the protocol. Add equal volume of luciferase assay working solution (Component A+Component B) to the culture medium in each well. As an example, a 384 well plate with 20 μl of culture medium requires 20 μl of luciferase assay working solution per well.

4. Gently rock the plates for ≥15 minutes at room temperature. Measure firefly luminescence using a luminometer.

### Phospho-Akt Isoform Specificity Assay

Human CD4+/CD45RA+/CD25−naïve T cells were plated under induction conditions (IL-2/anti-CD3/anti-CD28+TGFβ) in the absence or presence of compounds for 72 hours. To determine the compounds' specificity for each phospho-AKT isoform, phospho-AKT cellular HTRF kits (Cisbio catalogue numbers 63ADK078PEG (pAKT1), 63ADK080PEG (p-AKT2), and 63ADK082PEG (pAKT3)) were used according to manufacturer specifications. Briefly, after removal of the supernatant, cells were lysed, and total protein concentration measured and normalized for all samples. The cell lysates were transferred into 384-well plates and Eu Cryptate antibody+d2 antibody mixture was added. This process was the same for each isoform but utilized the corresponding isoform antibodies from each respective kit. Positive and negative controls (supplied with the kit) were incorporated into each experiment. The plates were incubated overnight. Data acquisition was performed on the Varioskan Lux reader utilizing the settings for the TRF fluorescence protocol. Data was presented as percent change over DMSO-treated controls. Each test condition was run in duplicate, and the assay was performed at least twice.

### IL-10 ELISA Assay

Human CD4+/CD25+natural Treg cells were plated under stimulating conditions (IL-2/anti-CD3/anti-CD28) in the absence or presence of compounds. 24 and 48 hours after incubation, the supernatants were collected, and IL-10 concentrations were determined using the Human IL-10 ELISA kit according to manufacturer specifications (Invitrogen BMS215-2). Briefly, supernatants were added to pre-coated 96-well ELISA plates and incubated, followed by addition of biotin-conjugated detection antibodies and Streptavidin-HRP. After incubation, substrate was added, and the reaction was stopped by addition of acid. Absorbance was measured at 450 nm using the Varioskan Lux reader. Known concentrations of IL-10 (provided in the kit) were used to generate the calibration curves and calculate the concentration of IL-10 in supernatants. Data was presented as percent change over untreated stimulated cell controls. Each test condition was run in triplicate, and the assay was performed at least twice.

### Foxp3 ELISA Assay

Human CD4+/CD45RA+/CD25-naïve T cells were plated under induction conditions (IL-2/anti-CD3/anti-CD28+TGFβ) in absence or presence of compounds for 72 hours. After incubation, cells were lysed and FoxP3 protein was measured in lysates using the Human FoxP3 ELISA kit according to manufacturer specifications (LSBio, LS-F5047). Briefly, lysates were added to pre-coated 96-well ELISA plates and incubated, followed by biotin-conjugated detection antibodies and Streptavidin-HRP. After incubation, substrate was added, and the reaction was stopped by addition of acid. Absorbance was measured at 450 nm using the Varioskan Lux reader. Known concentrations of FoxP3 (provided in the kit) were used to generate the calibration curves and calculate the concentration of FoxP3 in lysates. Data was presented as percent change over cells induced in the absence of compounds. Each test condition was run in duplicate, and the assay was performed at least twice.

iTreg Induction Assay

Sorted human CD4 T cells were used for the induction of iTregs. Human T cell activation beads (Gibco Dynabeads CD3/CD28), 100 IU/mL of IL2 and 5 ng/ml of TGF-β, in absence or presence of different concentrations of drug, were used. As negative control for induction, samples without TGF-β were used. After 3 days of culture in the presence of stimulation with TGF-β and drug, cells were stained with fixable live/dead cell stain (Life Technologies) for gating and exclusion of toxic doses, fixed and permeabilized using the Foxp3 buffer kit according to the manufacturer specifications (BD Bioscience), and stained with anti-Foxp3 antibody. After staining, cells were acquired using flow cytometer. Each test condition was run in duplicate, and the assay was performed at least twice.

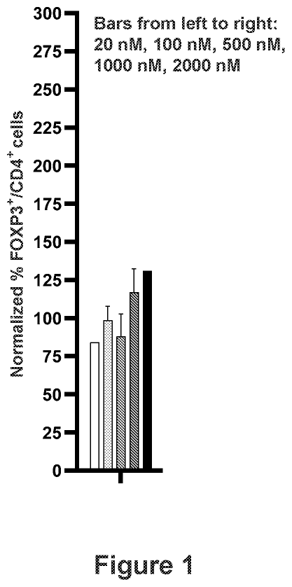

Data illustrated by FIG. 1 was obtained at least partially using this assay protocol. FIG. 1 shows evaluation of iTreg induction (FoxP3) from human CD4 T cells treated with Compound 22 in the presence of anti-CD3/anti-CD28/IL-2/TGFβ.

The Akt3 inhibition and activation activities of selected compounds disclosed herein are shown in Tables 1 and 2, respectively.

TABLE 1
Akt3 inhibition activity of selected compound.

| Compound | | IC₅₀ |
|---|---|---|
| No. | Structure | (μM) |
| 3 | 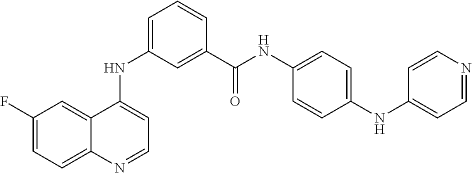 [SMILES: O=C(Nc1ccc(Nc2ccncc2)cc1)c1cccc(Nc2ccnc3ccc(F)cc23)c1] | <2 |
| 18 | 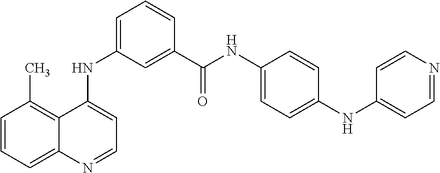 [SMILES: Cc1cccc2nccc(Nc3cccc(C(=O)Nc4ccc(Nc5ccncc5)cc4)c3)c12] | <2 |
| 19 | 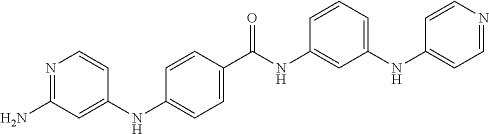 [SMILES: Nc1cc(Nc2ccc(C(=O)Nc3cccc(Nc4ccncc4)c3)cc2)ccn1] | <5 |
| 20 | 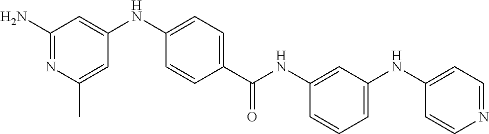 [SMILES: Cc1cc(Nc2ccc(C(=O)Nc3cccc(Nc4ccncc4)c3)cc2)cc(N)n1] | <5 |
| 21 | 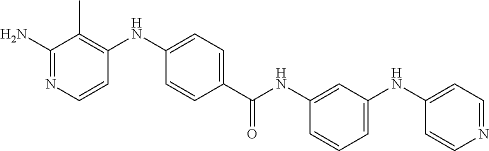 [SMILES: Cc1c(Nc2ccc(C(=O)Nc3cccc(Nc4ccncc4)c3)cc2)ccnc1N] | <2 |
| 26 | 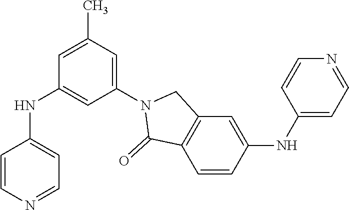 [SMILES: Cc1cc(Nc2ccncc2)cc(N2Cc3cc(Nc4ccncc4)ccc3C2=O)c1] | <1 |
| 27 | 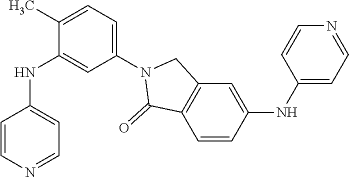 [SMILES: Cc1ccc(N2Cc3cc(Nc4ccncc4)ccc3C2=O)cc1Nc1ccncc1] | <1 |
| 30 | 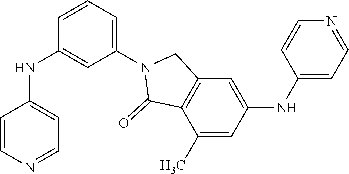 [SMILES: Cc1cc(Nc2ccncc2)cc2c1C(=O)N(c1cccc(Nc3ccncc3)c1)C2] | <1 |
| 31 | 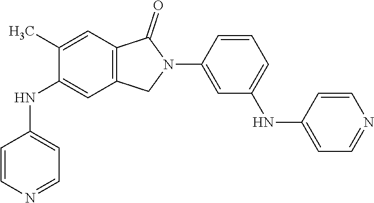 [SMILES: Cc1cc2c(cc1Nc1ccncc1)CN(c1cccc(Nc3ccncc3)c1)C2=O] | <5 |
| 32 | 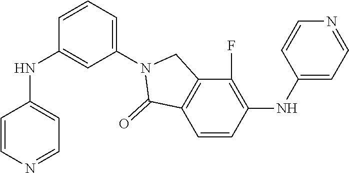 [SMILES: O=C1c2ccc(Nc3ccncc3)c(F)c2CN1c1cccc(Nc2ccncc2)c1] | <5 |
| 33 | 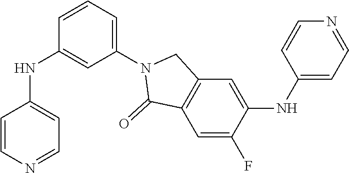 [SMILES: O=C1c2cc(F)c(Nc3ccncc3)cc2CN1c1cccc(Nc2ccncc2)c1] | <1 |
| 34 | 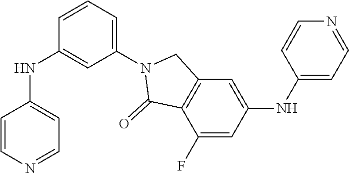 [SMILES: O=C1c2c(F)cc(Nc3ccncc3)cc2CN1c1cccc(Nc2ccncc2)c1] | <5 |
| 37 | 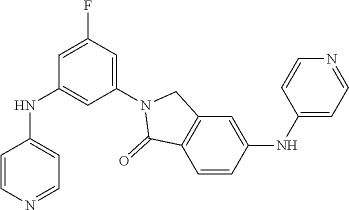 [SMILES: O=C1c2ccc(Nc3ccncc3)cc2CN1c1cc(F)cc(Nc2ccncc2)c1] | <2 |
| 38 | 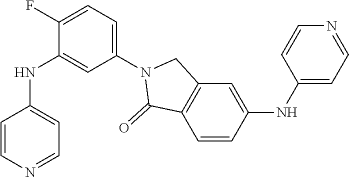 [SMILES: O=C1c2ccc(Nc3ccncc3)cc2CN1c1ccc(F)c(Nc2ccncc2)c1] | <1 |
| 39 | 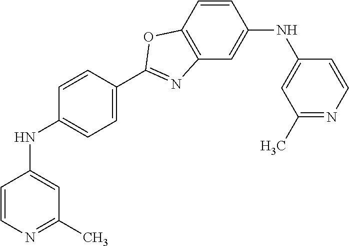 [SMILES: Cc1cc(Nc2ccc(-c3nc4cc(Nc5ccnc(C)c5)ccc4o3)cc2)ccn1] | <5 |
| 40 | 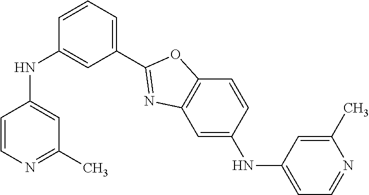 [SMILES: Cc1cc(Nc2cccc(-c3nc4cc(Nc5ccnc(C)c5)ccc4o3)c2)ccn1] | <2 |
| 41 | 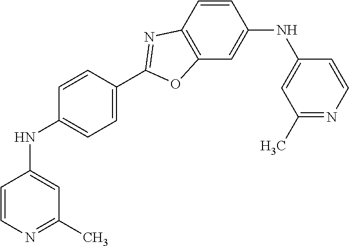 [SMILES: Cc1cc(Nc2ccc(-c3nc4ccc(Nc5ccnc(C)c5)cc4o3)cc2)ccn1] | <5 |
| 42 | 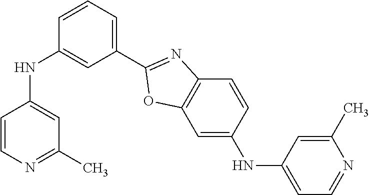 [SMILES: Cc1cc(Nc2cccc(-c3nc4ccc(Nc5ccnc(C)c5)cc4o3)c2)ccn1] | <5 |
| 43 | 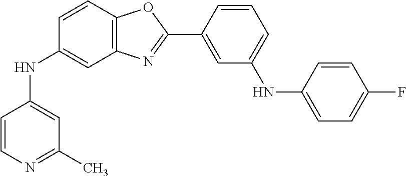 [SMILES: Cc1cc(Nc2ccc3oc(-c4cccc(Nc5ccc(F)cc5)c4)nc3c2)ccn1] | <5 |
| 44 | 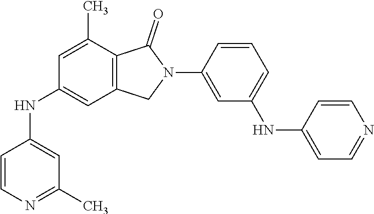 [SMILES: Cc1cc(Nc2cc(C)c3c(c2)CN(c2cccc(Nc4ccncc4)c2)C3=O)ccn1] | <1 |
| 45 | 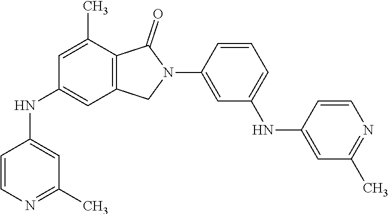 [SMILES: Cc1cc(Nc2cccc(N3Cc4cc(Nc5ccnc(C)c5)cc(C)c4C3=O)c2)ccn1] | <1 |

TABLE 2
Akt3 activation activity of selected compound.

| Compound | | EC₅₀ |
|---|---|---|
| No. | Structure | (μM) |
| 2 | 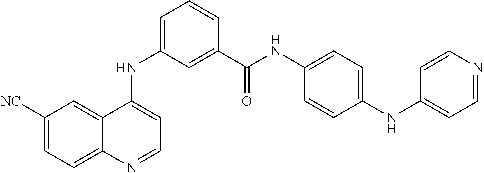 [SMILES: N#Cc1ccc2nccc(Nc3cccc(C(=O)Nc4ccc(Nc5ccncc5)cc4)c3)c2c1] | <5 |

1. A compound of Formula Ia, Ib, or Ic, 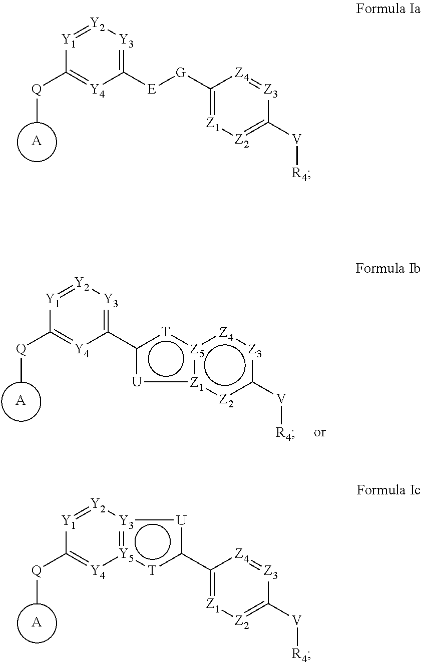
or a pharmaceutically acceptable salt thereof,
wherein:
\textcircled{A} is 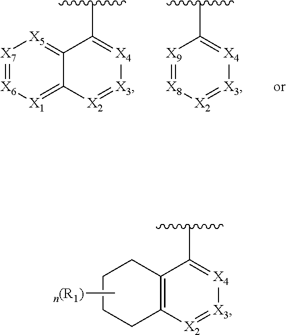
each occurrence of X₁, X₂, X₃, X₄, X₅, X₆, X₇, X₈, and X₉ is independently CR₁ or N;
each occurrence of R₁ is independently selected from the group consisting of H, D, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₇)cycloalkyl, (C₄-C₁₀)bicycloalkyl, (C₄-C₁₄)tricycloalkyl, (C₃-C₇)heterocycloalkyl, halogenated (C₃-C₇)heterocycloalkyl, (C₄-C₁₀)heterobicycloalkyl, (C₄-C₁₄)heterotricycloalkyl, (C₄-C₁₀)heterospiroalkyl, (C₃-C₇)cycloalkenyl, (C₃-C₇)heterocycloalkenyl, (C₄-C₁₀)bicycloalkenyl, (C₄-C₁₀)heterobicycloalkenyl, (C₄-C₁₄)tricycloalkenyl, (C₄-C₁₄)heterotricycloalkenyl, aryl, heteroaryl, —ORₐ, —SRₐ, —N(Rₐ)₂, —CORₐ, —CO₂Rₐ, CON(Rₐ)₂, —CN, —NC, NO₂, N₃, —SO₂Rₐ, —SO₂N(Rₐ)₂, —N(Rₐ)SO₂Rₐ, 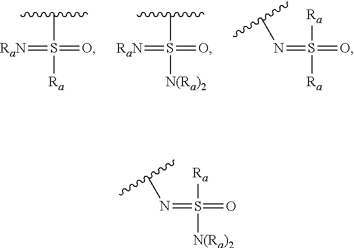
and a partially saturated bicyclic heteroaryl optionally substituted by one or more (C₁-C₆)alkyl, halogenated (C₁-C₆)alkyl, —SO₂Rₐ, or —SO₂N(Rₐ)₂;
wherein the (C₃-C₇)cycloalkyl, (C₄-C₁₀)bicycloalkyl, (C₄-C₁₄)tricycloalkyl, (C₃-C₇)heterocycloalkyl, halogenated (C₃-C₇)heterocycloalkyl, (C₄-C₁₀)heterobicycloalkyl, (C₄-C₁₄)heterotricycloalkyl, (C₄-C₁₀)heterospiroalkyl, (C₃-C₇)cycloalkenyl, (C₃-C₇)heterocycloalkenyl, (C₄-C₁₀)bicycloalkenyl, (C₄-C₁₀)heterobicycloalkenyl, (C₄-C₁₄)tricycloalkenyl, (C₄-C₁₄)heterotricycloalkenyl, aryl, and heteroaryl of R₁ are each optionally substituted by one or more (C₁-C₆)alkyl, halogenated (C₁-C₆)alkyl, halogen, —ORₐ, —CN, or —N(Rₐ)₂;
n is an integer from 0-4 where valence permits;
Q is C(Rₐ)₂, O, NRₐ, N(C═O)Rₐ, or NSO₂Rₐ;
Y₁, Y₂, Y₃, Y₄ and Y₅ are each independently N or CR₂ where valance permits;
R₂ is selected from the group consisting of H, halogen, D, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₇)cycloalkyl, (C₄-C₁₀)bicycloalkyl, (C₃-C₇)heterocycloalkyl, halogenated (C₃-C₇)heterocycloalkyl, (C₄-C₁₀)heterobicycloalkyl, (C₄-C₁₀)heterospiroalkyl, aryl, heteroaryl, —ORₐ, —SRₐ, —N(Rₐ)₂, —CORₐ, —CO₂Rₐ, CON(Rₐ)₂, —CN, —NC, NO₂, N₃, —SO₂Rₐ, —SO₂N(Rₐ)₂, —N(Rₐ)SO₂Rₐ, 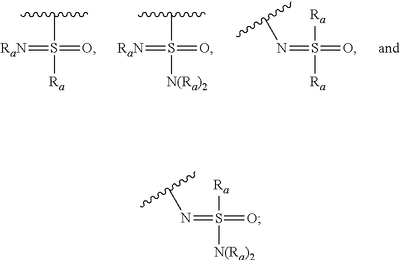
-E-G- is —(C═O)NRₓ—, —NRₓ(C═O)—, —N(Rₓ)(C═O)N(Rₓ)—, —O(C═O)N(Rₓ)—, —N(Rₓ)(C═O)O—, —SO₂NRₓ—, —NRₓSO₂—, or 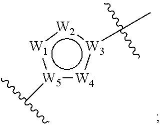
wherein;
each occurrence of Rₓ is independently H, (C₁-C₆)alkyl, (C₃-C₇)cycloalkyl, aryl, or heteroaryl; or wherein Rₓ and Y₃, Rₓ and Y₄, Rₓ and Z₁, or Rₓ and Z₄ taken together form a 5-6-membered heterocycle optionally substituted by one or more R_{z}, wherein each occurrence of R_{z} is independently H, (C₁-C₆)alkyl, OH, O(C₁-C₆)alkyl, or halogen; or two R_{z} taken together form a 4-6-membered ring optionally substituted with halogen or (C₁-C₆)alkyl;
W₁, W₂, W₃, W₄, and W₅ are each independently CR₆, N, or NR₆ where valence permits;
each occurrence of R₆ is independently selected from the group consisting of H, halogen, (C₁-C₆)alkyl, and (C₁-C₆)haloalkyl;
each occurrence of T is independently O, N, NRₐ, N(C═O)Rₐ, NC(R_{b})₂OP(═O)(OR_{b})₂, or NSO₂Rₐ where valance permits;
each occurrence of U is independently O, N, NRₐ, N(C═O)Rₐ, NC(R_{b})₂OP(═O)(OR_{b})₂, or NSO₂Rₐ where valance permits;
each occurrence of R_{b} is independently H or (C₁-C₆)alkyl;
Z₁, Z₂, Z₃, Z₄ and Z₅ are each independently N or CR₃ where valance permits;
R₃ is selected from the group consisting of H, D, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₇)cycloalkyl, (C₄-C₁₀)bicycloalkyl, (C₃-C₇)heterocycloalkyl, halogenated (C₃-C₇)heterocycloalkyl, (C₄-C₁₀)heterobicycloalkyl, (C₄-C₁₀)heterospiroalkyl, aryl, heteroaryl, —ORₐ, —SRₐ, —N(Rₐ)₂, —CORₐ, —CO₂Rₐ, CON(Rₐ)₂, —CN, —NC, NO₂, N₃, —SO₂Rₐ, —SO₂N(Rₐ)₂, —N(Rₐ)SO₂Rₐ, 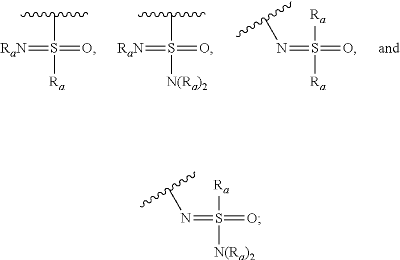
V is absent, C(Rₐ)₂, NRₐ, N(C═O)Rₐ, NSO₂Rₐ or O;
R₄ is selected from the group consisting of (C₁-C₆)alkyl, (C₃-C₇)cycloalkyl, (C₄-C₁₀)bicycloalkyl, (C₃-C₇)heterocycloalkyl, (C₄-C₁₀)heterobicycloalkyl, (C₄-C₁₀)heterospiroalkyl, aryl, and heteroaryl, each optionally substituted with one or more R₅;
or alternatively V and R₄ taken together form a (C₃-C₇)heterocycloalkyl or (C₄-C₁₀)heterospiroalkyl;
each occurrence of R₅ is independently selected from the group consisting of H, D, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₇)cycloalkyl, (C₄-C₁₀)bicycloalkyl, (C₃-C₇)heterocycloalkyl, halogenated (C₃-C₇)heterocycloalkyl, (C₄-C₁₀)heterobicycloalkyl, (C₄-C₁₀)heterospiroalkyl, aryl, heteroaryl, —ORₐ, —SRₐ, —N(Rₐ)₂, —CORₐ, —CO₂Rₐ, CON(Rₐ)₂, —CN, —NC, NO₂, N₃, —SO₂Rₐ, —SO₂N(Rₐ)₂, —N(Rₐ)SO₂Rₐ, N(Rₐ)CORₐ, 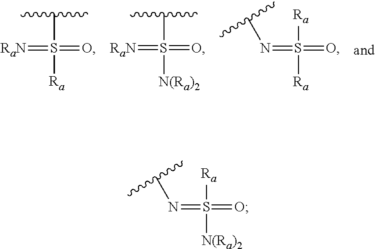
and
each occurrence of Rₐ is independently H, (C₁-C₆)alkyl, (C₂-C₆)alkenyl, (C₃-C₇)cycloalkyl, aryl, or heteroaryl, or two Rₐ taken together form a 4-6-membered ring optionally substituted with halogen or (C₁-C₆)alkyl.

2. The compound of claim 1, wherein Q, T, and U are each independently O, NH, NCH₃, N(C═O)H, N(C═O)CH₃, N(C═O)CH₂CH₃, NSO₂CH₃, or NSO₂CH₂CH₃, where valence permits.

3. The compound of claim 1, wherein X₁, X₂, X₃, X₄, X₅, X₆, X₇, X₈, X₉, Y₁, Y₂, Y₃, Y₄, Y₅, Z₁, Z₂, Z₃, Z₄, and Z₅ are each independently CH, C(halogen), or N.

4. The compound of claim 1, wherein \textcircled{A} is

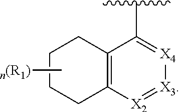

5. The compound of claim 4, wherein the structural moiety 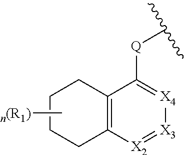
has the structure of

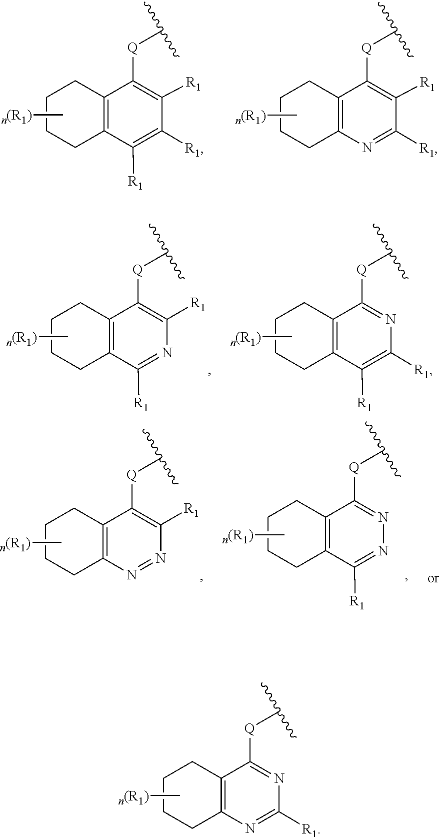

6. The compound of claim 4, wherein n is 0, 1, or 2.

7. The compound of claim 4, wherein the structural moiety 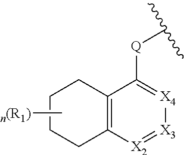
has the structure of

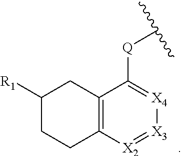

8. The compound of claim 7, wherein the structural moiety 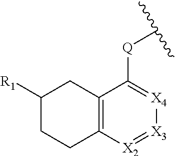
has the structure of

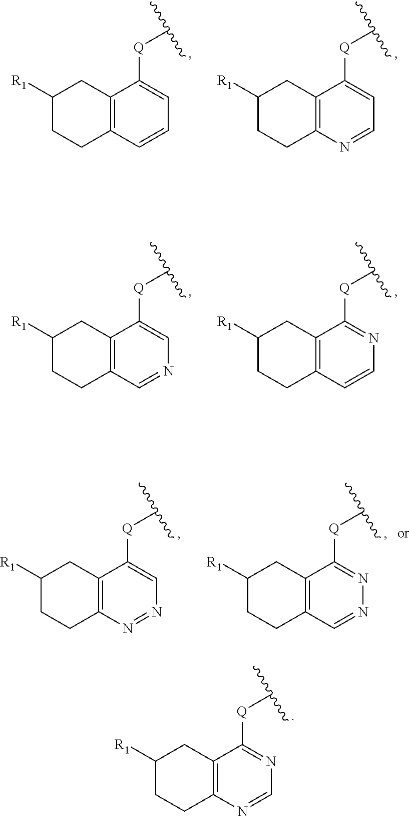

9. The compound of claim 1, wherein \textcircled{A} is

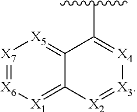

10. The compound of claim 9, wherein the structural moiety 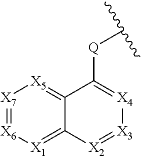
has the structure of

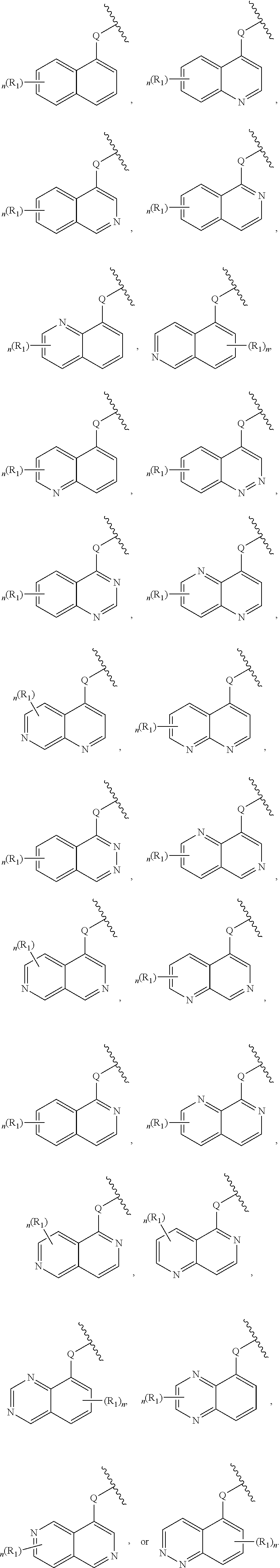

11. The compound of claim 10, wherein n is 0, 1, or 2.

12. The compound of claim 9, wherein the structural moiety 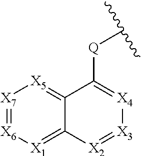
has the structure of

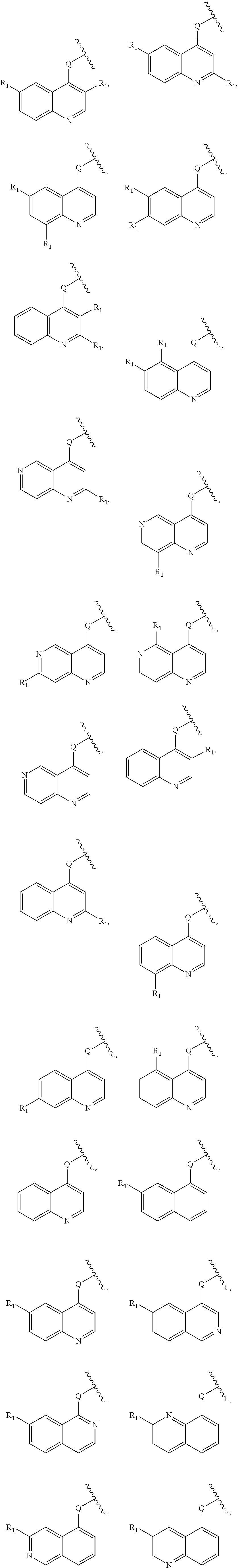

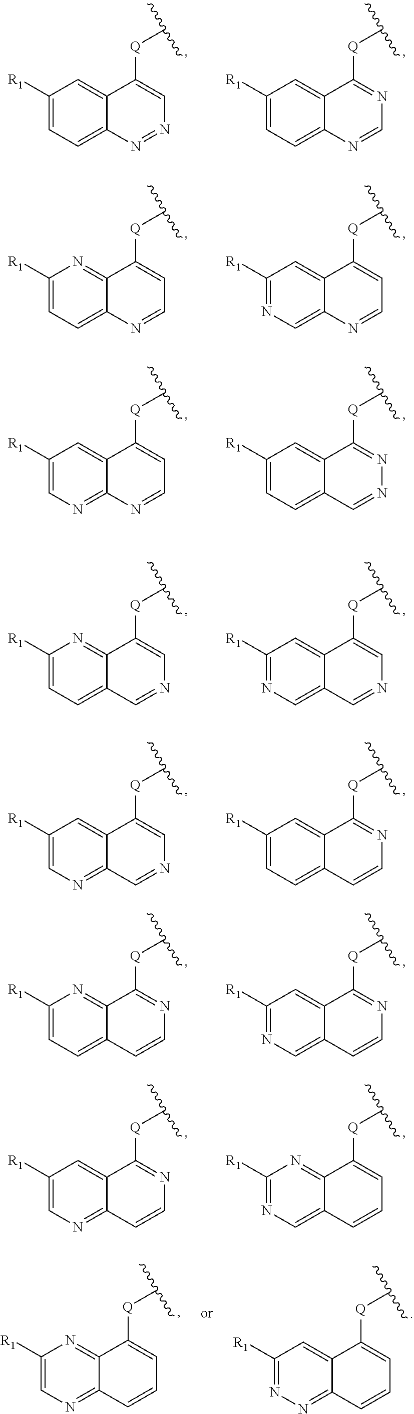

13. The compound of claim 9, wherein the structural moiety 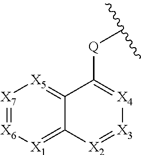
has the structure of

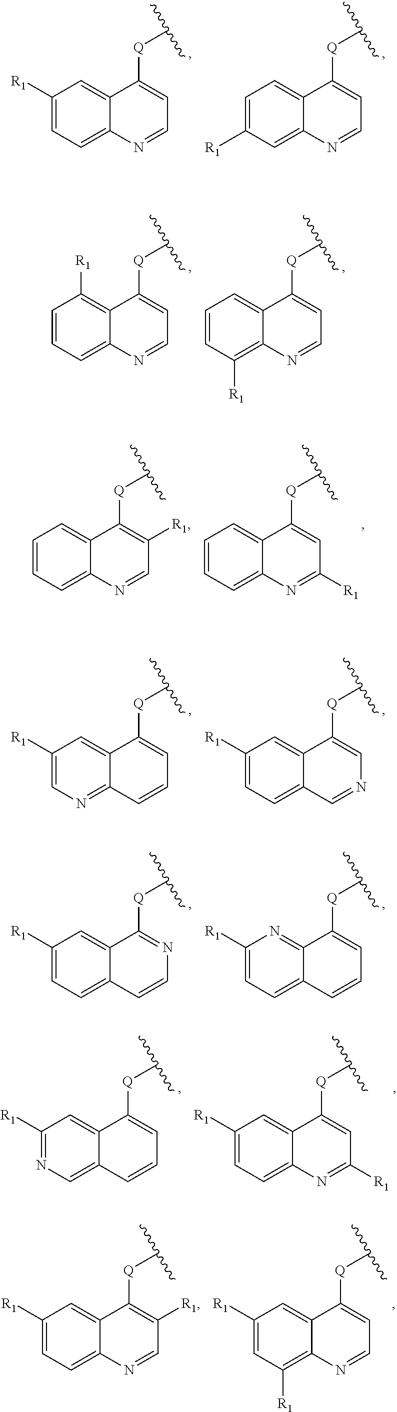

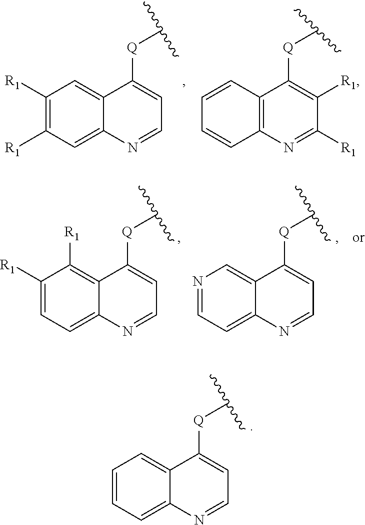

14. The compound of claim 9, wherein the structural moiety 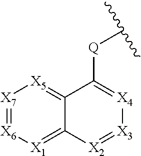
has the structure of

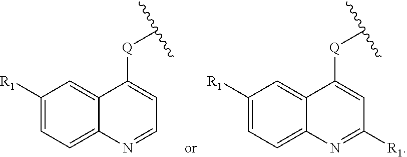

15. The compound of claim 1, wherein \textcircled{A} is

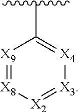

16. The compound of claim 15, wherein the structural moiety 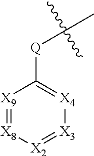
has the structure of

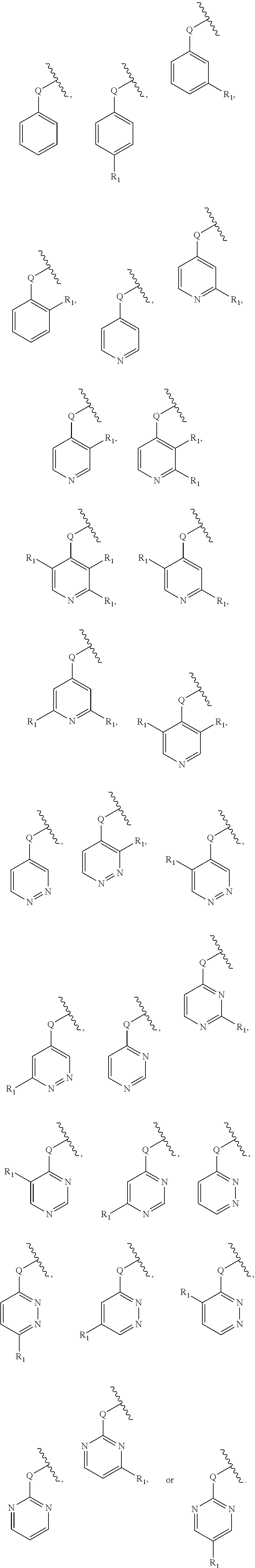

17. The compound of claim 15, wherein the structural moiety 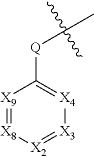
has the structure of

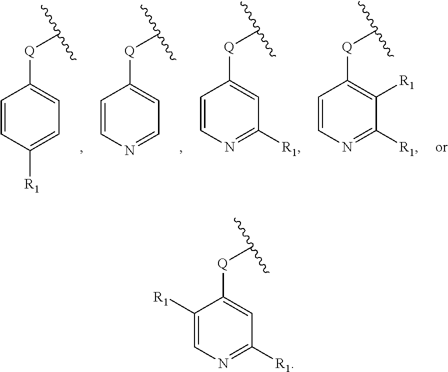

18. The compound of claim 1, wherein Q is O.

19. The compound of claim 1, wherein Q is NRₐ, N(C═O)Rₐ, or NSO₂Rₐ.

20. The compound of claim 1, wherein each occurrence of R₁ is independently H, D, halogen, ORₐ, N(Rₐ)₂, (C₁-C₆)alkyl, (C₁-C₆)alkynyl, (C₃-C₇)heterocycloalkyl, (C₄-C₁₀)heterospiroalkyl, halogenated (C₃-C₇)heterocycloalkyl, aryl, (C₄-C₁₀)bicycloalkyl, —CN, —NC, N₃, NO₂, CORₐ, CO₂Rₐ, CON(Rₐ)₂, —SO₂Rₐ, or —SO₂N(Rₐ)₂; wherein the (C₃-C₇)heterocycloalkyl is optionally substituted with one or more (C₁-C₆)alkyl.

21. The compound of claim 1, wherein each occurrence of R₁ is independently H, halogen, (C₁-C₆)alkyl, (C₃-C₇)heterocycloalkyl, (C₄-C₁₀)heterospiroalkyl, halogenated (C₃-C₇)heterocycloalkyl, N(Rₐ)₂, or —CN; wherein the (C₃-C₇)heterocycloalkyl is optionally substituted with one or more (C₁-C₆)alkyl.

22. The compound of claim 1, wherein each occurrence of R₁ is independently H, halogen, (C₁-C₆)alkyl, (C₁-C₆)alkyl, halogenated (C₃-C₇)heterocycloalkyl, or (C₃-C₇)heterocycloalkyl; wherein the (C₃-C₇)heterocycloalkyl is optionally substituted with one or more (C₁-C₆)alkyl.

23. The compound of claim 1, wherein each occurrence of R₁ is independently H, D, F, Cl, Br, CH₃, OCH₃, NH₂, NHCH₃, N(CH₃)₂, 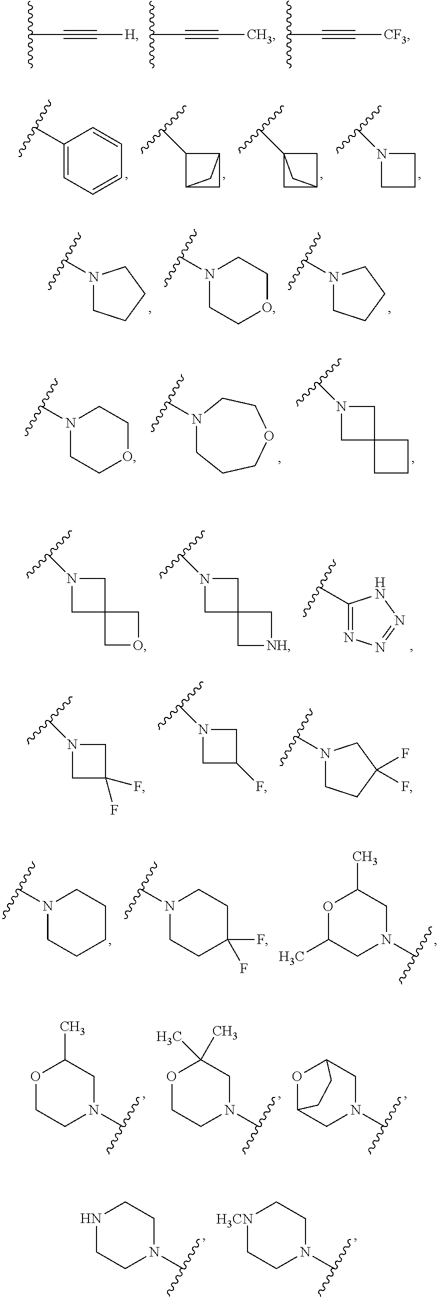
—CN, —NC, N₃, NO₂,

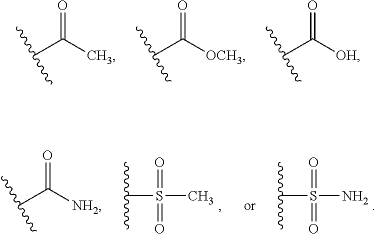

24. The compound of claim 1, wherein each occurrence of R₁ is independently H, D, F, CH₃, NH₂, NHCH₃, N(CH₃)₂,

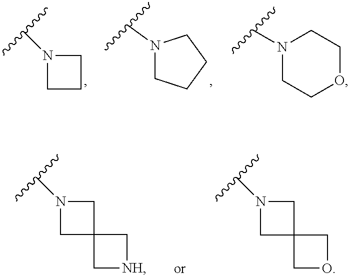 [SMILES: *N1CCC1 ; *N1CCCC1 ; *N1CCOCC1 ; *N1CC2(CNC2)C1 ; *N1CC2(COC2)C1]

25. The compound of claim 1, wherein at least one occurrence of R₁ is

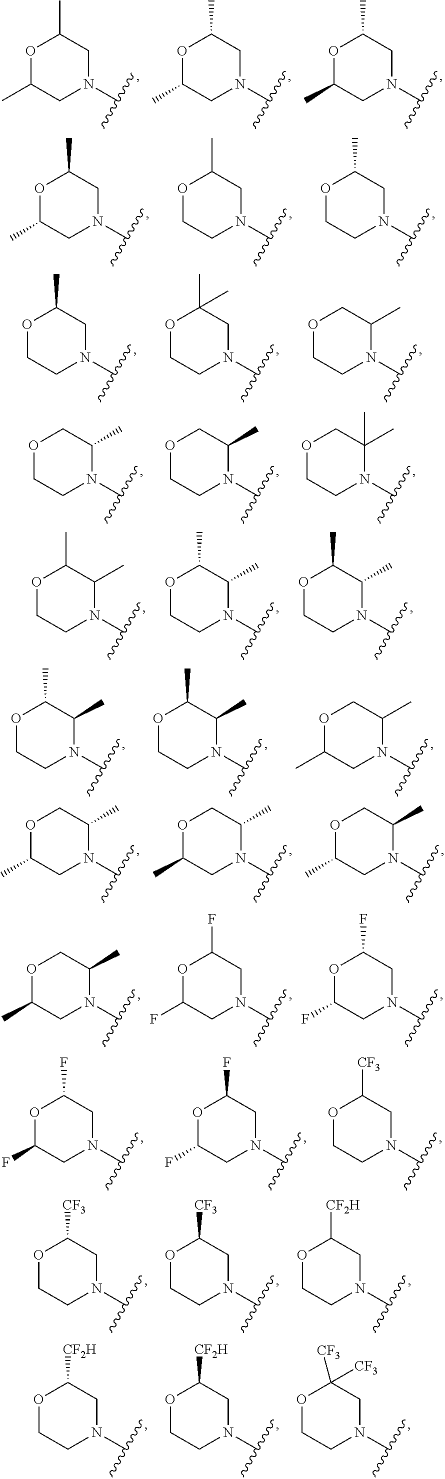

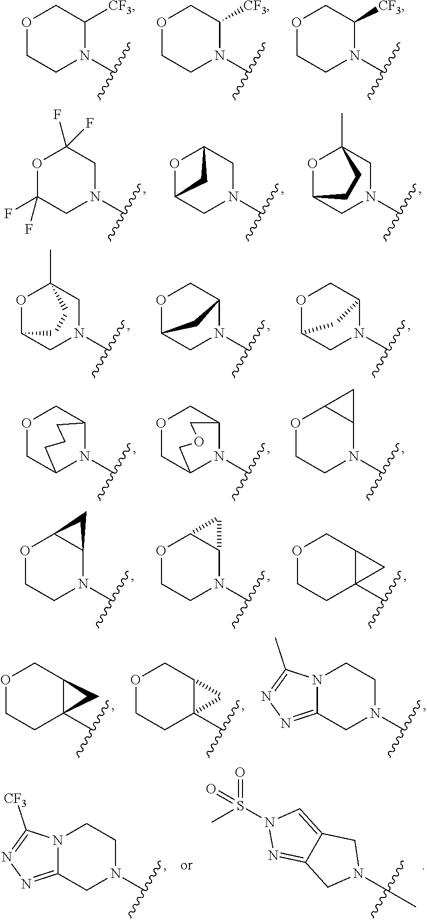

26. The compound of claim 1, wherein at least one occurrence of R₁ is 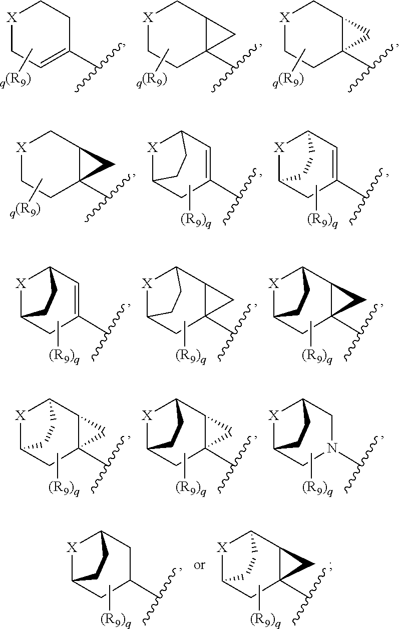
wherein X is CR₁₅, O, NR₁₄, or S; each occurrence of R₉ is independently H, (C₁-C₆)alkyl, halogenated (C₁-C₆)alkyl, halogen, —ORₐ, —CN, or —N(Rₐ)₂; R₁₄ is H, (C₁-C₆)alkyl, (C₃-C₇)cycloalkyl, (C₃-C₇)heterocycloalkyl, aryl, or heteroaryl; each occurrence of R₁₅ is independently H, (C₁-C₆)alkyl, halogenated (C₁-C₆)alkyl, halogen, —ORₐ, —CN, or —N(Rₐ)₂; and q is 0, 1, 2, or 3.

27. The compound of claim 26, wherein X is O.

28. The compound of claim 26, wherein each occurrence of R₉ is independently H, (C₁-C₆)alkyl, halogenated (C₁-C₆)alkyl, or halogen.

29. The compound of claim 26, wherein each occurrence of R₉ is independently H, F, Cl, Br, CH₃, CF₃, OH, NH₂, —NHCH₃, or —N(CH₃)₂.

30. The compound of claim 26, wherein each occurrence of R₉ is independently H, F, Cl, Br or CH₃.

31. The compound of claim 26, wherein each occurrence of R₁₅ is independently H, (C₁-C₆)alkyl, halogenated (C₁-C₆)alkyl, or halogen.

32. The compound of claim 26, wherein each occurrence of R₁₅ is independently H, F, Cl, Br or CH₃.

33. The compound of claim 26, wherein q is 0.

34. The compound of claim 26, wherein q is 1.

35. The compound of claim 26, wherein q is 2 or 3.

36. The compound of claim 1, wherein X is NR₁₄ and R₁₄ is H or (C₁-C₆)alkyl.

37. The compound of claim 1, wherein at least one occurrence of R₁ is 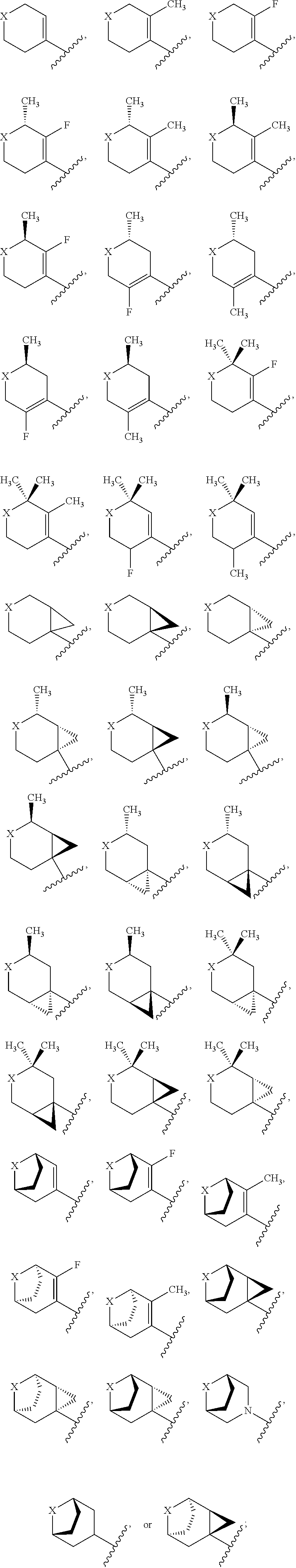
wherein X is O or NR₁₄; and R₁₄ is H or (C₁-C₆)alkyl.

38. The compound of claim 1, wherein at least one occurrence of R₁ is

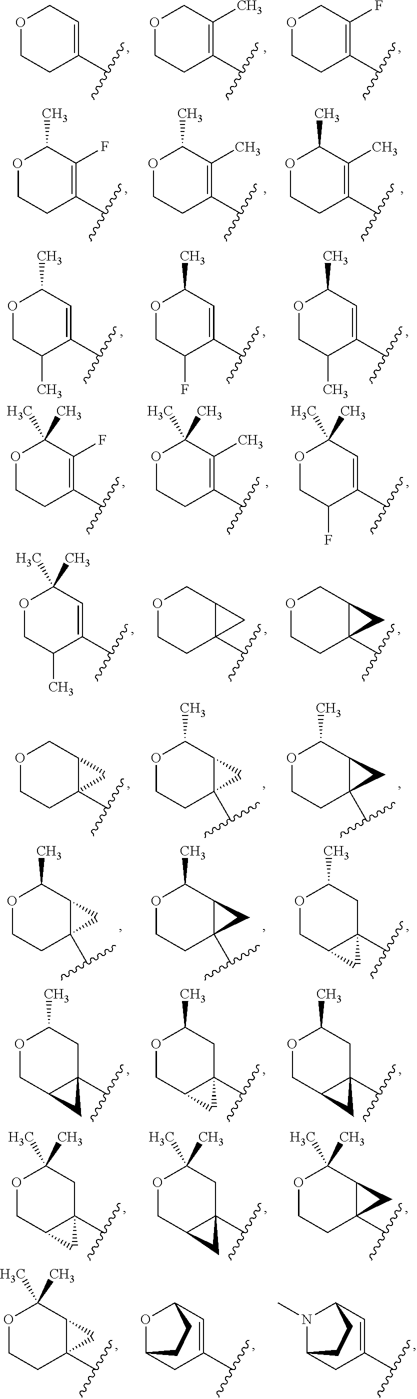

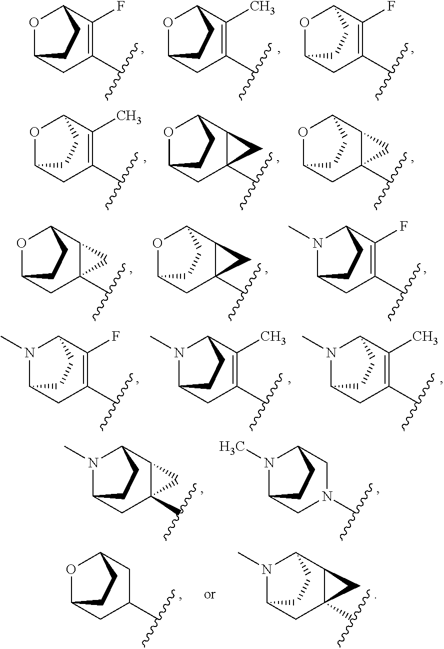

39. The compound of claim 1, wherein \textcircled{A} is 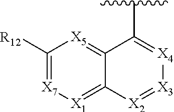
wherein R₁₂ is (C₃-C₇)cycloalkenyl, (C₃-C₇)heterocycloalkenyl, (C₄-C₁₀)bicycloalkenyl, (C₄-C₁₀)heterobicycloalkenyl, (C₄-C₁₄)tricycloalkenyl, or (C₄-C₁₄)heterotricycloalkenyl, each of which is optionally substituted by one or more (C₁-C₆)alkyl, halogenated (C₁-C₆)alkyl, halogen, —ORₐ, —CN, or —N(Rₐ)₂.

40. The compound of claim 39, wherein R₁₂ is 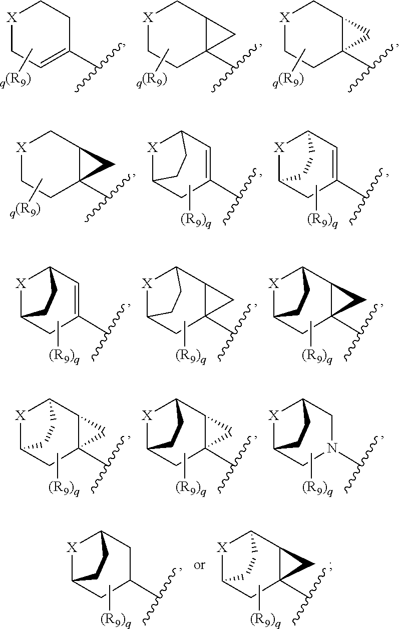
wherein X is CR₁₅, O, NR₁₄, or S; each occurrence of R₉ is independently H, (C₁-C₆)alkyl, halogenated (C₁-C₆)alkyl, halogen, —ORₐ, —CN, or —N(Rₐ)₂; R₁₄ is H, (C₁-C₆)alkyl, (C₃-C₇)cycloalkyl, (C₃-C₇)heterocycloalkyl, aryl, or heteroaryl; each occurrence of R₁₅ is independently H, (C₁-C₆)alkyl, halogenated (C₁-C₆)alkyl, halogen, —ORₐ, —CN, or —N(Rₐ)₂; and q is 0, 1, 2, or 3.

41. The compound of claim 39, wherein R₁₂ is 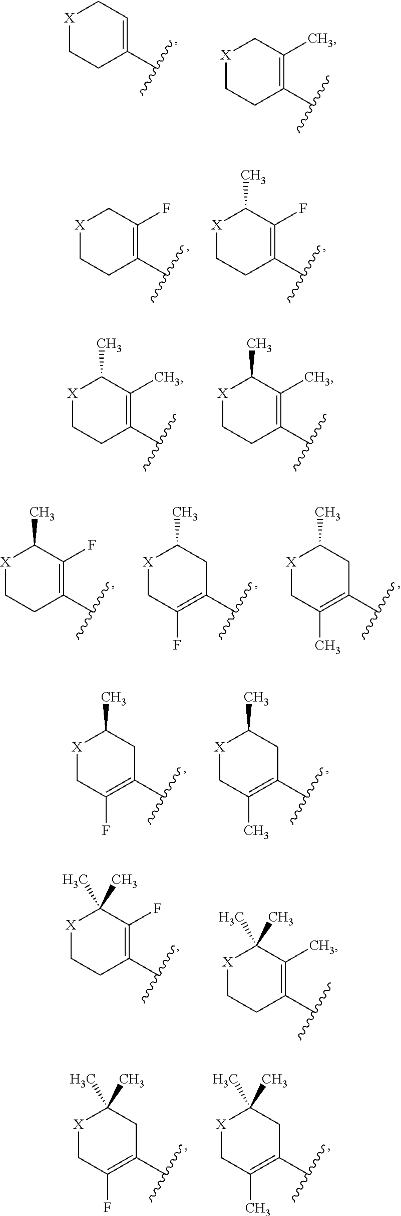 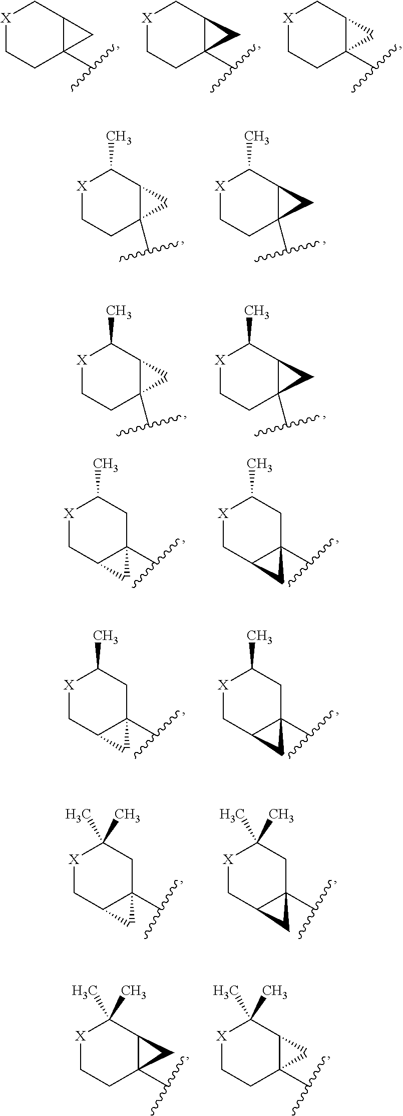 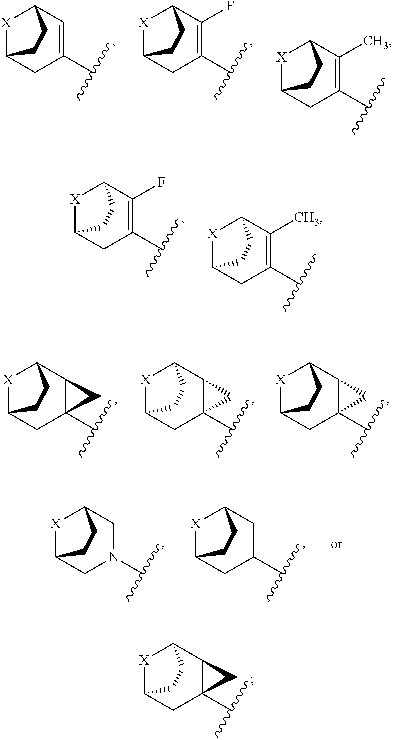
wherein X is O or NR₁₄; and R₁₄ is H or (C₁-C₆)alkyl.

42. The compound of claim 39, wherein R₁₂ is

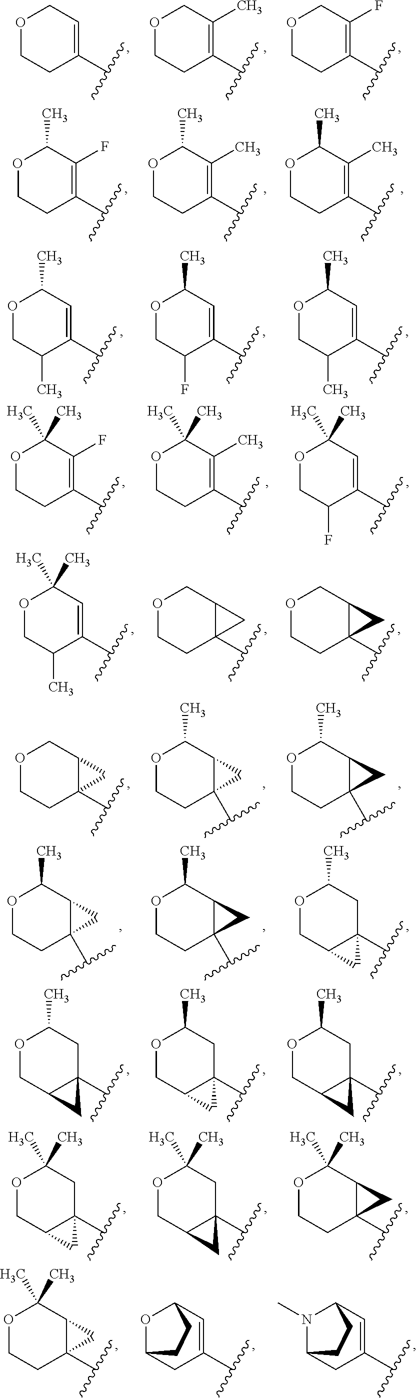

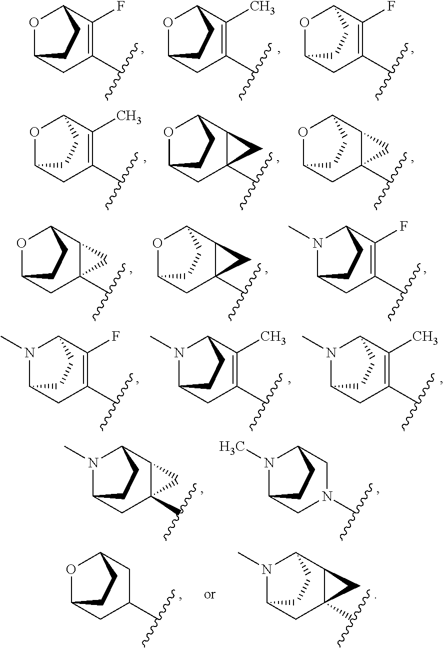

43. The compound of claim 25, wherein \textcircled{A} is

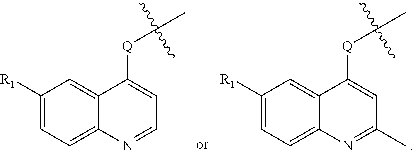

44. The compound of claim 1, wherein the structural moiety 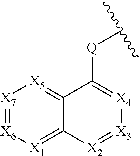
has the structure of 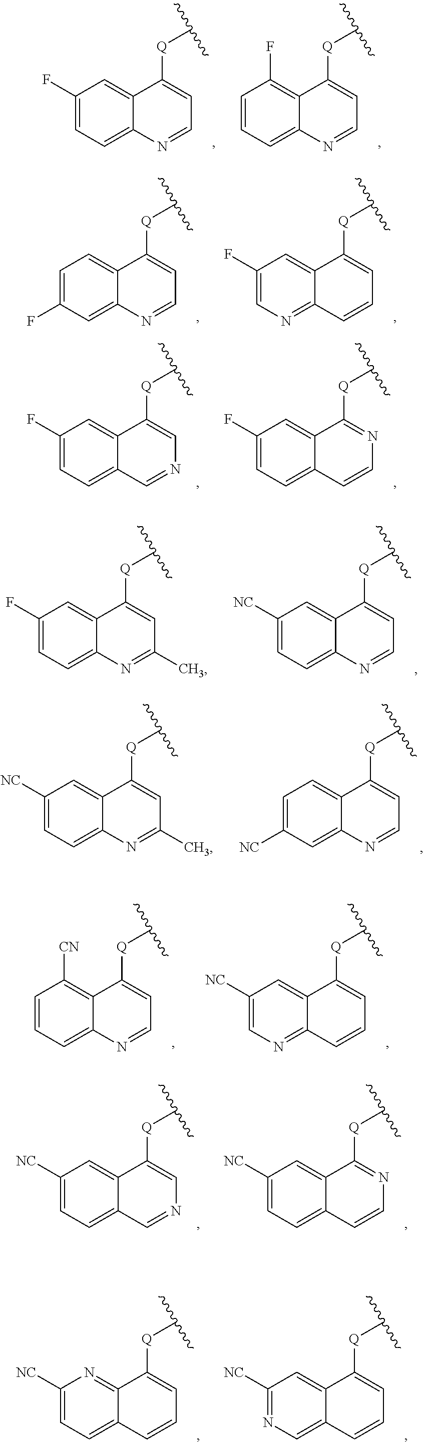 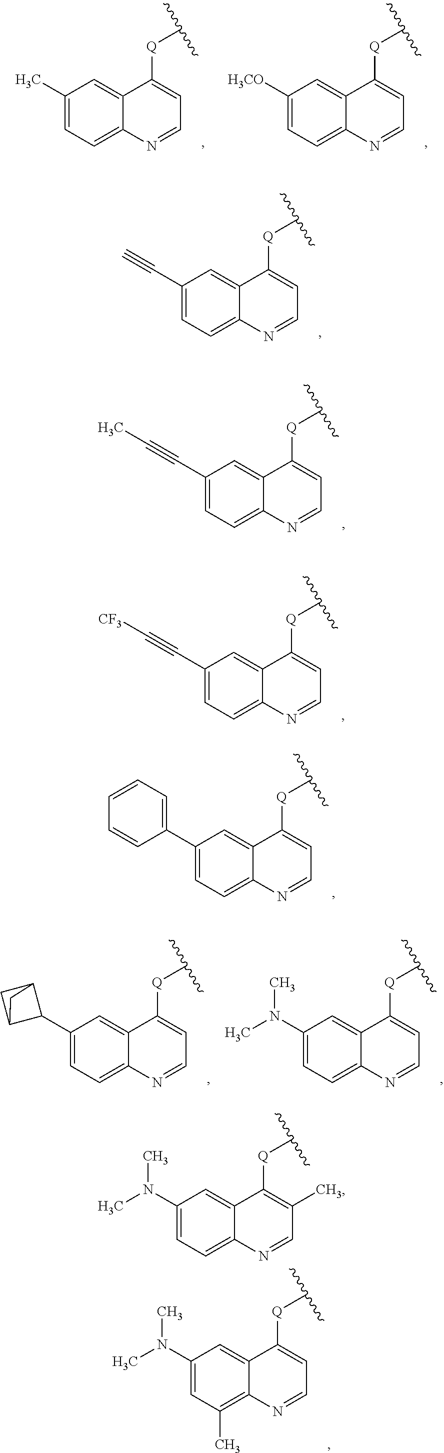 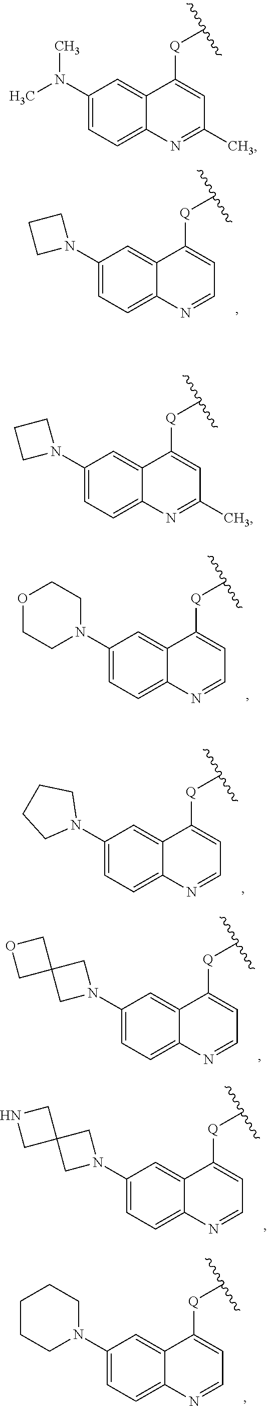 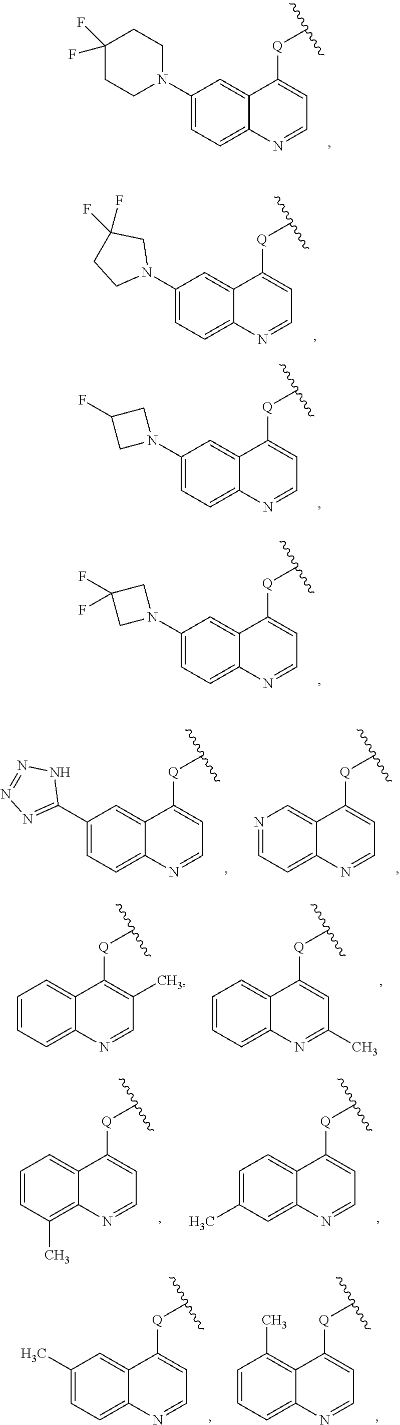 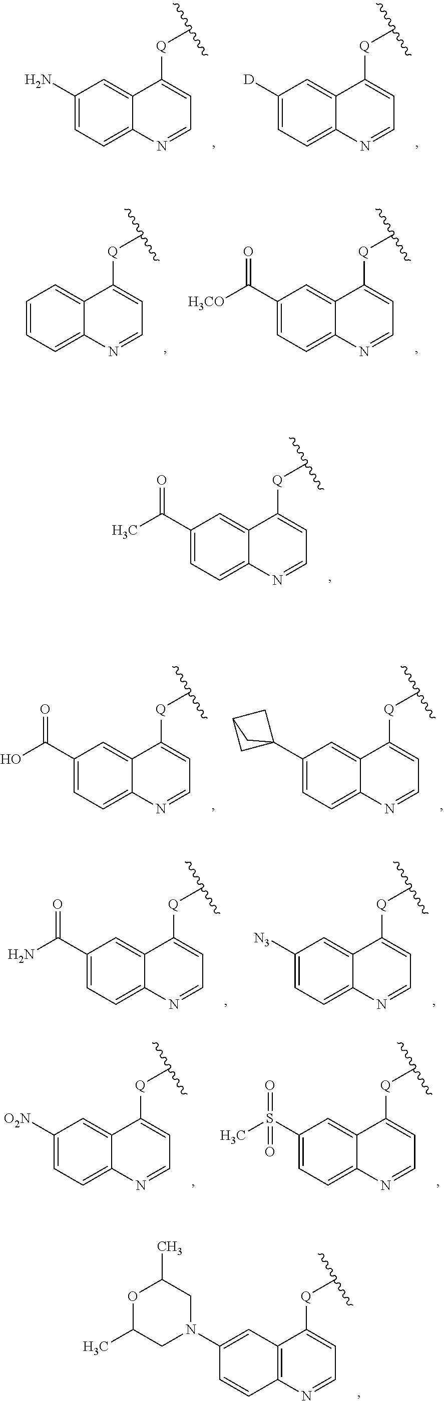 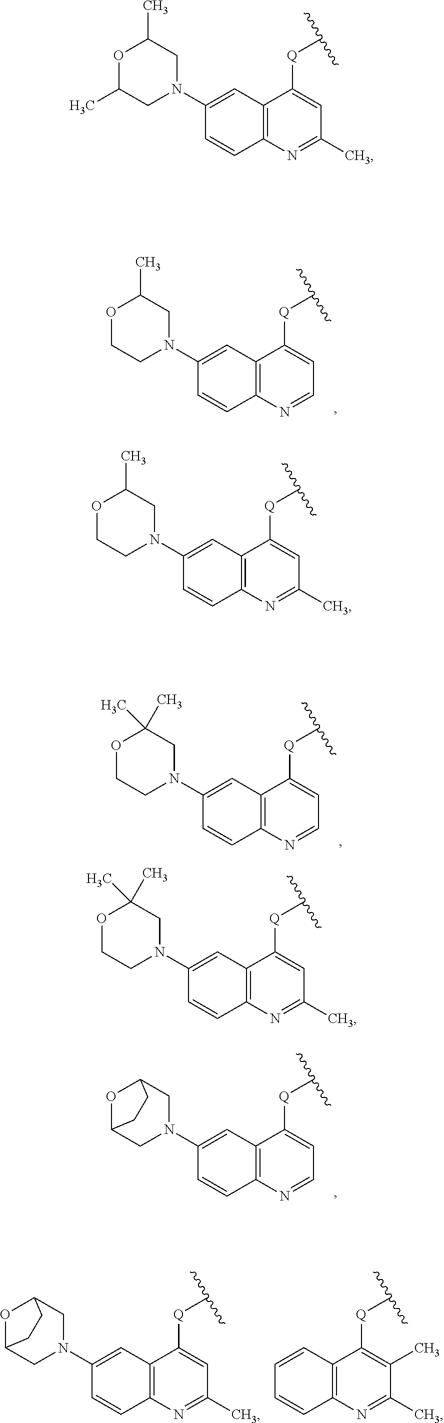 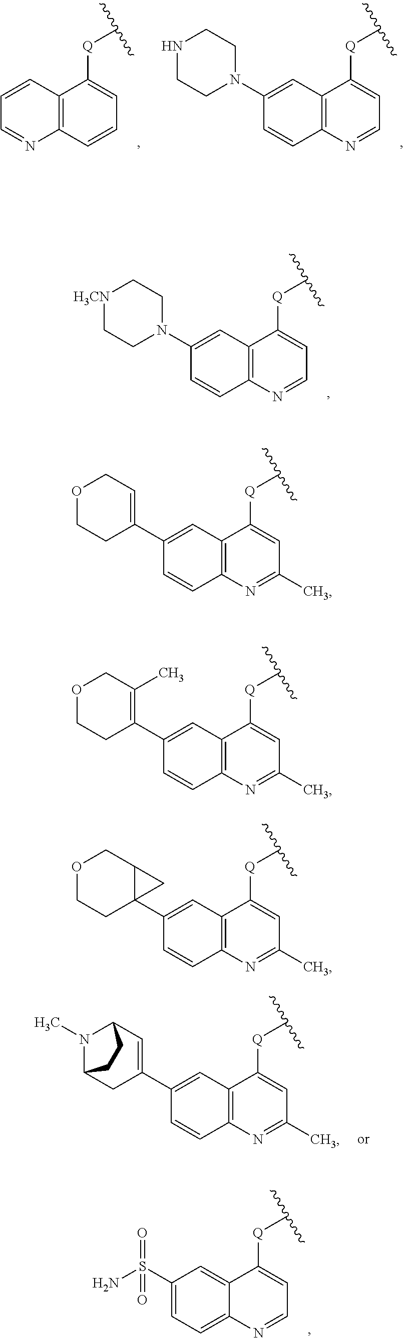
wherein Q is O or NH.

45. The compound of claim 1, wherein the structural moiety 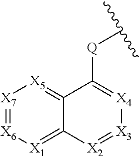
has the structure of 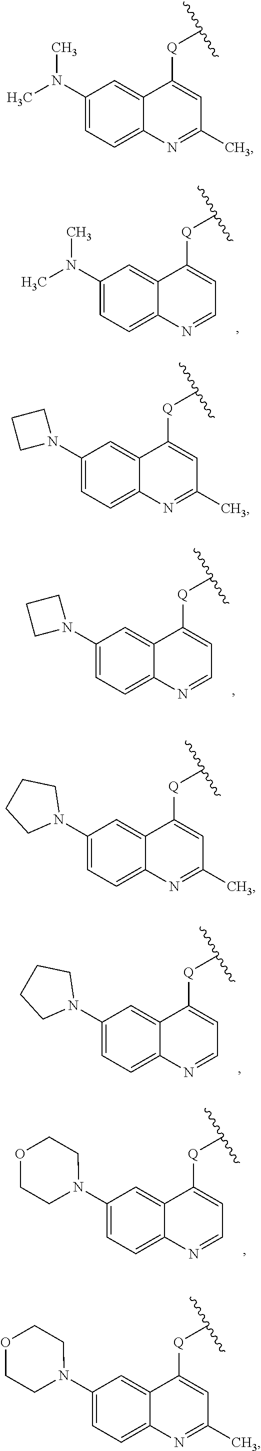 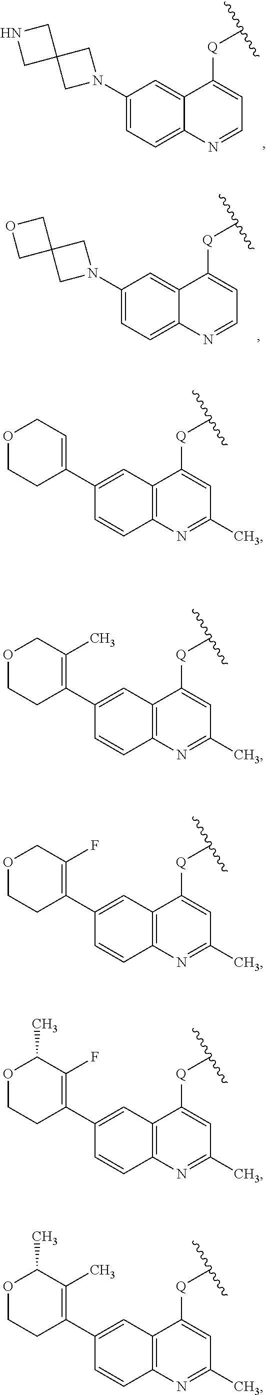 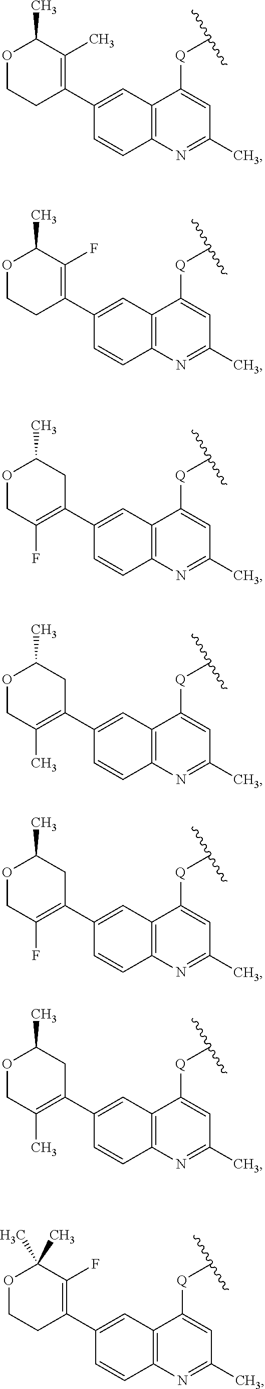 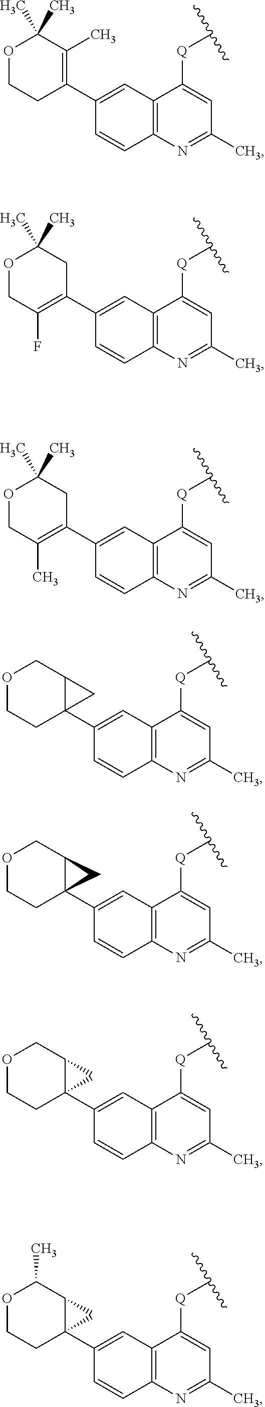 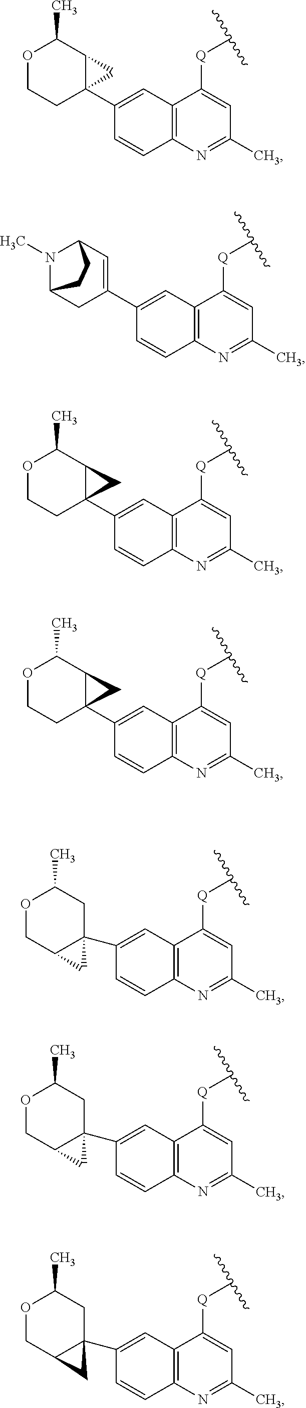 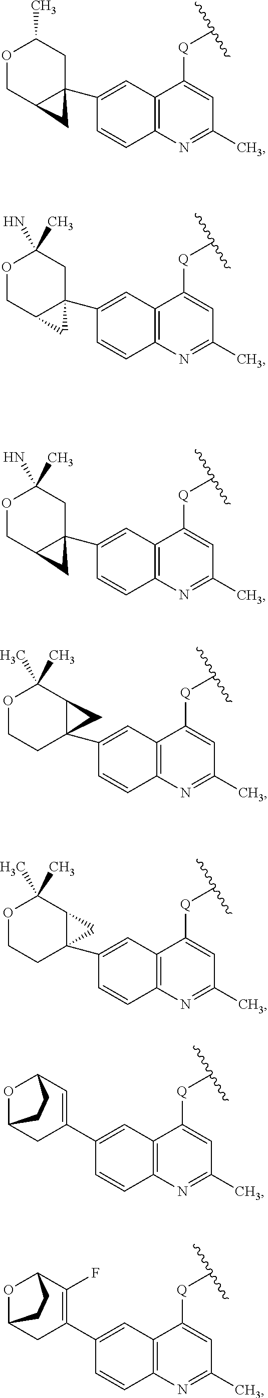 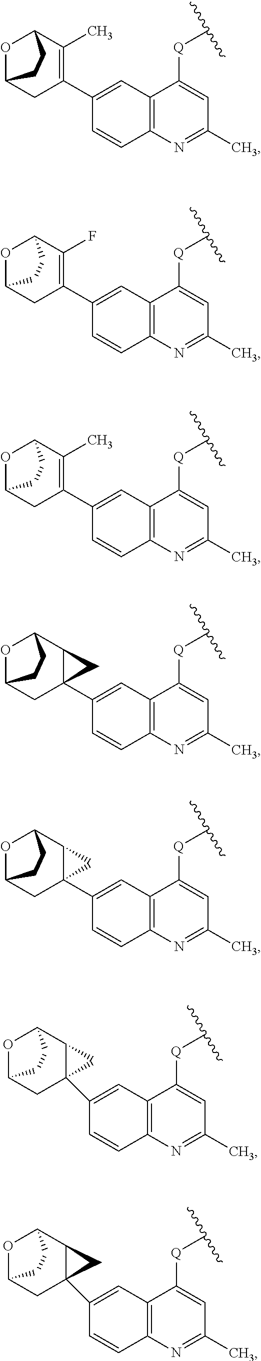 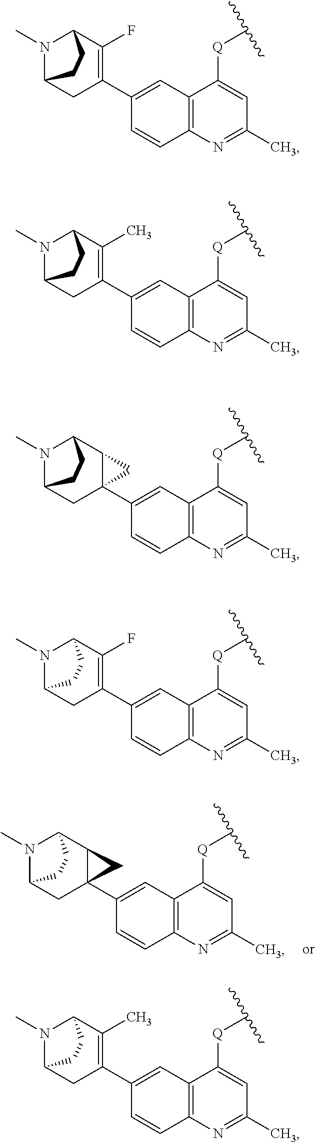
wherein Q is O or NH.

46. The compound of claim 1, wherein the structural moiety 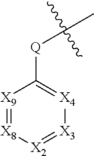
has the structure of 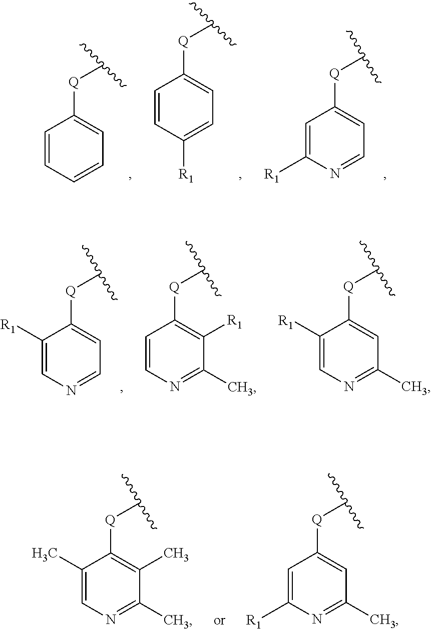
wherein Q is O or NH and R₁ is H, (C₁-C₆)alkyl, (C₃-C₇)heterocycloalkyl, halogenated (C₃-C₇)heterocycloalkyl, or halogen.

47. The compound of claim 1, wherein the structural moiety 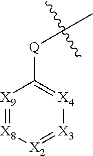
has the structure of 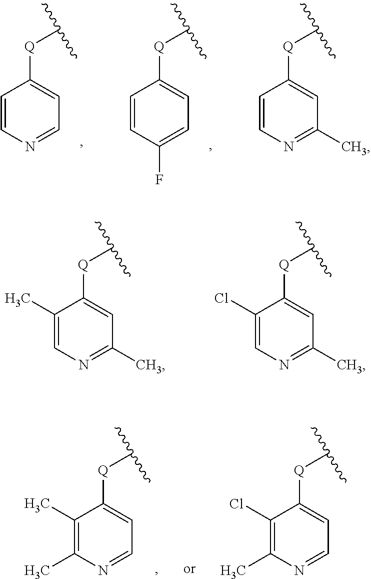
wherein Q is O or NH.

48. The compound of claim 1, wherein the structural moiety 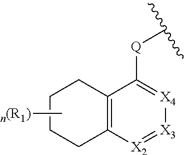
has the structure of 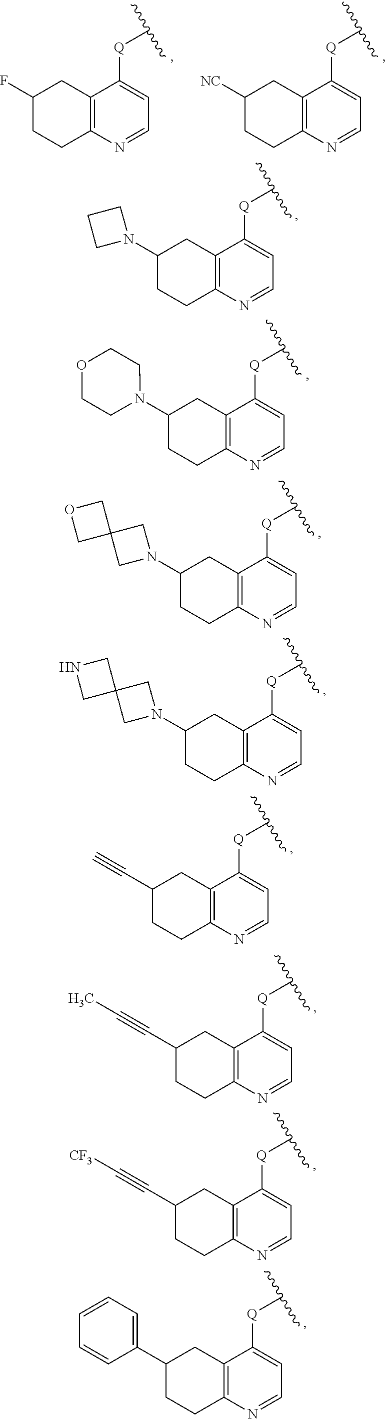 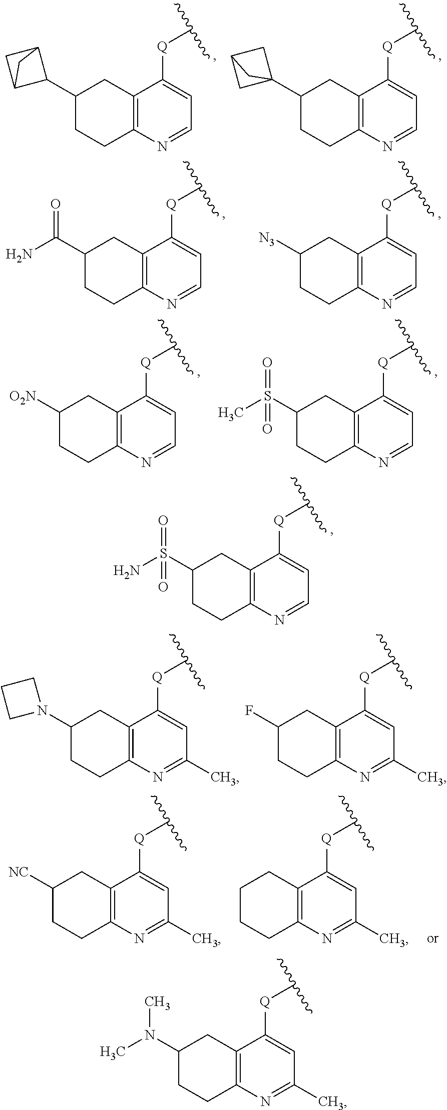
wherein Q is O or NH.

49. The compound of claim 1, having the formula of Formula Ia.

50. The compound of claim 1, wherein the structural moiety 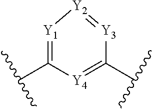
has the structure of

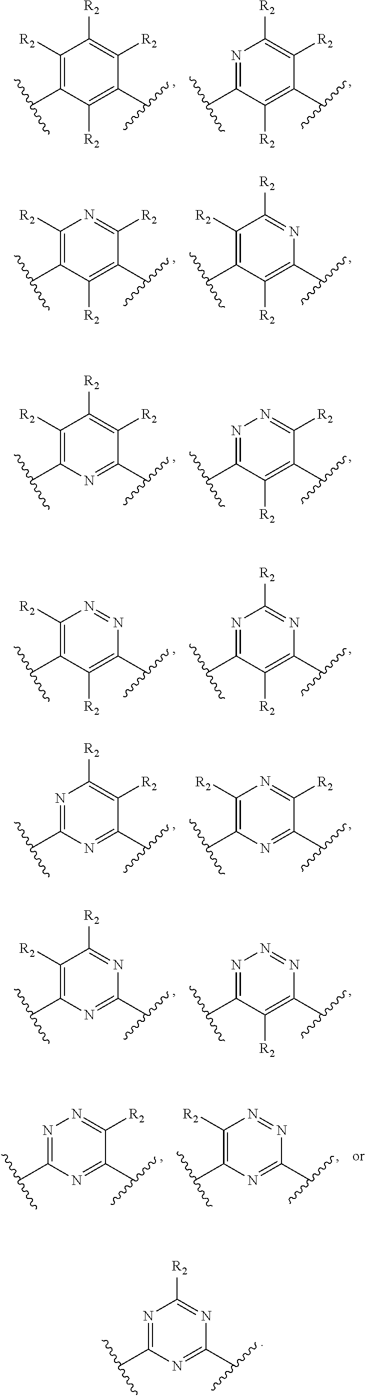

51. The compound of claim 1, wherein the structural moiety 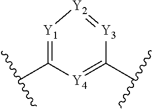
has the structure of

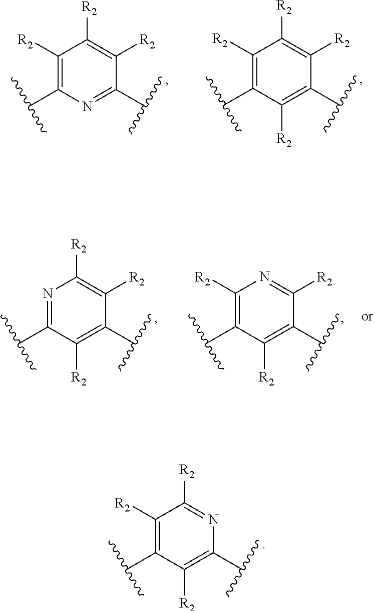

52. The compound of claim 1, wherein each occurrence of R₂ is independently H, halogen, CH₃, CF₃, OH, NH₂, —NHCH₃, or —N(CH₃)₂.

53. The compound of claim 1, wherein the structural moiety 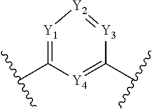
has the structure of

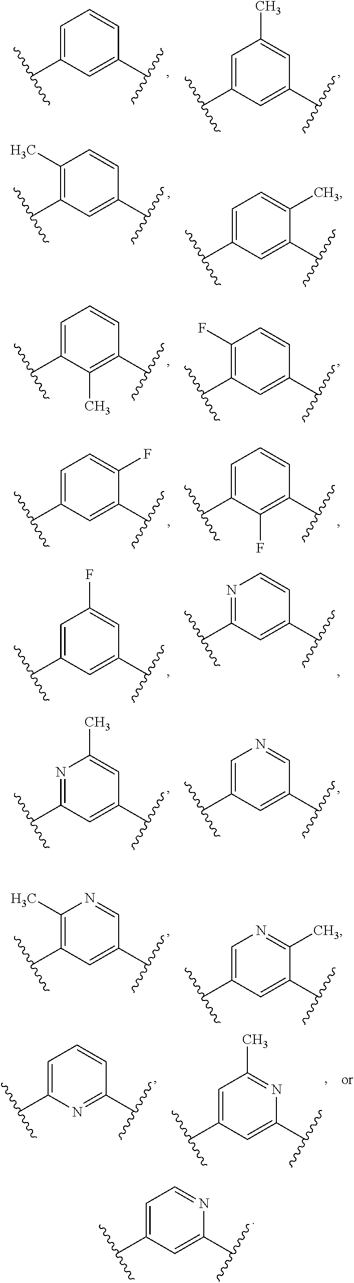

54. The compound of claim 1, wherein the structural moiety 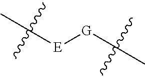
has the structure of

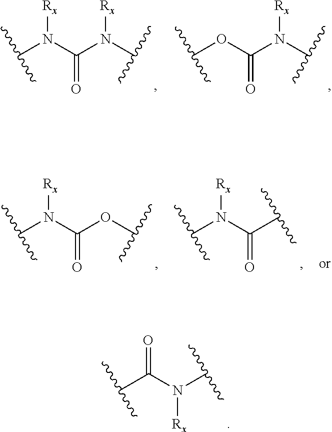

55. The compound of claim 1, wherein the structural moiety 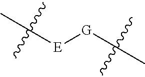
has the structure of

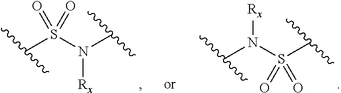

56. The compound of claim 1, wherein has the structure of 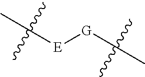
the structural moiety

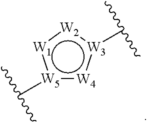

57. The compound of claim 1, wherein the structural moiety 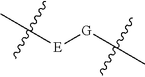
has the structure of

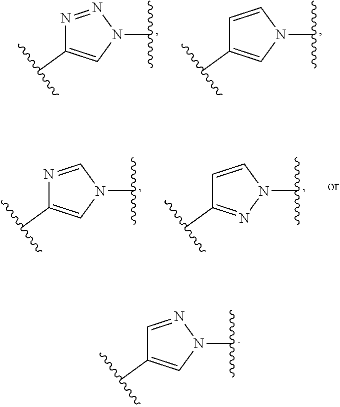 [SMILES: *c1cn(*)nn1 ; *c1ccn(*)c1 ; *c1cn(*)cn1 ; *c1ccn(*)n1 ; *c1cnn(*)c1]

58. The compound of claim 1, wherein the structural moiety 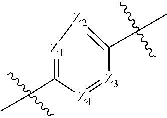
has the structure of

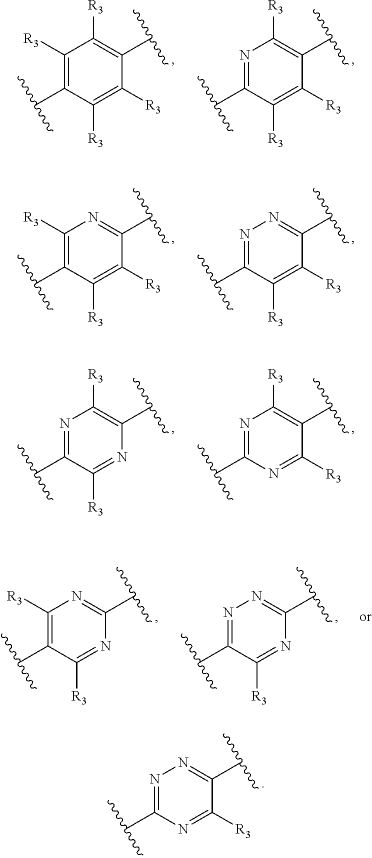

59. The compound of claim 1, wherein the structural moiety 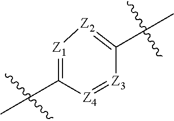
has the structure of

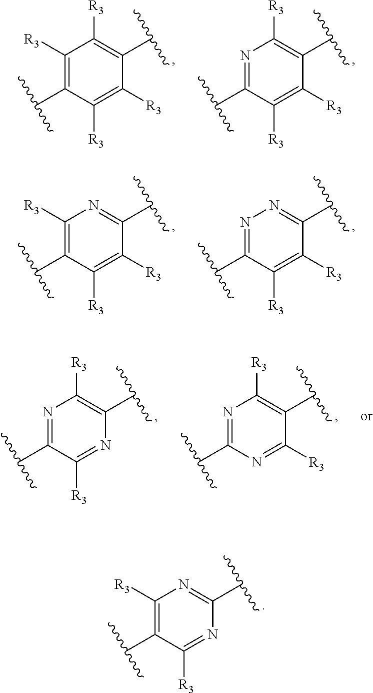

60. The compound of claim 1, wherein each occurrence of R₃ is H, halogen, CH₃, CF₃, OH, NH₂, —NHCH₃, or —N(CH₃)₂.

61. The compound of claim 1, wherein the structural moiety 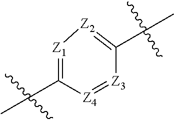
has the structure of

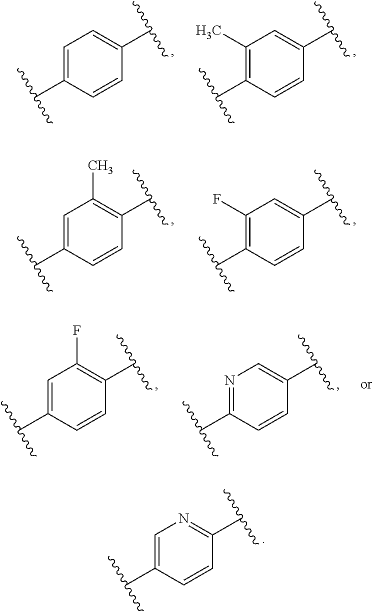

62. The compound of claim 1, wherein the structural moiety 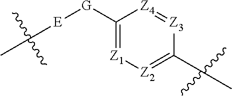
has the structure of 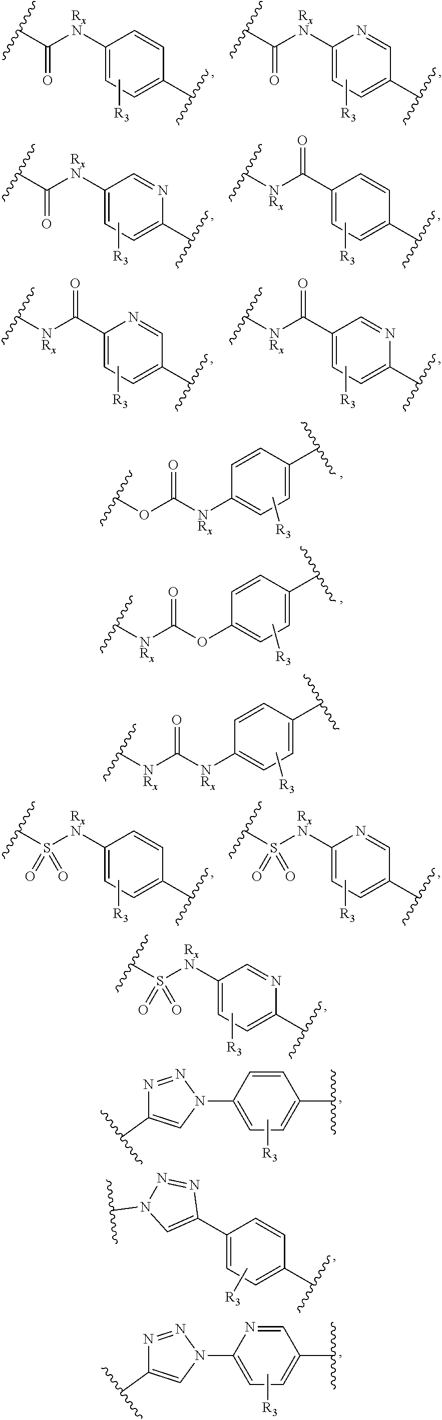 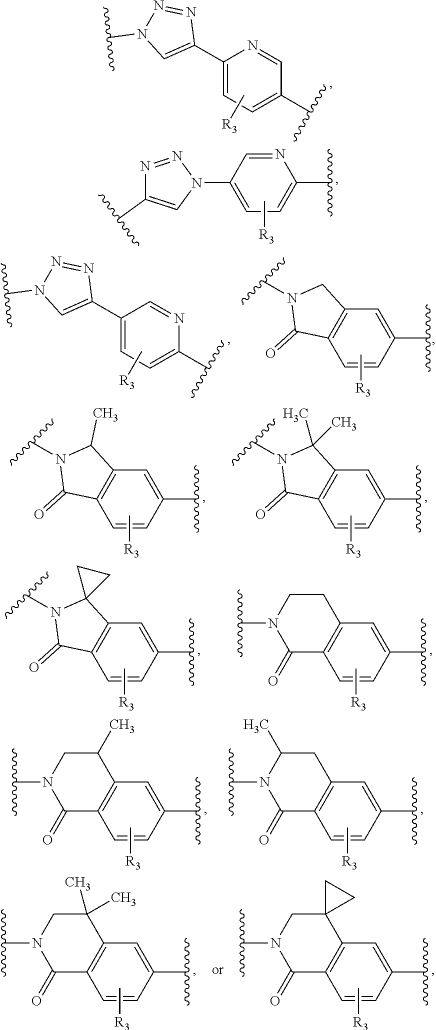
wherein R₃ is H, CH₃, OH, halogen, or NH₂; and wherein Rₓ is H, CH₃, or CH₂CH₃.

63. The compound of claim 1, wherein the structural moiety 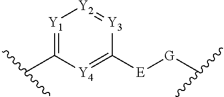
has the structure of 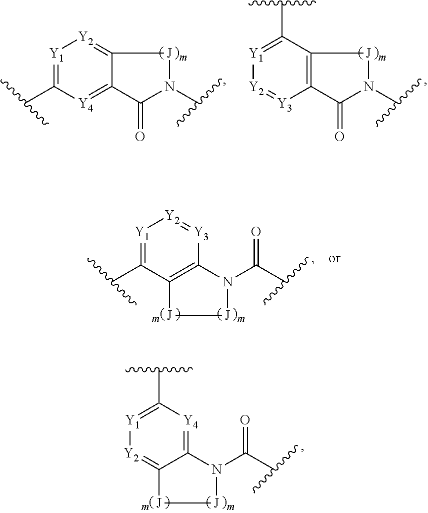
wherein each occurrence of m is independently 1 or 2, J is C(R_{y})₂, and each occurrence of R_{y} is independently H, (C₁-C₆)alkyl, OH, O(C₁-C₆)alkyl, or halogen; or any two R_{y} groups taken together with the carbon atom(s) they are connected to form a (C₃-C₇)cycloalkyl or (C₃-C₇)heterocycloalkyl.

64. The compound of claim 1, wherein the structural moiety 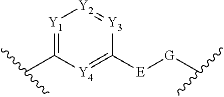
has the structure of 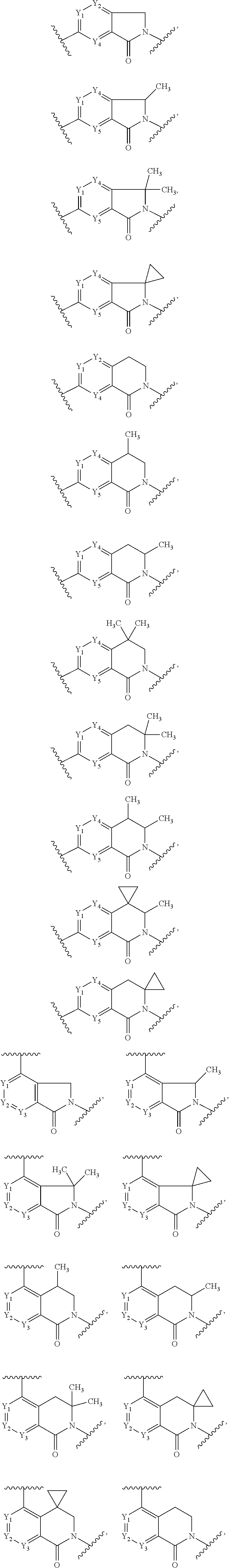 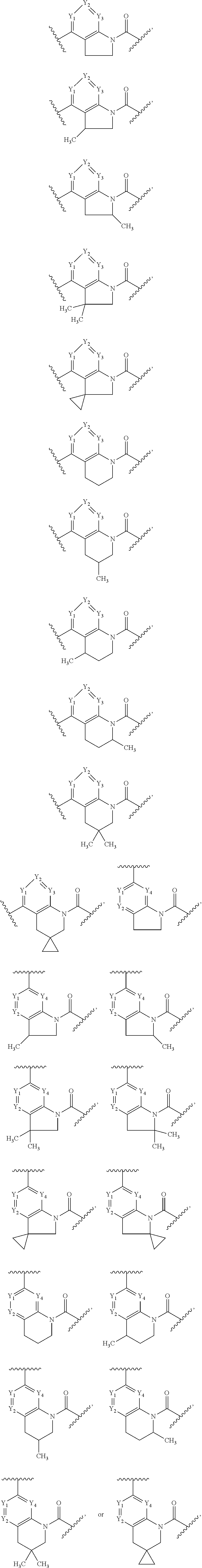
wherein Y₁, Y₂, Y₃, and Y₄ are each independently N, CH, CCH₃, or CF.

65. The compound of claim 1, wherein the structural moiety 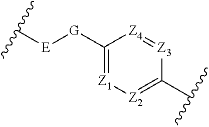
has the structure of 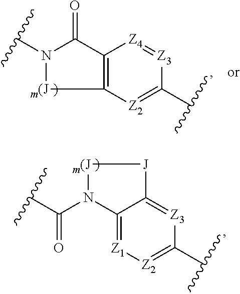
wherein each occurrence of m is independently 1 or 2, J is C(R_{z})₂, and each occurrence of R_{z} is independently H, (C₁-C₆)alkyl, OH, O(C₁-C₆)alkyl, or halogen; or any two R_{z} groups taken together with the carbon atom(s) they are connected to form a (C₃-C₇)cycloalkyl or (C₃-C₇)heterocycloalkyl.

66. The compound of claim 1, wherein the structural moiety 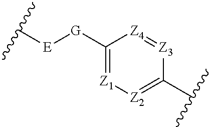
has the structure of 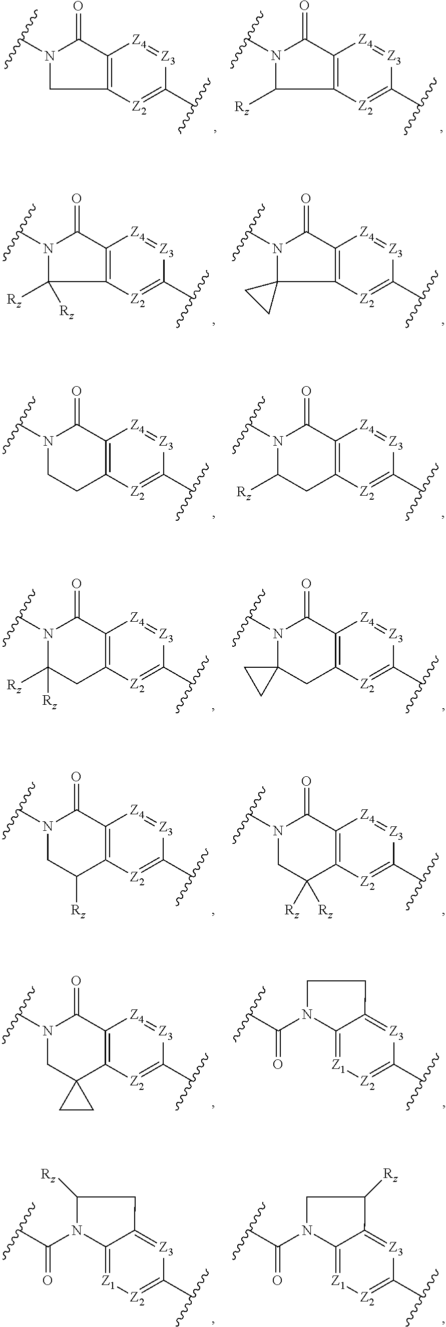 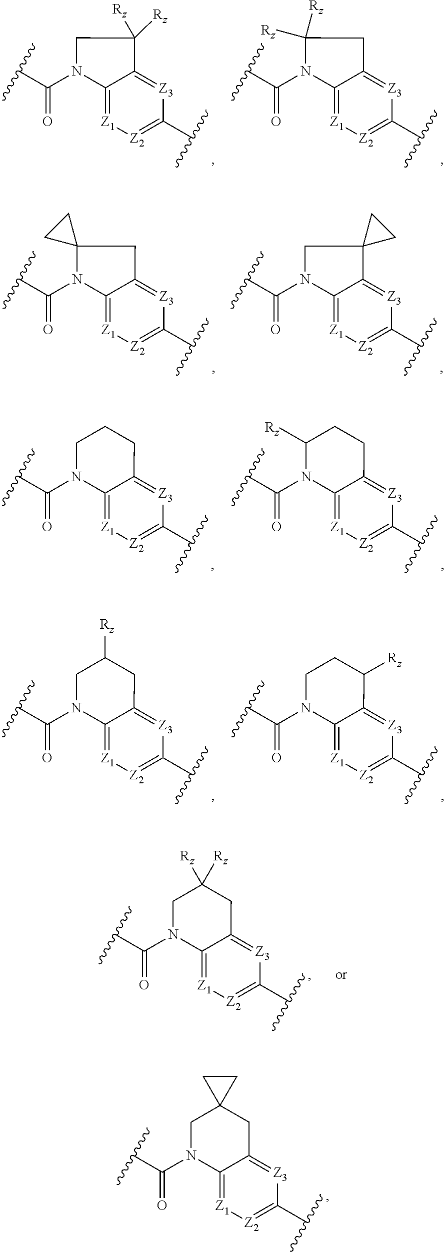
wherein Z₁, Z₂, Z₃, and Z₄ are each independently N, CH, CCH₃, or CF.

67. The compound of claim 1, wherein the structural moiety 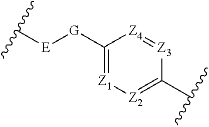
has the structure of

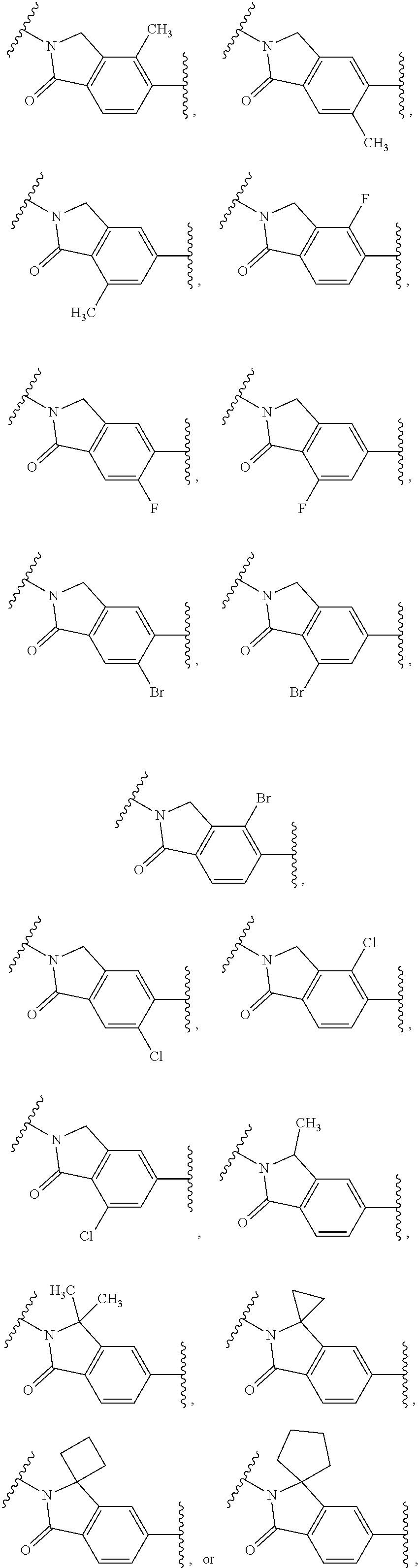

68. The compound of claim 1, having the formula of Formula Ib.

69. The compound of claim 1, wherein the structural moiety 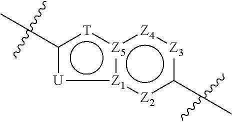
has the structure of 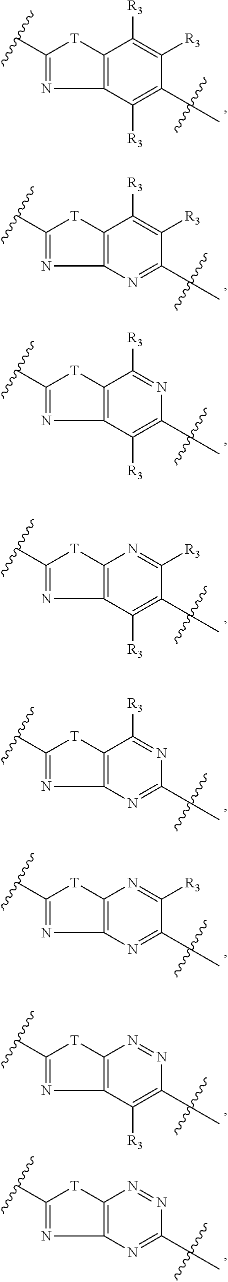 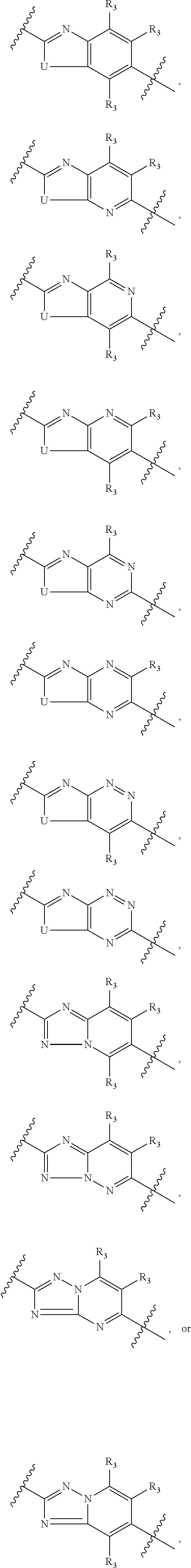
wherein each occurrence of T and U is independently O, N, NRₐ, N(C═O)Rₐ, NC(R_{b})₂OP(═O)(OR_{b})₂, or NSO₂Rₐ where valance permits.

70. The compound of claim 1, wherein the structural moiety 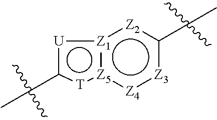
has the structure of 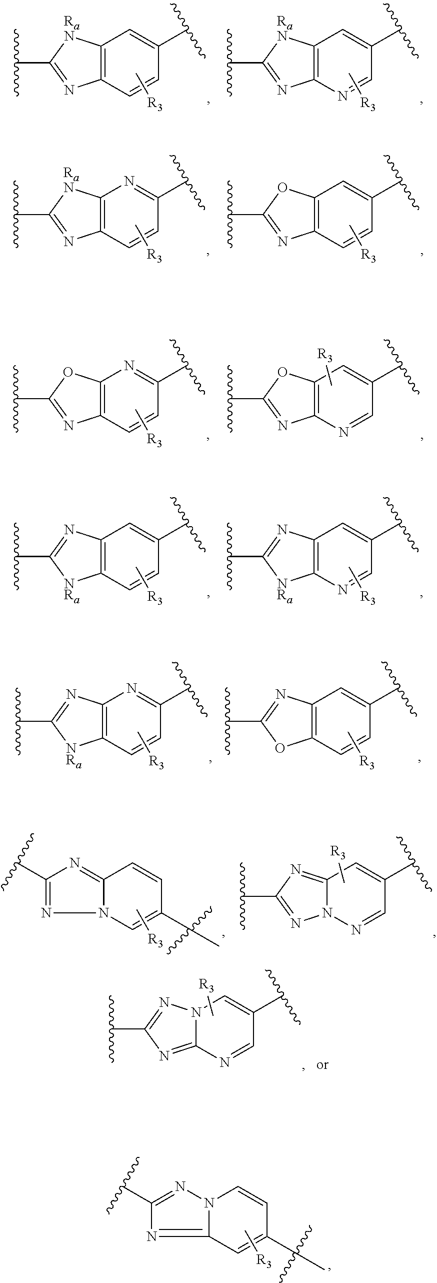
wherein R₃ is H, CH₃, OH, halogen, or NH₂; and wherein Rₐ is H, CH₃, or CH₂CH₃.

71. The compound of claim 1, wherein the structural moiety 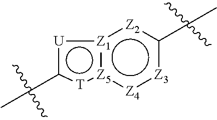
has the structure of

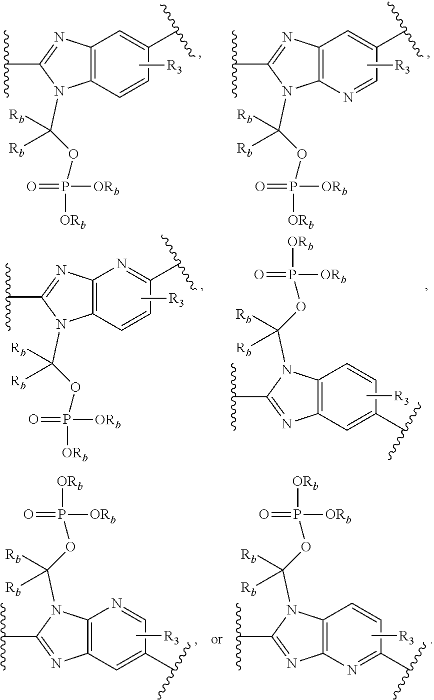

72. The compound of claim 69, wherein each occurrence of R_{b} is independently H or (C₁-C₆)alkyl.

73. The compound of claim 69, wherein each occurrence of R_{b} is independently H, CH₃, CH₂CH₃, or CH(CH₃)₂.

74. The compound of claim 1, having the formula of Formula Ic.

75. The compound of claim 1, wherein the structural moiety 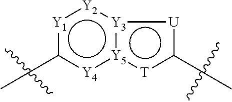
has the structure of 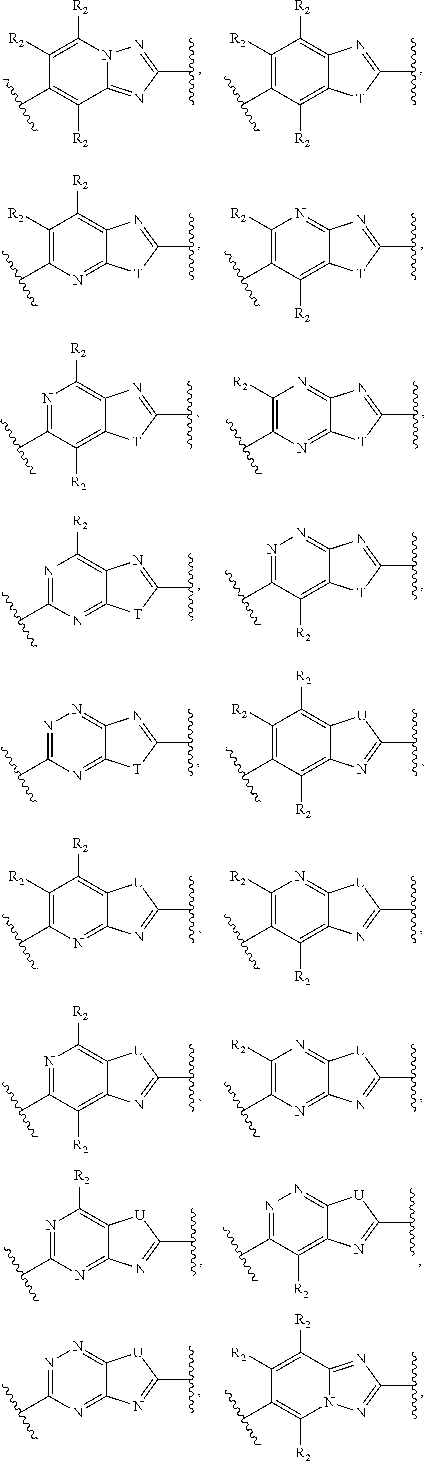 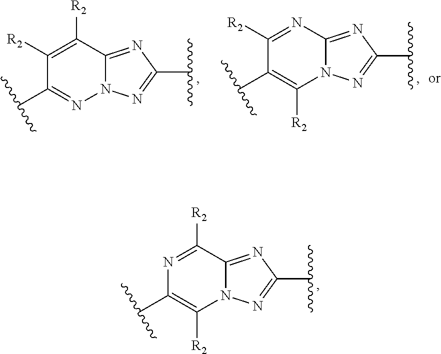
wherein each occurrence of T and U is independently O, N, NRₐ, N(C═O)Rₐ, NC(R_{b})₂OP(═O)(OR_{b})₂, or NSO₂Rₐ where valance permits.

76. The compound of claim 1, wherein the structural moiety 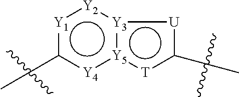
has the structure of 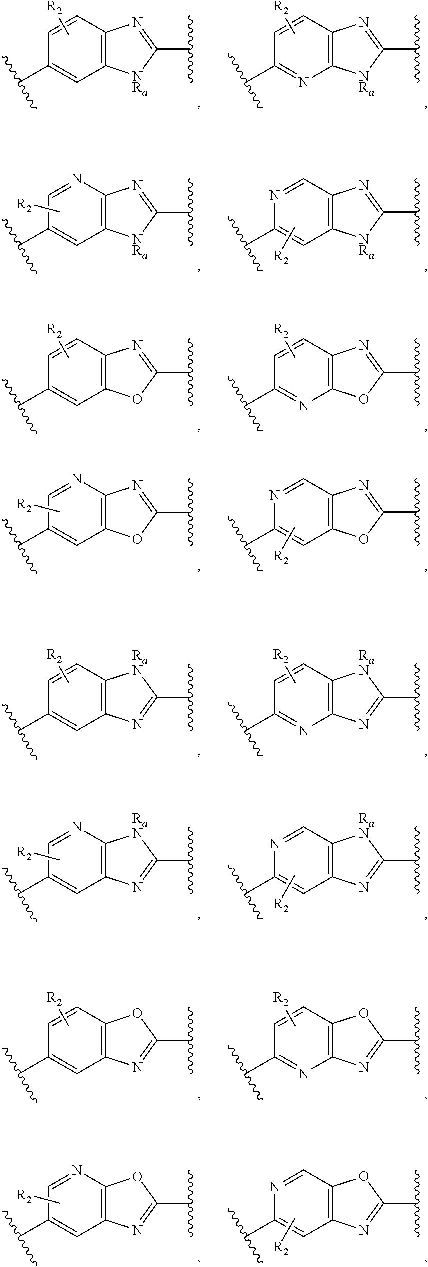 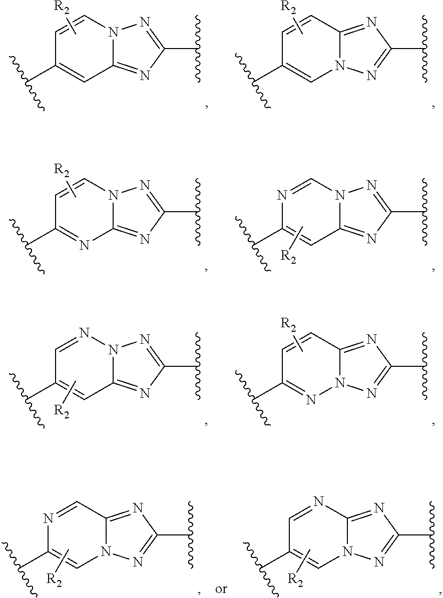
wherein R₂ is H, CH₃, OH, halogen, or NH₂; and wherein Rₐ is H, CH₃, or CH₂CH₃.

77. The compound of claim 1, wherein the structural moiety 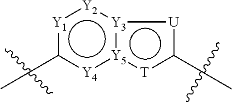
has the structure of

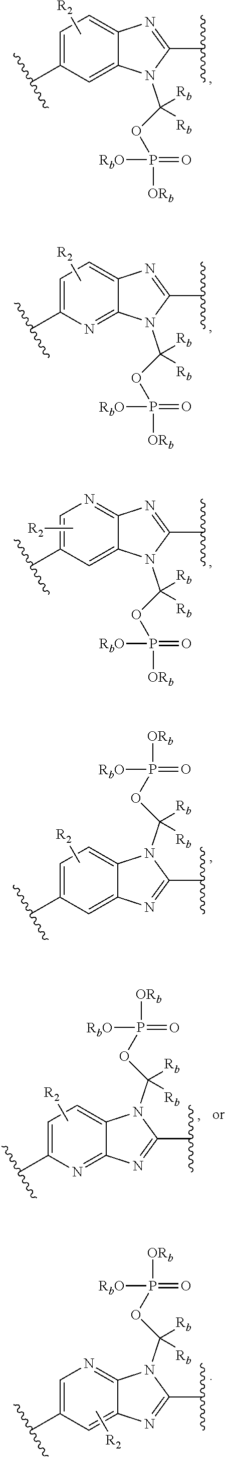

78. The compound of claim 75, wherein each occurrence of R_{b} is independently H or (C₁-C₆)alkyl.

79. The compound of claim 75, wherein each occurrence of R_{b} is independently H, CH₃, CH₂CH₃, or CH(CH₃)₂.

80. The compound of claim 1, wherein each occurrence of R₂ is independently H, CH₃, OH, NH₂, or halogen.

81. The compound of claim 1, wherein the structural moiety 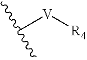
has the structure of

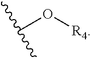

82. The compound of claim 1, wherein the structural moiety 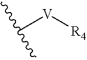
has the structure of

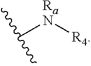

83. The compound of claim 1, wherein the structural moiety 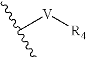
has the structure of

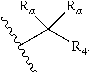

84. The compound of claim 1, wherein the V and R₄ of the structural moiety 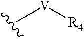
taken together form a (C₄-C₁₀)heterospiroalkyl.

85. The compound of claim 1, wherein V is absent.

86. The compound of claim 1, wherein R₄ is (C₁-C₆)alkyl, 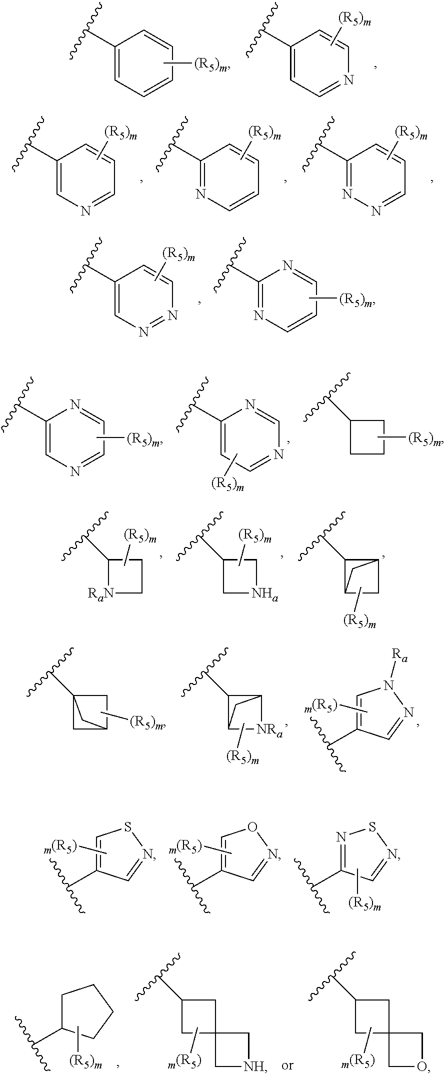
wherein m is an integer from 0-3.

87. The compound of claim 1, wherein each occurrence of R₅ is independently H, (C₁-C₆)alkyl, halogen, ORₐ, OH, NH₂, N(Rₐ)CORₐ, CN, CF₃, (C₁-C₆)haloalkyl, or 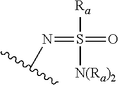
and each occurrence of Rₐ is independently H, (C₂-C₆)alkenyl, or (C₁-C₆)alkyl.

88. The compound of claim 1, wherein the structural moiety 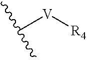
has the structure of 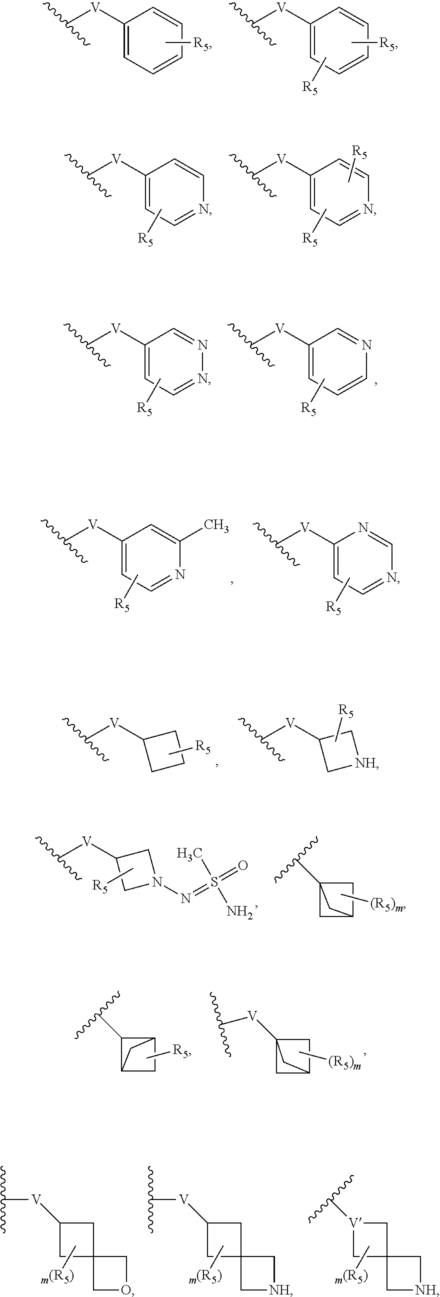 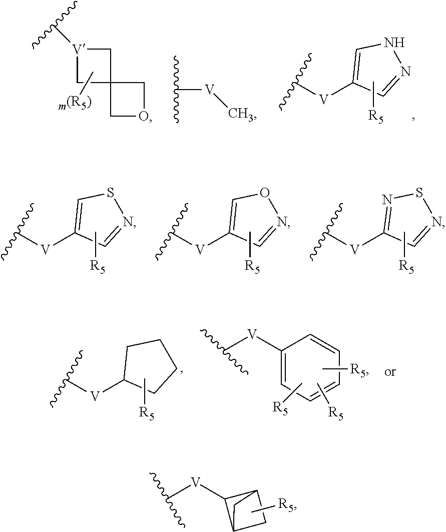
wherein V is C(Rₐ)₂, O, NRₐ, N(C═O)Rₐ, or NSO₂Rₐ and V′ is CRₐ or N.

89. The compound of claim 1, wherein each occurrence of R₅ is independently H, CH₃, halogen, OH, CN, 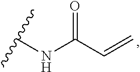 [SMILES: *NC(=O)C=C]
CF₃, (C₁-C₆)haloalkyl, or NH₂.

90. The compound of claim 1, wherein each occurrence of Rₐ is independently H, (C₂-C₆)alkenyl, or (C₁-C₆)alkyl.

91. The compound of claim 1, wherein each occurrence of Rₐ is H, CH₃, or CH₂CH₃.

92. The compound of claim 1, wherein the structural moiety 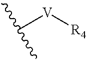
has the structure of

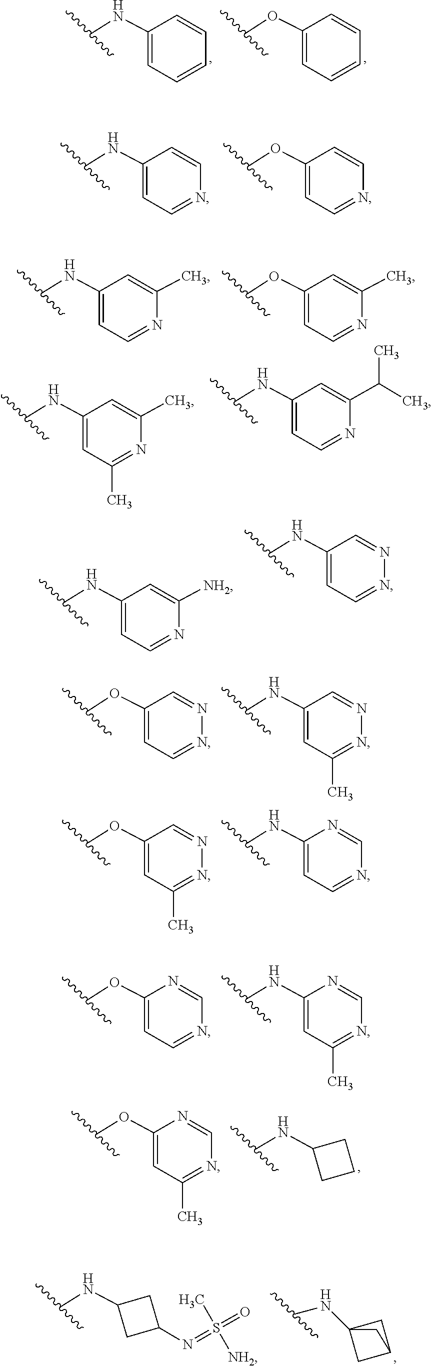

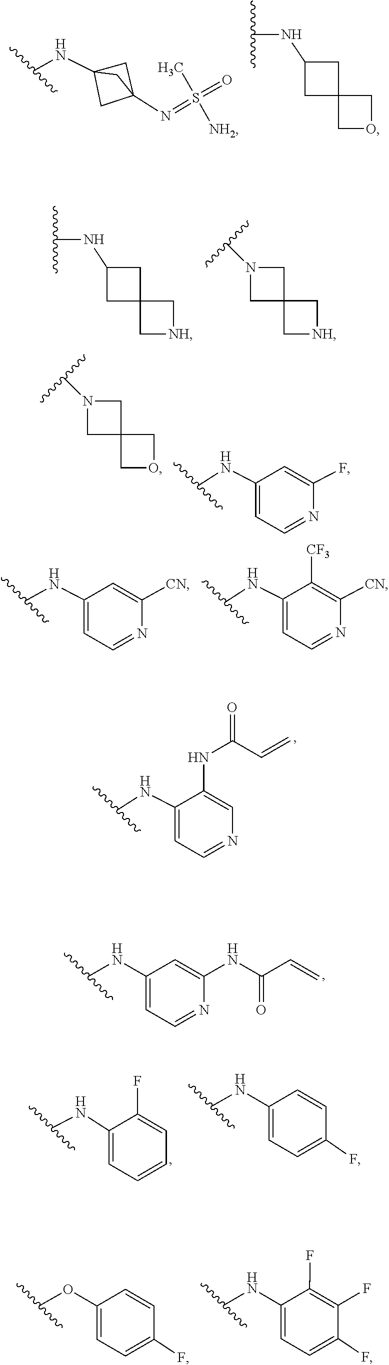

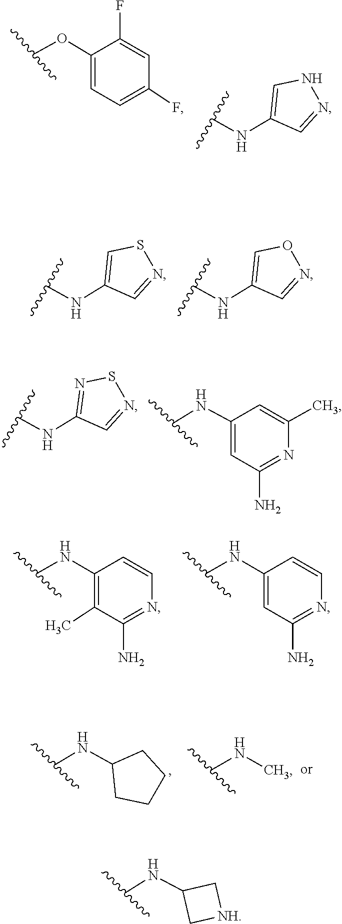

93. The compound of claim 1, wherein the structural moiety 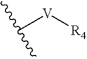
has the structure of

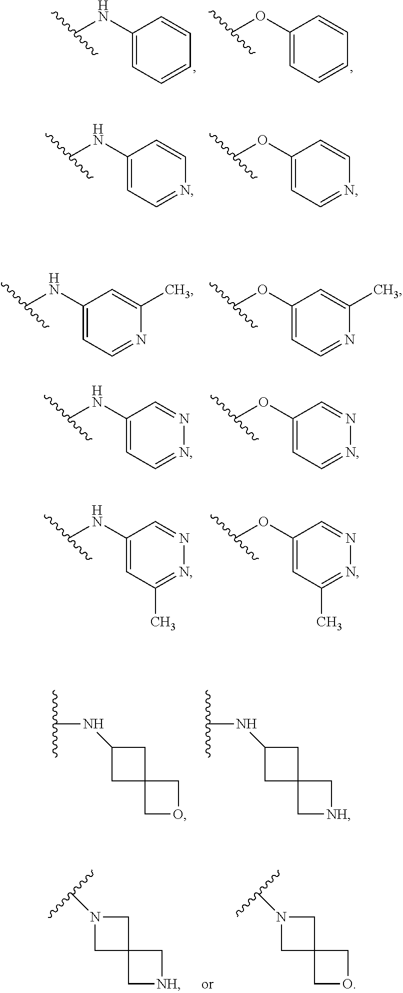

94. The compound of claim 1, wherein the compound of Formula Ia has the structure of 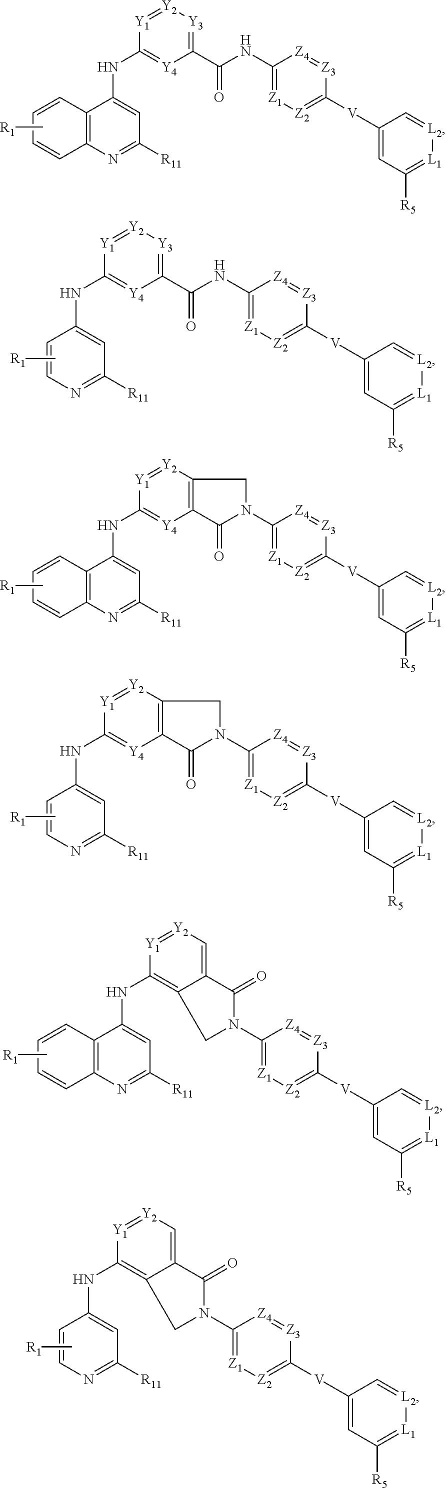 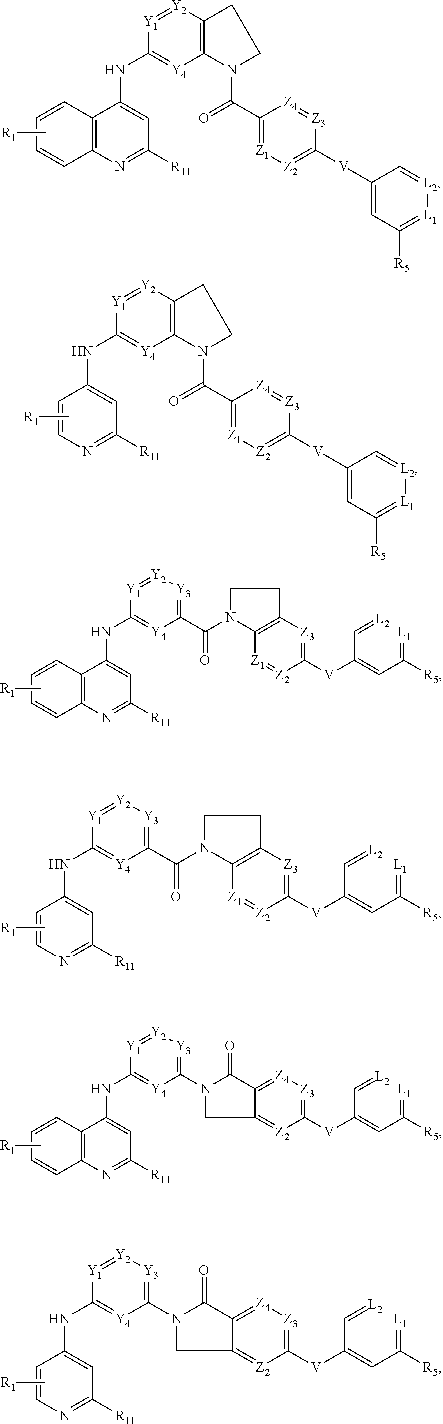 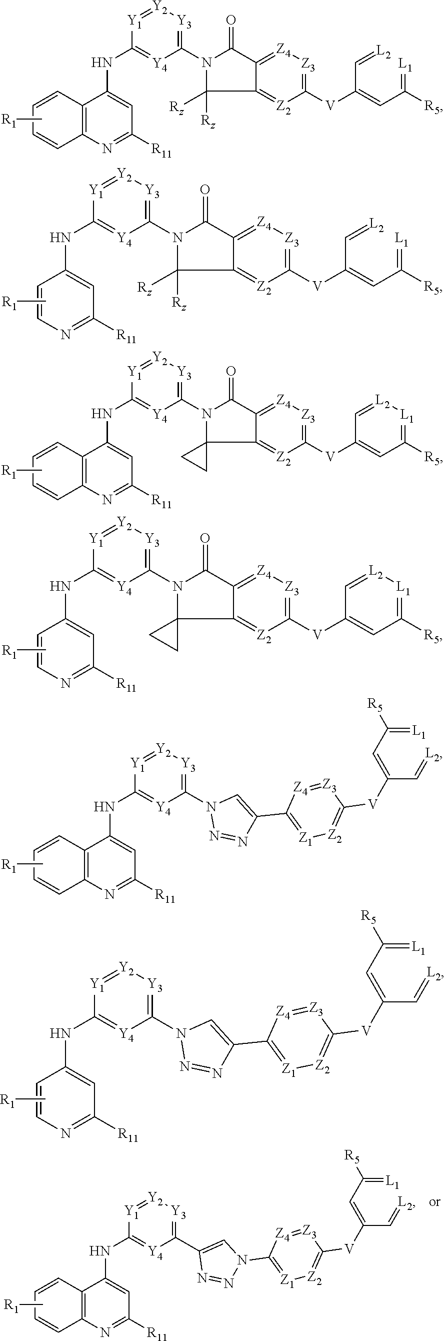 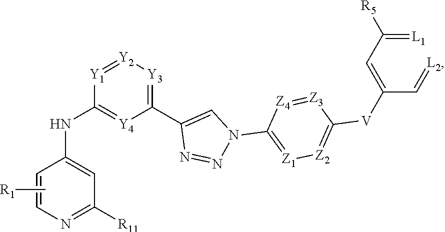
wherein R₁ is H, (C₁-C₆)alkyl, N(Rₐ)₂, (C₃-C₇)heterocycloalkyl, or halogen; R₅ and R₁₁ are each independently H or CH₃; Y₁, Y₂, Y₃, Y₄, Z₁, Z₂, Z₃, Z₄, L₁, and L₂ are each independently CH or N; and V is NH or O.

95. The compound of claim 94, wherein R₁ is H, F, Cl, Br, CH₃, CH₂CH₃, CH(CH₃)₂, NH₂, NMe₂,

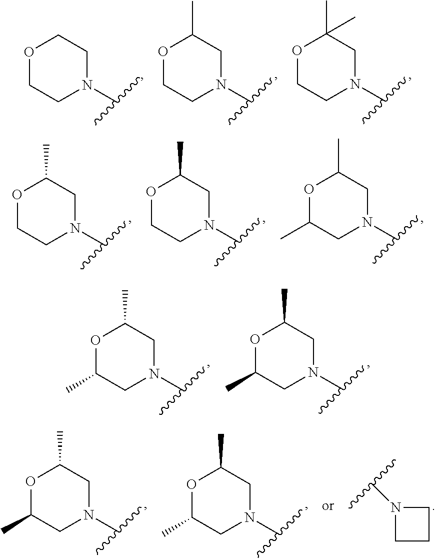

96. The compound of claim 1, wherein the compound of Formula Ib has the structure 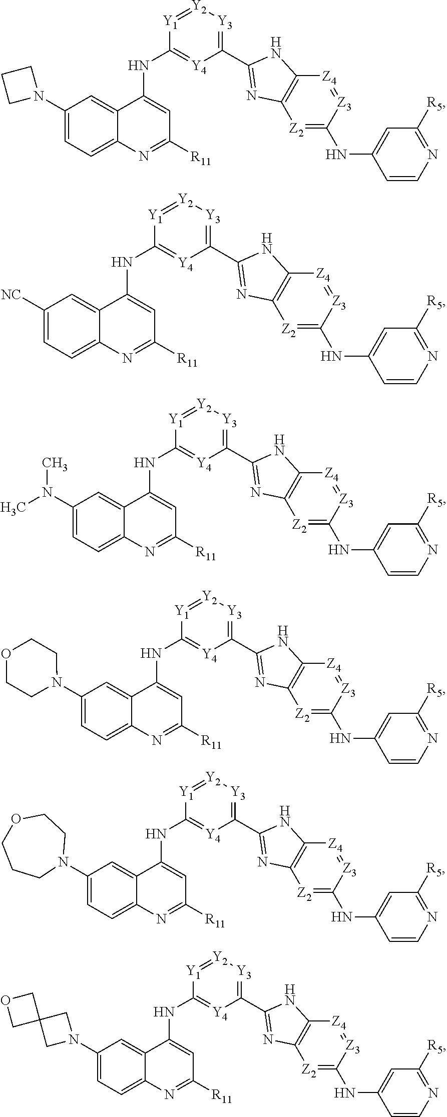
wherein R₁₁ and R₅ are each independently H or CH₃; and Y₁, Y₂, Y₃, Y₄, Z₂, Z₃, and Z₄ are each independently CH or N.

97. The compound of claim 1, wherein the compound of Formula Ib has the structure 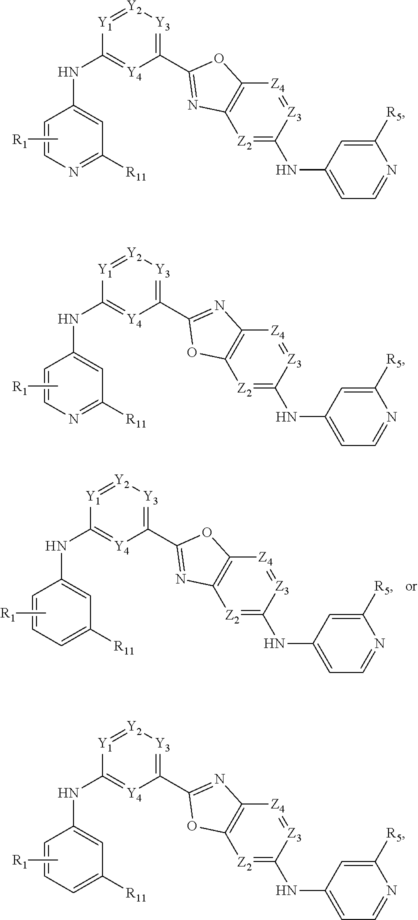
wherein R₁, R₅, and R₁₁ are each independently H, halogen, or CH₃; and Y₁, Y₂, Y₃, Y₄, Z₂, Z₃, and Z₄ are each independently CH or N.

98. The compound of claim 1, wherein the compound of Formula Ic is 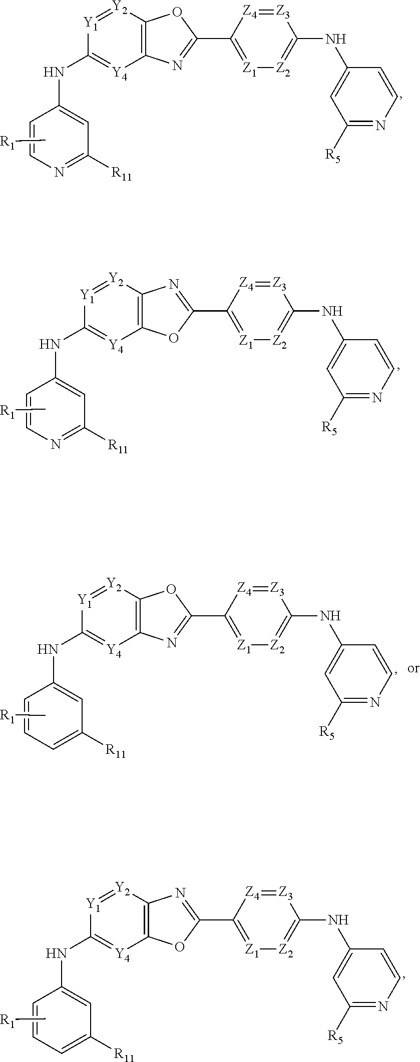
wherein R₁, R₅, and R₁₁ are each independently H, halogen, or CH₃; and Y₁, Y₂, Y₄, Z₄, Z₂, Z₃, and Z₄ are each independently CH or N.

99. The compound of claim 1, wherein the compound of Formula Ia is

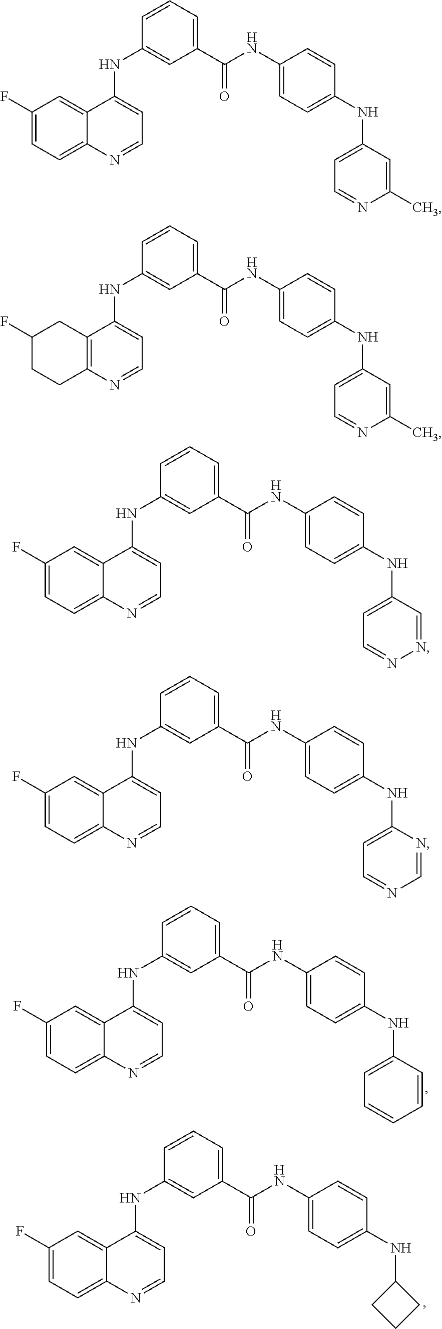 [SMILES: Cc1cc(Nc2ccc(NC(=O)c3cccc(Nc4ccnc5ccc(F)cc45)c3)cc2)ccn1 ; Cc1cc(Nc2ccc(NC(=O)c3cccc(Nc4ccnc5c4CC(F)CC5)c3)cc2)ccn1 ; O=C(Nc1ccc(Nc2ccnnc2)cc1)c1cccc(Nc2ccnc3ccc(F)cc23)c1 ; O=C(Nc1ccc(Nc2ccncn2)cc1)c1cccc(Nc2ccnc3ccc(F)cc23)c1 ; O=C(Nc1ccc(Nc2ccccc2)cc1)c1cccc(Nc2ccnc3ccc(F)cc23)c1 ; O=C(Nc1ccc(NC2CCC2)cc1)c1cccc(Nc2ccnc3ccc(F)cc23)c1]

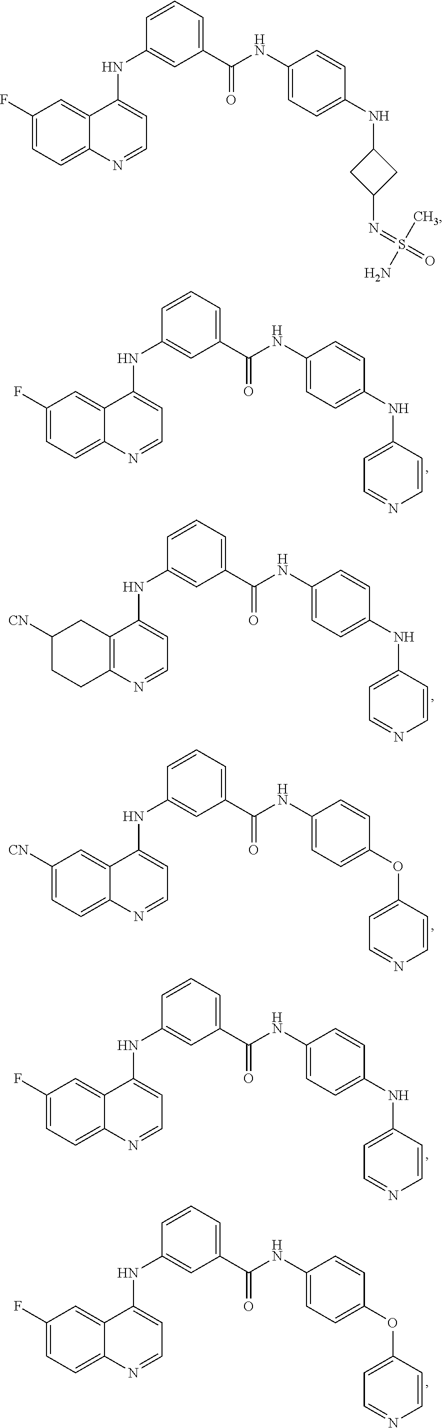

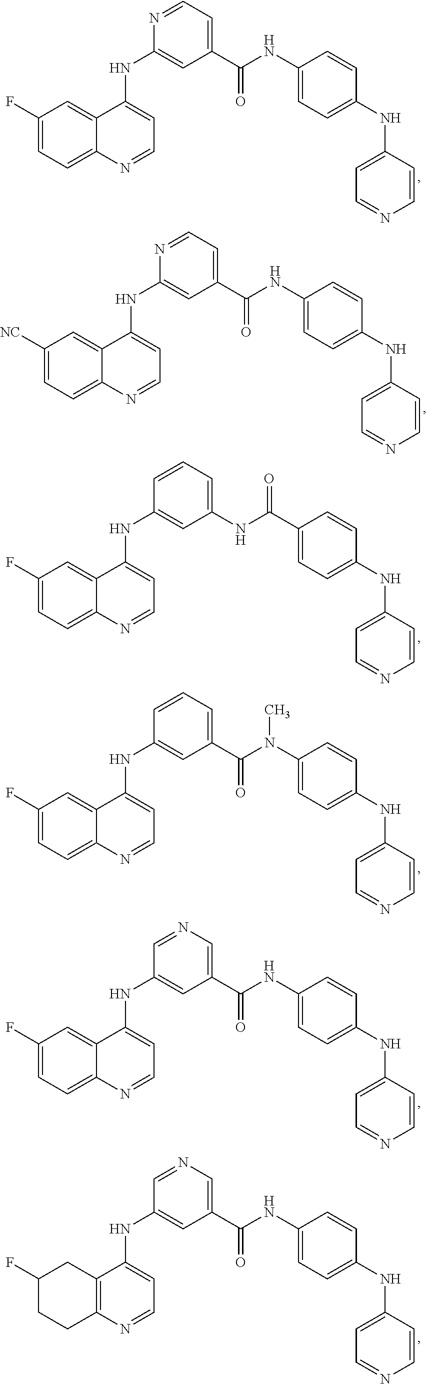 [SMILES: O=C(Nc1ccc(Nc2ccncc2)cc1)c1ccnc(Nc2ccnc3ccc(F)cc23)c1 ; N#Cc1ccc2nccc(Nc3cc(C(=O)Nc4ccc(Nc5ccncc5)cc4)ccn3)c2c1 ; O=C(Nc1cccc(Nc2ccnc3ccc(F)cc23)c1)c1ccc(Nc2ccncc2)cc1 ; CN(C(=O)c1cccc(Nc2ccnc3ccc(F)cc23)c1)c1ccc(Nc2ccncc2)cc1 ; O=C(Nc1ccc(Nc2ccncc2)cc1)c1cncc(Nc2ccnc3ccc(F)cc23)c1 ; O=C(Nc1ccc(Nc2ccncc2)cc1)c1cncc(Nc2ccnc3c2CC(F)CC3)c1]

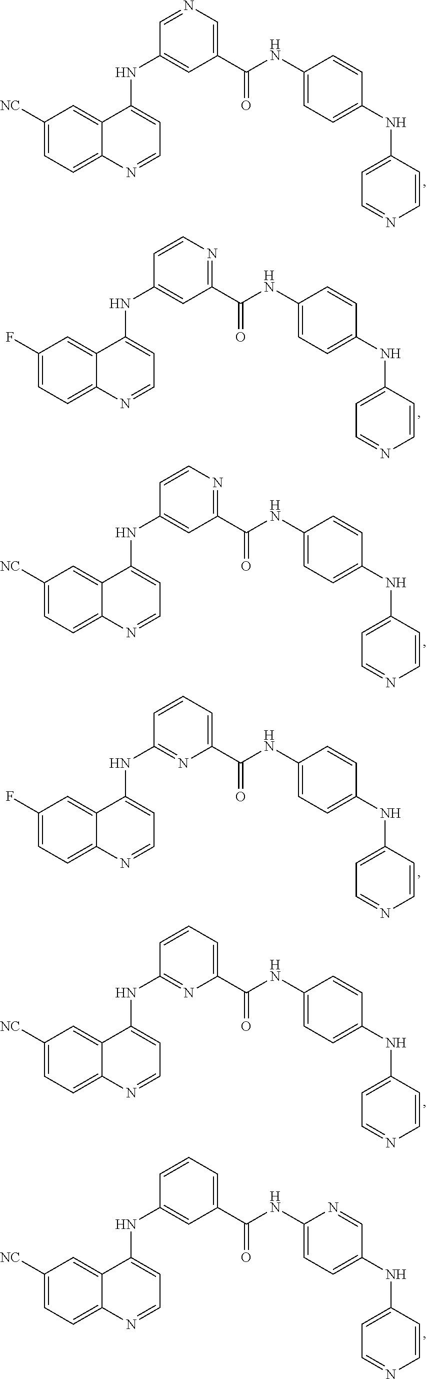 [SMILES: N#Cc1ccc2nccc(Nc3cncc(C(=O)Nc4ccc(Nc5ccncc5)cc4)c3)c2c1 ; O=C(Nc1ccc(Nc2ccncc2)cc1)c1cc(Nc2ccnc3ccc(F)cc23)ccn1 ; N#Cc1ccc2nccc(Nc3ccnc(C(=O)Nc4ccc(Nc5ccncc5)cc4)c3)c2c1 ; O=C(Nc1ccc(Nc2ccncc2)cc1)c1cccc(Nc2ccnc3ccc(F)cc23)n1 ; N#Cc1ccc2nccc(Nc3cccc(C(=O)Nc4ccc(Nc5ccncc5)cc4)n3)c2c1 ; N#Cc1ccc2nccc(Nc3cccc(C(=O)Nc4ccc(Nc5ccncc5)cn4)c3)c2c1]

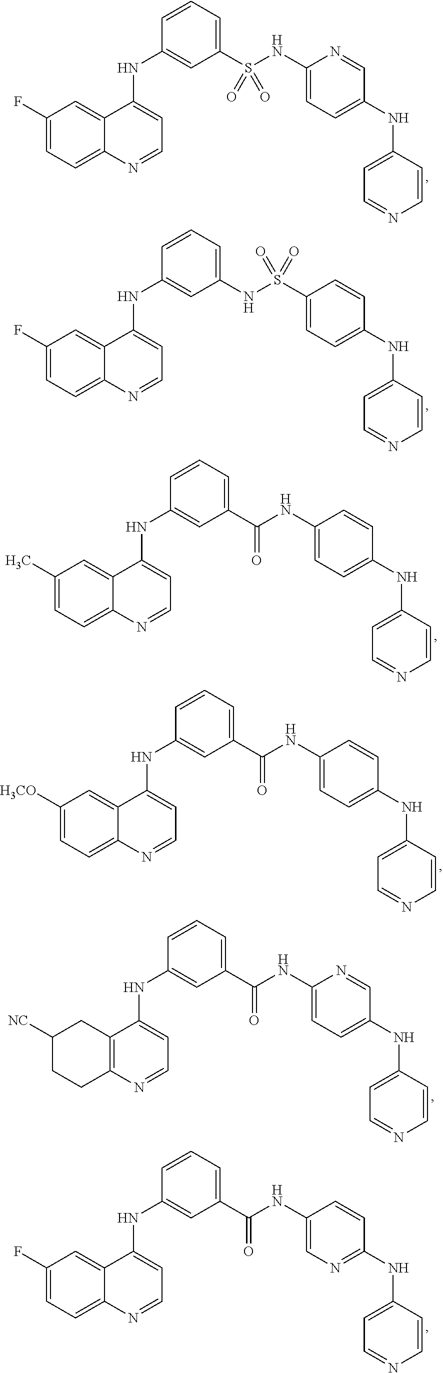 [SMILES: O=S(=O)(Nc1ccc(Nc2ccncc2)cn1)c1cccc(Nc2ccnc3ccc(F)cc23)c1 ; O=S(=O)(Nc1cccc(Nc2ccnc3ccc(F)cc23)c1)c1ccc(Nc2ccncc2)cc1 ; Cc1ccc2nccc(Nc3cccc(C(=O)Nc4ccc(Nc5ccncc5)cc4)c3)c2c1 ; COc1ccc2nccc(Nc3cccc(C(=O)Nc4ccc(Nc5ccncc5)cc4)c3)c2c1 ; N#CC1CCc2nccc(Nc3cccc(C(=O)Nc4ccc(Nc5ccncc5)cn4)c3)c2C1 ; O=C(Nc1ccc(Nc2ccncc2)nc1)c1cccc(Nc2ccnc3ccc(F)cc23)c1]

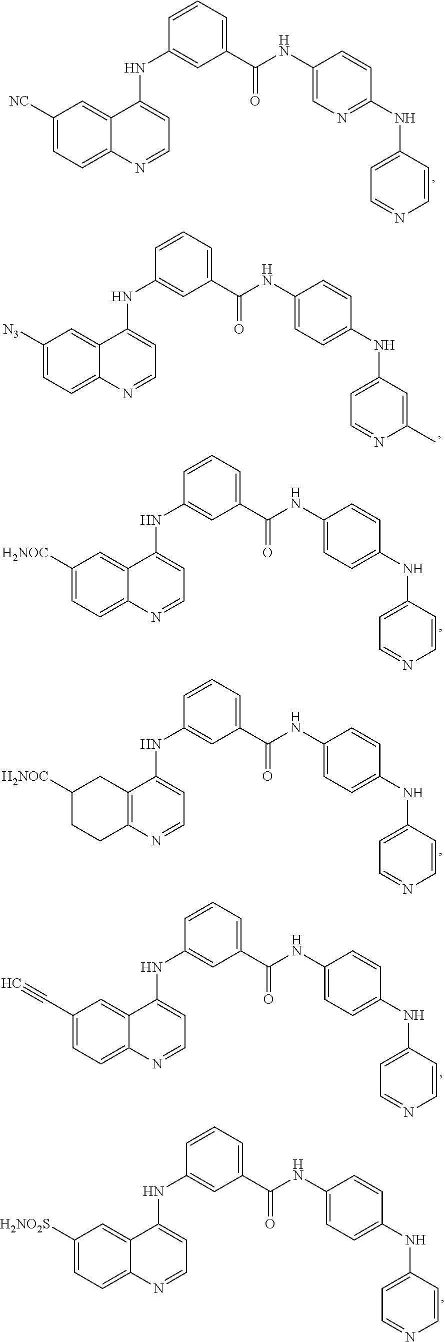

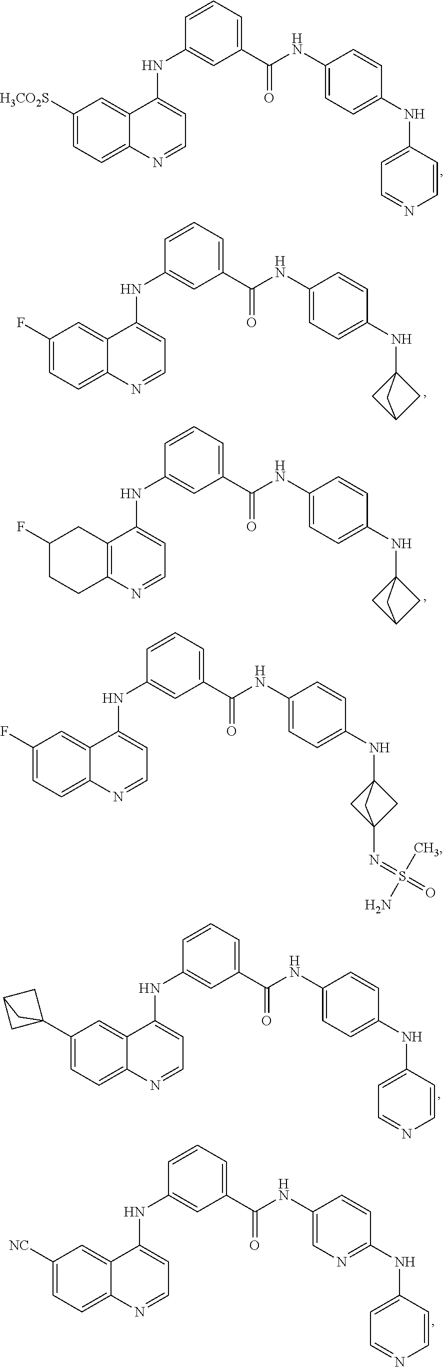

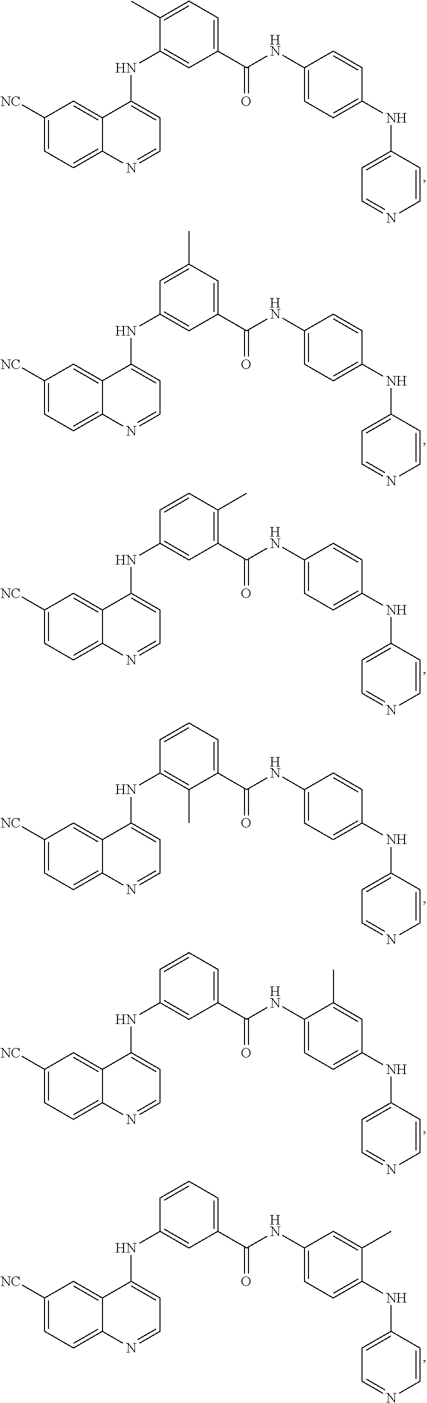 [SMILES: Cc1ccc(C(=O)Nc2ccc(Nc3ccncc3)cc2)cc1Nc1ccnc2ccc(C#N)cc12 ; Cc1cc(Nc2ccnc3ccc(C#N)cc23)cc(C(=O)Nc2ccc(Nc3ccncc3)cc2)c1 ; Cc1ccc(Nc2ccnc3ccc(C#N)cc23)cc1C(=O)Nc1ccc(Nc2ccncc2)cc1 ; Cc1c(Nc2ccnc3ccc(C#N)cc23)cccc1C(=O)Nc1ccc(Nc2ccncc2)cc1 ; Cc1cc(Nc2ccncc2)ccc1NC(=O)c1cccc(Nc2ccnc3ccc(C#N)cc23)c1 ; Cc1cc(NC(=O)c2cccc(Nc3ccnc4ccc(C#N)cc34)c2)ccc1Nc1ccncc1]

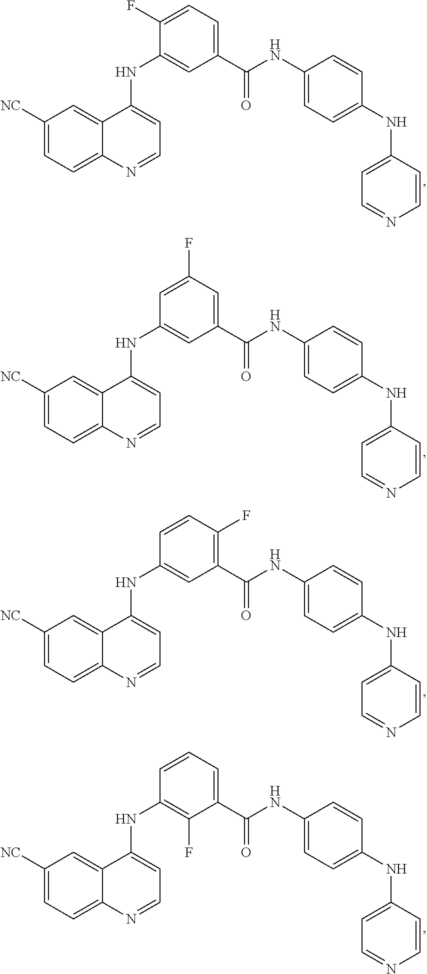 [SMILES: N#Cc1ccc2nccc(Nc3cc(C(=O)Nc4ccc(Nc5ccncc5)cc4)ccc3F)c2c1 ; N#Cc1ccc2nccc(Nc3cc(F)cc(C(=O)Nc4ccc(Nc5ccncc5)cc4)c3)c2c1 ; N#Cc1ccc2nccc(Nc3ccc(F)c(C(=O)Nc4ccc(Nc5ccncc5)cc4)c3)c2c1 ; N#Cc1ccc2nccc(Nc3cccc(C(=O)Nc4ccc(Nc5ccncc5)cc4)c3F)c2c1]

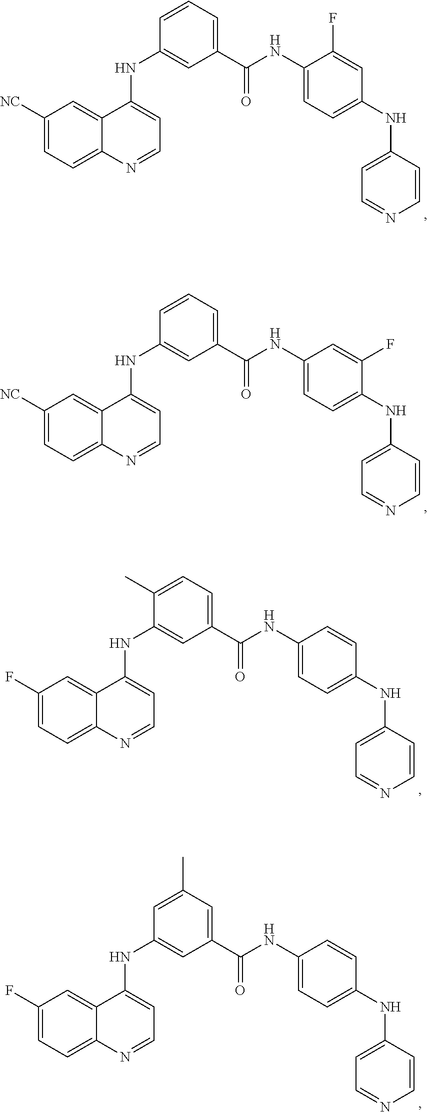 [SMILES: N#Cc1ccc2nccc(Nc3cccc(C(=O)Nc4ccc(Nc5ccncc5)cc4F)c3)c2c1 ; N#Cc1ccc2nccc(Nc3cccc(C(=O)Nc4ccc(Nc5ccncc5)c(F)c4)c3)c2c1 ; Cc1ccc(C(=O)Nc2ccc(Nc3ccncc3)cc2)cc1Nc1ccnc2ccc(F)cc12 ; Cc1cc(Nc2ccnc3ccc(F)cc23)cc(C(=O)Nc2ccc(Nc3ccncc3)cc2)c1]

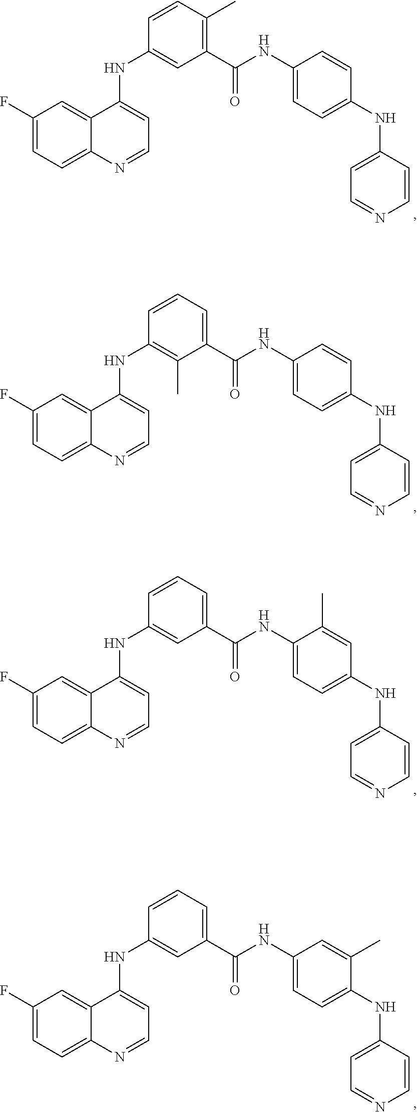 [SMILES: Cc1ccc(Nc2ccnc3ccc(F)cc23)cc1C(=O)Nc1ccc(Nc2ccncc2)cc1 ; Cc1c(Nc2ccnc3ccc(F)cc23)cccc1C(=O)Nc1ccc(Nc2ccncc2)cc1 ; Cc1cc(Nc2ccncc2)ccc1NC(=O)c1cccc(Nc2ccnc3ccc(F)cc23)c1 ; Cc1cc(NC(=O)c2cccc(Nc3ccnc4ccc(F)cc34)c2)ccc1Nc1ccncc1]

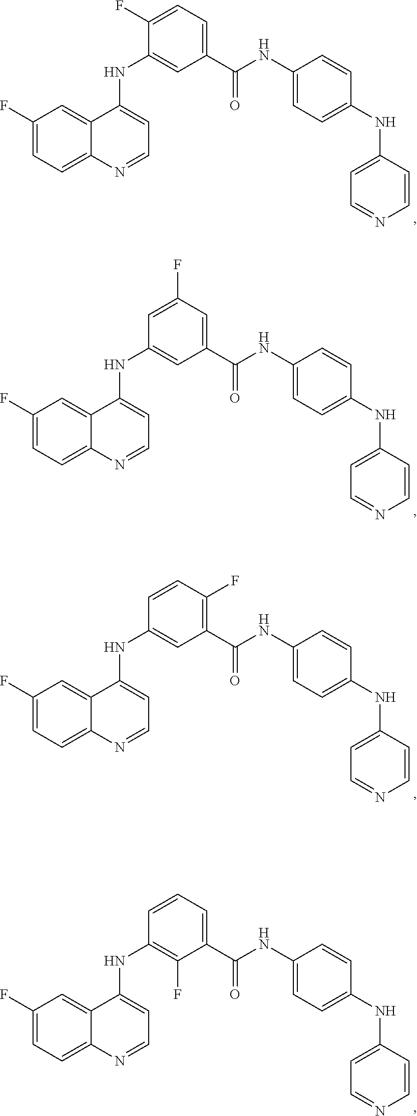 [SMILES: O=C(Nc1ccc(Nc2ccncc2)cc1)c1ccc(F)c(Nc2ccnc3ccc(F)cc23)c1 ; O=C(Nc1ccc(Nc2ccncc2)cc1)c1cc(F)cc(Nc2ccnc3ccc(F)cc23)c1 ; O=C(Nc1ccc(Nc2ccncc2)cc1)c1cc(Nc2ccnc3ccc(F)cc23)ccc1F ; O=C(Nc1ccc(Nc2ccncc2)cc1)c1cccc(Nc2ccnc3ccc(F)cc23)c1F]

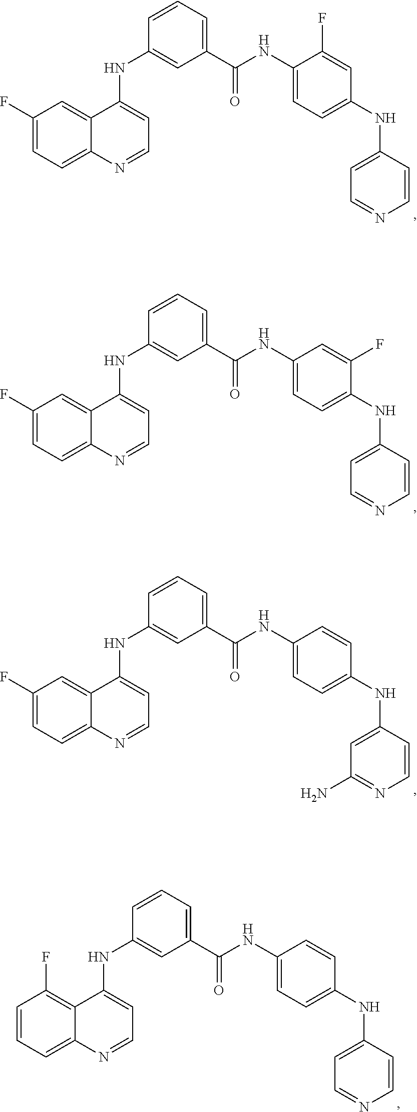 [SMILES: O=C(Nc1ccc(Nc2ccncc2)cc1F)c1cccc(Nc2ccnc3ccc(F)cc23)c1 ; O=C(Nc1ccc(Nc2ccncc2)c(F)c1)c1cccc(Nc2ccnc3ccc(F)cc23)c1 ; Nc1cc(Nc2ccc(NC(=O)c3cccc(Nc4ccnc5ccc(F)cc45)c3)cc2)ccn1 ; O=C(Nc1ccc(Nc2ccncc2)cc1)c1cccc(Nc2ccnc3cccc(F)c23)c1]

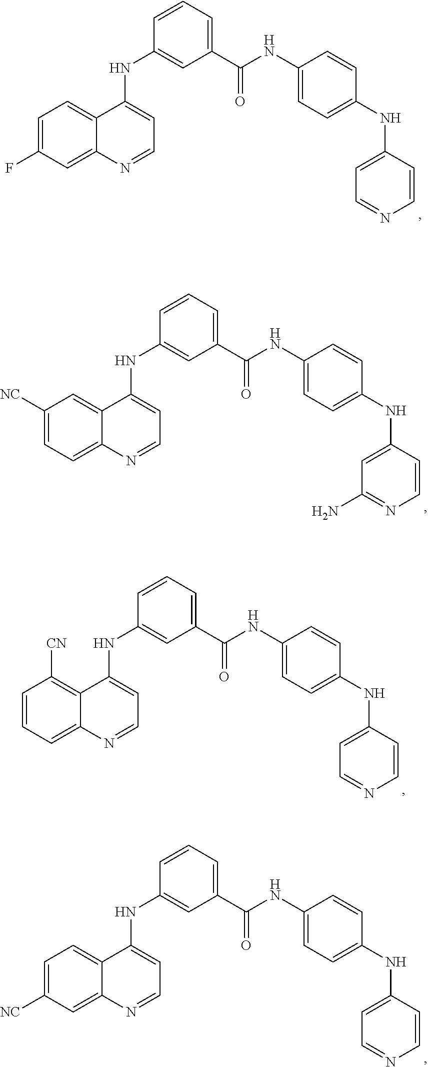 [SMILES: O=C(Nc1ccc(Nc2ccncc2)cc1)c1cccc(Nc2ccnc3cc(F)ccc23)c1 ; N#Cc1ccc2nccc(Nc3cccc(C(=O)Nc4ccc(Nc5ccnc(N)c5)cc4)c3)c2c1 ; N#Cc1cccc2nccc(Nc3cccc(C(=O)Nc4ccc(Nc5ccncc5)cc4)c3)c12 ; N#Cc1ccc2c(Nc3cccc(C(=O)Nc4ccc(Nc5ccncc5)cc4)c3)ccnc2c1]

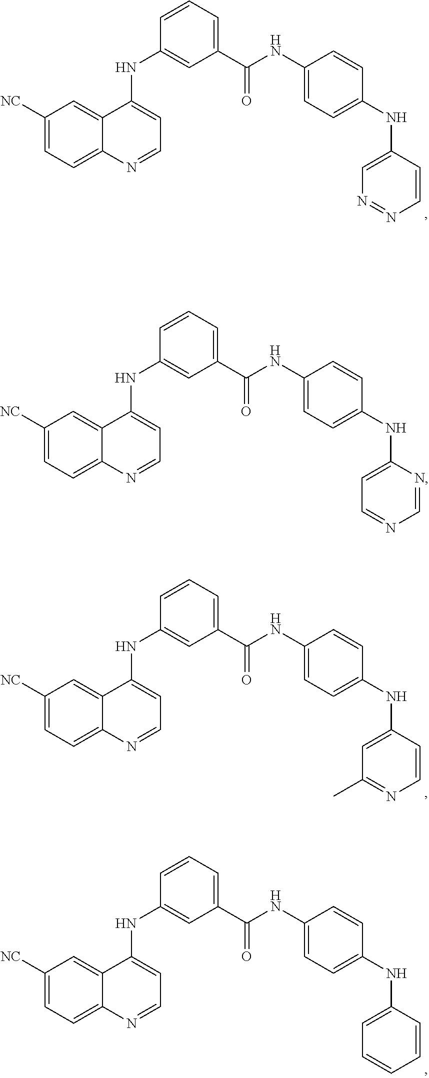 [SMILES: N#Cc1ccc2nccc(Nc3cccc(C(=O)Nc4ccc(Nc5ccnnc5)cc4)c3)c2c1 ; N#Cc1ccc2nccc(Nc3cccc(C(=O)Nc4ccc(Nc5ccncn5)cc4)c3)c2c1 ; Cc1cc(Nc2ccc(NC(=O)c3cccc(Nc4ccnc5ccc(C#N)cc45)c3)cc2)ccn1 ; N#Cc1ccc2nccc(Nc3cccc(C(=O)Nc4ccc(Nc5ccccc5)cc4)c3)c2c1]

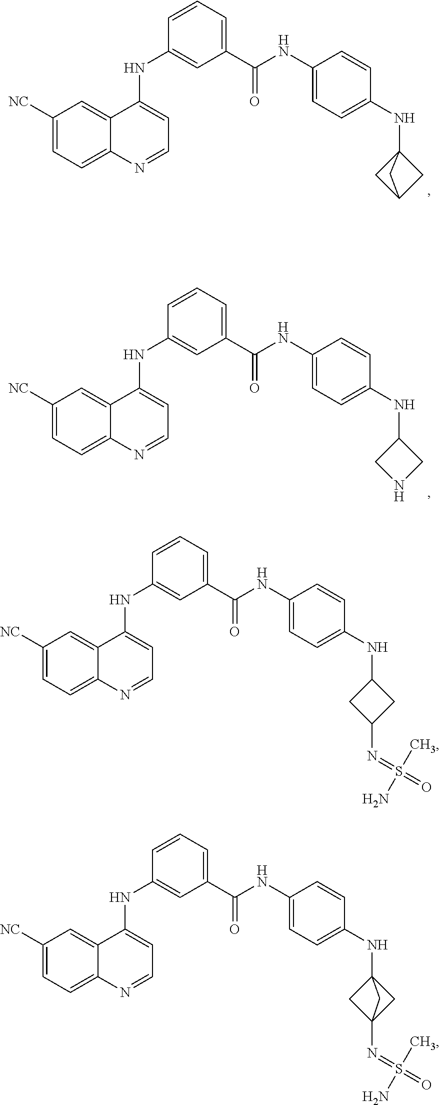 [SMILES: N#Cc1ccc2nccc(Nc3cccc(C(=O)Nc4ccc(NC56CC(C5)C6)cc4)c3)c2c1 ; N#Cc1ccc2nccc(Nc3cccc(C(=O)Nc4ccc(NC5CNC5)cc4)c3)c2c1 ; CS(N)(=O)=NC1CC(Nc2ccc(NC(=O)c3cccc(Nc4ccnc5ccc(C#N)cc45)c3)cc2)C1 ; CS(N)(=O)=NC12CC(Nc3ccc(NC(=O)c4cccc(Nc5ccnc6ccc(C#N)cc56)c4)cc3)(C1)C2]

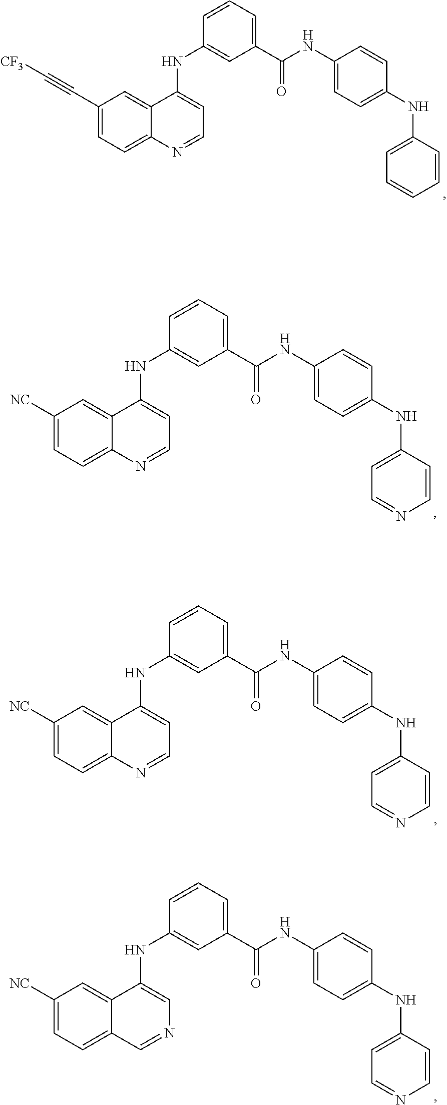 [SMILES: O=C(Nc1ccc(Nc2ccccc2)cc1)c1cccc(Nc2ccnc3ccc(C#CC(F)(F)F)cc23)c1 ; N#Cc1ccc2nccc(Nc3cccc(C(=O)Nc4ccc(Nc5ccncc5)cc4)c3)c2c1 ; N#Cc1ccc2nccc(Nc3cccc(C(=O)Nc4ccc(Nc5ccncc5)cc4)c3)c2c1 ; N#Cc1ccc2cncc(Nc3cccc(C(=O)Nc4ccc(Nc5ccncc5)cc4)c3)c2c1]

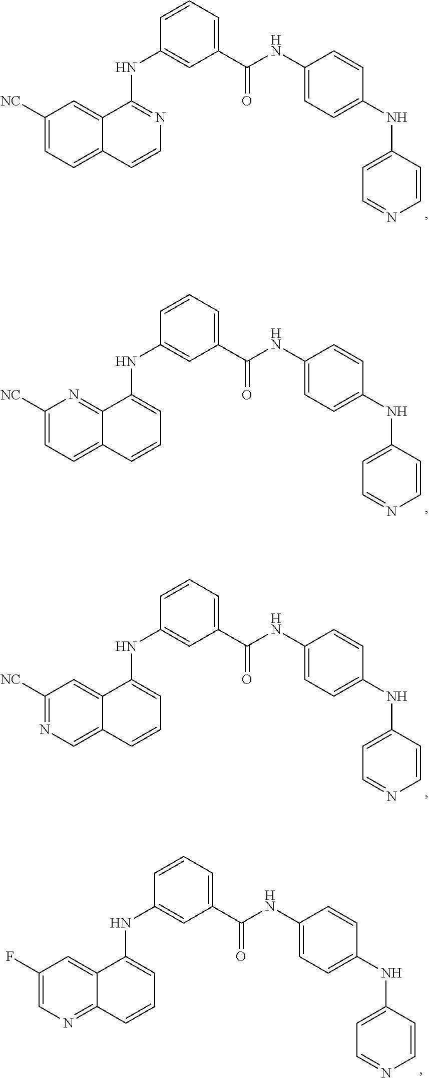 [SMILES: N#Cc1ccc2ccnc(Nc3cccc(C(=O)Nc4ccc(Nc5ccncc5)cc4)c3)c2c1 ; N#Cc1ccc2cccc(Nc3cccc(C(=O)Nc4ccc(Nc5ccncc5)cc4)c3)c2n1 ; N#Cc1cc2c(Nc3cccc(C(=O)Nc4ccc(Nc5ccncc5)cc4)c3)cccc2cn1 ; O=C(Nc1ccc(Nc2ccncc2)cc1)c1cccc(Nc2cccc3ncc(F)cc23)c1]

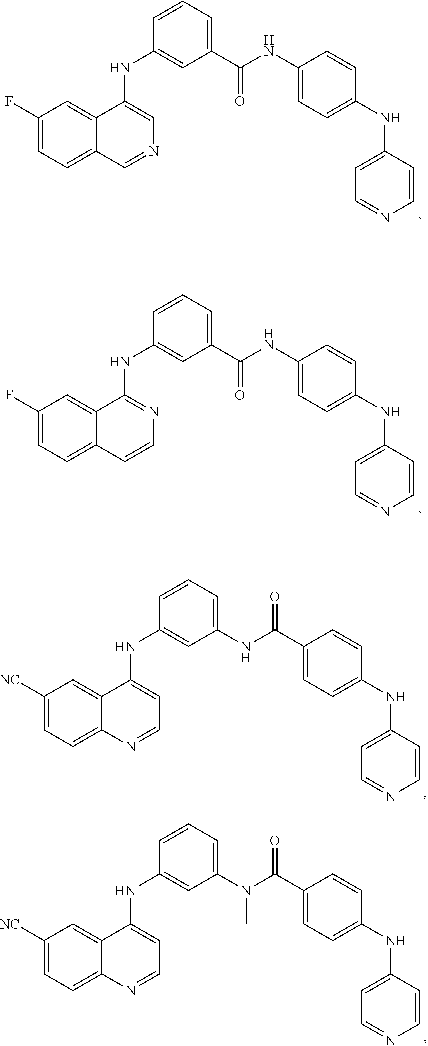 [SMILES: O=C(Nc1ccc(Nc2ccncc2)cc1)c1cccc(Nc2cncc3ccc(F)cc23)c1 ; O=C(Nc1ccc(Nc2ccncc2)cc1)c1cccc(Nc2nccc3ccc(F)cc23)c1 ; N#Cc1ccc2nccc(Nc3cccc(NC(=O)c4ccc(Nc5ccncc5)cc4)c3)c2c1 ; CN(C(=O)c1ccc(Nc2ccncc2)cc1)c1cccc(Nc2ccnc3ccc(C#N)cc23)c1]

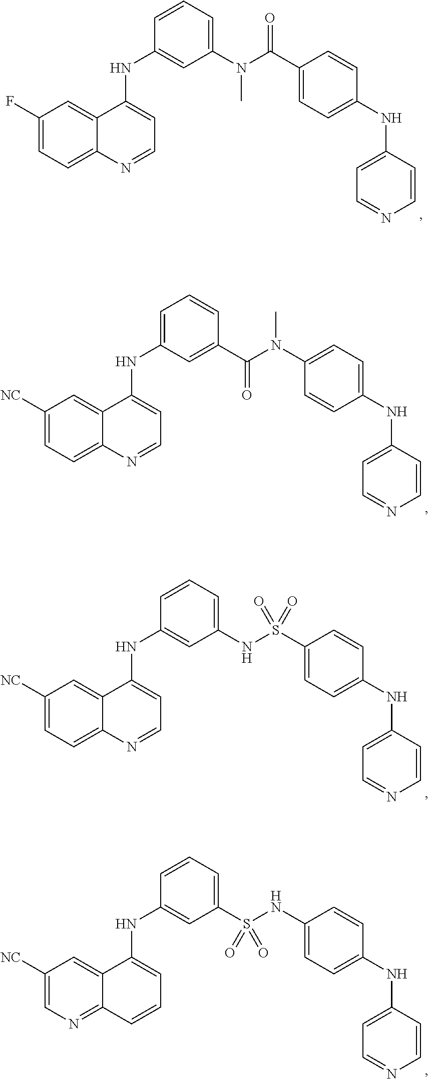 [SMILES: CN(C(=O)c1ccc(Nc2ccncc2)cc1)c1cccc(Nc2ccnc3ccc(F)cc23)c1 ; CN(C(=O)c1cccc(Nc2ccnc3ccc(C#N)cc23)c1)c1ccc(Nc2ccncc2)cc1 ; N#Cc1ccc2nccc(Nc3cccc(NS(=O)(=O)c4ccc(Nc5ccncc5)cc4)c3)c2c1 ; N#Cc1cnc2cccc(Nc3cccc(S(=O)(=O)Nc4ccc(Nc5ccncc5)cc4)c3)c2c1]

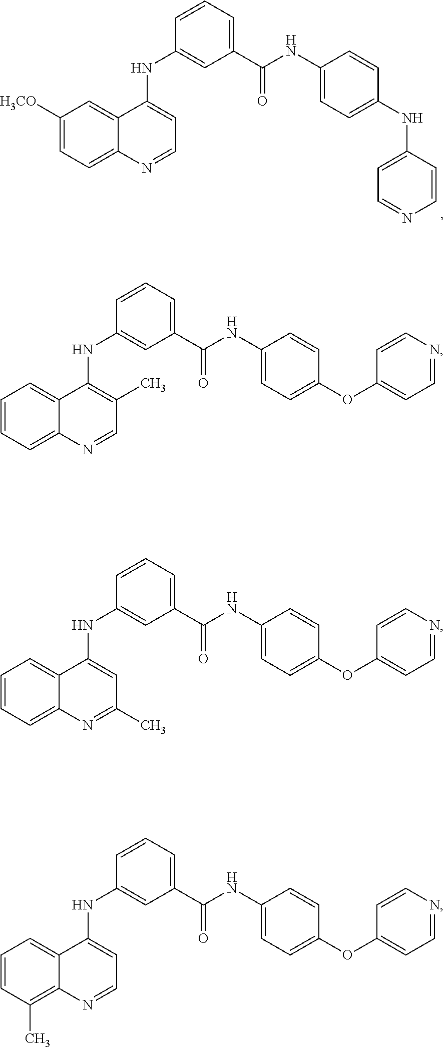 [SMILES: COc1ccc2nccc(Nc3cccc(C(=O)Nc4ccc(Nc5ccncc5)cc4)c3)c2c1 ; Cc1cnc2ccccc2c1Nc1cccc(C(=O)Nc2ccc(Oc3ccncc3)cc2)c1 ; Cc1cc(Nc2cccc(C(=O)Nc3ccc(Oc4ccncc4)cc3)c2)c2ccccc2n1 ; Cc1cccc2c(Nc3cccc(C(=O)Nc4ccc(Oc5ccncc5)cc4)c3)ccnc12]

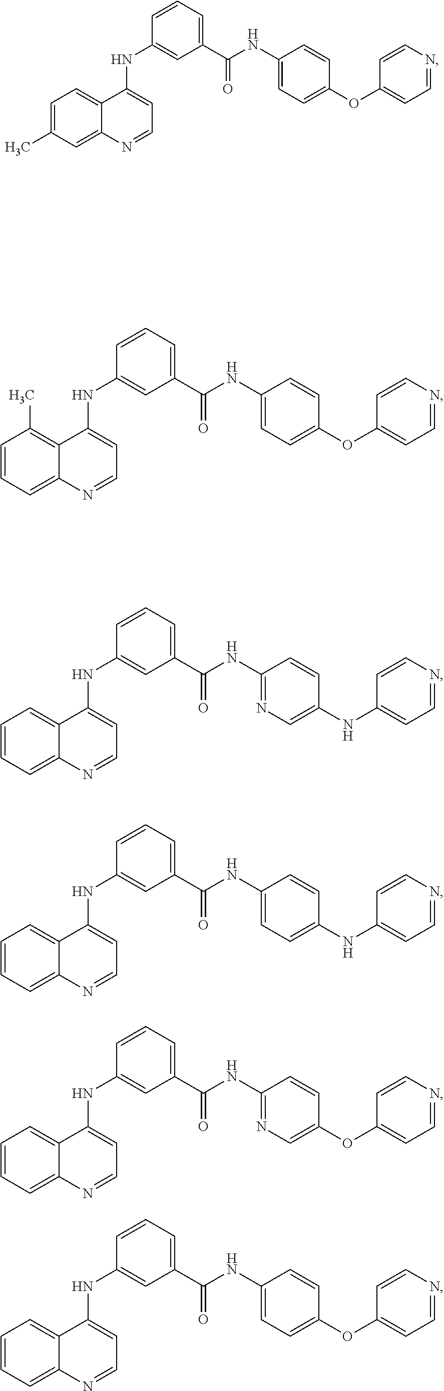 [SMILES: Cc1ccc2c(Nc3cccc(C(=O)Nc4ccc(Oc5ccncc5)cc4)c3)ccnc2c1 ; Cc1cccc2nccc(Nc3cccc(C(=O)Nc4ccc(Oc5ccncc5)cc4)c3)c12 ; O=C(Nc1ccc(Nc2ccncc2)cn1)c1cccc(Nc2ccnc3ccccc23)c1 ; O=C(Nc1ccc(Nc2ccncc2)cc1)c1cccc(Nc2ccnc3ccccc23)c1 ; O=C(Nc1ccc(Oc2ccncc2)cn1)c1cccc(Nc2ccnc3ccccc23)c1 ; O=C(Nc1ccc(Oc2ccncc2)cc1)c1cccc(Nc2ccnc3ccccc23)c1]

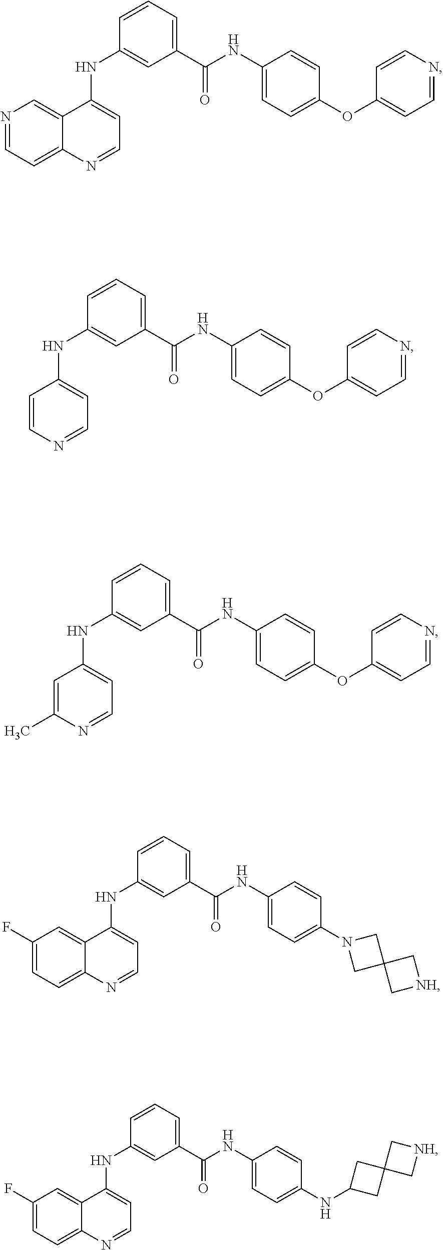 [SMILES: O=C(Nc1ccc(Oc2ccncc2)cc1)c1cccc(Nc2ccnc3ccncc23)c1 ; O=C(Nc1ccc(Oc2ccncc2)cc1)c1cccc(Nc2ccncc2)c1 ; Cc1cc(Nc2cccc(C(=O)Nc3ccc(Oc4ccncc4)cc3)c2)ccn1 ; O=C(Nc1ccc(N2CC3(CNC3)C2)cc1)c1cccc(Nc2ccnc3ccc(F)cc23)c1 ; O=C(Nc1ccc(NC2CC3(CNC3)C2)cc1)c1cccc(Nc2ccnc3ccc(F)cc23)c1]

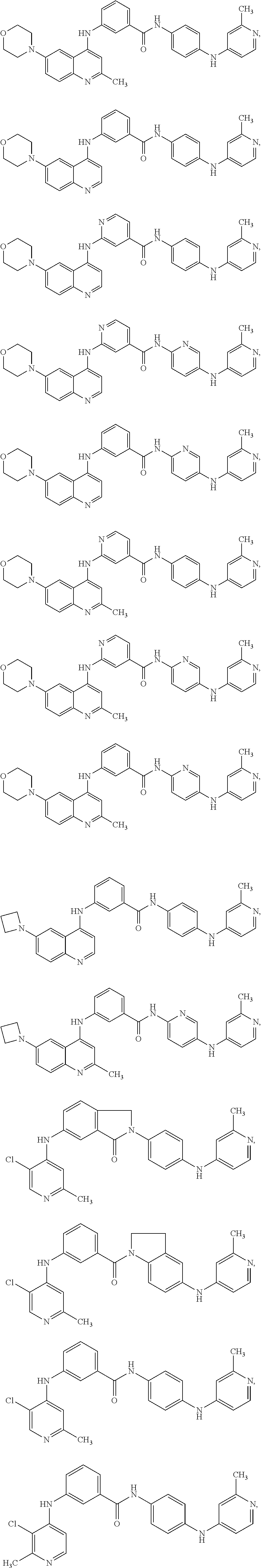

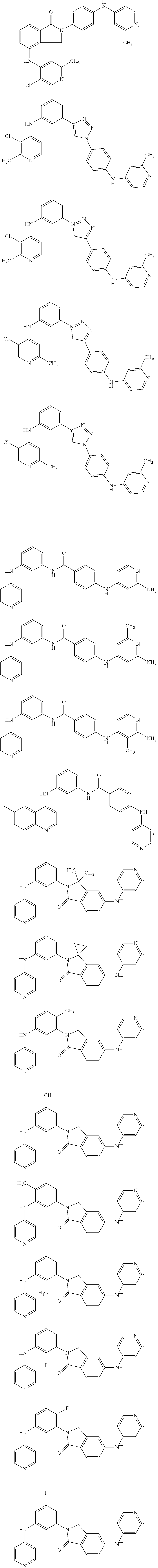

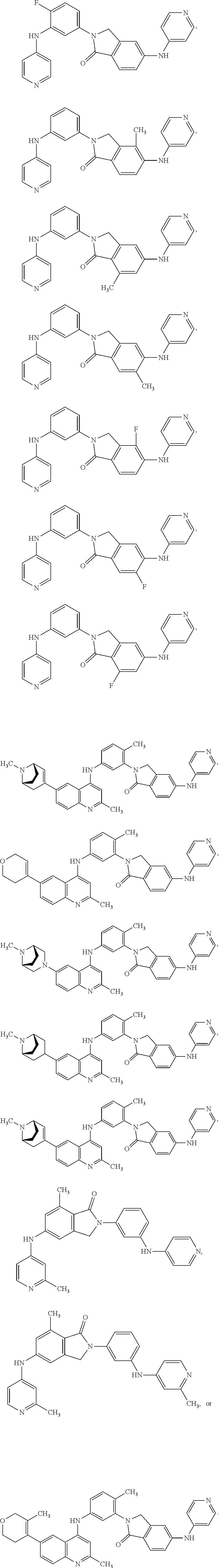

100. The compound of claim 1, wherein the compound of Formula Ib is

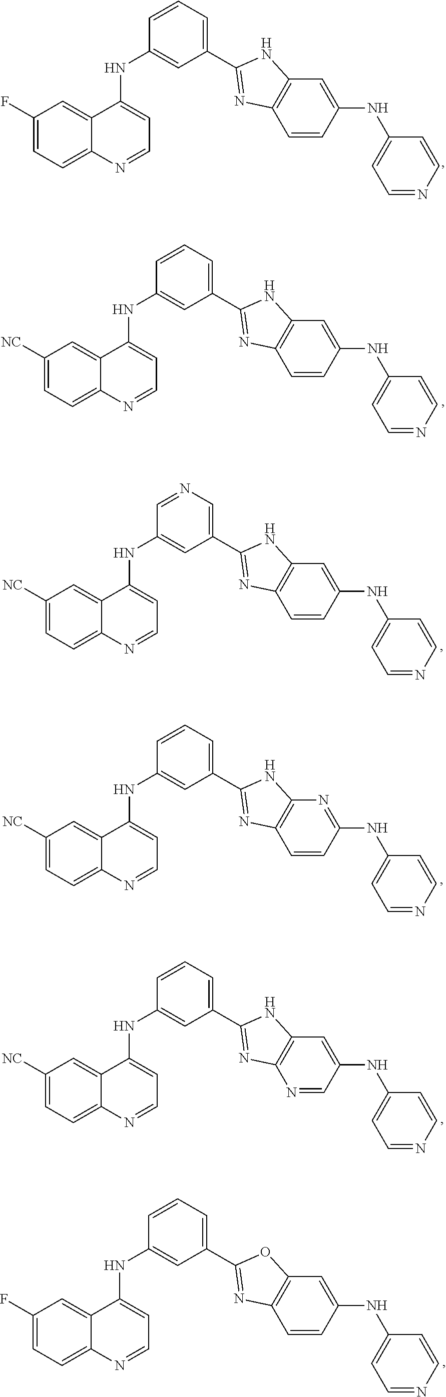 [SMILES: Fc1ccc2nccc(Nc3cccc(-c4nc5ccc(Nc6ccncc6)cc5[nH]4)c3)c2c1 ; N#Cc1ccc2nccc(Nc3cccc(-c4nc5ccc(Nc6ccncc6)cc5[nH]4)c3)c2c1 ; N#Cc1ccc2nccc(Nc3cncc(-c4nc5ccc(Nc6ccncc6)cc5[nH]4)c3)c2c1 ; N#Cc1ccc2nccc(Nc3cccc(-c4nc5ccc(Nc6ccncc6)nc5[nH]4)c3)c2c1 ; N#Cc1ccc2nccc(Nc3cccc(-c4nc5ncc(Nc6ccncc6)cc5[nH]4)c3)c2c1 ; Fc1ccc2nccc(Nc3cccc(-c4nc5ccc(Nc6ccncc6)cc5o4)c3)c2c1]

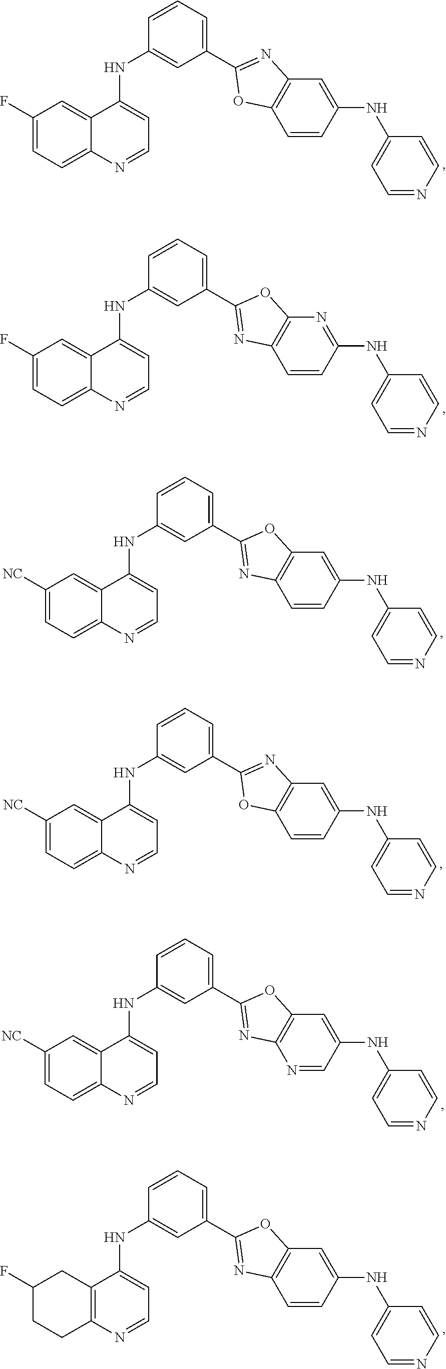 [SMILES: Fc1ccc2nccc(Nc3cccc(-c4nc5cc(Nc6ccncc6)ccc5o4)c3)c2c1 ; Fc1ccc2nccc(Nc3cccc(-c4nc5ccc(Nc6ccncc6)nc5o4)c3)c2c1 ; N#Cc1ccc2nccc(Nc3cccc(-c4nc5ccc(Nc6ccncc6)cc5o4)c3)c2c1 ; N#Cc1ccc2nccc(Nc3cccc(-c4nc5cc(Nc6ccncc6)ccc5o4)c3)c2c1 ; N#Cc1ccc2nccc(Nc3cccc(-c4nc5ncc(Nc6ccncc6)cc5o4)c3)c2c1 ; FC1CCc2nccc(Nc3cccc(-c4nc5ccc(Nc6ccncc6)cc5o4)c3)c2C1]

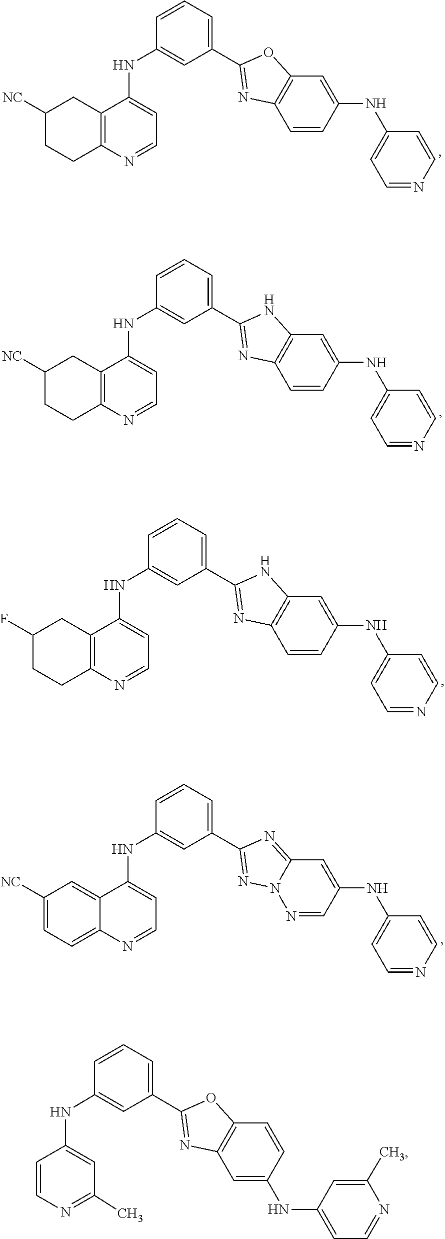 [SMILES: N#CC1CCc2nccc(Nc3cccc(-c4nc5ccc(Nc6ccncc6)cc5o4)c3)c2C1 ; N#CC1CCc2nccc(Nc3cccc(-c4nc5ccc(Nc6ccncc6)cc5[nH]4)c3)c2C1 ; FC1CCc2nccc(Nc3cccc(-c4nc5ccc(Nc6ccncc6)cc5[nH]4)c3)c2C1 ; N#Cc1ccc2nccc(Nc3cccc(-c4nc5cc(Nc6ccncc6)cnn5n4)c3)c2c1 ; Cc1cc(Nc2cccc(-c3nc4cc(Nc5ccnc(C)c5)ccc4o3)c2)ccn1]

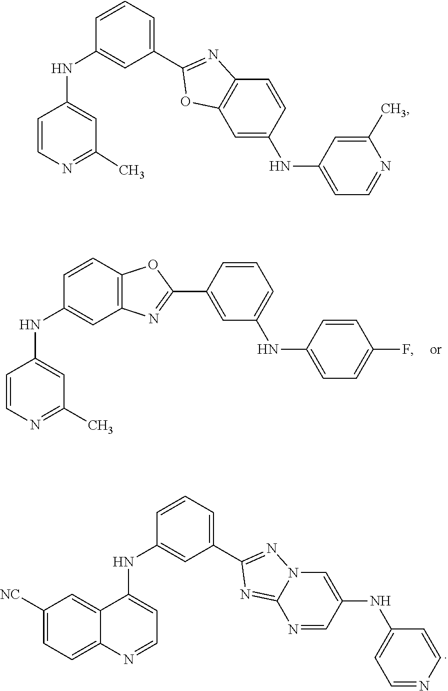 [SMILES: Cc1cc(Nc2cccc(-c3nc4ccc(Nc5ccnc(C)c5)cc4o3)c2)ccn1 ; Cc1cc(Nc2ccc3oc(-c4cccc(Nc5ccc(F)cc5)c4)nc3c2)ccn1 ; N#Cc1ccc2nccc(Nc3cccc(-c4nc5ncc(Nc6ccncc6)cn5n4)c3)c2c1]

101. The compound of claim 1, wherein the compound of Formula Ic is

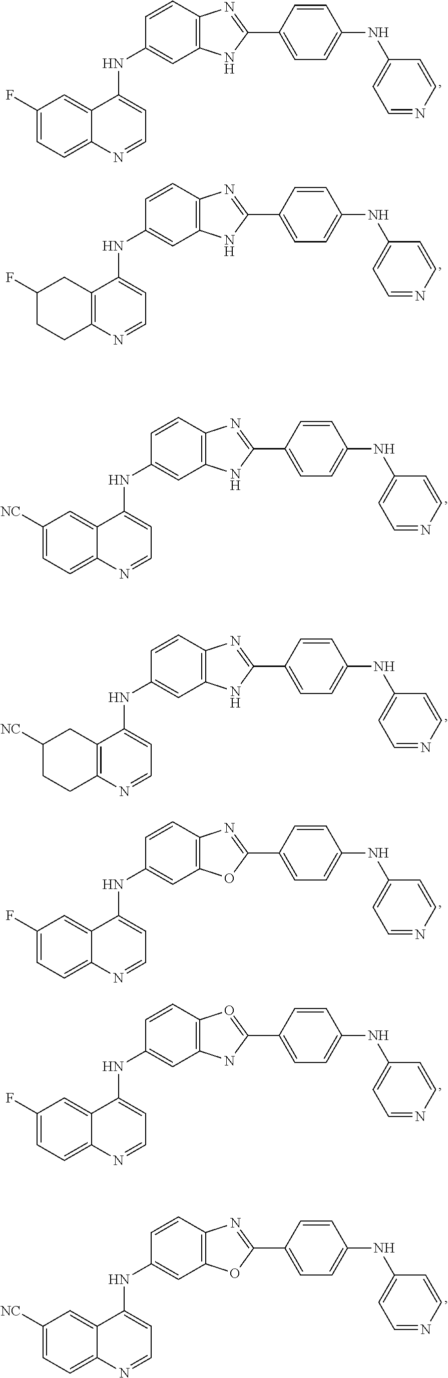

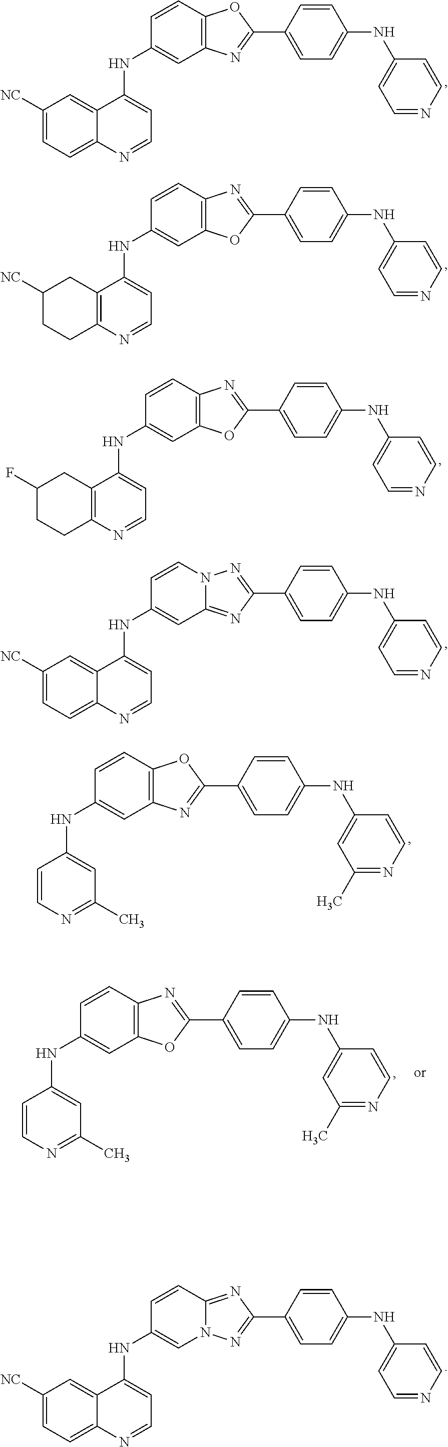

102. The compound of claim 1, wherein the compound is

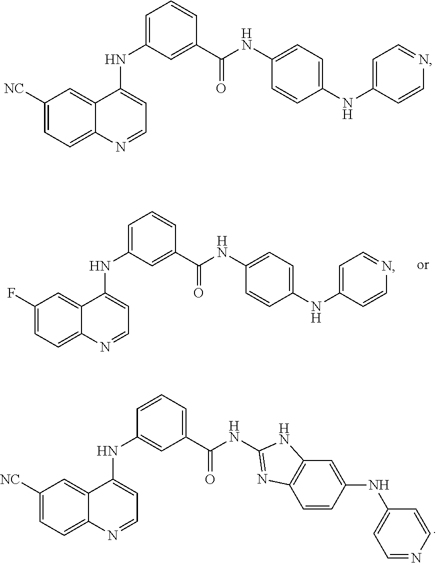 [SMILES: N#Cc1ccc2nccc(Nc3cccc(C(=O)Nc4ccc(Nc5ccncc5)cc4)c3)c2c1 ; O=C(Nc1ccc(Nc2ccncc2)cc1)c1cccc(Nc2ccnc3ccc(F)cc23)c1 ; "or" ; N#Cc1ccc2nccc(Nc3cccc(C(=O)Nc4nc5ccc(Nc6ccncc6)cc5[nH]4)c3)c2c1]

103. The compound of claim 1, wherein the compound is

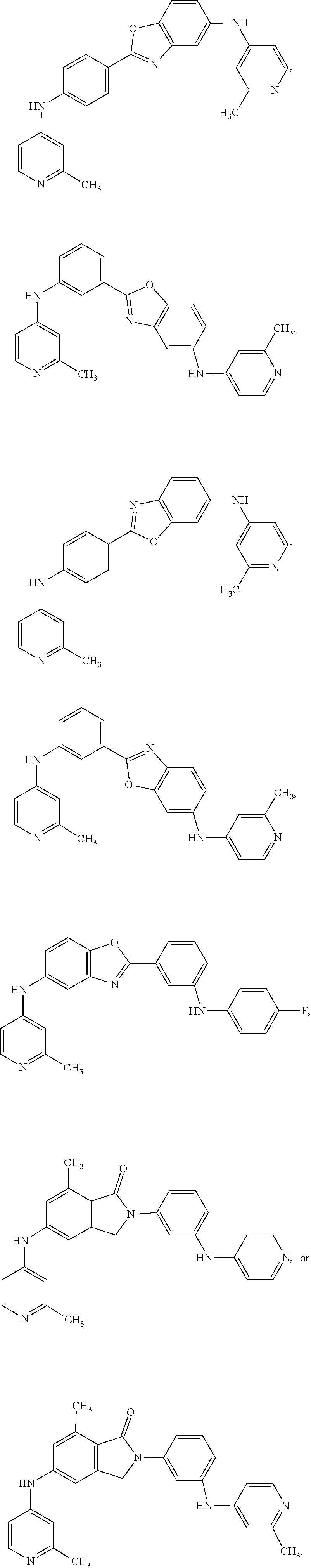

104. The compound of claim 1, wherein the compound is selected from the group consisting of compounds 2-45 in Examples 2-45, respectively.

105-136. (canceled)


SEQUENCE LISTING (3 sequences)

SEQ ID NO: 1 - DNA, 1708 bp, Homo sapiens
  source 1..1708: mol_type = unassigned DNA
aggggagtcatcatgagcgatgttaccattgtgaaggaaggttgggttcagaagaggggagaatatataaaaaactggaggccaagatacttccttttgaagacagatggctcattcataggatataaagagaaacctcaagatgtggatttaccttatcccctcaacaacttttcagtggcaaaatgccagttaatgaaaacagaacgaccaaagccaaacacatttataatcagatgtctccagtggactactgttatagagagaacatttcatgtagatactccagaggaaagggaagaatggacagaagctatccaggctgtagcagacagactgcagaggcaagaagaggagagaatgaattgtagtccaacttcacaaattgataatataggagaggaagagatggatgcctctacaacccatcataaaagaaagacaatgaatgattttgactatttgaaactactaggtaaaggcacttttgggaaagttattttggttcgagagaaggcaagtggaaaatactatgctatgaagattctgaagaaagaagtcattattgcaaaggatgaagtggcacacactctaactgaaagcagagtattaaagaacactagacatccctttttaacatccttgaaatattccttccagacaaaagaccgtttgtgttttgtgatggaatatgttaatgggggcgagctgtttttccatttgtcgagagagcgggtgttctctgaggaccgcacacgtttctatggtgcagaaattgtctctgccttggactatctacattccggaaagattgtgtaccgtgatctcaagttggagaatctaatgctggacaaagatggccacataaaaattacagattttggactttgcaaagaagggatcacagatgcagccaccatgaagacattctgtggcactccagaatatctggcaccagaggtgttagaagataatgactatggccgagcagtagactggtggggcctaggggttgtcatgtatgaaatgatgtgtgggaggttacctttctacaaccaggaccatgagaaactttttgaattaatattaatggaagacattaaatttcctcgaacactctcttcagatgcaaaatcattgctttcagggctcttgataaaggatccaaataaacgccttggtggaggaccagatgatgcaaaagaaattatgagacacagtttcttctctggagtaaactggcaagatgtatatgataaaaagcttgtacctccttttaaacctcaagtaacatctgagacagatactagatattttgatgaagaatttacagctcagactattacaataacaccacctgaaaaatatgatgaggatggtatggactgcatggacaatgagaggcggccgcatttccctcaattttcctactctgcaagtggacgagaataagtctctttcattctgctacttcactgtcatcttcaatttattactgaaaatgattcctggacatcaccagtcctagctcttacacatagcaggggcaccttccgacatcccagaccagccaagggtcctcacccctcgccacctttcaccctcatgaaaacacacatacacgcaaatacactccagtttttgtttttgcatgaaattgtatctcagtctaaggtctcatgctgttgctgctactgtcttactatta

SEQ ID NO: 2 - protein, 479 aa, Homo sapiens
MSDVTIVKEGWVQKRGEYIKNWRPRYFLLKTDGSFIGYKEKPQDVDLPYPLNNFSVAKCQLMKTERPKPNTFIIRCLQWTTVIERTFHVDTPEEREEWTEAIQAVADRLQRQEEERMNCSPTSQIDNIGEEEMDASTTHHKRKTMNDFDYLKLLGKGTFGKVILVREKASGKYYAMKILKKEVIIAKDEVAHTLTESRVLKNTRHPFLTSLKYSFQTKDRLCFVMEYVNGGELFFHLSRERVFSEDRTRFYGAEIVSALDYLHSGKIVYRDLKLENLMLDKDGHIKITDFGLCKEGITDAATMKTFCGTPEYLAPEVLEDNDYGRAVDWWGLGVVMYEMMCGRLPFYNQDHEKLFELILMEDIKFPRTLSSDAKSLLSGLLIKDPNKRLGGGPDDAKEIMRHSFFSGVNWQDVYDKKLVPPFKPQVTSETDTRYFDEEFTAQTITITPPEKYDEDGMDCMDNERRPHFPQFSYSASGRE

SEQ ID NO: 3 - protein, 478 aa, Homo sapiens
SDVTIVKEGWVQKRGEYIKNWRPRYFLLKTDGSFIGYKEKPQDVDLPYPLNNFSVAKCQLMKTERPKPNTFIIRCLQWTTVIERTFHVDTPEEREEWTEAIQAVADRLQRQEEERMNCSPTSQIDNIGEEEMDASTTHHKRKTMNDFDYLKLLGKGTFGKVILVREKASGKYYAMKILKKEVIIAKDEVAHTLTESRVLKNTRHPFLTSLKYSFQTKDRLCFVMEYVNGGELFFHLSRERVFSEDRTRFYGAEIVSALDYLHSGKIVYRDLKLENLMLDKDGHIKITDFGLCKEGITDAATMKTFCGTPEYLAPEVLEDNDYGRAVDWWGLGVVMYEMMCGRLPFYNQDHEKLFELILMEDIKFPRTLSSDAKSLLSGLLIKDPNKRLGGGPDDAKEIMRHSFFSGVNWQDVYDKKLVPPFKPQVTSETDTRYFDEEFTAQTITITPPEKYDEDGMDCMDNERRPHFPQFSYSASGRE